(19) United States
(12) Patent Application Publication
(10) Pub. No.: US 2019/0203212 A1
(43) Pub. Date: Jul. 4, 2019

(54) INDUCIBLE DNA BINDING PROTEINS AND GENOME PERTURBATION TOOLS AND APPLICATIONS THEREOF

(71) Applicants: Massachusetts Institute of Technology, Cambridge, MA (US); The Broad Institute, Inc., Cambridge, MA (US); President and Fellows of Harvard College, Cambridge, MA (US)

(72) Inventors: Feng Zhang, Cambridge, MA (US); Mark Brigham, Somerville, MA (US); Le Cong, Cambridge, MA (US); Silvana Konermann, Zurich (CH); Neville Sanjana, Cambridge, MA (US)

(73) Assignees: Massachusetts Institute of Technology, Cambridge, MA (US); The Broad Institute, Inc., Cambridge, MA (US); President and Fellows of Harvard College, Cambridge, MA (US)

(21) Appl. No.: 16/297,560

(22) Filed: Mar. 8, 2019

Related U.S. Application Data
(63) Continuation of application No. 15/388,248, filed on Dec. 22, 2016.
Application No. 15/388,248 is a continuation of application No. 14/604,641, filed on Jan. 23, 2015, now abandoned.
Application No. 14/604,641 is a continuation-in-part of application No. PCT/US13/51418, filed on Jul. 21, 2013.

(51) Int. Cl. C12N 15/63; C12N 15/62; C12N 15/86

(52) U.S. Cl. CPC C12N 15/635; C12N 15/62; C12N 2740/16043; C12N 15/86; C12N 2750/14143; C12N 15/63

(57) ABSTRACT

The present invention generally relates to methods and compositions used for the spatial and temporal control of gene expression that may use inducible transcriptional effectors. The invention particularly relates to inducible methods of altering or perturbing expression of a genomic locus of interest in a cell wherein the genomic locus may be contacted with a non-naturally occurring or engineered composition comprising a deoxyribonucleic acid (DNA) binding polypeptide.

### RELATED APPLICATIONS AND INCORPORATION BY REFERENCE

This application is a continuation of U.S. patent application Ser. No. 15/388,248 filed Dec. 22, 2016, which is a continuation of U.S. patent application Ser. No. 14/604,641 filed Jan. 23, 2015, which is a continuation-in part of international patent application Serial No. PCT/US13/51418 filed Jul. 21, 2013, which published as WO2014/018423 on Jan. 30, 2014 which claims priority to and claims benefit of US provisional patent application Serial Nos. 61/675,778 filed Jul. 25, 2012, 61/721,283 filed Nov. 1, 2012, 61/736,465 filed Dec. 12, 2012, 61/794,458 filed Mar. 15, 2013 and 61/835,973 filed Jun. 17, 2013 titled INDUCIBLE DNA BINDING PROTEINS AND GENOME PERTURBATION TOOLS AND APPLICATIONS THEREOF.

Reference is also made to U.S. Provisional Application No. 61/565,171 filed Nov. 30, 2011 and U.S. application Ser. No. 13/554,922 filed Jul. 30, 2012 and Ser. No. 13/604,945 filed Sep. 6, 2012, titled NUCLEOTIDE-SPECIFIC RECOGNITION SEQUENCES FOR DESIGNER TAL EFFECTORS.

Reference is also made to US Provisional Application Nos. 61/736,527 filed Dec. 12, 2012; 61/748,427 filed Jan. 2, 2013; 61/757,972 filed Jan. 29, 2013, 61/768,959, filed Feb. 25, 2013 and 61/791,409 filed Mar. 15, 2013, titled SYSTEMS METHODS AND COMPOSITIONS FOR SEQUENCE MANIPULATION.

Reference is also made to US Provisional Application Nos. 61/758,468 filed Jan. 30, 2013 and 61/769,046 filed Mar. 15, 2013, titled ENGINEERING AND OPTIMIZATION OF SYSTEMS, METHODS AND COMPOSITIONS FOR SEQUENCE MANIPULATION.

Reference is also made to U.S. Provisional Application Nos. 61/835,931; 61/835,936; 61/836,080; 61/836,101; 61/836,123 and 61/836,127 filed Jun. 17, 2013.

Reference is also made to U.S. Provisional Application No. 61/842,322, filed Jul. 2, 2013, titled CRISPR-CAS SYSTEMS AND METHODS FOR ALTERING EXPRESSION OF GENE PRODUCTS and U.S. Provisional Application No. 61/847,537, filed Jul. 17, 2013, titled DELIVERY, ENGINEERING AND OPTIMIZATION OF SYSTEMS, METHODS AND COMPOSITIONS FOR SEQUENCE MANIPULATION AND APPLICATIONS.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under R01NS073124 and Pioneer Award 1MH100706 awarded by the National Institutes of Health. The Government has certain rights in the invention.

### FIELD OF THE INVENTION

The present invention generally relates to methods and compositions used for the spatial and temporal control of gene expression, such as genome perturbation, that may use inducible transcriptional effectors.

### SEQUENCE LISTING

The instant application contains a Sequence Listing which has been submitted electronically in ASCII format and is hereby incorporated by reference in its entirety. Said ASCII copy, created on Feb. 16, 2015, is named 44790.04.2005_SL.txt and is 827,181 bytes in size.

### BACKGROUND OF THE INVENTION

Normal gene expression is a dynamic process with carefully orchestrated temporal and spatial components, the precision of which are necessary for normal development, homeostasis, and advancement of the organism. In turn, the dysregulation of required gene expression patterns, either by increased, decreased, or altered function of a gene or set of genes, has been linked to a wide array of pathologies. Technologies capable of modulating gene expression in a spatiotemporally precise fashion will enable the elucidation of the genetic cues responsible for normal biological processes and disease mechanisms. To address this technological need, Applicants developed inducible molecular tools that may regulate gene expression, in particular, light-inducible transcriptional effectors (LITEs), which provide light-mediated control of endogenous gene expression.

Inducible gene expression systems have typically been designed to allow for chemically induced activation of an inserted open reading frame or shRNA sequence, resulting in gene overexpression or repression, respectively. Disadvantages of using open reading frames for overexpression include loss of splice variation and limitation of gene size. Gene repression via RNA interference, despite its transformative power in human biology, can be hindered by complicated off-target effects. Certain inducible systems including estrogen, ecdysone, and FKBP12/FRAP based systems are known to activate off-target endogenous genes. The potentially deleterious effects of long-term antibiotic treatment can complicate the use of tetracycline transactivator (TET) based systems. In vivo, the temporal precision of these chemically inducible systems is dependent upon the kinetics of inducing agent uptake and elimination. Further, because inducing agents are generally delivered systemically, the spatial precision of such systems is bounded by the precision of exogenous vector delivery.

US Patent Publication No. 20030049799 relates to engineered stimulus-responsive switches to cause a detectable output in response to a preselected stimulus.

There is an evident need for methods and compositions that allow for efficient and precise spatial and temporal control of a genomic locus of interest. These methods and compositions may provide for the regulation and modulation of genomic expression both in vivo and in vitro as well as provide for novel treatment methods for a number of disease pathologies.

Citation or identification of any document in this application is not an admission that such document is available as prior art to the present invention.

### SUMMARY OF THE INVENTION

In one aspect the invention provides a non-naturally occurring or engineered TALE or CRISPR-Cas system which may comprise at least one switch wherein the activity of said TALE or CRISPR-Cas system is controlled by contact with at least one inducer energy source as to the switch. In an embodiment of the invention the control as to the at least one switch or the activity of said TALE or CRISPR-Cas system may be activated, enhanced, terminated or repressed. The contact with the at least one inducer energy source may result in a first effect and a second effect. The first effect may be one or more of nuclear import, nuclear export, recruitment of a secondary component (such as an effector molecule), conformational change (of protein, DNA or RNA), cleavage, release of cargo (such as a caged molecule or a co-factor), association or dissociation. The second effect may be one or more of activation, enhancement, termination or repression of the control as to the at least one switch or the activity of said TALE or CRISPR-Cas system. In one embodiment the first effect and the second effect may occur in a cascade.

In another aspect of the invention the TALE or CRISPR-Cas system may further comprise at least one nuclear localization signal (NLS), nuclear export signal (NES), functional domain, flexible linker, mutation, deletion, alteration or truncation. The one or more of the NLS, the NES or the functional domain may be conditionally activated or inactivated. In another embodiment, the mutation may be one or more of a mutation in a transcription factor homology region, a mutation in a DNA binding domain (such as mutating basic residues of a basic helix loop helix), a mutation in an endogenous NLS or a mutation in an endogenous NES. The invention comprehends that the inducer energy source may be heat, ultrasound, electromagnetic energy or chemical. In a preferred embodiment of the invention, the inducer energy source may be an antibiotic, a small molecule, a hormone, a hormone derivative, a steroid or a steroid derivative. In a more preferred embodiment, the inducer energy source maybe abscisic acid (ABA), doxycycline (DOX), cumate, rapamycin, 4-hydroxytamoxifen (4OHT), estrogen or ecdysone. The invention provides that the at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems.

In one aspect of the invention the inducer energy source is electromagnetic energy. The electromagnetic energy may be a component of visible light having a wavelength in the range of 450 nm-700 nm. In a preferred embodiment the component of visible light may have a wavelength in the range of 450 nm-500 nm and may be blue light. The blue light may have an intensity of at least 0.2 mW/cm², or more preferably at least 4 mW/cm². In another embodiment, the component of visible light may have a wavelength in the range of 620-700 nm and is red light.

The invention comprehends systems wherein the at least one functional domain may be selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease.

The invention also provides for use of the system for perturbing a genomic or epigenomic locus of interest. Also provided are uses of the system for the preparation of a pharmaceutical compound.

In a further aspect, the invention provides a method of controlling a non-naturally occurring or engineered TALE or CRISPR-Cas system, comprising providing said TALE or CRISPR-Cas system comprising at least one switch wherein the activity of said TALE or CRISPR-Cas system is controlled by contact with at least one inducer energy source as to the switch.

In an embodiment of the invention, the invention provides methods wherein the control as to the at least one switch or the activity of said TALE or CRISPR-Cas system may be activated, enhanced, terminated or repressed. The contact with the at least one inducer energy source may result in a first effect and a second effect. The first effect may be one or more of nuclear import, nuclear export, recruitment of a secondary component (such as an effector molecule), conformational change (of protein, DNA or RNA), cleavage, release of cargo (such as a caged molecule or a co-factor), association or dissociation. The second effect may be one or more of activation, enhancement, termination or repression of the control as to the at least one switch or the activity of said TALE or CRISPR-Cas system. In one embodiment the first effect and the second effect may occur in a cascade.

In another aspect of the methods of the invention the TALE or CRISPR-Cas system may further comprise at least one nuclear localization signal (NLS), nuclear export signal (NES), functional domain, flexible linker, mutation, deletion, alteration or truncation. The one or more of the NLS, the NES or the functional domain may be conditionally activated or inactivated. In another embodiment, the mutation may be one or more of a mutation in a transcription factor homology region, a mutation in a DNA binding domain (such as mutating basic residues of a basic helix loop helix), a mutation in an endogenous NLS or a mutation in an endogenous NES. The invention comprehends that the inducer energy source may be heat, ultrasound, electromagnetic energy or chemical. In a preferred embodiment of the invention, the inducer energy source may be an antibiotic, a small molecule, a hormone, a hormone derivative, a steroid or a steroid derivative. In a more preferred embodiment, the inducer energy source maybe abscisic acid (ABA), doxycycline (DOX), cumate, rapamycin, 4-hydroxytamoxifen (4OHT), estrogen or ecdysone. The invention provides that the at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems.

In one aspect of the methods of the invention the inducer energy source is electromagnetic energy. The electromagnetic energy may be a component of visible light having a wavelength in the range of 450 nm-700 nm. In a preferred embodiment the component of visible light may have a wavelength in the range of 450 nm-500 nm and may be blue light. The blue light may have an intensity of at least 0.2 mW/cm², or more preferably at least 4 mW/cm². In another embodiment, the component of visible light may have a wavelength in the range of 620-700 nm and is red light.

The invention comprehends methods wherein the at least one functional domain may be selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease.

Further aspects of the invention provides for systems or methods as described herein wherein the TALE system comprises a DNA binding polypeptide comprising:

(i) a DNA binding domain comprising at least five or more Transcription activator-like effector (TALE) monomers and at least one or more half-monomers specifically ordered to target a locus of interest or
at least one or more effector domains
linked to an energy sensitive protein or fragment thereof, wherein the energy sensitive protein or fragment thereof undergoes a conformational change upon induction by an inducer energy source allowing it to bind an interacting partner, and/or
(ii) a DNA binding domain comprising at least one or more TALE monomers or half-monomers specifically ordered to target the locus of interest or
at least one or more effector domains
linked to the interacting partner, wherein the energy sensitive protein or fragment thereof binds to the interacting partner upon induction by the inducer energy source.

The systems and methods of the invention provide for the DNA binding polypeptide comprising a (a) a N-terminal capping region (b) a DNA binding domain comprising at least 5 to 40 Transcription activator-like effector (TALE) monomers and at least one or more half-monomers specifically ordered to target the locus of interest, and (c) a C-terminal capping region wherein (a), (b) and (c) may be arranged in a predetermined N-terminus to C-terminus orientation, wherein the genomic locus comprises a target DNA sequence 5′-T₀N₁N₂ . . . N_{z} N_{z+1}-3′, where T₀ and N=A, G, T or C, wherein the target DNA sequence binds to the DNA binding domain, and the DNA binding domain may comprise (X₁₁₋₁-X₁₂X₁₃-X_{14-33 or 34 or 35})z, wherein X₁₋₁₁ is a chain of 11 contiguous amino acids, wherein X₁₂X₁₃ is a repeat variable diresidue (RVD), wherein X_{14-33 or 34 or 35} is a chain of 21, 22 or 23 contiguous amino acids, wherein z may be at least 5 to 40, wherein the polypeptide may be encoded by and translated from a codon optimized nucleic acid molecule so that the polypeptide preferentially binds to DNA of the locus of interest.

In a further embodiment, the system or method of the invention provides the N-terminal capping region or fragment thereof comprises 147 contiguous amino acids of a wild type N-terminal capping region, or the C-terminal capping region or fragment thereof comprises 68 contiguous amino acids of a wild type C-terminal capping region, or the N-terminal capping region or fragment thereof comprises 136 contiguous amino acids of a wild type N-terminal capping region and the C-terminal capping region or fragment thereof comprises 183 contiguous amino acids of a wild type C-terminal capping region. In another embodiment, the at least one RVD may be selected from the group consisting of (a) HH, KH, NH, NK, NQ, RH, RN, SS, NN, SN, KN for recognition of guanine (G); (b) NI, KI, RI, HI, SI for recognition of adenine (A); (c) NG, HG, KG, RG for recognition of thymine (T); (d) RD, SD, HD, ND, KD, YG for recognition of cytosine (C); (e) NV, HN for recognition of A or G; and (f) H*, HA, KA, N*, NA, NC, NS, RA, S*for recognition of A or T or G or C, wherein (*) means that the amino acid at X13 is absent.

In yet another embodiment the at least one RVD may be selected from the group consisting of (a) HH, KH, NH, NK, NQ, RH, RN, SS for recognition of guanine (G); (b) SI for recognition of adenine (A); (c) HG, KG, RG for recognition of thymine (T); (d) RD, SD for recognition of cytosine (C); (e) NV, HN for recognition of A or G and (f) H*, HA, KA, N*, NA, NC, NS, RA, S*for recognition of A or T or G or C, wherein (*) means that the amino acid at X13 is absent. In a preferred embodiment, the RVD for the recognition of G is RN, NH, RH or KH; or the RVD for the recognition of A is SI; or the RVD for the recognition of T is KG or RG; and the RVD for the recognition of C is SD or RD. In yet another embodiment, at least one of the following is present [LTLD] (SEQ ID NO: 1) or [LTLA] (SEQ ID NO: 2) or [LTQV] (SEQ ID NO: 3) at X1-4, or [EQHG] (SEQ ID NO: 4) or [RDHG] (SEQ ID NO: 5) at positions X30-33 or X31-34 or X32-35.

In an aspect of the invention the TALE system is packaged into a AAV or a lentivirus vector.

Further aspects of the invention provides for systems or methods as described herein wherein the CRISPR system may comprise a vector system comprising: a) a first regulatory element operably linked to a CRISPR-Cas system guide RNA that targets a locus of interest, b) a second regulatory inducible element operably linked to a Cas protein, wherein components (a) and (b) may be located on same or different vectors of the system, wherein the guide RNA targets DNA of the locus of interest, wherein the Cas protein and the guide RNA do not naturally occur together. In a preferred embodiment of the invention, the Cas protein is a Cas9 enzyme. The invention also provides for the vector being a AAV or a lentivirus.

The invention particularly relates to inducible methods of altering expression of a genomic locus of interest and to compositions that inducibly alter expression of a genomic locus of interest wherein the genomic locus may be contacted with a non-naturally occurring or engineered composition comprising a deoxyribonucleic acid (DNA) binding polypeptide.

This polypeptide may include a DNA binding domain comprising at least five or more Transcription activator-like effector (TALE) monomers and at least one or more half-monomers specifically ordered to target the genomic locus of interest or at least one or more effector domains linked to an energy sensitive protein or fragment thereof. The energy sensitive protein or fragment thereof may undergo a conformational change upon induction by an energy source allowing it to bind an interacting partner. The polypeptide may also include a DNA binding domain comprising at least one or more variant TALE monomers or half-monomers specifically ordered to target the genomic locus of interest or at least one or more effector domains linked to the interacting partner, wherein the energy sensitive protein or fragment thereof may bind to the interacting partner upon induction by the energy source. The method may also include applying the energy source and determining that the expression of the genomic locus is altered. In preferred embodiments of the invention the genomic locus may be in a cell.

The invention also relates to inducible methods of repressing expression of a genomic locus of interest and to compositions that inducibly repress expression of a genomic locus of interest wherein the genomic locus may be contacted with a non-naturally occurring or engineered composition comprising a DNA binding polypeptide.

The polypeptide may include a DNA binding domain comprising at least five or more Transcription activator-like effector (TALE) monomers and at least one or more half-monomers specifically ordered to target the genomic locus of interest or at least one or more repressor domains linked to an energy sensitive protein or fragment thereof. The energy sensitive protein or fragment thereof may undergo a conformational change upon induction by an energy source allowing it to bind an interacting partner. The polypeptide may also include a DNA binding domain comprising at least one or more variant TALE monomers or half-monomers specifically ordered to target the genomic locus of interest or at least one or more effector domains linked to the interacting partner, wherein the energy sensitive protein or fragment thereof may bind to the interacting partner upon induction by the energy source. The method may also include applying the energy source and determining that the expression of the genomic locus is repressed. In preferred embodiments of the invention the genomic locus may be in a cell.

The invention also relates to inducible methods of activating expression of a genomic locus of interest and to compositions that inducibly activate expression of a genomic locus of interest wherein the genomic locus may be contacted with a non-naturally occurring or engineered composition comprising a DNA binding polypeptide.

The polypeptide may include a DNA binding domain comprising at least five or more TALE monomers and at least one or more half-monomers specifically ordered to target the genomic locus of interest or at least one or more activator domains linked to an energy sensitive protein or fragment thereof. The energy sensitive protein or fragment thereof may undergo a conformational change upon induction by an energy source allowing it to bind an interacting partner. The polypeptide may also include a DNA binding domain comprising at least one or more variant TALE monomers or half-monomers specifically ordered to target the genomic locus of interest or at least one or more effector domains linked to the interacting partner, wherein the energy sensitive protein or fragment thereof may bind to the interacting partner upon induction by the energy source. The method may also include applying the energy source and determining that the expression of the genomic locus is activated. In preferred embodiments of the invention the genomic locus may be in a cell.

In another preferred embodiment of the invention, the inducible effector may be a Light Inducible Transcriptional Effector (LITE). The modularity of the LITE system allows for any number of effector domains to be employed for transcriptional modulation.

In yet another preferred embodiment of the invention, the inducible effector may be a chemical.

The present invention also contemplates an inducible multiplex genome engineering using CRISPR (clustered regularly interspaced short palindromic repeats)/Cas systems.

The present invention also encompasses nucleic acid encoding the polypeptides of the present invention. The nucleic acid may comprise a promoter, advantageously human Synapsin I promoter (hSyn). In a particularly advantageous embodiment, the nucleic acid may be packaged into an adeno associated viral vector (AAV).

The invention further also relates to methods of treatment or therapy that encompass the methods and compositions described herein.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows a schematic indicating the need for spatial and temporal precision.

FIG. 2 shows transcription activator like effectors (TALEs). TALEs consist of 34 aa repeats at the core of their sequence. Each repeat corresponds to a base in the target DNA that is bound by the TALE. Repeats differ only by 2 variable amino acids at positions 12 and 13. The code of this correspondence has been elucidated (Boch, J et al., Science, 2009 and Moscou, M et al., Science, 2009) and is shown in this figure. Applicants have developed a method for the synthesis of designer TALEs incorporating this code and capable of binding a sequence of choice within the genome (Zhang, F et al., Nature Biotechnology, 2011). FIG. 2 discloses SEQ ID NOS 212-213, respectively, in order of appearance.

FIG. 3 shows a design of a LITE: TALE/Cryptochrome transcriptional activation. Each LITE is a two-component system which may comprise a TALE fused to CRY2 and the cryptochrome binding partner CIB1 fused to VP64, a transcription activor. In the inactive state, the TALE localizes its fused CRY2 domain to the promoter region of the gene of interest. At this point, CIB1 is unable to bind CRY2, leaving the CIB1-VP64 unbound in the nuclear space. Upon stimulation with 488 nm (blue) light, CRY2 undergoes a conformational change, revealing its CIB1 binding site (Liu, H et al., Science, 2008). Rapid binding of CIB1 results in recruitment of the fused VP64 domain, which induces transcription of the target gene.

FIG. 4 shows effects of cryptochrome dimer truncations on LITE activity. Truncations known to alter the activity of CRY2 and CIB1 (Kennedy M et al., Nature Methods 2010) were compared against the full length proteins. A LITE targeted to the promoter of Neurog2 was tested in Neuro-2a cells for each combination of domains. Following stimulation with 488 nm light, transcript levels of Neurog2 were quantified using qPCR for stimulated and unstimulated samples.

FIG. 5 shows a light-intensity dependent response of KLF4 LITE.

FIG. 6 shows activation kinetics of Neurog2 LITE and inactivation kinetics of Neurog2 LITE.

FIG. 7A shows the base-preference of various RVDs as determined using the Applicants' RVD screening system.

FIG. 7B shows the base-preference of additional RVDs as determined using the Applicants' RVD screening system.

FIGS. 8A-D show in (a) Natural structure of TALEs derived from Xanthomonas sp. Each DNA-binding module consists of 34 amino acids, where the RVDs in the 12th and 13th amino acid positions of each repeat specify the DNA base being targeted according to the cipher NG=T, HD=C, NI=A, and NN=G or A. The DNA-binding modules are flanked by nonrepetitive N and C termini, which carry the translocation, nuclear localization (NLS) and transcription activation (AD) domains. A cryptic signal within the N terminus specifies a thymine as the first base of the target site. (b) The TALE toolbox allows rapid and inexpensive construction of custom TALE-TFs and TALENs. The kit consists of 12 plasmids in total: four monomer plasmids to be used as templates for PCR amplification, four TALE-TF and four TALEN cloning backbones corresponding to four different bases targeted by the 0.5 repeat. CMV, cytomegalovirus promoter; N term, nonrepetitive N terminus from the Hax3 TALE; C term, nonrepetitive C terminus from the Hax3 TALE; BsaI, type IIs restriction sites used for the insertion of custom TALE DNA-binding domains; ccdB+CmR, negative selection cassette containing the ccdB negative selection gene and chloramphenicol resistance gene; NLS, nuclear localization signal; VP64, synthetic transcriptional activator derived from VP16 protein of herpes simplex virus; 2A, 2A self-cleavage linker; EGFP, enhanced green fluorescent protein; polyA signal, polyadenylation signal; FokI, catalytic domain from the FokI endonuclease. (c) TALEs may be used to generate custom TALE-TFs and modulate the transcription of endogenous genes from the genome. The TALE DNA-binding domain is fused to the synthetic VP64 transcriptional activator, which recruits RNA polymerase and other factors needed to initiate transcription. (d) TALENs may be used to generate site-specific double-strand breaks to facilitate genome editing through nonhomologous repair or homology directed repair. Two TALENs target a pair of binding sites flanking a 16-bp spacer. The left and right TALENs recognize the top and bottom strands of the target sites, respectively. Each TALE DNA-binding domain is fused to the catalytic domain of FokI endonuclease; when FokI dimerizes, it cuts the DNA in the region between the left and right TALEN-binding sites. FIG. 8A discloses SEQ ID NOS 212-213, respectively, in order of appearance.

FIG. 9A-F shows a table listing monomer sequences (SEQ ID NOS 214-444, respectively, in order of appearance) (excluding the RVDs at positions 12 and 13) and the frequency with which monomers having a particular sequence occur.

FIG. 10 shows the comparison of the effect of non-RVD amino acid on TALE activity. FIG. 10 discloses SEQ ID NOS 215, 214, 221, 218, 244, 445, 214, 219, 334, 446, 251, and 447, respectively, in order of appearance.

FIG. 11 shows an activator screen comparing levels of activation between VP64, p65 and VP16.

FIGS. 12A-D show the development of a TALE transcriptional repressor architecture. (a) Design of SOX2 TALE for TALE repressor screening. A TALE targeting a 14 bp sequence within the SOX2 locus of the human genome was synthesized. (b) List of all repressors screened and their host origin (left). Eight different candidate repressor domains were fused to the C-term of the SOX2 TALE. (c) The fold decrease of endogenous SOX2 mRNA is measured using qRTPCR by dividing the SOX2 mRNA levels in mock transfected cells by SOX2 mRNA levels in cells transfected with each candidate TALE repressor. (d) Transcriptional repression of endogenous CACNA1C. TALEs using NN, NK, and NH as the G-targeting RVD were constructed to target a 18 bp target site within the human CACNA1C locus. Each TALE is fused to the SID repression domain. NLS, nuclear localization signal; KRAB, Krüppel-associated box; SID, mSin interaction domain. All results are collected from three independent experiments in HEK 293FT cells. Error bars indicate s.e.m.; n=3. * p<0.05, Student's t test. FIGS. 12A and 12D disclose SEQ ID NOS 448 and 449, respectively.

FIGS. 13A-C shows the optimization of TALE transcriptional repressor architecture using SID and SID4X. (a) Design of p11 TALE for testing of TALE repressor architecture. A TALE targeting a 20 bp sequence (p11 TALE binding site) within the p11 (s100a10) locus of the mouse (Mus musculus) genome was synthesized. (b) Transcriptional repression of endogenous mouse p11 mRNA. TALEs targeting the mouse p11 locus harboring two different truncations of the wild type TALE architecture were fused to different repressor domains as indicated on the x-axis. The value in the bracket indicate the number of amino acids at the N- and C-termini of the TALE DNA binding domain flanking the DNA binding repeats, followed by the repressor domain used in the construct. The endogenous p11 mRNA levels were measured using qRT-PCR and normalized to the level in the negative control cells transfected with a GFP-encoding construct. (c) Fold of transcriptional repression of endogenous mouse p11. The fold decrease of endogenous p1 mRNA is measured using qRT-PCR through dividing the p11 mRNA levels in cells transfected with a negative control GFP construct by p11 mRNA levels in cells transfected with each candidate TALE repressors. The labeling of the constructs along the x-axis is the same as previous panel. NLS, nuclear localization signal; SID, mSin interaction domain; SID4X, an optimized four-time tandem repeats of SID domain linked by short peptide linkers. All results are collected from three independent experiments in Neuro2A cells. Error bars indicate s.e.m.; n=3. *** p<0.001, Student's t test. FIG. 13A discloses SEQ ID NO: 450.

FIG. 14A-D shows a comparison of two different types of TALE architecture.

FIGS. 15A-C show a chemically inducible TALE ABA inducible system. ABI (ABA insensitive 1) and PYL (PYL protein: pyrabactin resistance (PYR)/PYR1-like (PYL)) are domains from two proteins listed below that will dimerize upon binding of plant hormone Abscisic Acid (ABA). This plant hormone is a small molecule chemical that Applicants used in Applicants' inducible TALE system. In this system, the TALE DNA-binding polypeptide is fused to the ABI domain, whereas the VP64 activation domain or SID repressor domain or any effector domains are linked to the PYL domain. Thus, upon the induction by the presence of ABA molecule, the two interacting domains, ABI and PYL, will dimerize and allow the TALE to be linked to the effector domains to perform its activity in regulating target gene expression.

FIGS. 16A-B show a chemically inducible TALE 4OHT inducible system.

FIG. 17 depicts an effect of cryptochrome2 heterodimer orientation on LITE functionality.

FIG. 18 depicts mGlur2 LITE activity in mouse cortical neuron culture.

FIG. 19 depicts transduction of primary mouse neurons with LITE AAV vectors.

FIG. 20 depicts expression of LITE component in vivo.

FIG. 21 depicts an improved design of the construct where the specific NES peptide sequence used is LDLASLIL (SEQ ID NO: 6).

FIG. 22 depicts Sox2 mRNA levels in the absence and presence of 40H tamoxifen.

FIGS. 23A-E depict a Type II CRISPR locus from Streptococcus pyogenes SF370 can be reconstituted in mammalian cells to facilitate targeted DSBs of DNA. (A) Engineering of SpCas9 and SpRNase III with NLSs enables import into the mammalian nucleus. (B) Mammalian expression of SpCas9 and SpRNase III are driven by the EF1a promoter, whereas tracrRNA and pre-crRNA array (DR-Spacer-DR) are driven by the U6 promoter. A protospacer (blue highlight) from the human EMX1 locus with PAM is used as template for the spacer in the pre-crRNA array. (C) Schematic representation of base pairing between target locus and EMX1-targeting crRNA. Red arrow indicates putative cleavage site. (D) SURVEYOR assay for SpCas9-mediated indels. (E) An example chromatogram showing a micro-deletion, as well as representative sequences of mutated alleles identified from 187 clonal amplicons. Red dashes, deleted bases; red bases, insertions or mutations. Scale bar=10 μm. FIG. 23B discloses SEQ ID NO: 451, FIG. 23C discloses SEQ ID NOS 452-453, and FIG. 23E discloses SEQ ID NOS 454-461, all respectively, in order of appearance.

FIGS. 24A-C depict a SpCas9 can be reprogrammed to target multiple genomic loci in mammalian cells. (A) Schematic of the human EMX1 locus showing the location of five protospacers, indicated by blue lines with corresponding PAM in magenta. (B) Schematic of the pre-crRNA:tracrRNA complex (top) showing hybridization between the direct repeat (gray) region of the pre-crRNA and tracrRNA. Schematic of a chimeric RNA design (M. Jinek et al., A programmable dual-RNA-guided DNA endonuclease in adaptive bacterial immunity. Science 337, 816 (Aug. 17, 2012)) (bottom). tracrRNA sequence is shown in red and the 20 bp spacer sequence in blue. (C) SURVEYOR assay comparing the efficacy of Cas9-mediated cleavage at five protospacers in the human EMX1 locus. Each protospacer is targeted using either processed pre-crRNA:tracrRNA complex (crRNA) or chimeric RNA (chiRNA). FIG. 24A discloses SEQ ID NO: 462 and FIG. 24B discloses SEQ ID NOS 463-465, respectively, in order of appearance.

FIGS. 25A-D depict an evaluation of the SpCas9 specificity and comparison of efficiency with TALENs. (A) EMX1-targeting chimeric crRNAs with single point mutations were generated to evaluate the effects of spacer-protospacer mismatches. (B) SURVEYOR assay comparing the cleavage efficiency of different mutant chimeric RNAs. (C) Schematic showing the design of TALENs targeting EMX1. (D) SURVEYOR gel comparing the efficiency of TALEN and SpCas9 (N=3). FIG. 25A discloses SEQ ID NOS 466-478, respectively, in order of appearance, and FIG. 25C discloses SEQ ID NO: 466.

FIGS. 26A-G depict applications of Cas9 for homologous recombination and multiplex genome engineering. (A) Mutation of the RuvC I domain converts Cas9 into a nicking enzyme (SpCas9n) (B) Co-expression of EMX1-targeting chimeric RNA with SpCas9 leads to indels, whereas SpCas9n does not (N=3). (C) Schematic representation of the recombination strategy. A repair template is designed to insert restriction sites into EMX1 locus. Primers used to amplify the modified region are shown as red arrows. (D) Restriction fragments length polymorphism gel analysis. Arrows indicate fragments generated by HindIII digestion. (E) Example chromatogram showing successful recombination. (F) SpCas9 can facilitate multiplex genome modification using a crRNA array containing two spacers targeting EMX1 and PVALB. Schematic showing the design of the crRNA array (top). Both spacers mediate efficient protospacer cleavage (bottom). (G) SpCas9 can be used to achieve precise genomic deletion. Two spacers targeting EMX1 (top) mediated a 118 bp genomic deletion (bottom). FIG. 26E discloses SEQ ID NO: 479, FIG. 26F discloses SEQ ID NOS 480-481, and FIG. 26G discloses SEQ ID NOS 482-486, respectively, in order of appearance.

FIG. 27 depicts a schematic of the type II CRISPR-mediated DNA double-strand break. The type II CRISPR locus from Streptococcus pyogenes SF370 contains a cluster of four genes, Cas9, Cas1, Cas2, and Csn1, as well as two non-coding RNA elements, tracrRNA and a characteristic array of repetitive sequences (direct repeats) interspaced by short stretches of non-repetitive sequences (spacers, 30 bp each) (15-18, 30, 31). Each spacer is typically derived from foreign genetic material (protospacer), and directs the specificity of CRISPR-mediated nucleic acid cleavage. In the target nucleic acid, each protospacer is associated with a protospacer adjacent motif (PAM) whose recognition is specific to individual CRISPR systems (22, 23). The Type II CRISPR system carries out targeted DNA double-strand break (DSB) in sequential steps (M. Jinek et al., Science 337, 816 (Aug. 17, 2012); Gasiunas, R. et al. Proc Natl Acad Sci USA 109, E2579 (Sep. 25, 2012); J. E. Garneau et al., Nature 468, 67 (Nov. 4, 2010); R. Sapranauskas et al., Nucleic Acids Res 39, 9275 (November, 2011); A. H. Magadan et al. PLoS One 7, e40913 (2012)). First, the pre-crRNA array and tracrRNA are transcribed from the CRISPR locus. Second, tracrRNA hybridizes to the direct repeats of pre-crRNA and associates with Cas9 as a duplex, which mediates the processing of the pre-crRNA into mature crRNAs containing individual, truncated spacer sequences. Third, the mature crRNA:tracrRNA duplex directs Cas9 to the DNA target consisting of the protospacer and the requisite PAM via heteroduplex formation between the spacer region of the crRNA and the protospacer DNA. Finally, Cas9 mediates cleavage of target DNA upstream of PAM to create a DSB within the protospacer.

FIGS. 28A-C depict a comparison of different tracrRNA transcripts for Cas9-mediated gene targeting. (A) Schematic showing the design and sequences of two tracrRNA transcripts tested (short and long). Each transcript is driven by a U6 promoter. Transcription start site is marked as +1 and transcription terminator is as indicated. Blue line indicates the region whose reverse-complement sequence is used to generate northern blot probes for tracrRNA detection. (B) SURVEYOR assay comparing the efficiency of hSpCas9-mediated cleavage of the EMX1 locus. Two biological replicas are shown for each tracrRNA transcript. (C) Northern blot analysis of total RNA extracted from 293FT cells transfected with U6 expression constructs carrying long or short tracrRNA, as well as SpCas9 and DR-EMX1(1)-DR. Left and right panels are from 293FT cells transfected without or with SpRNase III respectively. U6 indicate loading control blotted with a probe targeting human U6 snRNA. Transfection of the short tracrRNA expression construct led to abundant levels of the processed form of tracrRNA (˜75 bp) (E. Deltcheva et al., Nature 471, 602 (Mar. 31, 2011)). Very low amounts of long tracrRNA are detected on the Northern blot. As a result of these experiments, Applicants chose to use short tracrRNA for application in mammalian cells. FIG. 28A discloses SEQ ID NOS 487-488, respectively, in order of appearance.

FIG. 29 depicts a SURVEYOR assay for detection of double strand break-induced micro insertions and deletions (D. Y. Guschin et al. Methods Mol Biol 649, 247 (2010)). Schematic of the SURVEYOR assay used to determine Cas9-mediated cleavage efficiency. First, genomic PCR (gPCR) is used to amplify the Cas9 target region from a heterogeneous population of modified and unmodified cells, and the gPCR products are reannealed slowly to generate heteroduplexes. The reannealed heteroduplexes are cleaved by SURVEYOR nuclease, whereas homoduplexes are left intact. Cas9-mediated cleavage efficiency (% indel) is calculated based on the fraction of cleaved DNA.

FIG. 30A-B depict a Northern blot analysis of crRNA processing in mammalian cells. (A) Schematic showing the expression vector for a single spacer flanked by two direct repeats (DR-EMX1(1)-DR). The 30 bp spacer targeting the human EMX1 locus protospacer 1 (Table 1) is shown in blue and direct repeats are in shown in gray. Orange line indicates the region whose reversecomplement sequence is used to generate northern blot probes for EMX1(1) crRNA detection. (B) Northern blot analysis of total RNA extracted from 293FT cells transfected with U6 expression constructs carrying DR-EMX1(1)-DR. Left and right panels are from 293FT cells transfected without or with SpRNase III respectively. DR-EMX1(1)-DR was processed into mature crRNAs only in the presence of SpCas9 and short tracrRNA, and was not dependent on the presence of SpRNase III. The mature crRNA detected from transfected 293FT total RNA is ˜33 bp and is shorter than the 39-42 bp mature crRNA from S. pyogenes (E. Deltcheva et al., Nature 471, 602 (Mar. 31, 2011)), suggesting that the processed mature crRNA in human 293FT cells is likely different from the bacterial mature crRNA in S. pyogenes. FIG. 30A discloses SEQ ID NO: 489.

FIG. 31A-B depict a bicistronic expression vectors for pre-crRNA array or chimeric crRNA with Cas9. (A) Schematic showing the design of an expression vector for the pre-crRNA array. Spacers can be inserted between two BbsI sites using annealed oligonucleotides. Sequence design for the oligonucleotides are shown below with the appropriate ligation adapters indicated. (B) Schematic of the expression vector for chimeric crRNA. The guide sequence can be inserted between two BbsI sites using annealed oligonucleotides. The vector already contains the partial direct repeat (gray) and partial tracrRNA (red) sequences. WPRE, Woodchuck hepatitis virus posttranscriptional regulatory element. FIG. 31A discloses SEQ ID NOS 490-492, and FIG. 31B discloses SEQ ID NOS 493-495, all respectively, in order of appearance.

FIGS. 32A-B depict a selection of protospacers in the human PVALB and mouse Th loci. Schematic of the human PVALB (A) and mouse Th (B) loci and the location of the three protospacers within the last exon of the PVALB and Th genes, respectively. The 30 bp protospacers are indicated by black lines and the adjacent PAM sequences are indicated by the magenta bar. Protospacers on the sense and anti-sense strands are indicated above and below the DNA sequences respectively. FIGS. 32A-B disclose SEQ ID NOS 496 and 497, respectively.

FIGS. 33A-C depict occurrences of PAM sequences in the human genome. Histograms of distances between adjacent Streptococcus pyogenes SF370 locus 1 PAM (NGG) (A) and Streptococcus thermophiles LMD9 locus 1 PAM (NNAGAAW) (B) in the human genome. (C) Distances for each PAM by chromosome. Chr, chromosome. Putative targets were identified using both the plus and minus strands of human chromosomal sequences. Given that there may be chromatin, DNA methylation-, RNA structure, and other factors that may limit the cleavage activity at some protospacer targets, it is important to note that the actual targeting ability might be less than the result of this computational analysis.

FIGS. 34A-D depict type II CRISPR from Streptococcus thermophilus LMD-9 can also function in eukaryotic cells. (A) Schematic of CRISPR locus 2 from Streptococcus thermophilus LMD-9. (B) Design of the expression system for the S. thermophilus CRISPR system. Human codon-optimized hStCas9 is expressed using a constitutive EF1a promoter. Mature versions of tracrRNA and crRNA are expressed using the U6 promoter to ensure precise transcription initiation. Sequences for the mature crRNA and tracrRNA are shown. A single based indicated by the lower case “a” in the crRNA sequence was used to remove the polyU sequence, which serves as a RNA Pol III transcriptional terminator. (C) Schematic showing protospacer and corresponding PAM sequences targets in the human EMX1 locus. Two protospacer sequences are highlighted and their corresponding PAM sequences satisfying the NNAGAAW motif are indicated by magenta lines. Both protospacers are targeting the anti-sense strand. (D) SURVEYOR assay showing StCas9-mediated cleavage in the target locus. RNA guide spacers 1 and 2 induced 14% and 6.4% respectively. Statistical analysis of cleavage activity across biological replica at these two protospacer sites can be found in Table 1. FIG. 34B discloses SEQ ID NOS 498-499, respectively, in order of appearance, and FIG. 34C discloses SEQ ID NO: 500.

FIG. 35 depicts an example of an AAV-promoter-TALE-effector construct, where hSyn=human synapsin 1 promoter, N+136=TALE N-term, AA+136 truncation, C63=TALE C-term, AA+63 truncation, vp=VP64 effector domain, GFP=green fluorescent protein, WPRE=Woodchuck Hepatitis Virus Posttranscriptional Regulatory Element, bGH=bovine growth hormone polyA, ITR=AAV inverted terminal repeat and AmpR=ampicillin resistance gene.

FIG. 36A-C depict design and optimization of the LITE system. (a) A TALE DNA-binding domain is fused to CRY2 and a transcriptional effector domain is fused to CIB1. In the inactive state, TALE-CRY2 binds the promoter region of the target gene while CIB1-effector remains unbound in the nucleus. The VP64 transcriptional activator is shown above. Upon illumination with blue light, TALE-CRY2 and CIB1-effector rapidly dimerize, recruiting CIB1-effector to the target promoter. The effector in turn modulates transcription of the target gene. (b) Light-dependent upregulation of the endogenous target Neurog2 mRNA with LITEs containing functional truncations of its light-sensitive binding partners. LITE-transfected Neuro-2a cells were stimulated for 24 h with 466 nm light at an intensity of 5 mW/cm² and a duty cycle of 7% (1 s pulses at 0.066 Hz). (c) Time course of light-dependent Neurog2 upregulation by TALE-CRY2PHR and CIB1-VP64 LITEs. LITE-transfected Neuro-2a cells were stimulated with 466 nm light at an intensity of 5 mW/cm² and a duty cycle of 7% (1 s pulses at 0.066 Hz) and decrease of Neurog2 mRNA levels after 6 h of light stimulation. All Neurog2 mRNA levels were measured relative to expressing GFP control cells (mean±s.e.m.; n=3-4) (*, p<0.05; and ***, p<0.001). FIG. 36A discloses SEQ ID NO: 20.

FIG. 37A-F depict in vitro and in vivo AAV-mediated TALE delivery targeting endogenous loci in neurons. (a) General schematic of constitutive TALE transcriptional activator packaged into AAV. Effector domain VP64 highlighted. hSyn: human synapsin promoter; 2A: foot-and-mouth disease-derived 2A peptide; WPRE: woodchuck hepatitis post-transcriptional response element; bGH pA: bovine growth hormone poly-A signal. (b) Representative images showing transduction with AAV-TALE-VP64 construct from (a) in primary cortical neurons. Cells were stained for GFP and neuronal marker NeuN. Scale bars=25 μm. (c) AAV-TALE-VP64 constructs targeting a variety of endogenous loci were screened for transcriptional activation in primary cortical neurons (*, p<0.05; **, p<0.01; ***, p<0.001). (d) Efficient delivery of TALE-VP64 by AAV into the ILC of mice. Scale bar=100 μm. (Cg1=cingulate cortex, PLC=prelimbic cortex, ILC=infralimbic cortex). (e) Higher magnification image of efficient transduction of neurons in ILC. (f) Grm2 mRNA upregulation by TALE-VP64 in vivo in ILC (mean±s.e.m.; n=3 animals per condition), measured using a 300 μm tissue punch.

FIGS. 38A-I depict LITE-mediated optogenetic modulation of endogenous transcription in primary neurons and in vivo. (a) AAV-LITE activator construct with switched CRY2PHR and CIB1 architecture. (b) Representative images showing co-transduction of AAV-delivered LITE constructs in primary neurons. Cells were stained for GFP, HA-tag, and DAPI. (Scale bars=25 m). (c) Light-induced activation of Grm2 expression in primary neurons after 24 h of stimulation with 0.8% duty cycle pulsed 466 nm light (250 ms pulses at 0.033 Hz or 500 ms pulses at 0.016 Hz; 5 mW/cm²). (d) Upregulation of Grm2 mRNA in primary cortical neurons with and without light stimulation at 4 h and 24 h time points. Expression levels are shown relative to neurons transduced with GFP only. (e) Quantification of mGluR2 protein levels in GFP only control transductions, unstimulated neurons with LITEs, and light-stimulated neurons with LITEs. A representative western blot is shown with β-tubulin-III as a loading control. (f) Schematic showing transduction of ILC with the LITE system, the optical fiber implant, and the 0.35 mm diameter brain punch used for tissue isolation. (g) Representative images of ILC co-transduced with both LITE components. Stains are shown for HA-tag (red), GFP (green), and DAPI (blue). (Scale bar=25 m). (h) Light-induced activation of endogenous Grm2 expression using LITEs transduced into ILC. **, p<0.05; data generated from 4 different mice for each experimental condition. (i) Fold increases and light induction of Neurog2 expression using LITE1.0 and optimized LITE 2.0. LITE2.0 provides minimal background while maintaining a high level of activation. NLS_{α-importin} and NLS_{SV40}, nuclear localization signal from α-importin and simian virus 40 respectively; GS, Gly-Ser linker; NLS*, mutated NLS where the indicated residues have been substituted with Ala to prevent nuclear localization activity; Δ318-334; deletion of a higher plant helix-loop-helix transcription factor homology region. FIG. 38I discloses SEQ ID NO: 501.

FIG. 39A-H depict TALE- and LITE-mediated epigenetic modifications (a) Schematic of LITE epigenetic modifiers (epiLITE). (b) Schematic of engineered epigenetic transcriptional repressor SID4X within an AAV vector. phiLOV2.1 (330 bp) was used as a fluorescent marker rather than GFP (800 bp) to ensure efficient AAV packaging. (c) epiLITE-mediated repression of endogenous Grm2 expression in primary cortical neurons with and without light stimulation. Fold down regulation is shown relative to neurons transduced with GFP alone. (d) epiLITE-mediated decrease in H3K9 histone residue acetylation at the Grm2 promoter with and without light-stimulation. (e, f) Fold reduction of Grm2 mRNA by epiTALE-methyltransferases (epiTALE-KYP, -TgSET8, and -NUE), and corresponding enrichment of histone methylation marks H3K9me1, H4K20me3, and H3K27me3 at the Grm2 promoter. (g, h) Fold reduction of Grm2 mRNA by epiTALE histone deacetylases (epiTALE-HDAC8, -RPD3, -Sir2a, and -Sin3a), and corresponding decreases in histone residue acetylation marks H4K8Ac and H3K9Ac at the Grm2 promoter. Values shown in all panels are mean±s.e.m., n=3-4.

FIG. 40 depicts an illustration of the absorption spectrum of CRY2 in vitro. Cryptochrome 2 was optimally activated by 350-475 nm light¹. A sharp drop in absorption and activation was seen for wavelengths greater than 480 nm. Spectrum was adapted from Banerjee, R. et al. The Signaling State of Arabidopsis Cryptochrome 2 Contains Flavin Semiquinone. Journal of Biological Chemistry 282, 14916-14922, doi:10.1074/jbc.M700616200 (2007).

FIG. 41 depicts an impact of illumination duty cycle on LITE-mediated gene expression. Varying duty cycles (illumination as percentage of total time) were used to stimulate 293FT cells expressing LITEs targeting the KLF4 gene, in order to investigate the effect of duty cycle on LITE activity. KLF4 expression levels were compared to cells expressing GFP only. Stimulation parameters were: 466 nm, 5 mW/cm² for 24 h. Pulses were performed at 0.067 Hz with the following durations: 1.7%=0.25 s pulse, 7%=1 s pulse, 27%=4 s pulse, 100%=constant illumination. (mean±s.e.m.; n=3-4).

FIGS. 42A-B depict an impact of light intensity on LITE-mediated gene expression and cell survival. (a) The transcriptional activity of CRY2PHR::CIB1 LITE was found to vary according to the intensity of 466 nm blue light. Neuro 2a cells were stimulated for 24 h hours at a 7% duty cycle (is pulses at 0.066 Hz) (b) Light-induced toxicity measured as the percentage of cells positive for red-fluorescent ethidium homodimer-1 versus calcein-positive cells. All Neurog2 mRNA levels were measured relative to cells expressing GFP only (mean±s.e.m.; n=3-4).

FIG. 43 depicts an impact of transcriptional activation domains on LITE-mediated gene expression. Neurog2 up-regulation with and without light by LITEs using different transcriptional activation domains (VP16, VP64, and p65). Neuro-2a cells transfected with LITE were stimulated for 24 h with 466 nm light at an intensity of 5 mW/cm² and a duty cycle of 7% (1 s pulses at 0.066 Hz). (mean±s.e.m.; n=3-4)

FIGS. 44A-C depict chemical induction of endogenous gene transcription. (a) Schematic showing the design of a chemical inducible two hybrid TALE system based on the abscisic acid (ABA) receptor system. ABI and PYL dimerize upon the addition of ABA and dissociates when ABA is withdrawn. (b) Time course of ABA-dependent Neurog2 up-regulation. 250 μM of ABA was added to HEK 293FT cells expressing TALE(Neurog2)-ABI and PYL-VP64. Fold mRNA increase was measured at the indicated time points after the addition of ABA. (c) Decrease of Neurog2 mRNA levels after 24 h of ABA stimulation. All Neurog2 mRNA levels were measured relative to expressing GFP control cells (mean±s.e.m.; n=3-4). FIG. 44A discloses SEQ ID NOS 27 and 27.

FIGS. 45A-C depict AAV supernatant production. (a) Lentiviral and AAV vectors carrying GFP were used to test transduction efficiency. (b) Primary embryonic cortical neurons were transduced with 300 and 250 μL supernatant derived from the same number of AAV or lentivirus-transfected 293FT cells. Representative images of GFP expression were collected at 7 d.p.i. Scale bars=50 m. (c) The depicted process was developed for the production of AAV supernatant and subsequent transduction of primary neurons. 293FT cells were transfected with an AAV vector carrying the gene of interest, the AAV1 serotype packaging vector (pAAV1), and helper plasmid (pDF6) using PEI. 48 h later, the supernatant was harvested and filtered through a 0.45 μm PVDF membrane. Primary neurons were then transduced with supernatant and remaining aliquots were stored at −80° C. Stable levels of AAV construct expression were reached after 5-6 days. AAV supernatant production following this process can be used for production of up to 96 different viral constructs in 96-well format (employed for TALE screen in neurons shown in FIG. 37C).

FIG. 46 depicts selection of TALE target sites guided by DNaseI-sensitive chromatin regions. High DNaseI sensitivity based on mouse cortical tissue data from ENCODE (http://genome.ucsc.edu) was used to identify open chromatin regions. The peak with the highest amplitude within the region 2 kb upstream of the transcriptional start site was selected for targeting. TALE binding targets were then picked within a 200 bp region at the center of the peak.

FIG. 47 depicts an impact of light duty cycle on primary neuron health. The effect of light stimulation on primary cortical neuron health was compared for duty cycles of 7%, 0.8%, and no light conditions. Calcein was used to evaluate neuron viability. Bright-field images were captured to show morphology and cell integrity. Primary cortical neurons were stimulated with the indicated duty cycle for 24 h with 5 mW/cm² of 466 nm light. Representative images, scale bar=50 μm. Pulses were performed in the following manner: 7% duty cycle=1 s pulse at 0.067 Hz, 0.8% duty cycle=0.5 s pulse at 0.0167 Hz.

FIG. 48 depicts an image of a mouse during optogenetic stimulation. An awake, freely behaving, LITE-injected mouse is pictured with a stereotactically implanted cannula and optical fiber.

FIG. 49 depicts co-transduction efficiency of LITE components by AAV1/2 in mouse infralimbic cortex. Cells transduced by TALE(Grm2)-CIB1 alone, CRY2PHR-VP64 alone, or co-transduced were calculated as a percentage of all transduced cells.

FIG. 50 depicts a contribution of individual LITE components to baseline transcription modulation. Grm2 mRNA levels were determined in primary neurons transfected with individual LITE components. Primary neurons expressing Grm2 TALE_1-CIB1 alone led to a similar increase in Grm2 mRNA levels as unstimulated cells expressing the complete LITE system. (mean±s.e.m.; n=3-4).

FIG. 51A-C depicts effects of LITE Component Engineering on Activation, Background Signal, and Fold Induction. Protein modifications were employed to find LITE components resulting in reduced background transcriptional activation while improving induction ratio by light. Protein alterations are discussed in detail below. In brief, nuclear localization signals and mutations in an endogenous nuclear export signal were used to improve nuclear import of the CRY2PHR-VP64 component. Several variations of CIB1 intended to either reduce nuclear localization or CIB1 transcriptional activation were pursued in order to reduce the contribution of the TALE-CIB1 component to background activity. The results of all combinations of CRY2PHR-VP64 and TALE-CIB1 which were tested are shown above. The table to the left of the bar graphs indicates the particular combination of domains/mutations used for each condition. Each row of the table and bar graphs contains the component details, Light/No light activity, and induction ratio by light for the particular CRY2PHR/CIB1 combination. Combinations that resulted in both decreased background and increased fold induction compared to LITE 1.0 are highlighted in green in the table column marked “+” (t-test p<0.05). CRY2PHR-VP64 Constructs: Three new constructs were designed with the goal of improving CRY2PHR-VP64 nuclear import. First, the mutations L70A and L74A within a predicted endogenous nuclear export sequence of CRY2PHR were induced to limit nuclear export of the protein (referred to as ‘*’ in the Effector column). Second, the α-importin nuclear localization sequence was fused to the N-terminus of CRY2PHR-VP64 (referred to as ‘A’ in the Effector column). Third, the SV40 nuclear localization sequence was fused to the C-terminus of CRY2PHR-VP64 (referred to as ‘P’ in the Effector column). TALE-CIB1 Linkers: The SV40 NLS linker between TALE and CIB1 used in LITE 1.0 was replaced with one of several linkers designed to increase nuclear export of the TALE-CIB1 protein (The symbols used in the CIB1 Linker column are shown in parentheses): a flexible glycine-serine linker (G), an adenovirus type 5 E1B nuclear export sequence (W), an HIV nuclear export sequence (M), a MAPKK nuclear export sequence (K), and a PTK2 nuclear export sequence (P). NLS* Endogenous CIB1 Nuclear Localization Sequence Mutation: A nuclear localization signal exists within the wild type CIB1 sequence. This signal was mutated in NLS* constructs at K92A, R93A, K105A, and K106A in order to diminish TALE-CIB1 nuclear localization (referred to as ‘N’ in the NLS* column). ΔCIB1 Transcription Factor Homology Deletions: In an effort to eliminate possible basal CIB1 transcriptional activation, deletion constructs were designed in which regions of high homology to basic helix-loop-helix transcription factors in higher plants were removed. These deleted regions consisted of Δaa230-256, Δaa276-307, Δaa308-334 (referred to as ‘1’ ‘2’ and ‘3’ in the ΔCIB1 column). In each case, the deleted region was replaced with a 3 residue GGS link. NES Insertions into CIB1: One strategy to facilitate light-dependent nuclear import of TALE-CIB1 was to insert an NES in CIB1 at its dimerization interface with CRY2PHR such that the signal would be concealed upon binding with CRY2PHR. To this end, an NES was inserted at different positions within the known CRY2 interaction domain CIBN (aa 1-170). The positions are as follows (The symbols used in the NES column are shown in parentheses): aa28 (1), aa52 (2), aa73 (3), aa120 (4), aa140 (5), aa160 (6). *bHLH basic Helix-Loop-Helix Mutation: To reduce direct CIB1-DNA interactions, several basic residues of the basic helix-loop-helix region in CIB1 were mutated. The following mutations are present in all *bHLH constructs (referred to as ‘B’ in the *bHLH column of FIG. 51): R175A, G176A, R187A, and R189A. FIG. 51 discloses SEQ ID NOS 502, 501, and 503-504, respectively, in order of appearance.

FIG. 52A-B depicts an illustration of light mediated co-dependent nuclear import of TALE-CIB1 (a) In the absence of light, the TALE-CIB1 LITE component resides in the cytoplasm due to the absence of a nuclear localization signal, NLS (or the addition of a weak nuclear export signal, NES). The CRY2PHR-VP64 component containing a NLS on the other hand is actively imported into the nucleus on its own. (b) In the presence of blue light, TALE-CIB1 binds to CRY2PHR. The strong NLS present in CRY2PHR-VP64 now mediates nuclear import of the complex of both LITE components, enabling them to activate transcription at the targeted locus.

FIG. 53 depicts notable LITE 1.9 combinations. In addition to the LITE 2.0 constructs, several CRY2PHR-VP64::TALE-CIB1 combinations from the engineered LITE component screen were of particular note. LITE 1.9.0, which combined the α-importin NLS effector construct with a mutated endogenous NLS and A276-307 TALE-CIB1 construct, exhibited an induction ratio greater than 9 and an absolute light activation of more than 180. LITE 1.9.1, which combined the unmodified CRY2PHR-VP64 with a mutated NLS, A318-334, AD5 NES TALE-CIB1 construct, achieved an induction ratio of 4 with a background activation of 1.06. A selection of other LITE 1.9 combinations with background activations lower than 2 and induction ratios ranging from 7 to 12 were also highlighted.

FIGS. 54A-D depict TALE SID4X repressor characterization and application in neurons. a) A synthetic repressor was constructed by concatenating 4 SID domains (SID4X). To identify the optimal TALE-repressor architecture, SID or SID4X was fused to a TALE designed to target the mouse p11 gene. (b) Fold decrease in p11 mRNA was assayed using qRT-PCR. (c) General schematic of constitutive TALE transcriptional repressor packaged into AAV. Effector domain SID4X is highlighted. hSyn: human synapsin promoter; 2A: foot-and-mouth disease-derived 2A peptide; WPRE: woodchuck hepatitis post-transcriptional response element; bGH pA: bovine growth hormone poly-A signal. phiLOV2.1 (330 bp) was chosen as a shorter fluorescent marker to ensure efficient AAV packaging. (d) 2 TALEs targeting the endogenous mouse loci Grm5, and Grm2 were fused to SID4X and virally transduced into primary neurons. The target gene down-regulation via SID4X is shown for each TALE relative to levels in neurons expressing GFP only. (mean±s.e.m.; n=3-4). FIG. 54A discloses SEQ ID NO: 450.

FIGS. 55A-B depict a diverse set of epiTALEs mediate transcriptional repression in neurons and Neuro2a cells a) A total of 24 Grm2 targeting TALEs fused to different histone effector domains were transduced into primary cortical mouse neurons using AAV. Grm2 mRNA levels were measured using RT-qPCR relative to neurons transduced with GFP only. * denotes repression with p<0.05. b) A total of 32 epiTALEs were transfected into Neuro2A cells. 20 of them mediated significant repression of the targeted Neurog2 locus (*=p<0.05).

FIGS. 56A-D depict epiTALEs mediating transcriptional repression along with histone modifications in Neuro 2A cells (a) TALEs fused to histone deacetylating epigenetic effectors NcoR and SIRT3 targeting the murine Neurog2 locus in Neuro 2A cells were assayed for repressive activity on Neurog2 transcript levels. (b) ChIP RT-qPCR showing a reduction in H3K9 acetylation at the Neurog2 promoter for NcoR and SIRT3 epiTALEs. (c) The epigenetic effector PHF19 with known histone methyltransferase binding activity was fused to a TALE targeting Neurog2 mediated repression of Neurog2 mRNA levels. (d) ChIP RT-qPCR showing an increase in H3K27me3 levels at the Neurog2 promoter for the PHF19 epiTALE.

FIGS. 57A-G depict RNA-guided DNA binding protein Cas9 can be used to target transcription effector domains to specific genomic loci. (a) The RNA-guided nuclease Cas9 from the type II Streptococcus pyogenes CRISPR/Cas system can be converted into a nucleolytically-inactive RNA-guided DNA binding protein (Cas9**) by introducing two alanine substitutions (D10A and H840A). Schematic showing that a synthetic guide RNA (sgRNA) can direct Cas9**-effector fusion to a specific locus in the human genome. The sgRNA contains a 20 bp guide sequence at the 5′ end which specifies the target sequence. On the target genomic DNA, the 20 bp target site needs to be followed by a 5′-NGG PAM motif. (b, c) Schematics showing the sgRNA target sites in the human KLF4 and SOX2 loci respectively. Each target site is indicated by the blue bar and the corresponding PAM sequence is indicated by the magenta bar. (d, e) Schematics of the Cas9**-VP64 transcription activator and SID4X-Cas9** transcription repressor constructs. (f, g) Cas9**-VP64 and SID4X-Cas9** mediated activation of KLF4 and repression of SOX2 respectively. All mRNA levels were measured relative to GFP mock transfected control cells (mean±s.e.m.; n=3). FIG. 57A discloses SEQ ID NOS 508-509, FIG. 57B discloses SEQ ID NO: 510, and FIG. 57C discloses SEQ ID NOS 511-513, all respectively, in order of appearance.

FIG. 58 depicts 6 TALEs which were designed, with two TALEs targeting each of the endogenous mouse loci Grm5, Grin2a, and Grm2. TALEs were fused to the transcriptional activator domain VP64 or the repressor domain SID4X and virally transduced into primary neurons. Both the target gene upregulation via VP64 and downregulation via SID4X are shown for each TALE relative to levels in neurons expressing GFP only. FIG. 58 discloses SEQ ID NOS 127, 505, 129, 506, 507, and 126, respectively, in order of appearance.

FIGS. 59A-B depict (A) LITE repressor construct highlighting SID4X repressor domain. (B) Light-induced repression of endogenous Grm2 expression in primary cortical neurons using Grm2 T1-LITE and Grm2 T2-LITE. Fold downregulation is shown relative to neurons transduced with GFP only (mean±s.e.m.; n=3-4 for all subpanels).

FIGS. 60A-B depict exchanging CRY2PHR and CIB1 components. (A) TALE-CIB1::CRY2PHR-VP64 was able to activate Ngn2 at higher levels than TALE-CRY2PHR::CIB1-VP64. (B) Fold activation ratios (light versus no light) ratios of Ngn2 LITEs show similar efficiency for both designs. Stimulation parameters were the same as those used in FIG. 36B.

FIG. 61 depicts Tet Cas9 vector designs for inducible Cas9.

FIG. 62 depicts a vector and EGFP expression in 293FT cells after Doxycycline induction of Cas9 and EGFP.

FIG. 63A-F illustrates an exemplary CRISPR system, a possible mechanism of action, an example adaptation for expression in eukmyotic cells, and results of tests assessing nuclear localization and CRISPR activity. FIG. 63 discloses SEQ ID NOS 544-553, respectively, in order of appearance.

FIG. 64A-C illustrates an exemplary expression cassette for expression of CRISPR system elements in eukaryotic cells, predicted structures of example guide sequences, and CRISPR system activity as measured in eukaryotic and prokaryotic cells. FIG. 64 discloses SEQ ID NOS 554-563, respectively, in order of appearance.

FIG. 65 provides a table of protospacer sequences and summarizes modification efficiency results for protospacer targets designed based on exemplary S. pyogenes and S. thermophilus CRISPR systems with corresponding PAMs against loci in human and mouse genomes. Cells were transfected with Cas9 and either pre-crRNA/tracrRNA or chimeric RNA, and analyzed 72 hours after transfection. Percent indels are calculated based on Surveyor assay results from indicated cell lines (N=3 for all protospacer targets, errors are S.E.M., N.D. indicates not detectable using the Surveyor assay, and N.T. indicates not tested in this study). FIG. 65 discloses SEQ ID NOS 564-579, respectively, in order of appearance.

FIG. 66A-D illustrates a bacterial plasmid transformation interference assay, expression cassettes and plasmids used therein, and transformation efficiencies of cells used therein. FIG. 66 discloses SEQ ID NOS 580-582, respectively, in order of appearance.

FIG. 67A-D illustrates an exemplary CRISPR system, an example adaptation for expression in eukaryotic cells, and results of tests assessing CRISPR activity. FIG. 67 discloses SEQ ID NOS 583-586, respectively, in order of appearance.

FIG. 68 provides a table of sequences for primers and probes used for Surveyor, RFLP, genomic sequencing, and Northern blot assays. FIG. 68 discloses SEQ ID NOS 587-589, respectively, in order of appearance.

### DETAILED DESCRIPTION OF THE INVENTION

The term “nucleic acid” or “nucleic acid sequence” refers to a deoxyribonucleic or ribonucleic oligonucleotide in either single- or double-stranded form. The term encompasses nucleic acids, i.e., oligonucleotides, containing known analogues of natural nucleotides. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” encompasses the ligation of nucleic acids having various coding regions or domains or promoter sequences from different sources into an expression cassette or vector for expression of, e.g., inducible or constitutive expression of polypeptide coding sequences in the vectors of invention.

The term “heterologous” when used with reference to a nucleic acid, indicates that the nucleic acid is in a cell or a virus where it is not normally found in nature; or, comprises two or more subsequences that are not found in the same relationship to each other as normally found in nature, or is recombinantly engineered so that its level of expression, or physical relationship to other nucleic acids or other molecules in a cell, or structure, is not normally found in nature. A similar term used in this context is “exogenous”. For instance, a heterologous nucleic acid is typically recombinantly produced, having two or more sequences from unrelated genes arranged in a manner not found in nature; e.g., a human gene operably linked to a promoter sequence inserted into an adenovirus-based vector of the invention. As an example, a heterologous nucleic acid of interest may encode an immunogenic gene product, wherein the adenovirus is administered therapeutically or prophylactically as a carrier or drug-vaccine composition. Heterologous sequences may comprise various combinations of promoters and sequences, examples of which are described in detail herein.

A “therapeutic ligand” may be a substance which may bind to a receptor of a target cell with therapeutic effects.

A “therapeutic effect” may be a consequence of a medical treatment of any kind, the results of which are judged by one of skill in the field to be desirable and beneficial. The “therapeutic effect” may be a behavioral or physiologic change which occurs as a response to the medical treatment. The result may be expected, unexpected, or even an unintended consequence of the medical treatment. A “therapeutic effect” may include, for example, a reduction of symptoms in a subject suffering from infection by a pathogen.

A “target cell” may be a cell in which an alteration in its activity may induce a desired result or response. As used herein, a cell may be an in vitro cell. The cell may be an isolated cell which may not be capable of developing into a complete organism.

A “ligand” may be any substance that binds to and forms a complex with a biomolecule to serve a biological purpose. As used herein, “ligand” may also refer to an “antigen” or “immunogen”. As used herein “antigen” and “immunogen” are used interchangeably.

“Expression” of a gene or nucleic acid encompasses not only cellular gene expression, but also the transcription and translation of nucleic acid(s) in cloning systems and in any other context.

As used herein, a “vector” is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. The present invention comprehends recombinant vectors that may include viral vectors, bacterial vectors, protozoan vectors, DNA vectors, or recombinants thereof.

With respect to exogenous DNA for expression in a vector (e.g., encoding an epitope of interest and/or an antigen and/or a therapeutic) and documents providing such exogenous DNA, as well as with respect to the expression of transcription and/or translation factors for enhancing expression of nucleic acid molecules, and as to terms such as “epitope of interest”, “therapeutic”, “immune response”, “immunological response”, “protective immune response”, “immunological composition”, “immunogenic composition”, and “vaccine composition”, inter alia, reference is made to U.S. Pat. No. 5,990,091 issued Nov. 23, 1999, and WO 98/00166 and WO 99/60164, and the documents cited therein and the documents of record in the prosecution of that patent and those PCT applications; all of which are incorporated herein by reference. Thus, U.S. Pat. No. 5,990,091 and WO 98/00166 and WO 99/60164 and documents cited therein and documents of record in the prosecution of that patent and those PCT applications, and other documents cited herein or otherwise incorporated herein by reference, may be consulted in the practice of this invention; and, all exogenous nucleic acid molecules, promoters, and vectors cited therein may be used in the practice of this invention. In this regard, mention is also made of U.S. Pat. Nos. 6,706,693; 6,716,823; 6,348,450; U.S. patent application Ser. Nos. 10/424,409; 10/052,323; 10/116,963; 10/346,021; and WO 99/08713, published Feb. 25, 1999, from PCT/US98/16739.

Aspects of the invention comprehend the TALE and CRISPR-Cas systems of the invention being delivered into an organism or a cell or to a locus of interest via a delivery system. One means of delivery is via a vector, wherein the vector is a viral vector, such as a lenti- or baculo- or preferably adeno-viral/adeno-associated viral vectors, but other means of delivery are known (such as yeast systems, microvesicles, gene guns/means of attaching vectors to gold nanoparticles) and are provided. In some embodiments, one or more of the viral or plasmid vectors may be delivered via nanoparticles, exosomes, microvesciles, or a gene-gun.

As used herein, the terms “drug composition” and “drug”, “vaccinal composition”, “vaccine”, “vaccine composition”, “therapeutic composition” and “therapeutic-immunologic composition” cover any composition that induces protection against an antigen or pathogen. In some embodiments, the protection may be due to an inhibition or prevention of infection by a pathogen. In other embodiments, the protection may be induced by an immune response against the antigen(s) of interest, or which efficaciously protects against the antigen; for instance, after administration or injection into the subject, elicits a protective immune response against the targeted antigen or immunogen or provides efficacious protection against the antigen or immunogen expressed from the inventive adenovirus vectors of the invention. The term “pharmaceutical composition” means any composition that is delivered to a subject. In some embodiments, the composition may be delivered to inhibit or prevent infection by a pathogen.

A “therapeutically effective amount” is an amount or concentration of the recombinant vector encoding the gene of interest, that, when administered to a subject, produces a therapeutic response or an immune response to the gene product of interest.

The term “viral vector” as used herein includes but is not limited to retroviruses, adenoviruses, adeno-associated viruses, alphaviruses, and herpes simplex virus.

The term“polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non nucleotide components. A polynucleotide may be further modified after polymerization, such as by conjugation with a labeling component.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The present invention comprehends spatiotemporal control of endogenous or exogenous gene expression using a form of energy. The form of energy may include but is not limited to electromagnetic radiation, sound energy, chemical energy and thermal energy. In a preferred embodiment of the invention, the form of energy is electromagnetic radiation, preferably, light energy. Previous approaches to control expression of endogenous genes, such as transcription activators linked to DNA binding zinc finger proteins provided no mechanism for temporal or spatial control. The capacity for photoactivation of the system described herein allows the induction of gene expression modulation to begin at a precise time within a localized population of cells.

Aspects of control as detailed in this application relate to at least one or more switch(es). The term “switch” as used herein refers to a system or a set of components that act in a coordinated manner to affect a change, encompassing all aspects of biological function such as activation, repression, enhancement or termination of that function. In one aspect the term switch encompasses genetic switches which comprise the basic components of gene regulatory proteins and the specific DNA sequences that these proteins recognize. In one aspect, switches relate to inducible and repressible systems used in gene regulation. In general, an inducible system may be off unless there is the presence of some molecule (called an inducer) that allows for gene expression. The molecule is said to “induce expression”. The manner by which this happens is dependent on the control mechanisms as well as differences in cell type. A repressible system is on except in the presence of some molecule (called a corepressor) that suppresses gene expression. The molecule is said to “repress expression”. The manner by which this happens is dependent on the control mechanisms as well as differences in cell type. The term “inducible” as used herein may encompass all aspects of a switch irrespective of the molecular mechanism involved. Accordingly a switch as comprehended by the invention may include but is not limited to antibiotic based inducible systems, electromagnetic energy based inducible systems, small molecule based inducible systems, nuclear receptor based inducible systems and hormone based inducible systems. In preferred embodiments the switch may be a tetracycline (Tet)/DOX inducible system, a light inducible systems, a Abscisic acid (ABA) inducible system, a cumate repressor/operator system, a 4OHT/estrogen inducible system, an ecdysone-based inducible systems or a FKBP12/FRAP (FKBP12-rapamycin complex) inducible system.

In one aspect of the invention at least one switch may be associated with a TALE or CRISPR-Cas system wherein the activity of the TALE or CRISPR-Cas system is controlled by contact with at least one inducer energy source as to the switch. The term “contact” as used herein for aspects of the invention refers to any associative relationship between the switch and the inducer energy source, which may be a physical interaction with a component (as in molecules or proteins which bind together) or being in the path or being struck by energy emitted by the energy source (as in the case of absorption or reflection of light, heat or sound). In some aspects of the invention the contact of the switch with the inducer energy source is brought about by application of the inducer energy source. The invention also comprehends contact via passive feedback systems. This includes but is not limited to any passive regulation mechanism by which the TALE or CRISPR-Cas system activity is controlled by contact with an inducer energy source that is already present and hence does not need to be applied. For example this energy source may be a molecule or protein already existent in the cell or in the cellular environment. Interactions which bring about contact passively may include but are not limited to receptor/ligand binding, receptor/chemical ligand binding, receptor/protein binding, antibody/protein binding, protein dimerization, protein heterodimerization, protein multimerization, nuclear receptor/ligand binding, post-translational modifications such as phosphorylation, dephosphorylation, ubiquitination or deubiquitination.

Two key molecular tools were leveraged in the design of the photoresponsive transcription activator-like (TAL) effector system. First, the DNA binding specificity of engineered TAL effectors is utilized to localize the complex to a particular region in the genome. Second, light-induced protein dimerization is used to attract an activating or repressing domain to the region specified by the TAL effector, resulting in modulation of the downstream gene.

Inducible effectors are contemplated for in vitro or in vivo application in which temporally or spatially specific gene expression control is desired. In vitro examples: temporally precise induction/suppression of developmental genes to elucidate the timing of developmental cues, spatially controlled induction of cell fate reprogramming factors for the generation of cell-type patterned tissues. In vivo examples: combined temporal and spatial control of gene expression within specific brain regions.

In a preferred embodiment of the invention, the inducible effector is a Light Inducible Transcriptional Effector (LITE). The modularity of the LITE system allows for any number of effector domains to be employed for transcriptional modulation. In a particularly advantageous embodiment, transcription activator like effector (TALE) and the activation domain VP64 are utilized in the present invention.

LITEs are designed to modulate or alter expression of individual endogenous genes in a temporally and spatially precise manner. Each LITE may comprise a two component system consisting of a customized DNA-binding transcription activator like effector (TALE) protein, a light-responsive cryptochrome heterodimer from Arabadopsis thaliana, and a transcriptional activation/repression domain. The TALE is designed to bind to the promoter sequence of the gene of interest. The TALE protein is fused to one half of the cryptochrome heterodimer (cryptochrome-2 or CIB1), while the remaining cryptochrome partner is fused to a transcriptional effector domain. Effector domains may be either activators, such as VP16, VP64, or p65, or repressors, such as KRAB, EnR, or SID. In a LITE's unstimulated state, the TALE-cryptochrome2 protein localizes to the promoter of the gene of interest, but is not bound to the CIB1-effector protein. Upon stimulation of a LITE with blue spectrum light, cryptochrome-2 becomes activated, undergoes a conformational change, and reveals its binding domain. CIB1, in turn, binds to cryptochrome-2 resulting in localization of the effector domain to the promoter region of the gene of interest and initiating gene overexpression or silencing.

Activator and repressor domains may selected on the basis of species, strength, mechanism, duration, size, or any number of other parameters. Preferred effector domains include, but are not limited to, a transposase domain, integrase domain, recombinase domain, resolvase domain, invertase domain, protease domain, DNA methyltransferase domain, DNA demethylase domain, histone acetylase domain, histone deacetylases domain, nuclease domain, repressor domain, activator domain, nuclear-localization signal domains, transcription-protein recruiting domain, cellular uptake activity associated domain, nucleic acid binding domain or antibody presentation domain.

Gene targeting in a LITE or in any other inducible effector may be achieved via the specificity of customized TALE DNA binding proteins. A target sequence in the promoter region of the gene of interest is selected and a TALE customized to this sequence is designed. The central portion of the TALE consists of tandem repeats 34 amino acids in length. Although the sequences of these repeats are nearly identical, the 12th and 13th amino acids (termed repeat variable diresidues) of each repeat vary, determining the nucleotide-binding specificity of each repeat. Thus, by synthesizing a construct with the appropriate ordering of TALE monomer repeats, a DNA binding protein specific to the target promoter sequence is created.

In advantageous embodiments of the invention, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. A general representation of a TALE monomer which is comprised within the DNA binding domain is X₁₋₁₁-(X₁₂X₁₃)-X_{14-33 or 34 or 35}, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X_{14-33 or 34 or 35})_{z}, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), monomers with an RVD of NG preferentially bind to thymine (T), monomers with an RVD of HD preferentially bind to cytosine (C) and monomers with an RVD of NN preferentially bind to both adenine (A) and guanine (G). In yet another embodiment of the invention, monomers with an RVD of IG preferentially bind to T. Thus, the number and order of the polypeptide monomer repeats in the nucleic acid binding domain of a TALE determines its nucleic acid target specificity. In still further embodiments of the invention, monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.
The polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, monomers having RVDs of H*, HA, KA, N*, NA, NC, NS, RA, and S* bind to adenine, guanine, cytosine and thymine with comparable affinity.

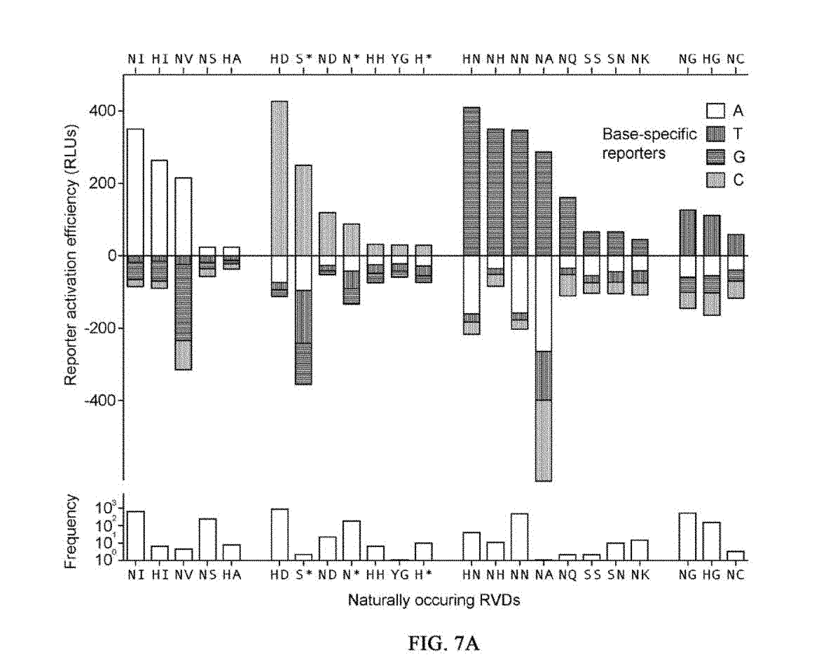

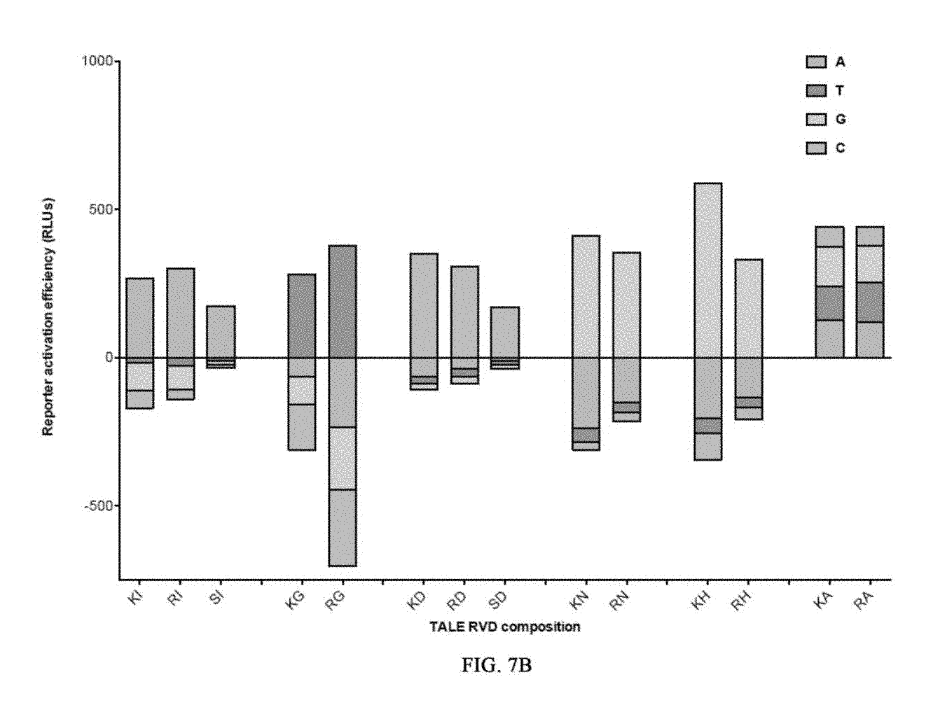

In even more advantageous embodiments of the invention the RVDs that have a specificity for adenine are NI, RI, KI, HI, and SI. In more preferred embodiments of the invention, the RVDs that have a specificity for adenine are HN, SI and RI, most preferably the RVD for adenine specificity is SI. In even more preferred embodiments of the invention the RVDs that have a specificity for thymine are NG, HG, RG and KG. In further advantageous embodiments of the invention, the RVDs that have a specificity for thymine are KG, HG and RG, most preferably the RVD for thymine specificity is KG or RG. In even more preferred embodiments of the invention the RVDs that have a specificity for cytosine are HD, ND, KD, RD, HH, YG and SD. In a further advantageous embodiment of the invention, the RVDs that have a specificity for cytosine are SD and RD. Refer to FIG. 7B for representative RVDs and the nucleotides they target to be incorporated into the most preferred embodiments of the invention. In a further advantageous embodiment the variant TALE monomers may comprise any of the RVDs that exhibit specificity for a nucleotide as depicted in FIG. 7A. All such TALE monomers allow for the generation of degenerative TALE polypeptides able to bind to a repertoire of related, but not identical, target nucleic acid sequences. In still further embodiments of the invention, the RVD NT may bind to G and A. In yet further embodiments of the invention, the RVD NP may bind to A, T and C. In more advantageous embodiments of the invention, at least one selected RVD may be NI, HD, NG, NN, KN, RN, NH, NQ, SS, SN, NK, KH, RH, HH, KI, HI, RI, SI, KG, HG, RG, SD, ND, KD, RD, YG, HN, NV, NS, HA, S*, N*, KA, H*, RA, NA or NC.

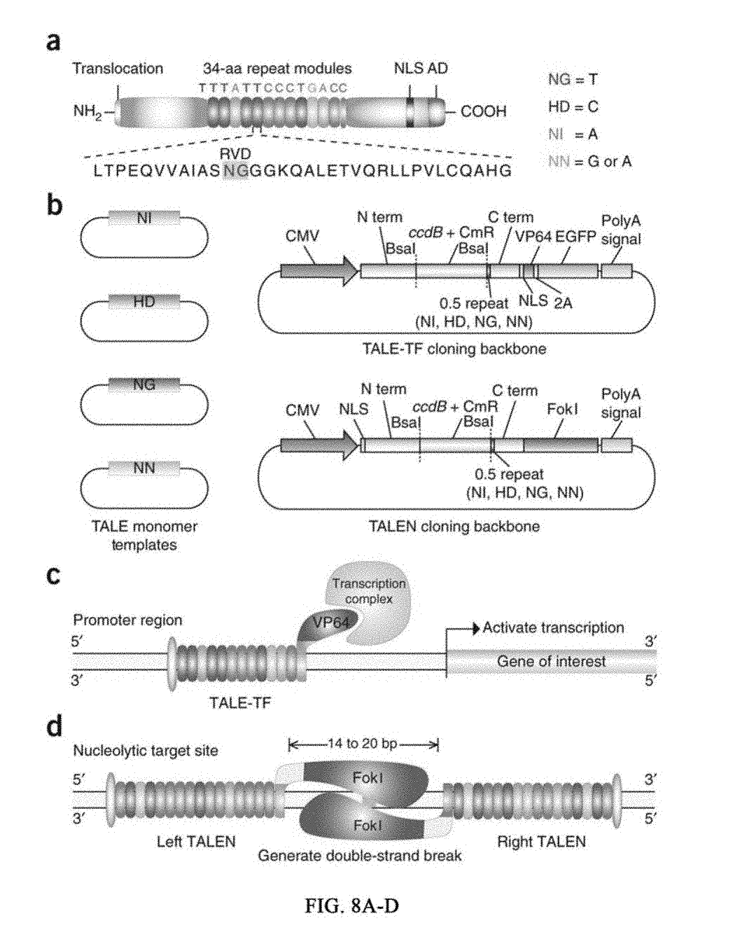

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8). Therefore, it follows that the length of the nucleic acid or DNA being targeted is equal to the number of full monomers plus two.

For example, nucleic acid binding domains may be engineered to contain 5, 6, 7, 8, 9, 10, 11, 12, 13, 14, 15, 16, 17, 18, 19, 20, 21, 22, 23, 24, 25, or more polypeptide monomers arranged in a N-terminal to C-terminal direction to bind to a predetermined 5, 6, 7, 8, 9, 10, 11, 12, 13, 14, 15, 16, 17, 18, 19, 20, 21, 22, 23, 24, 25 nucleotide length nucleic acid sequence. In more advantageous embodiments of the invention, nucleic acid binding domains may be engineered to contain 5, 6, 7, 8, 9, 10, 11, 12, 13, 14, 15, 16, 17, 18, 19, 20, 21, 22, 23, 24, 25, 26 or more full length polypeptide monomers that are specifically ordered or arranged to target nucleic acid sequences of length 7, 8, 9, 10, 11, 12, 13, 14, 15, 16, 17, 18, 19, 20, 21, 22, 23, 24, 25, 26, 27 and 28 nucleotides, respectively. In certain embodiments the polypeptide monomers are contiguous. In some embodiments, half-monomers may be used in the place of one or more monomers, particularly if they are present at the C-terminus of the TALE polypeptide.

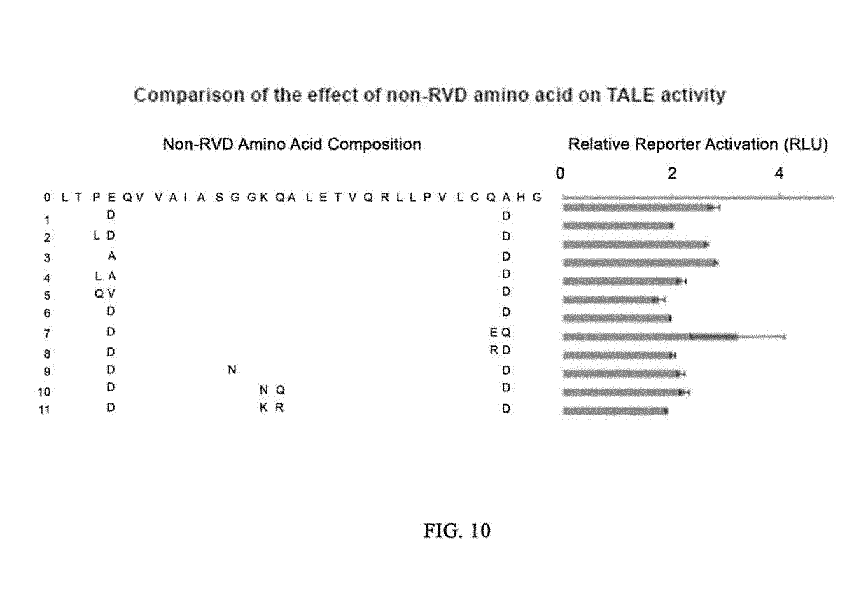

Polypeptide monomers are generally 33, 34 or 35 amino acids in length. With the exception of the RVD, the amino acid sequences of polypeptide monomers are highly conserved or as described herein, the amino acids in a polypeptide monomer, with the exception of the RVD, exhibit patterns that effect TALE activity, the identification of which may be used in preferred embodiments of the invention. Representative combinations of amino acids in the monomer sequence, excluding the RVD, are shown by the Applicants to have an effect on TALE activity (FIG. 10). In more preferred embodiments of the invention, when the DNA binding domain comprises (X₁₋₁₁-X₁₂X₁₃-X_{14-33 or 34 or 35})_{z}, wherein X₁₋₁₁ is a chain of 11 contiguous amino acids, wherein X₁₂X₁₃ is a repeat variable diresidue (RVD), wherein X₁₄₋₃₃ or 34 or 35 is a chain of 21, 22 or 23 contiguous amino acids, wherein z is at least 5 to 26, then the preferred combinations of amino acids are [LTLD] (SEQ ID NO: 1) or [LTLA] (SEQ ID NO: 2) or [LTQV] (SEQ ID NO: 3) at X₁₋₄, or [EQHG] (SEQ ID NO: 4) or [RDHG] (SEQ ID NO: 5) at positions X₃₀₋₃₃ or X₃₁₋₃₄ or X₃₂₋₃₅. Furthermore, other amino acid combinations of interest in the monomers are [LTPD] (SEQ ID NO: 7) at X₁₋₄ and [NQALE] (SEQ ID NO: 8) at X₁₆₋₂₀ and [DHG] at X₃₂₋₃₄ when the monomer is 34 amino acids in length. When the monomer is 33 or 35 amino acids long, then the corresponding shift occurs in the positions of the contiguous amino acids [NQALE] (SEQ ID NO: 8) and [DHG]; preferably, embodiments of the invention may have [NQALE] (SEQ ID NO: 8) at X₁₅₋₁₉ or X₁₇₋₂₁ and [DHG] at X₃₁₋₃₃ or X₃₃₋₃₅.

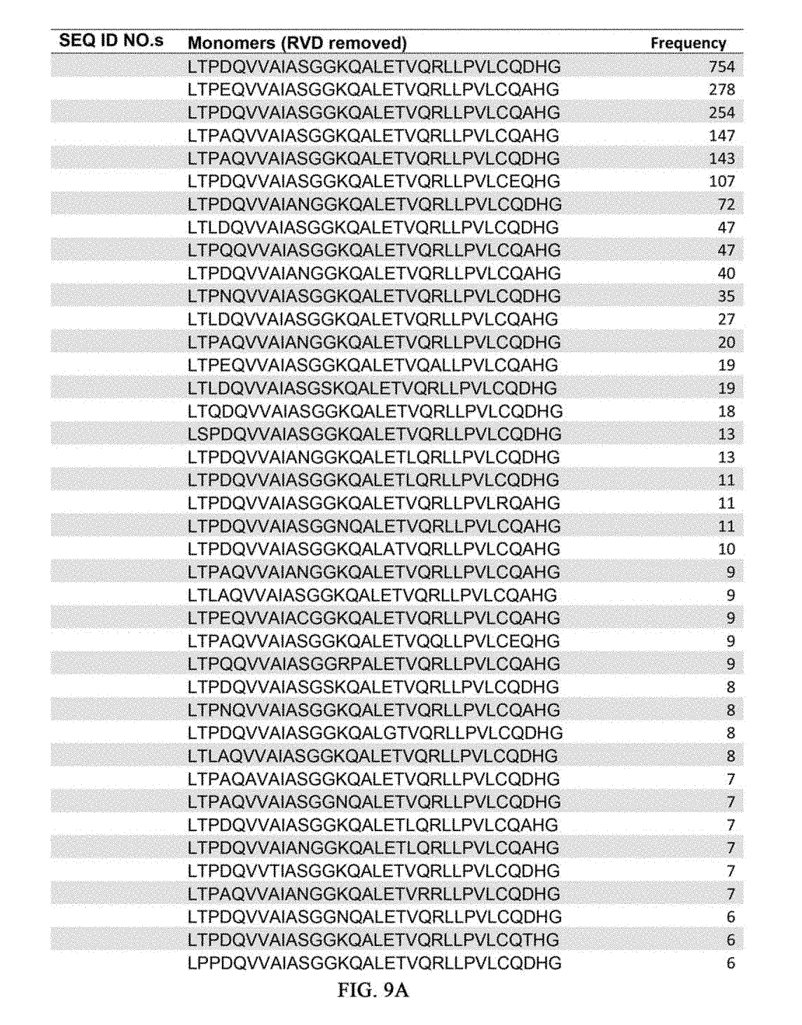

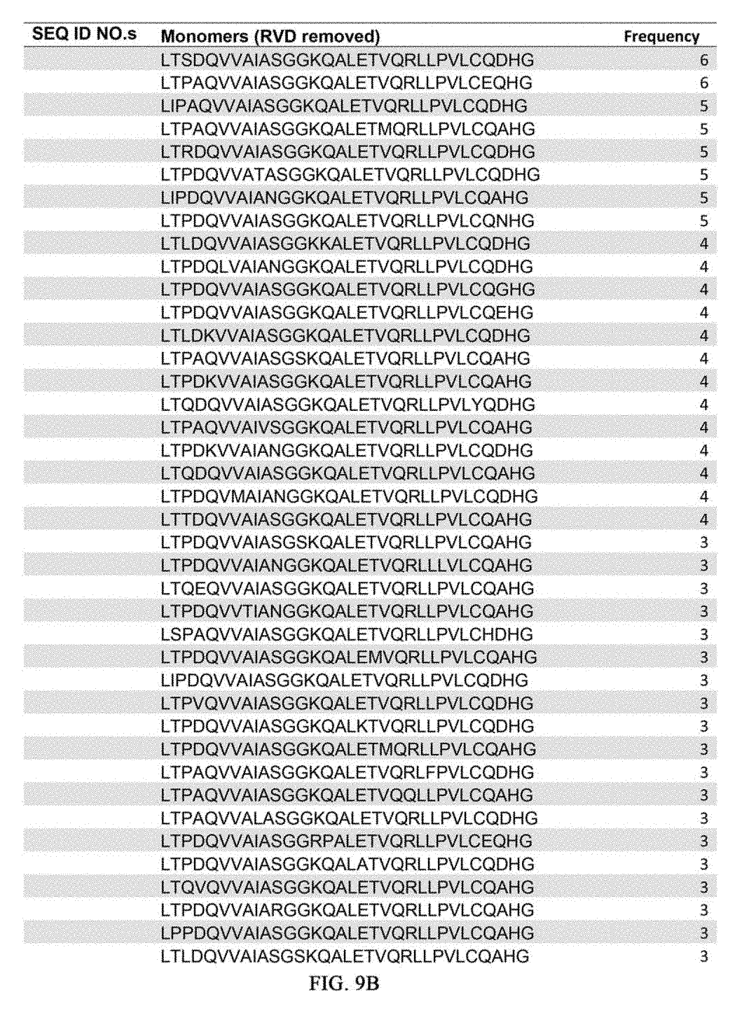

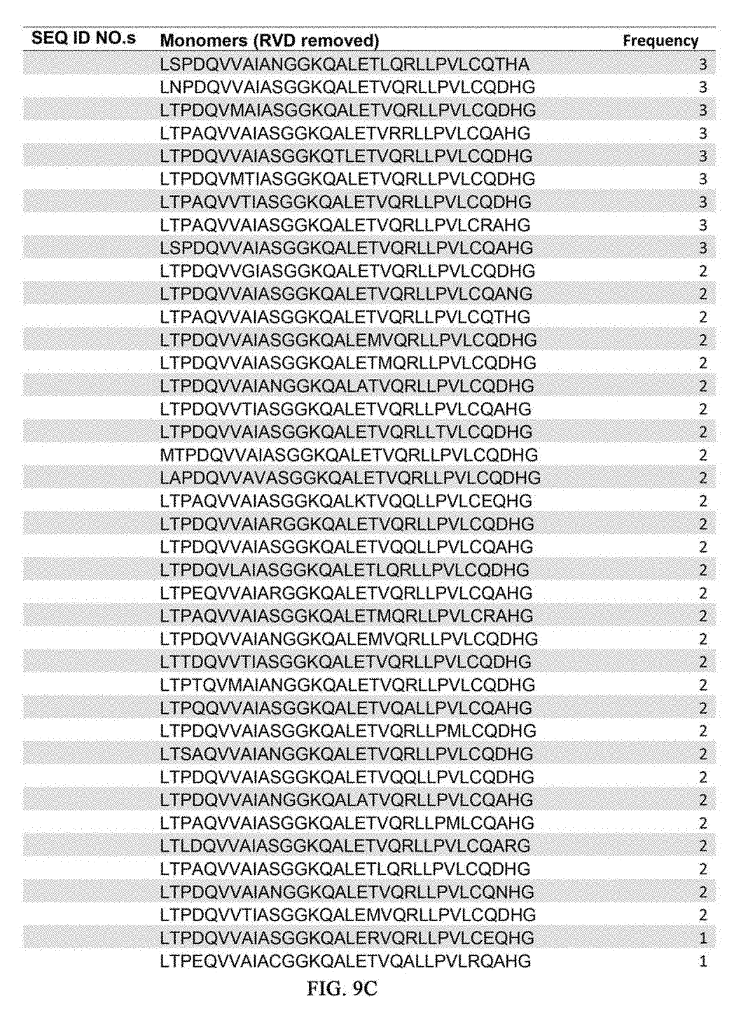

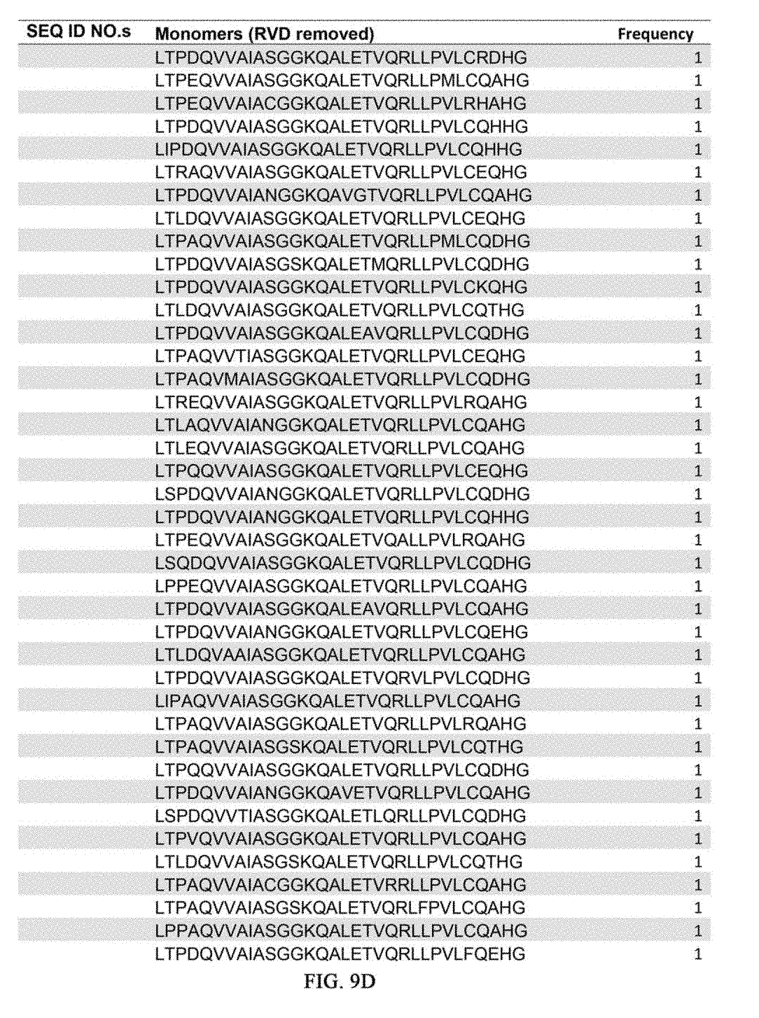

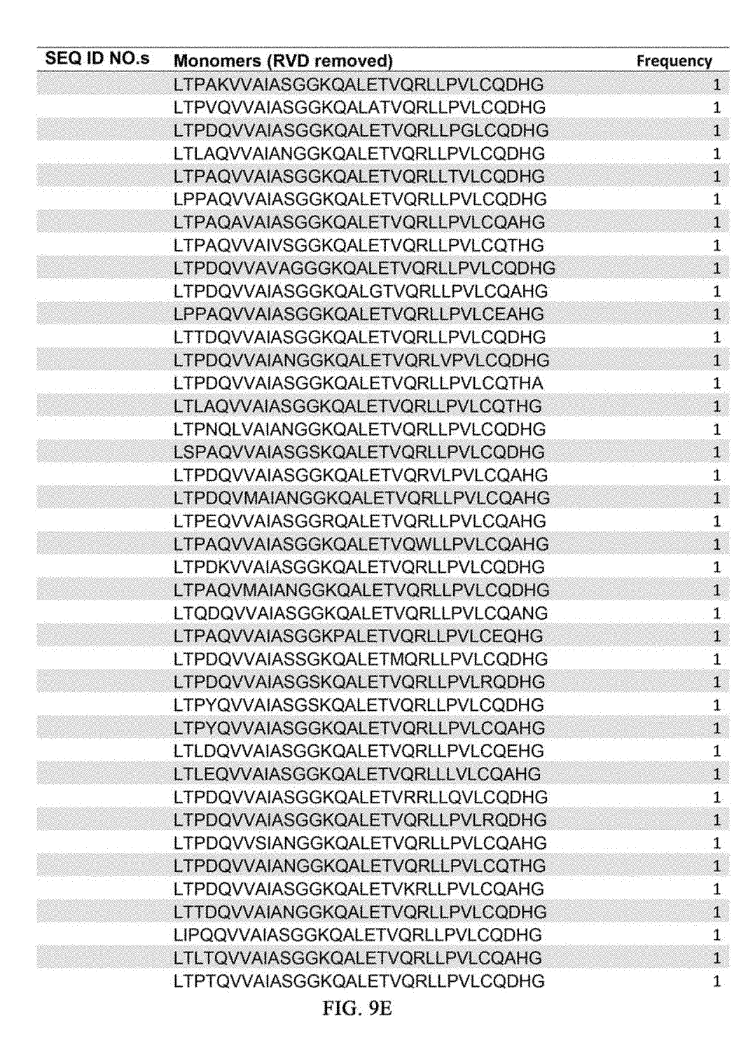

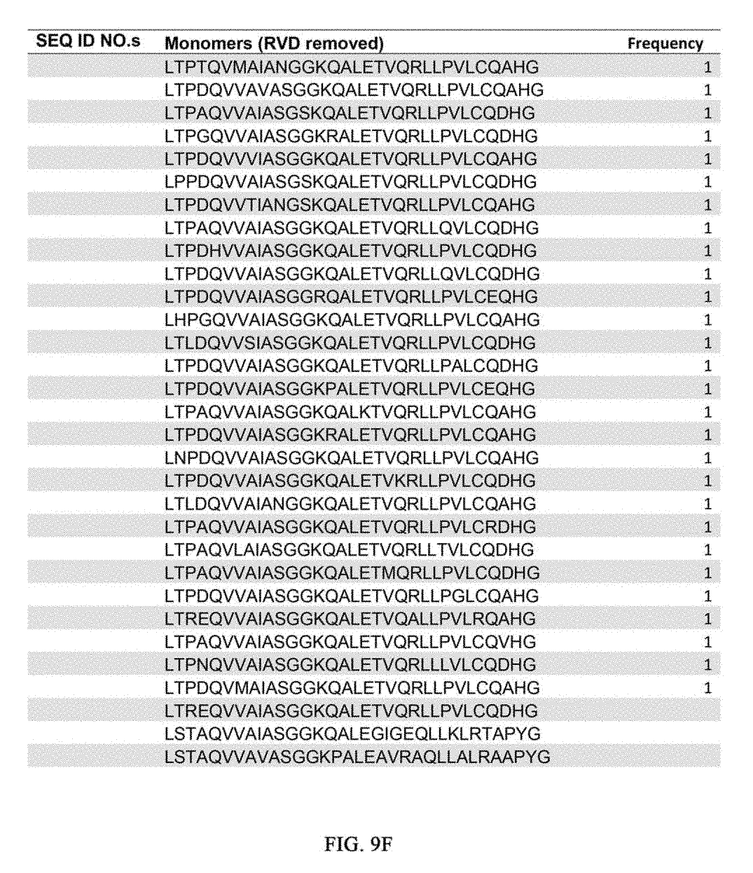

In still further embodiments of the invention, amino acid combinations of interest in the monomers, are [LTPD] (SEQ ID NO: 7) at X₁₋₄ and [KRALE] (SEQ ID NO: 9) at X₁₆₋₂₀ and [AHG] at X₃₂₋₃₄ or [LTPE] (SEQ ID NO: 10) at X₁₋₄ and [KRALE] (SEQ ID NO: 9) at X₁₆₋₂₀ and [DHG] at X₃₂₋₃₄ when the monomer is 34 amino acids in length. When the monomer is 33 or 35 amino acids long, then the corresponding shift occurs in the positions of the contiguous amino acids [KRALE] (SEQ ID NO: 9), [AHG] and [DHG]. In preferred embodiments, the positions of the contiguous amino acids may be ([LTPD] (SEQ ID NO: 7) at X₁₋₄ and [KRALE] (SEQ ID NO: 9) at X₁₅₋₁₉ and [AHG] at X₃₁₋₃₃) or ([LTPE] (SEQ ID NO: 10) at X₁₋₄ and [KRALE] (SEQ ID NO: 9) at X₁₅₋₁₉ and [DHG] at X₃₁₋₃₃) or ([LTPD] (SEQ ID NO: 7) at X₁₋₄ and [KRALE] (SEQ ID NO: 9) at X₁₇₋₂₁ and [AHG] at X₃₃₋₃₅) or ([LTPE] (SEQ ID NO: 10) at X₁₋₄ and [KRALE](SEQ ID NO: 9) at X₁₇₋₂₁ and [DHG] at X₃₃₋₃₅). In still further embodiments of the invention, contiguous amino acids [NGKQALE] (SEQ ID NO: 11) are present at positions X₁₄₋₂₀ or X₁₃₋₁₉ or X₁₅₋₂₁. These representative positions put forward various embodiments of the invention and provide guidance to identify additional amino acids of interest or combinations of amino acids of interest in all the TALE monomers described herein (FIGS. 9A-F and 10).

Provided below are exemplary amino acid sequences of conserved portions of polypeptide monomers (SEQ ID NOS 12-24, respectively, in order of appearance). The position of the RVD in each sequence is represented by XX or by X* (wherein (*) indicates that the RVD is a single amino acid and residue 13 (X₁₃) is absent).

L T P A Q V V A I A S X X G G K Q A L E T V Q R L
L P V L C Q D H G L T P A Q V V A I A S X * G G K
Q A L E T V Q R L L P V L C Q D H G L T P D Q V V
A I A N X X G G K Q A L A T V Q R L L P V L C Q D
H G L T P D Q V V A I A N X X G G K Q A L E T L Q
R L L P V L C Q D H G L T P D Q V V A I A N X X G
G K Q A L E T V Q R L L P V L C Q D H G L T P D Q
V V A I A S X X G G K Q A L A T V Q R L L P V L C
Q D H G L T P D Q V V A I A S X X G G K Q A L E T
V Q R L L P V L C Q D H G L T P D Q V V A I A S X
X G G K Q A L E T V Q R V L P V L C Q D H G L T P
E Q V V A I A S X X G G K Q A L E T V Q R L L P V
L C Q A H G L T P Y Q V V A I A S X X G S K Q A L
E T V Q R L L P V L C Q D H G L T R E Q V V A I A
S X X G G K Q A L E T V Q R L L P V L C Q D H G L
S T A Q V V A I A S X X G G K Q A L E G I G E Q L
L K L R T A P Y G L S T A Q V V A V A S X X G G K
P A L E A V R A Q L L A L R A A P Y G

A further listing of TALE monomers excluding the RVDs which may be denoted in a sequence (X₁₋₁₁-X₁₄₋₃₄ or X₁₋₁₁-X₁₄₋₃₅), wherein X is any amino acid and the subscript is the amino acid position is provided in FIG. 9A-F. The frequency with which each monomer occurs is also indicated.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 25)

M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 26)

R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% dentical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds. There terms “effector domain” and “functional domain” are used interchangeably throughout this application.

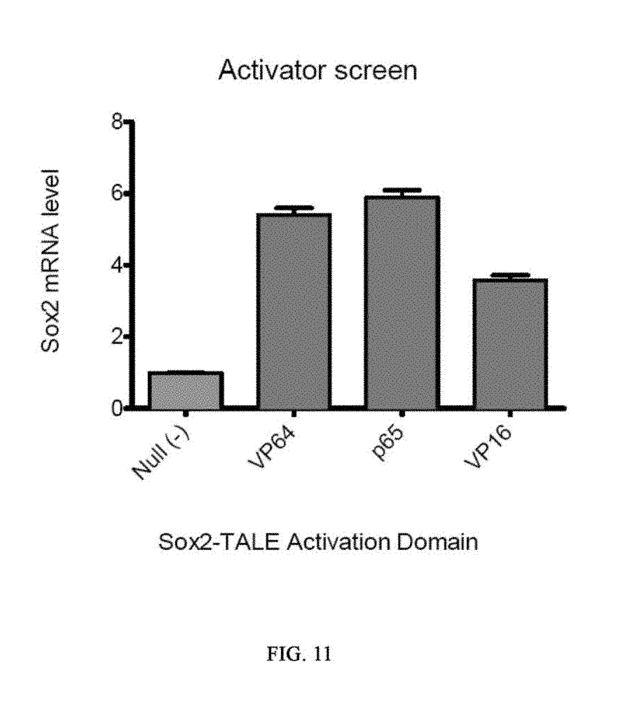

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. A graphical comparison of the effect these different activation domains have on Sox2 mRNA level is provided in FIG. 11.

As used herein, VP16 is a herpesvirus protein. It is a very strong transcriptional activator that specifically activates viral immediate early gene expression. The VP16 activation domain is rich in acidic residues and has been regarded as a classic acidic activation domain (AAD). As used herein, VP64 activation domain is a tetrameric repeat of VP16's minimal activation domain. As used herein, p65 is one of two proteins that the NF-kappa B transcription factor complex is composed of. The other protein is p50. The p65 activation domain is a part of the p65 subunit is a potent transcriptional activator even in the absence of p50. In certain embodiments, the effector domain is a mammalian protein or biologically active fragment thereof. Such effector domains are referred to as “mammalian effector domains.”

In some embodiments, the nucleic acid binding is linked, for example, with an effector domain or functional domain that includes but is not limited to transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

As described in Zhang et al., Nature Biotechnology 29:149-153 (2011), a TALE polypeptide having a nucleic acid binding domain and an effector domain may be used to target the effector domain's activity to a genomic position having a predetermined nucleic acid sequence recognized by the nucleic acid binding domain. In some embodiments of the invention described herein, TALE polypeptides are designed and used for targeting gene regulatory activity, such as transcriptional or translational modifier activity, to a regulatory, coding, and/or intergenic region, such as enhancer and/or repressor activity, that may affect transcription upstream and downstream of coding regions, and may be used to enhance or repress gene expression. For example, TALEs polypeptide may comprise effector domains having DNA-binding domains from transcription factors, effector domains from transcription factors (activators, repressors, co-activators, co-repressors), silencers, nuclear hormone receptors, and/or chromatin associated proteins and their modifiers (e.g., methylases, kinases, phosphatases, acetylases and deacetylases). In a preferred embodiment, the TALE polypeptide may comprise a nuclease domain. In a more preferred embodiment the nuclease domain is a non-specific FokI endonucleases catalytic domain.

In a further embodiment, useful domains for regulating gene expression may also be obtained from the gene products of oncogenes. In yet further advantageous embodiments of the invention, effector domains having integrase or transposase activity may be used to promote integration of exogenous nucleic acid sequence into specific nucleic acid sequence regions, eliminate (knock-out) specific endogenous nucleic acid sequence, and/or modify epigenetic signals and consequent gene regulation, such as by promoting DNA methyltransferase, DNA demethylase, histone acetylase and histone deacetylase activity. In other embodiments, effector domains having nuclease activity may be used to alter genome structure by nicking or digesting target sequences to which the polypeptides of the invention specifically bind, and may allow introduction of exogenous genes at those sites. In still further embodiments, effector domains having invertase activity may be used to alter genome structure by swapping the orientation of a DNA fragment.

In particularly advantageous embodiments, the polypeptides used in the methods of the invention may be used to target transcriptional activity. As used herein, the term “transcription factor” refers to a protein or polypeptide that binds specific DNA sequences associated with a genomic locus or gene of interest to control transcription. Transcription factors may promote (as an activator) or block (as a repressor) the recruitment of RNA polymerase to a gene of interest. Transcription factors may perform their function alone or as a part of a larger protein complex. Mechanisms of gene regulation used by transcription factors include but are not limited to a) stabilization or destabilization of RNA polymerase binding, b) acetylation or deacetylation of histone proteins and c) recruitment of co-activator or co-repressor proteins. Furthermore, transcription factors play roles in biological activities that include but are not limited to basal transcription, enhancement of transcription, development, response to intercellular signaling, response to environmental cues, cell-cycle control and pathogenesis. With regards to information on transcriptional factors, mention is made of Latchman and DS (1997) Int. J. Biochem. Cell Biol. 29 (12): 1305-12; Lee T I, Young R A (2000) Annu. Rev. Genet. 34: 77-137 and Mitchell P J, Tjian R (1989) Science 245 (4916): 371-8, herein incorporated by reference in their entirety.

Light responsiveness of a LITE is achieved via the activation and binding of cryptochrome-2 and CIB1. As mentioned above, blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a LITE system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a LITE stimulated region, allowing for greater precision than vector delivery alone may offer.

The modularity of the LITE system allows for any number of effector domains to be employed for transcriptional modulation. Thus, activator and repressor domains may be selected on the basis of species, strength, mechanism, duration, size, or any number of other parameters.

Applicants next present two prototypical manifestations of the LITE system. The first example is a LITE designed to activate transcription of the mouse gene NEUROG2. The sequence TGAATGATGATAATACGA (SEQ ID NO: 27), located in the upstream promoter region of mouse NEUROG2, was selected as the target and a TALE was designed and synthesized to match this sequence. The TALE sequence was linked to the sequence for cryptochrome-2 via a nuclear localization signal (amino acids: SPKKKRKVEAS (SEQ ID NO: 28)) to facilitate transport of the protein from the cytosol to the nuclear space. A second vector was synthesized comprising the CIB1 domain linked to the transcriptional activator domain VP64 using the same nuclear localization signal. This second vector, also a GFP sequence, is separated from the CIB1-VP64 fusion sequence by a 2A translational skip signal. Expression of each construct was driven by a ubiquitous, constitutive promoter (CMV or EF1-α). Mouse neuroblastoma cells from the Neuro 2A cell line were co-transfected with the two vectors. After incubation to allow for vector expression, samples were stimulated by periodic pulsed blue light from an array of 488 nm LEDs. Unstimulated co-transfected samples and samples transfected only with the fluorescent reporter YFP were used as controls. At the end of each experiment, mRNA was purified from the samples analyzed via qPCR.

Truncated versions of cryptochrome-2 and CIB1 were cloned and tested in combination with the full-length versions of cryptochrome-2 and CIB1 in order to determine the effectiveness of each heterodimer pair. The combination of the CRY2PHR domain, consisting of the conserved photoresponsive region of the cryptochrome-2 protein, and the full-length version of CIB1 resulted in the highest upregulation of Neurog2 mRNA levels (˜22 fold over YFP samples and ˜7 fold over unstimulated co-transfected samples). The combination of full-length cryptochrome-2 (CRY2) with full-length CIB1 resulted in a lower absolute activation level (˜4.6 fold over YFP), but also a lower baseline activation (˜1.6 fold over YFP for unstimulated co-transfected samples). These cryptochrome protein pairings may be selected for particular uses depending on absolute level of induction required and the necessity to minimize baseline “leakiness” of the LITE system.

Speed of activation and reversibility are critical design parameters for the LITE system. To characterize the kinetics of the LITE system, constructs consisting of the Neurog2 TALE-CRY2PHR and CIB1-VP64 version of the system were tested to determine its activation and inactivation speed. Samples were stimulated for as little as 0.5 h to as long as 24 h before extraction. Upregulation of Neurog2 expression was observed at the shortest, 0.5 h, time point (˜5 fold vs YFP samples). Neurog2 expression peaked at 12 h of stimulation (˜19 fold vs YFP samples). Inactivation kinetics were analyzed by stimulating co-transfected samples for 6 h, at which time stimulation was stopped, and samples were kept in culture for 0 to 12 h to allow for mRNA degradation. Neurog2 mRNA levels peaked at 0.5 h after the end of stimulation (˜16 fold vs. YFP samples), after which the levels degraded with an ˜3 h half-life before returning to near baseline levels by 12 h.

The second prototypical example is a LITE designed to activate transcription of the human gene KLF4. The sequence TTCTTACTTATAAC (SEQ ID NO: 29), located in the upstream promoter region of human KLF4, was selected as the target and a TALE was designed and synthesized to match this sequence. The TALE sequence was linked to the sequence for CRY2PHR via a nuclear localization signal (amino acids: SPKKKRKVEAS (SEQ ID NO: 28)). The identical CIB1-VP64 activator protein described above was also used in this manifestation of the LITE system. Human embryonal kidney cells from the HEK293FT cell line were co-transfected with the two vectors. After incubation to allow for vector expression, samples were stimulated by periodic pulsed blue light from an array of 488 nm LEDs. Unstimulated co-transfected samples and samples transfected only with the fluorescent reporter YFP were used as controls. At the end of each experiment, mRNA was purified from the samples analyzed via qPCR.

The light-intensity response of the LITE system was tested by stimulating samples with increased light power (0-9 mW/cm²). Upregulation of KLF4 mRNA levels was observed for stimulation as low as 0.2 mW/cm². KLF4 upregulation became saturated at 5 mW/cm² (2.3 fold vs. YFP samples). Cell viability tests were also performed for powers up to 9 mW/cm² and showed >98% cell viability. Similarly, the KLF4 LITE response to varying duty cycles of stimulation was tested (1.6-100%). No difference in KLF4 activation was observed between different duty cycles indicating that a stimulation paradigm of as low as 0.25 sec every 15 sec should result in maximal activation.

The invention contemplates energy sources such as electromagnetic radiation, sound energy or thermal energy. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The invention particularly relates to inducible methods of perturbing a genomic or epigenomic locus or altering expression of a genomic locus of interest in a cell wherein the genomic or epigenomic locus may be contacted with a non-naturally occurring or engineered composition comprising a deoxyribonucleic acid (DNA) binding polypeptide.

The cells of the present invention may be a prokaryotic cell or a eukaryotic cell, advantageously an animal cell, more advantageously a mammalian cell.

This polypeptide may include a DNA binding domain comprising at least five or more Transcription activator-like effector (TALE) monomers and at least one or more half-monomers specifically ordered to target the genomic locus of interest or at least one or more effector domains linked to a chemical sensitive protein or fragment thereof. The chemical or energy sensitive protein or fragment thereof may undergo a conformational change upon induction by the binding of a chemical source allowing it to bind an interacting partner. The polypeptide may also include a DNA binding domain comprising at least one or more variant TALE monomers or half-monomers specifically ordered to target the genomic locus of interest or at least one or more effector domains linked to the interacting partner, wherein the chemical or energy sensitive protein or fragment thereof may bind to the interacting partner upon induction by the chemical source. The method may also include applying the chemical source and determining that the expression of the genomic locus is altered.

There are several different designs of this chemical inducible system: 1. ABI-PYL based system inducible by Abscisic Acid (ABA), 2. FKBP-FRB based system inducible by rapamycin (or related chemicals based on rapamycin), 3. GID1-GAI based system inducible by Gibberellin (GA).

Another system contemplated by the present invention is a chemical inducible system based on change in sub-cellular localization. Applicants also developed a system in which the polypeptide include a DNA binding domain comprising at least five or more Transcription activator-like effector (TALE) monomers and at least one or more half-monomers specifically ordered to target the genomic locus of interest linked to at least one or more effector domains are further linker to a chemical or energy sensitive protein. This protein will lead to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

This type of system could also be used to induce the cleavage of a genomic locus of interest in a cell when the effector domain is a nuclease.

The designs for this chemical inducible system is an estrogen receptor (ER) based system inducible by 4-hydroxytamoxifen (4OHT). A mutated ligand-binding domain of the estrogen receptor called ERT2 translocates into the nucleus of cells upon binding of 4-hydroxytamoxifen. Two tandem ERT2 domains were linked together with a flexible peptide linker and then fused to the TALE protein targeting a specific sequence in the mammalian genome and linked to one or more effector domains. This polypeptide will be in the cytoplasm of cells in the absence of 4OHT, which renders the TALE protein linked to the effector domains inactive. In the presence of 4OHT, the binding of 4OHT to the tandem ERT2 domain will induce the transportation of the entire peptide into nucleus of cells, allowing the TALE protein linked to the effector domains become active.

In another embodiment of the estrogen receptor (ER) based system inducible by 4-hydroxytamoxifen (4OHT), the present invention may comprise a nuclear exporting signal (NES). Advantageously, the NES may have the sequence of LDLASLIL (SEQ ID NO: 6). In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave. These TRP family proteins respond to different stimuli, including light and heat. When this protein is activated by light or heat, the ion channel will open and allow the entering of ions such as calcium into the plasma membrane. This inflex of ions will bind to intracellular ion interacting partners linked to a polypeptide include TALE protein and one or more effector domains, and the binding will induce the change of sub-cellular localization of the polypeptide, leading to the entire polypeptide entering the nucleus of cells. Once inside the nucleus, the TALE protein linked to the effector domains will be active and modulating target gene expression in cells.

This type of system could also be used to induce the cleavage of a genomic locus of interest in a cell when the effector domain is a nuclease. The light could be generated with a laser or other forms of energy sources. The heat could be generated by raise of temperature results from an energy source, or from nano-particles that release heat after absorbing energy from an energy source delivered in the form of radio-wave.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect. If necessary, the proteins pairings of the LITE system may be altered and/or modified for maximal effect by another energy source.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm² to about 100 W/cm². Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz' (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm² (FDA recommendation), although energy densities of up to 750 mW/cm² have been used. In physiotherapy, ultrasound is typically used as an energy source in a range up to about 3 to 4 W/cm² (WHO recommendation). In other therapeutic applications, higher intensities of ultrasound may be employed, for example, HIFU at 100 W/cm up to 1 kW/cm² (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm⁻². Even more preferably, the exposure to an ultrasound energy source is at a power density of from about 1 to about 15 Wcm⁻².

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm⁻² or 1.25 Wcm⁻² as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

The rapid transcriptional response and endogenous targeting of LITEs make for an ideal system for the study of transcriptional dynamics. For example, LITEs may be used to study the dynamics of mRNA splice variant production upon induced expression of a target gene. On the other end of the transcription cycle, mRNA degradation studies are often performed in response to a strong extracellular stimulus, causing expression level changes in a plethora of genes. LITEs may be utilized to reversibly induce transcription of an endogenous target, after which point stimulation may be stopped and the degradation kinetics of the unique target may be tracked.

The temporal precision of LITEs may provide the power to time genetic regulation in concert with experimental interventions. For example, targets with suspected involvement in long-term potentiation (LTP) may be modulated in organotypic or dissociated neuronal cultures, but only during stimulus to induce LTP, so as to avoid interfering with the normal development of the cells. Similarly, in cellular models exhibiting disease phenotypes, targets suspected to be involved in the effectiveness of a particular therapy may be modulated only during treatment. Conversely, genetic targets may be modulated only during a pathological stimulus. Any number of experiments in which timing of genetic cues to external experimental stimuli is of relevance may potentially benefit from the utility of LITE modulation.

The in vivo context offers equally rich opportunities for the use of LITEs to control gene expression. As mentioned above, photoinducibility provides the potential for previously unachievable spatial precision. Taking advantage of the development of optrode technology, a stimulating fiber optic lead may be placed in a precise brain region. Stimulation region size may then be tuned by light intensity. This may be done in conjunction with the delivery of LITEs via viral vectors or the molecular sleds of U.S. Provisional Patent application No. 61/671,615, or, if transgenic LITE animals were to be made available, may eliminate the use of viruses while still allowing for the modulation of gene expression in precise brain regions. LITEs may be used in a transparent organism, such as an immobilized zebrafish, to allow for extremely precise laser induced local gene expression changes.

The present invention also contemplates a multiplex genome engineering using CRISPR/Cas systems. Functional elucidation of causal genetic variants and elements requires precise genome editing technologies. The type II prokaryotic CRISPR (clustered regularly interspaced short palindromic repeats) adaptive immune system has been shown to facilitate RNA-guided site-specific DNA cleavage. Applicants engineered two different type II CRISPR systems and demonstrate that Cas9 nucleases can be directed by short RNAs to induce precise cleavage at endogenous genomic loci in human and mouse cells. Cas9 can also be converted into a nicking enzyme to facilitate homology-directed repair with minimal mutagenic activity. Finally, multiple guide sequences can be encoded into a single CRISPR array to enable simultaneous editing of several sites within the mammalian genome, demonstrating easy programmability and wide applicability of the CRISPR technology.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, all or a portion of the tracr sequence may also form part of a CRISPR complex, such as by hybridization to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme comprising one or more nuclear localization sequences (NLSs), such as about or more than about 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 30); the NLS from nucleoplasmin (e.g. the nucleoplasmin bipartite NLS with the sequence KRPAATKKAGQAKKKK (SEQ ID NO: 31)); the c-myc NLS having the amino acid sequence PAAKRVKLD (SEQ ID NO: 32) or RQRRNELKRSP (SEQ ID NO: 33); the hRNPA1 M9 NLS having the sequence NQSSNFGPMKGGNFGGRSSGPYGGGGQYFAKPRNQGGY (SEQ ID NO: 34); the sequence RMRIZFKNKGKDTAELRRRRVEVSVELRKAKKDEQILKRRNV (SEQ ID NO: 35) of the IBB domain from importin-alpha; the sequences VSRKRPRP (SEQ ID NO: 36) and PPKKARED (SEQ ID NO: 37) of the myoma T protein; the sequence QPKKKP (SEQ ID NO: 38) of human p53; the sequence SALIKKKKKMAP (SEQ ID NO: 39) of mouse c-abl IV; the sequences DRLRR (SEQ ID NO: 40) and PKQKKRK (SEQ ID NO: 41) of the influenza virus NS1; the sequence RKLKKKIKKL (SEQ ID NO: 42) of the Hepatitis virus delta antigen; the sequence REKKKFLKRR (SEQ ID NO: 43) of the mouse Mxl protein; the sequence KRKGDEVDGVDEVAKKKSKK (SEQ ID NO: 44) of the human poly(ADP-ribose) polymerase; and the sequence RKCLQAGMNLEARKTKK (SEQ ID NO: 45) of the steroid hormone receptors (human) glucocorticoid.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In another embodiment of the present invention, the invention relates to an inducible CRISPR which may comprise an inducible Cas9.

The CRISPR system may be encoded within a vector system which may comprise one or more vectors which may comprise I. a first regulatory element operably linked to a CRISPR/Cas system chimeric RNA (chiRNA) polynucleotide sequence, wherein the polynucleotide sequence may comprise (a) a guide sequence capable of hybridizing to a target sequence in a eukaryotic cell, (b) a tracr mate sequence, and (c) a tracr sequence, and II. a second regulatory element operably linked to an enzyme-coding sequence encoding a CRISPR enzyme which may comprise at least one or more nuclear localization sequences, wherein (a), (b) and (c) are arranged in a 5′ to 3′orientation, wherein components I and II are located on the same or different vectors of the system, wherein when transcribed, the tracr mate sequence hybridizes to the tracr sequence and the guide sequence directs sequence-specific binding of a CRISPR complex to the target sequence, and wherein the CRISPR complex may comprise the CRISPR enzyme complexed with (1) the guide sequence that is hybridized to the target sequence, and (2) the tracr mate sequence that is hybridized to the tracr sequence, wherein the enzyme coding sequence encoding the CRISPR enzyme further encodes a heterologous functional domain.

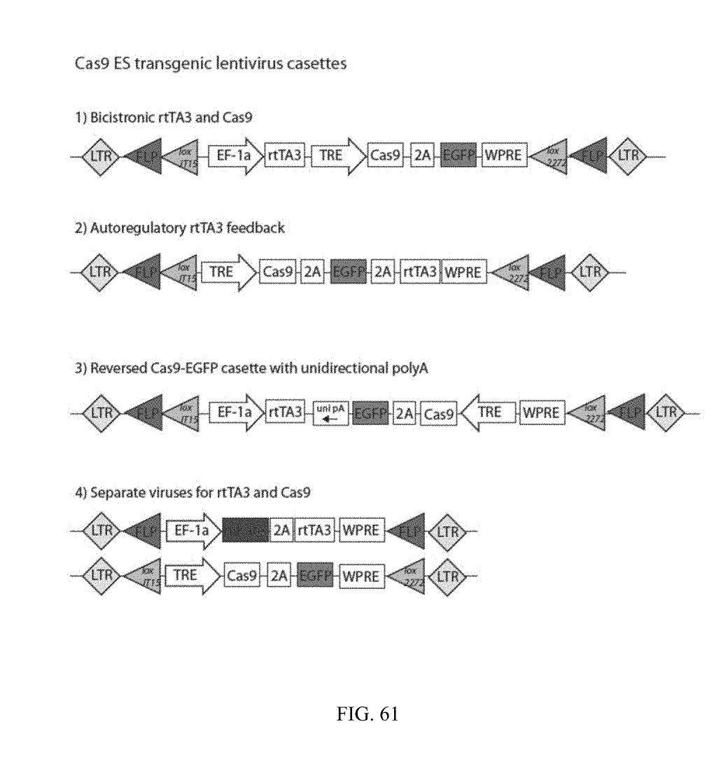

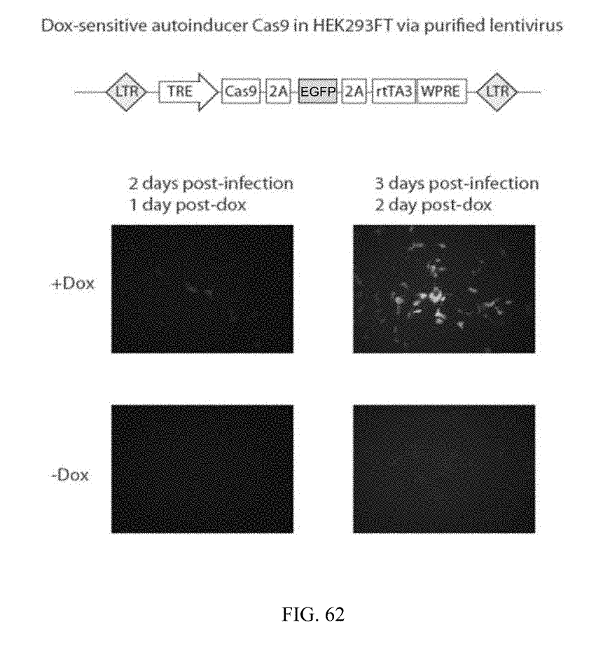

In an advantageous embodiment, the inducible Cas9 may be prepared in a lentivirus. For example, FIG. 61 depicts Tet Cas9 vector designs and FIG. 62 depicts a vector and EGFP expression in 293FT cells. In particular, an inducible tetracycline system is contemplated for an inducible CRISPR. The vector may be designed as described in Markusic et al., Nucleic Acids Research, 2005, Vol. 33, No. 6 e63. The tetracycline-dependent transcriptional regulatory system is based on the Escherichia coli Tn10 Tetracycline resistance operator consisting of the tetracycline repressor protein (TetR) and a specific DNA-binding site, the tetracycline operator sequence (TetO). In the absence of tetracycline, TetR dimerizes and binds to the TetO. Tetracycline or doxycycline (a tetracycline derivative) can bind and induce a conformational change in the TetR leading to its disassociation from the TetO. In an advantageous embodiment, the vector may be a single Tet-On lentiviral vector with autoregulated rtTA expression for regulated expression of the CRISPR complex. Tetracycline or doxycycline may be contemplated for activating the inducible CRISPR complex.

In another embodiment, a cumate gene-switch system is contemplated for an inducible CRISPR. A similar system as described in Mullick et al., BMC Biotechnology 2006, 6:43 doi:10.1186/1472-6750-6-43. The inducible cumate system involves regulatory mechanisms of bacterial operons (cmt and cym) to regulate gene expression in mammalian cells using three different strategies. In the repressor configuration, regulation is mediated by the binding of the repressor (CymR) to the operator site (CuO), placed downstream of a strong constitutive promoter. Addition of cumate, a small molecule, relieves the repression. In the transactivator configuration, a chimaeric transactivator (cTA) protein, formed by the fusion of CymR with the activation domain of VP16, is able to activate transcription when bound to multiple copies of CuO, placed upstream of the CMV minimal promoter. Cumate addition abrogates DNA binding and therefore transactivation by cTA. The invention also contemplates a reverse cumate activator (rcTA), which activates transcription in the presence rather than the absence of cumate. CymR may be used as a repressor that reversibly blocks expression from a strong promoter, such as CMV. Certain aspects of the Cumate repressor/operator system are further described in U.S. Pat. No. 7,745,592.

There exists a pressing need for alternative and robust systems and techniques for sequence targeting with a wide array of applications. This invention addresses this need and provides related advantages. In one aspect, the invention provides a vector system comprising one or more vectors. In some embodiments, the system comprises: (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and (b) a second regulatory element operably linked to an enzyme-coding sequence encoding said CRISPR enzyme comprising a nuclear localization sequence; wherein components (a) and (b) are located on the same or different vectors of the system. In some embodiments, component (a) further comprises the tracr sequence downstream of the tracr mate sequence under the control of the first regulatory element. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the system comprises the tracr sequence under the control of a third regulatory element, such as a polymerase III promoter. In some embodiments, the tracr sequence exhibits at least 50% of sequence complementarity along the length of the tracr mate sequence when optimally aligned. In some embodiments, the CRISPR enzyme comprises one or more nuclear localization sequences of sufficient strength to drive accumulation of said CRISPR enzyme in a detectable amount in the nucleus of a eukaryotic cell. In some embodiments, the CRISPR enzyme is a type II CRISPR system enzyme. In some embodiments, the CRISPR enzyme is a Cas9 enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter. In some embodiments, the guide sequence is at least 15 nucleotides in length. In some embodiments, fewer than 50% of the nucleotides of the guide sequence participate in self-complementary base-pairing when optimally folded.

In one aspect, the invention provides a vector comprising a regulatory element operably linked to an enzyme-coding sequence encoding a CRISPR enzyme comprising one or more nuclear localization sequences. In some embodiments, said regulatory element drives transcription of the CRISPR enzyme in a eukaryotic cell such that said CRISPR enzyme accumulates in a detectable amount in the nucleus of the eukaryotic cell. In some embodiments, the regulatory element is a polymerase II promoter. In some embodiments, the CRISPR enzyme is a type II CRISPR system enzyme. In some embodiments, the CRISPR enzyme is a Cas9 enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRlSPR enzyme lacks DNA strand cleavage activity.

In one aspect, the invention provides a CRISPR enzyme comprising one or more nuclear localization sequences of sufficient strength to drive accumulation of said CRISPR enzyme in a detectable amount in the nucleus of a eukaryotic cell. In some embodiments, the CRISPR enzyme is a type II CRISPR system enzyme. In some embodiments, the CRISPR enzyme is a Cas9 enzyme. In some embodiments, the CRISPR enzyme lacks the ability to cleave one or more strands of a target sequence to which it binds.

In one aspect, the invention provides a eukaryotic host cell comprising (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) a second regulatory element operably linked to an enzyme-coding sequence encoding said CRISPR enzyme comprising a nuclear localization sequence. In some embodiments, the host cell comprises components (a) and (b). In some embodiments, component (a), component (b), or components (a) and (b) are stably integrated into a genome of the host eukaryotic cell. In some embodiments, component (a) further comprises the tracr sequence downstream of the tracr mate sequence under the control of the first regulatory element. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the eukaryotic host cell further comprises a third regulatory element, such as a polymerase III promoter, operably linked to said tracr sequence. In some embodiments, the tracr sequence exhibits at least 50%, 60%, 70%, 80%, 90%, 95%, or 99% of sequence complementarity along the length of the tracr mate sequence when optimally aligned. In some embodiments, the CRISPR enzyme comprises one or more nuclear localization sequences of sufficient strength to drive accumulation of said CRISPR enzyme in a detectable amount in the nucleus of a eukaryotic cell. In some embodiments, the CRISPR enzyme is a type II CRISPR system enzyme. In some embodiments, the CRISPR enzyme is a Cas9 enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length. In some embodiments, fewer than 50%, 40%, 30%, 20%, 10%, or 5% of the nucleotides of the guide sequence participate in self-complementary base-pairing when optimally folded. In one aspect, the invention provides a non-human animal comprising a eukaryotic host cell according to any of the described embodiments.

In one aspect, the invention provides a kit comprising one or more of the components described herein. In some embodiments, the kit comprises a vector system and instructions for using the kit. In some embodiments, the vector system comprises (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) a second regulatory element operably linked to an enzyme-coding sequence encoding said CRISPR enzyme comprising a nuclear localization sequence. In some embodiments, the kit comprises components (a) and (b) located on the same or different vectors of the system. In some embodiments, component (a) further comprises the tracr sequence downstream of the tracr mate sequence under the control of the first regulatory clement. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the system further comprises a third regulatory element, such as a polymerase III promoter, operably linked to said tracr sequence. In some embodiments, the tracr sequence exhibits at least 50%, 60%, 70%, 80%, 90%, 95%, or 99% of sequence complementarity along the length of the tracr mate sequence when optimally aligned. In some embodiments, the CRISPR enzyme comprises one or more nuclear localization sequences of sufficient strength to drive accumulation of said CRISPR enzyme in a detectable amount in the nucleus of a eukaryotic cell. In some embodiments, the CRISPR enzyme is a type II CRISPR system enzyme. In some embodiments, the CRISPR enzyme is a Cas9 enzyme. In some embodiments, the CRISPR enzyme is codon-optimized for expression in a eukmyotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length. In some embodiments, fewer than 50%, 40%, 30%, 20%, 20%, 10% or 5% of the nucleotides of the guide sequence participate in self-complementary base-pairing when optimally folded.

In one aspect, the invention provides a computer system for selecting a candidate target sequence within a nucleic acid sequence in a eukaryotic cell for targeting by a CRISPR complex. In some embodiments, the computer system comprises (a) a memory unit configured to receive and/or store said nucleic acid sequence; and (b) one or more processors alone or in combination programmed to (i) locate a CRISPR motif sequence within said nucleic acid sequence, and (ii) select a sequence adjacent to said located CRlSPR motif sequence as the candidate target sequence to which the CRISPR complex binds. In some embodiments, said locating step comprises identifying a CRISPR motif sequence located less than about 10000 nucleotides away from said target sequence, such as less than about 5000, 2500, 1000, 500, 250, 100, 50, 25, or fewer nucleotides away from the target sequence. In some embodiments, the candidate target sequence is at least 10, 15, 20, 25, 30, or more nucleotides in length. In some embodiments, the nucleotide at the 3′ end of the candidate target sequence is located no more than about 10 nucleotides upstream of the CRISPR motif sequence, such as no more than 5, 4, 3, 2, or 1 nucleotides. In some embodiments, the nucleic acid sequence in the eukaryotic cell is endogenous to the eukaryotic genome. In some embodiments, the nucleic acid sequence in the eukaryotic cell is exogenous to the eukaryotic genome.

In one aspect, the invention provides a computer-readable medium comprising codes that, upon execution by one or more processors, implements a method of selecting a candidate target sequence within a nucleic acid sequence in a eukaryotic cell for targeting by a CRISPR complex, said method comprising: (a) locating a CRISPR motif sequence within said nucleic acid sequence, and (b) selecting a sequence adjacent to said located CRISPR motif sequence as the candidate target sequence to which the CRISPR complex binds. In some embodiments, said locating comprises locating a CRISPR motif sequence that is less than about 5000, 2500, 1000, 500, 250, 100, 50, 25, or fewer nucleotides away from said target sequence. In some embodiments, the candidate target sequence is at least 10, 15, 20, 25, 30, or more nucleotides in length. In some embodiments, the nucleotide at the 3′ end of the candidate target sequence is located no more than about 10 nucleotides upstream of the CRISPR motif sequence, such as no more than 5, 4, 3, 2, or 1 nucleotides. In some embodiments, the nucleic acid sequence in the eukaryotic cell is endogenous to the eukaryotic genome. In some embodiments, the nucleic acid sequence in the eukaryotic cell is exogenous to the eukaryotic genome.

In one aspect, the invention provides a method of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. In some embodiments, said cleavage comprises cleaving one or two strands at the location of the target sequence by said CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein the one or more vectors drive expression of one or more of: the CRISPR enzyme, the guide sequence linked to the tracr mate sequence, and the tracr sequence. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of a polynucleotide in a eukaryotic cell. In some embodiments, the method comprises allowing a CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence. In some embodiments, the method further comprises delivering one or more vectors to said eukaryotic cells, wherein the one or more vectors drive expression of one or more of: the CRISPR enzyme, the guide sequence linked to the tracr mate sequence, and the tracr sequence.

In one aspect, the invention provides a method of generating a model eukaryotic cell comprising a mutated disease gene. In some embodiments, a disease gene is any gene associated an increase in the risk of having or developing a disease. In some embodiments, the method comprises (a) introducing one or more vectors into a eukaryotic cell, wherein the one or more vectors drive expression of one or more of: a CRISPR enzyme, a guide sequence linked to a tracr mate sequence, and a tracr sequence; and (b) allowing a CRISPR complex to bind to a target polynucleotide to effect cleavage of the target polynucleotide within said disease gene, wherein the CRISPR complex comprises the CRISPR enzyme complexed with (1) the guide sequence that is hybridized to the target sequence within the target polynucleotide, and (2) the tracr mate sequence that is hybridized to the tracr sequence, thereby generating a model eukaryotic cell comprising a mutated disease gene. In some embodiments, said cleavage comprises cleaving one or two strands at the location of the target sequence by said CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

In one aspect, the invention provides a method for developing a biologically active agent that modulates a cell signaling event associated with a disease gene. In some embodiments, a disease gene is any gene associated an increase in the risk of having or developing a disease. In some embodiments, the method comprises (a) contacting a test compound with a model cell of any one of the described embodiments; and (b) detecting a change in a readout that is indicative of a reduction or an augmentation of a cell signaling event associated with said mutation in said disease gene, thereby developing said biologically active agent that modulates said cell signaling event associated with said disease gene.

In one aspect, the invention provides a recombinant polynucleotide comprising a guide sequence upstream of a tracr mate sequence, wherein the guide sequence when expressed directs sequence-specific binding of a CRISPR complex to a corresponding target sequence present in a eukaryotic cell. In some embodiments, the target sequence is a viral sequence present in a eukaryotic cell. In some embodiments, the target sequence is a proto-oncogene or an oncogene.

In one aspect, the invention provides a vector system comprising one or more vectors. In some embodiments, the vector system comprises (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and (b) a second regulatory element operably linked to an enzyme-coding sequence encoding said CRISPR enzyme comprising a nuclear localization sequence; wherein components (a) and (b) are located on the same or different vectors of the system.

In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors arc capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSecl (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a pmiicular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43:235-275), in particular promoters off cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264, 166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoc et al., Emerg. Infect. Dis., 5:254-263 [1999]; Mascpohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacterial., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, all or a portion of the tracr sequence may also form part of a CRISPR complex, such as by hybridization to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr-6, Csb1, Csb2, Csb3, Csxl7, Csxl4, Csxl0, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. Sec Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme comprising one or more nuclear localization sequences (NLSs), such as about or more than about 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 30); the NLS from nucleoplasmin (e.g. the nucleoplasmin bipartite NLS with the sequence KRPAATKKAGQAKKKK (SEQ ID NO: 31)); the c-myc NLS having the amino acid sequence PAAKRVKLD (SEQ ID NO: 32) or RQRRNELKRSP (SEQ ID NO: 33); the hRNPA1 M9 NLS having the sequence NQSSNFGPMKGGNFGGRSSGPYGGGGQYFAKPRNQGGY (SEQ ID NO: 34); the sequence RMRIZFKNKGKDTAELRRRRVEVSVELRKAKKDEQILKRRNV (SEQ ID NO: 35) of the IBB domain from importin-alpha; the sequences VSRKRPRP (SEQ ID NO: 36) and PPKKARED (SEQ ID NO: 37) of the myoma T protein; the sequence PQPKKKP (SEQ ID NO: 38) of human p53; the sequence SALlKKKKKMAP (SEQ ID NO: 39) of mouse c-ablIV; the sequences DRLRR (SEQ ID NO: 40) and PKQKKRK (SEQ ID NO: 41) of the influenza virus NS1; the sequence RKLKKKIKKL (SEQ ID NO: 42) of the Hepatitis virus delta antigen; the sequence REKKKFLKRR (SEQ ID NO: 43) of the mouse Mxl protein; the sequence KRKGDEVDGVDEVAKKKSKK (SEQ ID NO: 44) of the human poly(ADP-ribose) polymerase; and the sequence RKCLQAGMNLEARKTKK (SEQ ID NO: 45) of the steroid hormone receptors (human) glucocorticoid.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 514) where NNNNNNNNNNNNXGG (SEQ ID NO: 515) (N is A, G, T, or C; and X can be anything) has a single occurrence in the genome. A unique target sequence in a genome may include an S. pyogenes Cas9 target site of the form MMMMMMMMMNNNNNNNNNNNXGG (SEQ ID NO: 516) where NNNNNNNNNNNXGG (SEQ ID NO: 517) (N is A, G, T, or C; and X can be anything) has a single occurrence in the genome. For the S. thermophilus CRISPR1 Cas9, a unique target sequence in a genome may include a Cas9 target site of the form MMMMMMMMNNNNNNNNNNNNXXAGAAW (SEQ ID NO: 518) where NNNNNNNNNNNNXXAGAAW (SEQ ID NO: 519) (N is A, G, T, or C; X can be anything; and W is A or T) has a single occurrence in the genome. A unique target sequence in a genome may include an S. thermophilus CRISPR1 Cas9 target site of the form MMMMMMMMMNNNNNNNNNNNXXAGAAW (SEQ ID NO: 520) where NNNNNNNNNNNXXAGAAW (SEQ ID NO: 521) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNNNNXGGXG (SEQ ID NO: 522) where NNNNNNNNNNNNXGGXG (SEQ ID NO: 523) (N is A, G, T, or C; and X can be anything) has a single occurrence in the genome. A unique target sequence in a genome may include an S. pyogenes Cas9 target site of the form MMMMMMMMMNNNNNNNNNNNXGGXG (SEQ ID NO: 524) where NNNNNNNNNNNXGGXG (SEQ ID NO: 525) (N is A, G, T, or C; and X can be anything) has a single occurrence in the genome. In each of these sequences “M” may be A, G, T, or C, and need not be considered in identifying a sequence as unique.

In some embodiments, a guide sequence is selected to reduce the degree secondary structure within the guide sequence. In some embodiments, about or less than about 75%, 50%, 40%, 30%, 25%, 20%, 15%, 10%, 5%, 1%, or fewer of the nucleotides of the guide sequence participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62).

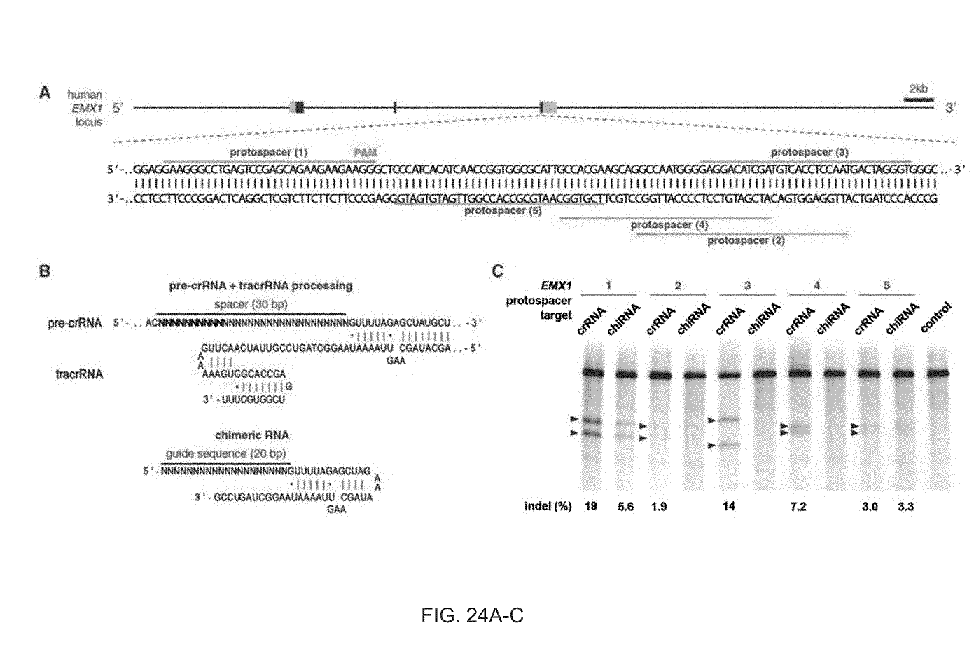

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. Example illustrations of optimal alignment between a tracr sequence and a tracr mate sequence are provided in FIGS. 24B AND 304B. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. An example illustration of such a hairpin structure is provided in the lower portion of FIG. 24B, where the portion of the sequence 5′ of the final “N’ and upstream of the loop corresponds to the tracr mate sequence, and the portion of the sequence 3′ of the loop corresponds to the tracr sequence. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNNNNNNNNNNNNNgtttttgtactctcaagatttaGAAAtaaatcttgcagaagctacaaagataaggctt catgccgaaatc aacaccctgtcattttatggcagggtgttttcgttatttaaTTTTTT (SEQ ID NO: 526); (2) NNNNNNNNNNNNNNNNNNNNgtttttgtactctcaGAAAtgcagaagctacaaagataaggcttcatgccgaaatca acaccctgtcatt ttatggcagggtgttttcgttatttaaTTTTTT (SEQ ID NO: 527); (3) NNNNNNNNNNNNNNNNNNNNgtttttgtactctcaGAAAtgcagaagctacaaagataaggcttcatgccgaaatca acaccctgtcattttatggcagggtgtTTTTTT (SEQ ID NO: 528); (4) NNNNNNNNNNNNNNNNNNNNgttttagagctaGAAAtagcaagttaaaataaggctagtccgttatcaacttgaaaa agtggcaccgagtcggtgcTTTTTT (SEQ ID NO: 529); (5) NNNNNNNNNNNNNNNNNNNNgttttagagctaGAAATAGcaagttaaaataaggctagtccgttatcaacttgaa aaagtgTTTTTTT (SEQ ID NO: 530); and (6) NNNNNNNNNNNNNNNNNNNNgttttagagctagAAATAGcaagttaaaataaggctagtccgttatcaTTTTT TTT (SEQ ID NO: 531). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence (such as illustrated in the top portion of FIG. 24B).

In some embodiments, a recombination template is also provided. A recombination template may be a component of another vector as described herein, contained in a separate vector, or provided as a separate polynucleotide. In some embodiments, a recombination template is designed to serve as a template in homologous recombination, such as within or near a target sequence nicked or cleaved by a CRISPR enzyme as a part of a CRISPR complex. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In some embodiments, a CRISPR enzyme may form a component of a Light Inducible Transcriptional Effector (LITE) to direct changes in transcriptional activity in a sequence-specific manner. The components of a light may include a CRISPR enzyme, a light-responsive cytochrome heterodimer (e.g. from Arabidopsis thaliana), and a transcriptional activation/repression domain. A guide sequence may be selected to direct CRISPR complex formation at a promoter sequence of a gene of interest. The CRISPR enzyme may be fused to one half of the cryptochrome heterodimer (cryptochrome-2 or CIB1), while the remaining cryptochrome partner is fused to a transcriptional effector domain. Effector domains may be either activators, such as VP16, VP64, or p65, or repressors, such as KRAB, EnR, or SID. In a LITE's unstimulated state, the CRISPR-cryptochrome2 protein localizes to the promoter of the gene of interest, but is not bound to the CIB1-effector protein. Upon stimulation of a LITE with blue spectrum light, cryptochrome-2 becomes activated, undergoes a conformational change, and reveals its binding domain. CIB1, in turn, binds to cryptochrome-2 resulting in localization of the effector domain to the promoter region of the gene of interest and initiating gene overexpression or silencing. Activator and repressor domains may selected on the basis of species, strength, mechanism, duration, size, or any number of other parameters. Preferred effector domains include, but are not limited to, a transposase domain, integrase domain, recombinase domain, resolvase domain, invertase domain, protease domain, DNA methyltransferase domain, DNA demethylase domain, histone acetylase domain, histone deacetylases domain, nuclease domain, repressor domain, activator domain, nuclear-localization signal domains, transcription-protein recruiting domain, cellular uptake activity associated domain, nucleic acid binding domain or antibody presentation domain. Further examples of inducible DNA binding proteins and methods for their use are provided in U.S. 61/736,465, which is hereby incorporated by reference in its entirety.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and animals comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Cuttent Topics in Microbiology and Immunology Doerfler and Bohm (eds) (1995); and Yu et al., Gene Therapy 1:13-26 (1994).

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producer a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mlMCD-3, NHDF, HeLa-S3, Huhl, Huh4, Huh7, HUVEC, HASMC, HEKn, HEKa, MiaPaCell, Panel, PC-3, TF1, CTLL-2, ClR, Rat6, CVl, RPTE, Al0, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bel-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10Tl/2, C6/36, Cal-27, CHO, CHO-7, CHO-IR, CHO-Kl, CHO-K2, CHO-T, CHO Dhfr−/−, COR-L23, COR-L23/CPR, COR-L23/5010, COR-L23/R23, COS-7, COV-434, CML Tl, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYOl, LNCap, Ma-Mel1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN 1OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rat, or rabbit. Methods for producing transgenic plants and animals are known in the art, and generally begin with a method of cell transfection, such as described herein.

In one aspect, the invention provides for methods of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

In one aspect, the invention provides a method of modifying expression of a polynucleotide in a eukaryotic cell. In some embodiments, the method comprises allowing a CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence.

In one aspect, the invention provides a computer system for selecting one or more candidate target sequences within a nucleic acid sequence in a eukaryotic cell for targeting by a CRISPR complex. In some embodiments, the system comprises (a) a memory unit configured to receive and/or store said nucleic acid sequence; and (b) one or more processors alone or in combination programmed to (i) locate a CRISPR motif sequence within said nucleic acid sequence, and (ii) select a sequence adjacent to said located CRISPR motif sequence as the candidate target sequence to which the CRlSPR complex binds.

In one aspect, the invention provides a computer readable medium comprising codes that, upon execution by one or more processors, implements a method of selecting a candidate target sequence within a nucleic acid sequence in a eukaryotic cell for targeting by a CRISPR complex. In some embodiments, the method comprises (a) locating a CRISPR motif sequence within said nucleic acid sequence, and (b) selecting a sequence adjacent to said located CRISPR motif sequence as the candidate target sequence to which the CRISPR complex binds.

A computer system (or digital device) may be used to receive and store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system comprises one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device comprising a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

In one aspect, the invention provides kits containing any one or more of the elements disclosed in the above methods and compositions. In some embodiments, the kit comprises a vector system and instructions for using the kit. In some embodiments, the vector system comprises (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) a second regulatory element operably linked to an enzyme-coding sequence encoding said CRISPR enzyme comprising a nuclear localization sequence. Elements may provide individually or in combinations, and may provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types. As such the CRISPR complex of the invention has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide. The guide sequence is linked to a tracr mate sequence, which in turn hybridizes to a tracr sequence.

In one embodiment, this invention provides a method of cleaving a target polynucleotide. The method comprises modifying a target polynucleotide using a CRISPR complex that binds to the target polynucleotide and effect cleavage of said target polynucleotide. Typically, the CRISPR complex of the invention, when introduced into a cell, creates a break (e.g., a single or a double strand break) in the genome sequence. For example, the method can be used to cleave a disease gene in a cell.

The break created by the CRISPR complex can be repaired by a repair process such as a homology-directed repair process. During the repair process, an exogenous polynucleotide template can be introduced into the genome sequence. In some methods, a homology-directed repair process is used modify genome sequence. For example, an exogenous polynucleotide template comprising a sequence to be integrated flanked by an upstream sequence and a downstream sequence is introduced into a cell. The upstream and downstream sequences share sequence similarity with either side of the site of integration in the chromosome.

Where desired, a donor polynucleotide can be DNA, e.g., a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

The upstream and downstream sequences in the exogenous polynucleotide template are selected to promote recombination between the chromosomal sequence of interest and the donor polynucleotide. The upstream sequence is a nucleic acid sequence that shares sequence similarity with the genome sequence upstream of the targeted site for integration. Similarly, the downstream sequence is a nucleic acid sequence that shares sequence similarity with the chromosomal sequence downstream of the targeted site of integration. The upstream and downstream sequences in the exogenous polynucleotide template can have 75%, 80%, 85%, 90%, 95%, or 100% sequence identity with the targeted genome sequence. Preferably, the upstream and downstream sequences in the exogenous polynucleotide template have about 95%, 96%, 97%, 98%, 99%, or 100% sequence identity with the targeted genome sequence. In some methods, the upstream and downstream sequences in the exogenous polynucleotide template have about 99% or 100% sequence identity with the targeted genome sequence.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the invention can be constructed using recombinant techniques (see, for example, Sambrook et al., 2001 and Ausubel et al., 1996).

In an exemplary method for modifying a target polynucleotide by integrating an exogenous polynucleotide template, a double stranded break is introduced into the genome sequence by the CRISPR complex, the break is repaired via homologous recombination an exogenous polynucleotide template such that the template is integrated into the genome. The presence of a double-stranded break facilitates integration of the template.

In other embodiments, this invention provides a method of modifying expression of a polynucleotide in a eukaryotic cell. The method comprises increasing or decreasing expression of a target polynucleotide by using a CRISPR complex that binds to the polynucleotide.

Where desired, to effect the modification of the expression in a cell, one or more vectors comprising a tracr sequence, a guide sequence linked to the tracr mate sequence, a sequence encoding a CRISPR enzyme is delivered to a cell. In some methods, the one or more vectors comprises a regulatory element operably linked to an enzyme-coding sequence encoding said CRISPR enzyme comprising a nuclear localization sequence; and a regulatory element operably linked to a tracr mate sequence and one or more insertion sites for inserting a guide sequence upstream of the tracr mate sequence. When expressed, the guide sequence directs sequence-specific binding of a CRISPR complex to a target sequence in a cell. Typically, the CRISPR complex comprises a CRISPR enzyme complexed with (1) the guide sequence that is hybridized to the target sequence, and (2) the tracr mate sequence that is hybridized to the tracr sequence.

In some methods, a target polynucleotide can be inactivated to effect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

In some methods, a control sequence can be inactivated such that it no longer functions as a control sequence. As used herein, “control sequence” refers to any nucleic acid sequence that effects the transcription, translation, or accessibility of a nucleic acid sequence. Examples of a control sequence include, a promoter, a transcription terminator, and an enhancer are control sequences.

The inactivated target sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). In some methods, the inactivation of a target sequence results in “knock-out” of the target sequence.

A method of the invention may be used to create an animal or cell that may be used as a disease model. As used herein, “disease” refers to a disease, disorder, or indication in a subject. For example, a method of the invention may be used to create an animal or cell that comprises a modification in one or more nucleic acid sequences associated with a disease, or an animal or cell in which the expression of one or more nucleic acid sequences associated with a disease are altered. Such a nucleic acid sequence may encode a disease associated protein sequence or may be a disease associated control sequence.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of one or more of a CRISPR enzyme, a guide sequence linked to a tracr mate sequence, and a tracr sequence; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the CRISPR complex of the invention on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically propmiional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probe (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent:protein complex so formed. In one aspect of this embodiment, the agent that specifically binds a protein associated with a signaling biochemical pathway is an antibody, preferably a monoclonal antibody.

The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure requires the agent to contain a label introduced either chemically or enzymatically. A desirable label generally does not interfere with binding or the stability of the resulting agent:polypeptide complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyro sine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2α). Alternatively, these antibodies can be generated using conventional polyclonal or monoclonal antibody technologies by immunizing a host animal or an antibody-producing cell with a target protein that exhibits the desired post-translational modification.

In practicing the subject method, it may be desirable to discern the expression pattern of an protein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

An altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays such as AlphaScreen™ (available from Perkin Elmer) and eTag™ assay (Chan-Hui, et al. (2003) Clinical Immunology III: 162-174).

Where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are particularly suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a minisecond.

In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Examples of disease-associated genes and polynucleotides are listed in Tables A and B. In Table B, a six-digit number following an entry in the Disease/Disorder/Indication column is an OMIM number (Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), available on the World Wide Web. A number in parentheses after the name of each disorder indicates whether the mutation was positioned by mapping the wildtype gene (1), by mapping the disease phenotype itself (2), or by both approaches (3). For example, a “(3)”, includes mapping of the wildtype gene combined with demonstration of a mutation in that gene in association with the disorder.”

Examples of signaling biochemical pathway-associated genes and polynucleotides are listed in Table C.

TABLE A

| DISEASE/DISORDERS | GENE(S) |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion - related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); Il-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE B

| DISEASE/DISORDER/INDICATION | GENE(S) |
|---|---|
| 17,20-lyase deficiency, isolated, 202110 (3) | CYP17A1, CYP17, P450C17 |
| 17-alpha-hydroxylase/17,20-lyase | CYP17A1, CYP17, P450C17 |
| deficiency, 202110 (3) | |
| 2-methyl-3-hydroxybutyryl-CoA | HADH2, ERAB |
| dehydrogenase deficiency, 300438 (3) | |
| 2-methylbutyrylglycinuria (3) | ACADSB |
| 3-beta-hydroxysteroid dehydrogenase, type | HSD3B2 |
| II, deficiency (3) | |
| 3-hydroxyacyl-CoA dehydrogenase | HADHSC, SCHAD |
| deficiency, 609609 (3) | |
| 3-Methylcrotonyl-CoA carboxylase 1 | MCCC1, MCCA |
| deficiency, 210200 (3) | |
| 3-Methylcrotonyl-CoA carboxylase 2 | MCCC2, MCCB |
| deficiency, 210210 (3) | |
| 3-methylglutaconic aciduria, type I, 250950 | AUH |
| (3) | |
| 3-methylglutaconicaciduria, type III, 258501 | OPA3, MGA3 |
| (3) | |
| 3-M syndrome, 273750 (3) | CUL7 |
| 6-mercaptopurine sensitivity (3) | TPMT |
| Aarskog-Scott syndrome (3) | FGD1, FGDY, AAS |
| Abacavir hypersensitivity, susceptibility to | HLA-B |
| (3) | |
| ABCD syndrome, 600501 (3) | EDNRB, HSCR2, ABCDS |
| Abetalipoproteinemia, 200100 (3) | MTP |
| Abetalipoproteinemia (3) | APOB, FLDB |
| Acampomelic campolelic dysplasia, 114290 | SOX9, CMD1, SRA1 |
| (3) | |
| Acatalasemia (3) | CAT |
| Accelerated tumor formation, susceptibility | MDM2 |
| to (3) | |
| Achalasia-addisonianism-alacrimia | AAAS, AAA |
| syndrome, 231550 (3) | |
| Acheiropody, 200500 (3) | C7orf2, ACHP, LMBR1 |
| Achondrogenesis-hypochondrogenesis, | COL2A1 |
| type II, 200610 (3) | |
| Achondrogenesis Ib, 600972 (3) | SLC26A2, DTD, DTDST, D5S1708, |
| | EDM4 |
| Achondroplasia, 100800 (3) | FGFR3, ACH |
| Achromatopsia-2, 216900 (3) | CNGA3, CNG3, ACHM2 |
| Achromatopsia-3, 262300 (3) | CNGB3, ACHM3 |
| Achromatopsia-4 (3) | GNAT2, ACHM4 |
| Acid-labile subunit, deficiency of (3) | IGFALS, ALS |
| Acquired long QT syndrome, susceptibility | KCNH2, LQT2, HERG |
| to (3) | |
| Acrocallosal syndrome, 200990 (3) | GLI3, PAPA, PAPB, ACLS |
| Acrocapitofemoral dysplasia, 607778 (3) | IHH, BDA1 |
| Acrodermatitis enteropathica, 201100 (3) | SLC39A4, ZIP4 |
| Acrokeratosis verruciformis, 101900 (3) | ATP2A2, ATP2B, DAR |
| Acromegaly, 102200 (3) | GNAS, GNAS1, GPSA, POH, PHP1B, |
| | PHP1A, AHO |
| Acromegaly, 102200 (3) | SSTR5 |
| Acromesomelic dysplasia, Hunter- | GDF5, CDMP1 |
| Thompson type, 201250 (3) | |
| Acromesomelic dysplasia, Maroteaux type, | NPR2, ANPRB, AMDM |
| 602875 (3) | |
| Acyl-CoA dehydrogenase, long chain, | ACADL, LCAD |
| deficiency of (3) | |
| Acyl-CoA dehydrogenase, medium chain, | ACADM, MCAD |
| deficiency of, 201450 (3) | |
| Acyl-CoA dehydrogenase, short-chain, | ACADS, SCAD |
| deficiency of, 201470 (3) | |
| Adenocarcinoma of lung, response to | EGFR |
| tyrosine kinase inhibitor in, 211980 (3) | |
| Adenocarcinoma of lung, somatic, 211980 | BRAF |
| (3) | |
| Adenocarcinoma of lung, somatic, 211980 | ERBB2, NGL, NEU, HER2 |
| (3) | |
| Adenocarcinoma of lung, somatic, 211980 | PRKN, PARK2, PDJ |
| (3) | |
| Adenocarcinoma, ovarian, somatic (3) | PRKN, PARK2, PDJ |
| Adenoma, periampullary (3) | APC, GS, FPC |
| Adenomas, multiple colorectal, 608456 (3) | MUTYH |
| Adenomas, salivary gland pleomorphic, | PLAG1, SGPA, PSA |
| 181030 (3) | |
| Adenomatous polyposis coli (3) | APC, GS, FPC |
| Adenomatous polyposis coli, attenuated (3) | APC, GS, FPC |
| Adenosine deaminase deficiency, partial, | ADA |
| 102700 (3) | |
| Adenylosuccinase deficiency, 103050 (3) | ADSL |
| Adiponectin deficiency (3) | APM1, GBP28 |
| Adrenal adenoma, sporadic (3) | MEN1 |
| Adrenal cortical carcinoma, 202300 (3) | TP53, P53, LFS1 |
| Adrenal hyperplasia, congenital, due to 11- | CYP11B1, P450C11, FHI |
| beta-hydroxylase deficiency (3) | |
| Adrenal hyperplasia, congenital, due to 21- | CYP21A2, CYP21, CA21H |
| hydroxylase deficiency (3) | |
| Adrenal hyperplasia, congenital, due to | POR |
| combined P450C17 and P450C21 | |
| deficiency, 201750 (3) | |
| Adrenal hypoplasia, congenital, with | DAX1, AHC, AHX, NROB1 |
| hypogonadotropic hypogonadism, 300200 | |
| (3) | |
| Adrenocortical insufficiency without ovarian | FTZF1, FTZ1, SF1 |
| defect (3) | |
| Adrenocortical tumor, somatic (3) | PRKAR1A, TSE1, CNC1, CAR |
| Adrenocorticotropic hormone deficiency, | TBS19 |
| 201400 (3) | |
| Adrenoleukodystrophy, 300100 (3) | ABCD1, ALD, AMN |
| Adrenoleukodystrophy, neonatal, 202370 | PEX10, NALD |
| (3) | |
| Adrenoleukodystrophy, neonatal, 202370 | PEX13, ZWS, NALD |
| (3) | |
| Adrenoleukodystrophy, neonatal, 202370 | PEX1, ZWS1 |
| (3) | |
| Adrenoleukodystrophy, neonatal, 202370 | PEX26 |
| (3) | |
| Adrenoleukodystrophy, neonatal, 202370 | PXR1, PEX5, PTS1R |
| (3) | |
| Adrenomyeloneuropathy, 300100 (3) | ABCD1, ALD, AMN |
| Adult i phenotype with congenital cataract, | GCNT2 |
| 110800 (3) | |
| Adult i phenotype without cataract, 110800 | GCNT2 |
| (3) | |
| ADULT syndrome, 103285 (3) | TP73L, TP63, KET, EEC3, SHFM4, |
| | LMS, RHS |
| Advanced sleep phase syndrome, familial, | PER2, FASPS, KIAA0347 |
| 604348 (3) | |
| Afibrinogenemia, 202400 (3) | FGA |
| Afibrinogenemia, congenital, 202400 (3) | FGB |
| Agammaglobulinemia, 601495 (3) | IGHM, MU |
| Agammaglobulinemia, autosomal recessive | IGLL1, IGO, IGL5, VPREB2 |
| (3) | |
| Agammaglobulinemia, non-Bruton type, | LRRC8, KIAA1437 |
| 601495 (3) | |
| Agammaglobulinemia, type 1, X-linked (3) | BTK, AGMX1, IMD1, XLA, AT |
| AGAT deficiency (3) | GATM, AGAT |
| Agenesis of the corpus callosum with | SLC12A6, KCC3A, KCC3B, KCC3, |
| peripheral neuropathy, 218000 (3) | ACCPN |
| AICA-ribosiduria due to ATIC deficiency, | ATIC, PURH, AICAR |
| 608688 (3) | |
| AIDS, delayed/rapid progression to (3) | KIR3DL1, NKAT3, NKB1, AMB11, |
| | KIR3DS1 |
| AIDS, rapid progression to, 609423 (3) | IFNG |
| AIDS, resistance to (3) | CXCL12, SDF1 |
| Alagille syndrome, 118450 (3) | JAG1, AGS, AHD |
| Albinism, brown oculocutaneous, (3) | OCA2, P, PED, D15S12, BOCA |
| Albinism, ocular, autosomal recessive (3) | OCA2, P, PED, D15S12, BOCA |
| Albinism, oculocutaneous, type IA, 203100 | TYR |
| (3) | |
| Albinism, oculocutaneous, type IB, 606952 | TYR |
| (3) | |
| Albinism, oculocutaneous, type II (3) | OCA2, P, PED, D15S12, BOCA |
| Albinism, rufous, 278400 (3) | TYRP1, CAS2, GP75 |
| Alcohol dependence, susceptibility to, | HTR2A |
| 103780 (3) | |
| Alcohol intolerance, acute (3) | ALDH2 |
| Alcoholism, susceptibility to, 103780 (3) | GABRA2 |
| Aldolase A deficiency (3) | ALDOA |
| Aldosterone to renin ratio raised (3) | CYP11B2 |
| Aldosteronism, glucocorticoid-remediable, | CYP11B1, P450C11, FHI |
| 103900 (3) | |
| Alexander disease, 203450 (3) | GFAP |
| Alexander disease, 203450 (3) | NDUFV1, UQOR1 |
| Alkaptonuria, 203500 (3) | HGD, AKU |
| Allan-Herndon-Dudley syndrome, 300523 | SLC16A2, DXS128, XPCT |
| (3) | |
| Allergic rhinitis, susceptibility to, 607154 (3) | IL13, ALRH |
| Alopecia universalis, 203655 (3) | HR, AU |
| Alpers syndrome, 203700 (3) | POLG, POLG1, POLGA, PEO |
| Alpha-1-antichymotrypsin deficiency (3) | SERPINA3, AACT, ACT |
| Alpha-actinin-3 deficiency (3) | ACTN3 |
| Alpha-methylacetoacetic aciduria, 203750 | ACAT1 |
| (3) | |
| Alpha-methylacyl-CoA racemase deficiency | AMACR |
| (3) | |
| Alpha-thalassemia/mental retardation | ATRX, XH2, XNP, MRXS3, SHS |
| syndrome, 301040 (3) | |
| Alpha-thalassemia myelodysplasia | ATRX, XH2, XNP, MRXS3, SHS |
| syndrome, somatic, 300448 (3) | |
| Alport syndrome, 301050 (3) | COL4A5, ATS, ASLN |
| Alport syndrome, autosomal recessive, | COL4A3 |
| 203780 (3) | |
| Alport syndrome, autosomal recessive, | COL4A4 |
| 203780 (3) | |
| Alstrom syndrome, 203800 (3) | ALMS1, ALSS, KIAA0328 |
| Alternating hemiplegia of childhood, 104290 | ATP1A2, FHM2, MHP2 |
| (3) | |
| Alveolar soft-part sarcoma, 606243 (3) | ASPCR1, RCC17, ASPL, ASPS |
| Alzheimer disease-1, APP-related (3) | APP, AAA, CVAP, AD1 |
| Alzheimer disease-2, 104310 (3) | APOE, AD2 |
| Alzheimer disease-4, 606889 (3) | PSEN2, AD4, STM2 |
| Alzheimer disease, late-onset, 104300 (3) | APBB2, FE65L1 |
| Alzheimer disease, late-onset, susceptibility | NOS3 |
| to, 104300 (3) | |
| Alzheimer disease, late-onset, susceptibility | PLAU, URK |
| to, 104300 (3) | |
| Alzheimer disease, susceptibility to, 104300 | ACE, DCP1, ACE1 |
| (3) | |
| Alzheimer disease, susceptibility to, 104300 | MPO |
| (3) | |
| Alzheimer disease, susceptibility to, 104300 | PACIP1, PAXIP1L, PTIP |
| (3) | |
| Alzheimer disease, susceptibility to (3) | A2M |
| Alzheimer disease, susceptibility to (3) | BLMH, BMH |
| Alzheimer disease, type 3, 607822 (3) | PSEN1, AD3 |
| Alzheimer disease, type 3, with spastic | PSEN1, AD3 |
| paraparesis and apraxia, 607822 (3) | |
| Alzheimer disease, type 3, with spastic | PSEN1, AD3 |
| paraparesis and unusual plaques, 607822 | |
| (3) | |
| Amelogenesis imperfecta 2, hypoplastic | ENAM |
| local, 104500 (3) | |
| Amelogenesis imperfecta, 301200 (3) | AMELX, AMG, AIH1, AMGX |
| Amelogenesis imperfecta, hypomaturation- | DLX3, TDO |
| hypoplastic type, with taurodontism, 104510 | |
| (3) | |
| Amelogenesis imperfecta, hypoplastic, and | ENAM |
| openbite malocclusion, 608563 (3) | |
| Amelogenesis imperfecta, pigmented | KLK4, EMSP1, PRSS17 |
| hypomaturation type, 204700 (3) | |
| Amish infantile epilepsy syndrome, 609056 | SIAT9, ST3GALV |
| (3) | |
| AMP deaminase deficiency, erythrocytic (3) | AMPD3 |
| Amyloid neuropathy, familial, several allelic | TTR, PALB |
| types (3) | |
| Amyloidosis, 3 or more types (3) | APOA1 |
| Amyloidosis, cerebroarterial, Dutch type (3) | APP, AAA, CVAP, AD1 |
| Amyloidosis, Finnish type, 105120 (3) | GSN |
| Amyloidosis, hereditary renal, 105200 (3) | FGA |
| Amyloidosis, renal, 105200 (3) | LYZ |
| Amyloidosis, senile systemic (3) | TTR, PALB |
| Amyotrophic lateral sclerosis 8, 608627 (3) | VAPB, VAPC, ALS8 |
| Amyotrophic lateral sclerosis, due to SOD1 | SOD1, ALS1 |
| deficiency, 105400 (3) | |
| Amyotrophic lateral sclerosis, juvenile, | ALS2, ALSJ, PLSJ, IAHSP |
| 205100 (3) | |
| Amyotrophic lateral sclerosis, susceptibility | DCTN1 |
| to, 105400 (3) | |
| Amyotrophic lateral sclerosis, susceptibility | NEFH |
| to, 105400 (3) | |
| Amyotrophic lateral sclerosis, susceptibility | PRPH |
| to, 105400 (3) | |
| Analbuminemia (3) | ALB |
| Analgesia from kappa-opioid receptor | MC1R |
| agonist, female-specific (3) | |
| Anderson disease, 607689 (3) | SARA2, SAR1B, CMRD |
| Androgen insensitivity, 300068 (3) | AR, DHTR, TFM, SBMA, KD, SMAX1 |
| Anemia, congenital dyserythropoietic, type I, | CDAN1, CDA1 |
| 224120 (3) | |
| Anemia, Diamond-Blackfan, 105650 (3) | RPS19, DBA |
| Anemia, hemolytic, due to PK deficiency (3) | PKLR, PK1 |
| Anemia, hemolytic, due to UMPH1 | NT5C3, UMPH1, PSN1 |
| deficiency, 266120 (3) | |
| Anemia, hemolytic, Rh-null, regulator type, | RHAG, RH50A |
| 268150 (3) | |
| Anemia, hypochromic microcytic, 206100 | NRAMP2 |
| (3) | |
| Anemia, neonatal hemolytic, fatal and near- | SPTB |
| fatal (3) | |
| Anemia, sideroblastic/hypochromic (3) | ALAS2, ANH1, ASB |
| Anemia, sideroblastic, with ataxia, 301310 | ABCB7, ABC7, ASAT |
| (3) | |
| Aneurysm, familial arterial (3) | COL3A1 |
| Angelman syndrome, 105830 (3) | MECP2, RTT, PPMX, MRX16, MRX79 |
| Angelman syndrome, 105830 (3) | UBE3A, ANCR |
| Angioedema, hereditary, 106100 (3) | C1NH, HAE1, HAE2, SERPING1 |
| Angioedema induced by ACE inhibitors, | XPNPEP2 |
| susceptibility to (3) | |
| Angiofibroma, sporadic (3) | MEN1 |
| Angiotensin I-converting enzyme, benign | ACE, DCP1, ACE1 |
| serum increase (3) | |
| Anhaptoglobinemia (3) | HP |
| Aniridia, type II, 106210 (3) | PAX6, AN2, MGDA |
| Ankylosing spoldylitis, susceptibility to, | HLA-B |
| 106300 (3) | |
| Anophthalmia 3, 206900 (3) | SOX2, ANOP3 |
| Anorexia nervosa, susceptibility to, 606788 | HTR2A |
| (3) | |
| Anterior segment anomalies and cataract | EYA1, BOR |
| (3) | |
| Anterior segment mesenchymal dysgenesis, | FOXE3, FKHL12, ASMD |
| 107250 (3) | |
| Anterior segment mesenchymal dysgenesis | FOXC1, FKHL7, FREAC3 |
| (3) | |
| Anterior segment mesenchymal dysgenesis | PITX3 |
| and cataract, 107250 (3) | |
| Antithrombin III deficiency (3) | AT3 |
| Antley-Bixler syndrome, 207410 (3) | POR |
| Anxiety-related personality traits (3) | SLC6A4, HIT, OCD1 |
| Aortic aneurysm, ascending, and dissection | FBN1, MFS1, WMS |
| (3) | |
| Apert syndrome, 101200 (3) | FGFR2, BEK, CFD1, JWS |
| Aplasia of lacrimal and salivary glands, | FGF10 |
| 180920 (3) | |
| Aplastic anemia, 609135 (3) | IFNG |
| Aplastic anemia, 609135 (3) | TERC, TRC3, TR |
| Aplastic anemia, susceptibility to, 609135 | TERT, TCS1, EST2 |
| (3) | |
| Apnea, postanesthetic (3) | BCHE, CHE1 |
| ApoA-I and apoC-III deficiency, combined | APOA1 |
| (3) | |
| Apolipoprotein A-II deficiency (3) | APOA2 |
| Apolipoprotein C3 deficiency (3) | APOC3 |
| Apolipoprotein H deficiency (3) | APOH |
| Apparent mineralocorticoid excess, | HSD11B2, HSD11K |
| hypertension due to (3) | |
| Aquaporin-1 deficiency (3) | AQP1, CHIP28, CO |
| ARC syndrome, 208085 (3) | VPS33B |
| Argininemia, 207800 (3) | ARG1 |
| Argininosuccinic aciduria, 207900 (3) | ASL |
| Aromatase deficiency (3) | CYP19A1, CYP19, ARO |
| Aromatic L-amino acid decarboxylase | DDC |
| deficiency, 608643 (3) | |
| Arrhythmogenic right ventricular dysplasia 2, | RYR2, VTSIP |
| 600996 (3) | |
| Arrhythmogenic right ventricular dysplasia 8, | DSP, KPPS2, PPKS2 |
| 607450 (3) | |
| Arrhythmogenic right ventricular dysplasia, | PKP2, ARVD9 |
| familial, 9, 609040 (3) | |
| Arthrogryposis multiplex congenita, distal, | TPM2, TMSB, AMCD1, DA1 |
| type 1, 108120 (3) | |
| Arthrogryposis multiplex congenita, distal, | TNNI2, AMCD2B, DA2B, FSSV |
| type 2B, 601680 (3) | |
| Arthropathy, progressive | WISP3, PPAC, PPD |
| pseudorheumatoid, of childhood, 208230 (3) | |
| Arthyrgryposis multiplex congenita, distal, | TNNT3, AMCD2B, DA2B, FSSV |
| type 2B, 601680 (3) | |
| Aspartylglucosaminuria (3) | AGA |
| Asperger syndrome, 300494 (3) | NLGN3 |
| Asperger syndrome, 300497 (3) | NLGN4, KIAA1260, AUTSX2 |
| Asthma, 600807 (3) | PHF11, NYREN34 |
| Asthma, atopic, susceptibility to (3) | MS4A2, FCER1B |
| Asthma, dimished response to | ALOX5 |
| antileukotriene treatment in, 600807 (3) | |
| Asthma, nocturnal, susceptibility to (3) | ADRB2 |
| Asthma, susceptibility to, 1, 607277 (3) | PTGDR, AS1 |
| Asthma, susceptibility to, 2, 608584 (3) | GPR154, GPRA, VRR1, PGR14 |
| Asthma, susceptibility to (3) | HNMT |
| Asthma, susceptibility to, 600807 (3) | IL12B, NKSF2 |
| Asthma, susceptibility to, 600807 (3) | IL13, ALRH |
| Asthma, susceptibility to, 600807 (3) | PLA2G7, PAFAH |
| Asthma, susceptibility to, 600807 (3) | SCGB3A2, UGRP1 |
| Asthma, susceptibility to, 600807 (3) | TNF, TNFA |
| Asthma, susceptibility to, 600807 (3) | UGB, CC10, CCSP, SCGB1A1 |
| Ataxia, cerebellar, Cayman type, 601238 (3) | ATCAY, CLAC, KIAA1872 |
| Ataxia, early-onset, with oculomotor apraxia | APTX, AOA, AOA1 |
| and hypoalbuminemia, 208920 (3) | |
| Ataxia, episodic (3) | CACNB4, EJM |
| Ataxia-ocular apraxia-2, 606002 (3) | SETX, SCAR1, AOA2 |
| Ataxia-telangiectasia, 208900 (3) | ATM, ATA, AT1 |
| Ataxia-telangiectasia-like disorder, 604391 | MRE11A, MRE11, ATLD |
| (3) | |
| Ataxia with isolated vitamin E deficiency, | TTPA, TTP1, AVED |
| 277460 (3) | |
| Atelosteogenesis II, 256050 (3) | SLC26A2, DTD, DTDST, D5S1708, |
| | EDM4 |
| Atelostogenesis, type I, 108720 (3) | FLNB, SCT, AOI |
| Athabaskan brainstem dysgenesis | HOXA1, HOX1F, BSAS |
| syndrome, 601536 (3) | |
| Atherosclerosis, susceptibility to (3) | ALOX5 |
| Atopy, 147050 (3) | SPINK5, LEKTI |
| Atopy, resistance to, 147050 (3) | HAVCR1, HAVCR |
| Atopy, susceptibility to, 147050 (3) | PLA2G7, PAFAH |
| Atopy, susceptibility to, 147050 (3) | SELP, GRMP |
| Atopy, susceptibility to (3) | IL4R, IL4RA |
| Atransferrinemia, 209300 (3) | TF |
| Atrial fibrillation, familial, 607554 (3) | KCNE2, MIRP1, LQT6 |
| Atrial fibrillation, familial, 607554 (3) | KCNQ1, KCNA9, LQT1, KVLQT1, |
| | ATFB1 |
| Atrial septal defect-2, 607941 (3) | GATA4 |
| Atrial septal defect 3 (3) | MYH6, ASD3, MYHCA |
| Atrial septal defect with atrioventricular | NKX2E, CSX |
| conduction defects, 108900 (3) | |
| Atrichia with papular lesions, 209500 (3) | HR, AU |
| Atrioventricular block, idiopathic second- | NKX2E, CSX |
| degree (3) | |
| Atrioventricular septal defect, 600309 (3) | GJA1, CX43, ODDD, SDTY3, ODOD |
| Atrioventricular septal defect, partial, with | CRELD1, AVSD2 |
| heterotaxy syndrome, 606217 (3) | |
| Atrioventricular septal defect, susceptibility | CRELD1, AVSD2 |
| to, 2, 606217 (3) | |
| Attention deficit-hyperactivity disorder, | DRD5, DRD1B, DRD1L2 |
| susceptibility to, 143465 (3) | |
| Autism, susceptibility to, 209850 (3) | GLO1 |
| Autism, X-linked, 300425 (3) | MECP2, RTT, PPMX, MRX16, MRX79 |
| Autism, X-linked, 300425 (3) | NLGN3 |
| Autism, X-linked, 300495 (3) | NLGN4, KIAA1260, AUTSX2 |
| Autoimmune lymphoproliferative syndrome, | TNFRSF6, APT1, FAS, CD95, ALPS1A |
| 601859 (3) | |
| Autoimmune lymphoproliferative syndrome, | TNFRSF6, APT1, FAS, CD95, ALPS1A |
| type IA, 601859 (3) | |
| Autoimmune lymphoproliferative syndrome, | CASP10, MCH4, ALPS2 |
| type II, 603909 (3) | |
| Autoimmune lymphoproliferative syndrome, | CASP8, MCH5 |
| type IIB, 607271 (3) | |
| Autoimmune polyglandular disease, type I, | AIRE, APECED |
| 240300 (3) | |
| Autoimmune thyroid disease, susceptibility | TG, AITD3 |
| to 3, 608175 (3) | |
| Autonomic nervous system dysfunction (3) | DRD4 |
| Axenfeld anomaly (3) | FOXC1, FKHL7, FREAC3 |
| Azoospermia (3) | USP9Y, DFFRY |
| Azoospermia due to perturbations of | SYCP3, SCP3, COR1 |
| meiosis, 270960 (3) | |
| Bamforth-Lazarus syndrome, 241850 (3) | FOXE1, FKHL15, TITF2, TTF2 |
| Bannayan-Riley-Ruvalcaba syndrome, | PTEN, MMAC1 |
| 153480 (3) | |
| Bannayan-Zonana syndrome, 153480 (3) | PTEN, MMAC1 |
| Bardet-Biedl syndrome 1, 209900 (3) | BBS1 |
| Bardet-Biedl syndrome 1, modifier of, | ARL6, BBS3 |
| 209900 (3) | |
| Bardet-Biedl syndrome, 209900 (3) | BBS7 |
| Bardet-Biedl syndrome 2, 209900 (3) | BBS2 |
| Bardet-Biedl syndrome 3, 600151 (3) | ARL6, BBS3 |
| Bardet-Biedl syndrome 4, 209900 (3) | BBS4 |
| Bardet-Biedl syndrome 5, 209900 (3) | BBS5 |
| Bardet-Biedl syndrome 6, 209900 (3) | MKKS, HMCS, KMS, MKS, BBS6 |
| Bardet-Biedl syndrome 8, 209900 (3) | TTC8, BBS8 |
| Bare lymphocyte syndrome, type I, 604571 | TAPBP, TPSN |
| (3) | |
| Bare lymphocyte syndrome, type I, due to | TAP2, ABCB3, PSF2, RING11 |
| TAP2 deficiency, 604571 (3) | |
| Bare lymphocyte syndrome, type II, | MHC2TA, C2TA |
| complementation group A, 209920 (3) | |
| Bare lymphocyte syndrome, type II, | RFX5 |
| complementation group C, 209920 (3) | |
| Bare lymphocyte syndrome, type II, | RFXAP |
| complementation group D, 209920 (3) | |
| Bare lymphocyte syndrome, type II, | RFX5 |
| complementation group E, 209920 (3) | |
| Barth syndrome, 302060 (3) | TAZ, EFE2, BTHS, CMD3A, LVNCX |
| Bart-Pumphrey syndrome, 149200 (3) | GJB2, CX26, DFNB1, PPK, DFNA3, |
| | KID, HID |
| Bartter syndrome, type 1, 601678 (3) | SLC12A1, NKCC2 |
| Bartter syndrome, type 2, 241200 (3) | KCNJ1, ROMK1 |
| Bartter syndrome, type 3, 607364 (3) | CLCNKB |
| Bartter syndrome, type 4, 602522 (3) | BSND |
| Bartter syndrome, type 4, digenic, 602522 | CLCNKA |
| (3) | |
| Bartter syndrome, type 4, digenic, 602522 | CLCNKB |
| (3) | |
| Basal cell carcinoma (3) | RASA1, GAP, CMAVM, PKWS |
| Basal cell carcinoma, somatic, 605462 (3) | PTCH2 |
| Basal cell carcinoma, somatic, 605462 (3) | PTCH, NBCCS, BCNS, HPE7 |
| Basal cell carcinoma, sporadic (3) | SMOH, SMO |
| Basal cell nevus syndrome, 109400 (3) | PTCH, NBCCS, BCNS, HPE7 |
| Basal ganglia disease, adult-onset, 606159 | FTL |
| (3) | |
| Basal ganglia disease, biotin-responsive, | SLC19A3 |
| 607483 (3) | |
| B-cell non-Hodgkin lymphoma, high-grade | BCL7A, BCL7 |
| (3) | |
| BCG infection, generalized familial (3) | IFNGR1 |
| Beare-Stevenson cutis gyrata syndrome, | FGFR2, BEK, CFD1, JWS |
| 123790 (3) | |
| Becker muscular dystrophy, 300376 (3) | DMD, BMD |
| Becker muscular dystrophy modifier, | MYF6 |
| 310200 (3) | |
| Beckwith-Wiedemann syndrome, 130650 | CDKN1C, KIP2, BWS |
| (3) | |
| Beckwith-Wiedemann syndrome, 130650 | H19, D11S813E, ASM1, BWS |
| (3) | |
| Beckwith-Wiedemann syndrome, 130650 | KCNQ10T1, LIT1 |
| (3) | |
| Beckwith-Wiedemann syndrome, 130650 | NSD1, ARA267, STO |
| (3) | |
| Benzene toxicity, susceptibility to (3) | NQO1, DIA4, NMOR1 |
| Bernard-Soulier syndrome, 231200 (3) | GP1BA |
| Bernard-Soulier syndrome, type B, 231200 | GP1BB |
| (3) | |
| Bernard-Soulier syndrome, type C (3) | GP9 |
| Beryllium disease, chronic, susceptibility to | HLA-DPB1 |
| (3) | |
| Beta-2-adrenoreceptor agonist, reduced | ADRB2 |
| response to (3) | |
| Beta-ureidopropionase deficiency (3) | UPB1, BUP1 |
| Bethlem myopathy, 158810 (3) | COL6A1, OPLL |
| Bethlem myopathy, 158810 (3) | COL6A2 |
| Bethlem myopathy, 158810 (3) | COL6A3 |
| Bietti crystalline corneoretinal dystrophy, | CYP4V2, BCD |
| 210370 (3) | |
| Bile acid malabsorption, primary (3) | SLC10A2, NTCP2 |
| Biotinidase deficiency, 253260 (3) | BTD |
| Bipolar disorder, susceptibility to, 125480 | XBP1, XBP2 |
| (3) | |
| Birt-Hogg-Dube syndrome, 135150 (3) | FLCN, BHD |
| Bladder cancer, 109800 (3) | FGFR3, ACH |
| Bladder cancer, 109800 (3) | KRAS2, RASK2 |
| Bladder cancer, 109800 (3) | RB1 |
| Bladder cancer, somatic, 109800 (3) | HRAS |
| Blau syndrome, 186580 (3) | CARD15, NOD2, IBD1, CD, ACUG, |
| | PSORAS1 |
| Bleeding disorder due to defective | TBXA2R |
| thromboxane A2 receptor (3) | |
| Bleeding due to platelet ADP receptor | P2RX1, P2X1 |
| defect, 600515 (3) | |
| Blepharophimosis, epicanthus inversus, and | FOXL2, BPES, BPES1, PFRK, POF3 |
| ptosis, type 1, 110100 (3) | |
| Blepharophimosis, epicanthus inversus, and | FOXL2, BPES, BPES1, PFRK, POF3 |
| ptosis, type 2, 110100 (3) | |
| Blepharospasm, primary benign, 606798 (3) | DRD5, DRD1B, DRD1L2 |
| Blood group, ABO system (3) | ABO |
| Blood group, Auberger system (3) | LU, AU, BCAM |
| Blood group, Colton, 110450 (3) | AQP1, CHIP28, CO |
| Blood group Cromer (3) | DAF |
| Blood group, Diego, 110500 (3) | SLC4A1, AE1, EPB3 |
| Blood group, Dombrock (3) | ART4, DO |
| Blood group, Gerbich (3) | GYPC, GE, GPC |
| Blood group GIL, 607457 (3) | AQP3 |
| Blood group, Ii, 110800 (3) | GCNT2 |
| Blood group, Indian system (3) | CD44, MDU2, MDU3, MIC4 |
| Blood group, Kell (3) | KEL |
| Blood group, Kidd (3) | SLC14A1, JK, UTE, UT1 |
| Blood group, Knops system, 607486 (3) | CR1, C3BR |
| Blood group, Landsteiner-Wiener (3) | LW |
| Blood group, Lewis (3) | FUT3, LE |
| Blood group, Lutheran system (3) | LU, AU, BCAM |
| Blood group, MN (3) | GYPA, MN, GPA |
| Blood group, OK, 111380 (3) | BSG |
| Blood group, P system, 111400 (3) | A4GALT, PK |
| Blood group, P system, 111400 (3) | B3GALT3, GLCT3, P |
| Blood group, Rhesus (3) | RHCE |
| Blood group, Ss (3) | GYPB, SS, MNS |
| Blood group, Waldner, 112010 (3) | SLC4A1, AE1, EPB3 |
| Blood group, Wright, 112050 (3) | SLC4A1, AE1, EPB3 |
| Blood group, XG system (3) | XG |
| Blood group, Yt system, 112100 (3) | ACHE, YT |
| Bloom syndrome, 210900 (3) | RECQL3, RECQ2, BLM, BS |
| Blue-cone monochromacy, 303700 (3) | OPN1LW, RCP, CBP, CBBM |
| Blue-cone monochromacy, 303700 (3) | OPN1MW, GCP, CBD, CBBM |
| Bombay phenotype (3) | FUT1, H, HH |
| Bombay phenotype (3) | FUT2, SE |
| Bone mineral density variability 1, 601884 | LRP5, BMND1, LRP7, LR3, OPPG, |
| (3) | VBCH2 |
| Borjeson-Forssman-Lehmann syndrome, | PHF6, BFLS |
| 301900 (3) | |
| Bosley-Salih-Alorainy syndrome, 601536 (3) | HOXA1, HOX1F, BSAS |
| Bothnia retinal dystrophy, 607475 (3) | RLBP1 |
| Brachydactyly, type A1, 112500 (3) | IHH, BDA1 |
| Brachydactyly, type A2, 112600 (3) | BMPR1B, ALK6 |
| Brachydactyly, type B1, 113000 (3) | ROR2, BDB1, BDB, NTRKR2 |
| Brachydactyly, type C, 113100 (3) | GDF5, CDMP1 |
| Brachydactyly, type D, 113200 (3) | HOXD13, HOX4I, SPD |
| Brachydactyly, type E, 113300 (3) | HOXD13, HOX4I, SPD |
| Bradyopsia, 608415 (3) | R9AP, RGS9, PERRS |
| Bradyopsia, 608415 (3) | RGS9, PERRS |
| Branchiootic syndrome (3) | EYA1, BOR |
| Branchiootorenal syndrome, 113650 (3) | EYA1, BOR |
| Branchiootorenal syndrome with cataract, | EYA1, BOR |
| 113650 (3) | |
| Breast and colorectal cancer, susceptibility | CHEK2, RAD53, CHK2, CDS1, LFS2 |
| to (3) | |
| Breast cancer, 114480 (3) | PIK3CA |
| Breast cancer, 114480 (3) | PPM1D, WIP1 |
| Breast cancer, 114480 (3) | SLC22A1L, BWSCR1A, IMPT1 |
| Breast cancer, 114480 (3) | TP53, P53, LFS1 |
| Breast cancer-1 (3) | BRCA1, PSCP |
| Breast cancer 2, early onset (3) | BRCA2, FANCD1 |
| Breast cancer (3) | TSG101 |
| Breast cancer, early-onset, 114480 (3) | BRIP1, BACH1, FANCJ |
| Breast cancer, invasive intraductal (3) | RAD54L, HR54, HRAD54 |
| Breast cancer, lobular (3) | CDH1, UVO |
| Breast cancer, male, susceptibility to, | BRCA2, FANCD1 |
| 114480 (3) | |
| Breast cancer, male, with Reifenstein | AR, DHTR, TFM, SBMA, KD, SMAX1 |
| syndrome (3) | |
| Breast cancer, somatic, 114480 (3) | KRAS2, RASK2 |
| Breast cancer, somatic, 114480 (3) | RB1CC1, CC1, KIAA0203 |
| Breast cancer, sporadic (3) | PHB |
| Breast cancer, susceptibility to, 114480 (3) | ATM, ATA, AT1 |
| Breast cancer, susceptibility to, 114480 (3) | BARD1 |
| Breast cancer, susceptibility to, 114480 (3) | CHEK2, RAD53, CHK2, CDS1, LFS2 |
| Breast cancer, susceptibility to, 114480 (3) | RAD51A, RECA |
| Breast cancer, susceptibility to (3) | XRCC3 |
| Breast-ovarian cancer (3) | BRCA1, PSCP |
| Brody myopathy, 601003 (3) | ATP2A1, SERCA1 |
| Bruck syndrome 2, 609220 (3) | PLOD2 |
| Brugada syndrome, 601144 (3) | SCN5A, LQT3, IVF, HB1, SSS1 |
| Brunner syndrome (3) | MAOA |
| Burkitt lymphoma, 113970 (3) | MYC |
| Buschke-Ollendorff syndrome, 166700 (3) | LEMD3, MAN1 |
| Butterfly dystrophy, retinal, 169150 (3) | RDS, RP7, PRPH2, PRPH, AVMD, |
| | AOFMD |
| C1q deficiency, type A (3) | C1QA |
| C1q deficiency, type B (3) | C1QB |
| C1q deficiency, type C (3) | C1QG |
| C1s deficiency, isolated (3) | C1S |
| C2 deficiency (3) | C2 |
| C3b inactivator deficiency (3) | IF |
| C3 deficiency (3) | C3 |
| C4 deficiency (3) | C4A, C4S |
| C4 deficiency (3) | C4B, C4F |
| C6 deficiency (3) | C6 |
| C7 deficiency (3) | C7 |
| C8 deficiency, type II (3) | C8B |
| C9 deficiency (3) | C9 |
| C9 deficiency with dermatomyositis (3) | C9 |
| Cafe-au-lait spots, multiple, with leukemia, | MSH2, COCA1, FCC1, HNPCC1 |
| 114030 (3) | |
| Cafe-au-lait spots with glioma or leukemia, | MLH1, COCA2, HNPCC2 |
| 114030 (3) | |
| Caffey disease, 114000 (3) | COL1A1 |
| Calcinosis, tumoral, 211900 (3) | FGF23, ADHR, HPDR2, PHPTC |
| Calcinosis, tumoral, 211900 (3) | GALNT3 |
| Campomelic dysplasia, 114290 (3) | SOX9, CMD1, SRA1 |
| Campomelic dysplasia with autosomal sex | SOX9, CMD1, SRA1 |
| reversal, 114290 (3) | |
| Camptodactyly-arthropathy-coxa vara- | PRG4, CACP, MSF, SZP, HAPO |
| pericarditis syndrome, 208250 (3) | |
| Camurati-Engelmann disease, 131300 (3) | TGFB1, DPD1, CED |
| Canavan disease, 271900 (3) | ASPA |
| Cancer progression/metastasis (3) | FGFR4 |
| Cancer susceptibility (3) | MSH6, GTBP, HNPCC5 |
| Capillary malformation-arteriovenous | RASA1, GAP, CMAVM, PKWS |
| malformation, 608354 (3) | |
| Carbamoylphosphate synthetase I | CPS1 |
| deficiency, 237300 (3) | |
| Carbohydrate-deficient glycoprotein | PMM2, CDG1 |
| syndrome, type I, 212065 (3) | |
| Carbohydrate-deficient glycoprotein | MPI, PMI1 |
| syndrome, type Ib, 602579 (3) | |
| Carbohydrate-deficient glycoprotein | MGAT2, CDGS2 |
| syndrome, type II, 212066 (3) | |
| Carboxypeptidase N deficiency, 212070 (3) | CPN1, SCPN, CPN |
| Carcinoid tumor of lung (3) | MEN1 |
| Carcinoid tumors, intestinal, 114900 (3) | SDHD, PGL1 |
| Cardioencephalomyopathy, fatal infantile, | SCO2 |
| due to cytochrome c oxidase deficiency, | |
| 604377 (3) | |
| Cardiomyopathy, Familial hypertrophic, 8, | MYL3, CMH8 |
| 608751 (3) | |
| Cardiomyopathy, dilated, 115200 (3) | ACTC |
| Cardiomyopathy, dilated, 115200 (3) | MYH7, CMH1, MPD1 |
| Cardiomyopathy, dilated, 1A, 115200 (3) | LMNA, LMN1, EMD2, FPLD, CMD1A, |
| | HGPS, LGMD1B |
| Cardiomyopathy, dilated, 1D, 601494 (3) | TNNT2, CMH2, CMD1D |
| Cardiomyopathy, dilated, 1G, 604145 (3), | TTN, CMD1G, TMD, LGMD2J |
| Tibial muscular dystrophy, tardive, 600334 | |
| (3) | |
| Cardiomyopathy, dilated, 1I, 604765 (3) | DES, CMD1I |
| Cardiomyopathy, dilated, 1J, 605362 (3) | EYA4, DFNA10, CMD1J |
| Cardiomyopathy, dilated, 1L, 606685 (3) | SGCD, SGD, LGMD2F, CMD1L |
| Cardiomyopathy, dilated, 1M, 607482 (3) | CSRP3, CRP3, CLP, CMD1M |
| Cardiomyopathy, dilated, 1N, 607487 (3) | TCAP, LGMD2G, CMD1N |
| Cardiomyopathy, dilated, with ventricular | ABCC9, SUR2 |
| tachycardia, 608569 (3) | |
| Cardiomyopathy, dilated, X-linked, 302045 | DMD, BMD |
| (3) | |
| Cardiomyopathy, familial hypertrophic, 10, | MYL2, CMH10 |
| 608758 (3) | |
| Cardiomyopathy, familial hypertrophic, 1, | MYH7, CMH1, MPD1 |
| 192600 (3) | |
| Cardiomyopathy, familial hypertrophic, | ACTC |
| 192600 (3) | |
| Cardiomyopathy, familial hypertrophic, | CAV3, LGMD1C |
| 192600 (3) | |
| Cardiomyopathy, familial hypertrophic, | MYH6, ASD3, MYHCA |
| 192600 (3) | |
| Cardiomyopathy, familial hypertrophic, | TNNC1 |
| 192600 (3) ( ) | |
| Cardiomyopathy, familial hypertrophic, 2, | TNNT2, CMH2, CMD1D |
| 115195 (3) | |
| Cardiomyopathy, familial hypertrophic, 3, | TPM1, CMH3 |
| 115196 (3) | |
| Cardiomyopathy, familial hypertrophic (3) | TNNI3 |
| Cardiomyopathy, familial hypertrophic, 4, | MYBPC3, CMH4 |
| 115197 (3) | |
| Cardiomyopathy, familial hypertrophic, 9 (3) | TTN, CMD1G, TMD, LGMD2J |
| Cardiomyopathy, familial restrictive, 115210 | TNNI3 |
| (3) | |
| Cardiomyopathy, hypertrophic, early-onset | COX15 |
| fatal (3) | |
| Cardiomyopathy, hypertrophic, mid-left | MYL2, CMH10 |
| ventricular chamber type, 608758 (3) | |
| Cardiomyopathy, hypertrophic, | MYLK2, MLCK |
| midventricular, digenic, 192600 (3) | |
| Cardiomyopathy, hypertrophic, with WPW, | PRKAG2, WPWS |
| 600858 (3) | |
| Cardiomyopathy, idiopathic dilated, 115200 | PLN, PLB |
| (3) | |
| Cardiomyopathy, X-linked dilated, 300069 | TAZ, EFE2, BTHS, CMD3A, LVNCX |
| (3) | |
| Carney complex, type 1, 160980 (3) | PRKAR1A, TSE1, CNC1, CAR |
| Carney complex variant, 608837 (3) | MYH8 |
| Carnitine-acylcarnitine translocase | SLC25A20, CACT, CAC |
| deficiency (3) | |
| Carnitine deficiency, systemic primary, | SLC22A5, OCTN2, CDSP, SCD |
| 212140 (3) | |
| Carpal tunnel syndrome, familial (3) | TTR, PALB |
| Cartilage-hair hypoplasia, 250250 (3) | RMRP, RMRPR, CHH |
| Cataract, autosomal dominant nuclear (3) | CRYAA, CRYA1 |
| Cataract, cerulean, type 2, 601547 (3) | CRYBB2, CRYB2 |
| Cataract, congenital (3) | PITX3 |
| Cataract, congenital, 604219 (3) | BFSP2, CP49, CP47 |
| Cataract, congenital progressive, autosomal | CRYAA, CRYA1 |
| recessive (3) | |
| Cataract, congenital, with late-onset corneal | PAX6, AN2, MGDA |
| dystrophy (3) | |
| Cataract, congenital zonular, with sutural | CRYBA1, CRYB1 |
| opacities, 600881 (3) | |
| Cataract, Coppock-like, 604307 (3) | CRYGC, CRYG3, CCL |
| Cataract, cortical pulverulent, late-onset (3) | LIM2, MP19 |
| Cataract, crystalline aculeiform, 115700 (3) | CRYGD, CRYG4 |
| Cataract, juvenile-onset, 604219 (3) | BFSP2, CP49, CP47 |
| Cataract, lamellar, 116800 (3) | HSF4, CTM |
| Cataract, Marner type, 116800 (3) | HSF4, CTM |
| Cataract, polymorphic and lamellar, 604219 | MIP, AQP0 |
| (3) | |
| Cataract, posterior polar 2 (3) | CRYAB, CRYA2, CTPP2 |
| Cataract, pulverulent (3) | CRYBB1 |
| Cataracts, punctate, progressive juvenile- | CRYGD, CRYG4 |
| onset (3) | |
| Cataract, sutural, with punctate and | CRYBB2, CRYB2 |
| cerulean opacities, 607133 (3) | |
| Cataract, variable zonular pulverulent (3) | CRYGC, CRYG3, CCL |
| Cataract, zonular central nuclear, autosomal | CRYAA, CRYA1 |
| dominant (3) | |
| Cataract, zonular pulverulent-1, 116200 (3) | GJA8, CX50, CAE1 |
| Cataract, zonular pulverulent-3, 601885 (3) | GJA3, CX46, CZP3, CAE3 |
| Cavernous malformations of CNS and | CCM1, CAM, KRIT1 |
| retina, 116860 (3) | |
| CD59 deficiency (3) | CD59, MIC11 |
| CD8 deficiency, familial, 608957 (3) | CD8A |
| Central core disease, 117000 (3) | RYR1, MHS, CCO |
| Central core disease, one form (3) ( ) | MYH7, CMH1, MPD1 |
| Central hypoventilation syndrome, 209880 | GDNF |
| (3) | |
| Central hypoventilation syndrome, | BDNF |
| congenital, 209880 (3) | |
| Central hypoventilation syndrome, | EDN3 |
| congenital, 209880 (3) | |
| Central hypoventilation syndrome, | PMX2B, NBPHOX, PHOX2B |
| congenital, 209880 (3) | |
| Central hypoventilation syndrome, | RET, MEN2A |
| congenital, 209880 (3) | |
| Cerebellar ataxia, 604290 (3) | CP |
| Cerebellar ataxia, pure (3) | CACNA1A, CACNL1A4, SCA6 |
| Cerebellar hypoplasia, VLDLR-associated, | VLDLR, VLDLRCH |
| 224050 (3) | |
| Cerebral amyloid angiopathy, 105150 (3) | ABCA1, ABC1, HDLDT1, TGD |
| Cerebral amyloid angiopathy, 105150 (3) | CST3 |
| Cerebral arteriopathy with subcortical | NOTCH3, CADASIL, CASIL |
| infarcts and leukoencephalopathy, 125310 | |
| (3) | |
| Cerebral cavernous malformations-1, | CCM1, CAM, KRIT1 |
| 116860 (3) | |
| Cerebral cavernous malformations-2, | C7orf22, CCM2, MGC4067 |
| 603284 (3) | |
| Cerebral cavernous malformations 3, | PDCD10, TFAR15, CCM3 |
| 603285 (3) | |
| Cerebral dysgenesis, neuropathy, | SNAP29, CEDNIK |
| ichthyosis, and palmoplantar keratoderma | |
| syndrome, 609528 (3) | |
| Cerebrooculofacioskeletal syndrome, | ERCC2, EM9 |
| 214150 (3) | |
| Cerebrooculofacioskeletal syndrome, | ERCC5, XPG |
| 214150 (3) | |
| Cerebrooculofacioskeletal syndrome | ERCC6, CKN2, COFS, CSB |
| 214150 (3) | |
| Cerebrotendinous xanthomatosis, 213700 | CYP27A1, CYP27, CTX |
| (3) | |
| Cerebrovascular disease, occlusive (3) | SERPINA3, AACT, ACT |
| Ceroid lipofuscinosis, neuronal-1, infantile, | PPT1, CLN1 |
| 256730 (3) | |
| Ceroid-lipofuscinosis, neuronal 2, classic | CLN2 |
| late infantile, 204500 (3) | |
| Ceroid-lipofuscinosis, neuronal-3, juvenile, | CLN3, BTS |
| 204200 (3) | |
| Ceroid-lipofuscinosis, neuronal-5, variant | CLN5 |
| late infantile, 256731 (3) | |
| Ceroid-lipofuscinosis, neuronal-6, variant | CLN6 |
| late infantile, 601780 (3) | |
| Ceroid lipofuscinosis, neuronal 8, 600143 | CLN8, EPMR |
| (3) | |
| Ceroid lipofuscinosis, neuronal, variant | PPT1, CLN1 |
| juvenile type, with granular osmiophilic | |
| deposits (3) | |
| Cervical cancer, somatic, 603956 (3) | FGFR3, ACH |
| CETP deficiency, 607322 (3) | CETP |
| Chanarin-Dorfman syndrome, 275630 (3) | ABHD5, CGI58, IECN2, NCIE2 |
| Charcot-Marie-Tooth disease, axonal, type | HSPB1, HSP27, CMT2F |
| 2F, 606595 (3) | |
| Charcot-Marie-Tooth disease, dominant | MPZ, CMT1B, CMTDI3, CHM, DSS |
| intermediate 3, 607791 (3) | |
| Charcot-Marie-Tooth disease, dominant | DNM2 |
| intermediate B, 606482 (3) | |
| Charcot-Marie-Tooth disease, foot deformity | HOXD10, HOX4D |
| of (3) | |
| Charcot-Marie-Tooth disease, mixed axonal | GDAP1, CMT4A, CMT2K, CMT2G |
| and demyelinating type, 214400 (3) | |
| Charcot-Marie-Tooth disease, type 1A, | PMP22, CMT1A, CMT1E, DSS |
| 118220 (3) | |
| Charcot-Marie-Tooth disease, type 1B, | MPZ, CMT1B, CMTDI3, CHM, DSS |
| 118200 (3) | |
| Charcot-Marie-Tooth disease, type 1C, | LITAF, CMT1C |
| 601098 (3) | |
| Charcot-Marie-Tooth disease, type 1D, | EGR2, KROX20 |
| 607678 (3) | |
| Charcot-Marie-Tooth disease, type 1E, | PMP22, CMT1A, CMT1E, DSS |
| 118300 (3) | |
| Charcot-Marie-Tooth disease, type 1F, | NEFL, CMT2E, CMT1F |
| 607734 (3) | |
| Charcot-Marie-Tooth disease, type 2A1, | KIF1B, CMT2A, CMT2A1 |
| 118210 (3) | |
| Charcot-Marie-Tooth disease, type 2A2, | MFN2, KIAA0214, CMT2A2 |
| 609260 (3) | |
| Charcot-Marie-Tooth disease, type 2B, | RAB7, CMT2B, PSN |
| 600882 (3) | |
| Charcot-Marie-Tooth disease, type 2D, | GARS, SMAD1, CMT2D |
| 601472 (3) | |
| Charcot-Marie-Tooth disease, type 2E, | NEFL, CMT2E, CMT1F |
| 607684 (3) | |
| Charcot-Marie-Tooth disease, type 2G, | GDAP1, CMT4A, CMT2K, CMT2G |
| 607706 (3) | |
| Charcot-Marie-Tooth disease, type 2I, | MPZ, CMT1B, CMTDI3, CHM, DSS |
| 607677 (3) | |
| Charcot-Marie-Tooth disease, type 2J, | MPZ, CMT1B, CMTDI3, CHM, DSS |
| 607736 (3) | |
| Charcot-Marie-Tooth disease, type 2K, | GDAP1, CMT4A, CMT2K, CMT2G |
| 607831 (3) | |
| Charcot-Marie-Tooth disease, type 4A, | GDAP1, CMT4A, CMT2K, CMT2G |
| 214400 (3) | |
| Charcot-Marie-Tooth disease, type 4B1, | MTMR2, CMT4B1 |
| 601382 (3) | |
| Charcot-Marie-Tooth disease, type 4B2, | SBF2, MTMR13, CMT4B2 |
| 604563 (3) | |
| Charcot-Marie-Tooth disease, type 4B2, | SBF2, MTMR13, CMT4B2 |
| with early-onset glaucoma, 607739 (3) | |
| Charcot-Marie-Tooth disease, type 4C, | KIAA1985 |
| 601596 (3) | |
| Charcot-Marie-Tooth disease, type 4D, | NDRG1, HMSNL, CMT4D |
| 601455 (3) | |
| Charcot-Marie-Tooth neuropathy, X-linked | GJB1, CX32, CMTX1 |
| dominant, 1, 302800 (3) | |
| CHARGE syndrome, 214800 (3) | CHD7 |
| Char syndrome, 169100 (3) | TFAP2B, CHAR |
| Chediak-Higashi syndrome, 214500 (3) | CHS1, LYST |
| Cherubism, 118400 (3) | SH3BP2, CRPM |
| CHILD syndrome, 308050 (3) | NSDHL |
| Chitotriosidase deficiency (3) | CHIT |
| Chloride diarrhea, congenital, Finnish type, | SLC26A3, DRA, CLD |
| 214700 (3) | |
| Cholelithiasis, 600803 (3) | ABCB4, PGY3, MDR3 |
| Cholestasis, benign recurrent intrahepatic, | ATP8B1, FIC1, BRIC, PFIC1 |
| 243300 (3) | |
| Cholestasis, familial intrahepatic, of | ABCB4, PGY3, MDR3 |
| pregnancy, 147480 (3) | |
| Cholestasis, progressive familial | ATP8B1, FIC1, BRIC, PFIC1 |
| intrahepatic 1, 211600 (3) | |
| Cholestasis, progressive familial | ABCB11, BSEP, SPGP, PFIC2 |
| intrahepatic 2, 601847 (3) | |
| Cholestasis, progressive familial | ABCB4, PGY3, MDR3 |
| intrahepatic 3, 602347 (3) | |
| Cholestasis, progressive familial | HSD3B7, PFIC4 |
| intrahepatic 4, 607765 (3) | |
| Cholesteryl ester storage disease (3) | LIPA |
| Chondrocalcinosis 2, 118600 (3) | ANKH, HANK, ANK, CMDJ, CCAL2, |
| | CPPDD |
| Chondrodysplasia, Grebe type, 200700 (3) | GDF5, CDMP1 |
| Chondrodysplasia punctata, rhizomelic, type | GNPAT, DHAPAT |
| 2, 222765 (3) | |
| Chondrodysplasia punctata, X-linked | EBP, CDPX2, CPXD, CPX |
| dominant, 302960 (3) | |
| Chondrodysplasia punctata, X-linked | ARSE, CDPX1, CDPXR |
| recessive, 302950 (3) | |
| Chondrosarcoma, 215300 (3) | EXT1 |
| Chondrosarcoma, extraskeletal myxoid (3) | CSMF |
| Chondrosarcoma, extraskeletal myxoid (3) | EWSR1, EWS |
| Chorea, hereditary benign, 118700 (3) | TITF1, NKX2A, TTF1 |
| Choreoacanthocytosis, 200150 (3) | VPS13A, CHAC |
| Choreoathetosis, hypothyroidism, and | TITF1, NKX2A, TTF1 |
| respiratory distress (3) | |
| Choroideremia, 303100 (3) | CHM, TCD |
| Chromosome 22q13.3 deletion syndrome, | PSAP2, PROSAP2, KIAA1650 |
| 606232 (3) | |
| Chronic granulomatous disease, autosomal, | CYBA |
| due to deficiency of CYBA, 233690 (3) | |
| Chronic granulomatous disease due to | NCF1 |
| deficiency of NCF-1, 233700 (3) | |
| Chronic granulomatous disease due to | NCF2 |
| deficiency of NCF-2, 233710 (3) | |
| Chronic granulomatous disease, X-linked, | CYBB, CGD |
| 306400 (3) | |
| Chronic infections, due to opsonin defect (3) | MBL2, MBL, MBP1 |
| Chudley-Lowry syndrome, 309490 (3) | ATRX, XH2, XNP, MRXS3, SHS |
| Chylomicronemia syndrome, familial (3) | LPL, LIPD |
| Chylomicron retention disease, 246700 (3) | SARA2, SAR1B, CMRD |
| Chylomicron retention disease with | SARA2, SAR1B, CMRD |
| Marinesco-Sjogren syndrome, 607692 (3) | |
| Ciliary dyskinesia, primary, 1, 242650 (3) | DNAI1, CILD1, ICS, PCD |
| Ciliary dyskinesia, primary, 3 608644 (3) | DNAH5, HL1, PCD, CILD3 |
| CINCA syndrome, 607115 (3) | CIAS1, C1orf7, FCU, FCAS |
| Cirrhosis, cryptogenic (3) | KRT18 |
| Cirrhosis, cryptogenic (3) | KRT8 |
| Cirrhosis, noncryptogenic, susceptibility to, | KRT18 |
| 215600 (3) | |
| Cirrhosis, noncryptogenic, susceptibility to, | KRT8 |
| 215600 (3) | |
| Cirrhosis, North American Indian childhood | CIRH1A, NAIC, TEX292, KIAA1988 |
| type, 604901 (3) | |
| Citrullinemia, 215700 (3) | ASS |
| Citrullinemia, adult-onset type II, 603471 (3) | SLC25A13, CTLN2 |
| Citrullinemia, type II, neonatal-onset, | SLC25A13, CTLN2 |
| 605814 (3) | |
| Cleft lip/palate ectodermal dysplasia | HVEC, PVRL1, PVRR1, PRR1 |
| syndrome, 225000 (3) | |
| Cleft lip/palate, nonsyndromic, 608874 (3) | MSX1, HOX7, HYD1, OFC5 |
| Cleft palate with ankyloglossia, 303400 (3) | TBX22, CPX |
| Cleidocranial dysplasia, 119600 (3) | RUNX2, CBFA1, PEBP2A1, AML3 |
| Coats disease, 300216 (3) | NDP, ND |
| Cockayne syndrome, type A, 216400 (3) | ERCC8, CKN1, CSA |
| Cockayne syndrome, type B, 133540 (3) | ERCC6, CKN2, COFS, CSB |
| Codeine sensitivity (3) | CYP2D@, CYP2D, P450C2D |
| Coffin-Lowry syndrome, 303600 (3) | RPS6KA3, RSK2, MRX19 |
| Cohen syndrome, 216550 (3) | COH1 |
| Colchicine resistance (3) | ABCB1, PGY1, MDR1 |
| Cold-induced autoinflammatory syndrome, | CIAS1, C1orf7, FCU, FCAS |
| familial, 120100 (3) | |
| Cold-induced sweating syndrome, 272430 | CRLF1, CISS |
| (3) | |
| Coloboma, ocular, 120200 (3) | PAX6, AN2, MGDA |
| Coloboma, ocular, 120200 (3) | SHH, HPE3, HLP3, SMMCI |
| Colon adenocarcinoma (3) | RAD54B |
| Colon adenocarcinoma (3) | RAD54L, HR54, HRAD54 |
| Colon cancer (3) | BCL10 |
| Colon cancer (3) | PTPN12, PTPG1 |
| Colon cancer (3) | TGFBR2, HNPCC6 |
| Colon cancer, advanced (3) | SRC, ASV, SRC1 |
| Colon cancer, hereditary nonpolypopsis, | MLH3, HNPCC7 |
| type 7 (3) | |
| Colon cancer, somatic, 114500 (3) | PTPRJ, DEP1 |
| Colonic adenoma recurrence, reduced risk | ODC1 |
| of, 114500 (3) | |
| Colonic aganglionosis, total, with small | RET, MEN2A |
| bowel involvement (3) | |
| Colorblindness, deutan (3) | OPN1MW, GCP, CBD, CBBM |
| Colorblindness, protan (3) | OPN1LW, RCP, CBP, CBBM |
| Colorblindness, tritan (3) | OPN1SW, BCP, CBT |
| Colorectal adenomatous polyposis, | MUTYH |
| autosomal recessive, with pilomatricomas, | |
| 132600 (3) | |
| Colorectal cancer, 114500 (3) | AXIN2 |
| Colorectal cancer, 114500 (3) | BUB1B, BUBR1 |
| Colorectal cancer, 114500 (3) | EP300 |
| Colorectal cancer, 114500 (3) | PDGFRL, PDGRL, PRLTS |
| Colorectal cancer, 114500 (3) | PIK3CA |
| Colorectal cancer, 114500 (3) | TP53, P53, LFS1 |
| Colorectal cancer (3) | APC, GS, FPC |
| Colorectal cancer (3) | BAX |
| Colorectal cancer (3) | CTNNB1 |
| Colorectal cancer (3) | DCC |
| Colorectal cancer (3) | MCC |
| Colorectal cancer (3) | NRAS |
| Colorectal cancer, hereditary nonpolyposis, | MSH2, COCA1, FCC1, HNPCC1 |
| type 1, 120435 (3) | |
| Colorectal cancer, hereditary nonpolyposis, | MLH1, COCA2, HNPCC2 |
| type 2, 609310 (3) | |
| Colorectal cancer, hereditary nonpolyposis, | PMS1, PMSL1, HNPCC3 |
| type 3 (3) | |
| Colorectal cancer, hereditary nonpolyposis, | PMS2, PMSL2, HNPCC4 |
| type 4 (3) | |
| Colorectal cancer, hereditary nonpolyposis, | MSH6, GTBP, HNPCC5 |
| type 5 (3) | |
| Colorectal cancer, hereditary nonpolyposis, | TGFBR2, HNPCC6 |
| type 6 (3) | |
| Colorectal cancer, somatic, 109800 (3) | FGFR3, ACH |
| Colorectal cancer, somatic, 114500 (3) | FLCN, BHD |
| Colorectal cancer, somatic, 114500 (3) | MLH3, HNPCC7 |
| Colorectal cancer, somatic (3) | BRAF |
| Colorectal cancer, somatic (3) | DLC1 |
| Colorectal cancer, sporadic, 114500 (3) | PLA2G2A, PLA2B, PLA2L, MOM1 |
| Colorectal cancer, susceptibility to (3) | CCND1, PRAD1, BCL1 |
| Colorectal cancer with chromosomal | BUB1 |
| instability (3) | |
| Combined C6/C7 deficiency (3) | C6 |
| Combined factor V and VIII deficiency, | LMAN1, ERGIC53, F5F8D, MCFD1 |
| 227300 (3) | |
| Combined hyperlipemia, familial (3) | LPL, LIPD |
| Combined immunodeficiency, X-linked, | IL2RG, SCIDX1, SCIDX, IMD4 |
| moderate, 312863 (3) | |
| Combined oxidative phosphorylation | GFM1, EFG1, GFM |
| deficiency, 609060 (3) | |
| Combined SAP deficiency (3) | PSAP, SAP1 |
| Complex I, mitochondrial respiratory chain, | NDUFS6 |
| deficiency of, 252010 (3) | |
| Complex V, mitochondrial respiratory chain, | ATPAF2, ATP12 |
| deficiency of, 604273 (3) | |
| Cone dystrophy-1, 304020 (3) | RPGR, RP3, CRD, RP15, COD1 |
| Cone dystrophy-3, 602093 (3) | GUCA1A, GCAP |
| Cone-rod dystrophy, 300029 (3) | RPGR, RP3, CRD, RP15, COD1 |
| Cone-rod dystrophy 3 (3) | ABCA4, ABCR, STGD1, FFM, RP19 |
| Cone-rod dystrophy (3) | AIPL1, LCA4 |
| Cone-rod dystrophy 6, 601777 (3) | GUCY2D, GUC2D, LCA1, CORD6 |
| Cone-rod dystrophy 9, 608194 (3) | RPGRIP1, LCA6, CORD9 |
| Cone-rod retinal dystrophy-2, 120970 (3) | CRX, CORD2, CRD |
| Congenital bilateral absence of vas | CFTR, ABCC7, CF, MRP7 |
| deferens, 277180 (3) | |
| Congenital cataracts, facial dysmorphism, | CTDP1, FCP1, CCFDN |
| and neuropathy, 604168 (3) | |
| Congenital disorder of glycosylation, type Ic, | ALG6 |
| 603147 (3) | |
| Congenital disorder of glycosylation, type Id, | ALG3, NOT56L, CDGS4 |
| 601110 (3) | |
| Congenital disorder of glycosylation, type Ie, | DPMI, MPDS, CDGIE |
| 608799 (3) | |
| Congenital disorder of glycosylation, type If, | MPDU1, SL15, CDGIF |
| 609180 (3) | |
| Congenital disorder of glycosylation, type Ig, | ALG12 |
| 607143 (3) | |
| Congenital disorder of glycosylation, type Ih, | ALG8 |
| 608104 (3) | |
| Congenital disorder of glycosylation, type Ii, | ALG2, CDGII |
| 607906 (3) | |
| Congenital disorder of glycosylation, type II, | DIBD1, ALG9 |
| 608776 (3) | |
| Congenital disorder of glycosylation, type | SLC35C1, FUCT1 |
| IIc, 266265 (3) | |
| Congenital disorder of glycosylation, type | B4GALT1, GGTB2, GT1, GTB |
| IId, 607091 (3) | |
| Congenital disorder of glycosylation, type | COG7, CDG2E |
| IIe, 608779 (3) | |
| Congenital disorder of glycosylation, type Ij, | DPAGT2, DGPT |
| 608093 (3) | |
| Congenital disorder of glycosylation, type Ik, | ALG1, HMAT1, HMT1 |
| 608540 (3) | |
| Congestive heart failure, susceptibility to (3) | ADRA2C, ADRA2L2 |
| Congestive heart failure, susceptibility to (3) | ADRB1, ADRB1R, RHR |
| Conjunctivitis, ligneous, 217090 (3) | PLG |
| Conotruncal anomaly face syndrome, | TBX1, DGS, CTHM, CAPS, TGA, |
| 217095 (3) | DORV, VCFS, DGCR |
| Contractural arachnodactyly, congenital (3) | FBN2, CCA |
| Convulsions, familial febrile, 4, 604352 (3) | MASS1, VLGR1, KIAA0686, FEB4, |
| | USH2C |
| COPD, rate of decline of lung function in, | MMP1, CLG |
| 606963 (3) | |
| Coproporphyria (3) | CPO |
| Corneal clouding, autosomal recessive (3) | APOA1 |
| Corneal dystrophy, Avellino type, 607541 | TGFBI, CSD2, CDGG1, CSD, BIGH3, |
| (3) | CDG2 |
| Corneal dystrophy, gelatinous drop-like, | TACSTD2, TROP2, M1S1 |
| 204870 (3) | |
| Corneal dystrophy, Groenouw type I, | TGFBI, CSD2, CDGG1, CSD, BIGH3, |
| 121900 (3) | CDG2 |
| Corneal dystrophy, hereditary polymorphous | VSX1, RINX, PPCD, PPD, KTCN |
| posterior, 122000 (3) | |
| Corneal dystrophy, hereditary polymorphous | COL8A2, FECD, PPCD2 |
| posterior, 2, 122000 (3) | |
| Corneal dystrophy, lattice type I, 122200 (3) | TGFBI, CSD2, CDGG1, CSD, BIGH3, |
| | CDG2 |
| Corneal dystrophy, lattice type IIIA, 608471 | TGFBI, CSD2, CDGG1, CSD, BIGH3, |
| (3) | CDG2 |
| Corneal dystrophy, Reis-Bucklers type, | TGFBI, CSD2, CDGG1, CSD, BIGH3, |
| 608470 (3) | CDG2 |
| Corneal dystrophy, Thiel-Behnke type, | TGFBI, CSD2, CDGG1, CSD, BIGH3, |
| 602082 (3) | CDG2 |
| Corneal fleck dystrophy, 121850 (3) | PIP5K3, CFD |
| Cornea plana congenita, recessive, 217300 | KERA, CNA2 |
| (3) | |
| Cornelia de Lange syndrome, 122470 (3) | NIPBL, CDLS |
| Coronary artery disease, autosomal | MEF2A, ADCAD1 |
| dominant, 1, 608320 (3) | |
| Coronary artery disease in familial | ABCA1, ABC1, HDLDT1, TGD |
| hypercholesterolemia, protection against, | |
| 143890 (3) | |
| Coronary artery disease, susceptibility to (3) | KL |
| Coronary artery disease, susceptibility to (3) | PON1, PON, ESA |
| Coronary artery disease, susceptibility to (3) | PON2 |
| Coronary artery spasm, susceptibility to (3) | PON1, PON, ESA |
| Coronary heart disease, susceptibility to (3) | MMP3, STMY1 |
| Coronary spasms, susceptibility to (3) | NOS3 |
| Corpus callosum, agenesis of, with mental | IGBP1 |
| retardation, ocular coloboma and | |
| micrognathia, 300472 (3) | |
| Cortisol resistance (3) | NR3C1, GCR, GRL |
| Cortisone reductase deficiency, 604931 (3) | GDH |
| Cortisone reductase deficiency, 604931 (3) | HSD11B1, HSD11, HSD11L |
| Costello syndrome, 218040 (3) | HRAS |
| Coumarin resistance, 122700 (3) | CYP2A6, CYP2A3, CYP2A, P450C2A |
| Cowden disease, 158350 (3) | PTEN, MMAC1 |
| Cowden-like syndrome, 158350 (3) | BMPR1A, ACVRLK3, ALK3 |
| CPT deficiency, hepatic, type IA, 255120 (3) | CPT1A |
| CPT deficiency, hepatic, type II, 600649 (3) | CPT2 |
| CPT II deficiency, lethal neonatal, 608836 | CPT2 |
| (3) | |
| Cramps, familial, potassium-aggravated (3) | SCN4A, HYPP, NAC1A |
| Craniofacial anomalies, empty sella turcica, | VSX1, RINX, PPCD, PPD, KTCN |
| corneal endothelial changes, and abnormal | |
| retinal and auditory bipolar cells (3) | |
| Craniofacial-deafness-hand syndrome, | PAX3, WS1, HUP2, CDHS |
| 122880 (3) | |
| Craniofacial-skeletal-dermatologic dysplasia | FGFR2, BEK, CFD1, JWS |
| (3) | |
| Craniofrontonasal dysplasia, 304110 (3) | EFNB1, EPLG2, CFNS, CFND |
| Craniometaphyseal dysplasia, 123000 (3) | ANKH, HANK, ANK, CMDJ, CCAL2, |
| | CPPDD |
| Craniosynostosis, nonspecific (3) | FGFR2, BEK, CFD1, JWS |
| Craniosynostosis, type 2, 604757 (3) | MSX2, CRS2, HOX8 |
| CRASH syndrome, 303350 (3) | L1CAM, CAML1, HSAS1 |
| Creatine deficiency syndrome, X-linked, | SLC6A8, CRTR |
| 300352 (3) | |
| Creatine phosphokinase, elevated serum, | CAV3, LGMD1C |
| 123320 (3) | |
| Creatine phosphokinase, elevated serum, | CAV3, LGMD1C |
| 123320 (3) | |
| Creutzfeldt-Jakob disease, 123400 (3) | PRNP, PRIP |
| Creutzfeldt-Jakob disease, variant, | HLA-DQB1 |
| resistance to, 123400 (3) | |
| Crigler-Najjar syndrome, type I, 218800 (3) | UGT1A1, UGT1, GNT1 |
| Crigler-Najjar syndrome, type II, 606785 (3) | UGT1A1, UGT1, GNT1 |
| Crohn disease, susceptibility to, 266600 (3) | CARD15, NOD2, IBD1, CD, ACUG, |
| | PSORAS1 |
| Crohn disease, susceptibility to, 266600 (3) | DLG5, PDLG, KIAA0583 |
| Crouzon syndrome, 123500 (3) | FGFR2, BEK, CFD1, JWS |
| Crouzon syndrome with acanthosis | FGFR3, ACH |
| nigricans (3) | |
| Cryptorchidism, bilateral, 219050 (3) | LGR8, GREAT |
| Cryptorchidism, idiopathic, 219050 (3) | INSL3 |
| Currarino syndrome, 176450 (3) | HLXB9, HOXHB9, SCRA1 |
| Cutis laxa, AD, 123700 (3) | ELN |
| Cutis laxa, autosomal dominant, 123700 (3) | FBLN5, ARMD3 |
| Cutis laxa, autosomal recessive, 219100 (3) | FBLN5, ARMD3 |
| Cutis laxa, neonatal (3) | ATP7A, MNK, MK, OHS |
| Cyclic ichthyosis with epidermolytic | KRT1 |
| hyperkeratosis, 607602 (3) | |
| Cylindromatosis, familial, 132700 (3) | CYLD1, CDMT, EAC |
| Cystathioninuria, 219500 (3) | CTH |
| Cystic fibrosis, 219700 (3) | CFTR, ABCC7, CF, MRP7 |
| Cystinosis, atypical nephropathic (3) | CTNS |
| Cystinosis, late-onset juvenile or adolescent | CTNS |
| nephropathic, 219900 (3) | |
| Cystinosis, nephropathic, 219800 (3) | CTNS |
| Cystinosis, ocular nonnephropathic, 219750 | CTNS |
| (3) | |
| Cystinuria, 220100 (3) | SLC3A1, ATR1, D2H, NBAT |
| Cystinuria, type II (3) | SLC7A9, CSNU3 |
| Cystinuria, type III (3) | SLC7A9, CSNU3 |
| D-2-hydroxyglutaric aciduria, 600721 (3) | D2HGD |
| Darier disease, 124200 (3) | ATP2A2, ATP2B, DAR |
| D-bifunctional protein deficiency, 261515 (3) | HSD17B4 |
| Deafness, autosomal dominant 10, 601316 | EYA4, DFNA10, CMD1J |
| (3) | |
| Deafness, autosomal dominant 1, 124900 | DIAPH1, DFNA1, LFHL1 |
| (3) | |
| Deafness, autosomal dominant 11, | MYO7A, USH1B, DFNB2, DFNA11 |
| neurosensory, 601317 (3) | |
| Deafness, autosomal dominant 12, 601842 | TECTA, DFNA8, DFNA12, DFNB21 |
| (3) | |
| Deafness, autosomal dominant 13, 601868 | COL11A2, STL3, DFNA13 |
| (3) | |
| Deafness, autosomal dominant 15, 602459 | POU4F3, BRN3C |
| (3) | |
| Deafness, autosomal dominant 17, 603622 | MYH9, MHA, FTNS, DFNA17 |
| (3) | |
| Deafness, autosomal dominant 20/26, | ACTG1, DFNA20, DFNA26 |
| 604717 (3) | |
| Deafness, autosomal dominant 22, 606346 | MYO6, DFNA22, DFNB37 |
| (3) | |
| Deafness, autosomal dominant 2, 600101 | GJB3, CX31, DFNA2 |
| (3) | |
| Deafness, autosomal dominant 2, 600101 | KCNQ4, DFNA2 |
| (3) | |
| Deafness, autosomal dominant 28, 608641 | TFCP2L3, DFNA28 |
| (3) | |
| Deafness, autosomal dominant 3, 601544 | GJB2, CX26, DFNB1, PPK, DFNA3, |
| (3) | KID, HID |
| Deafness, autosomal dominant 3, 601544 | GJB6, CX30, DFNA3, HED, ED2 |
| (3) | |
| Deafness, autosomal dominant 36, 606705 | TMC1, DFNB7, DFNB11, DFNA36 |
| (3) | |
| Deafness, autosomal dominant 36, with | DSPP, DPP, DGI1, DFNA39, DTDP2 |
| dentinogenesis, 605594 (3) | |
| Deafness, autosomal dominant 40 (3) | CRYM, DFNA40 |
| Deafness, autosomal dominant 4, 600652 | MYH14, KIAA2034, DFNA4 |
| (3) | |
| Deafness, autosomal dominant 5 (3) | DFNA5 |
| Deafness, autosomal dominant 8, 601543 | TECTA, DFNA8, DFNA12, DFNB21 |
| (3) | |
| Deafness, autosomal dominant 9, 601369 | COCH, DFNA9 |
| (3) | |
| Deafness, autosomal dominant | MYO1A |
| nonsyndromic sensorineural, 607841 (3) | |
| Deafness, autosomal dominant, with | GJB3, CX31, DFNA2 |
| peripheral neuropathy (3) | |
| Deafness, autosomal recessive 10, | TMPRSS3, ECHOS1, DFNB8, DFNB10 |
| congenital, 605316 (3) | |
| Deafness, autosomal recessive 1, 220290 | GJB2, CX26, DFNB1, PPK, DFNA3, |
| (3) | KID, HID |
| Deafness, autosomal recessive 12, 601386 | CDH23, USH1D |
| (3) | |
| Deafness, autosomal recessive 12, modifier | ATP2B2, PMCA2 |
| of, 601386 (3) | |
| Deafness, autosomal recessive 16, 603720 | STRC, DFNB16 |
| (3) | |
| Deafness, autosomal recessive 18, 602092 | USH1C, DFNB18 |
| (3) | |
| Deafness, autosomal recessive 21, 603629 | TECTA, DFNA8, DFNA12, DFNB21 |
| (3) | |
| Deafness, autosomal recessive 22, 607039 | OTOA, DFNB22 |
| (3) | |
| Deafness, autosomal recessive 23, 609533 | PCDH15, DFNB23 |
| (3) | |
| Deafness, autosomal recessive 29 (3) | CLDN14, DFNB29 |
| Deafness, autosomal recessive 2, | MYO7A, USH1B, DFNB2, DFNA11 |
| neurosensory, 600060 (3) | |
| Deafness, autosomal recessive 30, 607101 | MYO3A, DFNB30 |
| (3) | |
| Deafness, autosomal recessive 31, 607084 | WHRN, CIP98, KIAA1526, DFNB31 |
| (3) | |
| Deafness, autosomal recessive 3, 600316 | MYO15A, DFNB3 |
| (3) | |
| Deafness, autosomal recessive 36, 609006 | ESPN |
| (3) | |
| Deafness, autosomal recessive 37, 607821 | MYO6, DFNA22, DFNB37 |
| (3) | |
| Deafness, autosomal recessive (3) | GJB3, CX31, DFNA2 |
| Deafness, autosomal recessive 4, 600791 | SLC26A4, PDS, DFNB4 |
| (3) | |
| Deafness, autosomal recessive 61 (3) | PRES, DFNB61, SLC26A5 |
| Deafness, autosomal recessive 6, 600971 | TMIE, DFNB6 |
| (3) | |
| Deafness, autosomal recessive 7, 600974 | TMC1, DFNB7, DFNB11, DFNA36 |
| (3) | |
| Deafness, autosomal recessive 8, childhood | TMPRSS3, ECHOS1, DFNB8, DFNB10 |
| onset, 601072 (3) | |
| Deafness, autosomal recessive 9, 601071 | OTOF, DFNB9, NSRD9 |
| (3) | |
| Deafness, congenital heart defects, and | JAG1, AGS, AHD |
| posterior embryotoxon (3) | |
| Deafness, nonsyndromic (3) ( ) | KIAA1199 |
| Deafness, nonsyndromic neurosensory, | GJB6, CX30, DFNA3, HED, ED2 |
| digenic (3) | |
| Deafness, sensorineural, with hypertrophic | MYO6, DFNA22, DFNB37 |
| cardiomyopathy, 606346 (3) | |
| Deafness, X-linked 1, progressive (3) | TIMM8A, DFN1, DDP, MTS, DDP1 |
| Deafness, X-linked 3, conductive, with | POU3F4, DFN3 |
| stapes fixation, 304400 (3) | |
| Debrisoquine sensitivity (3) | CYP2D@, CYP2D, P450C2D |
| Dejerine-Sottas disease, 145900 (3) | PMP22, CMT1A, CMT1E, DSS |
| Dejerine-Sottas neuropathy, 145900 (3) | EGR2, KROX20 |
| Dejerine-Sottas neuropathy, autosomal | PRX, CMT4F |
| recessive, 145900 (3) | |
| Dejerine-Sottas syndrome, 145900 (3) | MPZ, CMT1B, CMTDI3, CHM, DSS |
| Delayed sleep phase syndrome, | AANAT, SNAT |
| susceptibility to (3) | |
| Dementia, familial British, 176500 (3) | ITM2B, BRI, ABRI, FBD |
| Dementia, familial Danish, 117300 (3) | ITM2B, BRI, ABRI, FBD |
| Dementia, frontotemporal, 600274 (3) | PSEN1, AD3 |
| Dementia, frontotemporal, with | MAPI, MTBT1, DDPAC, MSTD |
| parkinsonism, 600274 (3) | |
| Dementia, Lewy body, 127750 (3) | SNCA, NACP, PARK1, PARK4 |
| Dementia, Lewy body, 127750 (3) | SNCB |
| Dementia, Pick disease-like, 172700 (3) | MAPI, MTBT1, DDPAC, MSTD |
| Dementia, vascular, susceptibility to (3) | TNF, TNFA |
| Dengue fever, protection against (3) | CD209, CDSIGN |
| Dental anomalies, isolated (3) | RUNX2, CBFA1, PEBP2A1, AML3 |
| Dentatorubro-pallidoluysian atrophy, 125370 | DRPLA |
| (3) | |
| Dent disease, 300009 (3) | CLCN5, CLCK2, NPHL2, DENTS |
| Dentin dysplasia, type II, 125420 (3) | DSPP, DPP, DGI1, DFNA39, DTDP2 |
| Dentinogenesis imperfecta, Shields type II, | DSPP, DPP, DGI1, DFNA39, DTDP2 |
| 125490 (3) | |
| Dentinogenesis imperfecta, Shields type III, | DSPP, DPP, DGI1, DFNA39, DTDP2 |
| 125500 (3) | |
| Dent syndrome, 300009 (3) | OCRL, LOCR, OCRL1, NPHL2 |
| Denys-Drash syndrome, 194080 (3) | WT1 |
| Dermatofibrosarcoma protuberans (3) | PDGFB, SIS |
| De Sanctis-Cacchione syndrome, 278800 | ERCC6, CKN2, COFS, CSB |
| (3) | |
| Desmoid disease, hereditary, 135290 (3) | APC, GS, FPC |
| Desmosterolosis, 602398 (3) | DHCR24, KIAA0018 |
| Diabetes insipidus, nephrogenic, 304800 (3) | AVPR2, DIR, DI1, ADHR |
| Diabetes insipidus, nephrogenic, autosomal | AQP2 |
| dominant, 125800 (3) | |
| Diabetes insipidus, nephrogenic, autosomal | AQP2 |
| recessive, 222000 (3) | |
| Diabetes insipidus, neurohypophyseal, | AVP, AVRP, VP |
| 125700 (3) | |
| Diabetes mellitus, 125853 (3) | ABCC8, SUR, PHHI, SUR1 |
| Diabetes mellitus, insulin-dependent, | TCF1, HNF1A, MODY3 |
| 222100 (3) | |
| Diabetes mellitus, insulin-dependent, 5, | SUMO4, IDDM5 |
| 600320 (3) | |
| Diabetes mellitus, insulin-dependent, | PTPN8, PEP, PTPN22, LYP |
| susceptibility to, 222100 (3) | |
| Diabetes mellitus, insulin-resistant, with | INSR |
| acanthosis nigricans (3) | |
| Diabetes mellitus, insulin-resistant, with | PPARG, PPARG1, PPARG2 |
| acanthosis nigricans and hypertension, | |
| 604367 (3) | |
| Diabetes mellitus, neonatal-onset, 606176 | GCK |
| (3) | |
| Diabetes mellitus, noninsulin-dependent, | GCGR |
| 125853 (3) | |
| Diabetes mellitus, noninsulin-dependent, | GPD2 |
| 125853 (3) | |
| Diabetes mellitus, noninsulin-dependent, | HNF4A, TCF14, MODY1 |
| 125853 (3) | |
| Diabetes mellitus, noninsulin-dependent, | IRS2 |
| 125853 (3) | |
| Diabetes mellitus, noninsulin-dependent, | MAPK8IP1, IB1 |
| 125853 (3) | |
| Diabetes mellitus, noninsulin-dependent, | NEUROD1, NIDDM |
| 125853 (3) | |
| Diabetes mellitus, noninsulin-dependent, | TCF2, HNF2 |
| 125853 (3) | |
| Diabetes mellitus, noninsulin-dependent, 2, | TCF1, HNF1A, MODY3 |
| 125853 (3) | |
| Diabetes mellitus, noninsulin-dependent (3) | IRS1 |
| Diabetes mellitus, noninsulin-dependent (3) | SLC2A2, GLUT2 |
| Diabetes mellitus, noninsulin-dependent (3) | SLC2A4, GLUT4 |
| Diabetes mellitus, noninsulin-dependent, | CAPN10 |
| 601283 (3) | |
| Diabetes mellitus, non-insulin-dependent, | ENPP1, PDNP1, NPPS, M6S1, PCA1 |
| susceptibility to, 125853 (3) | |
| Diabetes mellitus, noninsulin-dependent, | RETN, RSTN, FIZZ3 |
| susceptibility to, 125853 (3) | |
| Diabetes mellitus, permanent neonatal, with | PTF1A |
| cerebellar agenesis, 609069 (3) | |
| Diabetes mellitus, permanent neonatal, with | KCNJ11, BIR, PHHI |
| neurologic features, 606176 (3) | |
| Diabetes mellitus, type II, 125853 (3) | AKT2 |
| Diabetes mellitus, type II, susceptibility to, | IPF1 |
| 125853 (3) | |
| Diabetes mellitus, type I, susceptibility to, | FOXP3, IPEX, AIID, XPID, PIDX |
| 222100 (3) | |
| Diabetes, permanent neonatal, 606176 (3) | KCNJ11, BIR, PHHI |
| Diabetic nephropathy, susceptibility to, | ACE, DCP1, ACE1 |
| 603933 (3) | |
| Diabetic retinopathy, NIDDM-related, | VEGF |
| susceptibility to, 125853 (3) | |
| Diastrophic dysplasia, 222600 (3) | SLC26A2, DTD, DTDST, D5S1708, |
| | EDM4 |
| Diastrophic dysplasia, broad bone- | SLC26A2, DTD, DTDST, D5S1708, |
| platyspondylic variant (3) | EDM4 |
| DiGeorge syndrome, 188400 (3) | TBX1, DGS, CTHM, CAFS, TGA, |
| | DORV, VCFS, DGCR |
| Dihydropyrimidinuria (3) | DPYS, DHP |
| Dilated cardiomyopathy with woolly hair and | DSP, KPPS2, PPKS2 |
| keratoderma, 605676 (3) | |
| Dimethylglycine dehydrogenase deficiency, | DMGDH, DMGDHD |
| 605850 (3) | |
| Disordered steroidogenesis, isolated (3) | POR |
| Dissection of cervical arteries (3) | COL1A1 |
| DNA ligase I deficiency (3) | LIG1 |
| DNA topoisomerase I, camptothecin- | TOP1 |
| resistant (3) | |
| DNA topoisomerase II, resistance to | TOP2A, TOP2 |
| inhibition of, by amsacrine (3) | |
| Dopamine-beta-hydroxylase activity levels, | DBH |
| plasma (3) | |
| Dopamine beta-hydroxylase deficiency, | DBH |
| 223360 (3) | |
| Dosage-sensitive sex reversal, 300018 (3) | DAX1, AHC, AHX, NROB1 |
| Double-outlet right ventricle, 217095 (3) | CFC1, CRYPTIC, HTX2 |
| Down syndrome, risk of, 190685 (3) | MTR |
| Doyne honeycomb degeneration of retina, | EFEMP1, FBNL, DHRD |
| 126600 (3) | |
| Drug addiction, susceptibility to (3) | FAAH |
| Duane-radial ray syndrome, 607323 (3) | SALL4, HSAL4 |
| Dubin-Johnson syndrome, 237500 (3) | ABCC2, CMOAT |
| Duchenne muscular dystrophy, 310200 (3) | DMD, BMD |
| Dyggve-Melchior-Clausen disease, 223800 | DYM, FLJ90130, DMC, SMC |
| (3) | |
| Dysalbuminemic hyperthyroxinemia (3) | ALB |
| Dysautonomia, familial, 223900 (3) | IKBKAP, IKAP |
| Dyschromatosis symmetrica hereditaria, | ADAR, DRADA, DSH, DSRAD |
| 127400 (3) | |
| Dyserythropoietic anemia with | GATA1, GF1, ERYF1, NFE1 |
| thrombocytopenia, 300367 (3) | |
| Dysfibrinogenemia, alpha type, causing | FGA |
| bleeding diathesis (3) | |
| Dysfibrinogenemia, alpha type, causing | FGA |
| recurrent thrombosis (3) | |
| Dysfibrinogenemia, beta type (3) | FGB |
| Dysfibrinogenemia, gamma type (3) | FGG |
| Dyskeratosis congenita-1, 305000 (3) | DKC1, DKC |
| Dyskeratosis congenita, autosomal | TERC, TRC3, TR |
| dominant, 127550 (3) | |
| Dyslexia, susceptibility to, 1, 127700 (3) | DYX1C1, DYXC1, DYX1 |
| Dyslexia, susceptibility to, 2, 600202 (3) | KIAA0319, DYX2, DYLX2, DLX2 |
| Dysprothrombinemia (3) | F2 |
| Dyssegmental dysplasia, Silverman- | HSPG2, PLC, SJS, SJA, SJS1 |
| Handmaker type, 224410 (3) | |
| Dystonia-12, 128235 (3) | ATP1A3, DYT12, RDP |
| Dystonia-1, torsion, 128100 (3) | DYT1, TOR1A |
| Dystonia, DOPA-responsive, 128230 (3) | GCH1, DYT5 |
| Dystonia, early-onset atypical, with | DYT1, TOR1A |
| myoclonic features (3) | |
| Dystonia, myoclonic, 159900 (3) | DRD2 |
| Dystonia, myoclonic, 159900 (3) | SGCE, DYT11 |
| Dystonia, primary cervical (3) | DRD5, DRD1B, DRD1L2 |
| Dystransthyretinemic hyperthyroxinemia(3) | TTR, PALB |
| EBD, Bart type, 132000 (3) | COL7A1 |
| EBD, localisata variant (3) | COL7A1 |
| Ectodermal dysplasia-1, anhidrotic, 305100 | ED1, EDA, HED |
| (3) | |
| Ectodermal dysplasia 2, hidrotic, 129500 (3) | GJB6, CX30, DFNA3, HED, ED2 |
| Ectodermal dysplasia, anhidrotic, 224900 | EDARADD |
| (3) | |
| Ectodermal dysplasia, anhidrotic, | IKBKG, NEMO, FIP3, IP2 |
| lymphedema and immunodeficiency, 300301 | |
| (3) | |
| Ectodermal dysplasia, anhidrotic, with T-cell | NFKBIA, IKBA |
| immunodeficiency (3) | |
| Ectodermal dysplasia, hypohidrotic, | EDAR, DL, ED3, EDA3 |
| autosomal dominant, 129490 (3) | |
| Ectodermal dysplasia, hypohidrotic, | EDAR, DL, ED3, EDA3 |
| autosomal recessive, 224900 (3) | |
| Ectodermal dysplasia, hypohidrotic, with | IKBKG, NEMO, FIP3, IP2 |
| immune deficiency, 300291 (3) | |
| Ectodermal dysplasia, Margarita Island type, | HVEC, PVRL1, PVRR1, PRR1 |
| 225060 (3) | |
| Ectodermal dysplasia/skin fragility | PKP1 |
| syndrome, 604536 (3) | |
| Ectopia lentis, familial, 129600 (3) | FBN1, MFS1, WMS |
| Ectopia pupillae, 129750 (3) | PAX6, AN2, MGDA |
| Ectrodactyly, ectodermal dysplasia, and | TP73L, TP63, KET, EEC3, SHFM4, |
| cleft lip/palate syndrome 3, 604292 (3) | LMS, RHS |
| Ehlers-Danlos due to tenascin X deficiency, | TNXB, TNX, TNXB1, TNXBS, TNXB2 |
| 606408 (3) | |
| Ehlers-Danlos syndrome, hypermobility | TNXB, TNX, TNXB1, TNXBS, TNXB2 |
| type, 130020 (3) | |
| Ehlers-Danlos syndrome, progeroid form, | B4GALT7, XGALT1, XGPT1 |
| 130070 (3) | |
| Ehlers-Danlos syndrome, type I, 130000 (3) | COL1A1 |
| Ehlers-Danlos syndrome, type I, 130000 (3) | COL5A1 |
| Ehlers-Danlos syndrome, type I, 130000 (3) | COL5A2 |
| Ehlers-Danlos syndrome, type II, 130010 (3) | COL5A1 |
| Ehlers-Danlos syndrome, type III, 130020 | COL3A1 |
| (3) | |
| Ehlers-Danlos syndrome, type IV, 130050 | COL3A1 |
| (3) | |
| Ehlers-Danlos syndrome, type VI, 225400 | PLOD, PLOD1 |
| (3) | |
| Ehlers-Danlos syndrome, type VII, 130060 | COL1A1 |
| (3) | |
| Ehlers-Danlos syndrome, type VIIA2, | COL1A2 |
| 130060 (3) | |
| Ehlers-Danlos syndrome, type VIIC, 225410 | ADAMTS2, NPI |
| (3) | |
| Elite sprint athletic performance (3) | ACTN3 |
| Elliptocytosis-1 (3) | EPB41, EL1 |
| Elliptocytosis-2 (3) | SPTA1 |
| Elliptocytosis-3 (3) | SPTB |
| Elliptocytosis, Malaysian-Melanesian type | SLC4A1, AE1, EPB3 |
| (3) | |
| Ellis-van Creveld syndrome, 225500 (3) | EVC |
| Ellis-van Creveld syndrome, 225500 (3) | LBN, EVC2 |
| Emery-Dreifuss muscular dystrophy, | EMD, EDMD, STA |
| 310300 (3) | |
| Emery-Dreifuss muscular dystrophy, AD, | LMNA, LMN1, EMD2, FPLD, CMD1A, |
| 181350 (3) | HGPS, LGMD1B |
| Emery-Dreifuss muscular dystrophy, AR, | LMNA, LMN1, EMD2, FPLD, CMD1A, |
| 604929 (3) | HGPS, LGMD1B |
| Emphysema (3) | PI, AAT |
| Emphysema-cirrhosis (3) | PI, AAT |
| Encephalopathy, familial, with neuroserpin | SERPINI1, PI12 |
| inclusion bodies, 604218 (3) | |
| Encephalopathy, progressive mitochondrial, | COX10 |
| with proximal renal tubulopathy due to | |
| cytochrome c oxidase deficiency (3) | |
| Enchondromatosis, Oilier type, 166000 (3) | PTHR1, PTHR |
| Endometrial carcinoma (3) | CDH1, UVO |
| Endometrial carcinoma (3) | MSH3 |
| Endometrial carcinoma (3) | MSH6, GTBP, HNPCC5 |
| Endometrial carcinoma (3) | PTEN, MMAC1 |
| Endotoxin hyporesponsiveness (3) | TLR4 |
| Endplate acetylcholinesterase deficiency, | COLQ, EAD |
| 603034 (3) | |
| Enhanced S-cone syndrome, 268100 (3) | NR2E3, PNR, ESCS |
| Enlarged vestibular aqueduct, 603545 (3) | SLC26A4, PDS, DFNB4 |
| Enolase-beta deficiency (3) | ENO3 |
| Enterokinase deficiency, 226200 (3) | PRSS7, ENTK |
| Eosinophil peroxidase deficiency, 261500 | EPX |
| (3) | |
| Epidermodysplasia verruciformis, 226400 | EVER1, EV1 |
| (3) | |
| Epidermodysplasia verruciformis, 226400 | EVER2, EV2 |
| (3) | |
| Epidermolysis bullosa dystrophica, AD, | COL7A1 |
| 131750 (3) | |
| Epidermolysis bullosa dystrophica, AR, | COL7A1 |
| 226600 (3) | |
| Epidermolysis bullosa, generalized atrophic | COL17A1, BPAG2 |
| benign, 226650 (3) | |
| Epidermolysis bullosa, generalized atrophic | ITGB4 |
| benign, 226650 (3) | |
| Epidermolysis bullosa, generalized atrophic | LAMA3, LOCS |
| benign, 226650 (3) | |
| Epidermolysis bullosa, generalized atrophic | LAMB3 |
| benign, 226650 (3) | |
| Epidermolysis bullosa, generalized atrophic | LAMC2, LAMNB2, LAMB2T |
| benign, 226650 (3) | |
| Epidermolysis bullosa, Herlitz junctional | LAMB3 |
| type, 226700 (3) | |
| Epidermolysis bullosa, Herlitz junctional | LAMC2, LAMNB2, LAMB2T |
| type, 226700 (3) | |
| Epidermolysis bullosa, junctional, Herlitz | LAMA3, LOCS |
| type, 226700 (3) | |
| Epidermolysis bullosa, junctional, with | ITGB4 |
| pyloric atresia, 226730 (3) | |
| Epidermolysis bullosa, junctional, with | ITGA6 |
| pyloric stenosis, 226730 (3) | |
| Epidermolysis bullosa, lethal acantholytic, | DSP, KPPS2, PPKS2 |
| 609638 (3) | |
| Epidermolysis bullosa of hands and feet, | ITGB4 |
| 131800 (3) | |
| Epidermolysis bullosa, pretibial, 131850 (3) | COL7A1 |
| Epidermolysis bullosa pruriginosa, 604129 | COL7A1 |
| (3) | |
| Epidermolysis bullosa simplex, Koebner, | KRT14 |
| Dowling-Meara, and Weber-Cockayne types, | |
| 131900, 131760, 131800 (3) | |
| Epidermolysis bullosa simplex, Koebner, | KRT5 |
| Dowling-Meara, and Weber-Cockayne types, | |
| 131900, 131760, 131800 (3) | |
| Epidermolysis bullosa simplex, Ogna type, | PLEC1, PLTN, EBS1 |
| 131950 (3) | |
| Epidermolysis bullosa simplex, recessive, | KRT14 |
| 601001 (3) | |
| Epidermolysis bullosa simplex with mottled | KRT5 |
| pigmentation, 131960 (3) | |
| Epidermolytic hyperkeratosis, 113800 (3) | KRT10 |
| Epidermolytic hyperkeratosis, 113800 (3) | KRT1 |
| Epidermolytic palmoplantar keratoderma, | KRT9, EPPK |
| 144200 (3) | |
| Epilepsy, benign, neonatal, type 1, 121200 | KCNQ2, EBN1 |
| (3) | |
| Epilepsy, benign neonatal, type 2, 121201 | KCNQ3, EBN2, BFNC2 |
| (3) | |
| Epilepsy, childhood absence, 607681 (3) | GABRG2, GEFSP3, CAE2, ECA2 |
| Epilepsy, childhood absence, 607682 (3) | CLCN2, EGMA, ECA3, EGI3 |
| Epilepsy, childhood absence, evolving to | JRK, JH8 |
| juvenile myoclonic epilepsy (3) | |
| Epilepsy, generalized idiopathic, 600669 (3) | CACNB4, EJM |
| Epilepsy, generalized, with febrile seizures | GABRG2, GEFSP3, CAE2, ECA2 |
| plus, 604233 (3) | |
| Epilepsy, generalized, with febrile seizures | SCN1A, GEFSP2, SMEI |
| plus, type 2, 604233 (3) | |
| Epilepsy, idopathic generalized, | ME2 |
| susceptibility to, 600669 (3) | |
| Epilepsy, juvenile absence, 607631 (3) | CLCN2, EGMA, ECA3, EGI3 |
| Epilepsy, juvenile myoclonic, 606904 (3) | CACNB4, EJM |
| Epilepsy, juvenile myoclonic, 606904 (3) | CLCN2, EGMA, ECA3, EGI3 |
| Epilepsy, juvenile myoclonic, 606904 (3) | GABRA1, EJM |
| Epilepsy, myoclonic, Lafora type, 254780 | EPM2A, MELF, EPM2 |
| (3) | |
| Epilepsy, myoclonic, Lafora type, 254780 | NHLRC1, EPM2A, EPM2B |
| (3) | |
| Epilepsy, neonatal myoclonic, with | SLC25A22, GC1 |
| suppression-burst pattern, 609304 (3) | |
| Epilepsy, nocturnal frontal lobe, 1, 600513 | CHRNA4, ENFL1 |
| (3) | |
| Epilepsy, nocturnal frontal lobe, 3, 605375 | CHRNB2, EFNL3 |
| (3) | |
| Epilepsy, partial, with auditory features, | LGI1, EPT, ETL1 |
| 600512 (3) | |
| Epilepsy, progressive myoclonic 1, 254800 | CSTB, STFB, EPM1 |
| (3) | |
| Epilepsy, progressive myoclonic 2B, 254780 | NHLRC1, EPM2A, EPM2B |
| (3) | |
| Epilepsy, severe myoclonic, of infancy, | SCN1A, GEFSP2, SMEI |
| 607208 (3) | |
| Epilepsy with grand mal seizures on | CLCN2, EGMA, ECA3, EGI3 |
| awakening, 607628 (3) | |
| Epilepsy, X-linked, with variable learning | SYN1 |
| disabilities and behavior disorders, 300491 | |
| (3) | |
| Epiphyseal dysplasia, multiple 1, 132400 (3) | COMP, EDM1, MED, PSACH |
| Epiphyseal dysplasia, multiple, 226900 (3) | SLC26A2, DTD, DTDST, D5S1708, |
| | EDM4 |
| Epiphyseal dysplasia, multiple, 3, 600969 | COL9A3, EDM3, IDD |
| (3) | |
| Epiphyseal dysplasia, multiple, 5, 607078 | MATN3, EDM5, HOA |
| (3) | |
| Epiphyseal dysplasia, multiple, COL9A1- | COL9A1, MED |
| related (3) | |
| Epiphyseal dysplasia, multiple, type 2, | COL9A2, EDM2 |
| 600204 (3) | |
| Epiphyseal dysplasia, multiple, with | COL9A3, EDM3, IDD |
| myopathy (3) | |
| Episodic ataxia/myokymia syndrome, | KCNA1, AEMK, EA1 |
| 160120 (3) | |
| Episodic ataxia, type 2, 108500 (3) | CACNA1A, CACNL1A4, SCA6 |
| Epithelial ovarian cancer, somatic, 604370 | OPCML |
| (3) | |
| Epstein syndrome, 153650 (3) | MYH9, MHA, FTNS, DFNA17 |
| Erythermalgia, primary, 133020 (3) | SCN9A, NENA, PN1 |
| Erythremias, alpha-(3) | HBA1 |
| Erythremias, beta-(3) | HBB |
| Erythrocytosis (3) | HBA2 |
| Erythrocytosis, familial, 133100 (3) | EPOR |
| Erythrokeratoderma, progressive symmetric, | LOR |
| 602036 (3) | |
| Erythrokeratodermia variabilis, 133200 (3) | GJB3, CX31, DFNA2 |
| Erythrokeratodermia variabilis with | GJB4, CX30.3 |
| erythema gyratum repens, 133200 (3) | |
| Esophageal cancer, 133239 (3) | TGFBR2, HNPCC6 |
| Esophageal carcinoma, somatic, 133239 (3) | RNF6 |
| Esophageal squamous cell carcinoma, | LZTS1, F37, FEZ1 |
| 133239 (3) | |
| Esophageal squamous cell carcinoma, | WWOX, FOR |
| 133239 (3) | |
| Estrogen resistance (3) | ESR1, ESR |
| Ethylmalonic encephalopathy, 602473 (3) | ETHE1, HSCO, D83198 |
| Ewing sarcoma (3) | EWSR1, EWS |
| Exertional myoglobinuria due to deficiency | LDHA, LDH1 |
| of LDH-A (3) | |
| Exostoses, multiple, type 1, 133700 (3) | EXT1 |
| Exostoses, multiple, type 2, 133701 (3) | EXT2 |
| Exudative vitreoretinopathy, 133780 (3) | FZD4, EVR1 |
| Exudative vitreoretinopathy, dominant, | LRP5, BMND1, LRP7, LR3, OPPG, |
| 133780 (3) | VBCH2 |
| Exudative vitreoretinopathy, recessive, | LRP5, BMND1, LRP7, LR3, OPPG, |
| 601813 (3) | VBCH2 |
| Exudative vitreoretinopathy, X-linked, | NDP, ND |
| 305390 (3) | |
| Eye anomalies, multiplex (3) | PAX6, AN2, MGDA |
| Ezetimibe, nonresponse to (3) | NPC1L1 |
| Fabry disease (3) | GLA |
| Facioscapulohumeral muscular dystrophy- | FSHMD1A, FSHD1A |
| 1A (3) | |
| Factor H and factor H-like 1 (3) | HF1, CFH, HUS |
| Factor V and factor VIII, combined | MCFD2 |
| deficiency of, 227300 (3) | |
| Factor VII deficiency (3) | F7 |
| Factor X deficiency (3) | F10 |
| Factor XI deficiency, autosomal dominant | F11 |
| (3) | |
| Factor XI deficiency, autosomal recessive | F11 |
| (3) | |
| Factor XII deficiency (3) | F12, HAF |
| Factor XIIIA deficiency (3) | F13A1, F13A |
| Factor XIIIB deficiency (3) | F13B |
| Familial Mediterranean fever, 249100 (3) | MEFV, MEF, FMF |
| Fanconi anemia, complementation group A, | FANCA, FACA, FA1, FA, FAA |
| 227650 (3) | |
| Fanconi anemia, complementation group B, | FAAP95, FAAP90, FLJ34064, FANCB |
| 300514 (3) | |
| Fanconi anemia, complementation group C | FANCC, FACC |
| (3) | |
| Fanconi anemia, complementation group | BRCA2, FANCD1 |
| D1, 605724 (3) | |
| Fanconi anemia, complementation group D2 | FANCD2, FANCD, FACD, FAD |
| (3) | |
| Fanconi anemia, complementation group E | FANCE, FACE |
| (3) | |
| Fanconi anemia, complementation group F | FANCF |
| (3) | |
| Fanconi anemia, complementation group G | XRCC9, FANCG |
| (3) | |
| Fanconi anemia, complementation group J, | BRIP1, BACH1, FANCJ |
| 609054 (3) | |
| Fanconi anemia, complementation group L | PHF9, FANCL |
| (3) | |
| Fanconi anemia, complementation group M | FANCM, KIAA1596 |
| (3) | |
| Fanconi-Bickel syndrome, 227810 (3) | SLC2A2, GLUT2 |
| Farber lipogranulomatosis (3) | ASAH, AC |
| Fatty liver, acute, of pregnancy (3) | HADHA, MTPA |
| Favism (3) | G6PD, G6PD1 |
| Fechtner syndrome, 153640 (3) | MYH9, MHA, FTNS, DFNA17 |
| Feingold syndrome, 164280 (3) | MYCN, NMYC, ODED, MODED |
| Fertile eunuch syndrome, 228300 (3) | GNRHR, LHRHR |
| Fibrocalculous pancreatic diabetes, | SPINK1, PSTI, PCTT, TATI |
| susceptibility to (3) | |
| Fibromatosis, gingival, 135300 (3) | SOS1, GINGF, GF1, HGF |
| Fibromatosis, juvenile hyaline, 228600 (3) | ANTXR2, CMG2, JHF, ISH |
| Fibrosis of extraocular muscles, congenital, | KIF21A, KIAA1708, FEOM1, CFEOM1 |
| 1, 135700 (3) | |
| Fibrosis of extraocular muscles, congenital, | PHOX2A, ARIX, CFEOM2 |
| 2, 602078 (3) | |
| Fibular hypoplasia and complex | GDF5, CDMP1 |
| brachydactyly, 228900 (3) | |
| Fish-eye disease, 136120 (3) | LCAT |
| Fish-odor syndrome, 602079 (3) | FMO3 |
| Fitzgerald factor deficiency (3) | KNG |
| Fluorouracil toxicity, sensitivity to (3) | DPYD, DPD |
| Focal cortical dysplasia, Taylor balloon cell | TSC1, LAM |
| type, 607341 (3) | |
| Follicle-stimulating hormone deficiency, | FSHB |
| isolated, 229070 (3) | |
| Forebrain defects (3) | TDGF1 |
| Foveal hypoplasia, isolated, 136520 (3) | PAX6, AN2, MGDA |
| Foveomacular dystrophy, adult-onset, with | RDS, RP7, PRPH2, PRPH, AVMD, |
| choroidal neovascularization, 608161 (3) | AOFMD |
| Fragile X syndrome (3) | FMR1, FRAXA |
| Fraser syndrome, 219000 (3) | FRAS1 |
| Fraser syndrome, 219000 (3) | FREM2 |
| Frasier syndrome, 136680 (3) | WT1 |
| Friedreich ataxia, 229300 (3) | FRDA, FARR |
| Friedreich ataxia with retained reflexes, | FRDA, FARR |
| 229300 (3) | |
| Frontometaphyseal dysplasia, 304120 (3) | FLNA, FLN1, ABPX, NHBP, OPD1, |
| | OPD2, FMD, MNS |
| Fructose-bisphosphatase deficiency (3) | FBP1 |
| Fructose intolerance (3) | ALDOB |
| Fructosuria (3) | KHK |
| Fuchs endothelial corneal dystrophy, | COL8A2, FECD, PPCD2 |
| 136800 (3) | |
| Fucosidosis (3) | FUCA1 |
| Fucosyltransferase-6 deficiency (3) | FUT6 |
| Fumarase deficiency, 606812 (3) | FH |
| Fundus albipunctatus, 136880 (3) | RDH5 |
| Fundus albipunctatus, 136880 (3) | RLBP1 |
| Fundus flavimaculatus, 248200 (3) | ABCA4, ABCR, STGD1, FFM, RP19 |
| G6PD deficiency (3) | G6PD, G6PD1 |
| GABA-transaminase deficiency (3) | ABAT, GABAT |
| Galactokinase deficiency with cataracts, | GALK1 |
| 230200 (3) | |
| Galactose epimerase deficiency, 230350 (3) | GALE |
| Galactosemia, 230400 (3) | GALT |
| Galactosialidosis (3) | PPGB, GSL, NGBE, GLB2, CTSA |
| GAMT deficiency (3) | GAMT |
| Gardner syndrome (3) | APC, GS, FPC |
| Gastric cancer, 137215 (3) | APC, GS, FPC |
| Gastric cancer, 137215 (3) | IRF1, MAR |
| Gastric cancer, familial diffuse, 137215 (3) | CDH1, UVO |
| Gastric cancer risk after H. pylori infection, | IL1B |
| 137215 (3) | |
| Gastric cancer risk after H. pylori infection, | IL1RN |
| 137215 (3) | |
| Gastric cancer, somatic, 137215 (3) | CASP10, MCH4, ALPS2 |
| Gastric cancer, somatic, 137215 (3) | ERBB2, NGL, NEU, HER2 |
| Gastric cancer, somatic, 137215 (3) | FGFR2, BEK, CFD1, JWS |
| Gastric cancer, somatic, 137215 (3) | KLF6, COPEB, BCD1, ZF9 |
| Gastric cancer, somatic, 137215 (3) | MUTYH |
| Gastrointestinal stromal tumor, somatic, | KIT, PBT |
| 606764 (3) | |
| Gastrointestinal stromal tumor, somatic, | PDGFRA |
| 606764 (3) | |
| Gaucher disease, 230800 (3) | GBA |
| Gaucher disease, variant form (3) | PSAP, SAP1 |
| Gaucher disease with cardiovascular | GBA |
| calcification, 231005 (3) | |
| Gaze palsy, horizontal, with progressive | ROBO3, RBIG1, RIG1, HGPPS |
| scoliosis, 607313 (3) | |
| Generalized epilepsy and paroxysmal | KCNMA1, SLO |
| dyskinesia, 609446 (3) | |
| Generalized epilepsy with febrile seizures | SCN1B, GEFSP1 |
| plus, 604233 (3) | |
| Germ cell tumor (3) | BCL10 |
| Germ cell tumors, 273300 (3) | KIT, PBT |
| Gerstmann-Straussler disease, 137440 (3) | PRNP, PRIP |
| Giant axonal neuropathy-1, 256850 (3) | GAN, GAN1 |
| Giant-cell fibroblastoma (3) | PDGFB, SIS |
| Giant cell hepatitis, neonatal, 231100 (3) | CYP7B1 |
| Giant platelet disorder, isolated (3) | GP1BB |
| Gilbert syndrome, 143500 (3) | UGT1A1, UGT1, GNT1 |
| Gitelman syndrome, 263800 (3) | SLC12A3, NCCT, TSC |
| Glanzmann thrombasthenia, type A, 273800 | ITGA2B, GP2B, CD41B |
| (3) | |
| Glanzmann thrombasthenia, type B (3) | ITGB3, GP3A |
| Glaucoma 1A, primary open angle, juvenile- | MYOC, TIGR, GLC1A, JOAG, GPOA |
| onset, 137750 (3) | |
| Glaucoma 1A, primary open angle, | MYOC, TIGR, GLC1A, JOAG, GPOA |
| recessive (3) | |
| Glaucoma 1E, primary open angle, adult- | OPTN, GLC1E, FIP2, HYPL, NRP |
| onset, 137760 (3) | |
| Glaucoma 3A, primary congenital, 231300 | CYP1B1, GLC3A |
| (3) | |
| Glaucoma, early-onset, digenic (3) | CYP1B1, GLC3A |
| Glaucoma, early-onset, digenic (3) | MYOC, TIGR, GLC1A, JOAG, GPOA |
| Glaucoma, normal tension, susceptibility to, | OPA1, NTG, NPG |
| 606657 (3) | |
| Glaucoma, normal tension, susceptibility to, | OPTN, GLC1E, FIP2, HYPL, NRP |
| 606657 (3) | |
| Glaucoma, primary open angle, adult-onset, | CYP1B1, GLC3A |
| 137760 (3) | |
| Glaucoma, primary open angle, juvenile- | CYP1B1, GLC3A |
| onset, 137750 (3) | |
| Glioblastoma, early-onset, 137800 (3) | MSH2, COCA1, FCC1, HNPCC1 |
| Glioblastoma multiforme, somatic, 137800 | DMBT1 |
| (3) | |
| Glioblastoma, somatic, 137800 (3) | ERBB2, NGL, NEU, HER2 |
| Glioblastoma, somatic, 137800 (3) | LGI1, EPT, ETL1 |
| Glioblastoma, susceptibility to, 137800 (3) | PPARG, PPARG1, PPARG2 |
| Glomerulocystic kidney disease, | TCF2, HNF2 |
| hypoplastic, 137920 (3) | |
| Glomerulosclerosis, focal segmental, 1, | ACTN4, FSGS1, FSGS |
| 603278 (3) | |
| Glomerulosclerosis, focal segmental, 2, | TRPC6, TRP6, FSGS2 |
| 603965 (3) | |
| Glomerulosclerosis, focal segmental, 3, | CD2AP, CMS |
| 607832 (3) | |
| Glomuvenous malformations, 138000 (3) | GLML, GVM, VMGLOM |
| Glucocorticoid deficiency 2, 607398 (3) | MRAP, FALP, C21orf61 |
| Glucocorticoid deficiency, due to ACTH | MC2R |
| unresponsiveness, 202200 (3) | |
| Glucose/galactose malabsorption, 606824 | SLC5A1, SGLT1 |
| (3) | |
| Glucose transport defect, blood-brain | SLC2A1, GLUT1 |
| barrier, 606777 (3) | |
| Glucosidase I deficiency, 606056 (3) | GCS1 |
| Glutamate formiminotransferase deficiency, | FTCD |
| 229100 (3) | |
| Glutaricaciduria, type I, 231670 (3) | GCDH |
| Glutaricaciduria, type IIA, 231680 (3) | ETFA, GA2, MADD |
| Glutaricaciduria, type IIB, 231680 (3) | ETFB, MADD |
| Glutaricaciduria, type IIC, 231680 (3) | ETFDH, MADD |
| Glutathione synthetase deficiency, 266130 | GSS, GSHS |
| (3) | |
| Glycerol kinase deficiency, 307030 (3) | GK |
| Glycine encephalopathy, 605899 (3) | AMT, NKH, GCE |
| Glycine encephalopathy, 605899 (3) | GCSH, NKH |
| Glycine encephalopathy, 605899 (3) | GLDC, HYGN1, GCSP, GCE, NKH |
| Glycine N-methyltransferase deficiency, | GNMT |
| 606664 (3) | |
| Glycogenosis, hepatic, autosomal (3) | PHKG2 |
| Glycogenosis, X-linked hepatic, type I (3) | PHKA2, PHK |
| Glycogenosis, X-linked hepatic, type II (3) | PHKA2, PHK |
| Glycogen storage disease I (3) | G6PC, G6PT |
| Glycogen storage disease Ib, 232220 (3) | G6PT1 |
| Glycogen storage disease Ic, 232240 (3) | G6PT1 |
| Glycogen storage disease II, 232300 (3) | GAA |
| Glycogen storage disease IIb, 300257 (3) | LAMP2, LAMPB |
| Glycogen storage disease IIIa (3) | AGL, GDE |
| Glycogen storage disease IIIb (3) | AGL, GDE |
| Glycogen storage disease IV, 232500 (3) | GBE1 |
| Glycogen storage disease, type 0, 240600 | GYS2 |
| (3) | |
| Glycogen storage disease VI (3) | PYGL |
| Glycogen storage disease VII (3) | PFKM |
| GM1-gangliosidosis (3) | GLB1 |
| GM2-gangliosidosis, AB variant (3) | GM2A |
| GM2-gangliosidosis, several forms, 272800 | HEXA, TSD |
| (3) | |
| Gnthodiaphyseal dysplasia, 166260 (3) | TMEM16E, GDD1 |
| Goiter, congenital (3) | TPO, TPX |
| Goiter, nonendemic, simple (3) | TG, AITD3 |
| Goldberg-Shprintzen megacolon syndrome, | KIAA1279 |
| 609460 (3) | |
| Gonadal dysgenesis, 46XY, partial, with | DHH |
| minifascicular neuropathy, 607080 (3) | |
| Gonadal dysgenesis, XY type (3) | SRY, TDF |
| GRACILE syndrome, 603358 (3) | BCS1L, FLNMS, GRACILE |
| Graft-versus-host disease, protection | IL10, CSIF |
| against (3) | |
| Graves disease, susceptibility to, 275000 (3) | CTLA4 |
| Graves disease, susceptibility to, 3, 275000 | GC, DBP |
| (3) | |
| Greenberg dysplasia, 215140 (3) | LBR, PHA |
| Greig cephalopolysyndactyly syndrome, | GLI3, PAPA, PAPB, ACLS |
| 175700 (3) | |
| Griscelli syndrome, type 1, 214450 (3) | MYO5A, MYH12, GS1 |
| Griscelli syndrome, type 2, 607624 (3) | RAB27A, RAM, GS2 |
| Griscelli syndrome, type 3, 609227 (3) | MLPH |
| Growth hormone deficient dwarfism (3) | GHRHR |
| Growth hormone insensitivity with | STAT5B |
| immunodeficiency, 245590 (3) | |
| Growth retardation with deafness and | IGF1 |
| mental retardation due to IGF1 deficiency, | |
| 608747 (3) | |
| Guttmacher syndrome, 176305 (3) | HOXA13, HOX1J |
| Gyrate atrophy of choroid and retina with | OAT |
| ornithinemia, B6 responsive or unresponsive | |
| (3) | |
| Hailey-Hailey disease, 169600 (3) | ATP2C1, BCPM, HHD |
| Haim-Munk syndrome, 245010 (3) | CTSC, CPPI, PALS, PLS, HMS |
| Hand-foot-uterus syndrome, 140000 (3) | HOXA13, HOX1J |
| Harderoporphyrinuria (3) | CPO |
| HARP syndrome, 607236 (3) | PANK2, NBIA1, PKAN, HARP |
| Hartnup disorder, 234500 (3) | SLC6A19, HND |
| Hay-Wells syndrome, 106260 (3) | TP73L, TP63, KET, EEC3, SHFM4, |
| | LMS, RHS |
| HDL deficiency, familial, 604091 (3) | ABCA1, ABC1, HDLDT1, TGD |
| HDL response to hormone replacement, | ESR1, ESR |
| augmented (3) | |
| Hearing loss, low-frequency sensorineural, | WFS1, WFRS, WFS, DFNA6 |
| 600965 (3) | |
| Heart block, nonprogressive, 113900 (3) | SCN5A, LQT3, IVF, HB1, SSS1 |
| Heart block, progressive, type I, 113900 (3) | SCN5A, LQT3, IVF, HB1, SSS1 |
| Heinz body anemia (3) | HBA2 |
| Heinz body anemias, alpha-(3) | HBA1 |
| Heinz body anemias, beta-(3) | HBB |
| HELLP syndrome, maternal, of pregnancy | HADHA, MTPA |
| (3) | |
| Hemangioblastoma, cerebellar, somatic (3) | VHL |
| Hemangioma, capillary infantile, somatic, | FLT4, VEGFR3, PCL |
| 602089 (3) | |
| Hemangioma, capillary infantile, somatic, | KDR |
| 602089 (3) | |
| Hematopoiesis, cyclic, 162800 (3) | ELA2 |
| Hematuria, familial benign (3) | COL4A4 |
| Heme oxygenase-1 deficiency (3) | HMOX1 |
| Hemiplegic migraine, familial, 141500 (3) | CACNA1A, CACNL1A4, SCA6 |
| Hemochromatosis (3) | HFE, HLA-H, HFE1 |
| Hemochromatosis, juvenile, 602390 (3) | HAMP, LEAP1, HEPC, HFE2 |
| Hemochromatosis, juvenile, digenic, 602390 | HAMP, LEAP1, HEPC, HFE2 |
| (3) | |
| Hemochromatosis, type 2A, 602390 (3) | HJV, HFE2A |
| Hemochromatosis, type 3, 604250 (3) | TFR2, HFE3 |
| Hemochromatosis, type 4, 606069 (3) | SLC40A1, SLC11A3, FPN1, IREG1, |
| | HFE4 |
| Hemoglobin H disease (3) | HBA2 |
| Hemolytic anemia due to adenylate kinase | AK1 |
| deficiency (3) | |
| Hemolytic anemia due to band 3 defect | SLC4A1, AE1, EPB3 |
| defect (3) | |
| Hemolytic anemia due to | BPGM |
| bisphosphoglycerate mutase deficiency (3) | |
| Hemolytic anemia due to G6PD deficiency | G6PD, G6PD1 |
| (3) | |
| Hemolytic anemia due to gamma- | GCLC, GLCLC |
| glutamylcysteine synthetase deficiency, | |
| 230450 (3) | |
| Hemolytic anemia due to glucosephosphate | GPI |
| isomerase deficiency (3) | |
| Hemolytic anemia due to glutathione | GSS, GSHS |
| synthetase deficiency, 231900 (3) | |
| Hemolytic anemia due to hexokinase | HK1 |
| deficiency (3) | |
| Hemolytic anemia due to PGK deficiency (3) | PGK1, PGKA |
| Hemolytic anemia due to triosephosphate | TPI1 |
| isomerase deficiency (3) | |
| Hemolytic-uremic syndrome, 235400 (3) | HF1, CFH, HUS |
| Hemophagocytic lymphohistiocytosis, | PRF1, HPLH2 |
| familial, 2, 603553 (3) | |
| Hemophagocytic lymphohistiocytosis, | UNC13D, MUNC13-4, HPLH3, HLH3, |
| familial, 3, 608898 (3) | FHL3 |
| Hemophilia A (3) | F8, F8C, HEMA |
| Hemophilia B (3) | F9, HEMB |
| Hemorrhagic diathesis due to | PI, AAT |
| \\|grave over ( )\|antithrombin\' Pittsburgh (3) | |
| Hemorrhagic diathesis due to factor V | F5 |
| deficiency (3) | |
| Hemosiderosis, systemic, due to | CP |
| aceruloplasminemia, 604290 (3) | |
| Hepatic adenoma, 142330 (3) | TCF1, HNF1A, MODY3 |
| Hepatic failure, early onset, and neurologic | SCOD1, SCO1 |
| disorder (3) | |
| Hepatic lipase deficiency (3) | LIPC |
| Hepatoblastoma (3) | CTNNB1 |
| Hepatocellular cancer, 114550 (3) | PDGFRL, PDGRL, PRLTS |
| Hepatocellular carcinoma, 114550 (3) | AXIN1, AXIN |
| Hepatocellular carcinoma, 114550 (3) | CTNNB1 |
| Hepatocellular carcinoma, 114550 (3) | TP53, P53, LFS1 |
| Hepatocellular carcinoma (3) | IGF2R, MPRI |
| Hepatocellular carcinoma, childhood type, | MET |
| 114550 (3) | |
| Hepatocellular carcinoma, somatic, 114550 | CASP8, MCH5 |
| (3) | |
| Hereditary hemorrhagic telangiectasia-1, | ENG, END, HHT1, ORW |
| 187300 (3) | |
| Hereditary hemorrhagic telangiectasia-2, | ACVRL1, ACVRLK1, ALK1, HHT2 |
| 600376 (3) | |
| Hereditary persistence of alpha-fetoprotein | AFP, HPAFP |
| (3) | |
| Hermansky-Pudlak syndrome, 203300 (3) | HPS1 |
| Hermansky-Pudlak syndrome, 203300 (3) | HPS3 |
| Hermansky-Pudlak syndrome, 203300 (3) | HPS4 |
| Hermansky-pudlak syndrome, 203300 (3) | HPS5, RU2, KIAA1017 |
| Hermansky-Pudlak syndrome, 203300 (3) | HPS6, RU |
| Hermansky-Pudlak syndrome, 608233 (3) | AP3B1, ADTB3A, HPS2 |
| Hermansky-Pudlak syndrome 7, 203300 (3) | DTNBP1, HPS7 |
| Heterotaxy, visceral, 605376 (3) | CFC1, CRYPTIC, HTX2 |
| Heterotaxy, X-linked visceral, 306955 (3) | ZIC3, HTX1, HTX |
| Heterotopia, periventricular, 300049 (3) | FLNA, FLN1, ABPX, NHBP, OPD1, |
| | OPD2, FMD, MNS |
| Heterotopia, periventricular, ED variant, | FLNA, FLN1, ABPX, NHBP, OPD1, |
| 300537 (3) | OPD2, FMD, MNS |
| Heterotopia, periventricular nodular, with | FLNA, FLN1, ABPX, NHBP, OPD1, |
| frontometaphyseal dysplasia, 300049 (3) | OPD2, FMD, MNS |
| Hex A pseudodeficiency, 272800 (3) | HEXA, TSD |
| High-molecular-weight kininogen deficiency | KNG |
| (3) | |
| Hirschsprung disease, 142623 (3) | EDN3 |
| Hirschsprung disease, 142623 (3) | GDNF |
| Hirschsprung disease, 142623 (3) | NRTN, NTN |
| Hirschsprung disease, 142623 (3) | RET, MEN2A |
| Hirschsprung disease-2, 600155 (3) | EDNRB, HSCR2, ABCDS |
| Hirschsprung disease, cardiac defects, and | ECE1 |
| autonomic dysfunction (3) | |
| Hirschsprung disease, short-segment, | PMX2B, NBPHOX, PHOX2B |
| 142623 (3) | |
| Histidinemia, 235800 (3) | HAL, HSTD |
| Histiocytoma (3) | TP53, P53, LFS1 |
| HIV-1 disease, delayed progression of (3) | CCL5, SCYA5, D17S136E, TCP228 |
| HIV-1 disease, rapid progression of (3) | CCL5, SCYA5, D17S136E, TCP228 |
| HIV-1, susceptibility to (3) | IL10, CSIF |
| HIV infection, susceptibility/resistance to (3) | CMKBR2, CCR2 |
| HIV infection, susceptibility/resistance to (3) | CMKBR5, CCCKR5 |
| HMG-CoA lyase deficiency (3) | HMGCL |
| HMG-CoA synthase-2 deficiency, 605911 | HMGCS2 |
| (3) | |
| Holocarboxylase synthetase deficiency, | HLCS, HCS |
| 253270 (3) | |
| Holoprosencephaly-2, 157170 (3) | SIX3, HPE2 |
| Holoprosencephaly-3, 142945 (3) | SHH, HPE3, HLP3, SMMCI |
| Holoprosencephaly-4, 142946 (3) | TGIF, HPE4 |
| Holoprosencephaly-5, 609637 (3) | ZIC2, HPE5 |
| Holoprosencephaly-7 (3) | PTCH, NBCCS, BCNS, HPE7 |
| Holt-Oram syndrome, 142900 (3) | TBX5 |
| Homocysteine, total plasma, elevated (3) | CTH |
| Homocystinuria, B6-responsive and | CBS |
| nonresponsive types (3) | |
| Homocystinuria due to MTHFR deficiency, | MTHFR |
| 236250 (3) | |
| Homocystinuria-megaloblastic anemia, cbl E | MTRR |
| type, 236270 (3) | |
| Homozygous 2p16 deletion syndrome, | SLC3A1, ATR1, D2H, NBAT |
| 606407 (3) | |
| Hoyeraal-Hreidarsson syndrome, 300240 | DKC1, DKC |
| (3) | |
| HPFH, deletion type (3) | HBB |
| HPFH, nondeletion type A (3) | HBG1 |
| HPFH, nondeletion type G (3) | HBG2 |
| HPRT-related gout, 300323 (3) | HPRT1, HPRT |
| H. pylori infection, susceptibility to, 600263 | IFNGR1 |
| (3) | |
| Huntington disease (3) | HD, IT15 |
| Huntington disease-like 1, 603218 (3) | PRNP, PRIP |
| Huntington disease-like 2, 606438 (3) | JPH3, JP3, HDL2 |
| Huntington disease-like-4, 607136 (3) | TBP, SCA17 |
| Hyalinosis, infantile systemic, 236490 (3) | ANTXR2, CMG2, JHF, ISH |
| Hydrocephalus due to aqueductal stenosis, | L1CAM, CAML1, HSAS1 |
| 307000 (3) | |
| Hydrocephalus with congenital idiopathic | L1CAM, CAML1, HSAS1 |
| intestinal pseudoobstruction, 307000 (3) | |
| Hydrocephalus with Hirschsprung disease | L1CAM, CAML1, HSAS1 |
| and cleft palate, 142623 (3) | |
| Hyperalphalipoproteinemia, 143470 (3) | CETP |
| Hyperammonemia with hypoornithinemia, | PYCS, GSAS |
| hypocitrullinemia, hypoargininemia, and | |
| hypoprolinemia (3) | |
| Hyperandrogenism, nonclassic type, due to | CYP21A2, CYP21, CA21H |
| 21-hydroxylase deficiency (3) | |
| Hyperapobetalipoproteinemia, susceptibility | PPARA, PPAR |
| to (3) | |
| Hyperbilirubinemia, familial transcient | UGT1A1, UGT1, GNT1 |
| neonatal, 237900 (3) | |
| Hypercalciuria, absorptive, susceptibility to, | SAC, HCA2 |
| 143870 (3) | |
| Hypercholanemia, familial, 607748 (3) | BAAT |
| Hypercholanemia, familial, 607748 (3) | EPHX1 |
| Hypercholanemia, familial, 607748 (3) | TJP2, ZO2 |
| Hypercholesterolemia, due to ligand- | APOB, FLDB |
| defective apo B, 144010 (3) | |
| Hypercholesterolemia, familial, 143890 (3) | LDLR, FHC, FH |
| Hypercholesterolemia, familial, 3, 603776 | PCSK9, NARC1, HCHOLA3, FH3 |
| (3) | |
| Hypercholesterolemia, familial, autosomal | ARH, FHCB2, FHCB1 |
| recessive, 603813 (3) | |
| Hypercholesterolemia, familial, due to LDLR | EPHX2 |
| defect, modifier of, 143890 (3) | |
| Hypercholesterolemia, familial, modification | APOA2 |
| of, 143890 (3) | |
| Hypercholesterolemia, susceptibility to, | GSBS |
| 143890 (3) | |
| Hypercholesterolemia, susceptibility to, | ITIH4, PK120, ITIHL1 |
| 143890 (3) | |
| Hyperekplexia and spastic paraparesis (3) | GLRA1, STHE |
| Hyperekplexia, autosomal recessive, | GLRB |
| 149400 (3) | |
| Hypereosinophilic syndrome, idiopathic, | PDGFRA |
| resistant to imatinib, 607685 (3) | |
| Hyperferritinemia-cataract syndrome, | FTL |
| 600886 (3) | |
| Hyper-IgD syndrome, 260920 (3) | MVK, MVLK |
| Hyperinsulinism, familial, 602485 (3) | GCK |
| Hyperinsulinism-hyperammonemia | GLUD1 |
| syndrome, 606762 (3) | |
| Hyperkalemic periodic paralysis, 170500 (3) | SCN4A, HYPP, NAC1A |
| Hyperkeratotic cutaneous capillary-venous | CCM1, CAM, KRIT1 |
| malformations associated with cerebral | |
| capillary malformations, 116860 (3) | |
| Hyperlipidemia, familial combined, | USF1, HYPLIP1 |
| susceptibility to, 602491 (3) | |
| Hyperlipoproteinemia, type Ib, 207750 (3) | APOC2 |
| Hyperlipoproteinemia, type III (3) | APOE, AD2 |
| Hyperlysinemia, 238700 (3) | AASS |
| Hypermethioninemia, persistent, autosomal | MAT1A, MATA1, SAMS1 |
| dominant, due to methionine | |
| adenosyltransferase I/III deficiency (3) | |
| Hypermethioninemia with deficiency of S- | AHCY, SAHH |
| adenosylhomocysteine hydrolase (3) | |
| Hyperornithinemia-hyperammonemia- | SLC25A15, ORNT1, HHH |
| homocitrullinemia syndrome, 238970 (3) | |
| Hyperostosis, endosteal, 144750 (3) | LRP5, BMND1, LRP7, LR3, OPPG, |
| | VBCH2 |
| Hyperoxaluria, primary, type I, 259900 (3) | AGXT, SPAT |
| Hyperoxaluria, primary, type II, 260000 (3) | GRHPR, GLXR |
| Hyperparathyroidism, AD, 145000 (3) | MEN1 |
| Hyperparathyroidism, familial primary, | HRPT2, C1orf28 |
| 145000 (3) | |
| Hyperparathyroidism-jaw tumor syndrome, | HRPT2, C1orf28 |
| 145001 (3) | |
| Hyperparathyroidism, neonatal, 239200 (3) | CASR, HHC1, PCAR1, FIH |
| Hyperphenylalaninemia due to pterin-4a- | PCBD, DCOH |
| carbinolamine dehydratase deficiency, | |
| 264070 (3) | |
| Hyperphenylalaninemia, mild (3) | PAH, PKU1 |
| Hyperproinsulinemia, familial (3) | INS |
| Hyperprolinemia, type I, 239500 (3) | PRODH, PRODH2, SCZD4 |
| Hyperprolinemia, type II, 239510 (3) | ALDH4A1, ALDH4, P5CDH |
| Hyperproreninemia (3) | REN |
| Hyperprothrombinemia (3) | F2 |
| Hypertension, diastolic, resistance to, | KCNMB1 |
| 608622 (3) | |
| Hypertension, early-onset, autosomal | NR3C2, MLR, MCR |
| dominant, with exacerbation in pregnancy, | |
| 605115 (3) | |
| Hypertension, essential, 145500 (3) | AGTR1, AGTR1A, AT2R1 |
| Hypertension, essential, 145500 (3) | PTGIS, CYP8A1, PGIS, CYP8 |
| Hypertension, essential, salt-sensitive, | ADD1 |
| 145500 (3) | |
| Hypertension, essential, susceptibility to, | AGT, SERPINA8 |
| 145500 (3) | |
| Hypertension, essential, susceptibility to, | ECE1 |
| 145500 (3) | |
| Hypertension, essential, susceptibility to, | GNB3 |
| 145500 (3) | |
| Hypertension, insulin resistance-related, | RETN, RSTN, FIZZ3 |
| susceptibility to, 125853 (3) | |
| Hypertension, mild low-renin (3) | HSD11B2, HSD11K |
| Hypertension, pregnancy-induced, 189800 | NOS3 |
| (3) | |
| Hypertension, salt-sensitive essential, | CYP3A5, P450PCN3 |
| susceptibility to, 145500 (3) | |
| Hypertension, susceptibility to, 145500 (3) | NOS3 |
| Hyperthroidism, congenital (3) | TSHR |
| Hyperthyroidism, congenital (3) | TPO, TPX |
| Hypertriglyceridemia, one form (3) | APOA1 |
| Hypertriglyceridemia, susceptibility to, | APOA5 |
| 145750 (3) | |
| Hypertriglyceridemia, susceptibility to, | LIPI, LPDL, PRED5 |
| 145750 (3) | |
| Hypertriglyceridemia, susceptibility to, | RP1, ORP1 |
| 145750 (3) | |
| Hypertrypsinemia, neonatal (3) | CFTR, ABCC7, CF, MRP7 |
| Hyperuricemic nephropathy, familial | UMOD, HNFJ, FJHN, MCKD2, |
| juvenile, 162000 (3) | ADMCKD2 |
| Hypoaldosteronism, congenital, due to CMO | CYP11B2 |
| I deficiency, 203400 (3) | |
| Hypoaldosteronism, congenital, due to CMO | CYP11B2 |
| II deficiency (3) | |
| Hypoalphalipoproteinemia (3) | APOA1 |
| Hypobetalipoproteinemia (3) | APOB, PLDB |
| Hypocalcemia, autosomal dominant, | CASR, HHC1, PCAR1, FIH |
| 146200 (3) | |
| Hypocalcemia, autosomal dominant, with | CASR, HHC1, PCAR1, FIH |
| Bartter syndrome (3) | |
| Hypocalciuric hypercalcemia, type I, 145980 | CASR, HHC1, PCAR1, FIH |
| (3) | |
| Hypoceruloplasminemia, hereditary, 604290 | CP |
| (3) | |
| Hypochondroplasia, 146000 (3) | FGFR3, ACH |
| Hypochromic microcytic anemia (3) | HBA2 |
| Hypodontia, 106600 (3) | PAX9 |
| Hypodontia, autosomal dominant, 106600 | MSX1, HOX7, HYD1, OFC5 |
| (3) | |
| Hypodontia with orofacial cleft, 106600 (3) | MSX1, HOX7, HYD1, OFC5 |
| Hypofibrinogenemia, gamma type (3) | FGG |
| Hypoglobulinemia and absent B cells (3) | BLNK, SLP65 |
| Hypoglycemia of infancy, leucine-sensitive, | ABCC8, SUR, PHHI, SUR1 |
| 240800 (3) | |
| Hypoglycemia of infancy, persistent | ABCC8, SUR, PHHI, SUR1 |
| hyperinsulinemic, 256450 (3) | |
| Hypogonadism, hypergonadotropic (3) | LHB |
| Hypogonadotropic hypogonadism, 146110 | GPR54 |
| (3) | |
| Hypogonadotropic hypogonadism, 146110 | NELF |
| (3) | |
| Hypogonadotropic hypogonadism (3) | GNRHR, LHRHR |
| Hypogonadotropic hypogonadism (3) | LHCGR |
| Hypohaptoglobinemia (3) | HP |
| Hypokalemic periodic paralysis, 170400 (3) | CACNA1S, CACNL1A3, CCHL1A3 |
| Hypokalemic periodic paralysis, 170400 (3) | KCNE3, HOKPP |
| Hypokalemic periodic paralysis, 170400 (3) | SCN4A, HYPP, NAC1A |
| Hypolactasia, adult type, 223100 (3) | LCT, LAC, LPH |
| Hypolactasia, adult type, 223100 (3) | MCM6 |
| Hypomagnesemia-2, renal, 154020 (3) | FXYD2, ATP1G1, HOMG2 |
| Hypomagnesemia, primary, 248250 (3) | CLDN16, PCLN1 |
| Hypomagnesemia with secondary | TRPM6, CHAK2 |
| hypocalcemia, 602014 (3) | |
| Hypoparathyroidism, autosomal dominant(3) | PTH |
| Hypoparathyroidism, autosomal recessive | PTH |
| (3) | |
| Hypoparathyroidism, familial isolated, | GCMB |
| 146200 (3) | |
| Hypoparathyroidism-retardation- | TBCE, KCS, KCS1, HRD |
| dysmorphism syndrome, 241410 (3) | |
| Hypoparathyroidism, sensorineural | GATA3, HDR |
| deafness, and renal dysplasia, 146255 (3) | |
| Hypophosphatasia, childhood, 241510 (3) | ALPL, HOPS, TNSALP |
| Hypophosphatasia, infantile, 241500 (3) | ALPL, HOPS, TNSALP |
| Hypophosphatemia, type III (3) | CLCN5, CLCK2, NPHL2, DENTS |
| Hypophosphatemia, X-linked, 307800 (3) | PHEX, HYP, HPDR1 |
| Hypophosphatemic rickets, autosomal | FGF23, ADHR, HPDR2, PHPTC |
| dominant, 193100 (3) | |
| Hypoplastic enamel pitting, localized, | ENAM |
| 608563 (3) | |
| Hypoplastic left heart syndrome, 241550 (3) | GJA1, CX43, ODDD, SDTY3, ODOD |
| Hypoprothrombinemia (3) | F2 |
| Hypothyroidism, autoimmune, 140300 (3) | CTLA4 |
| Hypothyroidism, congenital, 274400 (3) | SLC5A5, NIS |
| Hypothyroidism, congenital, due to DUOX2 | DUOX2, THOX2 |
| deficiency, 607200 (3) | |
| Hypothyroidism, congenital, due to thyroid | PAX8 |
| dysgenesis or hypoplasia, 218700 (3) | |
| Hypothyroidism, congenital, due to TSH | TSHR |
| resistance, 275200 (3) | |
| Hypothyroidism, hereditary congenital (3) | TG, AITD3 |
| Hypothyroidism, nongoitrous (3) | TSHB |
| Hypothyroidism, subclinical (3) | TSHR |
| Hypotrichosis, congential, with juvenile | CDH3, CDHP, PCAD, HJMD |
| macular dystrophy, 601553 (3) | |
| Hypotrichosis, localized, autosomal | DSG4, LAH |
| recessive, 607903 (3) | |
| Hypotrichosis-lymphedema-telangiectasia | SOX18, HLTS |
| syndrome, 607823 (3) | |
| Hypotrichosis simplex of scalp, 146520 (3) | CDSN, HTSS |
| Hypouricemia, renal, 220150 (3) | SLC22A12, OAT4L, URAT1 |
| Hystrix-like ichthyosis with deafness, | GJB2, CX26, DFNB1, PPK, DFNA3, |
| 602540 (3) | KID, HID |
| Ichthyosiform erythroderma, congenital, | TGM1, ICR2, LI1 |
| 242100 (3) | |
| Ichthyosiform erythroderma, congenital, | ALOX12B |
| nonbullous, 1, 242100 (3) | |
| Ichthyosiform erythroderma, congenital, | ALOXE3 |
| nonbullous, 1, 242100 (3) | |
| Ichthyosis bullosa of Siemens, 146800 (3) | KRT2A, KRT2E |
| Ichthyosis, congenital, autosomal recessive | ICHYN |
| (3) | |
| Ichthyosis, cyclic, with epidermolytic | KRT10 |
| hyperkeratosis, 607602 (3) | |
| Ichthyosis, harlequin, 242500 (3) | ABCA12, ICR2B, LI2 |
| Ichthyosis histrix, Curth-Macklin type, | KRT1 |
| 146590 (3) | |
| Ichthyosis, lamellar 2, 601277 (3) | ABCA12, ICR2B, LI2 |
| Ichthyosis, lamellar, autosomal recessive, | TGM1, ICR2, LI1 |
| 242300 (3) | |
| Ichthyosis, X-linked (3) | STS, ARSC1, ARSC, SSDD |
| ICOS deficiency, 607594 (3) | ICOS, AILIM |
| IgE levels QTL, 147050 (3) | PHF11, NYREN34 |
| IgG2 deficiency, selective (3) | IGHG2 |
| IgG receptor 1, phagocytic, familial | FCGR1A, IGFR1, CD64 |
| deficiency of (3) | |
| Immunodeficiency-centromeric instability- | DNMT3B, ICF |
| facial anomalies syndrome, 242860 (3) | |
| Immunodeficiency due to defect in CD3- | CD3E |
| epsilon (3) | |
| Immunodeficiency due to defect in CD3- | CD3G |
| gamma (3) | |
| Immunodeficiency with hyper-IgM, type 2, | AICDA, AID, HIGM2 |
| 605258 (3) | |
| Immunodeficiency with hyper-IgM, type 3, | TNFRSF5, CD40 |
| 606843 (3) | |
| Immunodeficiency with hyper IgM, type 4, | UNG, DGU, HIGM4 |
| 608106 (3) | |
| Immunodeficiency, X-linked, with hyper-IgM, | TNFSF5, CD40LG, HIGM1, IGM |
| 308230 (3) | |
| Immunodysregulation, polyendocrinopathy, | FOXP3, IPEX, AHD, XPID, PIDX |
| and enteropathy, X-linked, 304790 (3) | |
| Immunoglobulin A deficiency, 609529 (3) | TNFRSF14B, TACI |
| Inclusion body myopathy-3, 605637 (3) | MYH2 |
| Inclusion body myopathy, autosomal | GNE, GLCNE, IBM2, DMRV, NM |
| recessive, 600737 (3) | |
| Inclusion body myopathy with early-onset | VCP, IBMPFD |
| Paget disease and frontotemporal dementia, | |
| 167320 (3) | |
| Incontinentia pigmenti, type II, 308300 (3) | IKBKG, NEMO, FIP3, IP2 |
| Infantile spasm syndrome, 308350 (3) | ARX, ISSX, PRTS, MRXS1, MRX36, |
| | MRX54 |
| Infundibular hypoplasia and hypopituitarism | SOX3, MRGH |
| (3) | |
| Inosine triphosphatase deficiency (3) | ITPA |
| Insensitivity to pain, congenital, with | NTRK1, TRKA, MTC |
| anhidrosis, 256800 (3) | |
| Insomnia (3) ( ) | GABRB3 |
| Insomnia, fatal familial, 600072 (3) | PRNP, PRIP |
| Insulin resistance, severe, digenic, 604367 | PPARG, PPARG1, PPARG2 |
| (3) | |
| Insulin resistance, severe, digenic, 604367 | PPP1R3A, PPP1R3 |
| (3) | |
| Insulin resistance, susceptibility to (3) | PTPN1, PTP1B |
| Interleukin-2 receptor, alpha chain, | IL2RA, IL2R |
| deficiency of (3) | |
| Intervertebral disc disease, susceptibility to, | COL9A2, EDM2 |
| 603932 (3) | |
| Intervertebral disc disease, susceptibility to, | COL9A3, EDM3, IDD |
| 603932 (3) | |
| Intrauterine and postnatal growth retardation | IGF1R |
| (3) | |
| Intrauterine and postnatal growth retardation | IGF2 |
| (3) | |
| Intrinsic factor deficiency, 261000 (3) | GIF, IF |
| IRAK4 deficiency, 607676 (3) | IRAK4, REN64 |
| Iridogoniodysgenesis, 601631 (3) | FOXC1, FKHL7, FREAC3 |
| Iridogoniodysgenesis syndrome-2, 137600 | PITX2, IDG2, RIEG1, RGS, IGDS2 |
| (3) | |
| Iris hypoplasia and glaucoma (3) | FOXC1, FKHL7, FREAC3 |
| Iron deficiency anemia, susceptibility to (3) | TF |
| Iron overload, autosomal dominant (3) | FTH1, FTHL6 |
| Isolated growth hormone deficiency, Illig | GH1, GHN |
| type with absent GH and Kowarski type with | |
| bioinactive GH (3) | |
| Isovaleric acidemia, 243500 (3) | IVD |
| Jackson-Weiss syndrome, 123150 (3) | FGFR1, FLT2, KAL2 |
| Jackson-Weiss syndrome, 123150 (3) | FGFR2, BEK, CFD1, JWS |
| Jensen syndrome, 311150 (3) | TIMM8A, DFN1, DDP, MTS, DDP1 |
| Jervell and Lange-Nielsen syndrome, | KCNE1, JLNS, LQT5 |
| 220400 (3) | |
| Jervell and Lange-Nielsen syndrome, | KCNQ1, KCNA9, LQT1, KVLQT1, |
| 220400 (3) | ATFB1 |
| Joubert syndrome, 213300 (3) | NPHP1, NPH1, SLSN1 |
| Joubert syndrome-3, 608629 (3) | AHI1 |
| Juberg-Marsidi syndrome, 309590 (3) | ATRX, XH2, XNP, MRXS3, SHS |
| Juvenile polyposis/hereditary hemorrhagic | MADH4, DPC4, SMAD4, JIP |
| telangiectasia syndrome, 175050 (3) | |
| Kallikrein, decreased urinary activity of (3) | KLK1, KLKR |
| Kallmann syndrome 2, 147950 (3) | FGFR1, FLT2, KAL2 |
| Kallmann syndrome (3) | KALI, KMS, ADMLX |
| Kanzaki disease, 609242 (3) | NAGA |
| Kaposi sarcoma, susceptibility to, 148000 | IL6, IFNB2, BSF2 |
| (3) | |
| Kappa light chain deficiency (3) | IGKC |
| Kartagener syndrome, 244400 (3) | DNAH11, DNAHC11 |
| Kartagener syndrome, 244400 (3) | DNAH5, HL1, PCD, CILD3 |
| Kartagener syndrome, 244400 (3) | DNAI1, CILD1, ICS, PCD |
| Kenny-Caffey syndrome-1, 244460 (3) | TBCE, KCS, KCS1, HRD |
| Keratitis, 148190 (3) | PAX6, AN2, MGDA |
| Keratitis-ichthyosis-deafness syndrome, | GJB2, CX26, DFNB1, PPK, DFNA3, |
| 148210 (3) | KID, HID |
| Keratoconus, 148300 (3) | VSX1, RINX, PPCD, PPD, KTCN |
| Keratoderma, palmoplantar, with deafness, | GJB2, CX26, DFNB1, PPK, DFNA3, |
| 148350 (3) | KID, HID |
| Keratosis follicularis spinulosa decalvans, | SAT, SSAT, KFSD |
| 308800 (3) | |
| Keratosis palmoplantaria striata, 148700 (3) | KRT1 |
| Keratosis palmoplantaris striata I, 148700 | DSG1 |
| (3) | |
| Keratosis palmoplantaris striata II (3) | DSP, KPPS2, PPKS2 |
| Keratosis palmoplantaris striata III, 607654 | KRT1 |
| (3) | |
| Ketoacidosis due to SCOT deficiency (3) | SCOT, OXCT |
| Keutel syndrome, 245150 (3) | MGP, NTI |
| Kindler syndrome, 173650 (3) | KIND1, URP1, C20orf42 |
| Kininogen deficiency (3) | KNG |
| Klippel-Trenaunay syndrome, 149000 (3) | VG5Q, HUS84971, FLJ10283 |
| Kniest dysplasia, 156550 (3) | COL2A1 |
| Knobloch syndrome, 267750 (3) | COL18A1, KNO |
| Krabbe disease, 245200 (3) | GALC |
| L-2-hydroxyglutaric aciduria, 236792 (3) | L2HGDH, C14orf160 |
| Lactate dehydrogenase-B deficiency (3) | LDHB |
| Lacticacidemia due to PDX1 deficiency, | PDX1 |
| 245349 (3) | |
| Langer mesomelic dysplasia, 249700 (3) | SHOX, GCFX, SS, PHOG |
| Langer mesomelic dysplasia, 249700 (3) | SHOXY |
| Laron dwarfism, 262500 (3) | GHR |
| Larson syndrome, 150250 (3) | FLNB, SCT, AOI |
| Laryngoonychocutaneous syndrome, | LAMA3, LOCS |
| 245660 (3) | |
| Lathosterolosis, 607330 (3) | SC5DL, ERG3 |
| LCHAD deficiency (3) | HADHA, MTPA |
| Lead poisoning, susceptibility to (3) | ALAD |
| Leanness, inherited (3) | AGRP, ART, AGRT |
| Leber congenital amaurosis, 204000 (3) | CRB1, RP12 |
| Leber congenital amaurosis, 204000 (3) | CRX, CORD2, CRD |
| Leber congenital amaurosis, 204000 (3) | RPGRIP1, LCA6, CORD9 |
| Leber congenital amaurosis-2, 204100 (3) | RPE65, RP20 |
| Leber congenital amaurosis, 604393 (3) | AIPL1, LCA4 |
| Leber congenital amaurosis, type I, 204000 | GUCY2D, GUC2D, LCA1, CORD6 |
| (3) | |
| Leber congenital amaurosis, type III, | RDH12, LCA3 |
| 604232 (3) | |
| Left-right axis malformations (3) | ACVR2B |
| Left-right axis malformations (3) | EBAF, TGFB4, LEFTY2, LEFTA, |
| | LEFTYA |
| Left ventricular noncompaction, familial | DTNA, D18S892E, DRP3, LVNC1 |
| isolated, 1, 604169 (3) | |
| Left ventricular noncompaction with | DTNA, D18S892E, DRP3, LVNC1 |
| congenital heart defects, 606617 (3) | |
| Legionaire disease, susceptibility to, 608556 | TLR5, TIL3 |
| (3) | |
| Leigh syndrome, 256000 (3) | BCS1L, FLNMS, GRACILE |
| Leigh syndrome, 256000 (3) | DLD, LAD, PHE3 |
| Leigh syndrome, 256000 (3) | NDUFS3 |
| Leigh syndrome, 256000 (3) | NDUFS4, AQDQ |
| Leigh syndrome, 256000 (3) | NDUFS7, PSST |
| Leigh syndrome, 256000 (3) | NDUFS8 |
| Leigh syndrome, 256000 (3) | NDUFV1, UQOR1 |
| Leigh syndrome, 256000 (3) | SDHA, SDH2, SDHF |
| Leigh syndrome, due to COX deficiency, | SURF1 |
| 256000 (3) | |
| Leigh syndrome due to cytochrome c | COX15 |
| oxidase deficiency, 256000 (3) | |
| Leigh syndrome, French-Canadian type, | LRPPRC, LRP130, LSFC |
| 220111 (3) | |
| Leigh syndrome, X-linked, 308930 (3) | PDHA1, PHE1A |
| Leiomyomatosis and renal cell cancer, | FH |
| 605839 (3) | |
| Leiomyomatosis, diffuse, with Alport | COL4A6 |
| syndrome, 308940 (3) | |
| Leopard syndrome, 151100 (3) | PTPN11, PTP2C, SHP2, NS1 |
| Leprechaunism, 246200 (3) | INSR |
| Leprosy, susceptibility to, 607572 (3) | PRKN, PARK2, PDJ |
| Leri-Weill dyschondrosteosis, 127300 (3) | SHOX, GCFX, SS, PHOG |
| Leri-Weill dyschondrosteosis, 127300 (3) | SHOXY |
| Lesch-Nyhan syndrome, 300322, (3) | HPRT1, HPRT |
| Leukemia-1, T-cell acute lymphocytic (3) | TAL1, TCL5, SCL |
| Leukemia-2, T-cell acute lymphoblastic (3) | TAL2 |
| Leukemia, acute lymphoblastic (3) | FLT3 |
| Leukemia, acute lymphoblastic (3) | NBS1, NBS |
| Leukemia, acute lymphoblastic (3) | ZNFN1A1, IK1, LYF1 |
| Leukemia, acute lymphoblastic, | HOXD4, HOX4B |
| susceptibility to (3) | |
| Leukemia, acute lymphocytic (3) | BCR, CML, PHL, ALL |
| Leukemia, acute myeloblastic (3) | ARNT |
| Leukemia, acute myelogenous (3) | KRAS2, RASK2 |
| Leukemia, acute myelogenous, 601626 (3) | GMPS |
| Leukemia, acute myeloid, 601626 (3) | AF10 |
| Leukemia, acute myeloid, 601626 (3) | ARHGEF12, LARG, KIAA0382 |
| Leukemia, acute myeloid, 601626 (3) | CALM, CLTH |
| Leukemia, acute myeloid, 601626 (3) | CEBPA, CEBP |
| Leukemia, acute myeloid, 601626 (3) | CHIC2, BTL |
| Leukemia, acute myeloid, 601626 (3) | FLT3 |
| Leukemia, acute myeloid, 601626 (3) | KIT, PBT |
| Leukemia, acute myeloid, 601626 (3) | LPP |
| Leukemia, acute myeloid, 601626 (3) | NPM1 |
| Leukemia, acute myeloid, 601626 (3) | NUP214, D9S46E, CAN, CAIN |
| Leukemia, acute myeloid, 601626 (3) | RUNX1, CBFA2, AML1 |
| Leukemia, acute myeloid, 601626 (3) | WHSC1L1, NSD3 |
| Leukemia, acute myeloid, reduced survival | FLT3 |
| in (3) | |
| Leukemia, acute myelomonocytic (3) | AF1Q |
| Leukemia, acute promyelocytic, NPM/RARA | NPM1 |
| type (3) | |
| Leukemia, acute promyelocytic, | NUMA1 |
| NUMA/RARA type (3) | |
| Leukemia, acute promyelocytic, | ZNF145, PLZF |
| PL2F/RARA type (3) | |
| Leukemia, acute promyelocytic, PML/RARA | PML, MYL |
| type (3) | |
| Leukemia, acute promyeloyctic, | STAT5B |
| STAT5B/RARA type (3) | |
| Leukemia, acute T-cell lymphoblastic (3) | AF10 |
| Leukemia, acute T-cell lymphoblastic (3) | CALM, CLTH |
| Leukemia, chronic lymphatic, susceptibility | ARL11, ARLTS1 |
| to, 151400 (3) | |
| Leukemia, chronic lymphatic, susceptibility | P2RX7, P2X7 |
| to, 151400 (3) | |
| Leukemia, chronic myeloid, 608232 (3) | BCR, CML, PHL, ALL |
| Leukemia, juvenile myelomonocytic, 607785 | GRAF |
| (3) | |
| Leukemia, juvenile myelomonocytic, 607785 | NF1, VRNF, WSS, NFNS |
| (3) | |
| Leukemia, juvenile myelomonocytic, 607785 | PTPN11, PTP2C, SHP2, NS1 |
| (3) | |
| Leukemia/lymphoma, B-cell, 2 (3) | BCL2 |
| Leukemia/lymphoma, chronic B-cell, 151400 | CCND1, PRAD1, BCL1 |
| (3) | |
| Leukemia/lymphoma, T-cell (3) | TCRA |
| Leukemia, megakaryoblastic, of Down | GATA1, GF1, ERYF1, NFE1 |
| syndrome, 190685 (3) | |
| Leukemia, megakaryoblastic, with or without | GATA1, GF1, ERYF1, NFE1 |
| Down syndrome, 190685 (3) | |
| Leukemia, Philadelphia chromosome- | ABL1 |
| positive, resistant to imatinib (3) | |
| Leukemia, post-chemotherapy, susceptibility | NQO1, DIA4, NMOR1 |
| to (3) | |
| Leukemia, T-cell acute lymphoblastic (3) | NUP214, D9S46E, CAN, CAIN |
| Leukocyte adhesion deficiency, 116920 (3) | ITGB2, CD18, LCAMB, LAD |
| Leukoencephalopathy with vanishing white | EIF2B1, EIF2BA |
| matter, 603896 (3) | |
| Leukoencephalopathy with vanishing white | EIF2B2 |
| matter, 603896 (3) | |
| Leukoencephalopathy with vanishing white | EIF2B3 |
| matter, 603896 (3) | |
| Leukoencephalopathy with vanishing white | EIF2B5, LVWM, CACH, CLE |
| matter, 603896 (3) | |
| Leukoencephaly with vanishing white | EIF2B4 |
| matter, 603896 (3) | |
| Leydig cell adenoma, with precocious | LHCGR |
| puberty (3) | |
| Lhermitte-Duclos syndrome (3) | PTEN, MMAC1 |
| Liddle syndrome, 177200 (3) | SCNN1B |
| Liddle syndrome, 177200 (3) | SCNN1G, PHA1 |
| Li Fraumeni syndrome, 151623 (3) | CDKN2A, MTS1, P16, MLM, CMM2 |
| Li-Fraumeni syndrome, 151623 (3) | TP53, P53, LFS1 |
| Li-Fraumeni syndrome, 609265 (3) | CHEK2, RAD53, CHK2, CDS1, LFS2 |
| LIG4 syndrome, 606593 (3) | LIG4 |
| Limb-mammary syndrome, 603543 (3) | TP73L, TP63, KET, EEC3, SHFM4, |
| | LMS, RHS |
| Lipodystrophy, congenital generalized, type | AGPAT2, LPAAB, BSCL, BSCL1 |
| 1, 608594 (3) | |
| Lipodystrophy, congenital generalized, type | BSCL2, SPG17 |
| 2, 269700 (3) | |
| Lipodystrophy, familial partial, 151660 (3) | LMNA, LMN1, EMD2, FPLD, CMD1A, |
| | HGPS, LGMD1B |
| Lipodystrophy, familial partial, 151660 (3) | PPARG, PPARG1, PPARG2 |
| Lipodystrophy, familial partial, with | PPARGC1A, PPARGC1 |
| decreased subcutaneous fat of face and | |
| neck (3) | |
| Lipoid adrenal hyperplasia, 201710 (3) | STAR |
| Lipoid congenital adrenal hyperplasia, | CYP11A, P450SCC |
| 201710 (3) | |
| Lipoid proteinosis, 247100 (3) | ECM1 |
| Lipoma (3) | HMGA2, HMGIC, BABL, LIPO |
| Lipoma (3) | LPP |
| Lipoma, sporadic (3) | MEN1 |
| Lipomatosis, mutiple, 151900 (3) | HMGA2, HMGIC, BABL, LIPO |
| Lipoprotein lipase deficiency (3) | LPL, LIPD |
| Lissencephaly-1, 607432 (3) | PAFAH1B1, LIS1 |
| Lissencephaly syndrome, Norman-Roberts | RELN, RL |
| type, 257320 (3) | |
| Lissencephaly, X-linked, 300067 (3) | DCX, DBCN, LISX |
| Lissencephaly, X-linked with ambiguous | ARX, ISSX, PRTS, MRXS1, MRX36, |
| genitalia, 300215 (3) | MRX54 |
| Listeria monocytogenes, susceptibility to (3) | CDH1, UVO |
| Loeys-Dietz syndrome, 609192 (3) | TGFBR1 |
| Loeys-Dietz syndrome, 609192 (3) | TGFBR2, HNPCC6 |
| Longevity, exceptional, 152430 (3) | CETP |
| Longevity, reduced, 152430 (3) | AKAP10 |
| Long QT syndrome-1, 192500 (3) | KCNQ1, KCNA9, LQT1, KVLQT1, |
| | ATFB1 |
| Long QT syndrome-2 (3) | KCNH2, LQT2, HERG |
| Long QT syndrome-3, 603830 (3) | SCN5A, LQT3, IVF, HB1, SSS1 |
| Long QT syndrome 4, 600919 (3) | ANK2, LQT4 |
| Long QT syndrome-5 (3) | KCNE1, JLNS, LQT5 |
| Long QT syndrome-6 (3) | KCNE2, MIRP1, LQT6 |
| Long QT syndrome-7, 170390 (3) | KCNJ2, HHIRK1, KIR2.1, IRK1, LQT7 |
| Lower motor neuron disease, progressive, | DCTN1 |
| without sensory symptoms, 607641 (3) | |
| Lowe syndrome, 309000 (3) | OCRL, LOCR, OCRL1, NPHL2 |
| Low renin hypertension, susceptibility to (3) | CYP11B2 |
| LPA deficiency, congenital (3) | LPA |
| Lumbar disc disease, susceptibility to, | CILP |
| 603932 (3) | |
| Lung cancer, 211980 (3) | KRAS2, RASK2 |
| Lung cancer, 211980 (3) | PPP2R1B |
| Lung cancer, 211980 (3) | SLC22A1L, BWSCR1A, IMPT1 |
| Lung cancer, somatic, 211980 (3) | MAP3K8, COT, EST, TPL2 |
| Lupus nephritis, susceptibility to (3) | FCGR2A, IGFR2, CD32 |
| Lymphangioleiomyomatosis, 606690 (3) | TSC1, LAM |
| Lymphangioleiomyomatosis, somatic, | TSC2, LAM |
| 606690 (3) | |
| Lymphedema and ptosis, 153000 (3) | FOXC2, FKHL14, MFH1 |
| Lymphedema-distichiasis syndrome, | FOXC2, FKHL14, MFH1 |
| 153400 (3) | |
| Lymphedema-distichiasis syndrome with | FOXC2, FKHL14, MFH1 |
| renal disease and diabetes mellitus (3) | |
| Lymphedema, hereditary I, 153100 (3) | FLT4, VEGFR3, PCL |
| Lymphedema, hereditary II, 153200 (3) | FOXC2, FKHL14, MFH1 |
| Lymphocytic leukemia, acute T-cell (3) | RAP1GDS1 |
| Lymphoma, B-cell non-Hodgkin, somatic (3) | ATM, ATA, AT1 |
| Lymphoma, diffuse large cell (3) | BCL8 |
| Lymphoma, follicular (3) | BCL10 |
| Lymphoma, MALT (3) | BCL10 |
| Lymphoma, mantle cell (3) | ATM, ATA, AT1 |
| Lymphoma, non-Hodgkin (3) | RAD54B |
| Lymphoma, non-Hodgkin (3) | RAD54L, HR54, HRAD54 |
| Lymphoma, progression of (3) | FCGR2B, CD32 |
| Lymphoma, somatic (3) | MAD1L1, TXBP181 |
| Lymphoma, T-cell (3) | MSH2, COCA1, FCC1, HNPCC1 |
| Lymphoproliferative syndrome, X-linked, | SH2D1A, LYP, IMD5, XLP, XLPD |
| 308240 (3) | |
| Lynch cancer family syndrome II, 114400 | MSH2, COCA1, FCC1, HNPCC1 |
| (3) | |
| Lysinuric protein intolerance, 222700 (3) | SLC7A7, LPI |
| Machado-Joseph disease, 109150 (3) | ATXN3, MJD, SCA3 |
| Macrocytic anemia, refractory, of 5q- | IRF1, MAR |
| syndrome, 153550 (3) | |
| Macrothrombocytopenia, 300367 (3) | GATA1, GF1, ERYF1, NFE1 |
| Macular corneal dystrophy, 217800 (3) | CHST6, MCDC1 |
| Macular degeneration, age-related, 1, | HF1, CFH, HUS |
| 603075 (3) | |
| Macular degeneration, age-related, 1, | HMCN1, FBLN6, FIBL6 |
| 603075 (3) | |
| Macular degeneration, age-related, 3, | FBLN5, ARMD3 |
| 608895 (3) | |
| Macular degeneration, juvenile, 248200 (3) | CNGB3, ACHM3 |
| Macular degeneration, X-linked atrophic (3) | RPGR, RP3, CRD, RP15, COD1 |
| Macular dystrophy (3) | RDS, RP7, PRPH2, PRPH, AVMD, |
| | AOFMD |
| Macular dystrophy, age-related, 2, 153800 | ABCA4, ABCR, STGD1, FFM, RP19 |
| (3) | |
| Macular dystrophy, autosomal dominant, | ELOVL4, ADMD, STGD2, STGD3 |
| chromosome 6-linked, 600110 (3) | |
| Macular dystrophy, vitelliform, 608161 (3) | RDS, RP7, PRPH2, PRPH, AVMD, |
| | AOFMD |
| Macular dystrophy, vitelliform type, 153700 | VMD2 |
| (3) | |
| Maculopathy, bull's-eye, 153870 (3) | VMD2 |
| Major depressive disorder and accelerated | FKBP5, FKBP51 |
| response to antidepressant drug treatment, | |
| 608616 (3) | |
| Malaria, cerebral, reduced risk of, 248310 | CD36 |
| (3) | |
| Malaria, cerebral, susceptibility to, 248310 | CD36 |
| (3) | |
| Malaria, cerebral, susceptibility to (3) | ICAM1 |
| Malaria, cerebral, susceptibility to (3) | TNF, TNFA |
| Malaria, resistance to, 248310 (3) | GYPC, GE, GPC |
| Malaria, resistance to, 248310 (3) | NOS2A, NOS2 |
| Malignant hyperthermia susceptibility 1, | RYR1, MHS, CCO |
| 145600 (3) | |
| Malignant hyperthermia susceptibility 5, | CACNA1S, CACNL1A3, CCHL1A3 |
| 601887 (3) | |
| Malonyl-CoA decarboxylase deficiency, | MLYCD, MCD |
| 248360 (3) | |
| MALT lymphoma (3) | MALT1, MLT |
| Mandibuloacral dysplasia with type B | ZMPSTE24, FACE1, STE24, MADB |
| lipodystrophy, 608612 (3) | |
| Mannosidosis, alpha-, types I and II, 248500 | MAN2B1, MANB |
| (3) | |
| Mannosidosis, beta, 248510 (3) | MANBA, MANB1 |
| Maple syrup urine disease, type Ia, 248600 | BCKDHA, MSUD1 |
| (3) | |
| Maple syrup urine disease, type Ib (3) | BCKDHB, E1B |
| Maple syrup urine disease, type II (3) | DBT, BCATE2 |
| Maple syrup urine disease, type III, 248600 | DLD, LAD, PHE3 |
| (3) | |
| Marfan syndrome, 154700 (3) | FBN1, MFS1, WMS |
| Marfan syndrome, atypical (3) | COL1A2 |
| Maroteaux-Lamy syndrome, several forms | ARSB, MPS6 |
| (3) | |
| Marshall syndrome, 154780 (3) | COL11A1, STL2 |
| MASA syndrome, 303350 (3) | L1CAM, CAML1, HSAS1 |
| MASP2 deficiency (3) | MASP2 |
| MASS syndrome, 604308 (3) | FBN1, MFS1, WMS |
| Mast cell leukemia (3) | KIT, PBT |
| Mastocytosis with associated hematologic | KIT, PBT |
| disorder (3) | |
| Mast syndrome, 248900 (3) | ACP33, MAST, SPG21 |
| May-Hegglin anomaly, 155100 (3) | MYH9, MHA, FTNS, DFNA17 |
| McArdle disease, 232600 (3) | PYGM |
| McCune-Albright syndrome, 174800 (3) | GNAS, GNAS1, GPSA, POH, PHP1B, |
| | PHP1A, AHO |
| McKusick-Kaufman syndrome, 236700 (3) | MKKS, HMCS, KMS, MKS, BBS6 |
| McLeod syndrome (3) | XK |
| McLeod syndrome with neuroacanthosis (3) | XK |
| Medullary cystic kidney disease 2, 603860 | UMOD, HNFJ, FJHN, MCKD2, |
| (3) | ADMCKD2 |
| Medullary thyroid carcinoma, 155240 (3) | RET, MEN2A |
| Medullary thyroid carcinoma, familial, | NTRK1, TRKA, MTC |
| 155240 (3) | |
| Medulloblastoma, 155255 (3) | PTCH2 |
| Medulloblastoma, desmoplastic, 155255 (3) | SUFU, SUFUXL, SUFUH |
| Meesmann corneal dystrophy, 122100 (3) | KRT12 |
| Meesmann corneal dystrophy, 122100 (3) | KRT3 |
| Megakaryoblastic leukemia, acute (3) | MKL1, AMKL, MAL |
| Megalencephalic leukoencephalopathy with | MLC1, LVM, VL |
| subcortical cysts, 604004 (3) | |
| Megaloblastic anemia-1, Finnish type, | CUBN, IFCR, MGA1 |
| 261100 (3) | |
| Megaloblastic anemia-1, Norwegian type, | AMN |
| 261100 (3) | |
| Melanoma (3) | CDK4, CMM3 |
| Melanoma and neural system tumor | CDKN2A, MTS1, P16, MLM, CMM2 |
| syndrome, 155755 (3) | |
| Melanoma, cutaneous malignant, 2, 155601 | CDKN2A, MTS1, P16, MLM, CMM2 |
| (3) | |
| Melanoma, cutaneous malignant, | XRCC3 |
| susceptibility to (3) | |
| Melanoma, malignant sporadic (3) | STK11, PJS, LKB1 |
| Melanoma, melignant, somatic (3) | BRAF |
| Meleda disease, 248300 (3) | SLURP1, MDM |
| Melnick-Needles syndrome, 309350 (3) | FLNA, FLN1, ABPX, NHBP, OPD1, |
| | OPD2, FMD, MNS |
| Melorheostosis with osteopoikilosis, 155950 | LEMD3, MAN1 |
| (3) | |
| Memory impairment, susceptibility to (3) | BDNF |
| Meniere disease 156000 (3) ( ) | COCH, DFNA9 |
| Meningioma, 607174 (3) | MN1, MGCR |
| Meningioma, 607174 (3) | PTEN, MMAC1 |
| Meningioma, NF2-related, somatic, 607174 | NF2 |
| (3) | |
| Meningioma, SIS-related (3) | PDGFB, SIS |
| Meningococcal disease, susceptibility to (3) | MBL2, MBL, MBP1 |
| Menkes disease, 309400 (3) | ATP7A, MNK, MK, OHS |
| Mental retardation, nonsyndromic, | PRSS12, BSSP3 |
| autosomal recessive, 249500 (3) | |
| Mental retardation, nonsyndromic, | CRBN, MRT2A |
| autosomal recessive, 2A, 607417 (3) | |
| Mental retardation, X-linked, 300425 (3) | NLGN4, KIAA1260, AUTSX2 |
| Mental retardation, X-linked, 300458 (3) | MECP2, RTT, PPMX, MRX16, MRX79 |
| Mental retardation, X-linked 30, 300558 (3) | PAK3, MRX30, MRX47 |
| Mental retardation, X-linked, 34, 300426 (3) | IL1RAPL, MRX34 |
| Mental retardation, X-linked 36, 300430 (3) | ARX, ISSX, PRTS, MRXS1, MRX36, |
| | MRX54 |
| Mental retardation, X-linked (3) | SLC6A8, CRTR |
| Mental retardation, X-linked-44, 300501 (3) | FTSJ1, JM23, SPB1, MRX44, MRX9 |
| Mental retardation, X-linked 45, 300498 (3) | ZNF81, MRX45 |
| Mental retardation, X-linked 54, 300419 (3) | ARX, ISSX, PRTS, MRXS1, MRX36, |
| | MRX54 |
| Mental retardation, X-linked 58, 300218 (3) | TM4SF2, MXS1, A15 |
| Mental retardation, X-linked, 60, 300486 (3) | OPHN1 |
| Mental retardation, X-linked-9, 309549 (3) | FTSJ1, JM23, SPB1, MRX44, MRX9 |
| Mental retardation, X-linked, FRAXE type | FMR2, FRAXE, MRX2 |
| (3) | |
| Mental retardation, X-linked, JARID1C- | SMCX, MRXJ, DXS1272E, XE169, |
| related, 300534 (3) | JARID1C |
| Mental retardation, X-linked nonspecific, | GDI1, RABGD1A, MRX41, MRX48 |
| 309541 (3) | |
| Mental retardation, X-linked nonspecific, 63, | FACL4, ACS4, MRX63 |
| 300387 (3) | |
| Mental retardation, X-linked nonspecific, | RPS6KA3, RSK2, MRX19 |
| type 19 (3) | |
| Mental retardation, X-linked nonspecific, | ARHGEF6, MRX46, COOL2 |
| type 46, 300436 (3) | |
| Mental retardation, X-linked nonsyndromic | AGTR2 |
| (3) | |
| Mental retardation, X-linked nonsyndromic | FGD1, FGDY, AAS |
| (3) | |
| Mental retardation, X-linked nonsyndromic | ZNF41 |
| (3) | |
| Meesmann corneal dystrophy, 122100 (3) | KRT12 |
| Meesmann corneal dystrophy, 122100 (3) | KRT3 |
| Megakaryoblastic leukemia, acute (3) | MKL1, AMKL, MAL |
| Megalencephalic leukoencephalopathy with | MLC1, LVM, VL |
| subcortical cysts, 604004 (3) | |
| Megaloblastic anemia-1, Finnish type, | CUBN, IFCR, MGA1 |
| 261100 (3) | |
| Megaloblastic anemia-1, Norwegian type, | AMN |
| 261100 (3) | |
| Melanoma (3) | CDK4, CMM3 |
| Melanoma and neural system tumor | CDKN2A, MTS1, P16, MLM, CMM2 |
| syndrome, 155755 (3) | |
| Melanoma, cutaneous malignant, 2, 155601 | CDKN2A, MTS1, P16, MLM, CMM2 |
| (3) | |
| Melanoma, cutaneous malignant, | XRCC3 |
| susceptibility to (3) | |
| Melanoma, malignant sporadic (3) | STK11, PJS, LKB1 |
| Melanoma, melignant, somatic (3) | BRAF |
| Meleda disease, 248300 (3) | SLURP1, MDM |
| Melnick-Needles syndrome, 309350 (3) | FLNA, FLN1, ABPX, NHBP, OPD1, |
| | OPD2, FMD, MNS |
| Melorheostosis with osteopoikilosis, 155950 | LEMD3, MAN1 |
| (3) | |
| Memory impairment, susceptibility to (3) | BDNF |
| Meniere disease 156000 (3) ( ) | COCH, DFNA9 |
| Meningioma, 607174 (3) | MN1, MGCR |
| Meningioma, 607174 (3) | PTEN, MMAC1 |
| Meningioma, NF2-related, somatic, 607174 | NF2 |
| (3) | |
| Meningioma, SIS-related (3) | PDGFB, SIS |
| Meningococcal disease, susceptibility to (3) | MBL2, MBL, MBP1 |
| Menkes disease, 309400 (3) | ATP7A, MNK, MK, OHS |
| Mental retardation, nonsyndromic, | PRSS12, BSSP3 |
| autosomal recessive, 249500 (3) | |
| Mental retardation, nonsyndromic, | CRBN, MRT2A |
| autosomal recessive, 2A, 607417 (3) | |
| Mental retardation, X-linked, 300425 (3) | NLGN4, KIAA1260, AUTSX2 |
| Mental retardation, X-linked, 300458 (3) | MECP2, RTT, PPMX, MRX16, MRX79 |
| Mental retardation, X-linked 30, 300558 (3) | PAK3, MRX30, MRX47 |
| Mental retardation, X-linked, 34, 300426 (3) | IL1RAPL, MRX34 |
| Mental retardation, X-linked 36, 300430 (3) | ARX, ISSX, PRTS, MRXS1, MRX36, |
| | MRX54 |
| Mental retardation, X-linked (3) | SLC6A8, CRTR |
| Mental retardation, X-linked-44, 300501 (3) | FTSJ1, JM23, SPB1, MRX44, MRX9 |
| Mental retardation, X-linked 45, 300498 (3) | ZNF81, MRX45 |
| Mental retardation, X-linked 54, 300419 (3) | ARX, ISSX, PRTS, MRXS1, MRX36, |
| | MRX54 |
| Mental retardation, X-linked 58, 300218 (3) | TM4SF2, MXS1, A15 |
| Mental retardation, X-linked, 60, 300486 (3) | OPHN1 |
| Mental retardation, X-linked-9, 309549 (3) | FTSJ1, JM23, SPB1, MRX44, MRX9 |
| Mental retardation, X-linked, FRAXE type | FMR2, FRAXE, MRX2 |
| (3) | |
| Mental retardation, X-linked, JARID1C- | SMCX, MRXJ, DXS1272E, XE169, |
| related, 300534 (3) | JARID1C |
| Mental retardation, X-linked nonspecific, | GDI1, RABGD1A, MRX41, MRX48 |
| 309541 (3) | |
| Mental retardation, X-linked nonspecific, 63, | FACL4, ACS4, MRX63 |
| 300387 (3) | |
| Mental retardation, X-linked nonspecific, | RPS6KA3, RSK2, MRX19 |
| type 19 (3) | |
| Mental retardation, X-linked nonspecific, | ARHGEF6, MRX46, COOL2 |
| type 46, 300436 (3) | |
| Mental retardation, X-linked nonsyndromic | AGTR2 |
| (3) | |
| Mental retardation, X-linked nonsyndromic | FGD1, FGDY, AAS |
| (3) | |
| Mental retardation, X-linked nonsyndromic | ZNF41 |
| (3) | |
| Mental retardation, X-linked nonsyndromic, | DLG3, NEDLG, SAP102, MRX |
| DLGS-related (3) | |
| Mental retardation, X-linked, Snyder- | SMS, SRS, MRSR |
| Robinson type, 309583 (3) | |
| Mental retardation, X-linked, with isolated | SOX3, MRGH |
| growth hormone deficiency, 300123 (3) | |
| Mental retardation, X-linked, with | MECP2, RTT, PPMX, MRX16, MRX79 |
| progressive spasticity, 300279 (3) | |
| Mental retardation, X-linked, with seizures | SLC6A8, CRTR |
| and carrier manifestations, 300397 (3) | |
| Mephenytoin poor metabolizer (3) | CYP2C, CYP2C19 |
| Merkel cell carcinoma, somatic (3) | SDHD, PGL1 |
| Mesangial sclerosis, isolated diffuse, | WT1 |
| 256370 (3) | |
| Mesothelioma (3) | BCL10 |
| Metachromatic leukodystrophy, 250100 (3) | ARSA |
| Metachromatic leukodystrophy due to | PSAP, SAP1 |
| deficiency of SAP-1 (3) | |
| Metaphyseal chondrodysplasia, Murk | PTHR1, PTHR |
| Jansen type, 156400 (3) | |
| Metaphyseal chondrodysplasia, Schmid | COL10A1 |
| type (3) | |
| Metaphyseal dysplasia without | RMRP, RMRPR, CHH |
| hypotrichosis, 250460 (3) | |
| Methemoglobinemia due to cytochrome b5 | CYB5 |
| deficiency (3) | |
| Methemoglobinemias, alpha-(3) | HBA1 |
| Methemoglobinemias, beta-(3) | HBB |
| Methemoglobinemia, type I (3) | DIA1 |
| Methemoglobinemia, type II (3) | DIA1 |
| Methionine adenosyltransferase deficiency, | MAT1A, MATA1, SAMS1 |
| autosomal recessive (3) | |
| Methylcobalamin deficiency, cblG type, | MTR |
| 250940 (3) | |
| Methylmalonate semialdehyde | ALDH6A1, MMSDH |
| dehydrogenase deficiency (3) | |
| Methylmalonic aciduria, mut(0) type, 251000 | MUT, MCM |
| (3) | |
| Methylmalonic aciduria, vitamin B12- | MMAA |
| responsive, 251100 (3) | |
| Methylmalonic aciduria, vitamin B12- | MMAB |
| responsive, due to defect in synthesis of | |
| adenosylcobalamin, cblB complementation | |
| type, 251110 (3) | |
| Mevalonicaciduria (3) | MVK, MVLK |
| MHC class II deficiency, complementation | RFXANK |
| group B, 209920 (3) | |
| Microcephaly, Amish type, 607196 (3) | SLC25A19, DNC, MUP1, MCPHA |
| Microcephaly, autosomal recessive 1, | MCPH1 |
| 251200 (3) | |
| Microcephaly, primary autosomal recessive, | CDK5RAP2, KIAA1633, MCPH3 |
| 3, 604804 (3) | |
| Microcephaly, primary autosomal recessive, | ASPM, MCPH5 |
| 5, 608716 (3) | |
| Microcephaly, primary autosomal recessive, | CEMPJ, CPAP, MCPH6 |
| 6, 608393 (3) | |
| Microcoria-congenital nephrosis syndrome, | LAMB2, LAMS |
| 609049 (3) | |
| Micropenis (3) | LHCGR |
| Microphthalmia, cataracts, and iris | CHX10, HOX10 |
| abnormalities (3) | |
| Microphthalmia, SIX6-related (3) | SIX6 |
| Microphthalmia with associated anomalies | BCOR, KIAA1575, MAA2, ANOP2 |
| 2, 300412 (3) | |
| Migraine, familial hemiplegic, 2, 602481 (3) | ATP1A2, FHM2, MHP2 |
| Migraine, resistance to, 157300 (3) | EDNRA |
| Migraine, susceptibility to, 157300 (3) | ESR1, ESR |
| Migraine without aura, susceptibility to, | TNF, TNFA |
| 157300 (3) | |
| Miller-Dieker lissencephaly, 247200 (3) | YWHAE, MDCR, MDS |
| Mitochondrial complex I deficiency, 252010 | NDUFS1 |
| (3) | |
| Mitochondrial complex I deficiency, 252010 | NDUFS2 |
| (3) | |
| Mitochondrial complex I deficiency, 252010 | NDUFS4, AQDQ |
| (3) | |
| Mitochondrial complex I deficiency, 252010 | NDUFV1, UQOR1 |
| (3) | |
| Mitochondrial complex III deficiency, 124000 | BCS1L, FLNMS, GRACILE |
| (3) | |
| Mitochondrial complex III deficiency, 124000 | UQCRB, UQBP, QPC |
| (3) | |
| Mitochondrial DNA depletion myopathy, | TK2 |
| 251880 (3) | |
| Mitochondrial DNA depletion syndrome, | SUCLA2 |
| 251880 (3) | |
| Mitochondrial DNA-depletion syndrome, | DGUOK, DGK |
| hepatocerebral form, 251880 (3) | |
| Mitochondrial myopathy and sideroblastic | PUS1, MLASA |
| anemia, 600462 (3) | |
| Mitochondrial respiratory chain complex II | SDHA, SDH2, SDHF |
| deficiency, 252011 (3) | |
| Miyoshi myopathy, 254130 (3) | DYSF, LGMD2B |
| MODY5 with nephron agenesis (3) | TCF2, HNF2 |
| MODY5 with non-diabetic renal disease and | TCF2, HNF2 |
| Mullerian aplasia (3) | |
| MODY, one form, 125850 (3) | INS |
| MODY, type I, 125850 (3) | HNF4A, TCF14, MODY1 |
| MODY, type II, 125851 (3) | GCK |
| MODY, type III, 600496 (3) | TCF1, HNF1A, MODY3 |
| MODY, type IV (3) | IPF1 |
| MODY, type V, 604284 (3) | TCF2, HNF2 |
| Mohr-Tranebjaerg syndrome, 304700 (3) | TIMM8A, DFN1, DDP, MTS, DDP1 |
| Molybdenum cofactor deficiency, type A, | MOCS1, MOCOD |
| 252150 (3) | |
| Molybdenum cofactor deficiency, type B, | MOCS2, MPTS |
| 252150 (3) | |
| Molybdenum cofactor deficiency, type C, | GPH, KIAA1385, GEPH |
| 252150 (3) | |
| Monilethrix, 158000 (3) | KRTHB1, HB1 |
| Monilethrix, 158000 (3) | KRTHB6, HB6 |
| Morning glory disc anomaly (3) | PAX6, AN2, MGDA |
| Mowat-Wilson syndrome, 235730 (3) | ZFHX1B, SMADIP1, SIP1 |
| Moyamoya disease 3 (3) | MYMY3 |
| Muckle-Wells syndrome, 191900 (3) | CIAS1, C1orf7, FCU, FCAS |
| Mucoepidermoid salivary gland carcinoma | MAML2, MAM3 |
| (3) | |
| Mucoepidermoid salivary gland carcinoma | MECT1, KIAA0616 |
| (3) | |
| Mucolipidosis IIIA, 252600 (3) | GNPTAB, GNPTA |
| Mucolipidosis IIIC, 252605 (3) | GNPTAG |
| Mucolipidosis IV, 252650 (3) | MCOLN1, ML4 |
| Mucopolysaccharidosis Ih, 607014 (3) | IDUA, IDA |
| Mucopolysaccharidosis Ih/s, 607015 (3) | IDUA, IDA |
| Mucopolysaccharidosis II (3) | IDS, MPS2, SIDS |
| Mucopolysaccharidosis Is, 607016 (3) | IDUA, IDA |
| Mucopolysaccharidosis IVA (3) | GALNS, MPS4A |
| Mucopolysaccharidosis IVB (3) | GLB1 |
| Mucopolysaccharidosis type IIID, 252940 | GNS, G6S |
| (3) | |
| Mucopolysaccharidosis type IX, 601492 (3) | HYAL1 |
| Mucopolysaccharidosis VII (3) | GUSB, MPS7 |
| Muenke syndrome, 602849 (3) | FGFR3, ACH |
| Muir-Torre syndrome, 158320 (3) | MLH1, COCA2, HNPCC2 |
| Muir-Torre syndrome, 158320 (3) | MSH2, COCA1, FCC1, HNPCC1 |
| Mulibrey nanism, 253250 (3) | TRIM37, MUL, KIAA0898 |
| Multiple cutaneous and uterine | FH |
| leiomyomata, 150800 (3) | |
| Multiple endocrine neoplasia I (3) | MEN1 |
| Multiple endocrine neoplasia IIA, 171400 (3) | RET, MEN2A |
| Multiple endocrine neoplasia IIB, 162300 (3) | RET, MEN2A |
| Multiple malignancy syndrome (3) | TP53, P53, LFS1 |
| Multiple myeloma (3) | IRF4, LSIRF |
| Multiple myeloma, resistance to, 254500 (3) | LIG4 |
| Multiple sclerosis, susceptibility to, 126200 | MHC2TA, C2TA |
| (3) | |
| Multiple sclerosis, susceptibility to, 126200 | PTPRC, CD45, LCA |
| (3) | |
| Multiple sulfatase deficiency, 272200 (3) | SUMF1, FGE |
| Muscle-eye-brain disease, 253280 (3) | POMGNT1, MEB |
| Muscle glycogenosis (3) | PHKA1 |
| Muscle hypertrophy (3) | GDF8, MSTN |
| Muscular dystrophy, congenital, 1C (3) | FKRP, MDC1C, LGMD2I |
| Muscular dystrophy, congenital, due to | LAMA2, LAMM |
| partial LAMA2 deficiency, 607855 (3) | |
| Muscular dystrophy, congenital merosin- | LAMA2, LAMM |
| deficient, 607855 (3) | |
| Muscular dystrophy, congenital, type 1D, | LARGE, KIAA0609, MDC1D |
| 608840 (3) | |
| Muscular dystrophy, Fukuyama congenital, | FCMD |
| 253800 (3) | |
| Muscular dystrophy, limb-girdle, type 1A, | TTID, MYOT |
| 159000 (3) | |
| Muscular dystrophy, limb-girdle, type 2A, | CAPN3, CANP3 |
| 253600 (3) | |
| Muscular dystrophy, limb-girdle, type 2B, | DYSF, LGMD2B |
| 253601 (3) | |
| Muscular dystrophy, limb-girdle, type 2C, | SGCG, LGMD2C, DMDA1, SCG3 |
| 253700 (3) | |
| Muscular dystrophy, limb-girdle, type 2D, | SGCA, ADL, DAG2, LGMD2D, DMDA2 |
| 608099 (3) | |
| Muscular dystrophy, limb-girdle, type 2E, | SGCB, LGMD2E |
| 604286 (3) | |
| Muscular dystrophy, limb-girdle, type 2F, | SGCD, SGD, LGMD2F, CMD1L |
| 601287 (3) | |
| Muscular dystrophy, limb-girdle, type 2G, | TCAP, LGMD2G, CMD1N |
| 601954 (3) | |
| Muscular dystrophy, limb-girdle, type 2H, | TRIM32, HT2A, LGMD2H |
| 254110 (3) | |
| Muscular dystrophy, limb-girdle, type 2I, | FKRP, MDC1C, LGMD2I |
| 607155 (3) | |
| Muscular dystrophy, limb-girdle, type 2J, | TTN, CMD1G, TMD, LGMD2J |
| 608807 (3) | |
| Muscular dystrophy, limb-girdle, type 2K, | POMT1 |
| 609308 (3) | |
| Muscular dystrophy, limb-girdle, type 1C, | CAV3, LGMD1C |
| 607801 (3) | |
| Muscular dystrophy, rigid spine, 1, 602771 | SEPN1, SELN, RSMD1 |
| (3) | |
| Muscular dystrophy with epidermolysis | PLEC1, PLTN, EBS1 |
| bullosa simplex, 226670 (3) | |
| Myasthenia, familial infantile, 1, 605809 (3) | CMS1A1, FIM1 |
| Myasthenic syndrome (3) | SCN4A, HYPP, NAC1A |
| Myasthenic syndrome, congenital, | CHRNB1, ACHRB, SCCMS, CMS2A, |
| associated with acetylcholine receptor | CMS1D |
| deficiency, 608931 (3) | |
| Myasthenic syndrome, congenital, | CHRNE, SCCMS, CMS2A, FCCMS, |
| associated with acetylcholine receptor | CMS1E, CMS1D |
| deficiency, 608931 (3) | |
| Myasthenic syndrome, congenital, | RAPSN, CMS1D, CMS1E |
| associated with acetylcholine receptor | |
| deficiency, 608931 (3) | |
| Myasthenic syndrome, congenital, | CHAT, CMS1A2 |
| associated with episodic apnea, 254210 (3) | |
| Myasthenic syndrome, congenital, | RAPSN, CMS1D, CMS1E |
| associated with facial dysmorphism and | |
| acetylcholine receptor deficiency, 608931 (3) | |
| Myasthenic syndrome, fast-channel | CHRNA1, ACHRD, CMS2A, SCCMS, |
| congenital, 608930 (3) | FCCMS |
| Myasthenic syndrome, fast-channel | CHRND, ACHRD, SCCMS, CMS2A, |
| congenital, 608930 (3) | FCCMS |
| Myasthenic syndrome, fast-channel | CHRNE, SCCMS, CMS2A, FCCMS, |
| congenital, 608930 (3) | CMS1E, CMS1D |
| Myasthenic syndrome, slow-channel | CHRNA1, ACHRD, CMS2A, SCCMS, |
| congenital, 601462 (3) | FCCMS |
| Myasthenic syndrome, slow-channel | CHRNB1, ACHRB, SCCMS, CMS2A, |
| congenital, 601462 (3) | CMS1D |
| Myasthenic syndrome, slow-channel | CHRND, ACHRD, SCCMS, CMS2A, |
| congenital, 601462 (3) | FCCMS |
| Myasthenic syndrome, slow-channel | CHRNE, SCCMS, CMS2A, FCCMS, |
| congenital, 601462 (3) | CMS1E, CMS1D |
| Mycobacterial and salmonella infections, | IL12RB1 |
| susceptibility to, 209950 (3) | |
| Mycobacterial infection, atypical, familial | IFNGR1 |
| disseminated, 209950 (3) | |
| Mycobacterial infection, atypical, familial | IFNGR2, IFNGT1, IFGR2 |
| disseminated, 209950 (3) | |
| Mycobacterial infection, atypical, familial | STAT1 |
| disseminated, 209950 (3) | |
| Mycobacterium tuberculosis, suceptibility to | NRAMP1, NRAMP |
| infection by, 607948 (3) | |
| Myelodysplasia syndrome-1 (3) | MDS1 |
| Myelodysplastic syndrome (3) | FACL6, ACS2 |
| Myelodysplastic syndrome, preleukemic (3) | IRF1, MAR |
| Myelofibrosis, idiopathic, 254450 (3) | JAK2 |
| Myelogenous leukemia, acute (3) | FACL6, ACS2 |
| Myelogenous leukemia, acute (3) | IRF1, MAR |
| Myeloid leukemia, acute, M4Eo subtype (3) | CBFB |
| Myeloid malignancy, predisposition to (3) | CSF1R, FMS |
| Myelokathexis, isolated (3) | CXCR4, D2S201E, NPY3R, WHIM |
| Myelomonocytic leukemia, chronic (3) | PDGFRB, PDGFR |
| Myeloperoxidase deficiency, 254600 (3) | MPO |
| Myeloproliferative disorder with eosinophilia, | PDGFRB, PDGFR |
| 131440 (3) | |
| Myoadenylate deaminase deficiency (3) | AMPD1 |
| Myocardial infarction, decreased | F7 |
| susceptibility to (3) | |
| Myocardial infarction susceptibility (3) | APOE, AD2 |
| Myocardial infarction, susceptibility to (3) | ACE, DCP1, ACE1 |
| Myocardial infarction, susceptibility to (3) | ALOX5AP, FLAP |
| Myocardial infarction, susceptibility to (3) | LGALS2 |
| Myocardial infarction, susceptibility to (3) | LTA, TNFB |
| Myocardial infarction, susceptibility to (3) | OLR1, LOX1 |
| Myocardial infarction, susceptibility to (3) | THBD, THRM |
| Myocardial infarction, susceptibility to, | GCLM, GLCLR |
| 608446 (3) | |
| Myocardial infarction, susceptibility to, | TNFSF4, GP34, OX4OL |
| 608446 (3) | |
| Myoclonic epilepsy, juvenile, 1, 254770 (3) | EFHC1, FLJ10466, EJM1 |
| Myoclonic epilepsy, severe, of infancy, | GABRG2, GEFSP3, CAE2, ECA2 |
| 607208 (3) | |
| Myoclonic epilepsy with mental retardation | ARX, ISSX, PRTS, MRXS1, MRX36, |
| and spasticity, 300432 (3) | MRX54 |
| Myoglobinuria/hemolysis due to PGK | PGK1, PGKA |
| deficiency (3) | |
| Myokymia with neonatal epilepsy, 606437 | KCNQ2, EBN1 |
| (3) | |
| Myoneurogastrointestinal | ECGF1 |
| encephalomyopathy syndrome, 603041 (3) | |
| Myopathy, actin, congenital, with cores (3) | ACTA1, ASMA, NEM3, NEM1 |
| Myopathy, actin, congenital, with excess of | ACTA1, ASMA, NEM3, NEM1 |
| thin myofilaments, 161800 (3) | |
| Myopathy, cardioskeletal, desmin-related, | CRYAB, CRYA2, CTPP2 |
| with cataract, 608810 (3) | |
| Myopathy, centronuclear, 160150 (3) | MYF6 |
| Myopathy, congenital (3) | ITGA7 |
| Myopathy, desmin-related, cardioskeletal, | DES, CMD1I |
| 601419 (3) | |
| Myopathy, distal, with anterior tibial onset, | DYSF, LGMD2B |
| 606768 (3) | |
| Myopathy, distal, with decreased caveolin 3 | CAV3, LGMD1C |
| (3) | |
| Myopathy due to CPT II deficiency, 255110 | CPT2 |
| (3) | |
| Myopathy due to phosphoglycerate mutase | PGAM2, PGAMM |
| deficiency (3) | |
| Myopathy, Laing distal, 160500 (3) | MYH7, CMH1, MPD1 |
| Myopathy, myosin storage, 608358 (3) | MYH7, CMH1, MPD1 |
| Myopathy, nemaline, 3, 161800 (3) | ACTA1, ASMA, NEM3, NEM1 |
| Myotilinopathy, 609200 (3) | TTID, MYOT |
| Myotonia congenita, atypical, | SCN4A, HYPP, NAC1A |
| acetazolamide-responsive, 608390 (3) | |
| Myotonia congenita, dominant, 160800 (3) | CLCN1 |
| Myotonia congenita, recessive, 255700 (3) | CLCN1 |
| Myotonia levior, recessive (3) | CLCN1 |
| Myotonic dystrophy, 160900 (3) | DMPK, DM, DMK |
| Myotonic dystrophy, type 2, 602668 (3) | ZNF9, CNBP1, DM2, PROMM |
| Myotubular myopathy, X-linked, 310400 (3) | MTM1, MTMX |
| Myxoid liposarcoma (3) | DDIT3, GADD153, CHOP10 |
| Myxoma, intracardiac, 255960 (3) | PRKAR1A, TSE1, CNC1, CAR |
| N-acetylglutamate synthase deficiency, | NAGS |
| 237310 (3) | |
| Nail-patella syndrome, 161200 (3) | LMX1B, NPS1 |
| Nail-patella syndrome with open-angle | LMX1B, NPS1 |
| glaucoma, 137750 (3) | |
| Nance-Horan syndrome, 302350 (3) | NHS |
| Narcolepsy, 161400 (3) | HCRT, OX |
| Nasopharyngeal carcinoma, 161550 (3) | TP53, P53, LFS1 |
| Nasu-Hakola disease, 221770 (3) | TREM2 |
| Nasu-Hakola disease, 221770 (3) | TYROBP, PLOSL, DAP12 |
| Naxos disease, 601214 (3) | JUP, DP3, PDGB |
| Nemaline myopathy, 161800 (3) | TPM2, TMSB, AMCD1, DA1 |
| Nemaline myopathy 1, autosomal dominant, | TPM3, NEM1 |
| 161800 (3) | |
| Nemaline myopathy 2, autosomal recessive, | NEB, NEM2 |
| 256030 (3) | |
| Nemaline myopathy, Amish type, 605355 | TNNT1, ANM |
| (3) | |
| Neonatal ichthyosis-sclerosing cholangitis | CLDN1, SEMP1 |
| syndrome, 607626 (3) | |
| Nephrogenic syndrome of inappropriate | AVPR2, DIR, DI1, ADHR |
| antidiuresis, 300539 (3) | |
| Nephrolithiasis, type I, 310468 (3) | CLCN5, CLCK2, NPHL2, DENTS |
| Nephrolithiasis, uric acid, susceptibility to, | ZNF365, UAN |
| 605990 (3) | |
| Nephronophthisis 2, infantile, 602088 (3) | INVS, INV, NPHP2, NPH2 |
| Nephronophthisis 4, 606966 (3) | NPHP4, SLSN4 |
| Nephronophthisis, adolescent, 604387 (3) | NPHP3, NPH3 |
| Nephronophthisis, juvenile, 256100 (3) | NPHP1, NPH1, SLSN1 |
| Nephropathy, chronic hypocomplementemic | HF1, CFH, HUS |
| (3) | |
| Nephropathy with pretibial epidermolysis | CD151, PETA3, SFA1 |
| bullosa and deafness, 609057 (3) | |
| Nephrosis-1, congenital, Finnish type, | NPHS1, NPHN |
| 256300 (3) | |
| Nephrotic syndrome, steroid-resistant, | PDCN, NPHS2, SRN1 |
| 600995 (3) | |
| Netherton syndrome, 256500 (3) | SPINK5, LEKTI |
| Neural tube defects, maternal risk of, | MTHFD, MTHFC |
| 601634 (3) | |
| Neuroblastoma, 256700 (3) | NME1, NM23 |
| Neuroblastoma, 256700 (3) | PMX2B, NBPHOX, PHOX2B |
| Neurodegeneration, pantothenate kinase- | PANK2, NBIA1, PKAN, HARP |
| associated, 234200 (3) | |
| Neuroectodermal tumors, supratentorial | PMS2, PMSL2, HNPCC4 |
| primitive, with cafe-au-lait spots, 608623 (3) | |
| Neurofibromatosis, familial spinal, 162210 | NF1, VRNF, WSS, NFNS |
| (3) | |
| Neurofibromatosis-Noonan syndrome, | NF1, VRNF, WSS, NFNS |
| 601321 (3) | |
| Neurofibromatosis, type 1 (3) | NF1, VRNF, WSS, NFNS |
| Neurofibromatosis, type 2, 101000 (3) | NF2 |
| Neurofibromatosis, type 1, with leukemia, | MSH2, COCA1, FCC1, HNPCC1 |
| 162200 (3) | |
| Neurofibrosarcoma (3) | MXI1 |
| Neuropathy, congenital hypomyelinating, 1, | EGR2, KROX20 |
| 605253 (3) | |
| Neuropathy, congenital hypomyelinating, | MPZ, CMT1B, CMTDI3, CHM, DSS |
| 605253 (3) | |
| Neuropathy, distal hereditary motor, 608634 | HSPB1, HSP27, CMT2F |
| (3) | |
| Neuropathy, distal hereditary motor, type II, | HSPB8, H11, E2IG1, DHMN2 |
| 158590 (3) | |
| Neuropathy, hereditary sensory and | SPTLC1, LBC1, SPT1, HSN1, HSAN |
| autonomic, type 1, 162400 (3) | |
| Neuropathy, hereditary sensory and | NGFB, HSAN5 |
| autonomic, type V, 608654 (3) | |
| Neuropathy, hereditary sensory, type II, | HSN2 |
| 201300 (3) | |
| Neuropathy, recurrent, with pressure | PMP22, CMT1A, CMT1E, DSS |
| palsies, 162500 (3) | |
| Neutropenia, alloimmune neonatal (3) | FCGR3A, CD16, IGFR3 |
| Neutropenia, congenital, 202700 (3) | ELA2 |
| Neutropenia, severe congenital, 202700 (3) | GFI1, ZNF163 |
| Neutropenia, severe congenital, X-linked, | WAS, IMD2, THC |
| 300299 (3) | |
| Neutrophil immunodeficiency syndrome, | RAC2 |
| 608203 (3) | |
| Nevo syndrome, 601451 (3) | PLOD, PLOD1 |
| Nevus, epidermal, epidermolytic | KRT10 |
| hyperkeratotic type, 600648 (3) | |
| Newfoundland rod-cone dystrophy, 607476 | RLBP1 |
| (3) | |
| Nicotine addiction, protection from (3) | CYP2A6, CYP2A3, CYP2A, P450C2A |
| Nicotine addiction, susceptibility to, 188890 | CHRNA4, ENFL1 |
| (3) | |
| Nicotine dependence, susceptibility to, | GPR51, GABBR2 |
| 188890 (3) | |
| Niemann-Pick disease, type A, 257200 (3) | SMPD1, NPD |
| Niemann-Pick disease, type B, 607616 (3) | SMPD1, NPD |
| Niemann-Pick disease, type C1, 257220 (3) | NPC1, NPC |
| Niemann-pick disease, type C2, 607625 (3) | NPC2, HE1 |
| Niemann-Pick disease, type D, 257220 (3) | NPC1, NPC |
| Night blindness, congenital stationary (3) | GNAT1 |
| Night blindness, congenital stationary, type | CSNB1, NYX |
| 1, 310500 (3) | |
| Night blindness, congenital stationary, type | PDE6B, PDEB, CSNB3 |
| 3, 163500 (3) | |
| Night blindness, congenital stationary, X- | CACNA1F, CSNB2 |
| linked, type 2, 300071 (3) | |
| Night blindness, congenital stationery, | RHO, RP4, OPN2 |
| rhodopsin-related (3) | |
| Nijmegen breakage syndrome, 251260 (3) | NBS1, NBS |
| Nonaka myopathy, 605820 (3) | GNE, GLCNE, IBM2, DMRV, NM |
| Noncompaction of left ventricular | TAZ, EFE2, BTHS, CMD3A, LVNCX |
| myocardium, isolated, 300183 (3) | |
| Non-Hodgkin lymphoma, somatic, 605027 | CASP10, MCH4, ALPS2 |
| (3) | |
| Nonsmall cell lung cancer (3) | IRF1, MAR |
| Nonsmall cell lung cancer, response to | EGFR |
| tyrosine kinase inhibitor in, 211980 (3) | |
| Nonsmall cell lung cancer, somatic (3) | BRAF |
| Noonan syndrome 1, 163950 (3) | PTPN11, PTP2C, SHP2, NS1 |
| Norrie disease (3) | NDP, ND |
| Norum disease, 245900 (3) | LCAT |
| Norwalk virus infection, resistance to (3) | FUT2, SE |
| Nucleoside phosphorylase deficiency, | NP |
| immunodeficiency due to (3) | |
| Obesity, adrenal insufficiency, and red hair | POMC |
| (3) | |
| Obesity, autosomal dominant, 601665 (3) | MC4R |
| Obesity, hyperphagia, and developmental | AKR1C2, DDH2, DD2, HAKRD |
| delay (3) | |
| Obesity, hyperphagia, and developmental | NTRK2, TRKB |
| delay (3) | |
| Obesity, late-onset, 601665 (3) | AGRP, ART, AGRT |
| Obesity, mild, early-onset, 601665 (3) | NR0B2, SHP |
| Obesity, morbid, with hypogonadism (3) | LEP, OB |
| Obesity, morbid, with hypogonadism (3) | LEPR, OBR |
| Obesity, resistance to (3) | PPARG, PPARG1, PPARG2 |
| Obesity, severe, 601665 (3) | PPARG, PPARG1, PPARG2 |
| Obesity, severe, 601665 (3) | SIM1 |
| Obesity, severe, and type II diabetes, | UCP3 |
| 601665 (3) | |
| Obesity, severe, due to leptin deficiency (3) | LEP, OB |
| Obesity, severe, susceptibility to, 601665 (3) | MC3R |
| Obesity, susceptibility to, 300306 (3) | SLC6A14, OBX |
| Obesity, susceptibility to, 601665 (3) | ADRB2 |
| Obesity, susceptibility to, 601665 (3) | ADRB3 |
| Obesity, susceptibility to, 601665 (3) | CART |
| Obesity, susceptibility to, 601665 (3) | ENPP1, PDNP1, NPPS, M6S1, PCA1 |
| Obesity, susceptibility to, 601665 (3) | GHRL |
| Obesity, susceptibility to, 601665 (3) | UCP1 |
| Obesity, susceptibility to, 601665 (3) | UCP2 |
| Obestiy with impaired prohormone | PCSK1, NEC1, PC1, PC3 |
| processing, 600955 (3) | |
| Obsessive-compulsive disorder 1, 164230 | SLC6A4, HTT, OCD1 |
| (3) | |
| Obsessive-compulsive disorder, protection | BDNF |
| against, 164230 (3) | |
| Obsessive-compulsive disorder, | HTR2A |
| susceptibility to, 164230 (3) | |
| Occipital horn syndrome, 304150 (3) | ATP7A, MNK, MK, OHS |
| Ocular albinism, Nettleship-Falls type (3) | OA1 |
| Oculocutaneous albinism, type II, modifier of | MC1R |
| (3) | |
| Oculocutaneous albinism, type IV, 606574 | MATP, AIM1 |
| (3) | |
| Oculodentodigital dysplasia, 164200 (3) | GJA1, CX43, ODDD, SDTY3, ODOD |
| Oculofaciocardiodental syndrome, 300166 | BCOR, KIAA1575, MAA2, ANOP2 |
| (3) | |
| Oculopharyngeal muscular dystorphy, | PABPN1, PABP2, PAB2 |
| 164300 (3) | |
| Oculopharyngeal muscular dystrophy, | PABPN1, PABP2, PAB2 |
| autosomal recessive, 257950 (3) | |
| Odontohypophosphatasia, 146300 (3) | ALPL, HOPS, TNSALP |
| Oguchi disease-1, 258100 (3) | SAG |
| Oguchi disease-2, 258100 (3) | RHOK, RK, GRK1 |
| Oligodendroglioma, 137800 (3) | PTEN, MMAC1 |
| Oligodontia, 604625 (3) | PAX9 |
| Oligodontia-colorectal cancer syndrome, | AXIN2 |
| 608615 (3) | |
| Omenn syndrome, 603554 (3) | DCLRE1C, ARTEMIS, SCIDA |
| Omenn syndrome, 603554 (3) | RAG1 |
| Omenn syndrome, 603554 (3) | RAG2 |
| Opitz G syndrome, type I, 300000 (3) | MID1, OGS1, BBBG1, FXY, OSX |
| Opremazole poor metabolizer (3) | CYP2C, CYP2C19 |
| Optic atrophy 1, 165500 (3) | OPA1, NTG, NPG |
| Optic atrophy and cataract, 165300 (3) | OPA3, MGA3 |
| Optic nerve coloboma with renal disease, | PAX2 |
| 120330 (3) | |
| Optic nerve hypoplasia/aplasia, 165550 (3) | PAX6, AN2, MGDA |
| Oral-facial-digital syndrome 1, 311200 (3) | OFD1, CXorf5 |
| Ornithine transcarbamylase deficiency, | OTC |
| 311250 (3) | |
| Orofacial cleft 6, 608864 (3) | IRF6, VWS, LPS, PIT, PPS, OFC6 |
| Orolaryngeal cancer, multiple, (3) | CDKN2A, MTS1, P16, MLM, CMM2 |
| Oroticaciduria (3) | UMPS, OPRT |
| Orthostatic intolerance, 604715 (3) | SLC6A2, NAT1, NET1 |
| OSMED syndrome, 215150 (3) | COL11A2, STL3, DFNA13 |
| Osseous heteroplasia, progressive, 166350 | GNAS, GNAS1, GPSA, POH, PHP1B, |
| (3) | PHP1A, AHO |
| Ossification of posterior longitudinal | ENPP1, PDNP1, NPPS, M6S1, PCA1 |
| ligament of spine, 602475 (3) | |
| Osteoarthritis, hand, susceptibility to, | MATN3, EDM5, HOA |
| 607850 (3) | |
| Osteoarthritis of hip, female-specific, | FRZB, FRZB1, SRFP3 |
| susceptibility to, 165720 (3) | |
| Osteoarthritis, susceptibility to, 165720 (3) | ASPN, PLAP1 |
| Osteoarthrosis, 165720 (3) | COL2A1 |
| Osteogenesis imperfecta, 3 clinical forms, | COL1A2 |
| 166200, 166210, 259420 (3) | |
| Osteogenesis imperfecta, type I, 166200 (3) | COL1A1 |
| Osteogenesis imperfecta, type II, 166210 | COL1A1 |
| (3) | |
| Osteogenesis imperfecta, type III, 259420 | COL1A1 |
| (3) | |
| Osteogenesis imperfecta, type IV, 166220 | COL1A1 |
| (3) | |
| Osteolysis, familial expansile, 174810 (3) | TNFRSF11A, RANK, ODFR, OFE |
| Osteolysis, idiopathic, Saudi type, 605156 | MMP2, CLG4A, MONA |
| (3) | |
| Osteopetrosis, autosomal dominant, type I, | LRP5, BMND1, LRP7, LR3, OPPG, |
| 607634 (3) | VBCH2 |
| Osteopetrosis, autosomal dominant, type II, | CLCN7, CLC7, OPTA2 |
| 166600 (3) | |
| Osteopetrosis, autosomal recessive, | OSTM1, GL |
| 259700 (3) | |
| Osteopetrosis, recessive, 259700 (3) | CLCN7, CLC7, OPTA2 |
| Osteopetrosis, recessive, 259700 (3) | TCIRG1, TIRC7, OC116, OPTB1 |
| Osteopoikilosis, 166700 (3) | LEMD3, MAN1 |
| Osteoporosis, 166710 (3) | COL1A1 |
| Osteoporosis, 166710 (3) | LRP5, BMND1, LRP7, LR3, OPPG, |
| | VBCH2 |
| Osteoporosis (3) | CALCA, CALC1 |
| Osteoporosis, hypophosphatemic, (3) | SLC17A2, NPT2 |
| Osteoporosis, idiopathic, 166710 (3) | COL1A2 |
| Osteoporosis, postmenopausal, | CALCR, CRT |
| susceptibility, 166710 (3) | |
| Osteoporosis-pseudoglioma syndrome, | LRP5, BMND1, LRP7, LR3, OPPG, |
| 259770 (3) | VBCH2 |
| Osteoporosis, susceptibility to, 166710 (3) | RIL |
| Osteosarcoma (3) | TP53, P53, LFS1 |
| Osteosarcoma, somatic, 259500 (3) | CHEK2, RAD53, CHK2, CDS1, LFS2 |
| Otopalatodigital syndrome, type I, 311300 | FLNA, FLN1, ABPX, NHBP, OPD1, |
| (3) | OPD2, FMD, MNS |
| Otopalatodigital syndrome, type II, 304120 | FLNA, FLN1, ABPX, NHBP, OPD1, |
| (3) | OPD2, FMD, MNS |
| Ovarian cancer (3) | BRCA1, PSCP |
| Ovarian cancer (3) | MSH2, COCA1, FCC1, HNPCC1 |
| Ovarian cancer, 604370 (3) | PIK3CA |
| Ovarian cancer, endometrial type (3) | MSH6, GTBP, HNPCC5 |
| Ovarian cancer, somatic, (3) | ERBB2, NGL, NEU, HER2 |
| Ovarian carcinoma (3) | CDH1, UVO |
| Ovarian carcinoma (3) | RRAS2, TC21 |
| Ovarian carcinoma, endometrioid type (3) | CTNNB1 |
| Ovarian dysgenesis 1, 233300 (3) | FSHR, ODG1 |
| Ovarian dysgenesis 2, 300510 (3) | BMP15, GDF9B, ODG2 |
| Ovarian hyperstimulation syndrome, | FSHR, ODG1 |
| gestational, 608115 (3) | |
| Ovarian sex cord tumors (3) | FSHR, ODG1 |
| Ovarioleukodystrophy, 603896 (3) | EIF2B2 |
| Ovarioleukodystrophy, 603896 (3) | EIF2B4 |
| Ovarioleukodystrophy, 603896 (3) | EIF2B5, LVWM, CACH, CLE |
| Pachyonychia congenita, Jackson-Lawler | KRT17, PC2, PCHC1 |
| type, 167210 (3) | |
| Pachyonychia congenita, Jackson-Lawler | KRT6B, PC2 |
| type, 167210 (3) | |
| Pachyonychia congenita, Jadassohn- | KRT16 |
| Lewandowsky type, 167200 (3) | |
| Pachyonychia congenita, Jadassohn- | KRT6A |
| Lewandowsky type, 167200 (3) | |
| Paget disease, juvenile, 239000 (3) | TNFRSF11B, OPG, OCIF |
| Paget disease of bone, 602080 (3) | SQSTM1, P62, PDB3 |
| Paget disease of bone, 602080 (3) | TNFRSF11A, RANK, ODFR, OFE |
| Pallidopontonigral degeneration, 168610 (3) | MAPT, MTBT1, DDPAC, MSTD |
| Pallister-Hall syndrome, 146510 (3) | GLI3, PAPA, PAPB, ACLS |
| Palmoplantar keratoderma, | KRT16 |
| nonepidermolytic, 600962 (3) | |
| Palmoplantar verrucous nevus, unilateral, | KRT16 |
| 144200 (3) | |
| Pancreatic agenesis, 260370 (3) | IPF1 |
| Pancreatic cancer, 260350 (3) | ARMET, ARP |
| Pancreatic cancer, 260350 (3) | BRCA2, FANCD1 |
| Pancreatic cancer, 260350 (3) | TP53, P53, LFS1 |
| Pancreatic cancer (3) | MADH4, DPC4, SMAD4, JIP |
| Pancreatic cancer/melanoma syndrome, | CDKN2A, MTS1, P16, MLM, CMM2 |
| 606719 (3) | |
| Pancreatic cancer, somatic (3) | ACVR1B, ACVRLK4, ALK4 |
| Pancreatic cancer, sporadic (3) | STK11, PJS, LKB1 |
| Pancreatic carcinoma, somatic, 260350 (3) | KRAS2, RASK2 |
| Pancreatic carcinoma, somatic (3) | RBBP8, RIM |
| Pancreatitis, hereditary, 167800 (3) | PRSS1, TRY1 |
| Pancreatitis, hereditary, 167800 (3) | SPINK1, PSTI, PCTT, TATI |
| Pancreatitis, idiopathic (3) | CFTR, ABCC7, CF, MRP7 |
| Papillary serous carcinoma of the | BRCA1, PSCP |
| peritoneum (3) | |
| Papillon-Lefevre syndrome, 245000 (3) | CTSC, CPPI, PALS, PLS, HMS |
| Paraganglioma, familial malignant, 168000 | SDHB, SDH1, SDHIP |
| (3) | |
| Paragangliomas, familial central nervous | SDHD, PGL1 |
| system, 168000 (3) | |
| Paragangliomas, familial nonchromaffin, 1, | SDHD, PGL1 |
| with and without deafness, 168000 (3) | |
| Paragangliomas, familial nonchromaffin, 3, | SDHC, PGL3 |
| 605373 (3) | |
| Paraganglioma, sporadic corotid body, | SDHD, PGL1 |
| 168000 (3) | |
| Paramyotonia congenita, 168300 (3) | SCN4A, HYPP, NAC1A |
| Parathyroid adenoma, sporadic (3) | MEN1 |
| Parathyroid adenoma with cystic changes, | HRPT2, C1orf28 |
| 145001 (3) | |
| Parathyroid carcinoma, 608266 (3) | HRPT2, C1orf28 |
| Parietal foramina 1, 168500 (3) | MSX2, CRS2, HOX8 |
| Parietal foramina 2, 168500 (3) | ALX4, PFM2, FPP |
| Parietal foramina with cleidocranial | MSX2, CRS2, HOX8 |
| dysplasia, 168550 (3) | |
| Parkes Weber syndrome, 608355 (3) | RASA1, GAP, CMAVM, PKWS |
| Parkinson disease, 168600 (3) | NR4A2, NURR1, NOT, TINUR |
| Parkinson disease, 168600 (3) | SNCAIP |
| Parkinson disease, 168600 (3) | TBP, SCA17 |
| Parkinson disease 4, autosomal dominant | SNCA, NACP, PARK1, PARK4 |
| Lewy body, 605543 (3) | |
| Parkinson disease 7, autosomal recessive | DJ1, PARK7 |
| early-onset, 606324 (3) | |
| Parkinson disease-8, 607060 (3) | LRRK2, PARK8 |
| Parkinson disease, early onset, 605909 (3) | PINK1, PARK6 |
| Parkinson disease, familial, 168600 (3) | UCHL1, PARK5 |
| Parkinson disease, familial, 168601 (3) | SNCA, NACP, PARK1, PARK4 |
| Parkinson disease, juvenile, type 2, 600116 | PRKN, PARK2, PDJ |
| (3) | |
| Parkinson disease, resistance to, 168600 | DBH |
| (3) | |
| Parkinson disease, susceptibility to, 168600 | NDUFV2 |
| (3) | |
| Paroxysmal nocturnal hemoglobinuria (3) | PIGA |
| Paroxysmal nonkinesigenic dyskinesia, | MR1, TAHCCP2, KIPP1184, BRP17, |
| 118800 (3) | PNKD, FPD1, PDC, DYT8 |
| Partington syndrome, 309510 (3) | ARX, ISSX, PRTS, MRXS1, MRX36, |
| | MRX54 |
| PCWH, 609136 (3) | SOX10, WS4 |
| Pelger-Huet anomaly, 169400 (3) | LBR, PHA |
| Pelizaeus-Merzbacher disease, 312080 (3) | PLP1, PMD |
| Pelizaeus-Merzbacher-like disease, | GJA12, CX47, PMLDAR |
| autosomal recessive, 608804 (3) | |
| Pendred syndrome, 274600 (3) | SLC26A4, PDS, DFNB4 |
| Perineal hypospadias (3) | AR, DHTR, TFM, SBMA, KD, SMAX1 |
| Periodic fever, familial, 142680 (3) | TNFRSF1A, TNFR1, TNFAR, FPF |
| Periodontitis, juvenile, 170650 (3) | CTSC, CPPI, PALS, PLS, HMS |
| Periventricular heterotopia with | ARFGEF2, BIG2 |
| microcephaly, 608097 (3) | |
| Peroxisomal biogenesis disorder, | PEX6, PXAAA1, PAF2 |
| complementation group 4 (3) | |
| Peroxisomal biogenesis disorder, | PEX6, PXAAA1, PAF2 |
| complementation group 6 (3) | |
| Peroxisome biogenesis factor 12 (3) | PEX12 |
| Persistent hyperinsulinemic hypoglycemia of | KCNJ11, BIR, PHHI |
| infancy, 256450 (3) | |
| Persistent Mullerian duct syndrome, type I, | AMH, MIF |
| 261550 (3) | |
| Persistent Mullerian duct syndrome, type II, | AMHR2, AMHR |
| 261550 (3) | |
| Peters anomaly, 603807 (3) | PAX6, AN2, MGDA |
| Peters anomaly, 604229 (3) | CYP1B1, GLC3A |
| Peutz-Jeghers syndrome, 175200 (3) | STK11, PJS, LKB1 |
| Pfeiffer syndrome, 101600 (3) | FGFR1, FLT2, KAL2 |
| Pfeiffer syndrome, 101600 (3) | FGFR2, BEK, CFD1, JWS |
| Phenylketonuria (3) | PAH, PKU1 |
| Phenylketonuria due to dihydropteridine | QDPR, DHPR |
| reductase deficiency (3) | |
| Phenylketonuria due to PTS deficiency (3) | PTS |
| Phenylthiocarbamide tasting, 171200 (3) | TAS2R38, T2R61, PTC |
| Pheochromocytoma, 171300 (3) | SDHD, PGL1 |
| Pheochromocytoma, 171300 (3) | VHL |
| Pheochromocytoma, extraadrenal, and | SDHB, SDH1, SDHIP |
| cervical paraganglioma, 115310 (3) | |
| Phosphoglycerate dehydrogenase | PHGDH |
| deficiency, 601815 (3) | |
| Phosphoribosyl pyrophosphate synthetase- | PRPS1 |
| related gout (3) | |
| Phosphorylase kinase deficiency of liver and | PHKB |
| muscle, autosomal recessive, 261750 (3) | |
| Phosphoserine phosphatase deficiency (3) | PSP |
| Pick disease, 172700 (3) | PSEN1, AD3 |
| Piebaldism (3) | KIT, PBT |
| Pigmentation of hair, skin, and eyes, | MATP, AIM1 |
| variation in (3) | |
| Pigmented adrenocortical disease, primary | PRKAR1A, TSE1, CNC1, CAR |
| isolated, 160980 (3) | |
| Pigmented paravenous chorioretinal | CRB1, RP12 |
| atrophy, 172870 (3) | |
| Pilomatricoma, 132600 (3) | CTNNB1 |
| Pituitary ACTH-secreting adenoma (3) | GNAI2, GNAI2B, GIP |
| Pituitary ACTH secreting adenoma (3) | GNAS, GNAS1, GPSA, POH, PHP1B, |
| | PHP1A, AHO |
| Pituitary adenoma, nonfunctioning (3) | THRA, ERBA1, THRA1 |
| Pituitary anomalies with holoprosencephaly- | GLI2 |
| like features (3) | |
| Pituitary hormone deficiency, combined (3) | POU1F1, PIT1 |
| Pituitary hormone deficiency, combined (3) | PROP1 |
| Pituitary hormone deficiency, combined, | HESX1, RPX |
| HESX1-related, 182230 (3) | |
| Pituitary hormone deficiency, combined, | LHX3 |
| with rigid cervical spine, 262600 (3) | |
| Pituitary tumor, invasive (3) | PRKCA, PKCA |
| Placental abruption (3) | NOS3 |
| Placental steroid sulfatase deficiency (3) | STS, ARSC1, ARSC, SSDD |
| Plasmin inhibitor deficiency (3) | PLI, SERPINF2 |
| Plasminogen Tochigi disease (3) | PLG |
| Platelet-activating factor acetylhydrolase | PLA2G7, PAFAH |
| deficiency (3) | |
| Platelet ADP receptor defect (3) | P2RY12, P2Y12 |
| Platelet disorder, familial, with associated | RUNX1, CBFA2, AML1 |
| myeloid malignancy, 601399 (3) | |
| Platelet glycoprotein IV deficiency, 608404 | CD36 |
| (3) | |
| Pneumonitis, desquamative interstitial, | SFTPC, SFTP2 |
| 263000 (3) | |
| Pneumothorax, primary spontaneous, | FLCN, BHD |
| 173600 (3) | |
| Polycystic kidney and hepatic disease, | FCYT, PKHD1, ARPKD |
| 263200 (3) | |
| Polycystic kidney disease, adult type I, | PKD1 |
| 173900 (3) | |
| Polycystic kidney disease, adult, type II (3) | PKD2, PKD4 |
| Polycystic kidney disease, infantile severe, | PKDTS |
| with tuberous sclerosis (3) | |
| Polycystic liver disease, 174050 (3) | PRKCSH, G19P1, PCLD |
| Polycystic liver disease, 174050 (3) | SEC63 |
| Polycythemia, benign familial, 263400 (3) | VHL |
| Polycythemia vera, 263300 (3) | JAK2 |
| Polydactyly, postaxial, types A1 and B, | GLI3, PAPA, PAPB, ACLS |
| 174200 (3) | |
| Polydactyly, preaxial, type IV, 174700 (3) | GLI3, PAPA, PAPB, ACLS |
| Polymicrogyria, bilateral frontoparietal, | GPR56, TM7XN1, BFPP |
| 606854 (3) | |
| Polyposis, juvenile intestinal, 174900 (3) | BMPR1A, ACVRLK3, ALK3 |
| Polyposis, juvenile intestinal, 174900 (3) | MADH4, DPC4, SMAD4, JIP |
| Popliteal pterygium syndrome, 119500 (3) | IRF6, VWS, LPS, PIT, PPS, OFC6 |
| Porencephaly, 175780 (3) | COL4A1 |
| Porphyria, acute hepatic (3) | ALAD |
| Porphyria, acute intermittent (3) | HMBS, PBGD, UPS |
| Porphyria, acute intermittent, nonerythroid | HMBS, PBGD, UPS |
| variant (3) | |
| Porphyria, congenital erythropoietic, 263700 | UROS |
| (3) | |
| Porphyria cutanea tarda (3) | UROD |
| Porphyria, hepatoerythropoietic (3) | UROD |
| Porphyria variegata, 176200 (3) | HFE, HLA-H, HFE1 |
| Porphyria variegata, 176200 (3) | PPOX |
| PPM-X syndrome, 300055 (3) | MECP2, RTT, PPMX, MRX16, MRX79 |
| Prader-Willi syndrome, 176270 (3) | NDN |
| Prader-Willi syndrome, 176270 (3) | SNRPN |
| Precocious puberty, male, 176410 (3) | LHCGR |
| Preeclampsia/eclampsia 4 (3) | STOX1, PEE4 |
| Preeclampsia, susceptibility to, 189800 (3) | EPHX1 |
| Preeclampsia, susceptibility to (3) | AGT, SERPINA8 |
| Prekallikrein deficiency (3) | KLKB1, KLK3 |
| Premature chromosome condensation with | MCPH1 |
| microcephaly and mental retardation, | |
| 606858 (3) | |
| Premature ovarian failure, 300511 (3) | DIAPH2, DIA, POF2 |
| Premature ovarian failure 3, 608996 (3) | FOXL2, BPES, BPES1, PFRK, POF3 |
| Primary lateral sclerosis, juvenile, 606353 | ALS2, ALSJ, PLSJ, IAHSP |
| (3) | |
| Prion disease with protracted course, | PRNP, PRIP |
| 606688 (3) | |
| Progressive external ophthalmoplegia with | C10orf2, TWINKLE, PEO1, PEO |
| mitochondrial DNA deletions, 157640 (3) | |
| Progressive external ophthalmoplegia with | POLG, POLG1, POLGA, PEO |
| mitochondrial DNA deletions, 157640 (3) | |
| Progressive external ophthalmoplegia with | SLC25A4, ANT1, T1, PEO3 |
| mitochondrial DNA deletions, 157640 (3) | |
| Proguanil poor metabolizer (3) | CYP2C, CYP2C19 |
| Prolactinoma, hyperparathyroidism, | MEN1 |
| carcinoid syndrome (3) | |
| Prolidase deficiency (3) | PEPD |
| Properdin deficiency, X-linked, 312060 (3) | PFC, PFD |
| Propionicacidemia, 606054 (3) | PCCA |
| Propionicacidemia, 606054 (3) | PCCB |
| Prostate cancer 1, 176807, 601518 (3) | RNASEL, RNS4, PRCA1, HPC1 |
| Prostate cancer, 176807 (3) | BRCA2, FANCD1 |
| Prostate cancer, 176807 (3) | PTEN, MMAC1 |
| Prostate cancer (3) | AR, DHTR, TFM, SBMA, KD, SMAX1 |
| Prostate cancer, familial, 176807 (3) | CHEK2, RAD53, CHK2, CDS1, LFS2 |
| Prostate cancer, hereditary, 176807 (3) | MSR1 |
| Prostate cancer, progression and | EPHB2, EPHT3, DRT, ERK |
| metastasis of, 176807 (3) | |
| Prostate cancer, somatic, 176807 (3) | KLF6, COPEB, BCD1, ZF9 |
| Prostate cancer, somatic, 176807 (3) | MAD1L1, TXBP181 |
| Prostate cancer, susceptibility to, 176807 | AR, DHTR, TFM, SBMA, KD, SMAX1 |
| (3) | |
| Prostate cancer, susceptibility to, 176807 | ATBF1 |
| (3) | |
| Prostate cancer, susceptibility to, 176807 | ELAC2, HPC2 |
| (3) | |
| Prostate cancer, susceptibility to, 176807 | MXI1 |
| (3) | |
| Protein S deficiency (3) | PROS1 |
| Proteinuria, low molecular weight, with | CLCN5, CLCK2, NPHL2, DENTS |
| hypercalciuric nephrocalcinosis (3) | |
| Protoporphyria, erythropoietic (3) | FECH, FCE |
| Protoporphyria, erythropoietic, recessive, | FECH, FCE |
| with liver failure (3) | |
| Proud syndrome, 300004 (3) | ARX, ISSX, PRTS, MRXS1, MRX36, |
| | MRX54 |
| Pseudoachondroplasia, 177170 (3) | COMP, EDM1, MED, PSACH |
| Pseudohermaphroditism, male, with | HSD17B3, EDH17B3 |
| gynecomastia, 264300 (3) | |
| Pseudohermaphroditism, male, with Leydig | LHCGR |
| cell hypoplasia (3) | |
| Pseudohypoaldosteronism, type I, 264350 | SCNN1A |
| (3) | |
| Pseudohypoaldosteronism, type I, 264350 | SCNN1B |
| (3) | |
| Pseudohypoaldosteronism, type I, 264350 | SCNN1G, PHA1 |
| (3) | |
| Pseudohypoaldosteronism type I, autosomal | NR3C2, MLR, MCR |
| dominant, 177735 (3) | |
| Pseudohypoaldosteronism type II (3) | WNK4, PRKWNK4, PHA2B |
| Pseudohypoaldosteronism, type IIC, 145260 | WNK1, PRKWNK1, KDP, PHA2C |
| (3) | |
| Pseudohypoparathyroidism, type Ia, 103580 | GNAS, GNAS1, GPSA, POH, PHP1B, |
| (3) | PHP1A, AHO |
| Pseudohypoparathyroidism, type Ib, 603233 | GNAS, GNAS1, GPSA, POH, PHP1B, |
| (3) | PHP1A, AHO |
| Pseudovaginal perineoscrotal hypospadias, | SRD5A2 |
| 264600 (3) | |
| Pseudovitamin D deficiency rickets 1 (3) | CYP27B1, PDDR, VDD1 |
| Pseudoxanthoma elasticum, autosomal | ABCC6, ARA, ABC34, MLP1, PXE |
| dominant, 177850 (3) | |
| Pseudoxanthoma elasticum, autosomal | ABCC6, ARA, ABC34, MLP1, PXE |
| recessive, 264800 (3) | |
| Psoriasis, susceptibility to, 177900 (3) | PSORS6 |
| Psoriatic arthritis, susceptibility to, 607507 | CARD15, NOD2, IBD1, CD, ACUG, |
| (3) | PSORAS1 |
| Pulmonary alveolar proteinosis, 265120 (3) | CSF2RB |
| Pulmonary alveolar proteinosis, 265120 (3) | SFTPC, SFTP2 |
| Pulmonary alveolar proteinosis, congenital, | SFTPB, SFTB3 |
| 265120 (3) | |
| Pulmonary fibrosis, idiopathic, familial, | SFTPC, SFTP2 |
| 178500 (3) | |
| Pulmonary fibrosis, idiopathic, susceptibility | SFTPA1, SFTP1 |
| to, 178500 (3) | |
| Pulmonary hypertension, familial primary, | BMPR2, PPH1 |
| 178600 (3) | |
| Pycnodysostosis, 265800 (3) | CTSK |
| Pyloric stenosis, infantile hypertrophic, | NOS1 |
| susceptibility to, 179010 (3) | |
| Pyogenic sterile arthritis, pyoderma | PSTPIP1, PSTPIP, CD2BP1, PAPAS |
| gangrenosum, and acne, 604416 (3) | |
| Pyropoikilocytosis (3) | SPTA1 |
| Pyruvate carboxylase deficiency, 266150 (3) | PC |
| Pyruvate dehydrogenase deficiency (3) | PDHA1, PHE1A |
| Pyruvate dehydrogenase E1-beta deficiency | PDHB |
| (3) | |
| Rabson-Mendenhall syndrome, 262190 (3) | INSR |
| Radioulnar synostosis with amegakaryocytic | HOXA11, HOX1I |
| thrombocytopenia, 605432 (3) | |
| RAPADILINO syndrome, 266280 (3) | RECQL4, RTS, RECQ4 |
| Rapid progression to AIDS from HIV1 | CX3CR1, GPR13, V28 |
| infection (3) | |
| Rapp-Hodgkin syndrome, 129400 (3) | TP73L, TP63, KET, EEC3, SHFM4, |
| | LMS, RHS |
| Red hair/fair skin (3) | MC1R |
| Refsum disease, 266500 (3) | PEX7, RCDP1 |
| Refsum disease, 266500 (3) | PHYH, PAHX |
| Refsum disease, infantile, 266510 (3) | PEX1, ZWS1 |
| Refsum disease, infantile form, 266510 (3) | PEX26 |
| Refsum disease, infantile form, 266510 (3) | PXMP3, PAF1, PMP35, PEX2 |
| Renal carcinoma, chromophobe, somatic, | FLCN, BHD |
| 144700 (3) | |
| Renal cell carcinoma, 144700 (3) | TRC8, RCA1, HRCA1 |
| Renal cell carcinoma, clear cell, somatic, | OGG1 |
| 144700 (3) | |
| Renal cell carcinoma, papillary, 1, 605074 | PRCC, RCCP1 |
| (3) | |
| Renal cell carcinoma, papillary, 1, 605074 | TFE3 |
| (3) | |
| Renal cell carcinoma, papillary, familial and | MET |
| sporadic, 605074 (3) | |
| Renal cell carcinoma, somatic (3) | VHL |
| Renal glucosuria, 233100 (3) | SLC5A2, SGLT2 |
| Renal hypoplasia, isolated (3) | PAX2 |
| Renal tubular acidosis, distal, 179800, | SLC4A1, AE1, EPB3 |
| 602722 (3) | |
| Renal tubular acidosis, distal, autosomal | ATP6V0A4, ATP6N1B, VPP2, RTA1C, |
| recessive, 602722 (3) | RTADR |
| Renal tubular acidosis-osteopetrosis | CA2 |
| syndrome (3) | |
| Renal tubular acidosis, proximal, with ocular | SLC4A4, NBC1, KNBC, SLC4A5 |
| abnormalities, 604278 (3) | |
| Renal tubular acidosis with deafness, | ATP6B1, VPP3 |
| 267300 (3) | |
| Renal tubular dysgenesis, 267430 (3) | ACE, DCP1, ACE1 |
| Renal tubular dysgenesis, 267430 (3) | AGTR1, AGTR1A, AT2R1 |
| Renal tubular dysgenesis, 267430 (3) | AGT, SERPINA8 |
| Renal tubular dysgenesis, 267430 (3) | REN |
| Renpenning syndrome, 309500 (3) | PQBP1, NPW38, SHS, MRX55, |
| | MRXS3, RENS1, MRXS8 |
| Response to morphine-6-glucuronide (3) | OPRM1 |
| Resting heart rate, 607276 (3) | ADRB1, ADRB1R, RHR |
| Restrictive dermopathy, lethal, 275210 (3) | ZMPSTE24, FACE1, STE24, MADB |
| Retinal degeneration, autosomal recessive, | NRL, D14S46E, RP27 |
| clumped pigment type (3) | |
| Retinal degeneration, autosomal recessive, | PROM1, PROML1, AC133 |
| prominin-related (3) | |
| Retinal degeneration, late-onset, autosomal | C1QTNF5, CTRP5, LORD |
| dominant, 605670 (3) | |
| Retinal dystrophy, early-onset severe (3) | LRAT |
| Retinitis pigmentosa-10, 180105 (3) | IMPDH1 |
| Retinitis pigmentosa-11, 600138 (3) | PRPF31, PRP31 |
| Retinitis pigmentosa-1, 180100 (3) | RP1, ORP1 |
| Retinitis pigmentosa-12, autosomal | CRB1, RP12 |
| recessive, 600105 (3) | |
| Retinitis pigmentosa-13, 600059 (3) | PRPF8, PRPC8, RP13 |
| Retinitis pigmentosa-14, 600132 (3) | TULP1, RP14 |
| Retinitis pigmentosa-17, 600852 (3) | CA4, RP17 |
| Retinitis pigmentosa-18, 601414 (3) | HPRP3, RP18 |
| Retinitis pigmentosa-19, 601718 (3) | ABCA4, ABCR, STGD1, FFM, RP19 |
| Retinitis pigmentosa-20 (3) | RPE65, RP20 |
| Retinitis pigmentosa-2 (3) | RP2 |
| Retinitis pigmentosa-26, 608380 (3) | CERKL |
| Retinitis pigmentosa-27 (3) | NRL, D14S46E, RP27 |
| Retinitis pigmentosa-30, 607921 (3) | FSCN2, RFSN |
| Retinitis pigmentosa-3, 300389 (3) | RPGR, RP3, CRD, RP15, COD1 |
| Retinitis pigmentosa-4, autosomal dominant | RHO, RP4, OPN2 |
| (3) | |
| Retinitis pigmentosa-7, 608133 (3) | RDS, RP7, PRPH2, PRPH, AVMD, |
| | AOFMD |
| Retinitis pigmentosa-9, 180104 (3) | RP9 |
| Retinitis pigmentosa, AR, 268000 (3) | RLBP1 |
| Retinitis pigmentosa, AR, without hearing | USH2A |
| loss, 268000 (3) | |
| Retinitis pigmentosa, autosomal dominant | RGR |
| (3) | |
| Retinitis pigmentosa, autosomal recessive, | CNGB1, CNCG3L, CNCG2 |
| 268000 (3) | |
| Retinitis pigmentosa, autosomal recessive | CNGA1, CNCG1 |
| (3) | |
| Retinitis pigmentosa, autosomal recessive | PDE6A, PDEA |
| (3) | |
| Retinitis pigmentosa, autosomal recessive | PDE6B, PDEB, CSNB3 |
| (3) | |
| Retinitis pigmentosa, autosomal recessive | RGR |
| (3) | |
| Retinitis pigmentosa, autosomal recessive | RHO, RP4, OPN2 |
| (3) | |
| Retinitis pigmentosa, digenic (3) | ROM1, ROSP1 |
| Retinitis pigmentosa, digenic, 608133 (3) | RDS, RP7, PRPH2, PRPH, AVMD, |
| | AOFMD |
| Retinitis pigmentosa, juvenile (3) | AIPL1, LCA4 |
| Retinitis pigmentosa, late onset, 268000 (3) | NR2E3, PNR, ESCS |
| Retinitis pigmentosa, late-onset dominant, | CRX, CORD2, CRD |
| 268000 (3) | |
| Retinitis pigmentosa, MERTK-related, | MERTK |
| 268000 (3) | |
| Retinitis pigmentosa, X-linked with deafness | RPGR, RP3, CRD, RP15, COD1 |
| and sinorespiratory infections, 300455 (3) | |
| Retinitis pigmentosa, X-linked, with | RPGR, RP3, CRD, RP15, COD1 |
| recurrent respiratory infections, 300455 (3) | |
| Retinitis punctata albescens, 136880 (3) | RDS, RP7, PRPH2, PRPH, AVMD, |
| | AOFMD |
| Retinitis punctata albescens, 136880 (3) | RLBP1 |
| Retinoblastoma (3) | RB1 |
| Retinol binding protein, deficiency of (3) | RBP4 |
| Retinoschisis (3) | RS1, XLRS1 |
| Rett syndrome, 312750 (3) | MECP2, RTT, PPMX, MRX16, MRX79 |
| Rett syndrome, atypical, 312750 (3) | CDKL5, STK9 |
| Rett syndrome, preserved speech variant, | MECP2, RTT, PPMX, MRX16, MRX79 |
| 312750 (3) | |
| Rhabdoid predisposition syndrome, familial | SMARCB1, SNF5, INI1, RDT |
| (3) | |
| Rhabdoid tumors (3) | SMARCB1, SNF5, INI1, RDT |
| Rhabdomyosarcoma, 268210 (3) | SLC22A1L, BWSCR1A, IMPT1 |
| Rhabdomyosarcoma, alveolar, 268220 (3) | FOXO1A, FKHR |
| Rhabdomyosarcoma, alveolar, 268220 (3) | PAX3, WS1, HUP2, CDHS |
| Rhabdomyosarcoma, alveolar, 268220 (3) | PAX7 |
| Rheumatoid arthritis, progression of, | IL10, CSIF |
| 180300 (3) | |
| Rheumatoid arthritis, susceptibility to, | MHC2TA, C2TA |
| 180300 (3) | |
| Rheumatoid arthritis, susceptibility to, | NFKBIL1 |
| 180300 (3) | |
| Rheumatoid arthritis, susceptibility to, | PADI4, PADI5, PAD |
| 180300 (3) | |
| Rheumatoid arthritis, susceptibility to, | PTPN8, PEP, PTPN22, LYP |
| 180300 (3) | |
| Rheumatoid arthritis, susceptibility to, | RUNX1, CBFA2, AML1 |
| 180300 (3) | |
| Rheumatoid arthritis, susceptibility to, | SLC22A4, OCTN1 |
| 180300 (3) | |
| Rheumatoid arthritis, systemic juvenile, | MIF |
| susceptibility to, 604302 (3) | |
| Rhizomelic chondrodysplasia punctata, type | PEX7, RCDP1 |
| 1, 215100 (3) | |
| Rhizomelic chondrodysplasia punctata, type | AGPS, ADHAPS |
| 3, 600121 (3) | |
| Rh-mod syndrome (3) | RHAG, RH50A |
| Rh-negative blood type (3) | RHD |
| Rh-null disease, amorph type (3) | RHCE |
| Ribose 5-phosphate isomerase deficiency, | RPIA, RPI |
| 608611 (3) | |
| Rickets due to defect in vitamin D 25- | CYP2R1 |
| hydroxylation, 600081 (3) | |
| Rickets, vitamin D-resistant, type IIA, | VDR |
| 277440 (3) | |
| Rickets, vitamin D-resistant, type IIB, | VDR |
| 277420 (3) | |
| Rieger anomaly (3) | FOXC1, FKHL7, FREAC3 |
| Rieger syndrome, 180500 (3) | PITX2, IDG2, RIEG1, RGS, IGDS2 |
| Ring dermoid of cornea, 180550 (3) | PITX2, IDG2, RIEG1, RGS, IGDS2 |
| Rippling muscle disease, 606072 (3) | CAV3, LGMD1C |
| Roberts syndrome, 268300 (3) | ESCO2 |
| Robinow syndrome, autosomal recessive, | ROR2, BDB1, BDB, NTRKR2 |
| 268310 (3) | |
| Rokitansky-Kuster-Hauser syndrome, | WNT4 |
| 277000 (3) | |
| Rothmund-Thomson syndrome, 268400 (3) | RECQL4, RTS, RECQ4 |
| Roussy-Levy syndrome, 180800 (3) | MPZ, CMT1B, CMTDI3, CHM, DSS |
| Roussy-Levy syndrome, 180800 (3) | PMP22, CMT1A, CMT1E, DSS |
| Rubenstein-Taybi syndrome, 180849 (3) | CREBBP, CBP, RSTS |
| Rubinstein-Taybi syndrome, 180849 (3) | EP300 |
| Saethre-Chotzen syndrome, 101400 (3) | FGFR2, BEK, CFD1, JWS |
| Saethre-Chotzen syndrome, 101400 (3) | TWIST, ACS3, SCS |
| Saethre-Chotzen syndrome with eyelid | TWIST, ACS3, SCS |
| anomalies, 101400 (3) | |
| Salivary adenoma (3) | HMGA2, HMGIC, BABL, LIPO |
| Salla disease, 604369 (3) | SLC17A5, SIASD, SLD |
| Sandhoff disease, infantile, juvenile, and | HEXB |
| adult forms, 268800 (3) | |
| Sanfilippo syndrome, type A, 252900 (3) | SGSH, MPS3A, SFMD |
| Sanfilippo syndrome, type B (3) | NAGLU |
| Sarcoidosis, early-onset, 181000 (3) | CARD15, NOD2, IBD1, CD, ACUG, |
| | PSORAS1 |
| Sarcoidosis, susceptibility to, 181000 (3) | BTNL2 |
| Sarcoidosis, susceptibility to, 181000 (3) | HLA-DR1B |
| Sarcoma, synovial (3) | SSX1, SSRC |
| Sarcoma, synovial (3) | SSX2 |
| SARS, progression of (3) | ACE, DCP1, ACE1 |
| Schimke immunoosseous dysplasia, | SMARCAL1, HARP, SIOD |
| 242900 (3) | |
| Schindler disease, type I, 609241 (3) | NAGA |
| Schindler disease, type III, 609241 (3) | NAGA |
| Schizencephaly, 269160 (3) | EMX2 |
| Schizoaffective disorder, susceptibility to, | DISC1 |
| 181500 (3) | |
| Schizophrenia 5, 603175 (3) | TRAR4 |
| Schizophrenia, chronic (3) | APP, AAA, CVAP, AD1 |
| Schizophrenia, susceptibility to, 181500 (3) | COMT |
| Schizophrenia, susceptibility to, 181500 (3) | DISC1 |
| Schizophrenia, susceptibility to, 181500 (3) | HTR2A |
| Schizophrenia, susceptibility to, 181500 (3) | RTN4R, NOGOR |
| Schizophrenia, susceptibility to, 181500 (3) | SYN2 |
| Schizophrenia, susceptibility to, 181510 (3) | EPN4, EPNR, KIAA0171, SCZD1 |
| Schizophrenia, susceptibility to, 4 600850 | PRODH, PRODH2, SCZD4 |
| (3) | |
| Schwannomatosis, 162091 (3) | NF2 |
| Schwartz-Jampel syndrome, type 1, 255800 | HSPG2, PLC, SJS, SJA, SJS1 |
| (3) | |
| SCI D, autosomal recessive, T-negative/B- | JAK3, JAKL |
| positive type (3) | |
| Sclerosteosis, 269500 (3) | SOST |
| Scurvy (3) | GULOP, GULO |
| Sea-blue histiocyte disease, 269600 (3) | APOE, AD2 |
| Seasonal affective disorder, susceptibility to, | HTR2A |
| 608516 (3) | |
| Sebastian syndrome, 605249 (3) | MYH9, MHA, FTNS, DFNA17 |
| Seckel syndrome 1, 210600 (3) | ATR, FRP1, SCKL |
| Segawa syndrome, recessive (3) | TH, TYH |
| Seizures, afebrile, 604233 (3) | SCN2A1, SCN2A |
| Seizures, benign familial neonatal-infantile, | SCN2A1, SCN2A |
| 607745 (3) | |
| Selective T-cell defect (3) | ZAP70, SRK, STD |
| Self-healing collodion baby, 242300 (3) | TGM1, ICR2, LI1 |
| SEMD, Pakistani type (3) | PAPSS2, ATPSK2 |
| Senior-Loken syndrome-1, 266900 (3) | NPHP1, NPH1, SLSN1 |
| Senior-Loken syndrome 4, 606996 (3) | NPHP4, SLSN4 |
| Senior-Loken syndrome 5, 609254 (3) | IQCB1, NPHP5, KIAA0036 |
| Sensory ataxic neuropathy, dysarthria, and | POLG, POLG1, POLGA, PEO |
| ophthalmoparesis, 157640 (3) | |
| Sepiapterin reductase deficiency (3) | SPR |
| Sepsis, susceptibility to (3) | CASP12, CASP12P1 |
| Septic shock, susceptibility to (3) | TNF, TNFA |
| Septooptic dysplasia, 182230 (3) | HESX1, RPX |
| Sertoli cell-only syndrome, susceptibility to, | USP26 |
| 305700 (3) | |
| Severe combined immunodeficiency, | DCLRE1C, ARTEMIS, SCIDA |
| Athabascan type, 602450 (3) | |
| Severe combined immunodeficiency, B cell- | RAG1 |
| negative, 601457 (3) | |
| Severe combined immunodeficiency, B cell- | RAG2 |
| negative, 601457 (3) | |
| Severe combined immunodeficiency due to | ADA |
| ADA deficiency, 102700 (3) | |
| Severe combined immunodeficiency due to | PTPRC, CD45, LCA |
| PTPRC deficiency (3) | |
| Severe combined immunodeficiency, T-cell | IL7R |
| negative, B-cell/natural killer cell-positive | |
| type, 600802 (3) | |
| Severe combined immunodeficiency, T- | CD3D, T3D |
| negative/B-positive type, 600802 (3) | |
| Severe combined immunodeficiency, X- | IL2RG, SCIDX1, SCIDX, IMD4 |
| linked, 300400 (3) | |
| Sex reversal, XY, with adrenal failure (3) | FTZF1, FTZ1, SF1 |
| Sezary syndrome (3) | BCL10 |
| Shah-Waardenburg syndrome, 277580 (3) | EDN3 |
| Short stature, autosomal dominant, with | GHR |
| normal serum growth hormone binding | |
| protein (3) | |
| Short stature, idiopathic (3) | GHR |
| Short stature, idiopathic familial, 604271 (3) | SHOX, GCFX, SS, PHOG |
| Short stature, idiopathic familial, 604271 (3) | SHOXY |
| Short stature, pituitary and cerebellar | LHX4 |
| defects, and small sella turcica, 606606 (3) | |
| Shprintzen-Goldberg syndrome, 182212 (3) | FBN1, MFS1, WMS |
| Shwachman-Diamond syndrome, 260400 | SBDS, SDS |
| (3) | |
| Sialic acid storage disorder, infantile, | SLC17A5, SIASD, SLD |
| 269920 (3) | |
| Sialidosis, type I, 256550 (3) | NEU1, NEU, SIAL1 |
| Sialidosis, type II, 256550 (3) | NEU1, NEU, SIAL1 |
| Sialuria, 269921 (3) | GNE, GLCNE, IBM2, DMRV, NM |
| Sickle cell anemia (3) | HBB |
| Sick sinus syndrome, 608567 (3) | SCN5A, LQT3, IVF, HB1, SSS1 |
| Silver spastic paraplegia syndrome, 270685 | BSCL2, SPG17 |
| (3) | |
| Simpson-Golabi-Behmel syndrome, type 1, | GPC3, SDYS, SGBS1 |
| 312870 (3) | |
| Sitosterolemia, 210250 (3) | ABCG5 |
| Sitosterolemia, 210250 (3) | ABCG8 |
| Situs ambiguus (3) | NODAL |
| Situs inversus viscerum, 270100 (3) | DNAH11, DNAHC11 |
| Sjogren-Larsson syndrome, 270200 (3) | ALDH3A2, ALDH10, SLS, FALDH |
| Skin fragility-woolly hair syndrome, 607655 | DSP, KPPS2, PPKS2 |
| (3) | |
| Slow acetylation (3) | NAT2, AAC2 |
| Slowed nerve conduction velocity, AD, | ARHGEF10, KIAA0294 |
| 608236 (3) | |
| Small patella syndrome, 147891 (3) | TBX4 |
| SMED Strudwick type, 184250 (3) | COL2A1 |
| Smith-Fineman-Myers syndrome, 309580 | ATRX, XH2, XNP, MRXS3, SHS |
| (3) | |
| Smith-Lemli-Opitz syndrome, 270400 (3) | DHCR7, SLOS |
| Smith-Magenis syndrome, 182290 (3) | RAI1, SMCR, SMS |
| Smith-McCort dysplasia, 607326 (3) | DYM, FLJ90130, DMC, SMC |
| Solitary median maxillary central incisor, | SHH, HPE3, HLP3, SMMCI |
| 147250 (3) | |
| Somatotrophinoma (3) | GNAS, GNAS1, GPSA, POH, PHP1B, |
| | PHP1A, AHO |
| Sorsby fundus dystrophy, 136900 (3) | TIMP3, SFD |
| Sotos syndrome, 117550 (3) | NSD1, ARA267, STO |
| Spastic ataxia, Charlevoix-Saguenay type, | SACS, ARSACS |
| 270550 (3) | |
| Spastic paralysis, infantile onset ascending, | ALS2, ALSJ, PLSJ, IAHSP |
| 607225 (3) | |
| Spastic paraplegia 10, 604187 (3) | KIF5A, NKHC, SPG10 |
| Spastic paraplegia-13, 605280 (3) | HSPD1, SPG13, HSP60 |
| Spastic paraplegia-2, 312920 (3) | PLP1, PMD |
| Spastic paraplegia-3A, 182600 (3) | SPG3A |
| Spastic paraplegia-4, 182601 (3) | SPG4, SPAST |
| Spastic paraplegia-6, 600363 (3) | NIPA1, SPG6 |
| Spastic paraplegia-7, 607259 (3) | PGN, SPG7, CMAR, CAR |
| Specific granule deficiency, 245480 (3) | CEBPE, CRP1 |
| Speech-language disorder-1, 602081 (3) | FOXP2, SPCH1, TNRC10, CAGH44 |
| Spermatogenic failure, susceptibility to (3) | DAZL, DAZH, SPGYLA |
| Spherocytosis-1 (3) | SPTB |
| Spherocytosis-2 (3) | ANK1, SPH2 |
| Spherocytosis, hereditary (3) | SLC4A1, AE1, EPB3 |
| Spherocytosis, hereditary, Japanese type | EPB42 |
| (3) | |
| Spherocytosis, recessive (3) | SPTA1 |
| Spina bifida, 601634 (3) | MTHFD, MTHFC |
| Spina bifida, risk of, 601634, 182940 (3) | MTR |
| Spina bifida, risk of, 601634, 182940 (3) | MTRR |
| Spinal and bulbar muscular atrophy of | AR, DHTR, TFM, SBMA, KD, SMAX1 |
| Kennedy, 313200 (3) | |
| Spinal muscrular atrophy, late-onset, Finkel | VAPB, VAPC, ALS8 |
| type, 182980 (3) | |
| Spinal muscular atrophy-1, 253300 (3) | SMN1, SMA1, SMA2, SMA3, SMA4 |
| Spinal muscular atrophy-2, 253550 (3) | SMN1, SMA1, SMA2, SMA3, SMA4 |
| Spinal muscular atrophy-3, 253400 (3) | SMN1, SMA1, SMA2, SMA3, SMA4 |
| Spinal muscular atrophy-4, 271150 (3) | SMN1, SMA1, SMA2, SMA3, SMA4 |
| Spinal muscular atrophy, distal, type V, | BSCL2, SPG17 |
| 600794 (3) | |
| Spinal muscular atrophy, distal, type V, | GARS, SMAD1, CMT2D |
| 600794 (3) | |
| Spinal muscular atrophy, juvenile (3) | HEXB |
| Spinal muscular atrophy with respiratory | IGHMBP2, SMUBP2, CATF1, SMARD1 |
| distress, 604320 (3) | |
| Spinocerebellar ataxia-10 (3) | ATXN10, SCA10 |
| Spinocerebellar ataxia-1, 164400 (3) | ATXN1, ATX1, SCA1 |
| Spinocerebellar ataxia 12, 604326 (3) | PPP2R2B |
| Spinocerebellar ataxia 14, 605361 (3) | PRKCG, PKCC, PKCG, SCA14 |
| Spinocerebellar ataxia 17, 607136 (3) | TBP, SCA17 |
| Spinocerebellar ataxia-2, 183090 (3) | ATXN2, ATX2, SCA2 |
| Spinocerebellar ataxia 25 (3) | SCA25 |
| Spinocerebellar ataxia-27, 609307 (3) | FGF14, FHF4, SCA27 |
| Spinocerebellar ataxia 4, pure Japanese | PLEKHG4 |
| type, 117210 (3) | |
| Spinocerebellar ataxia-6, 183086 (3) | CACNA1A, CACNL1A4, SCA6 |
| Spinocerebellar ataxia-7, 164500 (3) | ATXN7, SCA7, OPCA3 |
| Spinocerebellar ataxia 8, 608768 (3) | SCA8 |
| Spinocerebellar ataxia, autosomal recessive | TDP1 |
| with axonal neuropathy, 607250 (3) | |
| Split hand/foot malformation, type 3, 600095 | SHFM3, DAC |
| (3) | |
| Split-hand/foot malformation, type 4, 605289 | TP73L, TP63, KET, EEC3, SHFM4, |
| (3) | LMS, RHS |
| Spondylocarpotarsal synostosis syndrome, | FLNB, SCT, AOI |
| 272460 (3) | |
| Spondylocostal dysostosis, autosomal | DLL3, SCDO1 |
| recessive, 1, 277300 (3) | |
| Spondylocostal dysostosis, autosomal | MESP2 |
| recessive 2, 608681 (3) | |
| Spondyloepimetaphyseal dysplasia, 608728 | MATN3, EDM5, HOA |
| (3) | |
| Spondyloepiphyseal dysplasia, Kimberley | AGC1, CSPG1, MSK16, SEDK |
| type, 608361 (3) | |
| Spondyloepiphyseal dysplasia, Omani type, | CHST3, C6ST, C6ST1 |
| 608637 (3) | |
| Spondyloepiphyseal dysplasia tarda, | SEDL, SEDT |
| 313400 (3) | |
| Spondyloepiphyseal dysplasia tarda with | WISP3, PPAC, PPD |
| progressive arthropathy, 208230 (3) | |
| Spondylometaphyseal dysplasia, Japanese | COL10A1 |
| type (3) | |
| Squamous cell carcinoma, burn scar- | TNFRSF6, APT1, FAS, CD95, ALPS1A |
| related, somatic (3) | |
| Squamous cell carcinoma, head and neck, | ING1 |
| 601400 (3) | |
| Squamous cell carcinoma, head and neck, | TNFRSF10B, DR5, TRAILR2 |
| 601400 (3) | |
| Stapes ankylosis syndrome without | NOG, SYM1, SYNS1 |
| symphalangism, 184460 (3) | |
| Stargardt disease-1, 248200 (3) | ABCA4, ABCR, STGD1, FFM, RP19 |
| Stargardt disease 3, 600110 (3) | ELOVL4, ADMD, STGD2, STGD3 |
| Startle disease, autosomal recessive (3) | GLRA1, STHE |
| Startle disease/hyperekplexia, autosomal | GLRA1, STHE |
| dominant, 149400 (3) | |
| STAT1 deficiency, complete (3) | STAT1 |
| Statins, attenuated cholesterol lowering by | HMGCR |
| (3) | |
| Steatocystoma multiplex, 184500 (3) | KRT17, PC2, PCHC1 |
| Stem-cell leukemia/lymphoma syndrome (3) | ZNF198, SCLL, RAMP, FIM |
| Stevens-Johnson syndrome, | HLA-B |
| carbamazepine-induced, susceptibility to, | |
| 608579 (3) | |
| Stickler syndrome, type I, 108300 (3) | COL2A1 |
| Stickler syndrome, type II, 604841 (3) | COL11A1, STL2 |
| Stickler syndrome, type III, 184840 (3) | COL11A2, STL3, DFNA13 |
| Stomach cancer, 137215 (3) | KRAS2, RASK2 |
| Stroke, susceptibility to, 1, 606799 (3) | PDE4D, DPDE3, STRK1 |
| Stroke, susceptibility to, 601367 (3) | ALOX5AP, FLAP |
| Stuve-Wiedemann syndrome/Schwartz- | LIFR, STWS, SWS, SJS2 |
| Jampel type 2 syndrome, 601559 (3) | |
| Subcortical laminal heteropia, X-linked, | DCX, DBCN, LISX |
| 300067 (3) | |
| Subcortical laminar heterotopia (3) | PAFAH1B1, LIS1 |
| Succinic semialdehyde dehydrogenase | SSADH |
| deficiency (3) | |
| Sucrose intolerance (3) | SI |
| Sudden infant death with dysgenesis of the | TSPYL1, TSPYL, SIDDT |
| testes syndrome, 608800 (3) | |
| Sulfite oxidase deficiency, 272300 (3) | SUOX |
| Superoxide dismutase, elevated | SOD3 |
| extracellular (3) | |
| Supranuclear palsy, progressive, 601104 (3) | MAPT, MTBT1, DDPAC, MSTD |
| Supranuclear palsy, progressive atypical, | MAPT, MTBT1, DDPAC, MSTD |
| 260540 (3) | |
| Supravalvar aortic stenosis, 185500 (3) | ELN |
| Surfactant deficiency, neonatal, 267450 (3) | ABCA3, ABC3 |
| Surfactant protein C deficiency (3) | SFTPC, SFTP2 |
| Sutherland-Haan syndrome-like, 300465 (3) | ATRX, XH2, XNP, MRXS3, SHS |
| Sweat chloride elevation without CF (3) | CFTR, ABCC7, CF, MRP7 |
| Symphalangism, proximal, 185800 (3) | NOG, SYM1, SYNS1 |
| Syndactyly, type III, 186100 (3) | GJA1, CX43, ODDD, SDTY3, ODOD |
| Synostoses syndrome, multiple, 1, 186500 | NOG, SYM1, SYNS1 |
| (3) | |
| Synpolydactyly, 3/3′4, associated with | FBLN1 |
| metacarpal and metatarsal synostoses, | |
| 608180 (3) | |
| Synpolydactyly, type II, 186000 (3) | HOXD13, HOX4I, SPD |
| Synpolydactyly with foot anomalies, 186000 | HOXD13, HOX4I, SPD |
| (3) | |
| Systemic lupus erythematosus, | TNFSF6, APT1LG1, FASL |
| susceptibility, 152700 (3) | |
| Systemic lupus erythematosus, | DNASE1, DNL1 |
| susceptibility to, 152700 (3) | |
| Systemic lupus erythematosus, | PTPN8, PEP, PTPN22, LYP |
| susceptibility to, 152700 (3) | |
| Systemic lupus erythematosus, | PDCD1, SLEB2 |
| susceptibility to, 2, 605218, 152700 (3) | |
| Tall stature, susceptibility to (3) | MCM6 |
| Tangier disease, 205400 (3) | ABCA1, ABC1, HDLDT1, TGD |
| Tarsal-carpal coalition syndrome, 186570 | NOG, SYM1, SYNS1 |
| (3) | |
| Tauopathy and respiratory failure (3) | MAPT, MTBT1, DDPAC, MSTD |
| Tay-Sachs disease, 272800 (3) | HEXA, TSD |
| T-cell acute lymphoblastic leukemia (3) | BAX |
| T-cell immunodeficiency, congenital | WHN |
| alopecia, and nail dystrophy (3) | |
| T-cell prolymphocytic leukemia, sporadic (3) | ATM, ATA, AT1 |
| Temperature-sensitive apoptosis, cellular | DAD1 |
| (3) | |
| Tetra-amelia, autosomal recessive, 273395 | WNT3, INT4 |
| (3) | |
| Tetralogy of Fallot, 187500 (3) | JAG1, AGS, AHD |
| Tetralogy of Fallot, 187500 (3) | ZFPM2, FOG2 |
| Tetralogy of Fallot, 187500 (3) | NKX2E, CSX |
| Thalassemia, alpha-(3) | HBA2 |
| Thalassemia-beta, dominant inclusion-body, | HBB |
| 603902 (3) | |
| Thalassemia, delta-(3) | HBD |
| Thalassemia due to Hb Lepore (3) | HBD |
| Thalassemia, Hispanic gamma-delta-beta | LCRB |
| (3) | |
| Thalassemias, alpha-(3) | HBA1 |
| Thalassemias, beta-(3) | HBB |
| Thanatophoric dysplasia, types I and II, | FGFR3, ACH |
| 187600 (3) | |
| Thiamine-responsive megaloblastic anemia | SLC19A2, THTR1 |
| syndrome, 249270 (3) | |
| Thrombocythemia, essential, 187950 (3) | JAK2 |
| Thrombocythemia, essential, 187950 (3) | THPO, MGDF, MPLLG, TPO |
| Thrombocytopenia-2, 188000 (3) | FLJ14813, THC2 |
| Thrombocytopenia, congenital | MPL, TPOR, MPLV |
| amegakaryocytic, 604498 (3) | |
| Thrombocytopenia, X-linked, 313900 (3) | WAS, IMD2, THC |
| Thrombocytopenia, X-linked, intermittent, | WAS, IMD2, THC |
| 313900 (3) | |
| Thromboembolism susceptibility due to | F5 |
| factor V Leiden (3) | |
| Thrombophilia due to factor V Liverpool (3) | F5 |
| Thrombophilia due to heparin cofactor II | HCF2, HC2, SERPIND1 |
| deficiency (3) | |
| Thrombophilia due to HRG deficiency (3) | HRG |
| Thrombophilia due to protein C deficiency | PROC |
| (3) | |
| Thrombophilia due to thrombomodulin | THBD, THRM |
| defect (3) | |
| Thrombophilia, dysfibrinogenemic (3) | FGB |
| Thrombophilia, dysfibrinogenemic (3) | FGG |
| Thrombosis, hyperhomocysteinemic (3) | CBS |
| Thrombotic thrombocytopenic purpura, | ADAMTS13, VWFCP, TTP |
| familial, 274150 (3) | |
| Thrombycytosis, susceptibility to, 187950 | MPL, TPOR, MPLV |
| (3) | |
| Thymine-uraciluria (3) | DPYD, DPD |
| Thyroid adenoma, hyperfunctioning (3) | TSHR |
| Thyroid carcinoma (3) | TP53, P53, LFS1 |
| Thyroid carcinoma, follicular, 188470 (3) | MINPP1, HIPER1 |
| Thyroid carcinoma, follicular, 188470 (3) | PTEN, MMAC1 |
| Thyroid carcinoma, follicular, somatic, | HRAS |
| 188470 (3) | |
| Thyroid carcinoma, papillary, 188550 (3) | GOLGA5, RFG5, PTC5 |
| Thyroid carcinoma, papillary, 188550 (3) | NCOA4, ELE1, PTC3 |
| Thyroid carcinoma, papillary, 188550 (3) | PCM1, PTC4 |
| Thyroid carcinoma, papillary, 188550 (3) | PRKAR1A, TSE1, CNC1, CAR |
| Thyroid carcinoma, papillary, 188550 (3) | TIF1G, RFG7, PTC7 |
| Thyroid carcinoma, papillary, 188550 (3) | TRIM24, TIF1, TIF1A, PTC6 |
| Thyroid hormone organification defect IIA, | TPO, TPX |
| 274500 (3) | |
| Thyroid hormone resistance, 188570 (3) | THRB, ERBA2, THR1 |
| Thyroid hormone resistance, autosomal | THRB, ERBA2, THR1 |
| recessive, 274300 (3) | |
| Thyrotoxic periodic paralysis, susceptibility | CACNA1S, CACNL1A3, CCHL1A3 |
| to, 188580 (3) | |
| Thyrotropin-releasing hormone resistance, | TRHR |
| generalized (3) | |
| Thyroxine-binding globulin deficiency (3) | TBG |
| Tietz syndrome, 103500 (3) | MITF, WS2A |
| Timothy syndrome, 601005 (3) | CACNA1C, CACNL1A1, CCHL1A1, TS |
| Toenail dystrophy, isolated, 607523 (3) | COL7A1 |
| Tolbutamide poor metabolizer (3) | CYP2C9 |
| Total iodide organification defect, 274500 | TPO, TPX |
| (3) | |
| Townes-Brocks branchiootorenal-like | SALL1, HSAL1, TBS |
| syndrome, 107480 (3) | |
| Townes-Brocks syndrome, 107480 (3) | SALL1, HSAL1, TBS |
| Transaldolase deficiency, 606003 (3) | TALDO1 |
| Transcobalamin II deficiency (3) | TCN2, TC2 |
| Transient bullous of the newborn, 131705 | COL7A1 |
| (3) | |
| Transposition of great arteries, dextro- | CFC1, CRYPTIC, HTX2 |
| looped, 217095 (3) | |
| Transposition of the great arteries, dextro- | THRAP2, PROSIT240, TRAP240L, |
| looped, 608808 (3) | KIAA1025 |
| Treacher Collins mandibulofacial | TCOF1, MFD1 |
| dysostosis, 154500 (3) | |
| Tremor, familial essential, 2, 602134 (3) | HS1BP3, FLJ14249, ETM2 |
| Trichodontoosseous syndrome, 190320 (3) | DLX3, TDO |
| Trichorhinophalangeal syndrome, type I, | TRPS1 |
| 190350 (3) | |
| Trichorhinophalangeal syndrome, type III, | TRPS1 |
| 190351 (3) | |
| Trichothiodystrophy (3) | ERCC3, XPB |
| Trichothiodystrophy, 601675 (3) | ERCC2, EM9 |
| Trichothiodystrophy, complementation | TGF2H5, TTDA, TFB5, C6orf175 |
| group A, 601675 (3) | |
| Trichothiodystrophy, nonphotosensitive 1, | TTDN1, C7orf11, ABHS |
| 234050 (3) | |
| Trifunctional protein deficiency, type I (3) | HADHA, MTPA |
| Trifunctional protein deficiency, type II (3) | HADHB |
| Trismus-pseudocomptodactyly syndrome, | MYH8 |
| 158300 (3) | |
| Tropical calcific pancreatitis, 608189 (3) | SPINK1, PSTI, PCTT, TATI |
| Troyer syndrome, 275900 (3) | SPG20 |
| TSC2 angiomyolipomas, renal, modifier of, | IFNG |
| 191100 (3) | |
| Tuberculosis, susceptibility to (3) | IFNGR1 |
| Tuberculosis, susceptibility to, 607948 (3) | IFNG |
| Tuberous sclerosis-1, 191100 (3) | TSC1, LAM |
| Tuberous sclerosis-2, 191100 (3) | TSC2, LAM |
| Turcot syndrome, 276300 (3) | APC, GS, FPC |
| Turcot syndrome with glioblastoma, 276300 | MLH1, COCA2, HNPCC2 |
| (3) | |
| Turcot syndrome with glioblastoma, 276300 | PMS2, PMSL2, HNPCC4 |
| (3) | |
| Twinning, dizygotic, 276400 (3) | FSHR, ODG1 |
| Tyrosinemia, type I (3) | FAH |
| Tyrosinemia, type II (3) | TAT |
| Tyrosinemia, type III (3) | HPD |
| Ullrich congenital muscular dystrophy, | COL6A1, OPLL |
| 254090 (3) | |
| Ullrich congenital muscular dystrophy, | COL6A3 |
| 254090 (3) | |
| Ullrich scleroatonic muscular dystrophy, | COL6A2 |
| 254090 (3) | |
| Ulnar-mammary syndrome, 181450 (3) | TBX3 |
| Unipolar depression, susceptibility to, | TPH2, NTPH |
| 608516 (3) | |
| Unna-Thost disease, nonepidermolytic, | KRT1 |
| 600962 (3) | |
| Urolithiasis, 2,8-dihydroxyadenine (3) | APRT |
| Urolithiasis, hypophosphatemic (3) | SLC17A2, NPT2 |
| Usher syndrome, type 1B (3) | MYO7A, USH1B, DFNB2, DFNA11 |
| Usher syndrome, type 1C, 276904 (3) | USH1C, DFNB18 |
| Usher syndrome, type 1D, 601067 (3) | CDH23, USH1D |
| Usher syndrome, type 1F, 602083 (3) | PCDH15, DFNB23 |
| Usher syndrome, type 1G, 606943 (3) | SANS, USH1G |
| Usher syndrome, type 2A, 276901 (3) | USH2A |
| Usher syndrome, type 3, 276902 (3) | USH3A, USH3 |
| Usher syndrome, type IIC, 605472 (3) | MASS1, VLGR1, KIAA0686, FEB4, |
| | USH2C |
| Uterine leiomyoma (3) | HMGA2, HMGIC, BABL, LIPO |
| UV-induced skin damage, vulnerability to (3) | MC1R |
| van Buchem disease, type 2, 607636 (3) | LRP5, BMND1, LRP7, LR3, OPPG, |
| | VBCH2 |
| van der Woude syndrome, 119300 (3) | IRF6, VWS, LPS, PIT, PPS, OFC6 |
| VATER association with hydrocephalus, | PTEN, MMAC1 |
| 276950 (3) | |
| Velocardiofacial syndrome, 192430 (3) | TBX1, DGS, CTHM, CAFS, TGA, |
| | DORV, VCFS, DGCR |
| Venous malformations, multiple cutaneous | TEK, TIE2, VMCM |
| and mucosal, 600195 (3) | |
| Venous thrombosis, susceptibility to (3) | SERPINA10, ZPI |
| Ventricular fibrillation, idiopathic, 603829 (3) | SCN5A, LQT3, IVF, HB1, SSS1 |
| Ventricular tachycardia, idiopathic, 192605 | GNAI2, GNAI2B, GIP |
| (3) | |
| Ventricular tachycardia, stress-induced | CASQ2 |
| polymorphic, 604772 (3) | |
| Ventricular tachycardia, stress-induced | RYR2, VTSIP |
| polymorphic, 604772 (3) | |
| Vertical talus, congenital, 192950 (3) | HOXD10, HOX4D |
| Viral infections, recurrent (3) | FCGR3A, CD16, IGFR3 |
| Viral infection, susceptibility to (3) | OAS1, OIAS |
| Virilization, maternal and fetal, from | CYP19A1, CYP19, ARO |
| placental aromatase deficiency (3) | |
| Vitamin K-dependent clotting factors, | VKORC1, VKOR, VKCFD2, FLJ00289 |
| combined deficiency of, 2, 607473 (3) | |
| Vitamin K-dependent coagulation defect, | GGCX |
| 277450 (3) | |
| Vitelliform macular dystrophy, adult-onset, | VMD2 |
| 608161 (3) | |
| VLCAD deficiency, 201475 (3) | ACADVL, VLCAD |
| Vohwinkel syndrome, 124500 (3) | GJB2, CX26, DFNB1, PPK, DFNA3, |
| | KID, HID |
| Vohwinkel syndrome with ichthyosis, | LOR |
| 604117 (3) | |
| von Hippel-Lindau disease, modification of, | CCND1, PRAD1, BCL1 |
| 193300 (3) | |
| von Hippel-Lindau syndrome, 193300 (3) | VHL |
| von Willebrand disease (3) | VWF, F8VWF |
| Waardenburg-Shah syndrome, 277580 (3) | EDNRB, HSCR2, ABCDS |
| Waardenburg-Shah syndrome, 277580 (3) | SOX10, WS4 |
| Waardenburg syndrome/albinism, digenic, | TYR |
| 103470 (3) | |
| Waardenburg syndrome/ocular albinism, | MITF, WS2A |
| digenic, 103470 (3) | |
| Waardenburg syndrome, type I, 193500 (3) | PAX3, WS1, HUP2, CDHS |
| Waardenburg syndrome, type IIA, 193510 | MITF, WS2A |
| (3) | |
| Waardenburg syndrome, type III, 148820 (3) | PAX3, WS1, HUP2, CDHS |
| Waardenburg syndrome, typ IID, 608890 (3) | SNAI2, SLUG, WS2D |
| Wagner syndrome, 143200 (3) | COL2A1 |
| WAGR syndrome, 194072 (3) | WT1 |
| Walker-Warburg syndrome, 236670 (3) | FCMD |
| Walker-Warburg syndrome, 236670 (3) | POMT1 |
| Warburg micro syndrome 1, 600118 (3) | RAB3GAP, WARBM1, P130 |
| Warfarin resistance, 122700 (3) | VKORC1, VKOR, VKCFD2, FLJ00289 |
| Warfarin sensitivity, 122700 (3) | CYP2C9 |
| Warfarin sensitivity (3) | F9, HEMB |
| Watson syndrome, 193520 (3) | NF1, VRNF, WSS, NFNS |
| Weaver syndrome, 277590 (3) | NSD1, ARA267, STO |
| Wegener-like granulomatosis (3) | TAP2, ABCB3, PSF2, RING11 |
| Weill-Marchesani syndrome, dominant, | FBN1, MFS1, WMS |
| 608328 (3) | |
| Weill-Marchesani syndrome, recessive, | ADAMTS10, WMS |
| 277600 (3) | |
| Weissenbacher-Zweymuller syndrome, | COL11A2, STL3, DFNA13 |
| 277610 (3) | |
| Werner syndrome, 277700 (3) | RECQL2, RECQ3, WRN |
| Wernicke-Korsakoff syndrome, susceptibility | TKT |
| to, 277730 (3) | |
| Weyers acrodental dysostosis, 193530 (3) | EVC |
| WHIM syndrome, 193670 (3) | CXCR4, D2S201E, NPY3R, WHIM |
| White sponge nevus, 193900 (3) | KRT13 |
| White sponge nevus, 193900 (3) | KRT4, CYK4 |
| Williams-Beuren syndrome, 194050 (3) | ELN |
| Wilms tumor, 194070 (3) | BRCA2, FANCD1 |
| Wilms tumor, somatic, 194070 (3) | GPC3, SDYS, SGBS1 |
| Wilms tumor susceptibility-5, 601583 (3) | POU6F2, WTSL, WT5 |
| Wilms tumor, type 1, 194070 (3) | WT1 |
| Wilson disease, 277900 (3) | ATP7B, WND |
| Wiskott-Aldrich syndrome, 301000 (3) | WAS, IMD2, THC |
| Witkop syndrome, 189500 (3) | MSX1, HOX7, HYD1, OFC5 |
| Wolcott-Rallison syndrome, 226980 (3) | EIF2AK3, PEK, PERK, WRS |
| Wolff-Parkinson-White syndrome, 194200 | PRKAG2, WPWS |
| (3) | |
| Wolfram syndrome, 222300 (3) | WFS1, WFRS, WFS, DFNA6 |
| Wolman disease (3) | LIPA |
| Xanthinuria, type I, 278300 (3) | XDH |
| Xeroderma pigmentosum, group A (3) | XPA |
| Xeroderma pigmentosum, group B (3) | ERCC3, XPB |
| Xeroderma pigmentosum, group C (3) | XPC, XPCC |
| Xeroderma pigmentosum, group D, 278730 | ERCC2, EM9 |
| (3) | |
| Xeroderma pigmentosum, group E, DDB- | DDB2 |
| negative subtype, 278740 (3) | |
| Xeroderma pigmentosum, group F, 278760 | ERCC4, XPF |
| (3) | |
| Xeroderma pigmentosum, group G, 278780 | ERCC5, XPG |
| (3) | |
| Xeroderma pigmentosum, variant type, | POLH, XPV |
| 278750 (3) | |
| X-inactivation, familial skewed, 300087 (3) | XIC, XCE, XIST, SXI1 |
| XLA and isolated growth hormone | BTK, AGMX1, IMD1, XLA, AT |
| deficiency, 307200 (3) | |
| Yellow nail syndrome, 153300 (3) | FOXC2, FKHL14, MFH1 |
| Yemenite deaf-blind hypopigmentation | SOX10, WS4 |
| syndrome, 601706 (3) | |
| Zellweger syndrome-1, 214100 (3) | PEX1, ZWS1 |
| Zellweger syndrome, 214100 (3) | PEX10, NALD |
| Zellweger syndrome, 214100 (3) | PEX13, ZWS, NALD |
| Zellweger syndrome, 214100 (3) | PEX14 |
| Zellweger syndrome, 214100 (3) | PEX26 |
| Zellweger syndrome, 214100 (3) | PXF, HK33, D1S2223E, PEX19 |
| Zellweger syndrome, 214100 (3) | PXR1, PEX5, PTS1R |
| Zellweger syndrome-2 (3) | ABCD3, PXMP1, PMP70 |
| Zellweger syndrome-3 (3) | PXMP3, PAF1, PMP35, PEX2 |
| Zellweger syndrome, complementation | PEX16 |
| group 9 (3) | |
| Zellweger syndrome, complementation | PEX3 |
| group G, 214100 (3) | |
| Zlotogora-Ogur syndrome, 225000 (3) | HVEC, PVRL1, PVRR1, PRR1 |

TABLE C

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon Receptor | HSPB1; EP300; FASN; TGM2; RXRA; MAPK1; NQO1; |
| Signaling | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| Signaling | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated | IRAK1; MYD88; TRAF6; PPARA; RXRA; ABCA1; |
| Inhibition | MAPK8; ALDH1A1; GSTP1; MAPK9; ABCB1; TRAF2; |
| of RXR Function | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism, | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

Examples of proteins associated with Parkinson's disease include but are not limited to t-synuclein, DJ-1, LRRK2, PINK1, Parkin, UCHL1, Synphilin-1, and NURR1.

Examples of addiction-related proteins include ABAT (4-aminobutyrate aminotransferase); ACN9 (ACN9 homolog (S. cerevisae)); ADCYAP1 (Adenylate cyclase activating polypeptide 1); ADHlB (Alcohol dehydrogenase IB (class I), beta polypeptide); ADH1C (Alcohol dehydrogenase IC (class I), gamma polypeptide); ADH4 (Alcohol dehydrogenase 4); ADH7 (Alcohol dehydrogenase 7 (class IV), mu or sigma polypeptide); ADORA1 (Adenosine A1 receptor); ADRA1A (Adrenergic, alpha-1A-, receptor); ALDH2 (Aldehyde dehydrogenase 2 family); ANKK1 (Ankyrin repeat, TaqI A1 allele); ARC (Activity-regulated cytoskeleton-associated protein); ATF2 (Corticotrophin-releasing factor); AVPR1A (Arginine vasopressin receptor 1A); BDNF (Brain-derived neurotrophic factor); BMAL1 (Aryl hydrocarbon receptor nuclear translocator-like); CDK5 (Cyclin-dependent kinase 5); CHRM2 (Cholinergic receptor, muscarinic 2); CHRNA3 (Cholinergic receptor, nicotinic, alpha 3); CHRNA4 (Cholinergic receptor, nicotinic, alpha 4); CHRNA5 (Cholinergic receptor, nicotinic, alpha 5); CHRNA7 (Cholinergic receptor, nicotinic, alpha 7); CHRNB2 (Cholinergic receptor, nicotinic, beta 2); CLOCK (Clock homolog (mouse)); CNR1 (Cannabinoid receptor 1); CNR2 (Cannabinoid receptor type 2); COMT (Catechol-O-methyltransferase); CREB1 (cAMP Responsive element binding protein 1); CREB2 (Activating transcription factor 2); CRHR1 (Corticotropin releasing hormone receptor 1); CRY1 (Cryptochrome 1); CSNK1E (Casein kinase 1, epsilon); CSPG5 (Chondroitin sulfate proteoglycan 5); CTNNB1 (Catenin (cadherin-associated protein), beta 1, 88 kDa); DBI (Diazepam binding inhibitor); DDN (Dendrin); DRD1 (Dopamine receptor D1); DRD2 (Dopamine receptor D2); DRD3 (Dopamine receptor D3); DRD4 (Dopamine receptor D4); EGR1 (Early growth response 1); ELTD1 (EGF, latrophilin and seven transmembrane domain containing 1); FAAH (Fatty acid amide hydrolase); FOSB (FBJ murine osteosarcoma viral oncogene homolog); FOSB (FBJ murine osteosarcoma viral oncogene homolog B); GABBR2 (Gamma-aminobutyric acid (GABA) B receptor, 2); GABRA2 (Gamma-aminobutyric acid (GABA) A receptor, alpha 2); GABRA4 (Gamma-aminobutyric acid (GABA) A receptor, alpha 4); GABRA6 (Gamma-aminobutyric acid (GABA) A receptor, alpha 6); GABRB3 (Gamma-aminobutyric acid (GABA) A receptor, alpha 3); GABRE (Gamma-aminobutyric acid (GABA) A receptor, epsilon); GABRG1 (Gamma-aminobutyric acid (GABA) A receptor, gamma 1); GAD1 (Glutamate decarboxylase 1); GAD2 (Glutamate decarboxylase 2); GAL (Galanin prepropeptide); GDNF (Glial cell derived neurotrophic factor); GRIA1 (Glutamate receptor, ionotropic, AMPA 1); GRIA2 (Glutamate receptor, ionotropic, AMPA 2); GRIN1 (Glutamate receptor, ionotropic, N-methyl D-aspartate 1); GRIN2A (Glutamate receptor, ionotropic, N-methyl D-aspartate 2A); GRM2 (Glutamate receptor, metabotropic 2, mGluR2); GRM5 (Metabotropic glutamate receptor 5); GRM6 (Glutamate receptor, metabotropic 6); GRM8 (Glutamate receptor, metabotropic 8); HTR1B (5-Hydroxytryptamine (serotonin) receptor IB); HTR3A (5-Hydroxytryptamine (serotonin) receptor 3A); IL1 (Interleukin 1); IL15 (Interleukin 15); ILIA (Interleukin 1 alpha); IL1B (Interleukin 1 beta); KCNMA1 (Potassium large conductance calcium-activated channel, subfamily M, alpha member 1); LGALS1 (lectin galactoside-binding soluble 1); MAOA (Monoamine oxidase A); MAOB (Monoamine oxidase B); MAPK1 (Mitogen-activated protein kinase 1); MAPK3 (Mitogen-activated protein kinase 3); MBP (Myelin basic protein); MC2R (Melanocortin receptor type 2); MGLL (Monoglyceride lipase); MOBP (Myelin-associated oligodendrocyte basic protein); NPY (Neuropeptide Y); NR4A1 (Nuclear receptor subfamily 4, group A, member 1); NR4A2 (Nuclear receptor subfamily 4, group A, member 2); NRXN1 (Neurexin 1); NRXN3 (Neurexin 3); NTRK2 (Neurotrophic tyrosine kinase, receptor, type 2); NTRK2 (Tyrosine kinase B neurotrophin receptor); OPRD1 (delta-Opioid receptor); OPRK1 (kappa-Opioid receptor); OPRM1 (mu-Opioid receptor); PDYN (Dynorphin); PENK (Enkephalin); PER2 (Period homolog 2 (Drosophila)); PKNOX2 (PBX/knotted 1 homeobox 2); PLP1 (Proteolipid protein 1); POMC (Proopiomelanocortin); PRKCE (Protein kinase C, epsilon); PROKR2 (Prokineticin receptor 2); RGS9 (Regulator of G-protein signaling 9); RIMS2 (Regulating synaptic membrane exocytosis 2); SCN9A (sodium channel voltage-gated type IX alpha subunit); SLC17A6 (Solute carrier family 17 (sodium-dependent inorganic phosphate cotransporter), member 6); SLC17A7 (Solute carrier family 17 (sodium-dependent inorganic phosphate cotransporter), member 7); SLC1A2 (Solute carrier family 1 (glial high affinity glutamate transporter), member 2); SLC1A3 (Solute carrier family 1 (glial high affinity glutamate transporter), member 3); SLC29A1 (solute carrier family 29 (nucleoside transporters), member 1); SLC4A7 (Solute carrier family 4, sodium bicarbonate cotransporter, member 7); SLC6A3 (Solute carrier family 6 (neurotransmitter transporter, dopamine), member 3); SLC6A4 (Solute carrier family 6 (neurotransmitter transporter, serotonin), member 4); SNCA (Synuclein, alpha (non A4 component of amyloid precursor)); TFAP2B (Transcription factor AP-2 beta); and TRPV1 (Transient receptor potential cation channel, subfamilyV, member 1).

Examples of inflammation-related proteins include the monocyte chemoattractant protein-1 (MCP1) encoded by the Ccr2 gene, the C-C chemokine receptor type 5 (CCR5) encoded by the Ccr5 gene, the IgG receptor IIB (FCGR2b, also termed CD32) encoded by the Fcgr2b gene, the Fe epsilon R1g (FCER1g) protein encoded by the Fcerlg gene, the forkhead box N1 transcription factor (FOXN1) encoded by the FOXN1 gene, Interferon-gamma (IFN-γ) encoded by the IFNg gene, interleukin 4 (IL-4) encoded by the IL-4 gene, perforin-1 encoded by the PRF-1 gene, the cyclooxygenase 1 protein (COX1) encoded by the COX1 gene, the cyclooxygenase 2 protein (COX2) encoded by the COX2 gene, the T-box transcription factor (TBX21) protein encoded by the TBX21 gene, the SH2-B PH domain containing signaling mediator 1 protein (SH2BPSM1) encoded by the SH2B1 gene (also termed SH2BPSM1), the fibroblast growth factor receptor 2 (FGFR2) protein encoded by the FGFR2 gene, the solute carrier family 22 member 1 (SLC22A1) protein encoded by the OCT1 gene (also termed SLC22A1), the peroxisome proliferator-activated receptor alpha protein (PPAR-alpha, also termed the nuclear receptor subfamily 1, group C, member 1; NR1C1) encoded by the PPARA gene, the phosphatase and tensin homolog protein (PTEN) encoded by the PTEN gene, interleukin 1 alpha (IL-1 a) encoded by the IL-A gene, interleukin 1 beta (IL-10) encoded by the IL-lB gene, interleukin 6 (IL-6) encoded by the IL-6 gene, interleukin 10 (IL-10) encoded by the IL-10 gene, interleukin 12 alpha (IL-12a) encoded by the IL-12A gene, interleukin 12 beta (IL-12β) encoded by the IL-12B gene, interleukin 13 (IL-13) encoded by the IL-13 gene, interleukin 17A (IL-17A, also termed CTLA8) encoded by the IL-17A gene, interleukin 17B (IL-17B) encoded by the IL-17B gene, interleukin 17C (IL-170) encoded by the IL-17C gene interleukin 17D (IL-17D) encoded by the IL-17D gene interleukin 17F (IL-17F) encoded by the IL-17F gene, interleukin 23 (IL-23) encoded by the IL-23 gene, the chemokine (C-X₃-C motif) receptor 1 protein (CX3CR1) encoded by the CX3CR1 gene, the chemokine (C-X₃-C motif) ligand 1 protein (CX3CL1) encoded by the CX3CL1 gene, the recombination activating gene 1 protein (RAG1) encoded by the RAG1 gene, the recombination activating gene 2 protein (RAG2) encoded by the RAG2 gene, the protein kinase, DNA-activated, catalytic polypeptide 1 (PRKDC) encoded by the PRKDC (DNAPK) gene, the protein tyrosine phosphatase non-receptor type 22 protein (PTPN22) encoded by the PTPN22 gene, tumor necrosis factor alpha (TNFα) encoded by the TNFA gene, the nucleotide-binding oligomerization domain containing 2 protein (NOD2) encoded by the NOD2 gene (also termed CARD15), or the cytotoxic T-lymphocyte antigen 4 protein (CTLA4, also termed CD152) encoded by the CTLA4 gene.

Examples of cardiovascular diseases associated protein include IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type N collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), IL1A (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein IIIa, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor uperfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type N collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOAS (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOXS (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDESA (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), URI (interleukin 1 receptor, type I), AR (androgen receptor), CYP1A1 (cytochrome P4SO, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN1 (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X₃-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-1), INSR (insulin receptor), TNFRSF1B (tumor necrosis factor receptor superfamily, member IB), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS 1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calciwn exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Racl)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COLIA2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGCIA (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PRO51 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-O-methyltransferase), SlOOB (S100B calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (8321 placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase IC (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinylleukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity IIIa, receptor (CD16a)), LEPR (leptin receptor), ENG (endoglin), GPX1 (glutathione peroxidase 1), GOT2 (glutamic-oxaloacetic transaminase 2, mitochondrial (aspmiate aminotransferase 2)), HRH1 (histamine receptor H1), NR1I2 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDX5 (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocmticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type 1), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S 100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositidc-3-kinasc, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PAD14 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha IC subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1 (or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine NI-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS 1 (LIM and senescent cell antigen-like domains 1), RHOB (ras homolog gene family, member B), LY96 (lymphocyte antigen 96), FOXO1 (forkhead box O1), PNPLA2 (patatin-like phospholipase domain containing 2), TRH (thyrotropin-releasing hormone), GJC1 (gap junction protein, gamma 1, 45 kDa), SLC17AS (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cellleukemia homeobox 4), NUPR1 (nuclear protein, transcriptional regulator, 1), 15-Sep (15 kDa selenoprotein), CILP2 (cartilage intermediate layer protein 2), TERC (telomerase RNA component), GGT2 (gamma-glutamyltransferase 2), MT-001 (mitochondrially encoded cytochrome c oxidase I), and UOX (urate oxidase, pseudogene).

Examples of Alzheimer's disease associated proteins include the very low density lipoprotein receptor protein (VLDLR) encoded by the VLDLR gene, the ubiquitin-like modifier activating enzyme 1 (UBA1) encoded by the UBA1 gene, the NEDD8-activating enzyme E1 catalytic subunit protein (UBE1C) encoded by the UBA3 gene, the aquaporin 1 protein (AQP1) encoded by the AQP1 gene, the ubiquitin carboxyl-terminal esterase L protein (UCHL1) encoded by the UCHL1 gene, the ubiquitin carboxyl-terminal hydrolase isozyme L3 protein (UCHL3) encoded by the UCHL3 gene, the ubiquitin B protein (UBB) encoded by the UBB gene, the microtubule-associated protein tau (MAPT) encoded by the MAPT gene, the protein tyrosine phosphatase receptor type A protein (PTPRA) encoded by the PTPRA gene, the phosphatidylinositol binding clathrin assembly protein (PICALM) encoded by the PICALM gene, the clusterin protein (also known as apoplipoprotein J) encoded by the CLU gene, the presenilin 1 protein encoded by the PSEN1 gene, the presenilin 2 protein encoded by the PSEN2 gene, the sortilin-related receptor L (DLR class) A repeats-containing protein (SORL1) protein encoded by the SORL1 gene, the amyloid precursor protein (APP) encoded by the APP gene, the Apolipoprotein E precursor (APOE) encoded by the APOE gene, or the brain-derived neurotrophic factor (BDNF) encoded by the BDNF gene, or combinations thereof.

Examples of proteins associated Autism Spectrum Disorder include the benzodiazapine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, the fragile X mental retardation autosomal homolog 2 protein (FXR2) encoded by the FXR2 gene, the MAM domain containing glycosylphosphatidylinositol anchor 2 protein (MDGA2) encoded by the MDGA2 gene, the methyl CpG binding protein 2 (MECP2) encoded by the MECP2 gene, the metabotropic glutamate receptor 5 (MGLUR5) encoded by the MGLUR5-1 gene (also termed GRM5), the neurexin 1 protein encoded by the NRXN1 gene, or the semaphorin-5A protein (SEMA5A) encoded by the SEMA5A gene.

Examples of proteins associated Macular Degeneration include the ATP-binding cassette, sub-family A (ABC1) member 4 protein (ABCA4) encoded by the ABCR gene, the apolipoprotein E protein (APOE) encoded by the APOE gene, the chemokine (C-C motif) Ligand 2 protein (CCL2) encoded by the CCL2 gene, the chemokine (C-C motif) receptor 2 protein (CCR2) encoded by the CCR2 gene, the ceruloplasmin protein (CP) encoded by the CP gene, the cathepsin D protein (CTSD) encoded by the CTSD gene, or the metalloproteinase inhibitor 3 protein (TIMP3) encoded by the TIMP3 gene.

Examples of proteins associated Schizophrenia include NRG1, ErbB4, CPLX1, TPH1, TPH2, NRXN1, GSK3A, BDNF, DISC1, GSK3B, and combinations thereof.

Examples of proteins involved in tumor suppression include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, Notch 4, ATK1 (v-alet murine thymoma viral oncogene homolog 1), ATK2 (v-alet murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R(fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia)), VHL (von-Hippel-Lindau tumor suppressor), BRCA1 (breast cancer 1), BRCA2 (breast cancer 2), AR (androgen receptor), TSG101 (tumor susceptibility gene 101), Igf1 (insulin-like growth factor 1), Igf2 (insulin-like growth factor 2), Igf 1R (insulin-like growth factor 1 receptor), Igf2R (insulin-like growth factor 2 receptor) Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7(Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), p53, TNF (tumor necrosis factor (TNF superfamily, member 2)), TP53 (tumor protein p53), ERBB2 (v-erb-b2 erythroblastic leukemia viral oncogene homolog 2, neuro/glioblastoma derived oncogene homolog (avian)), FN1 (fibronectin 1), TSC1 (tuberous sclerosis 1), PTGS2 (prostaglandin-endoperoxide synthase 2 (prostaglandin G/H synthase and cyclooxygenase)), PTEN (phosphatase and tensin homolog), PCNA (proliferating cell nuclear antigen), COL18A1 (collagen, type XVIII, alpha 1), TSSC4 (tumor suppressing subtransferable candidate 4), JUN (jun oncogene), MAPK8 (mitogen-activated protein kinase 8), TGFB1 (transforming growth factor, beta 1), IL6 (interleukin 6 (interferon, beta 2)), IFNG (interferon, gamma), BRCA1 (breast cancer 1, early onset), TSPAN32 (tetraspanin 32), BCL2 (B-cell CLL/lymphoma 2), NF2 (neurofibromin 2 (merlin)), GJB1 (gap junction protein, beta 1, 32 kDa), MAPK1 (mitogen-activated protein kinase 1), CD44 (CD44 molecule (Indian blood group)), PGR (progesterone receptor), TNS1 (tensin 1), PROK1 (prokineticin 1), SIAH1 (seven in absentia homolog 1 (Drosophila)), ENG (endoglin), TP73 (tumor protein p73), APC (adenomatous polyposis coli), BAX (BCL2-associated X protein), SRC (v-src sarcoma (Schmidt-Ruppin A-2) viral oncogene homolog (avian)), VHL (von Rippel-Lindau tumor suppressor), FHIT (fragile histidine triad gene), NFKB1 (nuclear factor of kappa light polypeptide gene enhancer in B-cells 1), IFNα1 (interferon, alpha 1), TGFBR1 (transforming growth factor, beta receptor 1), PRKCD (protein kinase C, delta), TGIF1 (TGFB-induced factor homeobox 1), DLC1 (deleted in liver cancer 1), SLC22A18 (solute carrier family 22, member 18), VEGFA (vascular endothelial growth factor A), MME (membrane metallo-endopeptidase), IL3 (interleukin 3 (colony-stimulating factor, multiple)), MK167 (antigen identified by monoclonal antibody Ki-67), HSPD1 (heat shock 60 kDa protein 1 (chaperonin)), HSPB1 (heat shock 27 kDa protein 1), HSP90B2P (heat shock protein 90 kDa beta (Grp94), member 2 (pseudogene)), MBL2 (mannose-binding lectin (protein C) 2, soluble (opsonic defect)), ZFYVE9 (zinc finger, FYVE domain containing 9), TERT (telomerase reverse transcriptase), PML (promyelocytic leukemia), SKP2 (S-phase kinase-associated protein 2 (p45)), CYCS (cytochrome c, somatic), MAPK10 (mitogen-activated protein kinase 10), PAX7 (paired box 7), YAP1 (Yes-associated protein 1), PARP1 (poly (ADP-ribose) polymerase 1), MIR34A (microRNA 34a), PRKCA (protein kinase C, alpha), FAS (Fas (TNF receptor superfamily, member 6)), SYK (spleen tyrosine kinase), GSK3B (glycogen synthase kinase 3 beta), PRKCE (protein kinase C, epsilon), CYP19A1 (cytochrome P450, family 19, subfamily A, polypeptide 1), ABCB1 (ATP-binding cassette, sub-family B (MDR/TAP), member 1), NFKBIA (nuclear factor of kappa light polypeptide gene enhancer in B-cells inhibitor, alpha), RUNX1 (runt-related transcription factor 1), PRKCG (protein kinase C, gamma), RELA (v-rel reticuloendotheliosis viral oncogene homolog A (avian)), PLAU (plasminogen activator, urokinase), BTK (Bruton agammaglobulinemia tyrosine kinase), PRKCB (protein kinase C, beta), CSF1 (colony stimulating factor 1 (macrophage)), POMC (proopiomelanocortin), CEBPB (CCAAT/enhancer binding protein (C/EBP), beta), ROCK1 (Rho-associated, coiled-coil containing protein kinase 1), KDR (kinase insert domain receptor (a type 111 receptor tyrosine kinase)), NPM1 (nucleophosmin (nucleolar phosphoprotein B23, numatrin)), ROCK2 (Rho-associated, coiled-coil containing protein kinase 2), PRKAB1 (protein kinase, AMP-activated, beta 1 non-catalytic subunit), BAK1 (BCL2-antagonist/killer 1), AURKA (aurora kinase A), NTN1 (netrin 1), FLT1 (fms-related tyrosine kinase 1 (vascular endothelial growth factor/vascular permeability factor receptor)), NBN (nibrin), DNM3 (dynamin 3), PRDM10 (PR domain containing 10), PAX5 (paired box 5), EIF4G1 (eukaryotic translation initiation factor 4 gamma, 1), KAT2B (K(lysine) acetyltransferase 2B), TIMP3 (TIMP metallopeptidase inhibitor 3), CCL22 (chemokine (C-C motif) ligand 22), GRIN2B (glutamate receptor, ionotropic, N-methyl D-aspartate 2B), CD81 (CD81 molecule), CCL27 (chemokine (C-C motif) ligand 27), MAPK11 (mitogen-activated protein kinase 11), DKK1 (dickkopf homolog 1 (Xenopus laevis)), HYAL1 (hyaluronoglucosaminidase 1), CTSL1 (cathepsin L), PKD1 (polycystic kidney disease 1 (autosomal dominant)), BUB1B (budding uninhibited by benzimidazoles 1 homolog beta (yeast)), MPP1 (membrane protein, palmitoylated 1, 55 kDa), SIAH2 (seven in absentia homolog 2 (Drosophila)), DUSP13 (dual specificity phosphatase 13), CCL21 (chemokine (C-C motif) ligand 21), RTN4 (reticulon 4), SMO (smoothened homolog (Drosophila)), CCL19 (chcmokinc (C-C motif) ligand 19), CSTF2 (cleavage stimulation factor, 3\′ pre-RNA, subunit 2, 64 kDa), RSF1 (remodeling and spacing factor 1), EZH2 (enhancer of zeste homolog 2 (Drosophila)), AK1 (adenylate kinase 1), CKM (creatine kinase, muscle), HYAL3 (hyaluronoglucosaminidase 3), ALOX15B (arachidonate 15-lipoxygenase, type B), PAG1 (phosphoprotein associated with glycosphingolipid microdomains 1), MIR21 (microRNA 21), S100A2 (S100 calcium binding protein A2), HYAL2 (hyaluronoglucosaminidase 2), CSTF1 (cleavage stimulation factor, 3V pre-RNA, subunit 1, 50 kDa), PCGF2 (polycomb group ring finger 2), THSD1 (thrombospondin, type I, domain containing 1), HOPX (HOP homeobox), SLC5A8 (solute carrier family 5 (iodide transporter), member 8), EMB (embigin homolog (mouse)), PAX9 (paired box 9), ARMCX3 (armadillo repeat containing, X-linked 3), ARMCX2 (armadillo repeat containing, X-linked 2), ARMCX1 (armadillo repeat containing, X-linked 1), RASSF4 (Ras association (RalGDS/AF-6) domain family member 4), MIR34B (microRNA 34b), MIR205 (microRNA 205), RB1 (retinoblastoma 1), DYT10 (dystonia 10), CDKN2A (cyclin-dependent kinase inhibitor 2A (melanoma, p16, inhibits CDK4)), CDKN1A (cyclin-dependent kinase inhibitor 1A (p21, Cip1)), CCND1 (cyclin D1), AKT1 (v-akt murine thymoma viral oncogene homolog 1), MYC (v-myc myelocytomatosis viral oncogene homolog (avian)), CTNNB1 (catenin (cadherin-associated protein), beta 1, 88 kDa), MDM2 (Mdm2 p53 binding protein homolog (mouse)), SERPINB5 (serpin peptidase inhibitor, clade B (ovalbumin), member 5), EGF (epidermal growth factor (beta-urogastrone)), FOS (FBJ murine osteosarcoma viral oncogene homolog), NOS2 (nitric oxide synthase 2, inducible), CDK4 (cyclin-dependent kinase 4), SOD2 (superoxide dismutase 2, mitochondrial), SMAD3 (SMAD family member 3), CDKN1B (cyclin-dependent kinase inhibitor 1B (p27, Kip1)), SOD1 (superoxide dismutase 1, soluble), CCNA2 (cyclin A2), LOX (lysyl oxidase), SMAD4 (SMAD family member 4), HGF (hepatocyte growth factor (hepapoietin A; scatter factor)), THBS 1 (thrombospondin 1), CDK6 (cyclin-dependent kinase 6), ATM (ataxia telangiectasia mutated), STAT3 (signal transducer and activator of transcription 3 (acute-phase response factor)), HIF1A (hypoxia inducible factor 1, alpha subunit (basic helix-loop-helix transcription factor)), IGF1R (insulin-like growth factor 1 receptor), MTOR (mechanistic target of rapamycin (serine/threonine kinase)), TSC2 (tuberous sclerosis 2), CDC42 (cell division cycle 42 (GTP binding protein, 25 kDa)), ODC1 (ornithine decarboxylase 1), SPARC (secreted protein, acidic, cysteine-rich (osteonectin)), HDAC1 (histone deacetylase 1), CDK2 (cyclin-dependent kinase 2), BARD1 (BRCA1 associated RING domain 1), CDH1 (cadherin 1, type 1, E-cadherin (epithelial)), EGR1 (early growth response 1), INSR (insulin receptor), IRF1 (interferon regulatory factor 1), PHB (prohibitin), PXN (paxillin), HSPA4 (heat shock 70 kDa protein 4), TYR (tyrosinase (oculocutaneous albinism IA)), CAV1 (caveolin 1, caveolae protein, 22 kDa), CDKN2B (cyclin-dependent kinase inhibitor 2B (p15, inhibits CDK4)), FOX03 (forkhead box O3), HDAC9 (histone deacetylase 9), FBXW7 (F-box and WD repeat domain containing 7), FOX01 (forkhead box O1), E2F1 (E2F transcription factor 1), STK11 (serine/threonine kinase 11), BMP2 (bone morphogenetic protein 2), HSP90AA1 (heat shock protein 90 kDa alpha (cytosolic), class A member 1), HNF4A (hepatocyte nuclear factor 4, alpha), CAMK2G (calcium/calmodulin-dependent protein kinase II gamma), TP53BP1 (tumor protein p53 binding protein 1), CRYAB (crystallin, alpha B), HMGCR (3-hydroxy-3-mcthylglutaryl-Coenzyme A reductase), PLAUR (plasminogen activator, urokinase receptor), MCL1 (myeloid cell leukemia sequence 1 (BCL2-related)), NOTCH1 (Notch homolog 1, translocation-associated (Drosophila)), RASSF1 (Ras association (RalGDS/AF-6) domain family member 1), GSN (gelsolin), CADM1 (cell adhesion molecule 1), ATF2 (activating transcription factor 2), IFNB1 (interferon, beta 1, fibroblast), DAPK1 (death-associated protein kinase 1), CHFR (checkpoint with forkhead and ring finger domains), KITLG (KIT ligand), NDUFA13 (NADH dehydrogenase (ubiquinone) 1 alpha subcomplex, 13), DPP4 (dipeptidyl-peptidase 4), GLB1 (galactosidase, beta 1), IKZF1 (IKAROS family zinc finger 1 (Ikaros)), ST5 (suppression of tumorigenicity 5), TGFA (transforming growth factor, alpha), EIF4EBP1 (eukaryotic translation initiation factor 4E binding protein 1), TGFBR2 (transforming growth factor, beta receptor II (70/80 kDa)), EIF2AK2 (eukaryotic translation initiation factor 2-alpha kinase 2), GJA1 (gap junction protein, alpha 1, 43 kDa), MYD88 (myeloid differentiation primary response gene (88)), IF127 (interferon, alpha-inducible protein 27), RBMX (RNA binding motif protein, X-linked), EPHA1 (EPH receptor A1), TWSG1 (twisted gastrulation homolog 1 (Drosophila)), H2AFX (H2A histone family, member X), LGALS3 (lectin, galactoside-binding, soluble, 3), MUC3A (mucin 3A, cell surface associated), ILK (integrin-linked kinase), APAF1 (apoptotic peptidase activating factor 1), MAOA (monoamine oxidase A), ERBB3 (v-erb-b2 erythroblastic leukemia viral oncogene homolog 3 (avian)), EIF2S 1 (eukaryotic translation initiation factor 2, subunit 1 alpha, 35 kDa), PER2 (period homolog 2 (Drosophila)), IGFBP7 (insulin-like growth factor binding protein 7), KDM5B (lysine (K)-specific demethylase 5B), SMARCA4 (SWI/SNF related, matrix associated, actin dependent regulator of chromatin, subfamily a, member 4), NME1 (non-metastatic cells 1, protein (NM23A) expressed in), F2RL1 (coagulation factor II (thrombin) receptor-like 1), ZFP36 (zinc finger protein 36, C3H type, homolog (mouse)), HSPA8 (heat shock 70 kDa protein 8), WNT5A (wingless-type MMTV integration site family, member 5A), ITGB4 (integrin, beta 4), RARB (retinoic acid receptor, beta), VEGFC (vascular endothelial growth factor C), CCL20 (chemokine (C-C motif) ligand 20), EPHB2 (EPH receptor B2), CSNK2A1 (casein kinase 2, alpha 1 polypeptide), PSMD9 (proteasome (prosome, macropain) 26S subunit, non-ATPase, 9), SERPINB2 (serpin peptidase inhibitor, clade B (ovalbumin), member 2), RHOB (ras homolog gene family, member B), DUSP6 (dual specificity phosphatase 6), CDKN1C (cyclin-dependent kinase inhibitor IC (p57, Kip2)), SLIT2 (slit homolog 2 (Drosophila)), CEACAM1 (carcinoembryonic antigen-related cell adhesion molecule 1 (biliary glycoprotein)), UBC (ubiquitin C), STS (steroid sulfatase (microsomal), isozyme S), FST (follistatin), KRT1 (keratin 1), ETF6 (eukaryotic translation initiation factor 6), JUP (junction plakoglobin), HDAC4 (histone deacetylase 4), NEDD4 (neural precursor cell expressed, developmentally down-regulated 4), KRT14 (keratin 14), GLI2 (GLI family zinc finger 2), MYH11 (myosin, heavy chain 11, smooth muscle), MAPKAPK5 (mitogen-activated protein kinase-activated protein kinase 5), MAD1L1 (MAD1 mitotic arrest deficient-like 1 (yeast)), TNFAIP3 (tumor necrosis factor, alpha-induced protein 3), WEE1 (WEE1 homolog (S. pombe)), BTRC (beta-transducin repeat containing), NKX3-1 (NK3 homeobox 1), GPC3 (glypican 3), CREB3 (cAMP responsive element binding protein 3), PLCB3 (phospholipase C, beta 3 (phosphatidylinositol-specific)), DMPK (dystrophia myotonica-protein kinase), BLNK (B-celllinker), PPIA (peptidylprolyl isomerase A (cyclophilin A)), DAB2 (disabled homolog 2, mitogen-responsive phosphoprotein (Drosophila)), KLF4 (Kruppel-like factor 4 (gut)), RUNX3 (runt-related transcription factor 3), FLG (filaggrin), IVL (involucrin), CCT5 (chaperonin containing TCP1, subunit 5 (epsilon)), LRPAP1 (low density lipoprotein receptor-related protein associated protein 1), IGF2R (insulin-like growth factor 2 receptor), PERI (period homolog 1 (Drosophila)), BIK (BCL2-interacting killer (apoptosis-inducing)), PSMC4 (proteasome (prosome, macropain) 26S subunit, ATPase, 4), USF2 (upstream transcription factor 2, c-fos interacting), GAS1 (growth arrest-specific 1), LAMP2 (lysosomal-associated membrane protein 2), PSMD10 (proteasome (prosome, macropain) 26S subunit, non-ATPase, 10), IL24 (interleukin24), GADD45G (growth arrest and DNA-damage-inducible, gamma), ARHGAP1 (Rho GTPase activating protein 1), CLDN1 (claudin 1), ANXA7 (annexin A7), CHN1 (chimerin (chimaerin) 1), TXNIP (thioredoxin interacting protein), PEG3 (paternally expressed 3), EIF3A (eukaryotic translation initiation factor 3, subunit A), CASC5 (cancer susceptibility candidate 5), TCF4 (transcription factor 4), CSNK2A2 (casein kinase 2, alpha prime polypeptide), CSNK2B (casein kinase 2, beta polypeptide), CRY1 (cryptochrome 1 (photolyase-like)), CRY2 (cryptochrome 2 (photolyase-like)), EIF4G2 (eukaryotic translation initiation factor 4 gamma, 2), LOXL2 (lysyl oxidase-like 2), PSMD13 (proteasome (prosome, macropain) 26S subunit, non-ATPase, 13), ANP32A (acidic (leucine-rich) nuclear phosphoprotein 32 family, member A), COL4A3 (collagen, type IV, alpha 3 (Goodpasture antigen)), SCGB1A1 (secretoglobin, family 1A, member 1 (uteroglobin)), BNIP3L (BCL2/adenovirus E1B 19 kDa interacting protein 3-like), MCC (mutated in colorectal cancers), EFNB3 (ephrin-B3), RBBP8 (retinoblastoma binding protein 8), PALB2 (partner and localizer of BRCA2), HBP1 (HMG-box transcription factor 1), MRPL28 (mitochondrial ribosomal protein L28), KDM5A (lysine (K)-specific demethylase SA), QSOX1 (quiescin Q6 sulfhydryl oxidase 1), ZFR (zinc finger RNA binding protein), MN1 (meningioma (disrupted in balanced translocation) 1), SMYD4 (SET and MYND domain containing 4), USP7 (ubiquitin specific peptidase 7 (herpes virus-associated)), STK4 (serine/threonine kinase 4), THY1 (Thy-1 cell surface antigen), PTPRG (protein tyrosine phosphatase, receptor type, G), E2F6 (E2F transcription factor 6), STX11 (syntaxin 11), CDC42BPA (CDC42 binding protein kinase alpha (DMPK-like)), MYOCD (myocardin), DAP (death-associated protein), LOXL1 (lysyl oxidase-like 1), RNF139 (ring finger protein 139), HTATIP2 (HIV-1 Tat interactive protein 2, 30 kDa), AIM1 (absent in melanoma 1), BCC1P (BRCA2 and CDKN1A interacting protein), LOXL4 (lysyl oxidase-like 4), WWC1 (WW and C2 domain containing 1), LOXL3 (lysyl oxidase-like 3), CENPN (centromere protein N), TNS4 (tensin 4), SIK1 (salt-inducible kinase 1), PCGF6 (polycomb group ring finger 6), PHLDA3 (pleckstrin homology-like domain, family A, member 3), IL32 (interleukin 32), LATS1 (LATS, large tumor suppressor, homolog 1 (Drosophila)), COMMD7 (COMM domain containing 7), CDHR2 (cadherin-related family member 2), LELP1 (late cornified envelope-like proline-rich 1), NCRNA00188 (non-protein coding RNA 188), and ENSG00000131023, and combinations thereof.

Examples of proteins associated with a secretase disorder include PSENEN (presenilin enhancer 2 homolog (C. elegans)), CTSB (cathepsin B), PSEN1 (prcscnilin 1), APP (amyloid beta (A4) precursor protein), APH1B (anterior pharynx defective 1 homolog B (C. elegans)), PSEN2 (presenilin 2 (Alzheimer disease 4)), BACE1 (beta-site APP-cleaving enzyme 1), ITM2B (integral membrane protein 2B), CTSD (cathepsin D), NOTCH1 (Notch homolog 1, translocation-associated (Drosophila)), TNF (tumor necrosis factor (TNF superfamily, member 2)), INS (insulin), DYT10 (dystonia 10), ADAM17 (ADAM metallopeptidase domain 17), APOE (apolipoprotein E), ACE (angiotensin I converting enzyme (peptidyl-dipeptidase A) 1), STN (statin), TP53 (tumor protein p53), IL6 (interleukin 6 (interferon, beta 2)), NGFR (nerve growth factor receptor (TNFR superfamily, member 16)), IL1B (interleukin 1, beta), ACHE (acetylcholinesterase (Yt blood group)), CTNNB1 (catenin (cadherin-associated protein), beta 1, 88 kDa), IGF1 (insulin-like growth factor 1 (somatomedin C)), IFNG (interferon, gamma), NRG1 (neuregulin 1), CASP3 (caspase 3, apoptosis-related cysteine peptidase), MAPK1 (mitogen-activated protein kinase 1), CDH1 (cadherin 1, type 1, E-cadherin (epithelial)), APBB1 (amyloid beta (A4) precursor protein-binding, family B, member 1 (Fe65)), HMGCR (3-hydroxy-3-methylglutaryl-Coenzyme A reductase), CREB1 (cAMP responsive element binding protein 1), PTGS2 (prostaglandin-endoperoxide synthase 2 (prostaglandin G/H synthase and cyclooxygenase)), HES1 (hairy and enhancer of split 1, (Drosophila)), CAT (catalase), TGFB1 (transforming growth factor, beta 1), EN02 (enolase 2 (gamma, neuronal)), ERBB4 (v-erb-a erythroblastic leukemia viral oncogene homolog 4 (avian)), TRAPPC10 (trafficking protein particle complex 10), MAOB (monoamine oxidase B), NGF (nerve growth factor (beta polypeptide)), MMP12 (matrix metallopeptidase 12 (macrophage elastase)), JAG1 (jagged 1 (Alagille syndrome)), CD40LG (CD40 ligand), PPARG (peroxisome proliferator-activated receptor gamma), FGF2 (fibroblast growth factor 2 (basic)), IL3 (interleukin3 (colony-stimulating factor, multiple)), LRP1 (low density lipoprotein receptor-related protein 1), NOTCH4 (Notch homolog 4 (Drosophila)), MAPKS (mitogen-activated protein kinase 8), PREP (prolyl endopeptidase), NOTCH3 (Notch homolog 3 (Drosophila)), PRNP (prion protein), CTSG (cathepsin G), EGF (epidermal growth factor (beta-urogastrone)), REN (renin), CD44 (CD44 molecule (Indian blood group)), SELP (selectin P (granule membrane protein 140 kDa, antigen CD62)), GHR (growth hormone receptor), ADCYAP1 (adenylate cyclase activating polypeptide 1 (pituitary)), INSR (insulin receptor), GFAP (glial fibrillary acidic protein), MMP3 (matrix metallopeptidase 3 (stromelysin 1, progelatinase)), MAPK10 (mitogen-activated protein kinase 10), SP1 (Spl transcription factor), MYC (v-myc myelocytomatosis viral oncogene homolog (avian)), CTSE (cathepsin E), PPARA (peroxisome proliferator-activated receptor alpha), JUN (jun oncogene), TIMP1 (TIMP metallopeptidase inhibitor 1), IL5 (interleukin 5 (colony-stimulating factor, eosinophil)), ILIA (interleukin 1, alpha), MMP9 (matrix metallopeptidase 9 (gelatinase B, 92 kDa gelatinase, 92 kDa type IV collagenase)), HTR4 (5-hydroxytryptamine (serotonin) receptor 4), HSPG2 (heparan sulfate proteoglycan 2), KRAS (v-Ki-ras2 Kirsten rat sarcoma viral oncogene homolog), CYCS (cytochrome c, somatic), SMG1 (SMG1 homolog, phosphatidylinositol 3-kinase-related kinase (C. elegans)), IL1R1 (interleukin 1 receptor, type I), PROK1 (prokineticin 1), MAPK3 (mitogen-activated protein kinase 3), NTRK1 (neurotrophic tyrosine kinase, receptor, type 1), IL13 (interleukin 13), MME (membrane metallo-endopeptidase), TKT (transketolase), CXCR2 (chemokine (C-X-C motif) receptor 2), IGF1R (insulin-like growth factor 1 receptor), RARA (retinoic acid receptor, alpha), CREBBP (CREB binding protein), PTGS 1 (prostaglandin-endoperoxide synthase 1 (prostaglandin G/H synthase and cyclooxygenase)), GALT (galactose-1-phosphate uridylyltransferase), CHRM1 (cholinergic receptor, muscarinic 1), ATXN1 (ataxin 1), PAWR (PRKC, apoptosis, WT1, regulator), NOTCH2 (Notch homolog 2 (Drosophila)), M6PR (mannose-6-phosphate receptor (cation dependent)), CYP46A1 (cytochrome P450, family 46, subfamily A, polypeptide 1), CSNK1D (casein kinase 1, delta), MAPK14 (mitogen-activated protein kinase 14), PRG2 (proteoglycan 2, bone marrow (natural killer cell activator, eosinophil granule major basic protein)), PRKCA (protein kinase C, alpha), L1CAM (L1 cell adhesion molecule), CD40 (CD40 molecule, TNF receptor superfamily member 5), NR112 (nuclear receptor subfamily 1, group I, member 2), JAG2 (jagged 2), CTNND1 (catenin (cadherin-associated protein), delta 1), CDH2 (cadherin 2, type 1, N-cadherin (neuronal)), CMA1 (chymase 1, mast cell), SORT1 (sortilin 1), DLK1 (delta-like 1 homolog (Drosophila)), THEM4 (thioesterase superfamily member 4), JUP (junction plakoglobin), CD46 (CD46 molecule, complement regulatmy protein), CCL11 (chemokine (C-C motif) ligand 11), CAV3 (caveolin 3), RNASE3 (ribonuclease, RNase A family, 3 (eosinophil cationic protein)), HSPAS (heat shock 70 kDa protein 8), CASP9 (caspase 9, apoptosis-related cysteine peptidase), CYP3A4 (cytochrome P450, family 3, subfamily A, polypeptide 4), CCR3 (chemokine (C-C motif) receptor 3), TFAP2A (transcription factor AP-2 alpha (activating enhancer binding protein 2 alpha)), SCP2 (sterol carrier protein 2), CDK4 (cyclin-dependent kinase 4), HIF1A (hypoxia inducible factor 1, alpha subunit (basic helix-loop-helix transcription factor)), TCF7L2 (transcription factor 7-like 2 (T-cell specific, HMG-box)), IL1R2 (interleukin 1 receptor, type II), B3GALTL (beta 1,3-galactosyltransferase-like), MDM2 (Mdm2 p53 binding protein homolog (mouse)), RELA (v-rel reticuloendotheliosis viral oncogene homolog A (avian)), CASP7 (caspase 7, apoptosis-related cysteine peptidase), IDE (insulin-degrading enzyme), FABP4 (fatty acid binding protein 4, adipocyte), CASK (calcium/calmodulin-dependent serine protein kinase (MAGUK family)), ADCYAP1R1 (adenylate cyclase activating polypeptide 1 (pituitary) receptor type I), ATF4 (activating transcription factor 4 (tax-responsive enhancer element B67)), PDGFA (platelet-derived growth factor alpha polypeptide), C21orf33 (chromosome 21 open reading frame 33), SCG5 (secretogranin V (7B2 protein)), RNF123 (ring finger protein 123), NFKB1 (nuclear factor of kappa light polypeptide gene enhancer in B-cells 1), ERBB2 (v-erb-b2 erythroblastic leukemia viral oncogene homolog 2, neuro/glioblastoma derived oncogene homolog (avian)), CAV1 (caveolin 1, caveolae protein, 22 kDa), MMP7 (matrix metallopeptidase 7 (matrilysin, uterine)), TGFα (transforming growth factor, alpha), RXRA (retinoid X receptor, alpha), STX1A (syntaxin 1A (brain)), PSMC4 (proteasome (prosome, macropain) 26S subunit, ATPase, 4), P2RY2 (purinergic receptor P2Y, G-protein coupled, 2), TNFRSF21 (tumor necrosis factor receptor superfamily, member 21), DLG1 (discs, large homolog 1 (Drosophila)), NUMBL (numb homolog (Drosophila)-like), SPN (sialophorin), PLSCR1 (phospholipid scramblase 1), UBQLN2 (ubiquilin 2), UBQLN1 (ubiquilin 1), PCSK7 (proprotein convertase subtilisin/kexin type 7), SPON1 (spondin 1, extracellular matrix protein), SILV (silver homolog (mouse)), QPCT (glutaminyl-peptide cyclotransferase), HESS (hairy and enhancer of split 5 (Drosophila)), GCC1 (GRIP and coiled-coil domain containing 1), and any combination thereof.

Examples of proteins associated with Amyotrophic Lateral Sclerosis include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins associated with prion diseases include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof. Examples of proteins related to neurodegenerative conditions in prion disorders include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), ADRA1D (Alpha-1D adrenergic receptor for Alpha-1D adrenoreceptor), AHSG (Alpha-2-HS-glycoprotein), A1F1 (Allograft inflammatory factor 1), ALAS2 (Delta-aminolevulinate synthase 2), AMBP (Alpha-1-microglobulin/bikunin precursor), ANK3 (Ankryn 3), ANXA3 (Annexin A3), APCS (Amyloid P component serum), APOA1 (Apolipoprotein A1), APOA12 (Apolipoprotein A2), APOB (Apolipoprotein B), APOC1 (Apolipoprotein C1), APOE (Apolipoprotein E), APOH (Apolipoprotein H), APP (Amyloid precursor protein), ARC (Activity-regulated cytoskeleton-associated protein), ARF6 (ADP-ribosylation factor 6), ARHGAP5 (Rho GTPase activating protein 5), ASCL1 (Achaete-scute homolog 1), B2M (Beta-2 microglobulin), B4GALNT1 (Beta-1,4-N-acetyl-galactosaminyl transferase 1), BAX (Bel-2-associated X protein), BCAT (Branched chain amino-acid transaminase 1 cytosolic), BCKDHA (Branched chain keto acid dehydrogenase E1 alpha), BCKDK (Branched chain alpha-ketoacid dehydrogenase kinase), BCL2 (B-celllymphoma 2), BCL2L1 (BCL2-like 1), BDNF (Brain-derived neurotrophic factor), BHLHE40 (Class E basic helix-loop-helix protein 40), BHLHE41 (Class E basic helix-loop-helix protein 41), BMP2 (Bone morphogenetic protein 2A), BMP3 (Bone morphogenetic protein 3), BMP5 (Bone morphogenetic protein 5), BRD1 (Bromodomain containing 1), BTC (Betacellulin), BTNL8 (Butyrophilin-like protein 8), CALB1 (Calbindin 1), CALM1 (Calmodulin 1), CAMK1 (Calcium/calmodulin-dependent protein kinase type I), CAMK4 (Calcium/calmodulin-dependent protein kinase type IV), CAMKIIB (Calcium/calmodulin-dependent protein kinase type IIB), CAMKIIG (Calcium/calmodulin-dependent protein kinase type IIG), CASP11 (Caspase-10), CASP8 (Caspase 8 apoptosis-related cysteine peptidase), CBLN1 (cerebellin 1 precursor), CCL2 (Chemokine (C-C motif) ligand 2), CCL22 (Chemokine (C-C motif) ligand 22), CCL3 (Chemokine (C-C motif) ligand 3), CCL8 (Chemokine (C-C motif) ligand 8), CCNG1 (Cyclin-GI), CCNT2 (Cyclin T2), CCR4 (C-C chemokine receptor type 4 (CD194)), CD58 (CD58), CD59 (Protectin), CD5L (CD5 antigen-like), CD93 (CD93), CDKN2AIP (CDKN2A interacting protein), CDKN2B (Cyclin-dependent kinase inhibitor 2B), CDX1 (Homeobox protein CDX-1), CEA (Carcinoembryonic antigen), CEBPA (CCAAT/enhancer-binding protein alpha), CEBPB (CCAAT/enhancer binding protein C/EBP beta), CEBPB (CCAAT/enhancer-binding protein beta), CEBPD (CCAAT/enhancer-binding protein delta), CEBPG (CCAAT/enhancer-binding protein gamma), CENPB (Centromere protein B), CGA (Glycoprotein hormone alpha chain), CGGBP1 (CGG triplet repeat-binding protein 1), CHGA (Chromogranin A), CHGB (Secretoneurin), CHN2 (Beta-chimaerin), CHRD (Chordin), CHRM1 (Cholinergic receptor muscarinic 1), CITED2 (Cbp/p300-interacting transactivator 2), CLEC4E (C-type lectin domain family 4 member E), CMTM2 (CKLF-like MARVEL transmembrane domain-containing protein 2), CNTN1 (Contactin 1), CNTNAP1 (Contactin-associated protein-like 1), CR1 (Erythrocyte complement receptor 1), CREM (cAMP-responsive element modulator), CRH (Corticotropin-releasing hormone), CRHR1 (Corticotropin releasing hormone receptor 1), CRKRS (Cell division cycle 2-related protein kinase 7), CSDA (DNA-binding protein A), CSF3 (Granulocyte colony stimulating factor 3), CSF3R (Granulocyte colony-stimulating factor 3 receptor), CSP (Chemosensory protein), CSPG4 (Chondroitin sulfate proteoglycan 4), CTCF (CCCTC-binding factor zinc finger protein), CTGF (Connective tissue growth factor), CXCL12 (Chemokine C-X-C motifligand 12), DAD1 (Defender against cell death 1), DAXX (Death associated protein 6), DBN1 (Drebrin 1), DBP (D site of albumin promoter-albumin D-box binding protein), DDR1 (Discoidin domain receptor family member 1), DDX14 (DEAD (SEQ ID NO: 532)/DEAN (SEQ ID NO: 533) box helicase), DEFA3 (Defensin alpha 3 neutrophil-specific), DVL3 (Dishevelled dsh homolog 3), EDN1 (Endothelin 1), EDNRA (Endothelin receptor type A), EGF (Epidermal growth factor), EGFR (Epidermal growth factor receptor), EGR1 (Early growth response protein 1), EGR2 (Early growth response protein 2), EGR3 (Early growth response protein 3), EIF2AK2 (Eukaryotic translation initiation factor 2-alpha kinase 2), ELANE (Elastase neutrophil expressed), ELK1 (ELK1 member of ETS oncogene family), ELK3 (ELK3 ETS-domain protein (SRF accessory protein 2)), EML2 (Echinoderm microtubule associated protein like 2), EPHA4 (EPH receptor A4), ERBB2 (V-erb-b2 erythroblastic leukemia viral oncogene homolog 2), ERBB3 (Receptor tyrosine-protein kinase erbB-3), ESR2 (Estrogen receptor 2), ESR2 (Estrogen receptor 2), ETS 1 (V-ets erythroblastosis virus E26 oncogene homolog 1), ETV6 (Ets variant 6), FASLG (Fas ligand TNF superfamily member 6), FCAR (Fe fragment of IgA receptor), FCER1G (Fc fragment of IgE high affinity 1 receptor for gamma polypeptide), FCGR2A (Fc fragment of IgG low affinity IIa receptor-CD32), FCGR3B (Fc fragment of IgG low affinity IIIb receptor-CD16b), FCGRT (Fc fragment of IgG receptor transporter alpha), FGA (Basic fibrinogen), FGF1 (Acidic fibroblast growth factor 1), FGF14 (Fibroblast growth factor 14), FGF16 (fibroblast growth factor 16), FGF18 (Fibroblast growth factor 18), FGF2 (Basic fibroblast growth factor 2), FIBP (Acidic fibroblast growth factor intracellular binding protein), FIGF (C-fos induced growth factor), FMR1 (Fragile X mental retardation 1), FOSB (FBJ murine osteosarcoma viral oncogene homolog B), FOXO1 (Forkhead box O1), FSHB (Follicle stimulating hormone beta polypeptide), FTH1 (Ferritin heavy polypeptide 1), FTL (Ferritin light polypeptide), G1P3 (Interferon alpha-inducible protein 6), G6S(N-acetylglucosamine-6-sulfatase), GABRA2 (Gamma-aminobutyric acid A receptor alpha 2), GABRA3 (Gamma-aminobutyric acid A receptor alpha 3), GABRA4 (Gamma-aminobutyric acid A receptor alpha 4), GABRB1 (Gamma-aminobutyric acid A receptor beta 1), GABRG1 (Gamma-aminobutyric acid A receptor gamma 1), GADD45A (Growth arrest and DNA-damage-inducible alpha), GCLC (Glutamate-cysteine ligase catalytic subunit), GDF15 (Growth differentiation factor 15), GDF9 (Growth differentiation factor 9), GFRA1 (GDNF family receptor alpha 1), GIT 1 (G protein-coupled receptor kinase interactor 1), GNA13 (Guanine nucleotide-binding protein/G protein alpha 13), GNAQ (Guanine nucleotide binding protein/G protein q polypeptide), GPR12 (G protein-coupled receptor 12), GPR18 (G protein-coupled receptor 18), GPR22 (G protein-coupled receptor 22), GPR26 (G protein-coupled receptor 26), GPR27 (G protein-coupled receptor 27), GPR77 (G protein-coupled receptor 77), GPR85 (G protein-coupled receptor 85), GRB2 (Growth factor receptor-bound protein 2), GRLF1 (Glucocorticoid receptor DNA binding factor 1), GST (Glutathione S-transferase), GTF2B (General transcription factor IIB), GZMB (Granzyme B), HAND1 (Heart and neural crest derivatives expressed 1), HAVCR1 (Hepatitis A virus cellular receptor 1), HES1 (Hairy and enhancer of split 1), HESS (Hairy and enhancer of split 5), HLA-DQA1 (Major histocompatibility complex class II DQ alpha), HOXA2 (Homeobox A2), HOXA4 (Homeobox A4), HP (Haptoglobin), HPGDS (Prostaglandin-D synthase), HSPA8 (Heat shock 70 kDa protein 8), HTR1A (5-hydroxytryptamine receptor 1A), HTR2A (5-hydroxytryptamine receptor 2A), HTR3A (5-hydroxytryptamine receptor 3A), ICAM1 (Intercellular adhesion molecule 1 (CD54)), IFIT2 (Interferon-induced protein with tetratricopeptide repeats 2), IFNAR2 (Interferon alpha/beta/omega receptor 2), IGF1 (Insulin-like growth factor 1), IGF2 (Insulin-like growth factor 2), IGFBP2 (Insulin-like growth factor binding protein 2, 36 kDa), IGFBP7 (Insulin-like growth factor binding protein 7), IL10 (Interleukin 10), IL1ORA (Interleukin 10 receptor alpha), IL11 (Interleukin 11), IL11RA (Interleukin 11 receptor alpha), IL11RB (Interleukin 11 receptor beta), IL13 (Interleukin 13), IL15 (Interleukin 15), IL17A (Interleukin 17A), IL17RB (interleukin 17 receptor B), IL18 (Interleukin 18), IL18RAP (Interleukin 18 receptor accessory protein), IL1R2 (Interleukin 1 receptor type II), ILIRN (Interleukin 1 receptor antagonist), IL2RA (Interleukin 2 receptor alpha), IL4R (Interleukin 4 receptor), IL6 (Interleukin 6), IL6R (Interleukin 6 receptor), IL7 (Interleukin 7), IL8 (Interleukin 8), IL8RA (Interleukin 8 receptor alpha), IL8RB (Interleukin 8 receptor beta), ILK (Integrin-linked kinase), INPP4A (Inositol polyphosphate-4-phosphatase type I, 107 kDa), INPP4B (Inositol polyphosphate-4-phosphatase type 1 beta), INS (Insulin), IRF2 (Interferon regulatory factor 2), IRF3 (Interferon regulatory factor 3), IRF9 (Interferon regulatory factor 9), IRS1 (Insulin receptor substrate 1), ITGA4 (integrin alpha 4), ITGA6 (Integrin alpha-6), ITGAE (Integrin alpha E), ITGAV (Integrin alpha-V), JAG1 (Jagged 1), JAK1 (Janus kinase 1), JDP2 (Jun dimerization protein 2), JUN (Jun oncogene), JUNB (Jun B proto-oncogene), KCNJ15 (Potassium inwardly-rectifying channel subfamily J member 15), KTF5B (Kinesin family member 5B), KLRC4 (Killer cell lectin-like receptor subfamily C member 4), KRT8 (Keratin 8), LAMP2 (Lysosomal-associated membrane protein 2), LEP (Leptin), LHB (Luteinizing hormone beta polypeptide), LRRN3 (Leucine rich repeat neuronal 3), MAL (Mal T-cell differentiation protein), MAN1A1 (Mannosidase alpha class 1A member 1), MAOB (Monoamine oxidase B), MAP3K1 (Mitogen-activated protein kinase kinase kinase 1), MAPK1 (Mitogen-activated protein kinase 1), MAPK3 (Mitogen-activated protein kinase 3), MAPRE2 (Microtubule-associated protein RP/EB family member 2), MARCKS (Myristoylated alanine-rich protein kinase C substrate), MAS 1 (MAS 1 oncogene), MASL1 (MAS 1 oncogene-like), MBP (Myelin basic protein), MCL1 (Myeloid cell leukemia sequence 1), MDMX (MDM2-like p53-binding protein), MECP2 (Methyl CpG binding protein 2), MFGE8 (Milk fat globule-EGF factor 8 protein), MIF (Macrophage migration inhibitory factor), MMP2 (Matrix metallopeptidase 2), MOBP (Myelin-associated oligodendrocyte basic protein), MUC16 (Cancer antigen 125), MX2 (Myxovirus (influenza virus) resistance 2), MYBBP1A (MYB binding protein 1a), NBN (Nibrin), NCAM1 (Neural cell adhesion molecule 1), NCF4 (Neutrophil cytosolic factor 4 40 kDa), NCOA1 (Nuclear receptor coactivator 1), NCOA2 (Nuclear receptor coactivator 2), NEDD9 (Neural precursor cell expressed developmentally down-regulated 9), NEUR (Neuraminidase), NFATC1 (Nuclear factor of activated T-cells cytoplasmic calcineurin-dependent 1), NFE2L2 (Nuclear factor erythroid-derived 2-like 2), NFIC (Nuclear factor I/C), NFKBIA (Nuclear factor of kappa light polypeptide gene enhancer in B-cells inhibitor alpha), NGFR (Nerve growth factor receptor), NIACR2 (niacin receptor 2), NLGN3 (Neuroligin 3), NPFFR2 (neuropeptide FF receptor 2), NPY (Neuropeptide Y), NR3C2 (Nuclear receptor subfamily 3 group C member 2), NRAS (Neuroblastoma RAS viral (v-ras) oncogene homolog), NRCAM (Neuronal cell adhesion molecule), NRG1 (Neuregulin 1), NRTN (Neurturin), NRXN1 (Neurexin 1), NSMAF (Neutral sphingomyelinase activation associated factor), NTF3 (Neurotrophin 3), NTF5 (Neurotrophin 4/5), ODC1 (Ornithine decarboxylase 1), OR10A1 (Olfactory receptor 10A1), OR1A1 (Olfactory receptor family 1 subfamily A member 1), OR1N1 (Olfactory receptor family 1 subfamily N member 1), OR3A2 (Olfactory receptor family 3 subfamily A member 2), OR7A17 (Olfactory receptor family 7 subfamily A member 17), ORM1 (Orosomucoid 1), OXTR (Oxytocin receptor), P2RY13 (Purinergic receptor P2Y G-protein coupled 13), P2Y12 (Purinergic receptor P2Y G-protein coupled 12), P70S6K (P70S6 kinase), PAK1 (P21/Cdc42/Racl-activatedkinase 1), PAR1 (Prader-Willi/Angelman region-1), PBEF1 (Pre-B-cell colony enhancing factor 1), PCAF (P300/CBP-associated factor), PDE4A (cAMP-specific 3′,5′-cyclic phosphodiesterase 4A), PDE4B (Phosphodiesterase 4B cAMP-specific), PDE4B (Phosphodiesterase 4B cAMP-specific), PDE4D (Phosphodiesterase 4D cAMP-specific), PDGFA (Platelet-derived growth factor alpha polypeptide), PDGFB (Platelet-derived growth factor beta polypeptide), PDGFC (Platelet derived growth factor C), PDGFRB (Beta-type platelet-derived growth factor receptor), PDPN (Podoplanin), PENK (Enkephalin), PERI (Period homolog 1), PLA2 (Phospholipase A2), PLAU (Plasminogen activator urokinase), PLXNC1 (Plexin C1), PMVK (Phosphomevalonate kinase), PNOC (Prepronociceptin), POLH (Polymerase (DNA directed) eta), POMC (Proopiomelanocmiin (adrenocorticotropin/beta-lipotropin/alpha-melanocyte stimulating hormone/beta-melanocyte stimulating hormone/beta-endorphin)), POU2AF1 (POU domain class 2 associating factor 1), PRKAA1 (5′-AMP-activated protein kinase catalytic subunit alpha-1), PRL (Prolactin), PSCDBP (Cytohesin 1 interacting protein), PSPN (Persephin), PTAFR (Platelet-activating factor receptor), PTGS2 (Prostaglandin-endoperoxide synthase 2), PTN (Pleiotrophin), PTPN11 (Protein tyrosine phosphatase non-receptor type 11), PYY (Peptide YY), RAB11B (RAB11B member RAS oncogene family), RAB6A (RAB6A member RAS oncogene family), RAD17 (RAD17 homolog), RAF1 (RAF proto-oncogene serine/threonine-protein kinase), RANBP2 (RAN binding protein 2), RAP1A (RAP1A member of RAS oncogene family), RB1 (Retinoblastoma 1), RBL2 (Retinoblastoma-like 2 (p130)), RCVRN (Recoverin), REM2 (RAS/RAD/GEM-like GTP binding 2), RFRP (RFamide-related peptide), RPS6KA3 (Ribosomal protein S6 kinase 90 kDa polypeptide 3), RTN4 (Reticulon 4), RUNX1 (Runt-related transcription factor 1), S100A4 (S100 calcium binding protein A4), S1PR1 (Sphingosine-1-phosphate receptor 1), SCG2 (Secretogranin II), SCYE1 (Small inducible cytokine subfamily E member 1), SELENBP1 (Selenium binding protein 1), SGK (Serum/glucocorticoid regulated kinase), SKD1 (Suppressor of K+ transport growth defect 1), SLC14A1 (Solute carrier family 14 (urea transporter) member 1 (Kidd blood group)), SLC25A37 (Solute carrier family 25 member 37), SMAD2 (SMAD family member 2), SMAD5 (SMAD family member 5), SNAP23 (Synaptosomal-associated protein 23 kDa), SNCB (Synuclein beta), SNF1LK (SNF1-like kinase), SORT1 (Sortilin 1), SSB (Sjogren syndrome antigen B), STAT1 (Signal transducer and activator of transcription 1, 91 kDa), STAT5A (Signal transducer and activator of transcription 5A), STAT5B (Signal transducer and activator of transcription 5B), STX16 (Syntaxin 16), TAC1 (Tachykinin precursor 1), TBX1 (T-box 1), TEF (Thyrotrophic embryonic factor), TF (Transferrin), TGFA (Transforming growth factor alpha), TGFB1 (Transforming growth factor beta 1), TGFB2 (Transforming growth factor beta 2), TGFB3 (Transforming growth factor beta 3), TGFBR1 (Transforming growth factor beta receptor I), TGM2 (Transglutaminase 2), THPO (Thrombopoietin), TIMP1 (TIMP metallopeptidase inhibitor 1), TIMP3 (TIMP metallopeptidase inhibitor 3), TMEM129 (Transmembrane protein 129), TNFRC6 (TNFR/NGFR cysteine-rich region), TNFRSF10A (Tumor necrosis factor receptor superfamily member 10a), TNFRSF10C (Tumor necrosis factor receptor superfamily member 10c decoy without an intracellular domain), TNFRSF1A (Tumor necrosis factor receptor superfamily member 1A), TOB2 (Transducer of ERBB2 2), TOP1 (Topoisomerase (DNA) I), TOPOII (Topoisomerase 2), TRAK2 (Trafficking protein kinesin binding 2), TRH (Thyrotropin-releasing hormone), TSH (Thyroid-stimulating hormone alpha), TUBA1A (Tubulin alpha 1a), TXK (TXK tyrosine kinase), TYK2 (Tyrosine kinase 2), UCP1 (Uncoupling protein 1), UCP2 (Uncoupling protein 2), UL1P (Unc-33-like phosphoprotein), UTRN (Utrophin), VEGF (Vascular endothelial growth factor), VGF (VGF nerve growth factor inducible), VIP (Vasoactive intestinal peptide), VNN1 (Vanin 1), VTN (Vitronectin), WNT2 (Wingless-type MMTV integration site family member 2), XRCC6 (X-ray repair cross-complementing 6), ZEB2 (Zinc finger E-box binding homeobox 2), and ZNF461 (Zinc finger protein 461).

Examples of proteins associated with Immunodeficiency include A2M [alpha-2-macroglobulin]; AANAT [arylalkylarnine N-acetyltransferase]; ABCA 1 [ATP-binding cassette, sub-family A (ABC1), member 1]; ABCA2 [ATP-binding cassette, sub-family A (ABC1), member 2]; ABCA3 [ATP-binding cassette, sub-family A (ABC1), member 3]; ABCA4 [ATP-binding cassette, sub-family A (ABC1), member 4]; ABCB1 [ATP-binding cassette, sub-family B (MDR/TAP), member 1]; ABCC1 [ATP-binding cassette, sub-family C (CFTR/MRP), member 1]; ABCC2 [ATP-binding cassette, sub-family C (CFTR/MRP), member 2]; ABCC3 [ATP-binding cassette, sub-family C (CFTR/MRP), member 3]; ABCC4 [ATP-binding cassette, sub-family C (CFTR/MRP), member 4]; ABCC8 [ATP-binding cassette, sub-family C (CFTR/MRP), member 8]; ABCD2 [ATP-binding cassette, sub-family D (ALD), member 2]; ABCD3 [ATP-binding cassette, sub-family D (ALD), member 3]; ABCG1 [ATP-binding cassette, sub-family G (WHITE), member 1]; ABCC2 [ATP-binding cassette, sub-family G (WHITE), member 2]; ABCG5 [ATP-binding cassette, sub-family G (WHITE), member 5]; ABCC8 [ATP-binding cassette, sub-family G (WHITE), member 8]; ABHD2 [abhydrolase domain containing 2]; ABL1 [c-abl oncogene 1, receptor tyrosine kinase]; ABO [ABO blood group (transferase A, alpha 1-3-N-acetylgalactosaminyltransferase; transferase B, alpha 1-3-galactosyltransferase)]; ABP1 [amiloride binding protein 1 (amine oxidase (copper-containing))]; ACAA1 [acetyl-Coenzyme A acyltransferase 1]; ACACA [acetyl-Coenzyme A carboxylase alpha]; ACAN [aggrecan]; ACAT1 [acetyl-Coenzyme A acetyltransferase 1]; ACAT2 [acetyl-Coenzyme A acetyltransferase 2]; ACCN5 [amiloride-sensitive cation channel 5, intestinal]; ACE [angiotensin I converting enzyme (peptidyl-dipeptidase A) 1]; ACE2 [angiotensin I converting enzyme (peptidyl-dipeptidase A) 2]; ACHE [acetylcholinesterase (Yt blood group)]; ACLY [ATP citrate lyase]; ACOT9 [acyl-CoA thioesterase 9]; ACOX1 [acyl-Coenzyme A oxidase 1, palmitoyl]; ACP1 [acid phosphatase 1, soluble]; ACP2 [acid phosphatase 2, lysosomal]; ACP5 [acid phosphatase 5, tartrate resistant]; ACPP [acid phosphatase, prostate]; ACSL3 [acyl-CoA synthetase long-chain family member 3]; ACSM3 [acyl-CoA synthetase medium-chain family member 3]; ACTA1 [actin, alpha 1, skeletal muscle]; ACTA2 [actin, alpha 2, smooth muscle, aorta]; ACTB [actin, beta]; ACTC1 [actin, alpha, cardiac muscle 1]; ACTG1 [actin, gamma 1]; ACTN1 [actinin, alpha 1]; ACTN2 [actinin, alpha 2]; ACTN4 [actinin, alpha 4]; ACTR2 [ARP2 actin-related protein 2 homolog (yeast)]; ACVR1 [activin A receptor, type I]; ACVR1B [activin A receptor, type IB]; ACVRL1 [activin A receptor type II-like 1]; ACY1 [aminoacylase 1]; ADA [adenosine deaminase]; ADAM10 [ADAM metallopeptidase domain 10]; ADAM12 [ADAM metallopeptidase domain 12]; ADAM17 [ADAM metallopeptidase domain 17]; ADAM23 [ADAM metallopeptidase domain 23]; ADAM33 [ADAM metallopeptidase domain 33]; ADAM8 [ADAM metallopeptidase domain 8]; ADAM9 [ADAM metallopeptidase domain 9 (meltrin gamma)]; ADAMTS1 [ADAM metallopeptidase with thrombospondin type 1 motif, 1]; ADAMTS12 [ADAM metallopeptidase with thrombospondin type 1 motif, 12]; ADAMTS13 [ADAM metallopeptidase with thrombospondin type 1 motif, 13]; ADAMTS15 [ADAM metallopeptidase with thrombospondin type 1 motif, 15]; ADAMTSL1 [ADAMTS-like 1]; ADAMTSL4 [ADAMTS-like 4]; ADAR [adenosine deaminase, RNA-specific]; ADCY1 [adenylate cyclase 1 (brain)]; ADCY10 [adenylate cyclase 10 (soluble)]; ADCY3 [adenylate cyclase 3]; ADCY9 [adenylate cyclase 9]; ADCYAP1 [adenylate cyclase activating polypeptide 1 (pituitary)]; ADCYAP1 R1 [adenylate cyclase activating polypeptide 1 (pituitary) receptor type I]; ADD1 [adducin 1 (alpha)]; ADH5 [alcohol dehydrogenase 5 (class III), chi polypeptide]; ADIPOQ [adiponectin, C1Q and collagen domain containing]; ADIPOR1 [adiponectin receptor 1]; ADK [adenosine kinase]; ADM [adrenomedullin]; ADORA1 [adenosine A1 receptor]; ADORA2A [adenosine A2a receptor]; ADORA2B [adenosine A2b receptor]; ADORA3 [adenosine A3 receptor]; ADRA1B [adrenergic, alpha-1B-, receptor]; ADRA2A [adrenergic, alpha-2A-, receptor]; ADRA2B [adrenergic, alpha-2B-, receptor]; ADRB1 [adrenergic, beta-1-, receptor]; ADRB2 [adrenergic, beta-2-, receptor, surface]; ADSL [adenylosuccinate lyase]; ADSS [adenylosuccinate synthase]; AEBP1 [AE binding protein 1]; AFP [alpha-fetoprotein]; AGER [advanced glycosylation end product-specific receptor]; AGMAT [agmatine ureohydrolase (agmatinase)]; AGPS [alkylglycerone phosphate synthase]; AGRN [agrin]; AGRP [agouti related protein homolog (mouse)]; AGT [angiotensinogen (serpin peptidase inhibitor, clade A, member 8)]; AGTR1 [angiotensin II receptor, type 1]; AGTR2 [angiotensin II receptor, type 2]; AHOY [adenosylhomocysteinase]; AH11 [Abelson helper integration site 1]; AHR [aryl hydrocarbon receptor]; AHSP [alpha hemoglobin stabilizing protein]; AICDA [activation-induced cytidine deaminase]; AIDA [axin interactor, dorsalization associated]; AIMP1 [aminoacyl tRNA synthetase complex-interacting multifunctional protein 1]; AIRE [autoimmune regulator]; AK1 [adenylate kinase 1]; AK2 [adenylate kinase 2]; AKR1A1 [aldo-keto reductase family 1, member A1 (aldehyde reductase)]; AKR1B1 [aldo-keto reductase family 1, member B1 (aldose reductase)]; AKR1C3 [aldo-keto reductase family 1, member C3 (3-alpha hydroxysteroid dehydrogenase, type II)]; AKT1 [v-akt murine thymoma viral oncogene homolog 1]; AKT2 [v-akt murine thymoma viral oncogene homolog 2]; AKT3 [v-akt murine thymoma viral oncogene homolog 3 (protein kinase B, gamma)]; ALB [albumin]; ALCAM [activated leukocyte cell adhesion molecule]; ALDHIA1 [aldehyde dehydrogenase 1 family, member A1]; ALDH2 [aldehyde dehydrogenase 2 family (mitochondrial)]; ALDH3A1 [aldehyde dehydrogenase 3 family, memberA1]; ALDH7A1 [aldehyde dehydrogenase 7 family, member A1]; ALDH9A1 [aldehyde dehydrogenase 9 family, member A1]; ALG1 [asparagine-linked glycosylation 1, beta-1,4-mannosyltransferase homolog (S. cerevisiae)]; ALG12 [asparagine-linked glycosylation 12, alpha-1,6-mannosyltransferase homolog (S. cerevisiae)]; ALK [anaplastic lymphoma receptor tyrosine kinase]; ALOX12 [arachidonate 12-lipoxygenase]; ALOX15 [arachidonate 15-lipoxygenase]; ALOX15B [arachidonate 15-lipoxygenase, type B]; ALOX5 [arachidonate 5-lipoxygenase]; ALOX5AP [arachidonate 5-lipoxygenase-activating protein]; ALP1 [alkaline phosphatase, intestinal]; ALPL [alkaline phosphatase, liver/bone/kidney]; ALPP [alkaline phosphatase, placental (Regan isozyme)]; AMACR [alpha-methylacyl-CoA racemase]; AMBP [alpha-1-microglobulin/bikunin precursor]; AMPD3 [adenosine monophosphate deaminase 3]; ANG [angiogenin, ribonuclease, RNase A family, 5]; ANGPT1 [angiopoietin 1]; ANGPT2 [angiopoietin 2]; ANK1 [ankyrin 1, erythrocytic]; ANKH [ankylosis, progressive homolog (mouse)]; ANKRD1 [ankyrin repeat domain 1 (cardiac muscle)]; ANPEP [alanyl (membrane) aminopeptidase]; ANTXR2 [anthrax toxin receptor 2]; ANXA1 [annexin A1]; ANXA2 [annexin A2]; ANXA5 [annexin A5]; ANXA6 [annexin A6]; AOAH [acyloxyacyl hydrolase (neutrophil)]; AOC2 [amine oxidase, copper containing 2 (retina-specific)]; AP2B1 [adaptor-related protein complex 2, beta 1 subunit]; AP3B1 [adaptor-related protein complex 3, beta 1 subunit]; APC [adenomatous polyposis coli]; APCS [amyloid P component, serum]; APEX1 [APEX nuclease (multifunctional DNA repair enzyme) 1]; APLNR [apelin receptor]; APOA1 [apolipoprotein A-1]; APOA2 [apolipoprotein A-II]; APOA4 [apolipoprotein A-IV]; APOB [apolipoprotein B (including Ag(x) antigen)]; APOBEC1 [apolipoprotein B mRNA editing enzyme, catalytic polypeptide 1]; APOBEC3G [apolipoprotein B mRNA editing enzyme, catalytic polypeptide-like 3G]; APOC3 [apolipoprotein C-III]; APOD [apolipoprotcin D]; APOE [apolipoprotcin E]; APOH [apolipoprotcin H (beta-2-glycoprotcin I)]; APP [amyloid beta (A4) precursor protein]; APRT [adenine phosphoribosyltransferase]; APTX [aprataxin]; AQP1 [aquaporin 1 (Colton blood group)]; AQP2 [aquaporin 2 (collecting duct)]; AQP3 [aquaporin 3 (Gill blood group)]; AQP4 [aquaporin 4]; AQP5 [aquaporin 5]; AQP7 [aquaporin 7]; AQP8 [aquaporin 8]; AR [androgen receptor]; AREG [amphiregulin]; ARF6 [ADP-ribosylation factor 6]; ARG1 [arginase, liver]; ARG2 [arginase, type II]; ARHGAP6 [Rho GTPase activating protein 6]; ARHGEF2 [Rho/Rae guanine nucleotide exchange factor (GEF) 2]; ARHGEF6 [Rac/Cdc42 guanine nucleotide exchange factor (GEF) 6]; ARL13B [ADP-ribosylation factor-like 13B]; ARNT [aryl hydrocarbon receptor nuclear translocator]; ARNTL [aryl hydrocarbon receptor nuclear translocator-like]; ARRB1 [arrestin, beta 1]; ARRB2 [arrestin, beta 2]; ARSA [arylsulfatase A]; ARSB [arylsulfatase B]; ARSH [arylsulfatase family, member H]; ART1 [ADP-ribosyltransferase 1]; ASAH1 [N-acylsphingosine amidohydrolase (acid ceramidase) 1]; ASAP1 [ArfGAP with SH3 domain, ankyrin repeat and PH domain 1]; ASGR2 [asialoglycoprotein receptor 2]; ASL [argininosuccinate lyase]; ASNS [asparagine synthetase]; ASPA [aspartoacylase (Canavan disease)]; ASPG [asparaginase homolog (S. cerevisiae)]; ASPH [aspartate beta-hydroxylase]; ASRGL1 [asparaginase like 1]; ASS1 [argininosuccinate synthase 1]; ATF1 [activating transcription factor 1]; ATF2 [activating transcription factor 2]; ATF3 [activating transcription factor 3]; ATF4 [activating transcription factor 4 (tax-responsive enhancer element B67)]; ATG16L1 [ATG16 autophagy related 16-like 1 (S. cerevisiae)]; ATM [ataxia telangiectasia mutated]; ATMIN [ATM interactor]; ATN1 [atrophin 1]; ATOH1 [atonal homolog 1 (Drosophila)]; ATP2A2 [ATPase, Ca++ transporting, cardiac muscle, slow twitch 2]; ATP2A3 [ATPase, Ca++ transporting, ubiquitous]; ATP2C1 [ATPase, Ca++ transporting, type 2C, member 1]; ATP5E [ATP synthase, H+ transporting, mitochondrial F1 complex, epsilon subunit]; ATP7B [ATPase, Cu++ transporting, beta polypeptide]; ATP8B1 [ATPase, class I, type 8B, member 1]; ATPAF2 [ATP synthase mitochondrial F1 complex assembly factor 2]; ATR [ataxia telangiectasia and Rad3 related]; ATRIP [ATR interacting protein]; ATRN [attractin]; AURKA [aurora kinase A]; AURKB [aurora kinase B]; AURKC [aurora kinase C]; AVP [arginine vasopressin]; AVPR2 [arginine vasopressin receptor 2]; AXL [AXL receptor tyrosine kinase]; AZGP1 [alpha-2-glycoprotein 1, zinc-binding]; B2M [beta-2-microglobulin]; B3GALTL [beta 1,3-galactosyltransferase-like]; B3GAT1 [beta-1,3-glucuronyltransferase 1 (glucuronosyltransferase P)]; B4GALNT1 [beta-1,4-N-acetyl-galactosaminyl transferase 1]; B4GALT 1 [UDP-Gal:betaGlcNAc beta 1,4-galactosyltransferase, polypeptide 1]; BACE1 [beta-site APP-cleaving enzyme 1]; BACE2 [beta-site APP-cleaving enzyme 2]; BACH1 [BTB and CNC homology 1, basic leucine zipper transcription factor 1]; BAD [BCL2-associated agonist of cell death]; BAIAP2 [BAI1-associated protein 2]; BAK1 [BCL2-antagonist/killer 1]; BARX2 [BARX homeobox 2]; BAT1 [HLA-B associated transcript 1]; BAT2 [HLA-B associated transcript 2]; BAX [BCL2-associated X protein]; BBC3 [BCL2 binding component 3]; BCAR1 [breast cancer anti-estrogen resistance 1]; BCAT1 [branched chain aminotransferase 1, cytosolic]; BCAT2 [branched chain aminotransferase 2, mitochondrial]; BCHE [butyrylcholinesterase]; BCL10 [B-cell CLL/lymphoma 10]; BCL11B [B-cell CLL/lymphoma 11B (zinc finger protein)]; BCL2 [B-cell CLL/lymphoma 2]; BCL2A1 [BCL2-related protein A1]; BCL2L1 [BCL2-like 1]; BCL2L11 [BCL2-like 11 (apoptosis facilitator)]; BCL3 [B-cell CLL/lymphoma 3]; BCL6 [B-cell CLL/lymphoma 6]; BCR [breakpoint cluster region]; BDKRB1 [bradykinin receptor B]; BDKRB2 [bradykinin receptor B2]; BDNF [brain-derived neurotrophic factor]; BECN1 [beclin 1, autophagy related]; BEST1 [bestrophin 1]; BFAR [bifunctional apoptosis regulator]; BGLAP [bone gamma-carboxyglutamate (gla) protein]; BHMT [betaine-homocysteine methyltransferase]; BID [BH3 interacting domain death agonist]; BIK [BCL2-interacting killer (apoptosis-inducing)]; BIRC2 [baculoviral IAP repeat-containing 2]; BIRC3 [baculoviral IAP repeat-containing 3]; BIRC5 [baculoviral IAP repeat-containing 5]; BLK [B lymphoid tyrosine kinase]; BLM [Bloom syndrome, RecQ helicase-like]; BLNK [B-celllinker]; BLVRB [biliverdin reductase B (flavin reductase (NADPH))J; BMI1 [BMI1 polycomb ring finger oncogene]; BMP1 [bone morphogenetic protein 1]; BMP2 [bone morphogenetic protein 2]; BMP4 [bone morphogenetic protein 4]; BMP6 [bone morphogenetic protein 6]; BMP7 [bone morphogenetic protein 7]; BMPR1A [bone morphogenetic protein receptor, type IA]; BMPR1B [bone morphogenetic protein receptor, type IB]; BMPR2 [bone morphogenetic protein receptor, type II (serine/threonine kinase)]; BPI [bactericidal/permeability-increasing protein]; BRCA1 [breast cancer 1, early onset]; BRCA2 [breast cancer 2, early onset]; BRCC3 [BRCA1/BRCA2-containing complex, subunit 3]; BRD8 [bromodomain containing 8]; BRIP1 [BRCA1 interacting protein C-terminal helicase 1]; BSG [basigin (Ok blood group)]; BSN [bassoon (presynaptic cytomatrix protein)]; BSX [brain-specific homeobox]; BTD [biotinidase]; BTK [Bruton agammaglobulinemia tyrosine kinase]; BTLA [B and T lymphocyte associated]; BTNL2 [butyrophilin-like 2 (MHC class II associated)]; BTRC [beta-transducin repeat containing]; C10orf67 [chromosome 10 open reading frame 67]; C11orf30 [chromosome 11 open reading frame 30]; C11orf58 [chromosome 11 open reading frame 58]; C13orf23 [chromosome 13 open reading frame 23]; C13orf31 [chromosome 13 open reading frame 31]; C15orf2 [chromosome 15 open reading frame 2]; C16orf75 [chromosome 16 open reading frame 75]; C19orf10 [chromosome 19 open reading frame 10]; C1QA [complement component 1, q subcomponent, A chain]; C1QB [complement component 1, q subcomponent, B chain]; C1QC [complement component 1, q subcomponent, C chain]; C1QTNF5 [C1 q and tumor necrosis factor related protein 5]; C1R [complement component 1, r subcomponent]; C1S [complement component 1, s subcomponent]; C2 [complement component 2]; C20orf29 [chromosome 20 open reading frame 29]; C21orf33 [chromosome 21 open reading frame 33]; C3 [complement component 3]; C3AR1 [complement component 3a receptor 1]; C3orf27 [chromosome 3 open reading frame 27]; C4A [complement component 4A (Rodgers blood group)]; C4B [complement component 4B (Chido blood group)]; C4BPA [complement component 4 binding protein, alpha]; C4BPB [complement component 4 binding protein, beta]; C5 [complement component 5]; C5AR1 [complement component 5a receptor 1]; C5orf56 [chromosome 5 open reading frame 56]; C5orf62 [chromosome 5 open reading frame 62]; C6 [complement component 6]; C6orf142 [chromosome 6 open reading frame 142]; C6orf25 [chromosome 6 open reading frame 25]; C7 [complement component 7]; C7orf72 [chromosome 7 open reading frame 72]; C8A [complement component 8, alpha polypeptide]; C8B [complement component 8, beta polypeptide]; C8G [complement component 8, gamma polypeptide]; C8orf38 [chromosome 8 open reading frame 38]; C9 [complement component 9]; CA2 [carbonic anhydrase II]; CA6 [carbonic anhydrase VI]; CA8 [carbonic anhydrase VIII]; CA9 [carbonic anhydrase IX]; CABIN1 [calcineurin binding protein 1]; CACNA1C [calcium channel, voltage-dependent, L type, alpha IC subunit]; CACNA1S [calcium channel, voltage-dependent, L type, alpha 1S subunit]; CAD [carbamoyl-phosphate synthetase 2, aspartate transcarbamylase, and dihydroorotase]; CALB1 [calbindin 1, 28 kDa]; CALB2 [calbindin 2]; CALCA [calcitonin-related polypeptide alpha]; CALCRL [calcitonin receptor-like]; CALD1 [caldesmon 1]; CALM1 [calmodulin 1 (phosphorylase kinase, delta)]; CALM2 [calmodulin 2 (phosphorylase kinase, delta)]; CALM3 [calmodulin 3 (phosphorylase kinase, delta)]; CALR [calreticulin]; CAMK2G [calcium/calmodulin-dependent protein kinase II gamma]; CAMP [cathelicidin antimicrobial peptide]; CANT1 [calcium activated nucleotidase 1]; CANX [calnexin]; CAPN1 [calpain 1, (mu/I) large subunit]; CARD10 [caspase recruitment domain family, member 10]; CARD16 [caspase recruitment domain family, member 16]; CARDS [caspase recruitment domain family, member 8]; CARDS [caspase recruitment domain family, member 9]; CASP1 [caspase 1, apoptosis-related cysteine peptidase (interleukin 1, beta, convertase)]; CASP10 [caspase 10, apoptosis-related cysteine peptidase]; CASP2 [caspase 2, apoptosis-related cysteine peptidase]; CASP3 [caspase 3, apoptosis-related cysteine peptidase]; CASP5 [caspase 5, apoptosis-related cysteine peptidase]; CASP6 [caspase 6, apoptosis-related cysteine peptidase]; CASP7 [caspase 7, apoptosis-related cysteine peptidase]; CASP8 [caspase 8, apoptosis-related cysteine peptidase]; CASP8AP2 [caspase 8 associated protein 2]; CASP9 [caspase 9, apoptosis-related cysteine peptidase]; CASR [calcium-sensing receptor]; CAST [calpastatin]; CAT [catalase]; CAV1 [caveolin 1, caveolae protein, 22 kDa]; CAV2 [caveolin 2]; CBL [Cas-Br-M (murine) ecotropic retroviral transforming sequence]; CBS [cystathionine-beta-synthase]; CBX5 [chromobox homolog 5 (HP1 alpha homolog, Drosophila)]; CC2D2A [coiled-coil and C2 domain containing 2A]; CCBP2 [chemokine binding protein 2]; CCDC144A [coiled-coil domain containing 144A]; CCDC144B [coiled-coil domain containing 144B]; CCDC68 [coiled-coil domain containing 68]; CCK [cholecystokinin]; CCL1 [chemokine (C-C motif) ligand 1]; CCL11 [chemokine (C-C motif) ligand 11]; CCL13 [chemokine (C-C motif) ligand 13]; CCL14 [chemokine (C-C motif) ligand 14]; CCL17 [chemokine (C-C motif) ligand 17]; CCL18 [chemokine (C-C motif) ligand 18 (pulmonary and activation-regulated)]; CCL19 [chemokine (C-C motif) ligand 19]; CCL2 [chemokine (C-C motif) ligand 2]; CCL20 [chemokine (C-C motif) ligand 20]; CCL21 [chemokine (C-C motif) ligand 21]; CCL22 [chemokine (C-C motif) ligand 22]; CCL24 [chemokine (C-C motif) ligand 24]; CCL25 [chemokine (C-C motif) ligand 25]; CCL26 [chemokine (C-C motif) ligand 26]; CCL27 [chemokine (C-C motif) ligand 27]; CCL28 [chemokine (C-C motif) ligand 28]; CCL3 [chemokine (C-C motif) ligand 3]; CCL4 [chemokine (C-C motif) ligand 4]; CCL4L1 [chemokine (C-C motif) ligand 4-like 1]; CCL5 [chemokine (C-C motif) ligand 5]; CCL7 [chemokine (C-C motif) ligand 7]; CCL8 [chemokine (C-C motif) ligand 8]; CCNA1 [cyclin A1]; CCNA2 [cyclin A2]; CCNB1 [cyclin B1]; CCNB2 [cyclin B2]; CCNC [cyclin C]; CCND1 [cyclin D1]; CCND2 [cyclin D2]; CCND3 [cyclin D3]; CCNE1 [cyclin E1]; CCNG1 [cyclin G1]; CCNH [cyclin H]; CCNT1 [cyclin T1]; CCNT2 [cyclin T2]; CCNY [cyclin Y]; CCR1 [chemokine (C-C motif) receptor 1]; CCR2 [chemokine (C-C motif) receptor 2]; CCR3 [chemokine (C-C motif) receptor 3]; CCR4 [chemokine (C-C motif) receptor 4]; CCR5 [chemokine (C-C motif) receptor 5]; CCR6 [chemokine (C-C motif) receptor 6]; CCR7 [chemokine (C-C motif) receptor 7]; CCR8 [chemokine (C-C motif) receptor 8]; CCR9 [chemokine (C-C motif) receptor 9]; CCRL1 [chemokine (C-C motif) receptor-like 1]; CD14 [CD14 molecule]; CD151 [CD151 molecule (Raph blood group)]; CD160 [CD160 molecule]; CD163 [CD163 molecule]; CD180 [CD180 molecule]; CD19 [CD19 molecule]; CD1A [CD1a molecule]; CD1B [CD1b molecule]; CD1C [CD1c molecule]; CD1D [CD1d molecule]; CD2 [CD2 molecule]; CD200 [CD200 molecule]; CD207 [CD207 molecule, langerin]; CD209 [CD209 molecule]; CD22 [CD22 molecule]; CD226 [CD226 molecule]; CD24 [CD24 molecule]; CD244 [CD244 molecule, natural killer cell receptor 2B4]; CD247 [CD247 molecule]; CD27 [CD27 molecule]; CD274 [CD274 molecule]; CD28 [CD28 molecule]; CD2AP [CD2-associated protein]; CD300LF [CD300 molecule-like family member f]; CD34 [CD34 molecule]; CD36 [CD36 molecule (thrombospondin receptor)]; CD37 [CD37 molecule]; CD38 [CD38 molecule]; CD3E [CD3e molecule, epsilon (CD3-TCR complex)]; CD4 [CD4 molecule]; CD40 [CD40 molecule, TNF receptor superfamily member 5]; CD40LG [CD40 ligand]; CD44 [CD44 molecule (Indian blood group)]; CD46 [CD46 molecule, complement regulatory protein]; CD47 [CD47 molecule]; CD48 [CD48 molecule]; CD5 [CD5 molecule]; CD52 [CD52 molecule]; CD53 [CD53 molecule]; CD55 [CD55 molecule, decay accelerating factor for complement (Cromer blood group)]; CD58 [CD58 molecule]; CD59 [CD59 molecule, complement regulatory protein]; CD63 [CD63 molecule]; CD68 [CD68 molecule]; CD69 [CD69 molecule]; CD7 [CD7 molecule]; CD70 [CD70 molecule]; CD72 [CD72 molecule]; CD74 [CD74 molecule, major histocompatibility complex, class II invariant chain]; CD79A [CD79a molecule, immunoglobulin-associated alpha]; CD79B [CD79b molecule, immunoglobulin-associated beta]; CD80 [CD80 molecule]; CD81 [CD81 molecule]; CD82 [CD82 molecule]; CD83 [CD83 molecule]; CD86 [CD86 molecule]; CD8A [CD8a molecule]; CD9 [CD9 molecule]; CD93 [CD93 molecule]; CD97 [CD97 molecule]; CDC20 [cell division cycle 20 homolog (S. cerevisiae)]; CDC25A [cell division cycle 25 homolog A (S. pombe)]; CDC25B [cell division cycle 25 homolog B (S. pombe)]; CDC25C [cell division cycle 25 homolog C (S. pombe)]; CDC42 [cell division cycle 42 (GTP binding protein, 25 kDa)]; CDC45 [CDC45 cell division cycle 45 homolog (S. cerevisiae)]; CDC5L [CDC5 cell division cycle 5-like (S. pombe)]; CDC6 [cell division cycle 6 homolog (S. cerevisiae)]; CDC7 [cell division cycle 7 homolog (S. cerevisiae)]; CDH1 [cadherin 1, type 1, E-cadherin (epithelial)]; CDH2 [cadherin 2, type 1, N-cadherin (neuronal)]; CDH26 [cadherin 26]; CDH3 [cadherin 3, type 1, β-cadherin (placental)]; CDH5 [cadherin 5, type 2 (vascular endothelium)]; CDIPT [CDP-diacylglycerol-inositol 3-phosphatidyltransferase (phosphatidylinositol synthase)]; CDK1 [cyclin-dependent kinase 1]; CDK2 [cyclin-dependent kinase 2]; CDK4 [cyclin-dependent kinase 4]; CDKS [cyclin-dependent kinase 5]; CDKSR1 [cyclin-dependent kinase 5, regulatory subunit 1 (p35)]; CDK7 [cyclin-dependent kinase 7]; CDK9 [cyclin-dependent kinase 9]; CDKAL1 [CDK5 regulatory subunit associated protein 1-like 1]; CDKN1A [cyclin-dependent kinase inhibitor 1A (p21, Cip1)]; CDKN1B [cyclin-dependentkinase inhibitor 1B (p27, Kip1)]; CDKN1C [cyclin-dependent kinase inhibitor IC (p57, Kip2)]; CDKN2A [cyclin-dependent kinase inhibitor 2A (melanoma, p16, inhibits CDK4)]; CDKN2B [cyclin-dependent kinase inhibitor 2B (p15, inhibits CDK4)]; CDKN3 [cyclin-dependent kinase inhibitor 3]; CDR2 [cerebellar degeneration-related protein 2, 62 kDa]; CDT1 [chromatin licensing and DNA replication factor 1]; CDX2 [caudal type homeobox 2]; CEACAM1 [carcinoembryonic antigen-related cell adhesion molecule 1 (biliary glycoprotein)]; CEACAM3 [carcinoembryonic antigen-related cell adhesion molecule 3]; CEACAMS [carcinoembryonic antigen-related cell adhesion molecule 5]; CEACAM6 [carcinoembryonic antigen-related cell adhesion molecule 6 (non-specific cross reacting antigen)]; CEACAM7 [carcinoembryonic antigen-related cell adhesion molecule 7]; CEBPB [CCAAT/enhancer binding protein (C/EBP), beta]; CEL [carboxyl ester lipase (bile salt-stimulated lipase)]; CENPJ [centromere protein J]; CENPV [centromere protein V]; CEP290 [centrosomal protein 290 kDa]; CERK [ceramide kinase]; CETP [cholesteryl ester transfer protein, plasma]; CFB [complement factor B]; CFD [complement factor D (adipsin)]; CFDP1 [craniofacial development protein 1]; CFH [complement factor H]; CFHR1 [complement factor H-related 1]; CFHR3 [complement factor H-related 3]; CFI [complement factor I]; CFL1 [cofilin 1 (non-muscle)]; CFL2 [cofilin 2 (muscle)]; CFLAR [CASP8 and FADD-like apoptosis regulator]; CFP [complement factor properdin]; CFTR [cystic fibrosis transmembrane conductance regulator (ATP-binding cassette sub-family C, member 7)]; CGA [glycoprotein hormones, alpha polypeptide]; CGB [chorionic gonadotropin, beta polypeptide]; CGB5 [chorionic gonadotropin, beta polypeptide 5]; CHAD [chondroadherin]; CHAF1A [chromatin assembly factor 1, subunit A (p150)]; CHAF1B [chromatin assembly factor 1, subunit B (p60)]; CHAT [choline acetyltransferase]; CHD2 [chromodomain helicase DNA binding protein 2]; CHD7 [chromodomain helicase DNA binding protein 7]; CHEK1 [CHK1 checkpoint homolog (S. pombe)]; CHEK2 [CHK2 checkpoint homolog (S. pombe)]; CHGA [chromogranin A (parathyroid secretory protein 1)]; CHGB [chromogranin B (secretogranin 1)]; CHI3L1 [chitinase 3-like 1 (cartilage glycoprotein-39)]; CHIA [chitinase, acidic]; CHIT1 [chitinase 1 (chitotriosidase)]; CHKA [choline kinase alpha]; CHML [choroideremia-like (Rab escort protein 2)]; CHRD [chordin]; CHRDL1 [chordin-like 1]; CHRM1 [cholinergic receptor, muscarinic 1]; CHRM2 [cholinergic receptor, muscarinic 2]; CHRM3 [cholinergic receptor, muscarinic 3]; CHRNA3 [cholinergic receptor, nicotinic, alpha 3]; CHRNA4 [cholinergic receptor, nicotinic, alpha 4]; CHRNA7 [cholinergic receptor, nicotinic, alpha 7]; CHUK [conserved helix-loop-helix ubiquitous kinase]; CIB1 [calcium and integrin binding 1 (calmyrin)]; CIITA [class II, major histocompatibility complex, transactivator]; CILP [cartilage intermediate layer protein, nucleotide pyrophosphohydrolase]; CISH [cytokine inducible SH2-containing protein]; CKB [creatine kinase, brain]; CKLF [chemokine-like factor]; CKM [creatine kinase, muscle]; CLC [Charcot-Leyden crystal protein]; CLCA1 [chloride channel accessory 1]; CLCN1 [chloride channel 1, skeletal muscle]; CLCN3 [chloride channel 3]; CLDN1 [claudin 1]; CLDN11 [claudin 11]; CLDN14 [claudin 14]; CLDN16 [claudin 16]; CLDN19 [claudin 19]; CLDN2 [claudin 2]; CLDN3 [claudin 3]; CLDN4 [claudin 4]; CLDN5 [claudin 5]; CLDN7 [claudin 7]; CLDN8 [claudin 8]; CLEC12A [C-type lectin domain family 12, member A]; CLEC16A [C-type lectin domain family 16, member A]; CLEC4A [C-type lectin domain family 4, member A]; CLEC4D [C-typc lectin domain family 4, member D]; CLEC4M [C-typc lectin domain family 4, member M]; CLEC7A [C-type lectin domain family 7, member A]; CLIP2 [CAP-GLY domain containing linker protein 2]; CLK2 [CDC-like kinase 2]; CLSPN [claspin homolog (Xenopus laevis)]; CLSTN2 [calsyntenin 2]; CLTCL1 [clathrin, heavy chain-like 1]; CLU [clusterin]; CMA1 [chymase 1, mast cell]; CMKLR1 [chemokine-like receptor 1]; CNBP [CCHC-type zinc finger, nucleic acid binding protein]; CNDP2 [CNDP dipeptidase 2 (metallopeptidase M20 family)]; CNN1 [calponin 1, basic, smooth muscle]; CNP [2′,3′-cyclic nucleotide 3′ phosphodiesterase]; CNR1 [cannabinoid receptor 1 (brain)]; CNR2 [cannabinoid receptor 2 (macrophage)]; CNTF [ciliary neurotrophic factor]; CNTN2 [contactin 2 (axonal)]; COG1 [component of oligomeric golgi complex 1]; COG2 [component of oligomeric golgi complex 2]; COIL [coilin]; COL11A1 [collagen, type XI, alpha 1]; COL11A2 [collagen, type XI, alpha 2]; COL17A1 [collagen, type XVII, alpha 1]; COL18A1 [collagen, type XVIII, alpha 1]; COL1A1 [collagen, type I, alpha 1]; COL1A2 [collagen, type I, alpha 2]; COL2A1 [collagen, type II, alpha 1]; COL3A1 [collagen, type III, alpha 1]; COL4A1 [collagen, type IV, alpha 1]; COL4A3 [collagen, type IV, alpha 3 (Goodpasture antigen)]; COL4A4 [collagen, type IV, alpha 4]; COL4A5 [collagen, type IV, alpha 5]; COL4A6 [collagen, type IV, alpha 6]; COL5A1 [collagen, type V, alpha 1]; COL5A2 [collagen, type V, alpha 2]; COL6A1 [collagen, type VI, alpha 1]; COL6A2 [collagen, type VI, alpha 2]; COL6A3 [collagen, type VI, alpha 3]; COL7A1 [collagen, type VII, alpha 1]; COL8A2 [collagen, type VIII, alpha 2]; COL9A1 [collagen, type IX, alpha 1]; COMT [catechol-O-methyltransferase]; COQ3 [coenzyme Q3 homolog, methyltransferase (S. cerevisiae)]; COQ7 [coenzyme Q7 homolog, ubiquinone (yeast)]; CORO1A [coronin, actin binding protein, 1A]; COX10 [COX10 homolog, cytochrome c oxidase assembly protein, heme A: farnesyltransferase (yeast)]; COX15 [COX15 homolog, cytochrome c oxidase assembly protein (yeast)]; COX5A [cytochrome c oxidase subunit Va]; COX8A [cytochrome c oxidase subunit VIIIA (ubiquitous)]; CP [ceruloplasmin (ferroxidase)]; CPA1 [carboxypeptidase A1 (pancreatic)]; CPB2 [carboxypeptidase B2 (plasma)]; CPN1 [carboxypeptidase N, polypeptide 1]; CPOX [coproporphyrinogen oxidase]; CPS1 [carbamoyl-phosphate synthetase 1, mitochondrial]; CPT2 [camitine palmitoyltransferase 2]; CR1 [complement component (3b/4b) receptor 1 (Knops blood group)]; CR2 [complement component (3d/Epstein Barr virus) receptor 2]; CRAT [carnitine O-acetyltransferase]; CRB1 [crumbs homolog 1 (Drosophila)]; CREB1 [cAMP responsive element binding protein 1]; CREBBP [CREB binding protein]; CREM [cAMP responsive element modulator]; CRH [corticotropin releasing hormone]; CRHR1 [cmiicotropin releasing hormone receptor 1]; CRHR2 [corticotropin releasing hormone receptor 2]; CRK [v-crk sarcoma virus CT10 oncogene homolog (avian)]; CRKL [v-crk sarcoma virus CT10 oncogene homolog (avian)-like]; CRLF2 [cytokine receptor-like factor 2]; CRLF3 [cytokine receptor-like factor 3]; CROT [carnitine O-octanoyltransferase]; CRP [C-reactive protein, pentraxin-related]; CRX [cone-rod homeobox]; CRY2 [cryptochrome 2 (photolyase-like)]; CRYAA [crystallin, alpha A]; CRYAB [crystallin, alpha B]; CS [citrate synthase]; CSF1 [colony stimulating factor 1 (macrophage)]; CSF1R [colony stimulating factor 1 receptor]; CSF2 [colony stimulating factor 2 (granulocyte-macrophage)]; CSF2RB [colony stimulating factor 2 receptor, beta, low-affinity (granulocyte-macrophage)]; CSF3 [colony stimulating factor 3 (granulocyte)]; CSF3R [colony stimulating factor 3 receptor (granulocyte)]; CSK [c-src tyrosine kinase]; CSMD3 [CUB and Sushi multiple domains 3]; CSN1S1 [casein alpha sl]; CSN2 [casein beta]; CSNK1A1 [casein kinase 1, alpha 1]; CSNK2A1 [casein kinase 2, alpha 1 polypeptide]; CSNK2B [casein kinase 2, beta polypeptide]; CSPG4 [chondroitin sulfate proteoglycan 4]; CST3 [cystatin C]; CST8 [cystatin 8 (cystatin-related epididymal specific)]; CSTA [cystatin A (stefin A)]; CSTB [cystatin B (stefin B)]; CTAGE1 [cutaneous T-celllymphoma-associated antigen 1]; CTF1 [cardiotrophin 1]; CTGF [connective tissue growth factor]; CTH [cystathionase (cystathionine gamma-lyase)]; CTLA4 [cytotoxic T-lymphocyte-associated protein 4]; CTNNA1 [catenin (cadherin-associated protein), alpha 1, 102 kDa]; CTNNA3 [catenin (cadherin-associated protein), alpha 3]; CTNNAL1 [catenin (cadherin-associated protein), alpha-like 1]; CTNNB1 [catenin (cadherin-associated protein), beta 1, 88 kDa]; CTNND1 [catenin (cadherin-associated protein), delta 1]; CTNS [cystinosis, nephropathic]; CTRL [chymotrypsin-like]; CTSB [cathepsin B]; CTSC [cathepsin C]; CTSD [cathepsin D]; CTSE [cathepsin E]; CTSG [cathepsin G]; CTSH [cathepsin H]; CTSK [cathepsin K]; CTSL1 [cathepsin L]; CTTN [cortactin]; CUL1 [cullin 1]; CUL2 [cullin 2]; CUL4A [cullin 4A]; CULS [cullin 5]; CX3CL1 [chemokine (C-X₃-C motif) ligand 1]; CX3CR1 [chemokine (C-X₃-C motif) receptor 1]; CXADR [coxsackie virus and adenovirus receptor]; CXCL1 [chemokine (C-X-C motif) ligand 1 (melanoma growth stimulating activity, alpha)]; CXCL10 [chemokine (C-X-C motif) ligand 10]; CXCL11 [chemokine (C-X-C motif) ligand 11]; CXCL12 [chemokine (C-X-C motif) ligand 12 (stromal cell-derived factor 1)]; CXCL13 [chemokine (C-X-C motif) ligand 13]; CXCL2 [chemokine (C-X-C motif) ligand 2]; CXCL5 [chemokine (C-X-C motif) ligand 5]; CXCL6 [chemokine (C-X-C motif) ligand 6 (granulocyte chemotactic protein 2)]; CXCL9 [chemokine (C-X-C motif) ligand 9]; CXCR1 [chemokine (C-X-C motif) receptor 1]; CXCR2 [chemokine (C-X-C motif) receptor 2]; CXCR3 [chemokine (C-X-C motif) receptor 3]; CXCR4 [chemokine (C-X-C motif) receptor 4]; CXCR5 [chemokine (C-X-C motif) receptor 5]; CXCR6 [chemokine (C-X-C motif) receptor 6]; CXCR7 [chemokine (C-X-C motif) receptor 7]; CXorf40A [chromosome X open reading frame 40A]; CYB5A [cytochrome b5 type A (microsomal)]; CYB5R3 [cytochrome b5 reductase 3]; CYBA [cytochrome b-245, alpha polypeptide]; CYBB [cytochrome b-245, beta polypeptide]; CYC1 [cytochrome c-1]; CYCS [cytochrome c, somatic]; CYFIP2 [cytoplasmic FMR1 interacting protein 2]; CYP11A1 [cytochrome P450, family 11, subfamily A, polypeptide 1]; CYP11B1 [cytochrome P450, family 11, subfamily B, polypeptide 1]; CYP11B2 [cytochrome P450, family 11, subfamily B, polypeptide 2]; CYP17A1 [cytochrome P450, family 17, subfamily A, polypeptide 1]; CYP19A1 [cytochrome P450, family 19, subfamily A, polypeptide 1]; CYP1A1 [cytochrome P450, family 1, subfamily A, polypeptide 1]; CYP1A2 [cytochrome P450, family 1, subfamily A, polypeptide 2]; CYP1B1 [cytochrome P450, family 1, subfamily B, polypeptide 1]; CYP21A2 [cytochrome P450, family 21, subfamily A, polypeptide 2]; CYP24A1 [cytochrome P450, family 24, subfamily A, polypeptide 1]; CYP27A1 [cytochrome P450, family 27, subfamily A, polypeptide 1]; CYP27B1 [cytochrome P450, family 27, subfamily B, polypeptide 1]; CYP2A6 [cytochrome P450, family 2, subfamily A, polypeptide 6]; CYP2B6 [cytochrome P450, family 2, subfamily B, polypeptide 6]; CYP2C19 [cytochrome P450, family 2, subfamily C, polypeptide 19]; CYP2C8 [cytochrome P450, family 2, subfamily C, polypeptide 8]; CYP2C9 [cytochrome P450, family 2, subfamily C, polypeptide 9]; CYP2D6 [cytochrome P450, family 2, subfamily D, polypeptide 6]; CYP2E1 [cytochrome P450, family 2, subfamily E, polypeptide 1]; CYP2J2 [cytochrome P450, family 2, subfamily J, polypeptide 2]; CYP2R1 [cytochrome P450, family 2, subfamily R, polypeptide 1]; CYP3A4 [cytochrome P450, family 3, subfamily A, polypeptide 4]; CYP3A5 [cytochrome P450, family 3, subfamily A, polypeptide 5]; CYP4F3 [cytochrome P450, family 4, subfamily F, polypeptide 3]; CYP51A1 [cytochrome P450, family 51, subfamily A, polypeptide 1]; CYP7A1 [cytochrome P450, family 7, subfamily A, polypeptide 1]; CYR61 [cysteine-rich, angiogenic inducer, 61]; CYSLTR1 [cysteinyl leukotriene receptor 1]; CYSLTR2 [cysteinylleukotriene receptor 2]; DAO [D-amino-acid oxidase]; DAOA [D-amino acid oxidase activator]; DAP3 [death associated protein 3]; DAPK1 [death-associated protein kinase 1]; DARC [Duffy blood group, chemokine receptor]; DAZ1 [deleted in azoospermia 1]; DBH [dopamine beta-hydroxylase (dopamine beta-monooxygenase)]; DCK [deoxycytidine kinase]; DCLRE1C [DNA cross-link repair IC (PS02 homolog, S. cerevisiae)]; DCN [decorin]; DCT [dopachrome tautomerase (dopachrome delta-isomerase, tyrosine-related protein 2)]; DCTN2 [dynactin 2 (p50)]; DDB1 [damage-specific DNA binding protein 1, 127 kDa]; DDB2 [damage-specific DNA binding protein 2, 48 kDa]; DDC [dopa decarboxylase (aromatic L-amino acid decarboxylase)]; DDIT3 [DNA-damage-inducible transcript 3]; DDR1 [discoidin domain receptor tyrosine kinase 1]; DDX1 [DEAD (Asp-Glu-Ala-Asp) (SEQ ID NO: 532) box polypeptide 1]; DDX41 [DEAD (Asp-Glu-Ala-Asp) (SEQ ID NO: 532) box polypeptide 41]; DDX42 [DEAD (Asp-Glu-Ala-Asp) (SEQ ID NO: 532) box polypeptide 42]; DDX58 [DEAD (Asp-Glu-Ala-Asp) (SEQ ID NO: 532) box polypeptide 58]; DEFA1 [defensin, alpha 1]; DEFAS [defensin, alpha 5, Paneth cell-specific]; DEFA6 [defensin, alpha 6, Paneth cell-specific]; DEFB1 [defensin, beta 1]; DEFB103B [defensin, beta 103B]; DEFB104A [defensin, beta 104A]; DEFB4A [defensin, beta 4A]; DEK [DEK oncogene]; DENND1B [DENN/MADD domain containing IB]; DES [desmin]; DGAT1 [diacylglycerol O-acyltransferase homolog 1 (mouse)]; DGCR14 [DiGeorge syndrome critical region gene 14]; DGCR2 [DiGeorge syndrome critical region gene 2]; DGCR6 [DiGeorge syndrome critical region gene 6]; DGCR6L [DiGeorge syndrome critical region gene 6-like]; DGCR8 [DiGeorge syndrome critical region gene 8]; DGUOK [deoxyguanosine kinase]; DHFR [dihydrofolate reductase]; DHODH [dihydroorotate dehydrogenase]; DHPS [deoxyhypusine synthase]; DHRS7B [dehydrogenase/reductase (SDR family) member 7B]; DHRS9 [dehydrogenase/reductase (SDR family) member 9]; DIAPH1 [diaphanous homolog 1 (Drosophila)]; DICER1 [dicer 1, ribonuclease type III]; DI02 [deiodinase, iodothyronine, type II]; DKC1 [dyskeratosis congenita 1, dyskerin]; DKK1 [dickkopf homolog 1 (Xenopus laevis)]; DLAT [dihydrolipoamide S-acetyltransferase]; DLG2 [discs, large homolog 2 (Drosophila)]; DLG5 [discs, large homolog 5 (Drosophila)]; DMBT1 [deleted in malignant brain tumors 1]; DMC1 [DMC1 dosage suppressor of mckl homolog, meiosis-specific homologous recombination (yeast)]; DMD [dystrophin]; DMP1 [dentin matrix acidic phosphoprotein 1]; DMPK [dystrophia myotonica-protein kinase]; DMRT1 [doublesex and mab-3 related transcription factor 1]; DMXL2 [Dmx-like 2]; DNA2 [DNA replication helicase 2 homolog (yeast)]; DNAH1 [dynein, axonemal, heavy chain 1]; DNAH12 [dynein, axonemal, heavy chain 12]; DNAI1 [dynein, axonemal, intermediate chain 1]; DNAI2 [dynein, axonemal, intermediate chain 2]; DNASE1 [deoxyribonuclease I]; DNM2 [dynamin 2]; DNM3 [dynamin 3]; DNMT1 [DNA (cytosine-5-)-methyltransferase 1]; DNMT3B [DNA (cytosine-5-)-methyltransferase 3 beta]; DNTT [deoxynucleotidyltransferase, terminal]; DOCK1 [dedicator of cytokinesis 1]; DOCK3 [dedicator of cytokinesis 3]; DOCK8 [dedicator of cytokinesis 8]; DOK1 [docking protein 1, 62 kDa (downstream of tyrosine kinase 1)]; DOLK [dolichol kinase]; DPAGT1 [dolichyl-phosphate (UDP-N-acetylglucosamine) N-acetylglucosaminephosphotransferase 1 (GlcNAc-1-P transferase)]; DPEP1 [dipeptidase 1 (renal)]; DPH1 [DPH1 homolog (S. cerevisiae)]; DPM1 [dolichyl-phosphate mannosyltransferase polypeptide 1, catalytic subunit]; DPP10 [dipeptidyl-peptidase 10]; DPP4 [dipeptidyl-peptidase 4]; DPYD [dihydropyrimidine dehydrogenase]; DRD2 [dopamine receptor D2]; DRD3 [dopamine receptor D3]; DRD4 [dopamine receptor D4]; DSC2 [desmocollin 2]; DSG1 [desmoglein 1]; DSG2 [desmoglein 2]; DSG3 [desmoglein 3 (pemphigus vulgaris antigen)]; DSP [desmoplakin]; DTNA [dystrobrevin, alpha]; DTYMK [deoxythymidylate kinase (thymidylate kinase)]; DUOX1 [dual oxidase 1]; DUOX2 [dual oxidase 2]; DUSP1 [dual specificity phosphatase 1]; DUSP14 [dual specificity phosphatase 14]; DUSP2 [dual specificity phosphatase 2]; DUSP5 [dual specificity phosphatase 5]; DUT [deoxyuridine triphosphatase]; DVL1 [dishevelled, dsh homolog 1 (Drosophila)]; DYNC2H1 [dynein, cytoplasmic 2, heavy chain 1]; DYNLL1 [dynein, light chain, LC8-type 1]; DYRKIA [dual-specificity tyrosine-(Y)-phosphmylation regulated kinase 1A]; DYSF [dysferlin, limb girdle muscular dystrophy 2B (autosomal recessive)]; E2F1 [E2F transcription factor 1]; EBF2 [early B-cell factor 2]; EB13 [Epstein-Barr virus induced 3]; ECE1 [endothelin converting enzyme 1]; ECM1 [extracellular matrix protein 1]; EDA [ectodysplasin A]; EDAR [ectodysplasin A receptor]; EDN1 [endothelin 1]; EDNRA [endothelin receptor type A]; EDNRB [endothelin receptor type B]; EEF1A1 [eukaryotic translation elongation factor 1 alpha 1]; EEF1A2 [eukaryotic translation elongation factor 1 alpha 2]; EFEMP2 [EGF-containing fibulin-like extracellular matrix protein 2]; EFNA1 [ephrin-A1]; EFNB2 [ephrin-B2]; EFS [embryonal Fyn-associated substrate]; EGF [epidermal growth factor (beta-urogastrone)]; EGFR [epidermal growth factor receptor (erythroblastic leukemia viral (v-erb-b) oncogene homolog, avian)]; EGR1 [early growth response 1]; EGR2 [early growth response 2]; EHF [ets homologous factor]; EHMT2 [euchromatic histone-lysine N-methyltransferase 2]; EIF2AK2 [eukaryotic translation initiation factor 2-alpha kinase 2]; EIF2S1 [eukaryotic translation initiation factor 2, subunit 1 alpha, 35 kDa]; EIF2S2 [eukaryotic translation initiation factor 2, subunit 2 beta, 38 kDa]; EIF3A [eukaryotic translation initiation factor 3, subunit A]; EIF4B [eukaryotic translation initiation factor 4B]; EIF4E [eukaryotic translation initiation factor 4E]; EIF4EBP1 [eukaryotic translation initiation factor 4E binding protein 1]; EIF4G1 [eukaryotic translation initiation factor 4 gamma, 1]; EIF6 [eukaryotic translation initiation factor 6]; ELAC2 [elaC homolog 2 (E. coli)]; ELANE [elastase, neutrophil expressed]; ELAVL1 [ELAV (embryonic lethal, abnormal vision, Drosophila)-like 1 (Hu antigen R)]; ELF3 [E74-like factor 3 (ets domain transcription factor, epithelial-specific)]; ELF5 [E74-like factor 5 (ets domain transcription factor)]; ELN [elastin]; ELOVL4 [elongation of very long chain fatty acids (FEN1/Elo2, SUR4/Elo3, yeast)-like 4]; EMD [emerin]; EMILIN1 [elastin microfibril interfacer 1]; EMR2 [egf-like module containing, mucin-like, hormone receptor-like 2]; EN2 [engrailed homeobox 2]; ENG [endoglin]; ENO1 [enolase 1, (alpha)]; ENO2 [enolase 2 (gamma, neuronal)]; ENO3 [enolase 3 (beta, muscle)]; ENPP2 [ectonucleotide pyrophosphatase/phosphodiesterase 2]; ENPP3 [ectonucleotide pyrophosphatase/phosphodiesterase 3]; ENTPD1 [ectonucleoside triphosphate diphosphohydrolase 1]; EP300 [E1A binding protein p300]; EPAS1 [endothelial PAS domain protein 1]; EPB42 [erythrocyte membrane protein band 4.2]; EPCAM [epithelial cell adhesion molecule]; EPHA1 [EPH receptor A1]; EPHA2 [EPH receptor A2]; EPHB2 [EPH receptor B2]; EPHB4 [EPH receptor B4]; EPHB6 [EPH receptor B6]; EPHX1 [epoxide hydrolase 1, microsomal (xenobiotic)]; EPHX2 [epoxide hydrolase 2, cytoplasmic]; EPO [erythropoietin]; EPOR [erythropoietin receptor]; EPRS [glutamyl-prolyl-tRNA synthetase]; EPX [eosinophil peroxidase]; ERBB2 [v-erb-b2 erythroblastic leukemia viral oncogene homolog 2, neuro/glioblastoma derived oncogene homolog (avian)]; ERBB21P [erbb2 interacting protein]; ERBB3 [v-erb-b2 erythroblastic leukemia viral oncogene homolog 3 (avian)]; ERBB4 [v-erb-a erythroblastic leukemia viral oncogene homolog 4 (avian)]; ERCC1 [excision repair cross-complementing rodent repair deficiency, complementation group 1 (includes overlapping antisense sequence)]; ERCC2 [excision repair cross-complementing rodent repair deficiency, complementation group 2]; ERCC3 [excision repair cross-complementing rodent repair deficiency, complementation group 3 (xeroderma pigmentosum group B complementing)]; ERCC4 [excision repair cross-complementing rodent repair deficiency, complementation group 4]; ERCC5 [excision repair cross-complementing rodent repair deficiency, complementation group 5]; ERCC6 [excision repair cross-complementing rodent repair deficiency, complementation group 6]; ERCC6L [excision repair cross-complementing rodent repair deficiency, complementation group 6-like]; ERCC8 [excision repair cross-complementing rodent repair deficiency, complementation group 8]; ERO1LB [ERO1-like beta (S. cerevisiae)]; ERVK6 [endogenous retroviral sequence K, 6]; ERVWE1 [endogenous retroviral family W, env(C7), member 1]; ESD [esterase D/formylglutathione hydrolase]; ESR1 [estrogen receptor 1]; ESR2 [estrogen receptor 2 (ER beta)]; ESRRA [estrogen-related receptor alpha]; ESRRB [estrogen-related receptor beta]; ETS1 [v-ets erythroblastosis virus E26 oncogene homolog 1 (avian)]; ETS2 [v-ets erythroblastosis virus E26 oncogene homolog 2 (avian)]; EWSR1 [Ewing sarcoma breakpoint region 1]; EX01 [exonuclease 1]; EYA1 [eyes absent homolog 1 (Drosophila)]; EZH2 [enhancer ofzeste homolog 2 (Drosophila)]; EZR [ezrin]; F10 [coagulation factor X]; F11 [coagulation factor XI]; F12 [coagulation factor XII (Hageman factor)]; F13A1 [coagulation factor XIII, A1 polypeptide]; F13B [coagulation factor XIII, B polypeptide]; F2 [coagulation factor II (thrombin)]; F2R [coagulation factor II (thrombin) receptor]; F2RL1 [coagulation factor II (thrombin) receptor-like 1]; F2RL3 [coagulation factor II (thrombin) receptor-like 3]; F3 [coagulation factor III (thromboplastin, tissue factor)]; F5 [coagulation factor V (proaccelerin, labile factor)]; F7 [coagulation factor VII (serum prothrombin conversion accelerator)]; F8 [coagulation factor VIII, procoagulant component]; F9 [coagulation factor IX]; FABP1 [fatty acid binding protein 1, liver]; FABP2 [fatty acid binding protein 2, intestinal]; FABP4 [fatty acid binding protein 4, adipocyte]; FADD [Fas (TNFRSF6)-associated via death domain]; FADS1 [fatty acid desaturase 1]; FADS2 [fatty acid desaturase 2]; FAF1 [Fas (TNFRSF6) associated factor 1]; FAH [fumarylacetoacctate hydrolase (fumarylacetoacctase)]; FAM189B [family with sequence similarity 189, member B]; FAM92B [family with sequence similarity 92, member B]; FANCA [Fanconi anemia, complementation group A]; FANCB [Fanconi anemia, complementation group B]; FANCC [Fanconi anemia, complementation group C]; FANCD2 [Fanconi anemia, complementation group D2]; FANCE [Fanconi anemia, complementation groupE]; FANCF [Fanconi anemia, complementation group F]; FANCG [Fanconi anemia, complementation group G]; FANGI [Fanconi anemia, complementation group I]; FANCL [Fanconi anemia, complementation group L]; FANCM [Fanconi anemia, complementation group M]; FANK1 [fibronectin type III and ankyrin repeat domains 1]; FAS [Fas (TNF receptor superfamily, member 6)]; FASLG [Fas ligand (TNF superfamily, member 6)]; FASN [fatty acid synthase]; FASTK [Pas-activated serine/threonine kinase]; FBLN5 [fibulin 5]; FBN1 [fibrillin 1]; FBP1 [fructose-1,6-bisphosphatase 1]; FBX032 [F-box protein 32]; FBXW7 [F-box and WD repeat domain containing 7]; FCAR [Fe fragment of IgA, receptor for]; FCER1A [Fc fragment of IgE, high affinity I, receptor for; alpha polypeptide]; FCER1G [Fc fragment of IgE, high affinity I, receptor for; gamma polypeptide]; FCER2 [Fc fragment of IgE, low affinity II, receptor for (CD23)]; FCGR1A [Fc fragment of IgG, high affinity Ia, receptor (CD64)]; FCGR2A [Fc fragment of IgG, low affinity IIa, receptor (CD32)]; FCGR2B [Fc fragment of IgG, low affinity 11b, receptor (CD32)]; FCGR3A [Fc fragment of IgG, low affinity IIIa, receptor (CD16a)]; FCGR3B [Fc fragment of IgG, low affinity IIIb, receptor (CD16b)]; FCN2 [ficolin (collagen/fibrinogen domain containing lectin) 2 (hucolin)]; FCN3 [ficolin (collagen/fibrinogen domain containing) 3 (Hakata antigen)]; FCRL3 [Fc receptor-like 3]; FCRL6 [Fc receptor-like 6]; FDFT1 [farnesyl-diphosphate farnesyltransferase 1]; FDPS [farnesyl diphosphate synthase (farnesyl pyrophosphate synthetase, dimethylallyltranstransferase, geranyltranstransferase)]; FDX1 [ferredoxin 1]; FEN1 [flap structure-specific endonuclease 1]; FERMT1 [fermitin family homolog 1 (Drosophila)]; FERMT3 [fermitin family homolog 3 (Drosophila)]; FES [feline sarcoma oncogene]; FFAR2 [free fatty acid receptor 2]; FGA [fibrinogen alpha chain]; FGB [fibrinogen beta chain]; FGF1 [fibroblast growth factor 1 (acidic)]; FGF2 [fibroblast growth factor 2 (basic)]; FGF5 [fibroblast growth factor 5]; FGF7 [fibroblast growth factor 7 (keratinocyte growth factor)]; FGF8 [fibroblast growth factor 8 (androgen-induced)]; FGFBP2 [fibroblast growth factor binding protein 2]; FGFR1 [fibroblast growth factor receptor 1]; FGFR10P [FGFR1 oncogene partner]; FGFR2 [fibroblast growth factor receptor 2]; FGFR3 [fibroblast growth factor receptor 3]; FGFR4 [fibroblast growth factor receptor 4]; FGG [fibrinogen gamma chain]; FGR [Gardner-Rasheed feline sarcoma viral (v-fgr) oncogene homolog]; FHIT [fragile histidine triad gene]; FHL1 [four and a half LIM domains 1]; FHL2 [four and a half LIM domains 2]; FIBP [fibroblast growth factor (acidic) intracellular binding protein]; FIGF [c-fos induced growth factor (vascular endothelial growth factor D)]; FKBP1A [FK506 binding protein 1A, 12 kDa]; FKBP4 [FK506 binding protein 4, 59 kDa]; FKBP5 [FK506 binding protein 5]; FLCN [folliculin]; FLG [filaggrin]; FLG2 [filaggrin family member 2]; FLNA [filamin A, alpha]; FLNB [filamin B, beta]; FLT1 [fins-related tyrosine kinase 1 (vascular endothelial growth factor/vascular permeability factor receptor)]; FLT3 [fms-related tyrosine kinase 3]; FLT3LG [fms-related tyrosine kinase 3 ligand]; FLT4 [fms-related tyrosine kinase 4]; FMN1 [formin 1]; FMOD [fibromodulin]; FMR1 [fragile X mental retardation 1]; FN1 [fibronectin 1]; FOLH1 [folate hydrolase (prostate-specific membrane antigen) 1]; FOLR1 [folate receptor 1 (adult)]; FOS [FBJ murine osteosarcoma viral oncogene homolog]; FOXL2 [forkhead box L2]; FOXN1 [forkhead box N1]; FOXN2 [forkhead box N2]; FOXO3 [forkhead box O3]; FOXP3 [forkhead box P3]; FPGS [folylpolyglutamate synthase]; FPR1 [formyl peptide receptor 1]; FPR2 [formyl peptide receptor 2]; FRAS1 [Fraser syndrome 1]; FREM2 [FRAS1 related extracellular matrix protein 2]; FSCN1 [fascin homolog 1, actin-bundling protein (Strongylocentrotus purpuratus)]; FSHB [follicle stimulating hormone, beta polypeptide]; FSHR [follicle stimulating hormone receptor]; FST [follistatin]; FTCD [formiminotransferase cyclodeaminase]; FTH1 [ferritin, heavy polypeptide 1]; FTL [ferritin, light polypeptide]; FURIN [furin (paired basic amino acid cleaving enzyme)]; FUT1 [fucosyltransferase 1 (galactoside 2-alpha-L-fucosyltransferase, H blood group)]; FUT2 [fucosyltransferase 2 (secretor status included)]; FUT3 [fucosyltransferase 3 (galactoside 3(4)-L-fucosyltransferase, Lewis blood group)]; FUT4 [fucosyltransferase 4 (alpha (1,3) fucosyltransferase, myeloid-specific)]; FUT7 [fucosyltransferase 7 (alpha (1,3) fucosyltransferase)]; FUT8 [fucosyltransferase 8 (alpha (1,6) fucosyltransferase)]; FXN [frataxin]; FYN [FYN oncogene related to SRC, FGR, YES]; FZD4 [frizzled homolog 4 (Drosophila)]; G6PC3 [glucose 6 phosphatase, catalytic, 3]; G6PD [glucose-6-phosphate dehydrogenase]; GAA [glucosidase, alpha; acid]; GAB2 [GRB2-associated binding protein 2]; GABBR1 [gamma-aminobutyric acid (GABA) B receptor, 1]; GABRB3 [gamma-aminobutyric acid (GABA) A receptor, beta 3]; GABRE [gamma-aminobutyric acid (GABA) A receptor, epsilon]; GAD1 [glutamate decarboxylase 1 (brain, 67 kDa)]; GAD2 [glutamate decarboxylase 2 (pancreatic islets and brain, 65 kDa)]; GADD45A [growth arrest and DNA-damage-inducible, alpha]; GAL [galanin prepropeptide]; GALC [galactosylceramidase]; GALK1 [galactokinase 1]; GALR1 [galanin receptor 1]; GAP43 [growth associated protein 43]; GAPDH [glyceraldehyde-3-phosphate dehydrogenase]; GART [phosphoribosylglycinamide formyltransferase, phosphoribosylglycinamide synthetase, phosphoribosylaminoimidazole synthetase]; GAST [gastrin]; GATA1 [GATA binding protein 1 (globin transcription factor 1)]; GATA2 [GATA binding protein 2]; GATA3 [GATA binding protein 3]; GATA4 [GATA binding protein 4]; GATA6 [GATA binding protein 6]; GBA [glucosidase, beta, acid]; GBA3 [glucosidase, beta, acid 3 (cytosolic)]; GBE1 [glucan (1 [4-alpha-), branching enzyme 1]; GC [group-specific component (vitamin D binding protein)]; GCG [glucagon]; GCH1 [GTP cyclohydrolase 1]; GCKR [glucokinase (hexokinase 4) regulator]; GCLC [glutamate-cysteine ligase, catalytic subunit]; GCLM [glutamate-cysteine ligase, modifier subunit]; GCNT2 [glucosaminyl (N-acetyl) transferase 2, 1-branching enzyme (I blood group)]; GDAP1 [ganglioside-induced differentiation-associated protein 1]; GDF15 [growth differentiation factor 15]; GDNF [glial cell derived neurotrophic factor]; GFAP [glial fibrillary acidic protein]; GGH [gamma-glutamyl hydrolase (conjugase, folylpolygammaglutamyl hydrolase)]; GGT1 [gamma-glutamyltransferase 1]; GGT2 [gamma-glutamyltransferase 2]; GH1 [growth hormone 1]; GHR [growth hormone receptor]; GHRH [growth hormone releasing hormone]; GHRL [ghrelin/obestatin prepropeptide]; GHSR [growth hormone secretagogue receptor]; GIF [gastric intrinsic factor (vitamin B synthesis)]; GIP [gastric inhibitory polypeptide]; GJA1 [gap junction protein, alpha 1, 43 kDa]; GJA4 [gap junction protein, alpha 4, 37 kDa]; GJB2 [gap junction protein, beta 2, 26 kDa]; GLA [galactosidase, alpha]; GLB1 [galactosidase, beta 1]; GLI2 [GLI family zinc finger 2]; GLMN [glomulin, FKBP associated protein]; GLX [glutaredoxin (thioltransferase)]; GLS [glutaminase]; GLT25D1 [glycosyltransferase 25 domain containing 1]; GLUL [glutamate-ammonia ligase (glutamine synthetase)]; GLYAT [glycine-N-acyltransferase]; GM2A [GM2 ganglioside activator]; GMDS [GDP-mannose 4 [6-dehydratase]; GNA12 [guanine nucleotide binding protein (G protein) alpha 12]; GNA13 [guanine nucleotide binding protein (G protein), alpha 13]; GNA11 [guanine nucleotide binding protein (G protein), alpha inhibiting activity polypeptide 1]; GNAO1 [guanine nucleotide binding protein (G protein), alpha activating activity polypeptide 0]; GNAQ [guanine nucleotide binding protein (G protein), q polypeptide]; GNAS [GNAS complex locus]; GNAZ [guanine nucleotide binding protein (G protein), alpha z polypeptide]; GNB1 [guanine nucleotide binding protein (G protein), beta polypeptide 1]; GNB lL [guanine nucleotide binding protein (G protein), beta polypeptide 1-like]; GNB2L1 [guanine nucleotide binding protein (G protein), beta polypeptide 2-like 1]; GNB3 [guanine nucleotide binding protein (G protein), beta polypeptide 3]; GNE [glucosamine (UDP-N-acetyl)-2-epimerase/N-acetylmannosamine kinase]; GNG2 [guanine nucleotide binding protein (G protein), gamma 2]; GNLY [granulysin]; GNPAT [glyceronephosphate O-acyltransferase]; GNPDA2 [glucosamine-6-phosphate deaminase 2]; GNRH1 [gonadotropin-releasing hormone 1 (luteinizing-releasing hormone)]; GNRHR [gonadotropin-releasing hormone receptor]; GOLGA8B [golgin A8 family, member B]; GOLGB1 [golgin B1]; GOT1 [glutamic-oxaloacetic transaminase 1, soluble (aspartate aminotransferase 1)]; GOT2 [glutamic-oxaloacetic transaminase 2, mitochondrial (aspartate aminotransferase 2)]; GP1BA [glycoprotein Ib (platelet), alpha polypeptide]; GP2 [glycoprotein 2 (zymogen granule membrane)]; GP6 [glycoprotein VI (platelet)]; GPBAR1 [G protein-coupled bile acid receptor 1]; GPC5 [glypican 5]; GPI [glucose phosphate isomerase]; GPLD1 [glycosylphosphatidylinositol specific phospholipase D1]; GPN1 [GPN-loop GTPase 1]; GPR1 [G protein-coupled receptor 1]; GPR12 [G protein-coupled receptor 12]; GPR123 [G protein-coupled receptor 123]; GPR143 [G protein-coupled receptor 143]; GPR15 [G protein-coupled receptor 15]; GPR182 [G protein-coupled receptor 182]; GPR44 [G protein-coupled receptor 44]; GPR77 [G protein-coupled receptor 77]; GPRASP1 [G protein-coupled receptor associated sorting protein 1]; GPRC6A [G protein-coupled receptor, family C, group 6, member A]; GPT [glutamic-pyruvate transaminase (alanine aminotransferase)]; GPX1 [glutathione peroxidase 1]; GPX2 [glutathione peroxidase 2 (gastrointestinal)]; GPX3 [glutathione peroxidase 3 (plasma)]; GRAP2 [GRB2-related adaptor protein 2]; GRB2 [growth factor receptor-bound protein 2]; GRIA2 [glutamate receptor, ionotropic, AMPA 2]; GRIN1 [glutamate receptor, ionotropic, N-methyl D-aspartate 1]; GRIN2A [glutamate receptor, ionotropic, N-methyl D-aspartate 2A]; GRIN2B [glutamate receptor, ionotropic, N-methyl D-aspartate 2B]; GRIN2C [glutamate receptor, ionotropic, N-methyl D-aspartate 20]; GRIN2D [glutamate receptor, ionotropic, N-methyl D-aspartate 2D]; GRIN3A [glutamate receptor, ionotropic, N-methyl-D-aspartate 3A]; GRIN3B [glutamate receptor, ionotropic, N-methyl-D-aspartate 3B]; GRK5 [G protein-coupled receptor kinase 5]; GRLF1 [glucocorticoid receptor DNA binding factor 1]; GRM1 [glutamate receptor, metabotropic 1]; GRP [gastrin-releasing peptide]; GRPR [gastrin-releasing peptide receptor]; GSC [goosecoid homeobox]; GSC2 [goosecoid homeobox 2]; GSDMB [gasdermin B]; GSK3B [glycogen synthase kinase 3 beta]; GSN [gelsolin]; GSR [glutathione reductase]; GSS [glutathione synthetase]; GSTA1 [glutathione S-transferase alpha 1]; GSTA2 [glutathione S-transferase alpha 2]; GSTM1 [glutathione S-transferase mu 1]; GSTM3 [glutathione S-transferase mu 3 (brain)]; GST02 [glutathione S-transferase omega 2]; GSTP1 [glutathione S-transferase pi 1]; GSTT1 [glutathione S-transferase theta 1]; GTF2A1 [general transcription factor IIA, 1, 19/37 kDa]; GTF2F1 [general transcription factor IIF, polypeptide 1, 74 kDa]; GTF2H2 [general transcription factor IIH, polypeptide 2, 44 kDa]; GTF2H4 [general transcription factor IIH, polypeptide 4, 52 kDa]; GTF2H5 [general transcription factor IIH, polypeptide 5]; GTF2I [general transcription factor IIi]; GTF3A [general transcription factor 111A]; GUCA2A [guanylate cyclase activator 2A (guanylin)]; GUCA2B [guanylate cyclase activator 2B (uroguanylin)]; GUCY2C [guanylate cyclase 2C (heat stable enterotoxin receptor)]; GUK1 [guanylate kinase 1]; GULP1 [GULP, engulfment adaptor PTB domain containing 1]; GUSB [glucuronidase, beta]; GYPA [glycophorin A (MNS blood group)]; GYPB [glycophorin B (MNS blood group)]; GYPC [glycophorin C (Gerbich blood group)]; GYPE [glycophorin E (MNS blood group)]; GYS1 [glycogen synthase 1 (muscle)]; GZMA [granzyme A (granzyme 1, cytotoxic T-lymphocyte-associated serine esterase 3)]; GZMB [granzyme B (granzyme 2, cytotoxic T-lymphocyte-associated serine esterase 1)]; GZMK [granzyme K (granzyme 3; tryptase II)]; H1F0 [H1 histone family, member 0]; H2AFX [H2A histone family, member X]; HABP2 [hyaluronan binding protein 2]; HACL1 [2-hydroxyacyl-CoA lyase 1]; HADHA [hydroxyacyl-Coenzyme A dehydrogenase/3-ketoacyl-Coenzyme A thiolase/enoyl-Coenzyme A hydratase (trifunctional protein), alpha subunit]; HAL [histidine ammonia-lyase]; HAMP [hepcidin antimicrobial peptide]; HAPLN1 [hyaluronan and proteoglycan link protein 1]; HAVCR1 [hepatitis A virus cellular receptor 1]; HAVCR2 [hepatitis A virus cellular receptor 2]; HAX1 [HCLS1 associated protein X-1]; HBA1 [hemoglobin, alpha 1]; HBA2 [hemoglobin, alpha 2]; HBB [hemoglobin, beta]; HBE1 [hemoglobin, epsilon 1]; HBEGF [heparin-binding EGF-Iike growth factor]; HBG2 [hemoglobin, gamma G]; HCCS [holocytochrome c synthase (cytochrome c heme-lyase)]; HCK [hemopoietic cell kinase]; HCRT [hypocretin (orexin) neuropeptide precursor]; HCRTR1 [hypocretin (orexin) receptor 1]; HCRTR2 [hypocretin (orexin) receptor 2]; HOST [hematopoietic cell signal transducer]; HDAC1 [histone deacetylase 1]; HDAC2 [histone deacetylase 2]; HDAC6 [histone deacetylase 6]; HDAC9 [histone deacetylase 9]; HOC [histidine decarboxylase]; HERC2 [hect domain and RLD 2]; HES1 [hairy and enhancer of split 1, (Drosophila)]; HES6 [hairy and enhancer of split 6 (Drosophila)]; HESX1 [HESX homeobox 1]; HEXA [hexosaminidase A (alpha polypeptide)]; HEXB [hexosaminidase B (beta polypeptide)]; HFE [hemochromatosis]; HGF [hepatocyte growth factor (hepapoietin A; scatter factor)]; HGS [hepatocyte growth factor-regulated tyrosine kinase substrate]; HGSNAT [heparan-alpha-glucosaminide N-acetyltransferase]; HIF1A [hypoxia inducible factor 1, alpha subunit (basic helix-loop-helix transcription factor)]; HINFP [histone H4 transcription factor]; HINT1 [histidine triad nucleotide binding protein 1]; HIPK2 [homeodomain interacting protein kinase 2]; HIRA [HIR histone cell cycle regulation defective homolog A (S. cerevisiae)]; HIST1H1B [histone cluster 1, H1b]; HIST1H3E [histone cluster 1, H3e]; HIST2H2AC [histone cluster 2, H2ac]; HIST2H3C [histone cluster 2, H3c]; HIST4H4 [histone cluster 4, H4]; HJURP [Holliday junction recognition protein]; HK2 [hexokinase 2]; HLA-A [major histocompatibility complex, class I, A]; HLA-B [major histocompatibility complex, class I, B]; HLA-C [major histocompatibility complex, class I, C]; HLA-DMA [major histocompatibility complex, class II, OM alpha]; HLA-DMB [major histocompatibility complex, class II, DM beta]; HLA-DOA [major histocompatibility complex, class II, DO alpha]; HLA-DOB [major histocompatibility complex, class II, DO beta]; HLA-DPA1 [major histocompatibility complex, class II, DP alpha 1]; HLA-DPB1 [major histocompatibility complex, class II, DP beta 1]; HLA-DQA1 [major histocompatibility complex, class II, DQ alpha 1]; HLA-DQA2 [major histocompatibility complex, class II, DQ alpha 2]; HLA-DQB1 [major histocompatibility complex, class II, DQ beta 1]; HLA-DRA [major histocompatibility complex, class II, DR alpha]; HLA-DRB1 [major histocompatibility complex, class II, DR beta 1]; HLA-DRB3 [major histocompatibility complex, class II, DR beta 3]; HLA-DRB4 [major histocompatibility complex, class II, DR beta 4]; HLA-DRB5 [major histocompatibility complex, class II, DR beta 5]; HLA-E [major histocompatibility complex, class I, E]; HLA-F [major histocompatibility complex, class I, F]; HLA-G [major histocompatibility complex, class I, G]; HLCS [holocarboxylase synthetase (biotin-(proprionyl-Coenzyme A-carboxylase (ATP-hydrolysing)) ligase)]; HLTF [helicase-like transcription factor]; HLX [H2.0-like homeobox]; HMBS [hydroxymethylbilane synthase]; HMGA1 [high mobility group AT-hook 1]; HMGB1 [high-mobility group box 1]; HMGCR [3-hydroxy-3-methylglutaryl-Coenzyme A reductase]; HMOX1 [heme oxygenase (decycling) 1]; HMOX2 [heme oxygenase (decycling) 2]; HNF1A [HNF1 homeoboxA]; HNF4A [hepatocyte nuclear factor 4, alpha]; HNMT [histamine N-methyltransferase]; HNRNPA1 [heterogeneous nuclear ribonucleoprotein A1]; HNRNPA2B1 [heterogeneous nuclear ribonucleoprotein A2/B1]; HNRNPH2 [heterogeneous nuclear ribonucleoprotein H2 (H′)]; HNRNPUL1 [heterogeneous nuclear ribonucleoprotein U-like 1]; HOXA13 [homeobox A13]; HOXA4 [homeobox A4]; HOXA9 [homeobox A9]; HOXB4 [homeobox B4]; HP [haptoglobin]; HPGDS [hematopoietic prostaglandin D synthase]; HPR [haptoglobin-related protein]; HPRT1 [hypoxanthine phosphoribosyltransferase 1]; HPS1 [Hermansky-Pudlak syndrome 1]; HPS3 [Hermansky-Pudlak syndrome 3]; HPS4 [Hermansky-Pudlak syndrome 4]; HPSE [heparanase]; HPX [hemopexin]; HRAS [v-Ha-ras Harvey rat sarcoma viral oncogene homolog]; HRG [histidine-rich glycoprotein]; HRH1 [histamine receptor H1]; HRH2 [histamine receptor H2]; HRH3 [histamine receptor H3]; HRH4 [histamine receptor H4]; HSD11B1 [hydroxysteroid (11-beta) dehydrogenase 1]; HSD11B2 [hydroxysteroid (11-beta) dehydrogenase 2]; HSD17B1 [hydroxysteroid (17-beta) dehydrogenase 1]; HSD17B4 [hydroxysteroid (17-beta) dehydrogenase 4]; HSF1 [heat shock transcription factor 1]; HSP90AA1 [heat shock protein 90 kDa alpha (cytosolic), class A member 1]; HSP90AB1 [heat shock protein 90 kDa alpha (cytosolic), class B member 1]; HSP90B1 [heat shock protein 90 kDa beta (Grp94), member 1]; HSPA14 [heat shock 70 kDa protein 14]; HSPA1A [heat shock 70 kDa protein 1A]; HSPA1B [heat shock 70 kDa protein IB]; HSPA2 [heat shock 70 kDa protein 2]; HSPA4 [heat shock 70 kDa protein 4]; HSPA5 [heat shock 70 kDa protein 5 (glucose-regulated protein, 78 kDa)]; HSPA8 [heat shock 70 kDa protein 8]; HSPB1 [heat shock 27 kDa protein 1]; HSPB2 [heat shock 27 kDa protein 2]; HSPD1 [heat shock 60 kDa protein 1 (chaperonin)]; HSPE1 [heat shock 10 kDa protein 1 (chaperonin 10)]; HSPG2 [heparan sulfate proteoglycan 2]; HTN3 [histatin 3]; HTR1A [5-hydroxytryptamine (serotonin) receptor 1A]; HTR2A [5-hydroxytryptamine (serotonin) receptor 2A]; HTR3A [5-hydroxytryptamine (serotonin) receptor 3A]; HTRA1 [HtrA serine peptidase 1]; HTT [huntingtin]; HUS1 [HUS1 checkpoint homolog (S. pombe)]; HUWE1 [HECT, UBA and WWE domain containing 1]; HYAL1 [hyaluronoglucosaminidase 1]; HYLS1 [hydrolethalus syndrome 1]; IAPP [islet amyloid polypeptide]; IBSP [integrin-binding sialoprotein]; ICAM1 [intercellular adhesion molecule 1]; ICAM2 [intercellular adhesion molecule 2]; ICAM3 [intercellular adhesion molecule 3]; ICAM4 [intercellular adhesion molecule 4 (Landsteiner-Wiener blood group)]; ICOS [inducible T-cell co-stimulator]; ICOSLG [inducible T-cell co-stimulator ligand]; ID1 [inhibitor of DNA binding 1, dominant negative helix-loop-helix protein]; ID2 [inhibitor of DNA binding 2, dominant negative helix-loop-helix protein]; IDO1 [indoleamine 2 [3-dioxygenase 1]; IDS [iduronate 2-sulfatase]; IDUA [iduronidase, alpha-L-]; IF127 [interferon, alpha-inducible protein 27]; IFI30 [interferon, gamma-inducible protein 30]; IFITM1 [interferon induced transmembrane protein 1 (9-27)]; IFNA 1 [interferon, alpha 1]; IFNA 2 [interferon, alpha 2]; IFNAR1 [interferon (alpha, beta and omega) receptor 1]; IFNAR2 [interferon (alpha, beta and omega) receptor 2]; IFNB1 [interferon, beta 1, fibroblast]; IFNG [interferon, gamma]; IFNGR1 [interferon gamma receptor 1]; IFNGR2 [interferon gamma receptor 2 (interferon gamma transducer 1)]; IGF1 [insulin-like growth factor 1 (somatomedin C)]; IGF1R [insulin-like growth factor 1 receptor]; IGF2 [insulin-like growth factor 2 (somatomedin A)]; IGF2R [insulin-like growth factor 2 receptor]; IGFBP1 [insulin-like growth factor binding protein 1]; IGFBP2 [insulin-like growth factor binding protein 2, 36 kDa]; IGFBP3 [insulin-like growth factor binding protein 3]; IGFBP4 [insulin-like growth factor binding protein 4]; IGFBP5 [insulin-like growth factor binding protein 5]; IGHA1 [immunoglobulin heavy constant alpha 1]; IGHE [immunoglobulin heavy constant epsilon]; IGHG1 [immunoglobulin heavy constant gamma 1 (G1 m marker)]; IGHG3 [immunoglobulin heavy constant gamma 3 (G3m marker)]; IGHG4 [immunoglobulin heavy constant gamma 4 (G4m marker)]; IGHM [immunoglobulin heavy constant mu]; IGHMBP2 [immunoglobulin mu binding protein 2]; IGKC [immunoglobulin kappa constant]; IGKV2D-29 [immunoglobulin kappa variable 2D-29]; IGLL1 [immunoglobulin lambda-like polypeptide 1]; IGSF1 [immunoglobulin superfamily, member 1]; IKBKAP [inhibitor of kappa light polypeptide gene enhancer in B-cells, kinase complex-associated protein]; IKBKB [inhibitor of kappa light polypeptide gene enhancer in B-cells, kinase beta]; IKBKE [inhibitor of kappa light polypeptide gene enhancer in B-cells, kinase epsilon]; IKBKG [inhibitor of kappa light polypeptide gene enhancer in B-cells, kinase gamma]; IKZF1 [IKAROS family zinc finger 1 (Ikaros)]; IKZF2 [IKAROS family zinc finger 2 (Helios)]; IL10 [interleukin 10]; Ill0RA [interleukin 10 receptor, alpha]; IL10RB [interleukin 10 receptor, beta]; IL11 [interleukin 11]; IL12A [interleukin 12A (natural killer cell stimulatory factor 1, cytotoxic lymphocyte maturation factor 1, p35)]; IL12B [interleukin 12B (natural killer cell stimulatory factor 2, cytotoxic lymphocyte maturation factor 2, p40)]; IL12RB1 [interleukin 12 receptor, beta 1]; IL12RB2 [interleukin 12 receptor, beta 2]; IL13 [interleukin 13]; IL13RA1 [interleukin 13 receptor, alpha 1]; IL13RA2 [interleukin 13 receptor, alpha 2]; IL15 [interleukin 15]; IL15RA [interleukin 15 receptor, alpha]; IL16 [interleukin 16 (lymphocyte chemoattractant factor)]; IL17A [interleukin 17A]; IL17F [interleukin 17F]; IL17RA [interleukin 17 receptor A]; IL17RB [interleukin 17 receptor B]; IL17RC [interleukin 17 receptor C]; IL18 [interleukin 18 (interferon-gamma-inducing factor)]; IL18BP [interleukin 18 binding protein]; IL18R1 [interleukin 18 receptor 1]; IL18RAP [interleukin 18 receptor accessory protein]; IL19 [interleukin 19]; ILIA [interleukin 1, alpha]; IL1B [interleukin 1, beta]; IL1F9 [interleukin 1 family, member 9]; IL1R1 [interleukin 1 receptor, type I]; IL1RAP [interleukin 1 receptor accessory protein]; IL1RL1 [interleukin 1 receptor-like 1]; IL1RN [interleukin 1 receptor antagonist]; IL2 [interleukin 2]; IL20 [interleukin 20]; IL21 [interleukin 21]; IL21R [interleukin 21 receptor]; IL22 [interleukin 22]; IL23A [interleukin 23, alpha subunit p19]; IL23R [interleukin 23 receptor]; IL24 [interleukin 24]; IL25 [interleukin 25]; IL26 [interleukin 26]; IL27 [interleukin 27]; IL27RA [interleukin 27 receptor, alpha]; IL29 [interleukin 29 (interferon, lambda 1)]; IL2RA [interleukin 2 receptor, alpha]; IL2RB [interleukin 2 receptor, beta]; IL2RG [interleukin 2 receptor, gamma (severe combined immunodeficiency)]; IL3 [interleukin 3 (colony-stimulating factor, multiple)]; IL31 [interleukin 31]; IL32 [interleukin 32]; IL33 [interleukin 33]; IL3RA [interleukin 3 receptor, alpha (low affinity)]; IL4 [interleukin 4]; IL4R [interleukin 4 receptor]; IL5 [interleukin 5 (colony-stimulating factor, eosinophil)]; IL5RA [interleukin 5 receptor, alpha]; IL6 [interleukin 6 (interferon, beta 2)]; IL6R [interleukin 6 receptor]; IL6ST [interleukin 6 signal transducer (gp130, oncostatin M receptor)]; IL7 [interleukin 7]; IL7R [interleukin 7 receptor]; IL8 [interleukin 8]; IL9 [interleukin 9]; IL9R [interleukin 9 receptor]; ILK [integrin-linked kinase]; IMPS [intramembrane protease 5]; INCENP [inner centromere protein antigens 135/155 kDa]; 1NG1 [inhibitor of growth family, member 1]; INHA [inhibin, alpha]; INHBA [inhibin, beta A]; INPP4A [inositol polyphosphate-4-phosphatase, type I, 107 kDa]; INPP5D [inositol polyphosphate-5-phosphatase, 145 kDa]; INPP5E [inositol polyphosphate-5-phosphatase, 72 kDa]; INPPL1 [inositol polyphosphate phosphatase-like 1]; INS [insulin]; INSL3 [insulin-like 3 (Leydig cell)]; INSR [insulin receptor]; IP013 [importin13]; IP07 [importin 7]; IQGAP1 [IQ motif containing GTPase activating protein 1]; IRAK1 [interleukin-1 receptor-associated kinase 1]; IRAK3 [interleukin-1 receptor-associated kinase 3]; IRAK4 [interleukin-1 receptor-associated kinase 4]; IRF1 [interferon regulatory factor 1]; IRF2 [interferon regulatory factor 2]; IRF3 [interferon regulatory factor 3]; IRF4 [interferon regulatory factor 4]; IRF5 [interferon regulatory factor 5]; IRF7 [interferon regulatory factor 7]; IRF8 [interferon regulatory factor 8]; IRGM [immunity-related GTPase family, M]; IRS 1 [insulin receptor substrate 1]; IRS2 [insulin receptor substrate 2]; IRS4 [insulin receptor substrate 4]; ISG15 [ISG15 ubiquitin-like modifier]; ITCH [itchy E3 ubiquitin protein ligase homolog (mouse)]; ITFG1 [integrin alpha FG-GAP repeat containing 1]; ITGA1 [integrin, alpha 1]; ITGA2 [integrin, alpha 2 (CD49B, alpha 2 subunit of VLA-2 receptor)]; ITGA2B [integrin, alpha 2b (platelet glycoprotein IIb of IIb/IIIa complex, antigen CD41)]; ITGA3 [integrin, alpha 3 (antigen CD49C, alpha 3 subunit of VLA-3 receptor)]; ITGA4 [integrin, alpha 4 (antigen CD49D, alpha 4 subunit of VLA-4 receptor)]; ITGA5 [integrin, alpha 5 (fibronectin receptor, alpha polypeptide)]; ITGA6 [integrin, alpha 6]; ITGA8 [integrin, alpha 8]; ITGAE [integrin, alpha E (antigen CD103, human mucosal lymphocyte antigen 1; alpha polypeptide)]; ITGAL [integrin, alpha L (antigen CD11A (p180), lymphocyte function-associated antigen 1; alpha polypeptide)]; ITGAM [integrin, alpha M (complement component 3 receptor 3 subunit)]; ITGAV [integrin, alpha V (vitronectin receptor, alpha polypeptide, antigen CD51)]; ITGAX [integrin, alpha X (complement component 3 receptor 4 subunit)]; ITGB1 [integrin, beta 1 (fibronectin receptor, beta polypeptide, antigen CD29 includes MDF2, MSK12)]; ITGB2 [integrin, beta 2 (complement component 3 receptor 3 and 4 subunit)]; ITGB3 [integrin, beta 3 (platelet glycoprotein IIIa, antigen CD61)]; ITGB3BP [integrin beta 3 binding protein (beta3-endonexin)]; ITGB4 [integrin, beta 4]; ITGB6 [integrin, beta 6]; ITGB7 [integrin, beta 7]; ITIH4 [inter-alpha (globulin) inhibitor H4 (plasma Kallikrein-sensitive glycoprotein)]; ITK [IL2-inducible T-cell kinase]; ITLN1 [intelectin 1 (galactofuranose binding)]; ITLN2 [intelectin 2]; ITPA [inosine triphosphatase (nucleoside triphosphate pyrophosphatase)]; ITPR1 [inositol1,4,5-triphosphate receptor, type 1]; ITPR3 [inositol1,4,5-triphosphate receptor, type 3]; IVD [isovaleryl Coenzyme A dehydrogenase]; IVL [involucrin]; IVNS1ABP [influenza virus NS1A binding protein]; JAG1 [jagged 1 (Alagille syndrome)]; JAK1 [Janus kinase 1]; JAK2 [Janus kinase 2]; JAK3 [Janus kinase 3]; JAKMIP1 [janus kinase and microtubule interacting protein1]; JMJD6 [jumonji domain containing 6]; JPH4 [junctophilin 4]; JRKL [jerky homolog-like (mouse)]; JUN [jun oncogene]; JUND [jun D proto-oncogene]; JUP [junction plakoglobin]; KARS [lysyl-tRNA synthetase]; KAT5 [K(lysine) acetyltransferase 5]; KCNA2 [potassium voltage-gated channel, shaker-related subfamily, member 2]; KCNA5 [potassium voltage-gated channel, shaker-related subfamily, member 5]; KCND1 [potassium voltage-gated channel, Shal-related subfamily, member 1]; KCNH2 [potassium voltage-gated channel, subfamily H (eag-related), member 2]; KCNIP4 [Kv channel interacting protein 4]; KCNMA1 [potassium large conductance calcium-activated channel, subfamily M, alpha member 1]; KCNMB1 [potassium large conductance calcium-activated channel, subfamily M, beta member 1]; KCNN3 [potassium intermediate/small conductance calcium-activated channel, subfamily N, member 3]; KCNS3 [potassium voltage-gated channel, delayed-rectifier, subfamily S, member 3]; KDR [kinase insert domain receptor (a type III receptor tyrosine kinase)]; KHDRBS1 [KH domain containing, RNA binding, signal transduction associated 1]; KHDRBS3 [KH domain containing, RNA binding, signal transduction associated 3]; KIAA0101 [KIAA0101]; KIF16B [kinesin family member 16B]; KIF20B [kinesin family member 20B]; KIF21B [kinesin family member 21B]; KIF22 [kinesin family member 22]; KIF2B [kinesin family member 2B]; KTF2C [kinesin family member 20]; KTR2DL1 [killer cell immunoglobulin-like receptor, two domains, long cytoplasmic tail, 1]; KIR2DL2 [killer cell immunoglobulin-like receptor, two domains, long cytoplasmic tail, 2]; KIR2DL3 [killer cell immunoglobulin-like receptor, two domains, long cytoplasmic tail, 3]; KIR2DL5A [killer cell immunoglobulin-like receptor, two domains, long cytoplasmic tail, 5A]; KIR2DS1 [killer cell immunoglobulin-like receptor, two domains, short cytoplasmic tail, 1]; KIR2DS2 [killer cell immunoglobulin-like receptor, two domains, shmi cytoplasmic tail, 2]; KIR2DS5 [killer cell immunoglobulin-like receptor, two domains, shmi cytoplasmic tail, 5]; KIR3DL1 [killer cell immunoglobulin-like receptor, three domains, long cytoplasmic tail, 1]; KIR3DS1 [killer cell immunoglobulin-like receptor, three domains, short cytoplasmic tail, 1]; KISS1 [KiSS-1 metastasis-suppressor]; KISSIR [KISS1 receptor]; KIT [v-kit Hardy-Zuckerman 4 feline sarcoma viral oncogene homolog]; KITLG [KIT ligand]; KLF2 [Kruppel-like factor 2 (lung)]; KLF4 [Kruppel-like factor 4 (gut)]; KLK1 [kallikrein 1]; KLK11 [kallikrein-related peptidase 11]; KLK3 [kallikrein-related peptidase 3]; KLKB1 [kallikrein B, plasma (Fletcher factor) 1]; KLRB1 [killer cell lectin-like receptor subfamily B, member 1]; KLRC1 [killer cell lectin-like receptor subfamily C, member 1]; KLRD1 [killer cell lectin-like receptor subfamily D, member 1]; KLRK1 [killer cell lectin-like receptor subfamily K, member 1]; KNG1 [kininogen 1]; KPNA1 [karyopherin alpha 1 (importin alpha 5)]; KPNA2 [karyopherin alpha 2 (RAG cohort 1, importin alpha 1)]; KPNB1 [karyopherin (importin) beta 1]; KRAS [v-Ki-ras2 Kirsten rat sarcoma viral oncogene homolog]; KRT1 [keratin 1]; KRT10 [keratin 10]; KRT13 [keratin 13]; KRT14 [keratin 14]; KRT16 [keratin 16]; KRT18 [keratin 18]; KRT19 [keratin 19]; KRT20 [keratin 20]; KRT5 [keratin 5]; KRT7 [keratin 7]; KRT8 [keratin 8]; KRT9 [keratin 9]; KRTAP19-3 [keratin associated protein 19-3]; KRTAP2-1, keratin associated protein 2-1]; L1CAM [L1 cell adhesion molecule]; LACTB [lactamase, beta]; LAG3 [lymphocyte-activation gene 3]; LALBA [lactalbumin, alpha-]; LAMA1 [laminin, alpha 1]; LAMA2 [laminin, alpha 2]; LAMA3 [laminin, alpha 3]; LAMA4 [laminin, alpha4]; LAMB1 [laminin, beta 1]; LAMB2 [laminin, beta 2 (laminin S)]; LAMB3 [laminin, beta 3]; LAMC1 [laminin, gamma 1 (formerly LAMB2)]; LAMC2 [laminin, gamma 2]; LAMP1 [lysosomal-associated membrane protein 1]; LAMP2 [lysosomal-associated membrane protein 2]; LAMP3 [lysosomal-associated membrane protein 3]; LAP3 [leucine aminopeptidase 3]; LAPTM4A [lysosomal protein transmembrane 4 alpha]; LAT [linker for activation of T cells]; LBP [lipopolysaccharide binding protein]; LBR [lamin B receptor]; LBXCOR1 [Lbxcor 1 homolog (mouse)]; LCAT [lecithin-cholesterol acyltransferase]; LCK [lymphocyte-specific protein tyrosine kinase]; LCN1 [lipocalin 1 (tear prealbumin)]; LCN2 [lipocalin 2]; LCP1 [lymphocyte cytosolic protein 1 (L-plastin)]; LCT [lactase]; LDLR [low density lipoprotein receptor]; LDLRAP1 [low density lipoprotein receptor adaptor protein 1]; LECT2 [leukocyte cell-derived chemotaxin 2]; LELP1 [late cornified envelope-like proline-rich 1]; LEMD3 [LEM domain containing 3]; LEP [leptin]; LEPR [leptin receptor]; LGALS1 [lectin, galactoside-binding, soluble, 1]; LGALS3 [lectin, galactoside-binding, soluble, 3]; LGALS3BP [lectin, galactoside-binding, soluble, 3 binding protein]; LGALS4 [lectin, galactoside-binding, soluble, 4]; LGALS9 [lectin, galactoside-binding, soluble, 9]; LGALS9B [lectin, galactoside-binding, soluble, 9B]; LGR4 [leucine-rich repeat-containing G protein-coupled receptor 4]; LHCGR [luteinizing hormone/choriogonadotropin receptor]; LIF [leukemia inhibitory factor (cholinergic differentiation factor)]; LIFR [leukemia inhibitory factor receptor alpha]; LIG1 [ligase I, DNA, ATP-dependent]; LIG3 [ligase III, DNA, ATP-dependent]; LIG4 [ligase IV, DNA, ATP-dependent]; LILRA3 [leukocyte immunoglobulin-like receptor, subfamily A (without TM domain), member 3]; LILRB4 [leukocyte immunoglobulin-like receptor, subfamily B (with TM and ITIM domains), member 4]; LIMS1 [LIM and senescent cell antigen-like domains 1]; LIPA [lipase A, lysosomal acid, cholesterol esterase]; LIPC [lipase, hepatic]; LIPE [lipase, hormone-sensitive]; LIPG [lipase, endothelial]; LMAN1 [lectin, mannose-binding, 1]; LMLN [lcishmanolysin-like (metallopeptidase M8 family)]; LMNA [lamin NC]; LMNB1 [lamin BI]; LMNB2 [lamin B2]; LOC646627 [phospholipase inhibitor]; LOX [lysyl oxidase]; LOXHD1 [lipoxygenase homology domains 1]; LOXL1 [lysyl oxidase-like 1]; LPA [lipoprotein, Lp(a)]; LPAR3 [lysophosphatidic acid receptor 3]; LPCAT2 [lysophosphatidylcholine acyltransferase 2]; LPL [lipoprotein lipase]; LPO [lactoperoxidase]; LPP [LIM domain containing preferred translocation partner in lipoma]; LRBA [LPS-responsive vesicle trafficking, beach and anchor containing]; LRP1 [low density lipoprotein receptor-related protein 1]; LRP6 [low density lipoprotein receptor-related protein 6]; LRPAP1 [low density lipoprotein receptor-related protein associated protein 1]; LRRC32 [leucine rich repeat containing 32]; LRRC37B [leucine rich repeat containing 37B]; LRRC8A [leucine rich repeat containing 8 family, member A]; LRRK2 [leucine-rich repeat kinase 2]; LRTOMT [leucine rich transmembrane and O-methyltransferase domain containing]; LSM1 [LSM1 homolog, U6 small nuclear RNA associated (S. cerevisiae)]; LSM2 [LSM2 homolog, U6 small nuclear RNA associated (S. cerevisiae)]; LSP1 [lymphocyte-specific protein 1]; LTA [lymphotoxin alpha (TNF superfamily, member 1)]; LTA4H [leukotriene A4 hydrolase]; LTB [lymphotoxin beta (TNF superfamily, member 3)]; LTB4R [leukotriene B4 receptor]; LTB4R2 [leukotriene B4 receptor 2]; LTBR [lymphotoxin beta receptor (TNFR superfamily, member 3)]; LTC4S [leukotriene C4 synthase]; LTF [lactotransferrin]; LY86 [lymphocyte antigen 86]; LY9 [lymphocyte antigen 9]; LYN [v-yes-1 Yamaguchi sarcoma viral related oncogene homolog]; LYRM4 [LYR motif containing 4]; LYST [lysosomal trafficking regulator]; LYZ [lysozyme (renal amyloidosis)]; LYZL6 [lysozyme-like 6]; LZTR1 [leucine-zipper-like transcription regulator 1]; M6PR [mannose-6-phosphate receptor (cation dependent)]; MADCAM1 [mucosal vascular addressin cell adhesion molecule 1]; MAF [v-mafmusculoaponeurotic fibrosarcoma oncogene homolog (avian)]; MAG [myelin associated glycoprotein]; MAN2A1 [mannosidase, alpha, class 2A, member 1]; MAN2B1 [mannosidase, alpha, class 2B, member 1]; MANBA [mannosidase, beta A, lysosomal]; MANF [mesencephalic astrocyte-derived neurotrophic factor]; MAOB [monoamine oxidase B]; MAP2 [microtubule-associated protein 2]; MAP2K1 [mitogen-activated protein kinase kinase 1]; MAP2K2 [mitogen-activated protein kinase kinase 2]; MAP2K3 [mitogen-activated protein kinase kinase 3]; MAP2K4 [mitogen-activated protein kinase kinase 4]; MAP3K1 [mitogen-activated protein kinase kinase kinase 1]; MAP3K11 [mitogen-activated protein kinase kinase kinase 11]; MAP3K14 [mitogen-activated protein kinase kinase kinase 14]; MAP3K5 [mitogen-activated protein kinase kinase kinase 5]; MAP3K7 [mitogen-activated protein kinase kinase kinase 7]; MAP3K9 [mitogen-activated protein kinase kinase kinase 9]; MAPK1 [mitogen-activated protein kinase 1]; MAPK10 [mitogen-activated protein kinase 10]; MAPK11 [mitogen-activated protein kinase 11]; MAPK12 [mitogen-activated protein kinase 12]; MAPK13 [mitogen-activated protein kinase 13]; MAPK14 [mitogen-activated protein kinase 14]; MAPK3 [mitogen-activated protein kinase 3]; MAPK8 [mitogen-activated protein kinase 8]; MAPK9 [mitogen-activated protein kinase 9]; MAPKAP1 [mitogen-activated protein kinase associated protein 1]; MAPKAPK2 [mitogen-activated protein kinase-activated protein kinase 2]; MAPKAPK5 [mitogen-activated protein kinase-activated protein kinase 5]; MAPT [microtubule-associated protein tau]; MARCKS [myristoylated alanine-rich protein kinase C substrate]; MASP2 [mannan-binding lectin serine peptidase 2]; MATN1 [matrilin 1, cartilage matrix protein]; MAVS [mitochondrial antiviral signaling protein]; MB [myoglobin]; MBD2 [methyl-CpG binding domain protein 2]; MBL2 [mannose-binding lectin (protein C) 2, soluble (opsonic defect)]; MBP [myelin basic protein]; MBTPS2 [membrane-bound transcription factor peptidase, site 2]; MC2R [melanocortin 2 receptor (adrenocorticotropic hormone)]; MC3R [melanocortin 3 receptor]; MC4R [melanocortin 4 receptor]; MCCC2 [methylcrotonoyl-Coenzyme A carboxylase 2 (beta)]; MCHR1 [melanin-concentrating hormone receptor 1]; MCL1 [myeloid cell leukemia sequence 1 (BCL2-related)]; MCM2 [minichromosome maintenance complex component 2]; MCM4 [minichromosome maintenance complex component 4]; MCOLN1 [mucolipin 1]; MCPH1 [microcephalin 1]; MDC1 [mediator of DNA-damage checkpoint 1]; MDH2 [malate dehydrogenase 2, NAD (mitochondrial)]; MDM2 [Mdm2 p53 binding protein homolog (mouse)]; ME2 [malic enzyme 2, NAD(+)-dependent, mitochondrial]; MECOM [MDS1 and EVI1 complex locus]; MED1 [mediator complex subunit 1]; MED12 [mediator complex subunit 12]; MED15 [mediator complex subunit 15]; MED28 [mediator complex subunit 28]; MEFV [Mediterranean fever]; MEN1 [multiple endocrine neoplasia I]; MEPE [matrix extracellular phosphoglycoprotein]; MERTK [c-mer proto-oncogene tyrosine kinase]; MESP2 [mesoderm posterior 2 homolog (mouse)]; MET [met proto-oncogene (hepatocyte growth factor receptor)]; MGAM [maltase-glucoamylase (alpha-glucosidase)]; MGAT1 [mannosyl (alpha-1,3-)-glycoprotein beta-1,2-N-acetylglucosaminyltransferase]; MGAT2 [mannosyl (alpha-1,6-)-glycoprotein beta-1,2-N-acetylglucosaminyltransferase]; MGLL [monoglyceride lipase]; MGMT [0-6-methylguanine-DNA methyltransferase]; MGST2 [microsomal glutathione S-transferase 2]; MICA [MHC class I polypeptide-related sequence A]; MICB [MHC class I polypeptide-related sequence B]; MIF [macrophage migration inhibitory factor (glycosylation-inhibiting factor)]; MK167 [antigen identified by monoclonal antibody Ki-67]; MKS1 [Meckel syndrome, type 1]; MLH1 [mutL homolog 1, colon cancer, nonpolyposis type 2 (E. coli)]; MLL [myeloid/lymphoid or mixed-lineage leukemia (trithorax homolog, Drosophila)]; MLLT4 [myeloid/lymphoid or mixed-lineage leukemia (trithorax homolog, Drosophila); translocated to, 4]; MLN [motilin]; MLXTPL [MLX interacting protein-like]; MMAA [methylmalonic aciduria (cobalamin deficiency) cb1A type]; MMAB [methylmalonic aciduria (cobalamin deficiency) cb1B type]; MMACHC [methylmalonic aciduria (cobalamin deficiency) cb1C type, with homocystinuria]; MME [membrane metallo-endopeptidase]; MMP1 [matrix metallopeptidase 1 (interstitial collagenase)]; MMP10 [matrix metallopeptidase 10 (stromelysin 2)]; MMP12 [matrix metallopeptidase 12 (macrophage elastase)]; MMP13 [matrix metallopeptidase 13 (collagenase 3)]; MMP14 [matrix metallopeptidase 14 (membrane-inserted)]; MMP15 [matrix metallopeptidase 15 (membrane-inserted)]; MMP17 [matrix metallopeptidase 17 (membrane-inserted)]; MMP2 [matrix metallopeptidase 2 (gelatinase A, 72 kDa gelatinase, 72 kDa type IV collagenase)]; MMP20 [matrix metallopeptidase 20]; MMP21 [matrix metallopeptidase 21]; MMP28 [matrix metallopeptidase 28]; MMP3 [matrix metallopeptidase 3 (stromelysin 1, progelatinase)]; MMP7 [matrix metallopeptidase 7 (matrilysin, uterine)]; MMPR [matrix metallopeptidase R (neutrophil collagenase)]; MMP9 [matrix metallopeptidase 9 (gelatinase B, 92 kDa gelatinase, 92 kDa type IV collagenase)]; MMRN1 [multimerin 1]; MNAT1 [menage a trois homolog 1, cyclin H assembly factor (Xenopus laevis)]; MOG [myelin oligodendrocyte glycoprotein]; MOGS [mannosyl-oligosaccharide glucosidase]; MPG [N-methylpurine-DNA glycosylase]; MPL [myeloproliferative leukemia virus oncogene]; MPO [myeloperoxidase]; MPZ [myelin protein zero]; MR1 [major histocompatibility complex, class !-related]; MRC1 [mannose receptor, C type 1]; MRC2 [mannose receptor, C type 2]; MRE11A [MRE11 meiotic recombination 11 homolog A (S. cerevisiae)]; MRGPRX1 [MAS-related GPR, member XI]; MRPL28 [mitochondrial ribosomal protein L28]; MRPL40 [mitochondrial ribosomal protein L40]; MRPS16 [mitochondrial ribosomal protein S16]; MRPS22 [mitochondrial ribosomal protein S22]; MS4A1 [membrane-spanning 4-domains, subfamily A, member 1]; MS4A2 [membrane-spanning 4-domains, subfamily A, member 2 (Fe fragment ofigE, high affinity I, receptor for; beta polypeptide)]; MS4A3 [membrane-spanning 4-domains, subfamily A, member 3 (hematopoietic cell-specific)]; MSH2 [mutS homolog 2, colon cancer, nonpolyposis type 1 (E. coli)]; MSH5 [mutS homolog 5 (E. coli)]; MSH6 [mutS homolog 6 (E. coli)]; MSLN [mesothelin]; MSN [moesin]; MSR1 [macrophage scavengerreceptor 1]; MST1 [macrophage stimulating 1 (hepatocyte growth factor-like)]; MST1R [macrophage stimulating 1 receptor (c-ruet-related tyrosine kinase)]; MSTN [myostatin]; MSX2 [msh homeobox 2]; MT2A [metallothionein 2A]; MTCH2 [mitochondrial carrier homolog 2 (C. elegans)]; MT-C02 [mitochondrially encoded cytochrome c oxidase II]; MTCP1 [mature T-cell proliferation 1]; MT-CYB [mitochondrially encoded cytochrome b]; MTHFD1 [methylenetetrahydrofolate dehydrogenase (NADP+dependent) 1, methenyltetrahydrofolate cyclohydrolase, formyltetrahydrofolate synthetase]; MTHFR [5 [10-methylenetetrahydrofolate reductase (NADPH)]; MTMR14 [myotubularin related protein 14]; MTMR2 [myotubularin related protein 2]; MT-ND1 [mitochondrially encoded NADH dehydrogenase 1]; MT-ND2 [mitochondrially encoded NADH dehydrogenase 2]; MTOR [mechanistic target of rapamycin (serine/threonine kinase)]; MTR [5-methyltetrahydrofolate-homocysteine methyltransferase]; MTRR [5-methyltetrahydrofolate-homocysteine methyltransferase reductase]; MTTP [microsomal triglyceride transfer protein]; MTX1 [metaxin 1]; MUC1 [mucin 1, cell surface associated]; MUC12 [mucin 12, cell surface associated]; MUC16 [mucin 16, cell surface associated]; MUC19 [mucin 19, oligomeric]; MUC2 [mucin 2, oligomeric mucus/gel-forming]; MUC3A [mucin 3A, cell surface associated]; MUC3B [mucin 3B, cell surface associated]; MUC4 [mucin 4, cell surface associated]; MUC5AC [mucin SAC, oligomeric mucus/gel-forming]; MUC5B [mucin 5B, oligomeric mucus/gel-forming]; MUC6 [mucin 6, oligomeric mucus/gel-forming]; MUC7 [mucin 7, secreted]; MUS81 [MUS81 endonuclease homolog (S. cerevisiae)]; MUSK [muscle, skeletal, receptor tyrosine kinase]; MUT [methylmalonyl Coenzyme A mutase]; MVK [mevalonate kinase]; MVP [major vault protein]; MX1 [myxovirus (influenza virus) resistance 1, interferon-inducible protein p78 (mouse)]; MYB [v-myb myeloblastosis viral oncogene homolog (avian)]; MYBPH [myosin binding protein H]; MYC [v-myc myelocytomatosis viral oncogene homolog (avian)]; MYCN [v-myc myelocytomatosis viral related oncogene, neuroblastoma derived (avian)]; MYD88 [myeloid differentiation primary response gene (88)]; MYH1 [myosin, heavy chain 1, skeletal muscle, adult]; MYH10 [myosin, heavy chain 10, non-muscle]; MYH11 [myosin, heavy chain 11, smooth muscle]; MYH14 [myosin, heavy chain 14, non-muscle]; MYH2 [myosin, heavy chain 2, skeletal muscle, adult]; MYH3 [myosin, heavy chain 3, skeletal muscle, embryonic]; MYH6 [myosin, heavy chain 6, cardiac muscle, alpha]; MYH7 [myosin, heavy chain 7, cardiac muscle, beta]; MYH8 [myosin, heavy chain 8, skeletal muscle, perinatal]; MYH9 [myosin, heavy chain 9, non-muscle]; MYL2 [myosin, light chain 2, regulatory, cardiac, slow]; MYL3 [myosin, light chain 3, alkali; ventricular, skeletal, slow]; MYL7 [myosin, light chain 7, regulatory]; MYL9 [myosin, light chain 9, regulatory]; MYLK [myosin light chain kinase]; MYO15A [myosin XVA]; MYO1A [myosin IA]; MY01F [myosin IF]; MY03A [myosin liIA]; MY05A [myosin VA (heavy chain 12, myoxin)]; MY06 [myosin VI]; MY07A [myosin VI1A]; MY09B [myosin IXB]; MYOC [myocilin, trabecular meshwork inducible glucocorticoid response]; MYOD1 [myogenic differentiation 1]; MYOM2 [myomesin (M-protein) 2, 165 kDa]; MYST1 [MYST histone acetyltransferase 1]; MYST2 [MYST histone acetyltransferase 2]; MYST3 [MYST histone acetyltransferase (monocytic leukemia) 3]; MYST4 [MYST histone acetyltransferase (monocytic leukemia) 4]; NAGA [N-acetylgalactosaminidase, alpha-]; NAGLU [N-acetylglucosaminidase, alpha-]; NAMPT [nicotinamide phosphoribosyltransferase]; NANOG [Nanog homeobox]; NANOS1 [nanos homolog 1 (Drosophila)]; NAPA [N-ethylmaleimide-sensitive factor attachment protein, alpha]; NAT1 [N-acetyltransferase 1 (arylamine N-acetyltransferase)]; NAT2 [N-acetyltransferase 2 (arylamine N-acetyltransferase)]; NAT9 [N-acetyltransferase 9 (GCN5-related, putative)]; NBEA [neurobeachin]; NBN [nibrin]; NCAM1 [neural cell adhesion molecule 1]; NCF1 [neutrophil cytosolic factor 1]; NCF2 [neutrophil cytosolic factor 2]; NCF4 [neutrophil cytosolic factor 4, 40 kDa]; NCK1 [NCK adaptor protein 1]; NCL [nucleolin]; NCOA1 [nuclear receptor coactivator 1]; NCOA2 [nuclear receptor coactivator 2]; NCOR1 [nuclear receptor co-repressor 1]; NCR3 [natural cytotoxicity triggering receptor 3]; NDUFA13 [NADH dehydrogenase (ubiquinone) 1 alpha subcomplex, 13]; NDUFAB1 [NADH dehydrogenase (ubiquinone) 1, alpha/beta subcomplex, 1, 8 kDa]; NDUFAF2 [NADH dehydrogenase (ubiquinone) 1 alpha subcomplex, assembly factor 2]; NEDD4 [neural precursor cell expressed, developmentally down-regulated 4]; NEFL [neurofilament, light polypeptide]; NEFM [neurofilament, medium polypeptide]; NEGRI [neuronal growth regulator 1]; NEK6 [NIMA (never in mitosis gene a)-related kinase 6]; NELF [nasal embryonic LHRH factor]; NELL1 [NEL-like 1 (chicken)]; NES [nestin]; NEU1 [sialidase 1 (lysosomal sialidase)]; NEUROD1 [neurogenic differentiation 1]; NF1 [neurofibromin 1]; NF2 [neurofibromin 2 (merlin)]; NFAT5 [nuclear factor of activated T-cells 5, tonicity-responsive]; NFATC1 [nuclear factor of activated T-cells, cytoplasmic, calcineurin-dependent 1]; NFATC2 [nuclear factor of activated T-cells, cytoplasmic, calcineurin-dependent 2]; NFATC4 [nuclear factor of activated T-cells, cytoplasmic, calcineurin-dependent 4]; NFE2L2 [nuclear factor (erythroid-derived 2)-like 2]; NFKB1 [nuclear factor of kappa light polypeptide gene enhancer in B-cells 1]; NFKB2 [nuclear factor of kappa light polypeptide gene enhancer in B-cells 2 (p49/pi 00)]; NFKBIA [nuclear factor of kappa light polypeptide gene enhancer in B-cells inhibitor, alpha]; NFKBIB [nuclear factor of kappa light polypeptide gene enhancer in B-cells inhibitor, beta]; NFKBIL1 [nuclear factor of kappa light polypeptide gene enhancer in B-cells inhibitor-like 1]; NFU1 [NFU1 iron-sulfur cluster scaffold homolog (S. cerevisiae)]; NGF [nerve growth factor (beta polypeptide)]; NGFR [nerve growth factor receptor (TNFR superfamily, member 16)]; NHEJ1 [nonhomologous end-joining factor 1]; NID1 [nidogen 1]; NKAP [NFkB activating protein]; NKX2-1, NK2 homeobox 1]; NKX2-3 [NK2 transcription factor related, locus 3 (Drosophila)]; NLRP3 [NLR family, pyrin domain containing 3]; NMB [neuromedin B]; NME1 [non-metastatic cells 1, protein (NM23A) expressed in]; NME2 [non-metastatic cells 2, protein (NM23B) expressed in]; NMU [neuromedin U]; NNAT [neuronatin]; NOD1 [nucleotide-binding oligomerization domain containing 1]; NOD2 [nucleotide-binding oligomerization domain containing 2]; NONO [non-POU domain containing, octamer-binding]; NOS 1 [nitric oxide synthase 1 (neuronal)]; NOS2 [nitric oxide synthase 2, inducible]; NOS3 [nitric oxide synthase 3 (endothelial cell)]; NOTCH1 [Notch homolog 1, translocation-associated (Drosophila)]; NOTCH2 [Notch homolog 2 (Drosophila)]; NOTCH3 [Notch homolog 3 (Drosophila)]; NOTCH4 [Notch homolog 4 (Drosophila)]; NOX1 [NADPH oxidase 1]; NOX3 [NADPH oxidase 3]; NOX4 [NADPH oxidase 4]; NOX5 [NADPH oxidase, EF-hand calcium binding domain 5]; NPAT [nuclear protein, ataxia-telangiectasia locus]; NPC 1 [Niemann-Pick disease, type C1]; NPC1L1 [NPC1 (Niemann-Pick disease, type C1, gene)-like 1]; NPC2 [Niemann-Pick disease, type C2]; NPHP1 [nephronophthisis 1 Guvenile)]; NPHS1 [nephrosis 1, congenital, Finnish type (nephrin)]; NPHS2 [nephrosis 2, idiopathic, steroid-resistant (podocin)]; NPLOC4 [nuclear protein localization 4 homolog (S. cerevisiae)]; NPM1 [nucleophosmin (nucleolar phosphoprotein B23, numatrin)]; NPPA [natriuretic peptide precursor A]; NPPB [natriuretic peptide precursor B]; NPPC [natriuretic peptide precursor C]; NPR1 [natriuretic peptide receptor A/guanylate cyclase A (atrionatriuretic peptide receptor A)]; NPR3 [natriuretic peptide receptor C/guanylate cyclase C (atrionatriuretic peptide receptor C)]; NPS [neuropeptide S]; NPSR1 [neuropeptide S receptor 1]; NPY [neuropeptide Y]; NPY2R [neuropeptide Y receptor Y2]; NQ01 [NAD(P)H dehydrogenase, quinone 1]; NROB1 [nuclear receptor subfamily 0, group B, member 1]; NR1H2 [nuclear receptor subfamily 1, group H, member 2]; NR1H3 [nuclear receptor subfamily 1, group H, member 3]; NR1H4 [nuclear receptor subfamily 1, group H, member 4]; NR112 [nuclear receptor subfamily 1, group 1, member 2]; NR 1 T3 [nuclear receptor subfamily 1, group T, member 3]; NR2F2 [nuclear receptor subfamily 2, group F, member 2]; NR3C1 [nuclear receptor subfamily 3, group C, member 1 (glucocorticoid receptor)]; NR3C2 [nuclear receptor subfamily 3, group C, member 2]; NR4A1 [nuclear receptor subfamily 4, group A, member 1]; NR4A3 [nuclear receptor subfamily 4, group A, member 3]; NR5A1 [nuclear receptor subfamily 5, group A, member 1]; NRF1 [nuclear respiratory factor 1]; NRG1 [neuregulin 1]; NRIP1 [nuclear receptor interacting protein 1]; NRTP2 [nuclear receptor interacting protein 2]; NRP1 [neuropilin 1]; NSD1 [nuclear receptor binding SET domain protein 1]; NSDHL [NAD(P) dependent steroid dehydrogenase-like]; NSF [N-ethylmaleimide-sensitive factor]; NT5E [5′-nucleotidase, ecto (CD73)]; NTAN1 [N-terminal asparagine amidase]; NTF3 [neurotrophin 3]; NTF4 [neurotrophin 4]; NTN1 [netrin 1]; NTRK1 [neurotrophic tyrosine kinase, receptor, type 1]; NTRK2 [neurotrophic tyrosine kinase, receptor, type 2]; NTRK3 [neurotrophic tyrosine kinase, receptor, type 3]; NTS [neurotensin]; NUCB2 [nucleobindin 2]; NUDT1 [nudix (nucleoside diphosphate linked moiety X)-type motif 1]; NUDT2 [nudix (nucleoside diphosphate linked moiety X)-type motif2]; NUDT6 [nudix (nucleoside diphosphate linked moiety X)-type motif6]; NUFIP2 [nuclear fragile X mental retardation protein interacting protein 2]; NUP98 [nucleoporin 98 kDa]; NXF1 [nuclear RNA export factor 1]; OCA2 [oculocutaneous albinism II]; OCLN [occludin]; ODC1 [ornithine decarboxylase 1]; OFD1 [oral-facial-digital syndrome 1]; OGDH [oxoglutarate (alpha-ketoglutarate) dehydrogenase (lipoamide)]; OGG1 [8-oxoguanine DNA glycosylase]; OGT [O-linked N-acetylglucosamine (G1cNAc) transferase (UDP-N-acetylglucosamine:polypeptide-N-acetylglucosaminyl transferase)]; OLR1 [oxidized low density lipoprotein (lectin-like) receptor 1]; OMP [olfactory marker protein]; ONECUT2 [one cut homeobox 2]; OPN3 [opsin 3]; OPRK1 [opioid receptor, kappa 1]; OPRM1 [opioid receptor, mu 1]; OPTN [optineurin]; OR2B11 [olfactory receptor, family 2, subfamily B, member 11]; ORMDL3 [ORM1-like 3 (S. cerevisiae)]; OSBP [oxysterol binding protein]; OSGIN2 [oxidative stress induced gro ih inhibitor family member 2]; OSM [oncostatin M]; OTC [ornithine carbamoyltransferase]; OTOP2 [otopetrin 2]; OTOP3 [otopetrin 3]; OTUD1 [OTU domain containing 1]; OXA1L [oxidase (cytochrome c) assembly 1-like]; OXER1 [oxoeicosanoid (OXE) receptor 1]; OXT [oxytocin, prepropeptide]; OXTR [oxytocin receptor]; P2RX7 [purinergic receptor P2X, ligand-gated ion channel, 7]; P2RY1 [purinergic receptor P2Y, G-protein coupled, 1]; P2RY12 [purinergic receptor P2Y, G-protein coupled, 12]; P2RY14 [purinergic receptor P2Y, G-protein coupled, 14]; P2RY2 [purinergic receptor P2Y, G-protein coupled, 2]; P4HA2 [proly14-hydroxylase, alpha polypeptide II]; P4HB [proly14-hydroxylase, beta polypeptide]; P4HTM [proly14-hydroxylase, transmembrane (endoplasmic reticulum)]; PABPC1 [poly(A) binding protein, cytoplasmic 1]; PACSIN3 [protein kinase C and casein kinase substrate in neurons 3]; PAEP [progestagen-associated endometrial protein]; PAFAH1B1 [platelet-activating factor acetylhydrolase lb, regulatory subunit 1 (45 kDa)]; PAH [phenylalanine hydroxylase]; PAK1 [p21 protein (Cdc42/Rac)-activated kinase 1]; PAK2 [p21 protein (Cdc42/Rac)-activated kinase 2]; PA10 [p21 protein (Cdc42/Rac)-activated kinase 3]; PAM [peptidylglycine alpha-amidating monooxygenase]; PAPPA [pregnancy-associated plasma protein A, pappalysin 1]; PARG [poly (ADP-ribose) glycohydrolase]; PARK2 [Parkinson disease (autosomal recessive, juvenile) 2, parkin]; PARP1 [poly (ADP-ribose) polymerase 1]; PAWR [PRKC, apoptosis, WT1, regulator]; PAX2 [paired box 2]; PAX3 [paired box 3]; PAX5 [paired box 5]; PAX6 [paired box 6]; PAXIP1 [PAX interacting (with transcription-activation domain) protein 1]; PC [pyruvate carboxylase]; PCCA [propionyl Coenzyme A carboxylase, alpha polypeptide]; PCCB [propionyl Coenzyme A carboxylase, beta polypeptide]; PCDH1 [protocadherin 1]; PCK1 [phosphoenolpyruvate carboxykinase 1 (soluble)]; PCM1 [pericentriolar material 1]; PCNA [proliferating cell nuclear antigen]; PCNT [pericentrin]; PCSK1 [proprotein convertase subtilisin/kexin type 1]; PCSK6 [proprotein convertase subtilisin/kexin type 6]; PCSK7 [proprotein convertase subtilisin/kexin type 7]; PCYT1A [phosphate cytidylyltransferase 1, choline, alpha]; PCYT2 [phosphate cytidylyltransferase 2, ethanolamine]; PDCD1 [programmed cell death 1]; PDCD1LG2 [programmed cell death 1 ligand 2]; PDCD6 [programmed cell death 6]; PDE3B [phosphodiesterase 3B, cGMP-inhibited]; PDE4A [phosphodiesterase 4A, cAMP-specific (phosphodiesterase E2 dunce homolog, Drosophila)]; PDE4B [phosphodiesterase 4B, cAMP-specific (phosphodiesterase E4 dunce homolog, Drosophila)]; PDE4D [phosphodiesterase 4D, cAMP-specific (phosphodiesterase E3 dunce homolog, Drosophila)]; PDE7A [phosphodiesterase 7A]; PDGFA [platelet-derived growth factor alpha polypeptide]; PDGFB [platelet-derived growth factor beta polypeptide (simian sarcoma viral (v-sis) oncogene homolog)]; PDGFRA [platelet-derived growth factor receptor, alpha polypeptide]; PDGFRB [platelet-derived growth factor receptor, beta polypeptide]; PDIA2 [protein disulfide isomerase family A, member 2]; PDIA3 [protein disulfide isomerase family A, member 3]; PDK1 [pyruvate dehydrogenase kinase, isozyme 1]; PDLIM1 [PDZ and LIM domain 1]; PDLIM5 [PDZ and LIM domain 5]; PDLIM7 [PDZ and LIM domain 7 (enigma)]; PDP1 [pyruvate dehyrogenase phosphatase catalytic subunit 1]; PDX1 [pancreatic and duodenal homeobox 1]; PDXK [pyridoxal (pyridoxine, vitamin B6) kinase]; PDYN [prodynorphin]; PECAM1 [platelet/endothelial cell adhesion molecule]; PEMT [phosphatidylethanolamine N-methyltransferase]; PENK [proenkephalin]; PEPD [peptidase D]; PERI [period homolog 1 (Drosophila)]; PEX1 [peroxisomal biogenesis factor 1]; PEX10 [peroxisomal biogenesis factor 10]; PEX12 [peroxisomal biogenesis factor 12]; PEX13 [peroxisomal biogenesis factor 13]; PEX14 [peroxisomal biogenesis factor 14]; PEX16 [peroxisomal biogenesis factor 16]; PEX19 [peroxisomal biogenesis factor 19]; PEX2 [peroxisomal biogenesis factor 2]; PEX26 [peroxisomal biogenesis factor 26]; PEX3 [peroxisomal biogenesis factor 3]; PEX5 [peroxisomal biogenesis factor 5]; PEX6 [peroxisomal biogenesis factor 6]; PEX7 [peroxisomal biogenesis factor 7]; PF4 [platelet factor 4]; PFAS [phosphoribosylfonnylglycinamidine synthase]; PFDN4 [prefoldin subunit 4]; PFN1 [profilin 1]; PGC [progastricsin (pepsinogen C)]; PGD [phosphogluconate dehydrogenase]; PGF [placental growth factor]; PGK1 [phosphoglycerate kinase 1]; PGM1 [phosphoglucomutase 1]; PGR [progesterone receptor]; PHB [prohibitin]; PHEX [phosphate regulating endopeptidase homolog, X-linked]; PHF11 [PHD finger protein 11]; PHOX2B [paired-like homeobox 2b]; PHTF1 [putative homeodomain transcription factor 1]; PHYH [phytanoyl-CoA 2-hydroxylase]; PHYHIP [phytanoyl-CoA 2-hydroxylase interacting protein]; PI3 [peptidase inhibitor 3, skin-derived]; PIGA [phosphatidylinositol glycan anchor biosynthesis, class A]; PIGR [polymeric immunoglobulin receptor]; P1K3C2A [phosphoinositide-3-kinase, class 2, alpha polypeptide]; P1K3C2B [phosphoinositide-3-kinase, class 2, beta polypeptide]; PTK3C2G [phosphoinositide-3-kinase, class 2, gamma polypeptide]; PIK3C3 [phosphoinositide-3-kinase, class 3]; PIK3CA [phosphoinositide-3-kinase, catalytic, alpha polypeptide]; PIK3CB [phosphoinositide-3-kinase, catalytic, beta polypeptide]; PIK3CD [phosphoinositide-3-kinase, catalytic, delta polypeptide]; PIK3CG [phosphoinositide-3-kinase, catalytic, gamma polypeptide]; PIK3R1 [phosphoinositide-3-kinase, regulatory subunit 1 (alpha)]; PIK3R2 [phosphoinositide-3-kinase, regulatory subunit 2 (beta)]; PTK3R3 [phosphoinositide-3-kinase, regulatory subunit 3 (gamma)]; PIKFYVE [phosphoinositide kinase, FYVE finger containing]; PIN1 [peptidylprolyl cis/trans isomerase, NIMA-interacting 1]; PINK1 [PTEN induced putative kinase 1]; PIP [prolactin-induced protein]; PIP5KL1 [phosphatidylinositol-4-phosphate 5-kinase-like 1]; PITPNM1 [phosphatidylinositol transfer protein, membrane-associated 1]; PITRM1 [pitrilysin metallopeptidase 1]; PITX2 [paired-like homeodomain 2]; PKD2 [polycystic kidney disease 2 (autosomal dominant)]; PKLR [pyruvate kinase, liver and RBC]; PKM2 [pyruvate kinase, muscle]; PKN1 [protein kinase N1]; PL-5283 [PL-5283 protein]; PLA2G1B [phospholipase A2, group IB (pancreas)]; PLA2G2A [phospholipase A2, group IIA (platelets, synovial fluid)]; PLA2G2D [phospholipase A2, group 1iD]; PLA2G4A [phospholipase A2, group IVA (cytosolic, calcium-dependent)]; PLA2G6 [phospholipase A2, group VI (cytosolic, calcium-independent)]; PLA2G7 [phospholipase A2, group VII (platelet-activating factor acetylhydrolase, plasma)]; PLA2R1 [phospholipase A2 receptor 1, 180 kDa]; PLAT [plasminogen activator, tissue]; PLAU [plasminogen activator, urokinase]; PLAUR [plasminogen activator, urokinase receptor]; PLCB1 [phospholipase C, beta 1 (phosphoinositide-specific)]; PLCB2 [phospholipase C, beta 2]; PLCB4 [phospholipase C, beta 4]; PLCD1 [phospholipase C, delta 1]; PLCG1 [phospholipase C, gamma 1]; PLCG2 [phospholipase C, gamma 2 (phosphatidylinositol-specific)]; PLD1 [phospholipase D1, phosphatidylcholine-specific]; PLEC [plectin]; PLEK [pleckstrin]; PLG [plasminogen]; PLIN1 [perilipin 1]; PLK1 [polo-like kinase 1 (Drosophila)]; PLK2 [polo-like kinase 2 (Drosophila)]; PLK3 [polo-like kinase 3 (Drosophila)]; PLP1 [proteolipid protein 1]; PLTP [phospholipid transfer protein]; PMAIP1 [phorbol-12-myristate-13-acetate-induced protein 1]; PMCH [pro-melanin-concentrating hormone]; PML [promyelocytic leukemia]; PMP22 [peripheral myelin protein 22]; PMS2 [PMS2 postmeiotic segregation increased 2 (S. cerevisiae)]; PNLIP [pancreatic lipase]; PNMA3 [paraneoplastic antigen MA3]; PNMT [phenylethanolamine N-methyltransferase]; PNP [purine nucleoside phosphorylase]; POLB [polymerase (DNA directed), beta]; POLD3 [polymerase (DNA-directed), delta 3, accessmy subunit]; POLD4 [polymerase (DNA-directed), delta 4]; POLH [polymerase (DNA directed), eta]; POLL [polymerase (DNA directed), lambda]; POLR2A [polymerase (RNA) II (DNA directed) polypeptide A, 220 kDa]; POLR2B [polymerase (RNA) II (DNA directed) polypeptide B, 140 kDa]; POLR2c [polymerase (RNA) II (DNA directed) polypeptide C, 33 kDa]; POLR2D [polymerase (RNA) II (DNA directed) polypeptide D]; POLR2E [polymerase (RNA) II (DNA directed) polypeptide E, 25 kDa]; POLR2F [polymerase (RNA) II (DNA directed) polypeptide F]; POLR2G [polymerase (RNA) II (DNA directed) polypeptide G]; POLR2H [polymerase (RNA) II (DNA directed) polypeptide H]; POLR2I [polymerase (RNA) 11 (DNA directed) polypeptide 1, 14.5 kDa]; POLR2J [polymerase (RNA) 11 (DNA directed) polypeptide J, 13.3 kDa]; POLR2K [polymerase (RNA) 1T (DNA directed) polypeptide K, 7.0 kDa]; POLR2L [polymerase (RNA) II (DNA directed) polypeptide L, 7.6 kDa]; POMC [proopiomelanocortin]; POMT1 [protein-O-mannosyltransferase 1]; PON1 [paraoxonase 1]; PON2 [paraoxonase 2]; PON3 [paraoxonase 3]; POSTN [periostin, osteoblast specific factor]; POT1 [POT1 protection of telomeres 1 homolog (S. pombe)]; POU2AF1 [POU class 2 associating factor 1]; POU2F1 [POU class 2 homeobox 1]; POU2F2 [POU class 2 homeobox 2]; POU5F1 [POU class 5 homeobox 1]; PPA1 [pyrophosphatase (inorganic) 1]; PPARA [peroxisome proliferator-activated receptor alpha]; PPARD [peroxisome proliferator-activated receptor delta]; PPARG [peroxisome proliferator-activated receptor gamma]; PPARGC1A [peroxisome proliferator-activated receptor gamma, coactivator 1 alpha]; PPAT [phosphoribosyl pyrophosphate amidotransferase]; PPBP [pro-platelet basic protein (chemokine (C-X-C motif) ligand 7)]; PPFIA1 [protein tyrosine phosphatase, receptor type, fpolypeptide (PTPRF), interacting protein (liprin), alpha 1]; PPIA [peptidylprolyl isomerase A (cyclophilin A)]; PPIB [peptidylprolyl isomerase B (cyclophilin B)]; PPIG [peptidylprolyl isomerase G (cyclophilin G)]; PPDX [protoporphyrinogen oxidase]; PPP1CB [protein phosphatase 1, catalytic subunit, beta isozyme]; PPP1R12A [protein phosphatase 1, regulatory (inhibitor) subunit 12A]; PPP1R2 [protein phosphatase 1, regulatory (inhibitor) subunit 2]; PPP2R1B [protein phosphatase 2, regulatory subunit A, beta]; PPP2R2B [protein phosphatase 2, regulatory subunit B, beta]; PPP2R4 [protein phosphatase 2A activator, regulatory subunit 4]; PPP6C [protein phosphatase 6, catalytic subunit]; PPT1 [palmitoyl-protein thioesterase 1]; PPY [pancreatic polypeptide]; PRDM1 [PR domain containing 1, with ZNF domain]; PRDM2 [PR domain containing 2, with ZNF domain]; PRDX2 [peroxiredoxin2]; PRDX3 [peroxiredoxin 3]; PRDX5 [peroxiredoxin 5]; PRF1 [perforin 1 (pore forming protein)]; PRG2 [proteoglycan 2, bone marrow (natural killer cell activator, eosinophil granule major basic protein)]; PRG4 [proteoglycan4]; PRIM1 [primase, DNA, polypeptide 1 (49 kDa)]; PRKAA1 [protein kinase, AMP-activated, alpha 1 catalytic subunit]; PRKAA2 [protein kinase, AMP-activated, alpha 2 catalytic subunit]; PRKAB 1 [protein kinase, AMP-activated, beta 1 non-catalytic subunit]; PRKACA [protein kinase, cAMP-dependent, catalytic, alpha]; PRKACB [protein kinase, cAMP-dependent, catalytic, beta]; PRKACG [protein kinase, cAMP-dependent, catalytic, gamma]; PRKAR1A [protein kinase, cAMP-dependent, regulatory, type I, alpha (tissue specific extinguisher 1)]; PRKAR2A [protein kinase, cAMP-dependent, regulatory, type II, alpha]; PRKAR2B [protein kinase, cAMP-dependent, regulatory, type II, beta]; PRKCA [protein kinase C, alpha]; PRKCB [protein kinase C, beta]; PRKCD [protein kinase C, delta]; PRKCE [protein kinase C, epsilon]; PRKCG [protein kinase C, gamma]; PRKCH [protein kinase C, eta]; PRKCI [protein kinase C, iota]; PRKCQ [protein kinase C, theta]; PRKCZ [protein kinase C, zeta]; PRKD1 [protein kinase D1]; PRKD3 [protein kinase D3]; PRKDC [protein kinase, DNA-activated, catalytic polypeptide; also known as DNAPK]; PRKG1 [protein kinase, cGMP-dependent, type I]; PRKRIR [protein-kinase, interferon-inducible double stranded RNA dependent inhibitor, repressor of (P58 repressor)]; PRL [prolactin]; PRLR [prolactin receptor]; PRNP [prion protein]; PROC [protein C (inactivator of coagulation factors Va and VIIIa)]; PRODH [proline dehydrogenase (oxidase) 1]; PROK1 [prokineticin 1]; PROK2 [prokineticin 2]; PROM1 [prominin 1]; PR051 [proteinS (alpha)]; PRPH [peripherin]; PRSS1 [protease, serine, 1 (trypsin 1)]; PRSS2 [protease, serine, 2 (trypsin 2)]; PRSS21 [protease, serine, 21 (testisin)]; PRSS3 [protease, serine, 3]; PRTN3 [proteinase 3]; PSAP [prosaposin]; PSEN1 [presenilin 1]; PSEN2 [presenilin 2 (Alzheimer disease 4)]; PSMA1 [proteasome (prosome, macropain) subunit, alpha type, 1]; PSMA2 [proteasome (prosome, macropain) subunit, alpha type, 2]; PSMA3 [proteasome (prosome, macropain) subunit, alpha type, 3]; PSMA5 [proteasome (prosome, macropain) subunit, alpha type, 5]; PSMA6 [proteasome (prosome, macropain) subunit, alpha type, 6]; PSMA7 [proteasome (prosome, macropain) subunit, alpha type, 7]; PSMB10 [proteasome (prosome, macropain) subunit, beta type, 10]; PSMB2 [proteasome (prosome, macropain) subunit, beta type, 2]; PSMB4 [proteasome (prosome, macropain) subunit, beta type, 4]; PSMB5 [proteasome (prosome, macropain) subunit, beta type, 5]; PSMB6 [proteasome (prosome, macropain) subunit, beta type, 6]; PSMB8 [proteasome (prosome, macropain) subunit, beta type, R (large multifunctional peptidase 7)]; PSMB9 [proteasome (prosome, macropain) subunit, beta type, 9 (large multifunctional peptidase 2)]; PSMC3 [proteasome (prosome, macropain) 26S subunit, ATPase, 3]; PSMC4 [proteasome (prosome, macropain) 26S subunit, ATPase, 4]; PSMC6 [proteasome (prosome, macropain) 26S subunit, ATPase, 6]; PSMD4 [proteasome (prosome, macropain) 26S subunit, non-ATPase, 4]; PSMD9 [proteasome (prosome, macropain) 26S subunit, non-ATPase, 9]; PSME1 [proteasome (prosome, macropain) activator subunit 1 (PA28 alpha)]; PSME3 [proteasome (prosome, macropain) activator subunit 3 (PA28 gamma; Ki)]; PSMG2 [proteasome (prosome, macropain) assembly chaperone 2]; PSORS1C1 [psoriasis susceptibility 1 candidate 1]; PSTPIP1 [proline-serine-threonine phosphatase interacting protein 1]; PTAFR [platelet-activating factor receptor]; PTBP1 [polypyrimidine tract binding protein 1]; PTCH1 [patched homolog 1 (Drosophila)]; PTEN [phosphatase and tensin homolog]; PTGDR [prostaglandin D2 receptor (DP)]; PTGDS [prostaglandin D2 synthase 21 kDa (brain)]; PTGER1 [prostaglandin E receptor 1 (subtype EP1), 42 kDa]; PTGER2 [prostaglandin E receptor 2 (subtype EP2), 53 kDa]; PTGER3 [prostaglandin E receptor 3 (subtype EP3)]; PTGER4 [prostaglandin E receptor 4 (subtype EP4)]; PTGES [prostaglandin E synthase]; PTGFR [prostaglandin F receptor (FP)]; PTGIR [prostaglandin 12 (prostacyclin) receptor (IP)]; PTGS 1 [prostaglandin-endoperoxide synthase 1 (prostaglandin G/H synthase and cyclooxygenase)]; PTGS2 [prostaglandin-endoperoxide synthase 2 (prostaglandin G/H synthase and cyclooxygenase)]; PTH [parathyroid hormone]; PTHLH [parathyroid hormone-like hormone]; PTK2 [PTK2 protein tyrosine kinase 2]; PTK2B [PTK2B protein tyrosine kinase 2 beta]; PTK7 [PTK7 protein tyrosine kinase 7]; PTMS [parathymosin]; PTN [pleiotrophin]; PTPN1 [protein tyrosine phosphatase, non-receptor type 1]; PTPN11 [protein tyrosine phosphatase, non-receptor type 11]; PTPN12 [protein tyrosine phosphatase, non-receptor type 12]; PTPN2 [protein tyrosine phosphatase, non-receptor type 2]; PTPN22 [protein tyrosine phosphatase, non-receptor type 22 (lymphoid)]; PTPN6 [protein tyrosine phosphatase, non-receptor type 6]; PTPRC [protein tyrosine phosphatase, receptor type, C]; PTPRD [protein tyrosine phosphatase, receptor type, D]; PTPRE [protein tyrosine phosphatase, receptor type, E]; PTPRJ [protein tyrosine phosphatase, receptor type, J]; PTPRN [protein tyrosine phosphatase, receptor type, N]; PTPRT [protein tyrosine phosphatase, receptor type, T]; PTPRU [protein tyrosine phosphatase, receptor type, U]; PTRF [polymerase 1 and transcript release factor]; PTS [6-pyruvoyltetrahydropterin synthase]; PTTG1 [pituitary tumor-transforming 1]; PTX3 [pentraxin 3, long]; PUS10 [pseudouridylate synthase 10]; PXK [PX domain containing serine/threonine kinase]; PXN [paxillin]; PYCR1 [pyrroline-5-carboxylate reductase 1]; PYCR2 [pyrroline-5-carboxylate reductase family, member 2]; PYGB [phosphorylase, glycogen; brain]; PYGM [phosphorylase, glycogen, muscle]; PYY [peptide YY]; PZP [pregnancy-zone protein]; QDPR [quinoid dihydropteridine reductase]; RAB11 A [RAB11A, member RAS oncogene family]; RAB11FIP1 [RAB11 family interacting protein 1 (class I)]; RAB27A [RAB27A, member RAS oncogene family]; RAB37 [RAB37, member RAS oncogene family]; RAB39 [RAB39, member RAS oncogene family]; RAB7A [RAB7A, member RAS oncogene family]; RAB9A [RAB9A, member RAS oncogene family]; RAC1 [ras-related C3 botulinum toxin substrate 1 (rho family, small GTP binding protein Racl)]; RAC2 [ras-related C3 botulinum toxin substrate 2 (rho family, small GTP binding protein Rac2)]; RAD17 [RAD17 homolog (S. pombe)]; RAD50 [RAD50 homolog (S. cerevisiae)]; RAD51 [RAD51 homolog (RecA homolog, E. coli) (S. cerevisiae)]; RAD51C [RAD51 homolog C (S. cerevisiae)]; RAD51L1 [RAD51-like 1 (S. cerevisiae)]; RAD51L3 [RAD51-like 3 (S. cerevisiae)]; RAD54L [RAD54-like (S. cerevisiae)]; RAD9A [RAD9 homolog A (S. pombe)]; RAF1 [v-raf-1 murine leukemia viral oncogene homolog 1]; RAG1 [recombination activating gene 1]; RAC2 [recombination activating gene 2]; RAN [RAN, member RAS oncogene family]; RANBP1 [RAN binding protein 1]; RAP1A [RAP1A, member of RAS oncogene family]; RAPGEF4 [Rap guanine nucleotide exchange factor (GEF) 4]; RARA [retinoic acid receptor, alpha]; RARB [retinoic acid receptor, beta]; RARG [retinoic acid receptor, gamma]; RARRES2 [retinoic acid receptor responder (tazarotene induced) 2]; RARS [arginyl-tRNA synthetase]; RASA1 [RAS p21 protein activator (GTPase activating protein) 1]; RASGRP1 [RAS guanyl releasing protein 1 (calcium and DAG-regulated)]; RASGRP2 [RAS guanyl releasing protein 2 (calcium and DAG-regulated)]; RASGRP4 [RAS guanyl releasing protein 4]; RASSF1 [Ras association (RalGDS/AF-6) domain family member 1]; RB1 [retinoblastoma 1]; RBBP4 [retinoblastoma binding protein 4]; RBBP8 [retinoblastoma binding protein 8]; RBL1 [retinoblastoma-like 1 (p107)]; RBL2 [retinoblastoma-like 2 (p130)]; RBP4 [retinol binding protein 4, plasma]; RBX1 [ring-box 1]; RCBTB1 [regulator of chromosome condensation (RCC1) and BTB (POZ) domain containing protein 1]; RCN1 [reticulocalbin 1, EF-hand calcium binding domain]; RCN2 [reticulocalbin 2, EF-hand calcium binding domain]; RDX [radixin]; RECK [reversion-inducing-cysteine-rich protein with kazal motifs]; RECQL [RecQ protein-like (DNA helicase Q1-like)]; RECQL4 [RecQ protein-like 4]; RECQL5 [RecQ protein-like 5]; REG1A [regenerating islet-derived 1 alpha]; REG3A [regenerating islet-derived 3 alpha]; REG4 [regenerating islet-derived family, member 4]; REL [v-rel reticuloendotheliosis viral oncogene homolog (avian)]; RELA [v-rel reticuloendotheliosis viral oncogene homolog A (avian)]; RELB [v-rel reticuloendotheliosis viral oncogene homolog B]; REN [renin]; RET [ret proto-oncogene]; RETN [resistin]; RETNLB [resistin like beta]; RFC1 [replication factor C (activator 1) 1, 145 kDa]; RFC2 [replication factor C (activator 1) 2, 40 kDa]; RFC3 [replication factor C (activator 1) 3, 38 kDa]; RFX1 [regulatory factor X, 1 (influences HLA class 11 expression)]; RFX5 [regulatory factor X, 5 (influences HLA class 1T expression)]; RFXANK [regulatory factor X-associated ankyrin-containing protein]; RFXAP [regulatory factor X-associated protein]; RGS 18 [regulator of G-protein signaling 18]; RHAG [Rh-associated glycoprotein]; RHO [Rh blood group, D antigen]; RHO [rhodopsin]; RHOA [ras homolog gene family, member A]; RHOD [ras homolog gene family, member D]; RIF1 [RAP1 interacting factor homolog (yeast)]; RIPK1 [receptor (TNFRSF)-interacting serine-threonine kinase 1]; RIPK2 [receptor-interacting serine-threonine kinase 2]; RLBP1 [retinaldehyde binding protein 1]; RLN1 [relaxin 1]; RLN2 [relaxin 2]; RMT1 [RMi1, RecQ mediated genome instability 1, homolog (S. cerevisiae)]; RNASE1 [ribonuclease, RNase A family, 1 (pancreatic)]; RNASE2 [ribonuclease, RNase A family, 2 (liver, eosinophil-derived neurotoxin)]; RNASE3 [ribonuclease, RNase A family, 3 (eosinophil cationic protein)]; RNASEH1 [ribonuclease H1]; RNASEH2A [ribonuclease H2, subunit A]; RNASEL [ribonuclease L (2′ [5′-oligoisoadenylate synthetase-dependent)]; RNASEN [ribonuclease type III, nuclear]; RNF123 [ring finger protein 123]; RNF13 [ring finger protein 13]; RNF135 [ring finger protein 135]; RNF138 [ring finger protein 138]; RNF4 [ring finger protein 4]; RNH1 [ribonuclease/angiogenin inhibitor 1]; RNPC3 [RNA-binding region (RNP1, RRM) containing 3]; RNPEP [arginyl aminopeptidase (aminopeptidase B)]; ROCK1 [Rho-associated, coiled-coil containing protein kinase 1]; ROM1 [retinal outer segment membrane protein 1]; ROR2 [receptor tyrosine kinase-like orphan receptor 2]; RORA [RAR-related orphan receptor A]; RPA1 [replication protein A1, 70 kDa]; RPA2 [replication protein A2, 32 kDa]; RPGRIP1L [RPGRIPI-like]; RPLP1 [ribosomal protein, large, P1]; RPS19 [ribosomal protein S19]; RPS6KA3 [ribosomal protein S6 kinase, 90 kDa, polypeptide 3]; RPS6KB1 [ribosomal protein S6 kinase, 70 kDa, polypeptide 1]; RPSA [ribosomal protein SA]; RRBP1 [ribosome binding protein 1 homolog 180 kDa (dog)]; RRM1 [ribonucleotide reductase M1]; RRM2B [ribonucleotide reductase M2B (TP53 inducible)]; RUNX1 [runt-related transcription factor 1]; RUNX3 [runt-related transcription factor 3]; RXRA [retinoid X receptor, alpha]; RXRB [retinoid X receptor, beta]; RYR1 [ryanodine receptor 1 (skeletal)]; RYR3 [ryanodine receptor 3]; S100A1 [S100 calcium binding protein A1]; S100A12 [S100 calcium binding protein A12]; S100A4 [S100 calcium binding protein A4]; S100A7 [S100 calcium binding protein A7]; S100A8 [S 100 calcium binding protein A8]; S100A9 [S 100 calcium binding protein A9]; S 100B [S100 calcium binding protein B]; S100G [S100 calcium binding protein G]; S1PR1 [sphingosine-1-phosphate receptor 1]; SAA1 [serum amyloid A1]; SAA4 [serum amyloid A4, constitutive]; SAFB [scaffold attachment factor B]; SAG [S-antigen; retina and pineal gland (arrestin)]; SAGE1 [sarcoma antigen 1]; SARDH [sarcosine dehydrogenase]; SART3 [squamous cell carcinoma antigen recognized by T cells 3]; SBDS [Shwachman-Bodian-Diamond syndrome]; SBNO2 [strawberry notch homolog 2 (Drosophila)]; SCAMP3 [secretory carrier membrane protein 3]; SOAP [SREBF chaperone]; SCARB1 [scavenger receptor class B, member 1]; SCD [stearoyl-CoA desaturase (delta-9-desaturase)]; SCG2 [secretogranin II]; SCG3 [secretogranin III]; SCG5 [secretogranin V (7B2 protein)]; SCGB1A1 [secretoglobin, family 1A, member 1 (uteroglobin)]; SCGB3A2 [secretoglobin, family 3A, member 2]; SCN4A [sodium channel, voltage-gated, type N, alpha subunit]; SCNN1A [sodium channel, nonvoltage-gated 1 alpha]; SCNN1G [sodium channel, nonvoltage-gated 1, gamma]; SCO1 [SCO cytochrome oxidase deficient homolog 1 (yeast)]; SC02 [SCO cytochrome oxidase deficient homolog 2 (yeast)]; SCP2 [sterol carrier protein 2]; SCT [secretin]; SDC1 [syndecan 1]; SDC2 [syndecan 2]; SDC4 [syndecan 4]; SDHB [succinate dehydrogenase complex, subunit B, iron sulfur (Ip)]; SDHD [succinate dehydrogenase complex, subunit D, integral membrane protein]; SEC14L2 [SEC14-like 2 (S. cerevisiae)]; SEC16A [SEC16 homolog A (S. cerevisiae)]; SEC23B [Sec23 homolog B (S. cerevisiae)]; SELE [selectin E]; SELL [selectin L]; SELP [selectin P (granule membrane protein 140 kDa, antigen CD62)]; SELPLG [selectin P ligand]; SEPT5 [septin 5]; SEPP1 [selenoprotein P, plasma, 1]; SEPSECS [Sep (0-phosphoserine) tRNA:Sec (selenocysteine) tRNA synthase]; SERBP1 [SERPINE1 mRNA binding protein 1]; SERPINA1 [serpin peptidase inhibitor, clade A (alpha-1 antiproteinase, antitrypsin), member 1]; SERPINA2 [serpin peptidase inhibitor, clade A (alpha-1 antiproteinase, antitrypsin), member 2]; SERPINA3 [serpin peptidase inhibitor, clade A (alpha-1 antiproteinase, antitrypsin), member 3]; SERPINA5 [serpin peptidase inhibitor, clade A (alpha-1 antiproteinase, antitrypsin), member 5]; SERPINA6 [serpin peptidase inhibitor, clade A (alpha-1 antiproteinase, antitrypsin), member 6]; SERPINA7 [serpin peptidase inhibitor, clade A (alpha-1 antiproteinase, antitrypsin), member 7]; SERPINB1 [serpin peptidase inhibitor, clade B (ovalbumin), member 1]; SERPINB2 [serpin peptidase inhibitor, clade B (ovalbumin), member 2]; SERPINB3 [serpin peptidase inhibitor, clade B (ovalbumin), member 3]; SERPINB4 [serpin peptidase inhibitor, clade B (ovalbumin), member 4]; SERPINB5 [serpin peptidase inhibitor, clade B (ovalbumin), member 5]; SERPINB6 [serpin peptidase inhibitor, clade B (ovalbumin), member 6]; SERPINB9 [serpin peptidase inhibitor, clade B (ovalbumin), member 9]; SERPINC1 [serpin peptidase inhibitor, clade C (antithrombin), member 1]; SERPIND1 [serpin peptidase inhibitor, clade D (heparin cofactor), member 1]; SERPINE1 [serpin peptidase inhibitor, clade E (nexin, plasminogen activator inhibitor type 1), member 1]; SERPINE2 [serpin peptidase inhibitor, clade E (nexin, plasminogen activator inhibitor type 1), member 2]; SERPINF2 [serpin peptidase inhibitor, clade F (alpha-2 antiplasmin, pigment epithelium derived factor), member 2]; SERPING1 [serpin peptidase inhibitor, clade G (C1 inhibitor), member 1]; SERPINH1 [serpin peptidase inhibitor, clade H (heat shock protein 47), member 1, (collagen binding protein 1)]; SET [SET nuclear oncogene]; SETDB2 [SET domain, bifurcated 2]; SETX [senataxin]; SFPQ [splicing factor proline/glutamine-rich (polypyrimidine tract binding protein associated)]; SFRP1 [secreted frizzled-related protein 1]; SFRP2 [secreted frizzled-related protein 2]; SFRP5 [secreted frizzled-related protein 5]; SFTPA1 [surfactant protein A1]; SFTPB [surfactant protein B]; SFTPC [surfactant protein C]; SFTPD [surfactant protein D]; SGCA [sarcoglycan, alpha (50 kDa dystrophin-associated glycoprotein)]; SGCB [sarcoglycan, beta (43 kDa dystrophin-associated glycoprotein)]; SGK1 [serum/glucocorticoid regulated kinase 1]; SGSH [N-sulfoglucosamine sulfohydrolase]; SGTA [small glutamine-rich tetratricopeptide repeat (TPR)-containing, alpha]; SH2B 1 [SH2B adaptor protein 1]; SH2B3 [SH2B adaptor protein 3]; SH2D1A [SH2 domain containing 1A]; SH2D4B [SH2 domain containing 4B]; SH3KBP1 [SH3-domain kinase binding protein 1]; SHBG [sex hormone-binding globulin]; SHC1 [SHC (Src homology 2 domain containing) transforming protein 1]; SHH [sonic hedgehog homolog (Drosophila)]; SHMT2 [serine hydroxymethyltransferase 2 (mitochondrial)]; S1 [sucrase-isomaltase (alpha-glucosidase)]; STGTRR [single immunoglobulin and toll-interleukin 1 receptor (TTR) domain]; STP1 [survival of motor neuron protein interacting protein 1]; SIPA1 [signal-induced proliferation-associated 1]; SIRPA [signal-regulatory protein alpha]; SIRPB2 [signal-regulatory protein beta 2]; SIRT1 [sirtuin (silent mating type information regulation 2 homolog) 1 (S. cerevisiae)]; SKIV2L [superkiller viralicidic activity 2-like (S. cerevisiae)]; SKP2 [S-phase kinase-associated protein 2 (p45)]; SLAMF1 [signaling lymphocytic activation molecule family member 1]; SLAMF6 [SLAM family member 6]; SLC11 A 1 [solute carrier family 11 (proton-coupled divalent metal ion transporters), member 1]; SLC11A2 [solute carrier family 11 (proton-coupled divalent metal ion transporters), member 2]; SLC12A1 [solute carrier family 12 (sodium/potassium/chloride transporters), member 1]; SLC12A2 [solute carrier family 12 (sodium/potassium/chloride transporters), member 2]; SLC14A1 [solute carrier family 14 (urea transporter), member 1 (Kidd blood group)]; SLC15A1 [solute carrier family 15 (oligopeptide transporter), member 1]; SLC16A1 [solute carrier family 16, member 1 (monocarboxylic acid transporter 1)]; SLC17A5 [solute carrier family 17 (anion/sugar transporter), member 5]; SLC17A6 [solute carrier family 17 (sodium-dependent inorganic phosphate cotransporter), member 6]; SLC17A7 [solute carrier family 17 (sodium-dependent inorganic phosphate cotransporter), member 7]; SLC19A1 [solute carrier family 19 (folate transporter), member 1]; SLC1A1 [solute carrier family 1 (neurona1'epithelial high affinity glutamate transporter, system Xag), member 1]; SLC1A2 [solute carrier family 1 (glial high affinity glutamate transporter), member 2]; SLC1A4 [solute carrier family 1 (glutamate/neutral amino acid transporter), member 4]; SLC22A12 [solute carrier family 22 (organic anion/urate transporter), member 12]; SLC22A2 [solute carrier family 22 (organic cation transporter), member 2]; SLC22A23 [solute carrier family 22, member 23]; SLC22A3 [solute carrier family 22 (extraneuronal monoamine transporter), member 3]; SLC22A4 [solute carrier family 22 (organic cation/ergothioneine transporter), member 4]; SLC22A5 [solute carrier family 22 (organic cation/camitine transporter), member 5]; SLC22A6 [solute carrier family 22 (organic anion transporter), member 6]; SLC24A2 [solute carrier family 24 (sodium/potassium/calcium exchanger), member 2]; SLC25A1 [solute carrier family 25 (mitochondrial carrier; citrate transporter), member 1]; SLC25A20 [solute carrier family 25 (camitine/acylcamitine translocase), member 20]; SLC25A3 [solute carrier family 25 (mitochondrial carrier; phosphate carrier), member 3]; SLC25A32 [solute carrier family 25, member 32]; SLC25A33 [solute carrier family 25, member 33]; SLC25A4 [solute carrier family 25 (mitochondrial carrier; adenine nucleotide translocator), member 4]; SLC26A4 [solute carrier family 26, member 4]; SLC27A4 [solute carrier family 27 (fatty acid transporter), member 4]; SLC28A1 [solute carrier family 28 (sodium-coupled nucleoside transporter), member 1]; SLC2A1 [solute carrier family 2 (facilitated glucose transporter), member 1]; SLC2A13 [solute carrier family 2 (facilitated glucose transporter), member 13]; SLC2A3 [solute carrier family 2 (facilitated glucose transporter), member 3]; SLC2A4 [solute carrier family 2 (facilitated glucose transporter), member 4]; SLC30A1 [solute carrier family 30 (zinc transporter), member 1]; SLC30A8 [solute carrier family 30 (zinc transporter), member 8]; SLC31A1 [solute carrier family 31 (copper transporters), member 1]; SLC35A1 [solute carrier family 35 (CMP-sialic acid transporter), member A1]; SLC35A2 [solute carrier family 35 (UDP-galactose transporter), member A2]; SLC35C1 [solute carrier family 35, member C1]; SLC35F2 [solute carrier family 35, member F2]; SLC39A3 [solute carrier family 39 (zinc transpmier), member 3]; SLC3A2 [solute carrier family 3 (activators of dibasic and neutral amino acid transport), member 2]; SLC46A1 [solute carrier family 46 (folate transporter), member 1]; SLC5A5 [solute carrier family 5 (sodium iodide symporter), member 5]; SLC6A11 [solute carrier family 6 (neurotransmitter transporter, GABA), member 11]; SLC6A14 [solute carrier family 6 (amino acid transporter), member 14]; SLC6A19 [solute carrier family 6 (neutral amino acid transporter), member 19]; SLC6A3 [solute carrier family 6 (neurotransmitter transporter, dopamine), member 3]; SLC6A4 [solute carrier family 6 (neurotransmitter transporter, serotonin), member 4]; SLC6A8 [solute carrier family 6 (neurotransmitter transpmier, creatine), member 8]; SLC7A1 [solute carrier family 7 (cationic amino acid transporter, y+ system), member 1]; SLC7A2 [solute carrier family 7 (cationic amino acid transporter, y+ system), member 2]; SLC7A4 [solute carrier family 7 (cationic amino acid transporter, y+ system), member 4]; SLC7AS [solute carrier family 7 (cationic amino acid transporter, y+ system), member 5]; SLC8A1 [solute carrier family 8 (sodium/calcium exchanger), member 1]; SLC9A1 [solute carrier family 9 (sodium/hydrogen exchanger), member 1]; SLC9A3R1 [solute carrier family 9 (sodium/hydrogen exchanger), member 3 regulator 1]; SLCO1A2 [solute carrier organic anion transporter family, member 1A2]; SLCO1B1 [solute carrier organic anion transporter family, member 1B1]; SLCO1B3 [solute carrier organic anion transporter family, member 1B3]; SLPI [secretory leukocyte peptidase inhibitor]; SMAD1 [SMAD family member 1]; SMAD2 [SMAD family member 2]; SMAD3 [SMAD family member 3]; SMAD4 [SMAD family member 4]; SMAD7 [SMAD family member 7]; SMARCA4 [SWI/SNF related, matrix associated, actin dependent regulator of chromatin, subfamily a, member 4]; SMARCAL1 [SWI/SNF related, matrix associated, actin dependent regulator of chromatin, subfamily a-like 1]; SMARCB1 [SWI/SNF related, matrix associated, actin dependent regulator of chromatin, subfamilyb, member 1]; SMC1A [structural maintenance of chromosomes 1A]; SMC3 [structural maintenance of chromosomes 3]; SMG1 [SMG1 homolog, phosphatidylinositol 3-kinase-related kinase (C. elegans)]; SMN1 [survival of motor neuron 1, telomeric]; SMPD1 [sphingomyelin phosphodiesterase 1, acid lysosomal]; SMPD2 [sphingomyelin phosphodiesterase 2, neutral membrane (neutral sphingomyelinase)]; SMTN [smoothelin]; SNAI2 [snail homolog 2 (Drosophila)]; SNAP25 [synaptosomal-associated protein, 25 kDa]; SNCA [synuclein, alpha (non A4 component of amyloid precursor)]; SNCG [synuclein, gamma (breast cancer-specific protein 1)]; SNURF [SNRPN upstream reading frame]; SNW1 [SNW domain containing 1]; SNX9 [sorting nexin 9]; SOAT1 [sterol 0-acyltransferase 1]; SOCS1 [suppressor of cytokine signaling 1]; SOCS2 [suppressor of cytokine signaling 2]; SOCS3 [suppressor of cytokine signaling 3]; SOD1 [superoxide dismutase 1, soluble]; SOD2 [superoxide dismutase 2, mitochondrial]; SORBS3 [sorbin and SH3 domain containing 3]; SORD [sorbitol dehydrogenase]; SOX2 [SRY (sex determining region Y)-box 2]; SP1 [Sp1 transcription factor]; SP110 [SP11 0 nuclear body protein]; SP3 [Sp3 transcription factor]; SPA17 [sperm autoantigenic protein 17]; SPARC [secreted protein, acidic, cysteine-rich (osteonectin)]; SPHK1 [sphingosine kinase 1]; SP11 [spleen focus forming virus (SFFV) proviral integration oncogene spil]; SP1NK1 [serine peptidase inhibitor, Kazal type I]; SPTNK13 [serine peptidase inhibitor, Kazal type 13 (putative)]; SPINK5 [serine peptidase inhibitor, Kazal type S]; SPN [sialophorin]; SPON1 [spondin 1, extracellular matrix protein]; SPP1 [secreted phosphoprotein 1]; SPRED1 [sprouty-related, EVH1 domain containing 1]; SPRR2A [small proline-rich protein 2A]; SPRR2B [small proline-rich protein 2B]; SPTB [spectrin, beta, erythrocytic]; SRC [v-src sarcoma (Schmidt-Ruppin A-2) viral oncogene homolog (avian)]; SRDSA1 [steroid-S-alpha-reductase, alpha polypeptide 1 (3-oxo-S alpha-steroid delta 4-dehydrogenase alpha 1)]; SREBF1 [sterol regulatory element binding transcription factor 1]; SREBF2 [sterol regulatory element binding transcription factor 2]; SRF [serum response factor (c-fos serum response element-binding transcription factor)]; SRGN [serglycin]; SRP9 [signal recognition particle 9 kDa]; SRPX [sushi-repeat-containing protein, X-linked]; SRR [serine racemase]; SRY [sex determining region Y]; SSB [Sjogren syndrome antigen B (autoantigen La)]; SST [somatostatin]; SSTR2 [somatostatin receptor 2]; SSTR4 [somatostatin receptor 4]; STRSIA4 [STR alpha-N-acetyl-neuraminide alpha-2,8-sialyltransferase 4]; STAR [steroidogenic acute regulatory protein]; STAT1 [signal transducer and activator of transcription 1, 91 kDa]; STAT2 [signal transducer and activator of transcription 2, 113 kDa]; STAT3 [signal transducer and activator of transcription 3 (acute-phase response factor)]; STAT4 [signal transducer and activator of transcription 4]; STATSA [signal transducer and activator of transcription SA]; STATSB [signal transducer and activator of transcription SB]; STAT6 [signal transducer and activator of transcription 6, interlenkin-4 induced]; STELLAR [germ and embryonic stem cell enriched protein STELLA]; STIM1 [stromal interaction molecule 1]; STIP1 [stress-induced-phosphoprotein 1]; STK11 [serine/threonine kinase 11]; STMN2 [tathmin-like 2]; STRAP [serine/threonine kinase receptor associated protein]; STRC [stereocilin]; STS [steroid sulfatase (microsomal), isozyme S]; STX6 [syntaxin 6]; STX8 [syntaxin 8]; SULT1A1 [sulfotransferase family, cytosolic, 1A, phenol-preferring, member 1]; SULT1A3 [sulfotransferase family, cytosolic, 1A, phenol-preferring, member 3]; SUMF1 [sulfatase modifying factor 1]; SUM01 [SMT3 suppressor of miftwo 3 homolog 1 (S. cerevisiae)]; SUM03 [SMT3 suppressor of miftwo 3 homolog 3 (S. cerevisiae)]; SUOX [sulfite oxidase]; SUV39H1 [suppressor of variegation 3-9 homolog 1 (Drosophila)]; SWAP70 [SWAP switching B-cell complex 70 kDa subunit]; SYCP3 [synaptonemal complex protein 3]; SYK [spleen tyrosine kinase]; SYNM [synemin, intermediate filament protein]; SYNPO [synaptopodin]; SYNPO2 [synaptopodin 2]; SYP [synaptophysin]; SYT3 [synaptotagmin III]; SYTL1 [synaptotagmin-like 1]; T [T, brachyury homolog (mouse)]; TAC1 [tachykinin, precursor 1]; TAC4 [tachykinin 4 (hemokinin)]; TACR1 [tachykinin receptor 1]; TACR2 [tachykinin receptor 2]; TACR3 [tachykinin receptor 3]; TAGLN [transgelin]; TAL1 [T-cell acute lymphocytic leukemia 1]; TAOK3 [TAO kinase 3]; TAP1 [transporter 1, ATP-binding cassette, sub-family B (MDR/TAP)]; TAP2 [transporter 2, ATP-binding cassette, sub-family B (MDR/TAP)]; TARDBP [TAR DNA binding protein]; TARP [TCR gamma alternate reading frame protein]; TAT [tyrosine aminotransferase]; TBK1 [TANK-binding kinase 1]; TBP [TATA box binding protein]; TBX1 [T-box 1]; TBX2 [T-box 2]; TBX21 [T-box 21]; TBX3 [T-box 3]; TBX5 [T-box 5]; TBXA2R [thromboxane A2 receptor]; TBXAS1 [thromboxane A synthase 1 (platelet)]; TCEA1 [transcription elongation factor A (S11), 1]; TCEAL1 [transcription elongation factor A (S11)-like 1]; TCF4 [transcription factor 4]; TCF7L2 [transcription factor 7-like 2 (T-cell specific, HMG-box)]; TCL1 A [T-cell leukemia/lymphoma 1A]; TCL1B [T-cellleukemia/lymphoma IB]; TCN1 [transcobalamin I (vitamin B12 binding protein, R binder family)]; TCN2 [transcobalamin II; macrocytic anemia]; TDP1 [tyrosyl-DNA phosphodiesterase 1]; TEC [tee protein tyrosine kinase]; TECTA [tectorin alpha]; TEK [TEK tyrosine kinase, endothelial]; TERF1 [telomeric repeat binding factor (NIMA-interacting) 1]; TERF2 [telomeric repeat binding factor 2]; TERT [telomerase reverse transcriptase]; TES [testis derived transcript (3 LTM domains)]; TF [transferrin]; TFAM [transcription factor A, mitochondrial]; TFAP2A [transcription factor AP-2 alpha (activating enhancer binding protein 2 alpha)]; TFF2 [trefoil factor 2]; TFF3 [trefoil factor 3 (intestinal)]; TFPI [tissue factor pathway inhibitor (lipoprotein-associated coagulation inhibitor)]; TFPT [TCF3 (E2A) fusion partner (in childhood Leukemia)]; TFR2 [transferrin receptor 2]; TFRC [transferrin receptor (p90, CD71)]; TG [thyroglobulin]; TGFA [transforming growth factor, alpha]; TGFB1 [transforming growth factor, beta 1]; TGFB2 [transforming growth factor, beta 2]; TGFB3 [transforming growth factor, beta 3]; TGFBR1 [transforming growth factor, beta receptor 1]; TGFBR2 [transforming growth factor, beta receptor II (70/80 kDa)]; TGIF1 [TGFB-induced factor homeobox 1]; TGM1 [transglutaminase 1 (K polypeptide epidermal type I, protein-glutamine-gamma-glutamyltransferase)]; TGM2 [transglutaminase 2 (C polypeptide, protein-glutamine-gamma-glutamyltransferase)]; TGM3 [transglutaminase 3 (E polypeptide, protein-glutamine-gamma-glutamyltransferase)]; TH [tyrosine hydroxylase]; THAP1 [TRAP domain containing, apoptosis associated protein 1]; THBD [thrombomodulin]; THBS1 [thrombospondin 1]; THBS3 [thrombospondin 3]; THPO [thrombopoietin]; THY1 [Thy-1 cell surface antigen]; TIA1 [TIA1 cytotoxic granule-associated RNA binding protein]; TIE1 [tyrosine kinase with immunoglobulin-like and EGF-like domains 1]; TIMD4 [T-cell immunoglobulin and mucin domain containing 4]; TIMELESS [timeless homolog (Drosophila)]; TIMP1 [TIMP metallopeptidase inhibitor 1]; TIMP2 [TIMP metallopeptidase inhibitor 2]; TIMP3 [TIMP metallopeptidase inhibitor 3]; TIRAP [toll-interleukin 1 receptor (TIR) domain containing adaptor protein]; TJP1 [tight junction protein 1 (zona occludens 1)]; TK1 [thymidine kinase 1, soluble]; TK2 [thymidine kinase 2, mitochondrial]; TKT [transketolase]; TLE4 [transducin-like enhancer of split 4 (E(sp1) homolog, Drosophila)]; TLR1 [toll-like receptor 1]; TLR1O [toll-like receptor 10]; TLR2 [toll-like receptor 2]; TLR3 [toll-like receptor 3]; TLR4 [toll-like receptor 4]; TLR5 [toll-like receptor 5]; TLR6 [toll-like receptor 6]; TLR7 [toll-like receptor 7]; TLR5 [toll-like receptor 8]; TLR9 [toll-like receptor 9]; TLX1 [T-cellleukemia homeobox 1]; TM7SF4 [transmembrane 7 superfamily member 4]; TMED3 [transmembrane emp24 protein transport domain containing 3]; TMEFF2 [transmembrane protein with EGF-like and two follistatin-like domains 2]; TMEM132E [transmembrane protein 132E]; TMEM18 [transmembrane protein 18]; TMEM19 [transmembrane protein 19]; TMEM216 [transmembrane protein 216]; TMEM27 [transmembrane protein 27]; TMEM67 [transmembrane protein 67]; TMPO [thymopoietin]; TMPRSS15 [transmembrane protease, serine 15]; TMSB4X [thymosin beta 4, X-linked]; TNC [tenascin C]; TNF [tumor necrosis factor (TNF superfamily, member 2)]; TNFAIP1 [tumor necrosis factor, alpha-induced protein 1 (endothelial)]; TNFAIP3 [tumor necrosis factor, alpha-induced protein 3]; TNFA1P6 [tumor necrosis factor, alpha-induced protein 6]; TNFRSF10A [tumor necrosis factor receptor superfamily, member 10a]; TNFRSF10B [tumor necrosis factor receptor superfamily, member 10b]; TNFRSF100 [tumor necrosis factor receptor superfamily, member 10c, decoy without an intracellular domain]; TNFRSF10D [tumor necrosis factor receptor superfamily, member 10d, decoy with truncated death domain]; TNFRSF11A [tumor necrosis factor receptor superfamily, member 11a, NFKB activator]; TNFRSF11B [tumor necrosis factor receptor superfamily, member 11b]; TNFRSF13B [tumor necrosis factor receptor superfamily, member 13B]; TNFRSF130 [tumor necrosis factor receptor superfamily, member 13C]; TNFRSF14 [tumor necrosis factor receptor superfamily, member 14 (herpesvirus entry mediator)]; TNFRSF17 [tumor necrosis factor receptor superfamily, member 17]; TNFRSF18 [tumor necrosis factor receptor superfamily, member 18]; TNFRSF1A [tumor necrosis factor receptor superfamily, member 1A]; TNFRSF1B [tumor necrosis factor receptor superfamily, member IB]; TNFRSF21 [tumor necrosis factor receptor superfamily, member 21]; TNFRSF25 [tumor necrosis factor receptor superfamily, member 25]; TNFRSF4 [tumor necrosis factor receptor superfamily, member 4]; TNFRSF6B [tumor necrosis factor receptor superfamily, member 6b, decoy]; TNFRSF8 [tumor necrosis factor receptor superfamily, member 8]; TNFRSF9 [tumor necrosis factor receptor superfamily, member 9]; TNFSF10 [tumor necrosis factor (ligand) superfamily, member 10]; TNFSF11 [tumor necrosis factor (ligand) superfamily, member 11]; TNFSF12 [tumor necrosis factor (ligand) superfamily, member 12]; TNFSF13 [tumor necrosis factor (ligand) superfamily, member 13]; TNFSF13B [tumor necrosis factor (ligand) superfamily, member 13b]; TNFSF14 [tumor necrosis factor (ligand) superfamily, member 14]; TNFSF15 [tumor necrosis factor (ligand) superfamily, member 15]; TNFSF18 [tumor necrosis factor (ligand) superfamily, member 18]; TNFSF4 [tumor necrosis factor (ligand) superfamily, member 4]; TNFSF8 [tumor necrosis factor (ligand) superfamily, member 8]; TNFSF9 [tumor necrosis factor (ligand) superfamily, member 9]; TNKS [tankyrase, TRF1-interacting ankyrin-related ADP-ribose polymerase]; TNNC1 [troponin C type 1 (slow)]; TNNI2 [troponin I type 2 (skeletal, fast)]; TNNI3 [troponin I type 3 (cardiac)]; TNNT3 [troponin T type 3 (skeletal, fast)]; TNP01 [transportin 1]; TNS1 [tensin 1]; TNXB [tenascin XB]; TOM1L2 [target of myb1-like 2 (chicken)]; TOP1 [topoisomerase (DNA) I]; TOP1MT [topoisomerase (DNA) I, mitochondrial]; TOP2A [topoisomerase (DNA) II alpha 170 kDa]; TOP2B [topoisomerase (DNA) II beta 180 kDa]; TOP3A [topoisomerase (DNA) III alpha]; TOPBP1 [topoisomerase (DNA) II binding protein 1]; TP53 [tumor protein p53]; TP53BP1 [tumor protein p53 binding protein 1]; TP53RK [TP53 regulating kinase]; TP63 [tumor protein p63]; TP73 [tumor protein p73]; TPD52 [tumor protein D52]; TPH1 [tryptophan hydroxylase 1]; TPi1 [triosephosphate isomerase 1]; TPM1 [tropomyosin 1 (alpha)]; TPM2 [tropomyosin 2 (beta)]; TPMT [thiopurine S-methyltransferase]; TPO [thyroid peroxidase]; TPP1 [tripeptidyl peptidase I]; TPP2 [tripeptidyl peptidase II]; TPPP [tubulin polymerization promoting protein]; TPPP3 [tubulin polymerization-promoting protein family member 3]; TPSAB1 [tryptase alpha/beta 1]; TPSB2 [tryptase beta 2 (gene/pseudogene)]; TPSD1 [ttyptase delta 1]; TPSG1 [tryptase gamma 1]; TPT1 [tumor protein, translationally-controlled 1]; TRADD [TNFRSF1A-associated via death domain]; TRAF1 [TNF receptor-associated factor 1]; TRAF2 [TNF receptor-associated factor 2]; TRAF31P2 [TRAF3 interacting protein 2]; TRAF6 [TN F receptor-associated factor 6]; TRATP [TRAF interacting protein]; TRAPPC1 0 [trafficking protein particle complex 10]; TRDN [triadin]; TREX1 [three prime repair exonuclease 1]; TRH [thyrotropin-releasing hormone]; TRIB1 [tribbles homolog 1 (Drosophila)]; TRIM21 [tripartite motif-containing 21]; TRIM22 [tripartite motif-containing 22]; TRIM26 [tripartite motif-containing 26]; TRIM28 [tripartite motif-containing 28]; TRIM29 [tripartite motif-containing 29]; TRIM68 [tripartite motif-containing 68]; TRPA1 [transient receptor potential cation channel, subfamily A, member 1]; TRPC1 [transient receptor potential cation channel, subfamily C, member 1]; TRPC3 [transient receptor potential cation channel, subfamily C, member 3]; TRPC6 [transient receptor potential cation channel, subfamily C, member 6]; TRPM1 [transient receptor potential cation channel, subfamily M, member 1]; TRPM8 [transient receptor potential cation channel, subfamily M, member 8]; TRPS1 [trichorhinophalangeal syndrome I]; TRPV1 [transient receptor potential cation channel, subfamily V, member 1]; TRPV4 [transient receptor potential cation channel, subfamily V, member 4]; TRPV5 [transient receptor potential cation channel, subfamily V, member 5]; TRPV6 [transient receptor potential cation channel, subfamily V, member 6]; TRRAP [transformation/transcription domain-associated protein]; TSC1 [tuberous sclerosis 1]; TSC2 [tuberous sclerosis 2]; TSC22D3 [TSC22 domain family, member 3]; TSG101 [tumor susceptibility gene 101]; TSHR [thyroid stimulating hormone receptor]; TSLP [thymic stromal lymphopoietin]; TSPAN7 [tetraspanin 7]; TSPO [translocatorprotein (18 kDa)]; TSSK2 [testis-specific serine kinase 2]; TSTA3 [tissue specific transplantation antigen P35B]; TTF2 [transcription termination factor, RNA polymerase II]; TTN [titin]; TTPA [tocopherol (alpha) transfer protein]; TTR [transthyretin]; TUBAIB [tubulin, alpha 1b]; TUBA4A [tubulin, alpha4a]; TUBB [tubulin, beta]; TUBB1 [tubulin, beta 1]; TUBG1 [tubulin, gamma 1]; TWIST1 [twist homolog 1 (Drosophila)]; TWSG1 [twisted gastrulation homolog 1 (Drosophila)]; TXK [TXK tyrosine kinase]; TXN [thioredoxin]; TXN2 [thioredoxin 2]; TXNDC5 [thioredoxin domain containing 5 (endoplasmic reticulum)]; TXNDC9 [thioredoxin domain containing 9]; TXNIP [thioredoxin interacting protein]; TXNRD1 [thioredoxin reductase 1]; TXNRD2 [thioredoxin reductase 2]; TYK2 [tyrosine kinase 2]; TYMP [thymidine phosphorylase]; TYMS [thymidylate synthetase]; TYR [tyrosinase (oculocutaneous albinism 1A)]; TYR03 [TYR03 protein tyrosine kinase]; TYROBP [TYRO protein tyrosine kinase binding protein]; TYRP1 [tyrosinase-related protein 1]; UBB [ubiquitin B]; UBC [ubiquitin C]; UBE2C [ubiquitin-conjugating enzyme E2C]; UBE2N [ubiquitin-conjugating enzyme E2N (UBC13 homolog, yeast)]; UBE2U [ubiquitin-conjugating enzyme E2U (putative)]; UBE3A [ubiquitin protein ligase E3A]; UBE4A [ubiquitination factor E4A (UFD2 homolog, yeast)]; UCHL1 [ubiquitin carboxyl-terminal esterase L1 (ubiquitin thiolesterase)]; UCN [urocortin]; UCN2 [urocortin 2]; UCP1 [uncoupling protein 1 (mitochondrial, proton carrier)]; UCP2 [uncoupling protein 2 (mitochondrial, proton carrier)]; UCP3 [uncoupling protein 3 (mitochondrial, proton carrier)]; UFD1L [ubiquitin fusion degradation 1 like (yeast)]; UGCG [UDP-glucose ceramide glucosyltransferase]; UGP2 [UDP-glucose pyrophosphorylase 2]; UGT1A1 [UDP glucuronosyltransferase 1 family, polypeptide A1]; UGT1A6 [UDP glucuronosyltransferase 1 family, polypeptide A6]; UGT1A7 [UDP glucuronosyltransferase 1 family, polypeptide A7]; UGT8 [UDP glycosyltransferase 8]; U1MC1 [ubiquitin interaction motif containing 1]; ULBP1 [UL16 binding protein 1]; ULK2 [unc-51-like kinase 2 (C. elegans)]; UMOD [uromodulin]; UMPS [uridine monophosphate synthetase]; UNC13D [unc-13 homolog D (C. elegans)]; UNC93B1 [unc-93 homolog B1 (C. elegans)]; UNG [uracil-DNA glycosylase]; UQCRFS1 [ubiquinol-cytochrome c reductase, Rieske iron-sulfur polypeptide 1]; UROD [uroporphyrinogen decarboxylase]; USF1 [upstream transcription factor 1]; USF2 [upstream transcription factor 2, c-fos interacting]; USP18 [ubiquitin specific peptidase 18]; USP34 [ubiquitin specific peptidase 34]; UTRN [utrophin]; UTS2 [urotensin 2]; VAMPS [vesicle-associated membrane protein 8 (endobrevin)]; VAPA [VAMP (vesicle-associated membrane protein)-associated protein A, 33 kDa]; VASP [vasodilator-stimulated phosphoprotein]; VAV1 [vav 1 guanine nucleotide exchange factor]; VAV3 [vav 3 guanine nucleotide exchange factor]; VCAM1 [vascular cell adhesion molecule 1]; VCAN [versican]; VCL [vinculin]; VDAC1 [voltage-dependent anion channel 1]; VDR [vitamin D (1 [25-dihydroxyvitamin D3) receptor]; VEGFA [vascular endothelial growth factor A]; VEGFC [vascular endothelial growth factor C]; VHL [von Rippel-Lindau tumor suppressor]; VIL1 [villin 1]; VIM [vimentin]; VIP [vasoactive intestinal peptide]; VIPR1 [vasoactive intestinal peptide receptor 1]; VIPR2 [vasoactive intestinal peptide receptor 2]; VLDLR [very low density lipoprotein receptor]; VMAC [vimentin-type intermediate filament associated coiled-coil protein]; VPREB1 [pre-B lymphocyte 1]; VPS39 [vacuolar protein sorting 39 homolog (S. cerevisiae)]; VTN [vitronectin]; VWF [von Willebrand factor]; WARS [tryptophanyl-tRNA synthetase]; WAS [Wiskott-Aldrich syndrome (eczema-thrombocytopenia)]; WASF1 [WAS protein family, member 1]; WASF2 [WAS protein family, member 2]; WASL [Wiskott-Aldrich syndrome-like]; WDFY3 [WD repeat and FYVE domain containing 3]; WDR36 [WD repeat domain 36]; WEE1 [WEE1 homolog (S. pombe)]; WIF1 [WNT inhibitory factor 1]; WIPF1 [WAS/WASL interacting protein family, member 1]; WNK1 [WNK lysine deficient protein kinase 1]; WNT5A [wingless-type MMTV integration site family, member 5A]; WRN [Werner syndrome, RecQ helicase-like]; WT1 [Wilms tumor 1]; XBP1 [X-box binding protein 1]; XCL1 [chemokine (C motif) ligand 1]; XDH [xanthine dehydrogenase]; XIAP [X-linked inhibitor of apoptosis]; XPA [xeroderma pigmentosum, complementation group A]; XPC [xerodetma pigmentosum, complementation group C]; XP05 [exportin 5]; XRCC1 [X-ray repair complementing defective repair in Chinese hamster cells 1]; XRCC2 [X-ray repair complementing defective repair in Chinese hamster cells 2]; XRCC3 [X-ray repair complementing defective repair in Chinese hamster cells 3]; XRCC4 [X-ray repair complementing defective repair in Chinese hamster cells 4]; XRCC5 [X-ray repair complementing defective repair in Chinese hamster cells 5 (double-strand-break rejoining)]; XRCC6 [X-ray repair complementing defective repair in Chinese hamster cells 6]; YAP1 [Yes-associated protein 1]; YARS [tyrosyl-tRNA synthetase]; YBX1 [Y box binding protein 1]; YES 1 [v-yes-1 Yamaguchi sarcoma viral oncogene homolog 1]; YPEL1 [yippee-like 1 (Drosophila)]; YPEL2 [yippee-like 2 (Drosophila)]; YWHAB [tyrosine 3-monooxygenase/tryptophan 5-monooxygenase activation protein, beta polypeptide]; YWHAQ [tyrosine 3-monooxygenase/tryptophan 5-monooxygenase activation protein, theta polypeptide]; YWHAZ [tyrosine 3-monooxygenase/tryptophan 5-monooxygenase activation protein, zeta polypeptide]; YY1 [YY1 transcription factor]; ZAP70 [zeta-chain (TCR) associated protein kinase 70 kDa]; ZBED1 [zinc finger, BED-type containing 1]; ZC3H12A [zinc finger CCCH-type containing 12A]; ZC3H12D [zinc finger CCCH-type containing 12D]; ZFR [zinc finger RNA binding protein]; ZNF148 [zinc finger protein 148]; ZNF267 [zinc finger protein 267]; ZNF287 [zinc finger protein 287]; ZNF300 [zinc finger protein 300]; ZNF365 [zinc finger protein 365]; ZNF521 [zinc finger protein 521]; ZNF74 [zinc finger protein 74]; and ZPBP2 [zona pellucida binding protein 2].

Examples of proteins associated with Trinucleotide Repeat Disorders include AR (androgen receptor), FMR1 (fragile X mental retardation 1), HTT (huntingtin), DMPK (dystrophia myotonica-protein kinase), FXN (frataxin), ATXN2 (ataxin 2), ATN1 (atrophin 1), FEN1 (flap structure-specific endonuclease 1), TNRC6A (trinucleotide repeat containing 6A), PABPN1 (poly(A) binding protein, nuclear 1), JPH3 (junctophilin 3), MED15 (mediator complex subunit 15), ATXN1 (ataxin 1), ATXN3 (ataxin 3), TBP (TATA box binding protein), CACNA1A (calcium channel, voltage-dependent, P/Q type, alpha 1A subunit), ATXN80S (ATXN8 opposite strand (non-protein coding)), PPP2R2B (protein phosphatase 2, regulatory subunit B, beta), ATXN7 (ataxin 7), TNRC6B (trinucleotide repeat containing 6B), TNRC6C (trinucleotide repeat containing 6C), CELF3 (CUGBP, Elav-like family member 3), MAB21L1 (mab-21-like 1 (C. elegans)), MSH2 (mutS homolog 2, colon cancer, nonpolyposis type 1 (E. coli)), TMEM185A (transmembrane protein 185A), SIX5 (SIX homeobox 5), CNPY3 (canopy 3 homolog (zebrafish)), FRAXE (fragile site, folic acid type, rare, fra(X)(q28) E), GNB2 (guanine nucleotide binding protein (G protein), beta polypeptide 2), RPL14 (ribosomal protein L14), ATXN8 (ataxin 8), INSR (insulin receptor), TTR (transthyretin), EP400 (E1A binding protein p400), GIGYF2 (GRB10 interacting GYF protein 2), OGG1 (8-oxoguanine DNA glycosylase), STC1 (stanniocalcin 1), CNDP1 (carnosine dipeptidase 1 (metallopeptidase M20 family)), C10orf2 (chromosome 10 open reading frame 2), MAML3 mastermind-like 3 (Drosophila), DKC1 (dyskeratosis congenita 1, dyskerin), PAXIP1 (PAX interacting (with transcription-activation domain) protein 1), CASK (calcium/calmodulin-dependent serine protein kinase (MAGUK family)), MAPT (microtubule-associated protein tau), SP1 (Spl transcription factor), POLG (polymerase (DNA directed), gamma), AFF2 (AF4/FMR2 family, member 2), THBS1 (thrombospondin 1), TP53 (tumor protein p53), ESR1 (estrogen receptor 1), CGGBP1 (CGG triplet repeat binding protein 1), ABT1 (activator of basal transcription 1), KLK3 (kallikrein-related peptidase 3), PRNP (prion protein), JUN Gun oncogene), KCNN3 (potassium intermediate/small conductance calcium-activated channel, subfamily N, member 3), BAX (BCL2-associated X protein), FRAXA (fragile site, folic acid type, rare, fra(X)(q27.3) A (macroorchidism, mental retardation)), KBTBD10 (kelch repeat and BTB (POZ) domain containing 10), MBNL1 (muscleblind-like (Drosophila)), RAD51 (RAD51 homolog (RecA homolog, E. coli) (S. cerevisiae)), NCOA3 (nuclear receptor coactivator 3), ERDA1 (expanded repeat domain, CAG/CTG 1), TSC1 (tuberous sclerosis 1), COMP (cartilage oligomeric matrix protein), GCLC (glutamate-cysteine ligase, catalytic subunit), RRAD (Ras-related associated with diabetes), MSH3 (mutS homolog 3 (E. coli)), DRD2 (dopamine receptor D2), CD44 (CD44 molecule (Indian blood group)), CTCF (CCCTC-binding factor (zinc finger protein)), CCND1 (cyclin D1), CLSPN (claspin homolog (Xenopus laevis)), MEF2A (myocyte enhancer factor 2A), PTPRU (protein tyrosine phosphatase, receptor type, U), GAPDH (glyceraldehyde-3-phosphate dehydrogenase), TRTM22 (tripartite motif-containing 22), WT1 (Wilms tumor 1), AHR (aryl hydrocarbon receptor), GPX1 (glutathione peroxidase 1), TPMT (thiopurine S-methyltransferase), NDP (Norrie disease (pseudoglioma)), ARX (aristaless related homeobox), MUS81 (MUS81 endonuclease homolog (S. cerevisiae)), TYR (tyrosinase (oculocutaneous albinism IA)), EGR1 (early growth response 1), UNG (uracil-DNA glycosylase), NUMBL (numb homolog (Drosophila)-like), FABP2 (fatty acid binding protein 2, intestinal), EN2 (engrailed homeobox 2), CRYGC (crystallin, gamma C), SRP14 (signal recognition particle 14 kDa (homologous Alu RNA binding protein)), CRYGB (crystallin, gamma B), PDCD1 (programmed cell death 1), HOXA1 (homeobox A1), ATXN2L (ataxin 2-like), PMS2 (PMS2 postmeiotic segregation increased 2 (S. cerevisiae)), GLA (galactosidase, alpha), CBL (Cas-Br-M (murine) ecotropic retroviral transforming sequence), FTH1 (ferritin, heavy polypeptide 1), IL12RB2 (interleukin 12 receptor, beta 2), OTX2 (orthodenticle homeobox 2), HOXA5 (homeobox AS), POLG2 (polymerase (DNA directed), gamma 2, accessory subunit), DLX2 (distal-less homeobox 2), SIRPA (signal-regulatory protein alpha), OTX1 (orthodenticle homeobox 1), AHRR (aryl-hydrocarbon receptor repressor), MANF (mesencephalic astrocyte-derived neurotrophic factor), TMEM158 (transmembrane protein 158 (gene/pseudogene)), and ENSG00000078687.

Examples of proteins associated with Neurotransmission Disorders include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), HTR2c (5-hydroxytryptamine (serotonin) receptor 2C), SLC1A2 (solute carrier family 1 (glial high affinity glutamate transporter), member 2), GRM5 (glutamate receptor, metabotropic 5), GRM2 (glutamate receptor, metabotropic 2), GABRG3 (gamma-aminobutyric acid (GABA) A receptor, gamma 3), CACNA1B (calcium channel, voltage-dependent, N type, alpha 1B subunit), NOS2 (nitric oxide synthase 2, inducible), SLC6A5 (solute carrier family 6 (neurotransmitter transporter, glycine), member 5), GABRG1 (gamma-aminobutyric acid (GABA) A receptor, gamma 1), NOS3 (nitric oxide synthase 3 (endothelial cell)), GRM3 (glutamate receptor, metabotropic 3), HTR6 (5-hydroxytryptamine (serotonin) receptor 6), SLC1A3 (solute carrier family 1 (glial high affinity glutamate transporter), member 3), GRM7 (glutamate receptor, metabotropic 7), HRH1 (histamine receptor HI), SLC1A1 (solute carrier family 1 (neuronal/epithelial high affinity glutamate transporter, system Xag), member 1), GRM4 (glutamate receptor, metabotropic 4), GLUD2 (glutamate dehydrogenase 2), ADRA2B (adrenergic, alpha-2B-, receptor), SLC1A6 (solute carrier family 1 (high affinity aspartate/glutamate transporter), member 6), GRM6 (glutamate receptor, metabotropic 6), SLC1A7 (solute carrier family 1 (glutamate transporter), member 7), SLC6A11 (solute carrier family 6 (neurotransmitter transporter, GABA), member 11), CACNA1A (calcium channel, voltage-dependent, P/Q type, alpha 1A subunit), CACNA1G (calcium channel, voltage-dependent, T type, alpha 1G subunit), GRM1 (glutamate receptor, metabotropic 1), CACNA1H (calcium channel, voltage-dependent, T type, alpha 1H subunit), GRM8 (glutamate receptor, metabotropic 8), CHRNA3 (cholinergic receptor, nicotinic, alpha 3), P2RY2 (purinergic receptor P2Y, G-protein coupled, 2), TRPV6 (transient receptor potential cation channel, subfamily V, member 6), CACNA 1E (calcium channel, voltage-dependent, R type, alpha 1 E subunit), ACCN1 (amiloride-sensitive cation channel1, neuronal), CACNA1I (calcium channel, voltage-dependent, T type, alpha 11 subunit), GABARAP (GABA (A) receptor-associated protein), P2RY1 (purinergic receptor P2Y, G-protein coupled, 1), P2RY6 (pyrimidinergic receptor P2Y, G-protein coupled, 6), RPH3A (rabphilin 3A homolog (mouse)), HOC (histidine decarboxylase), P2RY14 (purinergic receptor P2Y, G-protein coupled, 14), P2RY4 (pyrimidinergic receptor P2Y, G-protein coupled, 4), P2RY1 0 (purinergic receptor P2Y, G-protein coupled, 10), SLC28A3 (solute carrier family 28 (sodium-coupled nucleoside transporter), member 3), NOSTRIN (nitric oxide synthase trafficker), P2RY13 (purinergic receptor P2Y, G-protein coupled, 13), P2RY8 (purinergic receptor P2Y, G-protein coupled, 8), P2RY11 (purinergic receptor P2Y, G-protein coupled, 11), SLC6A3 (solute carrier family 6 (neurotransmitter transporter, dopamine), member 3), HTR3A (5-hydroxytryptamine (serotonin) receptor 3A), DRD2 (dopamine receptor 02), HTR2A (5-hydroxytryptamine (serotonin) receptor 2A), TH (tyrosine hydroxylase), CNR1 (cannabinoid receptor 1 (brain)), VIP (vasoactive intestinal peptide), NPY (neuropeptide Y), GAL (galaninprepropeptide), TAC1 (tachykinin, precursor 1), SYP (synaptophysin), SLC6A4 (solute carrier family 6 (neurotransmitter transporter, serotonin), member 4), DBH (dopamine beta-hydroxylase (dopamine beta-monooxygenase)), DRD3 (dopamine receptor 03), NR3C1 (nuclear receptor subfamily 3, group C, member 1 (glucocorticoid receptor)), HTR1B (5-hydroxytryptamine (serotonin) receptor IB), GABBR1 (gamma-aminobutyric acid (GABA) B receptor, 1), CALCA (calcitonin-related polypeptide alpha), CRH (corticotropin releasing hormone), HTR1A (5-hydroxytryptamine (serotonin) receptor IA), TACR2 (tachykinin receptor 2), COMT (catechol-0-methyltransferase), GRIN2B (glutamate receptor, ionotropic, N-methyl D-aspartate 2B), GRIN2A (glutamate receptor, ionotropic, N-methyl D-aspartate 2A), PRL (prolactin), ACHE (acetylcholinesterase (Yt blood group)), ADRB2 (adrenergic, beta-2-, receptor, surface), ACE (angiotensin I converting enzyme (peptidyl-dipeptidase A) 1), SNAP25 (synaptosomal-associated protein, 25 kDa), GABRA5 (gamma-aminobutyric acid (GABA) A receptor, alpha 5), MECP2 (methyl CpG binding protein 2 (Rett syndrome)), BCHE (butyrylcholinesterase), ADRBI (adrenergic, beta-1-, receptor), GABRA1 (gamma-aminobutyric acid (GABA) A receptor, alpha 1), GCH1 (GTP cyclohydrolase 1), DOC (dopa decarboxylase (aromatic L-amino acid decarboxylase)), MAOB (monoamine oxidase B), DRD5 (dopamine receptor 05), GABRE (gamma-aminobutyric acid (GABA) A receptor, epsilon), SLC6A2 (solute carrier family 6 (neurotransmitter transporter, noradrenalin), member 2), GABRR2 (gamma-aminobutyric acid (GABA) receptor, rho 2), SV2A (synaptic vesicle glycoprotein 2A), GABRR1 (gamma-aminobutyric acid (GABA) receptor, rho 1), GHRH (growth hormone releasing hormone), CCK (cholecystokinin), PDYN (prodynorphin), SLC6A9 (solute carrier family 6 (neurotransmitter transporter, glycine), member 9), KCND1 (potassium voltage-gated channel, Shal-related subfamily, member 1), SRR (serine racemase), DYT1 0 (dystonia 10), MAPT (microtubule-associated protein tau), APP (amyloid beta (A4) precursor protein), CTSB (cathepsin B), ADA (adenosine deaminase), AKT1 (v-akt murine thymoma viral oncogene homolog 1), GR1N1 (glutamate receptor, ionotropic, N-methyl D-aspartate 1), BDNF (brain-derived neurotrophic factor), HMOX1 (heme oxygenase (decycling) 1), OPRM1 (opioid receptor, mu 1), GRTN2C (glutamate receptor, ionotropic, N-methyl D-aspartate 2C), GRIA1 (glutamate receptor, ionotropic, AMPA 1), GABRA6 (gamma-aminobutyric acid (GABA) A receptor, alpha 6), FOS (FBJ murine osteosarcoma viral oncogene homolog), GABRG2 (gamma-aminobutyric acid (GABA) A receptor, gamma 2), GABRB3 (gamma-aminobutyric acid (GABA) A receptor, beta 3), OPRK1 (opioid receptor, kappa 1), GABRB2 (gamma-aminobutyric acid (GABA) A receptor, beta 2), GABRD (gamma-aminobutyric acid (GABA) A receptor, delta), ALDH5A1 (aldehyde dehydrogenase 5 family, member A1), GAD1 (glutamate decarboxylase 1 (brain, 67 kDa)), NSF (N-ethylmaleimide-sensitive factor), GRIN2D (glutamate receptor, ionotropic, N-methyl D-aspartate 2D), ADORA1 (adenosine A1 receptor), GABRA2 (gamma-aminobutyric acid (GABA) A receptor, alpha 2), GLRA1 (glycine receptor, alpha 1), CHRM3 (cholinergic receptor, muscarinic 3), CHAT (choline acetyltransferase), KNG1 (kininogen 1), HMOX2 (heme oxygenase (decycling) 2), DRD4 (dopamine receptor D4), MAOA (monoamine oxidase A), CHRM2 (cholinergic receptor, muscarinic 2), ADORA2A (adenosine A2a receptor), STXBP1 (syntaxin binding protein 1), GABRA3 (gamma-aminobutyric acid (GABA) A receptor, alpha 3), TPH1 (tryptophan hydroxylase 1), HCRTR1 (hypocretin (orexin) receptor 1), HCRTR2 (hypocretin (orexin) receptor 2), CHRMI (cholinergic receptor, muscarinic 1), FOLHI (folate hydrolase (prostate-specific membrane antigen) 1), AANAT (arylalkylamine N-acetyltransferase), INS (insulin), NR3C2 (nuclear receptor subfamily 3, group C, member 2), FAAH (fatty acid amide hydrolase), GALR2 (galanin receptor 2), ADCYAP1 (adenylate cyclase activating polypeptide 1 (pituitary)), PPP1R1B (protein phosphatase 1, regulatory (inhibitor) subunit IB), HOMER1 (homer homolog 1 (Drosophila)), ADCY10O (adenylate cyclase 10 (soluble)), PSEN2 (presenilin 2 (Alzheimer disease 4)), UBE3A (ubiquitin protein ligase E3A), SOD1 (superoxide dismutase 1, soluble), LYN (v-yes-1 Yamaguchi sarcoma viral related oncogene homolog), TSC2 (tuberous sclerosis 2), PRKCA (protein kinase C, alpha), PPARG (peroxisome proliferator-activated receptor gamma), ESR1 (estrogen receptor 1), NTRK1 (neurotrophic tyrosine kinase, receptor, type 1), EGFR (epidermal growth factor receptor (erythroblastic leukemia viral (v-erb-b) oncogene homolog, avian)), S100B (S100 calcium binding protein B), NTRK3 (neurotrophic tyrosine kinase, receptor, type 3), PLCG2 (phospholipase C, gamma 2 (phosphatidylinositol-specific)), NTRK2 (neurotrophic tyrosine kinase, receptor, type 2), DNMT1 (DNA (cytosine-5-)-methyltransferase 1), EGF (epidermal gro ih factor (beta-urogastrone)), GRIA3 (glutamate receptor, ionotrophic, AMPA 3), NCAM1 (neural cell adhesion molecule 1), CDKN1A (cyclin-dependent kinase inhibitor 1A (p21, Cip1)), BCL2L1 (BCL2-like 1), TP53 (tumor protein p53), CASP9 (caspase 9, apoptosis-related cysteine peptidase), CCKBR (cholecystokinin B receptor), PARK2 (Parkinson's disease (autosomal recessive, juvenile) 2, parkin), ADRA1B (adrenergic, alpha-1B-, receptor), CASP3 (caspase 3, apoptosis-related cysteine peptidase), PRNP (prion protein), CRHR1 (corticotropin releasing hormone receptor 1), L1CAM (L1 cell adhesion molecule), NGFR (nerve growth factor receptor (TNFR superfamily, member 16)), CREB1 (cAMP responsive element binding protein 1), PLCG1 (phospholipase C, gamma 1), CAV1 (caveolin 1, caveolae protein, 22 kDa), ABCC8 (ATP-binding cassette, sub-family C(CFTR/MRP), member 8), ACTN2 (actinin, alpha 2), GR1A2 (glutamate receptor, ionotropic, AMPA 2), HPRT1 (hypoxanthine phosphoribosyltransferase 1), SYN1 (synapsin T), CSNK2A1 (casein kinase 2, alpha 1 polypeptide), GRIK1 (glutamate receptor, ionotropic, kainate 1), ABCB1 (ATP-binding cassette, sub-family B (MDR/TAP), member 1), AVPR2 (arginine vasopressin receptor 2), HTR4 (5-hydroxytryptamine (serotonin) receptor 4), C3 (complement component 3), AGT (angiotensinogen (serpin peptidase inhibitor, clade A, member 8)), AGTR1 (angiotensin II receptor, type 1), CDK5 (cyclin-dependent kinase 5), LRP1 (low density lipoprotein receptor-related protein 1), ARRB2 (arrestin, beta 2), PLD2 (phospholipase D2), OPRD1 (opioid receptor, delta 1), GNB3 (guanine nucleotide binding protein (G protein), beta polypeptide 3), PIK3CG (phosphoinositide-3-kinase, catalytic, gamma polypeptide), APAF1 (apoptotic peptidase activating factor 1), SSTR2 (somatostatin receptor 2), IL2 (interleukin 2), ADORA3 (adenosine A3 receptor), ADRA1A (adrenergic, alpha-1A-, receptor), HTR7 (5-hydroxytryptamine (serotonin) receptor 7 (adenylate cyclase-coupled)), ADRBK2 (adrenergic, beta, receptor kinase 2), ALOX5 (arachidonate 5-lipoxygenase), NPR1 (natriuretic peptide receptor A/guanylate cyclase A (atrionatriuretic peptide receptor A)), AVPR1A (arginine vasopressin receptor 1A), CHRNB1 (cholinergic receptor, nicotinic, beta 1 (muscle)), SET (SET nuclear oncogene), PAH (phenylalanine hydroxylase), POMC (proopiomelanocortin), LEPR (leptin receptor), SDC2 (syndecan2), VIPR1 (vasoactive intestinal peptide receptor 1), DBI (diazepam binding inhibitor (GABA receptor modulator, acyl-Coenzyme A binding protein)), NPY1R (neuropeptide Y receptor Y1), NPR2 (natriuretic peptide receptor B/guanylate cyclase B (atrionatriuretic peptide receptor B)), CNR2 (cannabinoid receptor 2 (macrophage)), LEP (leptin), CCKAR (cholecystokinin A receptor), GLRB (glycine receptor, beta), KCNQ2 (potassium voltage-gated channel, KQT-like subfamily, member 2), CHRNA2 (cholinergic receptor, nicotinic, alpha 2 (neuronal)), BDKRB2 (bradykinin receptor B2), CHRNA1 (cholinergic receptor, nicotinic, alpha 1 (muscle)), CHRND (cholinergic receptor, nicotinic, delta), CHRNA7 (cholinergic receptor, nicotinic, alpha 7), PLD1 (phospholipase D1, phosphatidylcholine-specific), NRXN1 (neurexin 1), NRP1 (neuropilin 1), DLG3 (discs, large homolog 3 (Drosophila)), GNAQ (guanine nucleotide binding protein (G protein), q polypeptide), DRD1 (dopamine receptor D1), PRKG1 (protein kinase, cGMP-dependent, type I), CNTNAP2 (contactin associated protein-like 2), EDN3 (endothelin3), ABAT (4-aminobutyrate aminotransferase), TD02 (tryptophan2,3-dioxygenase), NEUROD1 (neurogenic differentiation 1), CHRNE (cholinergic receptor, nicotinic, epsilon), CHRNB2 (cholinergic receptor, nicotinic, beta 2 (neuronal)), CHRNB3 (cholinergic receptor, nicotinic, beta 3), HTR1D (5-hydroxytryptamine (serotonin) receptor ID), ADRA1D (adrenergic, alpha-1D-, receptor), HTR2B (5-hydroxytryptamine (serotonin) receptor 2B), GRIK3 (glutamate receptor, ionotropic, kainate 3), NPY2R (neuropeptide Y receptor Y2), GRIK5 (glutamate receptor, ionotropic, kainate 5), GRIA4 (glutamate receptor, ionotrophic, AMPA 4), EDN1 (endothelin 1), PRLR (prolactin receptor), GABRB1 (gamma-aminobutyric acid (GABA) A receptor, beta 1), GARS (glycyl-tRNA synthetase), GRIK2 (glutamatereceptor, ionotropic, kainate 2), ALOX12 (arachidonate 12-lipoxygenase), GAD2 (glutamate decarboxylase 2 (pancreatic islets and brain, 65 kDa)), LHCGR (luteinizing hormone/choriogonadotropin receptor), SHMT1 (serine hydroxymethyltransferase 1 (soluble)), PDXK (pyridoxal (pyridoxine, vitamin B6) kinase), L1F (leukemia inhibitory factor (cholinergic differentiation factor)), PLCD1 (phospholipase C, delta 1), NTF3 (neurotrophin 3), NFE2L2 (nuclear factor (erythroid-derived 2)-like 2), PLCB4 (phospholipase C, beta 4), GNRHR (gonadotropin-releasing hormone receptor), NLGN1 (neuroligin 1), PPP2R4 (protein phosphatase 2A activator, regulatory subunit 4), SSTR3 (somatostatin receptor 3), CRHR2 (corticotropin releasing hormone receptor 2), NGF (nerve growth factor (beta polypeptide)), NRCAM (neuronal cell adhesion molecule), NRXN3 (neurexin 3), GNRH1 (gonadotropin-releasing hormone 1 (luteinizing-releasing hormone)), TRHR (thyrotropin-releasing hormone receptor), ARRB1 (arrestin, beta 1), INPP1 (inositol polyphosphate-1-phosphatase), PTN (pleiotrophin), PSMD10 (proteasome (prosome, macropain) 26S subunit, non-ATPase, 10), DLG1 (discs, large homolog 1 (Drosophila)), PSMB8 (proteasome (prosome, macropain) subunit, beta type, 8 (large multifunctional peptidase 7)), CYCS (cytochrome c, somatic), ADORA2B (adenosine A2b receptor), ADRB3 (adrenergic, beta-3-, receptor), CHGA (chromogranin A (parathyroid secretory protein 1)), ADM (adrenomedullin), GABRP (gamma-aminobutyric acid (GABA) A receptor, pi), GLRA2 (glycine receptor, alpha 2), PRKG2 (protein kinase, cGMP-dependent, type II), GLS (glutaminase), TACR3 (tachykinin receptor 3), ALDH7A1 (aldehyde dehydrogenase 7 family, member A1), GABBR2 (gamma-aminobutyric acid (GABA) B receptor, 2), GDNF (glial cell derived neurotrophic factor), CNTFR (ciliary neurotrophic factor receptor), CNTN2 (contactin 2 (axonal)), TOR1A (torsin family 1, member A (torsin A)), CNTN1 (contactin 1), CAMK1 (calcium/calmodulin-dependent protein kinase I), NPPB (natriuretic peptide precursor B), OXTR (oxytocin receptor), OSM (oncostatin M), VIPR2 (vasoactive intestinal peptide receptor 2), CHRNB4 (cholinergic receptor, nicotinic, beta 4), CHRNA5 (cholinergic receptor, nicotinic, alpha 5), AVP (arginine vasopressin), RELN (reelin), GRLF1 (glucocorticoid receptor DNA binding factor 1), NPR3 (natriuretic peptide receptor C/guanylate cyclase C (atrionatriuretic peptide receptor C)), GRIK4 (glutamate receptor, ionotropic, kainate 4), KISS1 (KiSS-1metastasis-suppressor), HTR5A (5-hydroxytryptamine (serotonin) receptor 5A), ADCYAP1R1 (adenylate cyclase activating polypeptide 1 (pituitary) receptor type I), GABRA4 (gall1111a-aminobutyric acid (GABA) A receptor, alpha 4), GLRA3 (glycine receptor, alpha 3), INHBA (inhibin, beta A), DLG2 (discs, large homolog 2 (Drosophila)), PPYR1 (pancreatic polypeptide receptor 1), SSTR4 (somatostatin receptor 4), NPPA (natriuretic peptide precursor A), SNAP23 (synaptosomal-associated protein, 23 kDa), AKAP9 (A kinase (PRKA) anchor protein (yotiao) 9), NRXN2 (neurexin 2), FHL2 (four and a half LIM domains 2), TJPI (tight junction protein 1 (zona occludens 1)), NRGI (neuregulin 1), CAMK4 (calcium/calmodulin-dependent protein kinase IV), CAV3 (caveolin 3), VAMP2 (vesicle-associated membrane protein 2 (synaptobrevin 2)), GALRI (galanin receptor 1), GHRHR (growth hormone releasing hormone receptor), HTRIE (5-hydroxytryptamine (serotonin) receptor IE), PENK (proenkephalin), HTT (huntingtin), HOXAI (homeobox AI), NPY5R (neuropeptide Y receptor Y5), UNC119 (unc-119 homolog (C. elegans)), TAT (tyrosine aminotransferase), CNTF (ciliary neurotrophic factor), SHMT2 (serine hydroxymethyltransferase 2 (mitochondrial)), ENTPDI (ectonucleoside triphosphate diphosphohydrolase 1), GRIP I (glutamate receptor interacting protein 1), GRP (gastrin-releasing peptide), NCAM2 (neural cell adhesion molecule 2), SSTRI (somatostatin receptor 1), CLTB (clathrin, light chain (Lcb)), DAO (D-amino-acid oxidase), QDPR (quinoid dihydropteridine reductase), PYY (peptide YY), PNMT (phenylethanolamine N-methyltransferase), NTSRI (neurotensin receptor 1 (high affinity)), NTS (neurotensin), HCRT (hypocretin (orexin) neuropeptide precursor), SNAP29 (synaptosomal-associated protein, 29 kDa), SNAP91 (synaptosomal-associated protein, 91 kDa homolog (mouse)), MADD (MAP-kinase activating death domain), IDO1 (indoleamine 2,3-dioxygenase 1), TPH2 (tryptophan hydroxylase 2), TAC3 (tachykinin 3), GRTN3A (glutamate receptor, ionotropic, N-methyl-D-aspartate 3A), REN (renin), GALR3 (galanin receptor 3), MAGI2 (membrane associated guanylate kinase, WW and PDZ domain containing 2), KCNJ9 (potassium inwardly-rectifying channel, subfamily J, member 9), BDKRBI (bradykinin receptor B1), CHRNA6 (cholinergic receptor, nicotinic, alpha 6), CHRM5 (cholinergic receptor, muscarinic 5), CHRNG (cholinergic receptor, nicotinic, gamma), SLC6A1 (solute carrier family 6 (neurotransmitter transporter, GABA), member 1), ENTPD2 (ectonucleoside triphosphate diphosphohydrolase 2), CALCB (calcitonin-related polypeptide beta), SHBG (sex hormone-binding globulin), SERPINA6 (scrpin peptidase inhibitor, clade A (alpha-I antiprotcinase, antitrypsin), member 6), NRG2 (neuregulin 2), PNOC (prepronociceptin), NAPA (N-ethylmaleimide-sensitive factor attachment protein, alpha), PICK I (protein interacting with PRKCA 1), PLCD4 (phospholipase C, delta 4), GCDH (glutaryl-Coenzyme A dehydrogenase), NLGN2 (neuroligin 2), NBEA (neurobeachin), ATPIOA (ATPase, class V, type 10A), RAPGEF4 (Rap guanine nucleotide exchange factor (GEF) 4), UCN (urocortin), PCSK6 (proprotein convertase subtilisin/kexin type 6), HTRIF (5-hydroxytryptamine (serotonin) receptor IF), SGCB (sarcoglycan, beta (43 kDa dystrophin-associated glycoprotein)), GABRQ (gamma-aminobutyric acid (GABA) receptor, theta), GHRL (ghrelin/obestatin prepropeptide), NCALD (neurocalcin delta), NEUROD2 (neurogenic differentiation 2), DPEPI (dipeptidase 1 (renal)), SLCIA4 (solute carrier family 1 (glutamate/neutral amino acid transporter), member 4), DNM3 (dynamin 3), SLC6A12 (solute carrier family 6 (neurotransmitter transporter, betaine/GABA), member 12), SLC6A6 (solute carrier family 6 (neurotransmitter transporter, taurine), member 6), YMEILI (YMEI-like 1 (S. cerevisiae)), VSNLI (visinin-like 1), SLC17A7 (solute carrier family 17 (sodium-dependent inorganic phosphate cotransporter), member 7), HOMER2 (homer homolog 2 (Drosophila)), SYT7 (synaptotagmin VII), TFIP11 (tuftelin interacting protein 11), GMFB (glia maturation factor, beta), PREB (prolactin regulatory element binding), NTSR2 (neurotensin receptor 2), NTF4 (neurotrophin 4), PPP1R9B (protein phosphatase 1, regulatory (inhibitor) subunit 9B), DISCI (dismpted in schizophrenia 1), NRG3 (neuregulin 3), OXT (oxytocin, prepropeptide), TRH (thyrotropin-releasing hormone), NISCH (nischarin), CRHBP (corticotropin releasing hormone binding protein), SLC6A13 (solute carrier family 6 (neurotransmitter transporter, GABA), member 13), NPPC (natriuretic peptide precursor C), CNTN3 (contactin 3 (plasmacytoma associated)), KAT5 (K (lysine) acetyltransferase 5), CNTN6 (contactin 6), KIAA0101 (KIAA0101), PANX1 (pannexin 1), CTSL1 (cathepsin L), EARS2 (glutamyl-tRNA synthetase 2, mitochondrial (putative)), CRIPT (cysteine-rich PDZ-binding protein), CORT (cortistatin), DLGAP4 (discs, large (Drosophila) homolog-associated protein 4), ASTN2 (astrotactin 2), HTR3B (5-hydroxytryptamine (serotonin) receptor 3B), PMCH (pro-melanin-concentrating hormone), TSPO (translocator protein (18 kDa)), GDF2 (growth differentiation factor 2), CNTNAP1 (contactin associated protein 1), GNRH2 (gonadotropin-releasing hormone 2), AUTS2 (autism susceptibility candidate 2), SV2C (synaptic vesicle glycoprotein 2C), CARTPT (CART prepropeptide), NSUN4 (NOP2/Sun domain family, member 4), CNTN5 (contactin 5), NEUROD4 (neurogenic differentiation 4), NEUROG1 (neurogenin 1), SLTM (SAFB-like, transcription modulator), GNRHR2 (gonadotropin-releasing hormone (type 2) receptor 2), ASTN1 (astrotactin 1), SLC22A18 (solute carrier family 22, member 18), SLC17A6 (solute carrier family 17 (sodium-dependent inorganic phosphate cotransporter), member 6), GABRR3 (gamma-aminobutyric acid (GABA) receptor, rho 3), DAOA (D-amino acid oxidase activator), ENSG00000123384, nd NOS2P1 (nitric oxide synthase 2 pseudogene 1).

Examples of neurodevelopmental-associated sequences include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], ABCA13 [ATP-binding cassette, sub-family A (ABC1), member 13], ABCA2 [ATP-binding cassette, sub-family A (ABC1), member 2], ABCB1 [ATP-binding cassette, sub-family B (MDR/TAP), member 1], ABCB11 [ATP-binding cassette, sub-family B (MDR/TAP), member 11], ABCB4 [ATP-binding cassette, sub-family B (MDR/TAP), member 4], ABCB6 [ATP-binding cassette, sub-family B (MDR/TAP), member 6], ABCB7 [ATP-binding cassette, sub-family B (MDR/TAP), member 7], ABCC1 [ATP-binding cassette, sub-family C(CFTR/MRP), member 1], ABCC2 [ATP-binding cassette, sub-family C (CFTR/MRP), member 2], ABCC3 [ATP-binding cassette, sub-family C (CFTR/MRP), member 3], ABCC4 [ATP-binding cassette, sub-family C (CFTR/MRP), member 4], ABCD1 [ATP-binding cassette, sub-family D (ALD), member 1], ABCD3 [ATP-binding cassette, sub-family D (ALD), member 3], ABCG1 [ATP-binding cassette, sub-family G (WHITE), member 1], ABCC2 [ATP-binding cassette, sub-family G (WHITE), member 2], ABCC4 [ATP-binding cassette, sub-family G (WHITE), member 4], ABHD11 [abhydrolase domain containing 11], ABil [abl-interactor 1], ABL1 [c-abl oncogene 1, receptor tyrosine kinase], ABL2 [v-abl Abelson murine leukemia viral oncogene homolog 2 (arg, Abelson-related gene)], ABLIM1 [actin binding LIM protein 1], ABLIM2 [actin binding LIM protein family, member 2], ABLIM3 [actin binding LIM protein family, member 3], ABO [ABO blood group (transferase A, alpha 1-3-N-acetylgalactosaminyltransferase; transferase B, alpha 1-3-galactosyltransferase)], ACAA1 [acetyl-Coenzyme A acyltransferase 1], ACACA [acetyl-Coenzyme A carboxylase alpha], ACACB [acetyl-Coenzyme A carboxylase beta], ACADL [acyl-Coenzyme A dehydrogenase, long chain], ACADM [acyl-Coenzyme A dehydrogenase, C-4 to C-12 straight chain], ACADS [acyl-Coenzyme A dehydrogenase, C-2 to C-3 short chain], ACADSB [acyl-Coenzyme A dehydrogenase, short/branched chain], ACAN [aggrecan], ACAT2 [acetyl-Coenzyme A acetyltransferase 2], ACCN1 [amiloride-sensitive cation channel I, neuronal], ACE [angiotensin I converting enzyme (peptidyl-dipeptidase A) 1], ACE2 [angiotensin I converting enzyme (peptidyl-dipeptidase A) 2], ACHE [acetylcholinesterase (Yt blood group)], ACLY [ATP citrate lyase], ACO1 [aconitase 1, soluble], ACTAI [actin, alpha 1, skeletal muscle], ACTB [actin, beta], ACTC1 [actin, alpha, cardiac muscle 1], ACTG1 [actin, gamma 1], ACTL6A [actin-like 6A], ACTL6B [actin-like 6B], ACTN1 [actinin, alpha 1], ACTR1A [ARP1 actin-related protein 1 homolog A, centractin alpha (yeast)], ACTR2 [ARP2 actin-related protein 2 homolog (yeast)], ACTR3 [ARP3 actin-related protein 3 homolog (yeast)], ACTR3B [ARP3 actin-related protein 3 homolog B (yeast)], ACVR1 [activin A receptor, type I], ACVR2A [activin A receptor, type IIA], ADA [adenosine deaminase], ADAM1O [ADAM metallopeptidase domain 10], ADAMI I [ADAM metallopeptidase domain 11], ADAM12 [ADAM metallopeptidase domain 12], ADAM15 [ADAM metallopeptidase domain 15], ADAM17 [ADAM metallopeptidase domain 17], ADAM18 [ADAM metallopeptidase domain 18], ADAM19 [ADAM metallopeptidase domain 19 (meltrin beta)], ADAM2 [ADAM metallopeptidase domain 2], ADAM20 [ADAM metallopeptidase domain 20], ADAM21 [ADAM metallopeptidase domain 21], ADAM22 [ADAM metallopeptidase domain 22], ADAM23 [ADAM metallopeptidase domain 23], ADAM28 [ADAM metallopeptidase domain 28], ADAM29 [ADAM metallopeptidase domain 29], ADAM30 [ADAM metallopeptidase domain 30], ADAM8 [ADAM metallopeptidase domain 8], ADAMS [ADAM metallopeptidase domain 9 (meltrin gamma)], ADAMTS1 [ADAM metallopeptidase with thrombospondin type 1 motif, 1], ADAMTS13 [ADAM metallopeptidase with thrombospondin type 1 motif, 13], ADAMTS4 [ADAM metallopeptidase with thrombospondin type 1 motif, 4], ADAMTS5 [ADAM metallopeptidase with thrombospondin type 1 motif, 5], ADAP2 [ArfGAP with dual PH domains 2], ADAR [adenosine deaminase, RNA-specific], ADARB1 [adenosine deaminase, RNA-specific, B1 (RED1 homolog rat)], ADCY1 [adenylate cyclase 1 (brain)], ADCY10 [adenylate cyclase 10 (soluble)], ADCYAP1 [adenylate cyclase activating polypeptide 1 (pituitary)], ADD1 [adducin 1 (alpha)], ADD2 [adducin 2 (beta)], ADRIA [alcohol dehydrogenase 1A (class I), alpha polypeptide], ADIPOQ [adiponectin, C1Q and collagen domain containing], ADK [adenosine kinase], ADM [adrenomedullin], ADNP [activity-dependent neuroprotector homeobox], ADORA1 [adenosine A1 receptor], ADORA2A [adenosine A2a receptor], ADORA2B [adenosine A2b receptor], ADORA3 [adenosine A3 receptor], ADRA1B [adrenergic, alpha-1B-, receptor], ADRA2A [adrenergic, alpha-2A-, receptor], ADRA2B [adrenergic, alpha-2B-, receptor], ADRA2C [adrenergic, alpha-2C-, receptor], ADRB1 [adrenergic, beta-1-, receptor], ADRB2 [adrenergic, beta-2-, receptor, surface], ADRB3 [adrenergic, beta-3-, receptor], ADRBK2 [adrenergic, beta, receptor kinase 2], ADSL [adenylosuccinate lyase], AFF2 [AF4/FMR2 family, member 2], AFM [afamin], AFP [alpha-fetoprotein], AGAPI [ArfGAP with GTPase domain, ankyrin repeat and PH domain I], AGER [advanced glycosylation end product-specific receptor], AGFG1 [ArfGAP with FG repeats 1], AGPS [alkylglycerone phosphate synthase], AGRN [agrin], AGRP [agouti related protein homolog (mouse)], AGT [angiotensinogen (serpin peptidase inhibitor, clade A, member 8)], AGTR1 [angiotensin II receptor, type I], AGTR2 [angiotensin II receptor, type 2], AHOY [adenosylhomocysteinase], AHi1 [Abelson helper integration site I], AHR [aryl hydrocarbon receptor], AHSG [alpha-2-HS-glycoprotein], AICDA [activation-induced cytidine deaminase], AIFMI [apoptosis-inducing factor, mitochondrion-associated, 1], AIRE [autoimmune regulator], AKAP I2 [A kinase (PRKA) anchor protein I2], AKAP9 [A kinase (PRKA) anchor protein (yotiao) 9], AKRIAI [aldo-keto reductase family I, member AI (aldehyde reductase)], AKR 1B1 [aldo-keto reductase family 1, member B1 (aldose reductase)], AKR 1 C3 [aldo-keto reductase family I, member C3 (3-alpha hydroxysteroid dehydrogenase, type II)], AKT1 [v-akt murine thymoma viral oncogene homolog I], AKT2 [v-akt murine thymoma viral oncogene homolog 2], AKT3 [v-akt murine thymoma viral oncogene homolog 3 (protein kinase B, gamma)], ALAD [aminolevulinate, delta-, dehydratase], ALB [albumin], ALB [albumin], ALCAM [activated leukocyte cell adhesion molecule], ALDH1 A1 [aldehyde dehydrogenase 1 family, member A 1], ALDH3A 1 [aldehyde dehydrogenase 3 family, memberAI], ALDH5AI [aldehyde dehydrogenase 5 family, member AI], ALDH7AI [aldehyde dehydrogenase 7 family, member AI], ALDH9AI [aldehyde dehydrogenase 9 family, member AI], ALDOA [aldolase A, fructose-bisphosphate], ALDOB [aldolase B, fructose-bisphosphate], ALDOC [aldolase C, fructose-bisphosphate], ALK [anaplastic lymphoma receptor tyrosine kinase], ALOXI2 [arachidonate I2-lipoxygenase], ALOX5 [arachidonate 5-lipoxygenase], ALOX5AP [arachidonate 5-lipoxygenase-activating protein], ALPI [alkaline phosphatase, intestinal], ALPL [alkaline phosphatase, liver/bone/kidney], ALPP [alkaline phosphatase, placental (Regan isozyme)], ALS2 [amyotrophic lateral sclerosis 2 Guvenile)], AMACR [alpha-methylacyl-CoA racemase], AMBP [alpha-I-microglobulin!bikunin precursor], AMPH [amphiphysin], ANG [angiogenin, ribonuclease, RNase A family, 5], ANGPTI [angiopoietin 1], ANGPT2 [angiopoietin 2], ANGPTL3 [angiopoietin-like 3], ANKI [ankyrin I, erythrocytic], ANK3 [ankyrin 3, node of Ranvier (ankyrin G)], ANKRDI [ankyrin repeat domain I (cardiac muscle)], ANP32E [acidic (leucine-rich) nuclear phosphoprotein 32 family, member E], ANPEP [alanyl (membrane) aminopeptidase], ANXAI [annexin AI], ANXA2 [annexin A2], ANXA5 [annexin AS], API S I [adaptor-related protein complex I, sigma I subunit], API S2 [adaptor-related protein complex I, sigma 2 subunit], AP2AI [adaptor-related protein complex 2, alpha I subunit], AP2B1 [adaptor-related protein complex 2, beta 1 subunit], APAF1 [apoptotic peptidase activating factor I], APBAI [amyloid beta (A4) precursor protein-binding, family A, member I], APBA2 [amyloid beta (A4) precursor protein-binding, family A, member 2], APBBI [amyloid beta (A4) precursor protein-binding, family B, member I (Fe65)], APBB2 [amyloid beta (A4) precursor protein-binding, family B, member 2], APC [adenomatous polyposis coli], APCS [amyloid P component, serum], APEX1 [APEX nuclease (multifunctional DNA repair enzyme) 1], APH1B [anterior pharynx defective I homolog B (C. elegans)], APLPI [amyloid beta (A4) precursor-like protein I], APOA1 [apolipoprotein A-I], APOA5 [apolipoprotein A-V], APOB [apolipoprotein B (including Ag(x) antigen)], APOC2 [apolipoprotein C-II], APOD [apolipoprotein D], APOE [apolipoprotein E], APOM [apolipoprotein M], APP [amyloid beta (A4) precursor protein], APPL1 [adaptor protein, phosphotyrosine interaction, PH domain and leucine zipper containing 1], APRT [adenine phosphoribosyltransferase], APTX [aprataxin], AQP1 [aquaporin 1 (Colton blood group)], AQP2 [aquaporin 2 (collecting duct)], AQP3 [aquaporin 3 (Gill blood group)], AQP4 [aquaporin 4], AR [androgen receptor], ARC [activity-regulated cytoskeleton-associated protein], AREG [amphiregulin], ARFGEF2 [ADP-ribosylation factor guanine nucleotide-exchange factor 2 (brefeldin A-inhibited)], ARG1 [arginase, liver], ARHGAP1 [Rho GTPase activating protein 1], ARHGAP32 [Rho GTPase activating protein 32], ARHGAP4 [Rho GTPase activating protein 4], ARHGAP5 [Rho GTPase activating protein 5], ARHGDTA [Rho GDP dissociation inhibitor (GDT) alpha], ARHGEF1 [Rho guanine nucleotide exchange factor (GEF) 1], ARHGEF10 [Rho guanine nucleotide exchange factor (GEF) 10], ARHGEF11 [Rho guanine nucleotide exchange factor (GEF) 11], ARHGEF12 [Rho guanine nucleotide exchange factor (GEF) 12], ARHGEF15 [Rho guanine nucleotide exchange factor (GEF) 15], ARHGEF16 [Rho guanine nucleotide exchange factor (GEF) 16], ARHGEF2 [Rho/Rae guanine nucleotide exchange factor (GEF) 2], ARHGEF3 [Rho guanine nucleotide exchange factor (GEF) 3], ARHGEF4 [Rho guanine nucleotide exchange factor (GEF) 4], ARHGEF5 [Rho guanine nucleotide exchange factor (GEF) 5], ARHGEF6 [Rac/Cdc42 guanine nucleotide exchange factor (GEF) 6], ARHGEF7 [Rho guanine nucleotide exchange factor (GEF) 7], ARHGEF9 [Cdc42 guanine nucleotide exchange factor (GEF) 9], ARID1A [AT rich interactive domain 1A (SWI-like)], ARID1B [AT rich interactive domain 1B (SWil-like)], ARL13B [ADP-ribosylation factor-like 13B], ARPC1A [actin related protein 2/3 complex, subunit 1A, 41 kDa], ARPC1B [actin related protein 2/3 complex, subunit IB, 41 kDa], ARPC2 [actin related protein 2/3 complex, subunit 2, 34 kDa], ARPC3 [actin related protein 2/3 complex, subunit 3, 21 kDa], ARPC4 [actin related protein 2/3 complex, subunit 4, 20 kDa], ARPC5 [actin related protein 2/3 complex, subunit 5, 16 kDa], ARPC5L [actin related protein 2/3 complex, subunit 5-like], ARPP19 [cAMP-regulated phosphoprotein, 19 kDa], ARR3 [arrestin 3, retinal (X-arrestin)], ARRB2 [arrestin, beta 2], ARSA [arylsulfatase A], ARTN [artemin], ARX [aristaless related homeobox], ASCL1 [achaete-scute complex homolog 1 (Drosophila)], ASMT [acetylserotonin 0-methyltransferase], ASPA [aspartoacylase (Canavan disease)], ASPG [asparaginase homolog (S. cerevisiae)], ASPH [aspartate beta-hydroxylase], ASPM [asp (abnormal spindle) homolog, microcephaly associated (Drosophila)], ASRGL1 [asparaginase like 1], ASS1 [argininosuccinate synthase 1], ASTNI [astrotactin 1], ATAD5 [ATPase family, AAA domain containing 5], ATF2 [activating transcription factor 2], ATF4 [activating transcription factor 4 (tax-responsive enhancer element B67)], ATF6 [activating transcription factor 6], ATM [ataxia telangiectasia mutated], ATOHI [atonal homolog 1 (Drosophila)], ATOXI [ATXI antioxidant protein 1 homolog (yeast)], ATPIOA [ATPase, class V, type 10A], ATP2A2 [ATPase, Ca++ transporting, cardiac muscle, slow twitch 2], ATP2B2 [ATPase, Ca++ transporting, plasma membrane 2], ATP2B4 [ATPase, Ca++ transporting, plasma membrane 4], ATP50 [ATP synthase, H+ transporting, mitochondrial F1 complex, 0 subunit], ATP6AP1 [ATPase, H+ transporting, lysosomal accessmy protein 1], ATP6VOC [ATPase, R+ transporting, lysosomall6 kDa, VO subunit c], ATP7A [ATPase, Cu++ transpmiing, alpha polypeptide], ATPSA1 [ATPase, aminophospholipid transpmier (APLT), class I, type SA, member 1], ATR [ataxia telangiectasia and Rad3 related], ATRN [attractin], ATRX [alpha thalassemia/mental retardation syndrome X-linked (RAD54 homolog, S. cerevisiae)], ATXN1 [ataxin 1], ATXN2 [ataxin 2], ATXN3 [ataxin 3], AURKA [aurora kinase A], AUTS2 [autism susceptibility candidate 2], AVP [arginine vasopressin], AVPR1A [arginine vasopressin receptor 1A], AXIN2 [axin 2], AXL [AXL receptor tyrosine kinase], AZU1 [azurocidin 1], B2M [beta-2-microglobulin], B3GNT2 [UDP-G1cNAc:betaGa1 beta-1 [3-N-acetylglucosaminyltransferase 2], B9D1 [B9 protein domain 1], BACE1 [beta-site APP-cleaving enzyme 1], BACE2 [beta-site APP-cleaving enzyme 2], BACH I [BTB and CNC homology 1, basic leucine zipper transcription factor 1], BAD [BCL2-associated agonist of cell death], BACE2 [B melanoma antigen family, member 2], BAIAP2 [BAil-associated protein 2], BAIAP2L1 [BAil-associated protein 2-like 1], BAK1 [BCL2-antagonist/killer 1], BARD I [BRCA1 associated RING domain 1], BARRL1 [BarR-like homeobox 1], BARHL2 [BarR-like homeobox 2], BASP1 [brain abundant, membrane attached signal protein 1], BAX [BCL2-associated X protein], BAZ1A [bromodomain adjacent to zinc finger domain, 1 A], BAZ1 B [bromodomain adjacent to zinc finger domain, 1 B], BBS9 [Bardet-Biedl syndrome 9], BCAR 1 [breast cancer anti-estrogen resistance 1], BCRE [butyrylcholinesterase], BCL10 [B-cell CLL/lymphoma 10], BCL2 [B-cell CLL/lymphoma 2], BCL2A1 [BCL2-related protein AI], BCL2L1 [BCL2-like 1], BCL2L11 [BCL2-like 11 (apoptosis facilitator)], BCL3 [B-cell CLL/lymphoma 3], BCL6 [B-cell CLL/lymphoma 6], BCL7A [B-cell CLL!lymphoma 7A], BCL7B [B-cell CLL!lymphoma 7B], BCL7C [B-cell CLL!lymphoma 70], BCR [breakpoint cluster region], BDKRB1 [bradykinin receptor BI], BDNF [brain-derived neurotrophic factor], BECN1 [beclin 1, autophagy related], BEST1 [bestrophin 1], BEX1 [brain expressed, X-linked 1], BEX2 [brain expressedX-linked 2], BGLAP [bone gamma-carboxyglutamate (gla) protein], BGN [biglycan], BID [BR3 interacting domain death agonist], BINI [bridging integrator 1], BIRC2 [baculoviral 1AP repeat-containing 2], BIRC3 [baculoviral 1AP repeat-containing 3], BIRC5 [baculoviral 1AP repeat-containing 5], BIRC7 [baculoviral 1AP repeat-containing 7], BLK [B lymphoid tyrosine kinase], BLVRB [biliverdin reductase B (flavin reductase (NADPR))], BMi1 [BMi1 polycomb ring finger oncogene], BMP1 [bone morphogenetic protein 1], BMP10 [bone morphogenetic protein 10], BMP15 [bone morphogenetic protein 15], BMP2 [bone morphogenetic protein 2], BMP3 [bone morphogenetic protein 3], BMP4 [bone morphogenetic protein 4], BMP5 [bone morphogenetic protein 5], BMP6 [bone morphogenetic protein 6], BMP7 [bone morphogenetic protein 7], BMPSA [bone morphogenetic protein Sa], BMPSB [bone morphogenetic protein 8b], BMPR1A [bone morphogenetic protein receptor, type IA], BMPR1B [bone morphogenetic protein receptor, type IB], BMPR2 [bone morphogenetic protein receptor, type II (serine/threonine kinase)], BOC [Boc homolog (mouse)], BOK [BCL2-related ovarian killer], BPI [bactericidal/permeability-increasing protein], BRAF [v-rafmurine sarcoma viral oncogene homolog BI], BRCA1 [breast cancer 1, early onset], BRCA2 [breast cancer 2, early onset], BRWD1 [bromodomain and WD repeat domain containing 1], BSND [Bartter syndrome, infantile, with sensorineural deafness (Barttin)], BST2 [bone marrow stromal cell antigen 2], BTBD1O [BTB (POZ) domain containing 10], BTC [betacellulin], BTD [biotinidase], BTG3 [BTG family, member 3], BTK [Bmton agannnaglobulinemia tyrosine kinase], BTN1A1 [butyrophilin, subfamily 1, member AI], BUB1B [budding uninhibited by benzimidazoles 1 homolog beta (yeast)], C15orf2 [chromosome 15 open reading frame 2], C16 or 175 [chromosome 16 open reading frame 75], C17orf42 [chromosome 17 open reading frame 42], C1orf187 [chromosome 1 open reading frame 187], C1R [complement component 1, r subcomponent], CIS [complement component 1, s subcomponent], C21orf2 [chromosome 21 open reading frame 2], C21orf33 [chromosome 21 open reading frame 33], C21orf45 [chromosome 21 open reading frame 45], C21orf62 [chromosome 21 open reading frame 62], C21orf74 [chromosome 21 open reading frame 74], C3 [complement component 3], C3orf58 [chromosome 3 open reading frame 58], C4A [complement component 4A (Rodgers blood group)], C4B [complement component 4B (Chido blood group)], C5AR1 [complement component Sa receptor 1], C6orf106 [chromosome 6 open reading frame 106], C6orf25 [chromosome 6 open reading frame 25], CA1 [carbonic anhydrase 1], CA2 [carbonic anhydrase II], CA3 [carbonic anhydrase III, muscle specific], CA6 [carbonic anhydrase VI], CA9 [carbonic anhydrase IX], CABIN1 [calcineurin binding protein I], CABLES1 [Cdk5 and Ab1 enzyme substrate 1], CACNA1B [calcium channel, voltage-dependent, N type, alpha 1B subunit], CACNA1C [calcium channel, voltage-dependent, L type, alpha IC subunit], CACNA1G [calcium channel, voltage-dependent, T type, alpha 1G subunit], CACNA1H [calcium channel, voltage-dependent, T type, alpha 1H subunit], CACNA2D1 [calcium channel, voltage-dependent, alpha 2/delta subunit 1], CADM1 [cell adhesion molecule 1], CADPS2 [Ca-++-dependent secretion activator 2], CALB2 [calbindin 2], CALCA [calcitonin-related polypeptide alpha], CALCR [calcitonin receptor], CALM3 [calmodulin 3 (phosphorylase kinase, delta)], CALR [calreticulin], CAMK1 [calcium/calmodulin-dependent protein kinase 1], CAMK2A [calcium/calmodulin-dependent protein kinase II alpha], CAMK2B [calcium/calmodulin-dependent protein kinase II beta], CAMK2G [calcium/calmodulin-dependent protein kinase II gamma], CAMK4 [calcium/calmodulin-dependent protein kinase N], CAMKK2 [calcium/calmodulin-dependent protein kinase kinase 2, beta], CAMP [cathelicidin antimicrobial peptide], CANT1 [calcium activated nucleotidase 1], CANX [calnexin], CAPN1 [calpain 1, (mull) large subunit], CAPN2 [calpain 2, (m/II) large subunit], CAPN5 [calpain 5], CAPZA1 [capping protein (actin filament) muscle Z-line, alpha 1], CARD16 [caspase recmitment domain family, member 16], CARMI [coactivator-associated arginine methyltransferase 1], CARTPT [CART prepropeptide], CASK [calcium/calmodulin-dependent serine protein kinase (MAGUK family)], CASP1 [caspase 1, apoptosis-related cysteine peptidase (interleukin 1, beta, convertase)], CASP10 [caspase 10, apoptosis-related cysteine peptidase], CASP2 [caspase 2, apoptosis-related cysteine peptidase], CASP3 [caspase 3, apoptosis-related cysteine peptidase], CASP6 [caspase 6, apoptosis-related cysteine peptidase], CASP7 [caspae 7, apoptosis-related cysteine peptidase], CASPS [caspase 8, apoptosis-related cysteine peptidase], CASP8AP2 [caspase 8 associated protein 2], CASP9 [caspase 9, apoptosis-related cysteine peptidase], CASR [calcium-sensing receptor], CAST [calpastatin], CAT [catalase], CAV1 [caveolin 1, caveolae protein, 22 kDa], CAV2 [caveolin 2], CAV3 [caveolin 3], CBL [Cas-Br-M (murine) ecotropic retroviral transforming sequence], CBLB [Cas-Br-M (murine) ecotropic retroviral transforming sequence b], CBR1 [carbonyl reductase I], CBR3 [carbonyl reductase 3], CBS [cystathionine-beta-synthase], CBXI [chromobox homolog I (HPI beta homolog Drosophila)], CBX5 [chromobox homolog 5 (HPI alpha homolog, Drosophila)], CC2D2A [coiled-coil and C2 domain containing 2A], CCBEI [collagen and calcium binding EGF domains I], CCBLI [cysteine conjugate-beta lyase, cytoplasmic], CCDC50 [coiled-coil domain containing 50], CCK [cholecystokinin], CCKAR [cholecystokinin A receptor], CCLI [chemokine (C-C motif) ligand I], CCLII [chemokine (C-C motif) ligand II], CCLI3 [chemokine (C-C motif) ligand 13], CCLI7 [chemokine (C-C motif) ligand 17], CCL19 [chemokine (C-C motif) ligand 19], CCL2 [chemokine (C-C motif) ligand 2], CCL20 [chemokine (C-C motif) ligand 20], CCL21 [chemokine (C-C motif) ligand 21], CCL22 [chemokine (C-C motif) ligand 22], CCL26 [chemokine (C-C motif) ligand 26], CCL27 [chemokine (C-C motif) ligand 27], CCL3 [chemokine (C-C motif) ligand 3], CCL4 [chemokine (C-C motif) ligand 4], CCL5 [chemokine (C-C motif) ligand 5], CCL7 [chemokine (C-C motif) ligand 7], CCLS [chemokine (C-C motif) ligand 8], CCNAI [cyclin AI], CCNA2 [cyclin A2], CCNBI [cyclin BI], CCNDI [cyclin DI], CCND2 [cyclin D2], CCND3 [cyclin D3], CCNG1 [cyclin GI], CCNH [cyclin H], CCNT1 [cyclin T1], CCR1 [chemokine (C-C motif) receptor 1], CCR3 [chemokine (C-C motif) receptor 3], CCR4 [chemokine (C-C motif) receptor 4], CCR5 [chemokine (C-C motif) receptor 5], CCR6 [chemokine (C-C motif) receptor 6], CCR7 [chemokine (C-C motif) receptor 7], CCT5 [chaperonin containing TCPI, subunit 5 (epsilon)], CDI4 [CDI4 molecule], CDI9 [CDI9 molecule], CDIA [CD I a molecule], CDIB [CDib molecule], CDID [CDid molecule], CD2 [CD2 molecule], CD209 [CD209 molecule], CD22 [CD22 molecule], CD244 [CD244 molecule, natural killer cell receptor 2B4], CD247 [CD247 molecule], CD27 [CD27 molecule], CD274 [CD274 molecule], CD28 [CD28 molecule], CD2AP [CD2-associated protein], CD33 [CD33 molecule], CD34 [CD34 molecule], CD36 [CD36 molecule (thrombospondin receptor)], CD3E [CD3e molecule, epsilon (CD3-TCR complex)], CD3G [CD3g molecule, gamma (CD3-TCRcomplex)], CD4 [CD4 molecule], CD40 [CD40 molecule, TNF receptor superfamily member 5], CD40LG [CD40 ligand], CD44 [CD44 molecule (Indian blood group)], CD46 [CD46 molecule, complement regulatory protein], CD47 [CD47 molecule], CD5 [CD5 molecule], CD55 [CD55 molecule, decay accelerating factor for complement (Cromer blood group)], CD58 [CD58 molecule], CD59 [CD59 molecule, complement regulatory protein], CD63 [CD63 molecule], CD69 [CD69 molecule], CD7 [CD7 molecule], CD72 [CD72 molecule], CD74 [CD74 molecule, major histocompatibility complex, class II invariant chain], CD79A [CD79a molecule, immunoglobulin-associated alpha], CD79B [CD79b molecule, immunoglobulin-associated beta], CD80 [CD80 molecule], CD8I [CD8I molecule], CD86 [CD86 molecule], CD8A [CD8a molecule], CD9 [CD9 molecule], CD99 [CD99 molecule], CDA [cytidine deaminase], CDC25A [cell division cycle 25 homolog A (S. pombe)], CDC25C [cell division cycle 25 homolog C (S. pombe)], CDC37 [cell division cycle 37 homolog (S. cerevisiae)], CDC42 [cell division cycle 42 (GTP binding protein, 25 kDa)], CDC5L [CDC5 cell division cycle 5-like (S. pombe)], CDH1 [cadherin 1, type I, E-cadherin (epithelial)], CDHIO [cadherin IO, type 2 (T2-cadherin)], CDHI2 [cadherin 12, type 2 (N-cadherin 2)], CDH15 [cadherin 15, type 1, M-cadherin (myotubule)], CDH2 [cadherin 2, type 1, N-cadherin (neuronal)], CDH4 [cadherin 4, type 1, R-cadherin (retinal)], CDH5 [cadherin 5, type 2 (vascular endothelium)], CDH9 [cadherin 9, type 2 (TI-cadherin)], CDIPT [CDP-diacylglycerol-inositol3-phosphatidyltransferase (phosphatidylinositol synthase)], CDK1 [cyclin-dependent kinase 1], CDK14 [cyclin-dependent kinase 14], CDK2 [cyclin-dependent kinase 2], CDK4 [cyclin-dependent kinase 4], CDK5 [cyclin-dependent kinase 5], CDK5R1 [cyclin-dependent kinase 5, regulatory subunit 1 (p35)], CDK5RAP2 [CDK5 regulatory subunit associated protein 2], CDK6 [cyclin-dependent kinase 6], CDK7 [cyclin-dependent kinase 7], CDK9 [cyclin-dependent kinase 9], CDKL5 [cyclin-dependent kinase-like 5], CDKN1A [cyclin-dependent kinase inhibitor 1A (p21, Cip1)], CDKN1B [cyclin-dependent kinase inhibitor 1B (p27, Kip1)], CDKN1C [cyclin-dependent kinase inhibitor IC (p57, Kip2)], CDKN2A [cyclin-dependent kinase inhibitor 2A (melanoma, p16, inhibits CDK4)], CDKN2B [cyclin-dependent kinase inhibitor 2B (p15, inhibits CDK4)], CDKN2C [cyclin-dependentkinaseinhibitor2C (p18, inhibits CDK4)], CDKN2D [cyclin-dependent kinase inhibitor 2D (p19, inhibits CDK4)], CDNF [cerebral dopamine neurotrophic factor], CDO1 [cysteine dioxygenase, type I], CDR2 [cerebellar degeneration-related protein 2, 62 kDa], CDT1 [chromatin licensing and DNA replication factor 1], CDX1 [caudal type homeobox 1], CDX2 [caudal type homeobox 2], CEACAM1 [carcinoembryonic antigen-related cell adhesion molecule 1 (bilimy glycoprotein)], CEACAM3 [carcinoembryonic antigen-related cell adhesion molecule 3], CEACAM5 [carcinoembryonic antigen-related cell adhesion molecule 5], CEACAM7 [carcinoembryonic antigen-related cell adhesion molecule 7], CEBPB [CCAAT/enhancer binding protein (C/EBP), beta], CEBPD [CCAAT/enhancer binding protein (C/EBP), delta], CECR2 [cat eye syndrome chromosome region, candidate 2], CEL [carboxyl ester lipase (bile salt-stimulated lipase)], CENPC1 [centromere protein C1], CENPJ [centromere protein J], CEP290 [centrosomal protein 290 kDa], CER1 [cerberus 1, cysteine knot superfamily, homolog (Xenopus laevis)], CETP [cholesteryl ester transfer protein, plasma], CFC1 [cripto, FRL-1, cryptic family 1], CFH [complement factor H], CFHRI [complement factor H-related 1], CFHR3 [complement factor H-related 3], CFHR4 [complement factor H-related 4], CFI [complement factor I], CFL1 [cofilin 1 (non-muscle)], CFL2 [cofilin 2 (muscle)], CFLAR [CASP8 and FADD-like apoptosis regulator], CFTR [cystic fibrosis transmembrane conductance regulator (ATP-binding cassette sub-family C, member 7)], CGA [glycoprotein hormones, alpha polypeptide], CGB [chorionic gonadotropin, beta polypeptide], CGB5 [chorionic gonadotropin, beta polypeptide 5], CGGBP1 [CGG triplet repeat binding protein 1], CHAF1A [chromatin assembly factor 1, subunit A (p150)], CHAF1B [chromatin assembly factor 1, subunit B (p60)], CHAT [choline acetyltransferase], CHEK1 [CHK1 checkpoint homolog (S. pombe)], CHEK2 [CHK2 checkpoint homolog (S. pombe)], CHGA [chromogranin A (parathyroid secretory protein 1)], CHKA [choline kinase alpha], CHL1 [cell adhesion molecule with homology to LICAM (close homolog of L1)], CHN1 [chimerin (chimaerin) 1], CHP [calcium binding protein P22], CHP2 [calcineurin B homologous protein 2], CHRD [chordin], CHRM1 [cholinergic receptor, muscarinic 1], CHRM2 [cholinergic receptor, muscarinic 2], CHRM3 [cholinergic receptor, muscarinic 3], CHRM5 [cholinergic receptor, muscarinic 5], CHRNA3 [cholinergic receptor, nicotinic, alpha 3], CHRNA4 [cholinergic receptor, nicotinic, alpha 4], CHRNA7 [cholinergic receptor, nicotinic, alpha 7], CHRNB2 [cholinergic receptor, nicotinic, beta 2 (neuronal)], CHST1 [carbohydrate (keratan sulfate Gal-6) sulfotransferase 1], CHST10 [carbohydrate sulfotransferase 10], CHST3 [carbohydrate (chondroitin 6) sulfotransferase 3], CHUK [conserved helix-loop-helix ubiquitous kinase], CHURCl [churchill domain containing 1], CIB1 [calcium and integrin binding 1 (calmyrin)], CIITA [class II, major histocompatibility complex, transactivator], CIRBP [cold inducible RNA binding protein], CISD1 [CDGSH iron sulfur domain 1], CISH [cytokine inducible SH2-containing protein], CIT [citron (rho-interacting, serine/threonine kinase 21)], CLASP2 [cytoplasmic linker associated protein 2], CLCF1 [cardiotrophin-like cytokine factor 1], CLCN2 [chloride channel2], CLDN1 [claudin 1], CLDN14 [claudin 14], CLDN16 [claudin 16], CLDN3 [claudin 3], CLDN4 [claudin 4], CLDN5 [claudin 5], CLDN8 [claudin 8], CLEC12A [C-type lectin domain family 12, member A], CLEC16A [C-type lectin domain family 16, member A], CLEC5A [C-type lectin domain family 5, member A], CLEC7A [C-type lectin domain family 7, member A], CLIP2 [CAP-GLY domain containing linker protein 2], CLSTN1 [calsyntenin 1], CLTC [clathrin, heavy chain (He)], CLU [clusterin], CMIP [c-Maf-inducing protein], CNBP [CCHC-type zinc finger, nucleic acid binding protein], CNGA3 [cyclic nucleotide gated channel alpha 3], CNGB3 [cyclic nucleotide gated channel beta 3], CNN1 [calponin 1, basic, smooth muscle], CNN2 [calponin 2], CNN3 [calponin 3, acidic], CNOT8 [CCR4-NOT transcription complex, subunit 8], CNP [2′ [3′-cyclic nucleotide 3′ phosphodiesterase], CNR1 [cannabinoid receptor 1 (brain)], CNR2 [cannabinoid receptor 2 (macrophage)], CNTF [ciliary neurotrophic factor], CNTFR [ciliary neurotrophic factor receptor], CNTFR [ciliary neurotrophic factor receptor], CNTFR [ciliary neurotrophic factor receptor], CNTLN [centlein, centrosomal protein], CNTN1 [contactin 1], CNTN2 [contactin 2 (axonal)], CNTN4 [contactin 4], CNTNAP1 [contactin associated protein 1], CNTNAP2 [contactin associated protein-like 2], COBL [cordon-bleu homolog (mouse)], COG2 [component of oligomeric golgi complex 2], COL18A1 [collagen, type XVIII, alpha 1], COL1A![collagen, type I, alpha 1], COLIA2 [collagen, type I, alpha 2], COL2A1 [collagen, type II, alpha 1], COL3A1 [collagen, type III, alpha 1], COL4A3 [collagen, type IV, alpha 3 (Goodpasture antigen)], COL4A3BP [collagen, type N, alpha 3 (Goodpasture antigen) binding protein], COL5A1 [collagen, type V, alpha 1], COL5A2 [collagen, type V, alpha 2], COL6A1 [collagen, type VI, alpha 1], COL6A2 [collagen, type VI, alpha 2], COL6A3 [collagen, type VI, alpha 3], COMT [catechol-0-methyltransferase], COPG2 [coatomer protein complex, subunit gamma 2], COPS4 [COPS constitutive photomorphogenic homolog subunit 4 (Arabidopsis)], COR01A [coronin, actin binding protein, 1A], COX5A [cytochrome c oxidase subunit Va], COX7B [cytochrome c oxidase subunit VIIb], CP [cemloplasmin (ferroxidase)], CPA1 [carboxypeptidase A1 (pancreatic)], CPA2 [carboxypeptidase A2 (pancreatic)], CPA5 [carboxypeptidase A5], CPB2 [carboxypeptidase B2 (plasma)], CPOX [coproporphyrinogen oxidase], CPS1 [carbamoyl-phosphate synthetase 1, mitochondrial], CPT1A [camitine palmitoyltransferase 1A (liver)], CR1 [complement component (3b/4b) receptor 1 (Knops blood group)], CR2 [complement component (3d/Epstein Barr vims) receptor 2], CRABP1 [cellular retinoic acid binding protein 1], CRABP2 [cellular retinoic acid binding protein 2], CRAT [camitine 0-acetyltransferase], CRB1 [crumbs homolog 1 (Drosophila)], CREB1 [cAMP responsive element binding protein 1], CREBBP [CREB binding protein], CRELD1 [cysteine-rich with EGF-like domains 1], CRH [corticotropin releasing hormone], CRIP1 [cysteine-rich protein 1 (intestinal)], CRK [v-crk sarcoma virus CTIO oncogene homolog (avian)], CRKL [v-crk sarcoma virus CTIO oncogene homolog (avian)-like], CRLF1 [cytokine receptor-like factor 1], CRLF2 [cytokine receptor-like factor 2], CRLF3 [cytokine receptor-like factor 3], CRMP1 [collapsin response mediator protein 1], CRP [C-reactive protein, pentraxin-related], CRTC1 [CREB regulated transcription coactivator 1], CRX [cone-rod homeobox], CRYAA [crystallin, alpha A], CRYAB [crystallin, alphaB], CS [citrate synthase], CSAD [cysteine sulfinic acid decarboxylase], CSF1 [colony stimulating factor 1 (macrophage)], CSF1R [colony stimulating factor 1 receptor], CSF2 [colony stimulating factor 2 (granulocyte-macrophage)], CSF2RA [colony stimulating factor 2 receptor, alpha, low-affinity (granulocyte-macrophage)], CSF3 [colony stimulating factor 3 (granulocyte)], CSF3R [colony stimulating factor 3 receptor (granulocyte)], CSH2 [chorionic somatomammotropin hormone 2], CSK [c-src tyrosine kinase], CSMD1 [CUB and Sushi multiple domains 1], CSMD3 [CUB and Sushi multiple domains 3], CSNK1D [casein kinase 1, delta], CSNKIE [casein kinase 1, epsilon], CSNK2A1 [casein kinase 2, alpha 1 polypeptide], CSPG4 [chondroitin sulfate proteoglycan 4], CSPG5 [chondroitin sulfate proteoglycan 5 (neuroglycan C)], CST3 [cystatin C], CST7 [cystatin F (leukocystatin)], CSTB [cystatin B (stefin B)], CTAG1B [cancer/testis antigen IB], CTBP1 [C-terminal binding protein 1], CTCF [CCCTC-binding factor (zinc finger protein)], CTDSP1 [CTD (carboxy-terminal domain, RNA polymerase II, polypeptide A) small phosphatase 1], CTF1 [cardiotrophin 1], CTGF [connective tissue growth factor], CTLA4 [cytotoxic T-lymphocyte-associated protein 4], CTNNA1 [catenin (cadherin-associated protein), alpha 1, 102 kDa], CTNNAL1 [catenin (cadherin-associated protein), alpha-like 1], CTNNB1 [catenin (cadherin-associated protein), beta 1, 88 kDa], CTNND1 [catenin (cadherin-associated protein), delta 1], CTNND2 [catenin (cadherin-associated protein), delta 2 (neural plakophilin-related arm-repeat protein)], CTNS [cystinosis, nephropathic], CTRL [chymotrypsin-like], CTSB [cathepsin B], CTSC [cathepsin C], CTSD [cathepsin D], CTSG [cathepsin G], CTSH [cathepsin H], CTSLI [cathepsin L], CTSS [cathepsin S], CTTN [cortactin], CTTNBP2 [cortactin binding protein 2], CUL4B [cullin 4B], CUL5 [cullin 5], CUX2 [cut-like homeobox 2], CX3CL1 [chemokine (C-X₃-C motif) ligand 1], CX3CR1 [chemokine (C-X₃-C motif) receptor 1], CXADR [coxsackie virus and adenovirus receptor], CXCLI [chemokine (C-X-C motif) ligand 1 (melanoma growth stimulating activity, alpha)], CXCLIO [chemokine (C-X-C motif) ligand 10], CXCL12 [chemokine (C-X-C motif) ligand 12 (stromal cell-derived factor 1)], CXCL16 [chemokine (C-X-C motif) ligand 16], CXCL2 [chemokine (C-X-C motif) ligand 2], CXCL5 [chemokine (C-X-C motif) ligand 5], CXCR1 [chemokine (C-X-C motif) receptor 1], CXCR2 [chemokine (C-X-C motif) receptor 2], CXCR3 [chemokine (C-X-C motif) receptor 3], CXCR4 [chemokine (C-X-C motif) receptor 4], CXCR5 [chemokine (C-X-C motif) receptor 5], CYB5A [cytochrome b5 type A (microsomal)], CYBA [cytochrome b-245, alpha polypeptide], CYBB [cytochrome b-245, beta polypeptide], CYCS [cytochrome c, somatic], CYFIP1 [cytoplasmic FMR1 interacting protein 1], CYLD [cylindromatosis (turban tumor syndrome)], CYP11A1 [cytochrome P450, family 11, subfamily A, polypeptide 1], CYP11B1 [cytochrome P450, family 11, subfamily B, polypeptide 1], CYP11B2 [cytochrome P450, family 11, subfamily B, polypeptide 2], CYP17A1 [cytochrome P450, family 17, subfamily A, polypeptide 1], CYP19A1 [cytochrome P450, family 19, subfamily A, polypeptide 1], CYP1A1 [cytochrome P450, family 1, subfamily A, polypeptide 1], CYP1A2 [cytochrome P450, family 1, subfamily A, polypeptide 2], CYP1B1 [cytochrome P450, family 1, subfamily B, polypeptide 1], CYP21A2 [cytochrome P450, family 21, subfamily A, polypeptide 2], CYP2A6 [cytochrome P450, family 2, subfamily A, polypeptide 6], CYP2B6 [cytochrome P450, family 2, subfamily B, polypeptide 6], CYP2C9 [cytochrome P450, family 2, subfamily C, polypeptide 9], CYP2D6 [cytochrome P450, family 2, subfamily D, polypeptide 6], CYP2E1 [cytochrome P450, family 2, subfamily E, polypeptide 1], CYP3A4 [cytochrome P450, family 3, subfamily A, polypeptide 4], CYP7A1 [cytochrome P450, family 7, subfamily A, polypeptide 1], CYR61 [cysteine-rich, angiogenic inducer, 61], CYSLTR1 [cysteinyl leukotriene receptor 1], CYSLTR2 [cysteinylleukotriene receptor 2], DAB1 [disabled homolog 1 (Drosophila)], DAGLA [diacylglycerol lipase, alpha], DAGLB [diacylglycerol lipase, beta], DAO [D-amino-acid oxidase], DAOA [D-amino acid oxidase activator], DAPK1 [death-associated protein kinase 1], DAPK3 [death-associated protein kinase 3], DAXX [death-domain associated protein], DBH [dopamine beta-hydroxylase (dopamine beta-monooxygenase)], DBI [diazepam binding inhibitor (GABA receptor modulator, acyl-Coenzyme A binding protein)], DBN1 [drebrin 1], DCAF6 [DDB1 and CUL4 associated factor 6], DCC [deleted in colorectal carcinoma], DCDC2 [doublecortin domain containing 2], DCK [deoxycytidine kinase], DCLK1 [doublecortin-like kinase 1], DCN [decorin], DCTN1 [dynactin 1 (p150, glued homolog, Drosophila)], DCTN2 [dynactin 2 (p50)], DCTN4 [dynactin 4 (p62)], DCUN1D1 [DCN1, defective in cullin neddylation 1, domain containing 1 (S. cerevisiae)], DCX [doublecortin], DDB1 [damage-specific DNA binding protein 1, 127 kDa], DDC [dopa decarboxylase (aromatic L-amina acid decarboxylase)], DDIT3 [DNA-damage-inducible transcript 3], DDIT4 [DNA-damage-inducible transcript 4], DDIT4L [DNA-damage-inducible transcript 4-like], DDRI [discoidin domain receptor tyrosine kinase 1], DDXIO [DEAD (Asp-Glu-Ala-Asp) box polypeptide 10], DDX17 [DEAD (Asp-Glu-Ala-Asp) box polypeptide 17], DEFB4A [defensin, beta 4A], DEK [DEK oncogene], DES [desmin], DEXI [Dexi homolog (mouse)], DFFA [DNA fragmentation factor, 45 kDa, alpha polypeptide], DFNB31 [deafness, autosomal recessive 31], DGCR6 [DiGeorge syndrome critical region gene 6], DGUOK [deoxyguanosine kinase], DHCR7 [7-dehydrocholesterol reductase], DHFR [dihydrofolate reductase], DIAPH1 [diaphanous homolog 1 (Drosophila)], DICER1 [dicer 1, ribonuclease type III], D101 [deiodinase, iodothyronine, type I], D102 [deiodinase, iodothyronine, type II], DIP2A [DIP2 disco-interacting protein 2 homolog A (Drosophila)], DIRAS3 [DIRAS family, GTP-binding RAS-like 3], DISC1 [dismpted in schizophrenia 1], DISC2 [dismpted in schizophrenia 2 (non-protein coding)], DKC1 [dyskeratosis congenita 1, dyskerin], DLG1 [discs, large homolog 1 (Drosophila)], DLG2 [discs, large homolog 2 (Drosophila)], DLG3 [discs, large homolog 3 (Drosophila)], DLG4 [discs, large homolog 4 (Drosophila)], DLGAP1 [discs, large (Drosophila) homolog-associated protein 1], DLGAP2 [discs, large (Drosophila) homolog-associated protein 2], DLK1 [delta-like 1 homolog (Drosophila)], DLL1 [delta-like 1 (Drosophila)], DLX1 [distal-less homeobox 1], DLX2 [distal-less homeobox 2], DLX3 [distal-less homeobox 3], DLX4 [distal-less homeobox 4], DLX5 [distal-less homeobox 5], DLX6 [distal-less homeobox 6], DMBT1 [deleted in malignant brain tumors 1], DMC1 [DMC1 dosage suppressor of mek1 homolog, meiosis-specific homologous recombination (yeast)], DMD [dystrophin], DMPK [dystrophia myotonica-protein kinase], DNAI2 [dynein, axonemal, intermediate chain 2], DNAJC28 [DnaJ (Hsp40) homolog, subfamily C, member 28], DNAJC30 [DnaJ (Hsp40) homolog, subfamily C, member 30], DNASE1 [deoxyribonuclease I], DNER [delta/notch-like EGF repeat containing], DNLZ [DNL-type zinc finger], DNM1 [dynamin 1], DNM3 [dynamin 3], DNMT1 [DNA (cytosine-5-)-methyltransferase 1], DNMT3A [DNA (cytosine-5-)-methyltransferase 3 alpha], DNMT3B [DNA (cytosine-5-)-methyltransferase 3 beta], DNTT [deoxynucleotidyltransferase, terminal], DOC2A [double C2-like domains, alpha], DOCK1 [dedicator of cytokinesis 1], DOCK3 [dedicator of cytokinesis 3], DOCK4 [dedicator of cytokinesis 4], DOCK7 [dedicator of cytokinesis 7], DOK7 [docking protein 7], DONSON [downstream neighbor of SON], DOPEY1 [dopey family member 1], DOPEY2 [dopey family member 2], DPF1 [D4, zinc and double PHD fingers family 1], DPF3 [D4, zinc and double PHD fingers, family 3], DPH1 [DPH1 homolog (S. cerevisiae)], DPP10 [dipeptidyl-peptidase 10], DPP4 [dipeptidyl-peptidase 4], DPRXP4 [divergent-paired related homeobox pseudogene 4], DPT [dermatopontin], DPYD [dihydropyrimidine dehydrogenase], DPYSL2 [dihydropyrimidinase-like 2], DPYSL3 [dihydropyrimidinase-like 3], DPYSL4 [dihydropyrimidinase-like 4], DPYSL5 [dihydropyrimidinase-like 5], DRD1 [dopamine receptor D1], DRD2 [dopamine receptor D2], DRD3 [dopamine receptor D3], DRD4 [dopamine receptor D4], DRD5 [dopamine receptor D5], DRG1 [developmentally regulated GTP binding protein 1], DRGX [dorsal root ganglia homeobox], DSC2 [desmocollin 2], DSCAM [Down syndrome cell adhesion molecule], DSCAML1 [Down syndrome cell adhesion molecule like 1], DSCR3 [Down syndrome critical region gene 3], DSCR4 [Down syndrome critical region gene 4], DSCR6 [Down syndrome critical region gene 6], DSERG1 [Down syndrome encephalopathy related protein 1], DSG1 [desmoglein 1], DSG2 [desmoglein 2], DSP [desmoplakin], DST [dystonin], DSTN [destrin (actin depolymerizing factor)], DTNBP1 [dystrobrevin binding protein 1], DULLARD [dullard homolog (Xenopus laevis)], DUSP1 [dual specificity phosphatase 1], DUSP13 [dual specificity phosphatase 13], DUSP6 [dual specificity phosphatase 6], DUT [deoxyuridine triphosphatase], DVL1 [dishevelled, dsh homolog 1 (Drosophila)], DYRKIA [dual-pecificity tyrosine-(Y)-phosphorylation regulated kinase 1A], DYRK3 [dual-specificity tyrosine-(Y)-phosphorylation regulated kinase 3], DYSF [dysferlin, limb girdle muscular dystrophy 2B (autosomal recessive)], DYX1C1 [dyslexia susceptibility 1 candidate 1], E2F1 [E2F transcription factor 1], EARS2 [glutamyl-tRNA synthetase 2, mitochondrial (putative)], EBF4 [early B-cell factor 4], ECE1 [endothelin converting enzyme 1], ECHS1 [enoyl Coenzyme A hydratase, short chain, 1, mitochondrial], EDN1 [endothelin 1], EDN2 [endothelin 2], EDN3 [endothelin 3], EDNRA [endothelin receptor type A], EDNRB [endothelin receptor type B], EEF1A1 [eukaryotic translation elongation factor 1 alpha 1], EEF2 [eukaryotic translation elongation factor 2], EEF2K [eukaryotic elongation factor-2 kinase], EFHA1 [EF-hand domain family, member A1], EFNA1 [ephrin-A1], EFNA2 [ephrin-A2], EFNA3 [ephrin-A3], EFNA4 [ephrin-A4], EFNA5 [ephrin-A5], EFNB2 [ephrin-B2], EFNB3 [ephrin-B3], EFS [embryonal Fyn-associated substrate], EGF [epidermal growth factor (beta-urogastrone)], EGFR [epidermal growth factorreceptor (erythroblastic leukemia viral (v-erb-b) oncogene homolog, avian)], EGLN1 [eg1 nine homolog 1 (C. elegans)], EGR1 [early growth response 1], EGR2 [early growth response 2], EGR3 [early growth response 3], EHHADH [enoyl-Coenzyme A, hydratase/3-hydroxyacyl Coenzyme A dehydrogenase], EHMT2 [euchromatic histone-lysine N-methyltransferase 2], EID1 [EP300 interacting inhibitor of differentiation 1], E1F 1AY [eukaryotic translation initiation factor 1A, Y-linked], EIF2AK2 [eukaryotic translation initiation factor 2-alpha kinase 2], EIF2AK3 [eukaryotic translation initiation factor 2-alpha kinase 3], EIF2B2 [eukaryotic translation initiation factor 2B, subunit 2 beta, 39 kDa], ETF2B5 [eukaryotic translation initiation factor 2B, subunit 5 epsilon, 82 kDa], ETF2S1 [eukaryotic translation initiation factor 2, subunit 1 alpha, 35 kDa], EIF2S2 [eukaryotic translation initiation factor 2, subunit 2 beta, 38 kDa], EIF3M [eukaryotic translation initiation factor 3, subunit M], EIF4E [eukaryotic translation initiation factor 4E], EIF4EBP1 [eukaryotic translation initiation factor 4E binding protein 1], EIF4G1 [eukaryotic translation initiation factor 4 gamma, 1], EIF4H [eukaryotic translation initiation factor 4H], ELANE [elastase, neutrophil expressed], ELAVL1 [ELAV (embryonic lethal, abnormal vision, Drosophila)-like 1 (Hu antigen R)], ELAVL3 [ELAV (embryonic lethal, abnormal vision, Drosophila)-like 3 (Hu antigen C)], ELAVL4 [ELAV (embryonic lethal, abnormal vision, Drosophila)-like 4 (Hu antigen D)], ELF5 [E74-like factor 5 (ets domain transcription factor)], ELK1 [ELK1, member of ETS oncogene family], ELMO I [engulfment and cell motility 1], ELN [elastin], ELP4 [elongation protein 4 homolog (S. cerevisiae)], EMP2 [epithelial membrane protein 2], EMP3 [epithelial membrane protein 3], EMX1 [empty spiracles homeobox 1], EMX2 [empty spiracles homeobox 2], EN1 [engrailed homeobox 1], EN2 [engrailed homeobox 2], ENAH [enabled homolog (Drosophila)], ENDOG [endonuclease G], ENG [endoglin], ENO1 [enolase 1, (alpha)], EN02 [enolase 2 (gamma, neuronal)], ENPEP [glutamyl aminopeptidase (aminopeptidase A)], ENPP1 [ectonucleotide pyrophosphatase/phosphodiesterase 1], ENPP2 [ectonucleotide pyrophosphatase/phosphodiesterase 2], ENSA [endosulfine alpha], ENSG00000174496 [ ], ENSG00000183653 [ ], ENSG00000215557 [ ], ENTPD1 [ectonucleoside triphosphate diphosphohydrolase 1], EP300 [E1A binding protein p300], EPCAM [epithelial cell adhesion molecule], EPHA1 [EPH receptor AI], EPHAIO [EPH receptor AIO], EPHA2 [EPH receptor A2], EPHA3 [EPH receptor A3], EPHA4 [EPH receptor A4], EPHA5 [EPH receptor AS], EPHA6 [EPH receptor A6], EPHA7 [EPH receptor A7], EPHA8 [EPH receptor A8], EPHB1 [EPH receptor B1], EPHB2 [EPH receptor B2], EPHB3 [EPH receptor B3], EPHB4 [EPH receptor B4], EPHB6 [EPH receptor B6], EPHX2 [epoxide hydrolase 2, cytoplasmic], EPM2A [epilepsy, progressive myoclonus type 2A, Lafora disease (laforin)], EPO [erythropoietin], EPOR [erythropoietin receptor], EPRS [glutamyl-prolyl-tRNA synthetase], EPS15 [epidermal growth factor receptor pathway substrate 15], ERBB2 [v-erb-b2 erythroblastic leukemia viral oncogene homolog 2, neuro/glioblastoma derived oncogene homolog (avian)], ERBB3 [v-erb-b2 erythroblastic leukemia viral oncogene homolog 3 (avian)], ERBB4 [v-erb-a erythroblastic leukemia viral oncogene homolog 4 (avian)], ERC2 [ELKS/RAB6-interacting/CAST family member 2], ERCC2 [excision repair cross-complementing rodent repair deficiency, complementation group 2], ERCC3 [excision repair cross-complementing rodent repair deficiency, complementation group 3 (xeroderma pigmentosum group B complementing)], ERCC5 [excision repair cross-complementing rodent repair deficiency, complementation group 5], ERCC6 [excision repair cross-complementing rodent repair deficiency, complementation group 6], ERCC8 [excision repair cross-complementing rodent repair deficiency, complementation group 8], EREG [epiregulin], ERG [v-ets erythroblastosis virus E26 oncogene homolog (avian)], ERVWE1 [endogenous retroviral family W, env(C7), member 1], ESD [esterase D/formylglutathione hydrolase], ESR1 [estrogen receptor 1], ESR2 [estrogen receptor 2 (ER beta)], ESRRA [estrogen-related receptor alpha], ESRRB [estrogen-related receptor beta], ETS1 [v-ets erythroblastosis virus E26 oncogene homolog 1 (avian)], ETS2 [v-ets erythroblastosis virus E26 oncogene homolog 2 (avian)], ETV1 [ets variant 1], ETV4 [ets variant 4], ETV5 [ets variant 5], ETV6 [ets variant 6], EVL [Enah/Vasp-like], EXOC4 [exocyst complex component 4], EXOC8 [exocyst complex component 8], EXT1 [exostoses (multiple) 1], EXT2 [exostoses (multiple) 2], EZH2 [enhancer ofzeste homolog 2 (Drosophila)], EZR [ezrin], F12 [coagulation factor XII (Hageman factor)], F2 [coagulation factor TT (thrombin)], F2R [coagulation factor TT (thrombin) receptor], F2RL1 [coagulation factor TT (thrombin) receptor-like 1], F3 [coagulation factor III (thromboplastin, tissue factor)], F7 [coagulation factor VII (serum prothrombin conversion accelerator)], F8 [coagulation factor VIII, procoagulant component], F9 [coagulation factor IX], FAAH [fatty acid amide hydrolase], FABP3 [fatty acid binding protein 3, muscle and heart (mammary-derived growth inhibitor)], FABP4 [fatty acid binding protein 4, adipocyte], FABP5 [fatty acid binding protein 5 (psoriasis-associated)], FABP7 [fatty acid binding protein 7, brain], FADD [Fas (TNFRSF6)-associated via death domain], FADS2 [fatty acid desaturase 2], FAM120C [family with sequence similarity 120C], FAM165B [family with sequence similarity 165, member B], FAM3C [family with sequence similarity 3, member C], FAM53A [family with sequence similarity 53, member A], FARP2 [FERM, RhoGEF and pleckstrin domain protein 2], FARSA [phenylalanyl-tRNA synthetase, alpha subunit], FAS [Fas (TNF receptor superfamily, member 6)], FASLG [Fas ligand (TNF superfamily, member 6)], FASN [fatty acid synthase], FASTK [Pas-activated serine/threonine kinase], FBLN1 [fibulin 1], FBN1 [fibrillin 1], FBP1 [fructose-I [6-bisphosphatase 1], FBX045 [F-box protein 45], FBXW5 [F-box and WD repeat domain containing 5], FBXW7 [F-box and WD repeat domain containing 7], FCER2 [Fe fragment oflgE, low affinity II, receptor for (CD23)], FCGR1A [Fe fragment oflgG, high affinity Ia, receptor (CD64)], FCGR2A [Fe fragment oflgG, low affinity I1a, receptor (CD32)], FCGR2B [Fe fragment oflgG, low affinity lib, receptor (CD32)], FCGR3A [Fe fragment oflgG, low affinity Ilia, receptor (CD16a)], FCRL3 [Fe receptor-like 3], FDFT1 [farnesyl-diphosphate farnesyltransferase 1], FDX1 [ferredoxin 1], FDXR [ferredoxin reductase], FECH [ferrochelatase (protoporphyria)], FEMIA [fem-1 homolog a (C. elegans)], FER [fer (fps/fes related) tyrosine kinase], FES [feline sarcoma oncogene], FEZ1 [fasciculation and elongation protein zeta 1 (zygin I)], FEZ2 [fasciculation and elongation protein zeta 2 (zygin II)], FEZF1 [FEZ family zinc finger 1], FEZF2 [FEZ family zinc finger 2], FGF1 [fibroblast growth factor 1 (acidic)], FGF19 [fibroblast growth factor 19], FGF2 [fibroblast growth factor 2 (basic)], FGF20 [fibroblast growth factor 20], FGF3 [fibroblast growth factor 3 (murine mammary tumor vims integration site (v-int-2) oncogene homolog)], FGF4 [fibroblast growth factor 4], FGF5 [fibroblast growth factor 5], FGF7 [fibroblast growth factor 7 (keratinocyte growth factor)], FGFS [fibroblast growth factorS (androgen-induced)], FGF9 [fibroblast growth factor 9 (glia-activating factor)], FGFBP1 [fibroblast growth factor binding protein 1], FGFR1 [fibroblast growth factor receptor 1], FGFR2 [fibroblast growth factor receptor 2], FGFR3 [fibroblast growth factor receptor 3], FGFR4 [fibroblast growth factor receptor 4], FHIT [fragile histidine triad gene], FHL1 [four and a half LIM domains 1], FHL2 [four and a half LIM domains 2], FIBP [fibroblast growth factor (acidic) intracellular binding protein], FIGF [c-fos induced growth factor (vascular endothelial growth factor D)], FTGNL1 [fidgetin-like 1], FKBP15 [FK506 binding protein 15, 133 kDa], FKBP1B [FK506 binding protein IB, 12.6 kDa], FKBP5 [FK506 binding protein 5], FKBP6 [FK506 binding protein 6, 36 kDa], FKBP8 [FK506 binding protein 8, 38 kDa], FKTN [fukutin], FLCN [folliculin], FLG [filaggrin], FLi1 [Friend leukemia vims integration 1], FLNA [filamin A, alpha], FLNB [filamin B, beta], FLNC [filamin C, gall1111a], FLT1 [fins-related tyrosine kinase 1 (vascular endothelial growth factor/vascular permeability factor receptor)], FLT3 [fins-related tyrosine kinase 3], FMN1 [fonnin 1], FMNL2 [fonnin-like 2], FMR1 [fragile X mental retardation 1], FN1 [fibronectin1], FOLH1 [folate hydrolase (prostate-specific membrane antigen) 1], FOLR1 [folate receptor 1 (adult)], FOS [FBJ murine osteosarcoma viral oncogene homolog], FOSB [FBJ murine osteosarcoma viral oncogene homolog B], FOXC2 [forkhead box C2 (MFH-1, mesenchyme forkhead 1)], FOXG1 [forkhead box GI], FOXL2 [forkhead box L2], FOXM1 [forkhead box M1], FOX01 [forkhead box O1], FOX03 [forkhead box O3], FOXP2 [forkhead box P2], FOXP3 [forkhead box P3], FPR1 [formyl peptide receptor 1], FPR2 [formyl peptide receptor 2], FRMD7 [FERM domain containing 7], FRS2 [fibroblast growth factor receptor substrate 2], FRS3 [fibroblast growth factor receptor substrate 3], FRYL [FRY-like], FSCN1 [fascin homolog 1, actin-bundling protein (Strongylocentrotus purpuratus)], FSHB [follicle stimulating hormone, beta polypeptide], FSHR [follicle stimulating hormone receptor], FST [follistatin], FSTL1 [follistatin-like 1], FSTL3 [follistatin-like 3 (secreted glycoprotein)], FTCD [formiminotransferase cyclodeaminase], FTH1 [ferritin, heavy polypeptide 1], FTL [ferritin, light polypeptide], FTMT [ferritin mitochondrial], FTSJI [FtsJ homolog 1 (E. coli)], FUCA1 [fucosidase, alpha-L-1, tissue], FURIN [furin (paired basic amino acid cleaving enzyme)], FUT1 [fucosyltransferase 1 (galactoside 2-alpha-L-fucosyltransferase, H blood group)], FUT4 [fucosyltransferase 4 (alpha (1 [3) fucosyltransferase, myeloid-specific)], FXN [frataxin], FXR1 [fragile X mental retardation, autosomal homolog 1], FXR2 [fragile X mental retardation, autosomal homolog 2], FXYD1 [FXYD domain containing ion transport regulator 1], FYB [FYN binding protein (FYB-120/130)], FYN [FYN oncogene related to SRC, FGR, YES], FZD1 [frizzled homolog 1 (Drosophila)], FZD10 [frizzled homolog 10 (Drowphila)], FZD2 [frizzled homolog 2 (Drosophila)], FZD3 [frizzled homolog 3 (Drosophila)], FZD4 [frizzled homolog 4 (Drosophila)], FZD5 [frizzled homolog 5 (Drosophila)], FZD6 [frizzled homolog 6 (Drosophila)], FZD7 [frizzled homolog 7 (Drosophila)], FZD8 [frizzled homolog 8 (Drosophila)], FZD9 [frizzled homolog 9 (Drosophila)], FZR1 [fizzy/cell division cycle 20 related 1 (Drosophila)], G6PD [glucose-6-phosphate dehydrogenase], GAA [glucosidase, alpha; acid], GAB1 [GRB2-associated binding protein1], GABARAP [GABA(A) receptor-associated protein], GABBR1 [gamma-aminobutyric acid (GABA) B receptor, 1], GABBR2 [gamma-aminobutyric acid (GABA) B receptor, 2], GABPA [GA binding protein transcription factor, alpha subunit 60 kDa], GABRA1 [gamma-aminobutyric acid (GABA) A receptor, alpha 1], GABRA2 [gamma-aminobutyric acid (GABA) A receptor, alpha 2], GABRA3 [gamma-aminobutyric acid (GABA) A receptor, alpha 3], GABRA4 [gamma-aminobutyric acid (GABA) A receptor, alpha 4], GABRA5 [gamma-aminobutyric acid (GABA) A receptor, alpha 5], GABRA6 [gamma-aminobutyric acid (GABA) A receptor, alpha 6], GABRB1 [gamma-aminobutyric acid (GABA) A receptor, beta 1], GABRB2 [gamma-aminobutyric acid (GABA) A receptor, beta 2], GABRB3 [gamma-aminobutyric acid (GABA) A receptor, beta 3], GABRD [gamma-aminobutyric acid (GABA) A receptor, delta], GABRE [gamma-aminobutyric acid (GABA) A receptor, epsilon], GABRG1 [gamma-aminobutyric acid (GABA) A receptor, gamma 1], GABRG2 [gamma-aminobutyric acid (GABA) A receptor, gamma 2], GABRG3 [gamma-aminobutyric acid (GABA) A receptor, gamma 3], GABRP [gamma-aminobutyric acid (GABA) A receptor, pi], GAD1 [glutamate decarboxylase 1 (brain, 67 kDa)], GAD2 [glutamate decarboxylase 2 (pancreatic islets and brain, 65 kDa)], GAL [galanin prepropeptide], GALE [UDP-galactose-4-epimerase], GALK1 [galactokinase 1], GALT [galactose-1-phosphate uridylyltransferase], GAP43 [growth associated protein 43], GAPDH [glyceraldehyde-3-phosphate dehydrogenase], GARS [glycyl-tRNA synthetase], GART [phosphoribosylglycinamide formyltransferase, phosphoribosylglycinamide synthetase, phosphoribosylaminoimidazole synthetase], GAS1 [growth arrest-specific 1], GAS6 [growth arrest-specific 6], GAST [gastrin], GATA1 [GATA binding protein 1 (globin transcription factor 1)], GATA2 [GATA binding protein 2], GATA3 [GATA binding protein 3], GATA4 [GATA binding protein4], GATA6 [GATA binding protein 6], GBA [glucosidase, beta, acid], GBE1 [glucan (1 [4-alpha-), branching enzyme 1], GBX2 [gastrulation brain homeobox 2], GC [group-specific component (vitamin D binding protein)], GCG [glucagon], GCH1 [GTP cyclohydrolase 1], GCNT1 [glucosaminyl (N-acetyl) transferase 1, core 2], GDAP1 [ganglioside-induced differentiation-associated protein 1], GDF1 [growth differentiation factor 1], GDF11 [growth differentiation factor 11], GDF15 [growth differentiation factor 15], GDF7 [growth differentiation factor 7], GDi1 [GDP dissociation inhibitor 1], GDI2 [GDP dissociation inhibitor 2], GDNF [glial cell derived neurotrophic factor], GDPD5 [glycerophosphodiester phosphodiesterase domain containing 5], GEM [GTP binding protein overexpressed in skeletal muscle], GFAP [glial fibrillary acidic protein], GFER [growth factor, augmenter of liver regeneration], GFi1B [growth factor independent 1B transcription repressor], GFRA1 [GDNF family receptor alpha 1], GFRA2 [GDNF family receptor alpha 2], GFRA3 [GDNF family receptor alpha 3], GFRA4 [GDNF family receptor alpha 4], GGCX [gamma-glutamyl carboxylase], GGNBP2 [gametogenetin binding protein2], GGT1 [gamma-glutamyltransferase 1], GGT2 [gamma-glutamyltransferase 2], GH1 [growth hormone 1], GHR [growth hormone receptor], GHRH [growth hormone releasing hormone], GHRHR [growth hormone releasing hormone receptor], GHRL [ghrelin!obestatin prepropeptide], GHSR [growth hormone secretagogue receptor], GIPR [gastric inhibitory polypeptide receptor], GIT1 [G protein-coupled receptor kinase interacting ArfGAP 1], GJA1 [gap junction protein, alpha 1, 43 kDa], GJA4 [gap junction protein, alpha 4, 37 kDa], GJA5 [gap junction protein, alpha 5, 40 kDa], GJB1 [gap junction protein, beta 1, 32 kDa], GJB2 [gap junction protein, beta 2, 26 kDa], GJB6 [gap junction protein, beta 6, kDa], GLA [galactosidase, alpha], GLB1 [galactosidase, beta 1], GLDC [glycine dehydrogenase (decarboxylating)], GLI1 [GLI family zinc finger 1], GLI2 [GLI family zinc finger 2], GLI3 [GLI family zinc finger 3], GLIS1 [GLIS family zinc finger 1], GLIS2 [GLIS family zinc finger 2], GL01 [glyoxalase I], GLRA2 [glycine receptor, alpha 2], GLRB [glycine receptor, beta], GLS [glutaminase], GLUDI [glutamate dehydrogenase 1], GLUD2 [glutamate dehydrogenase 2], GLUL [glutamate-ammonia ligase (glutamine synthetase)], GL YAT [glycine-N-acyltransferase], GMFB [glia maturation factor, beta], GMNN [geminin, DNA replication inhibitor], GMPS [guanine monophosphate synthetase], GNA11 [guanine nucleotide binding protein (G protein), alpha 11 (Gq class)], GNA12 [guanine nucleotide binding protein (G protein) alpha 12], GNA13 [guanine nucleotide binding protein (G protein), alpha 13], GNA14 [guanine nucleotide binding protein (G protein), alpha 14], GNA15 [guanine nucleotide binding protein (G protein), alpha 15 (Gq class)], GNAI1 [guanine nucleotide binding protein (G protein), alpha inhibiting activity polypeptide 1], GNAT2 [guanine nucleotide binding protein (G protein), alpha inhibiting activity polypeptide 2], GNAI3 [guanine nucleotide binding protein (G protein), alpha inhibiting activity polypeptide 3], GNAL [guanine nucleotide binding protein (G protein), alpha activating activity polypeptide, olfactory type], GNA01 [guanine nucleotide binding protein (G protein), alpha activating activity polypeptide 0], GNAQ [guanine nucleotide binding protein (G protein), q polypeptide], GNAS [GNAS complex locus], GNAT1 [guanine nucleotide binding protein (G protein), alpha transducing activity polypeptide 1], GNAT2 [guanine nucleotide binding protein (G protein), alpha transducing activity polypeptide 2], GNAZ [guanine nucleotide binding protein (G protein), alpha z polypeptide], GNB1 [guanine nucleotide binding protein (G protein), beta polypeptide 1], GNB1L [guanine nucleotide binding protein (G protein), beta polypeptide 1-like], GNB2 [guanine nucleotide binding protein (G protein), beta polypeptide 2], GNB2L1 [guanine nucleotide binding protein (G protein), beta polypeptide 2-like 1], GNB3 [guanine nucleotide binding protein (G protein), beta polypeptide 3], GNB4 [guanine nucleotide binding protein (G protein), beta polypeptide 4], GNB5 [guanine nucleotide binding protein (G protein), beta 5], GNG10 [guanine nucleotide binding protein (G protein), gamma 10], GNG11 [guanine nucleotide binding protein (G protein), gamma 11], GNG12 [guanine nucleotide binding protein (G protein), gamma 12], GNG13 [guanine nucleotide binding protein (G protein), gamma 13], GNG2 [guanine nucleotide binding protein (G protein), gamma 2], GNG3 [guanine nucleotide binding protein (G protein), gamma 3], GNG4 [guanine nucleotide binding protein (G protein), gamma 4], GNG5 [guanine nucleotide binding protein (G protein), gamma 5], GNG7 [guanine nucleotide binding protein (G protein), gamma 7], GNLY [granulysin], GNRH1 [gonadotropin-releasing hormone 1 (luteinizing-releasing hormone)], GNRHR [gonadotropin-releasing hormone receptor], GOLGA2 [golgin A2], GOLGA4 [golgin A4], GOT2 [glutamic-oxaloacetic transaminase 2, mitochondrial (aspartate aminotransferase 2)], GPIBA [glycoprotein Ib (platelet), alpha polypeptide], GP5 [glycoprotein V (platelet)], GP6 [glycoprotein VI (platelet)], GP9 [glycoprotein IX (platelet)], GPC1 [glypican 1], GPC3 [glypican 3], GPD1 [glycerol-3-phosphate dehydrogenase 1 (soluble)], GPHN [gephyrin], GPI [glucose phosphate isomerase], GPM6A [glycoprotein M6A], GPM6B [glycoprotein M6B], GPR161 [G protein-coupled receptor 161], GPR182 [G protein-coupled receptor 182], GPR56 [G protein-coupled receptor 56], GPRC6A [G protein-coupled receptor, family C, group 6, member A], GPRIN1 [G protein regulated inducer of neurite outgrowth 1], GPT [glutamic-pyruvate transaminase (alanine aminotransferase)], GPT2 [glutamic pyruvate transaminase (alanine aminotransferase) 2], GPX1 [glutathione peroxidase 1], GPX3 [glutathione peroxidase 3 (plasma)], GPX4 [glutathione peroxidase 4 (phospholipid hydroperoxidase)], GRAP [GRB2-related adaptor protein], GRB10 [growth factor receptor-bound protein 10], GRB2 [growth factor receptor-bound protein 2], GRB7 [growth factor receptor-bound protein 7], GREM1 [gremlin 1, cysteine knot superfamily, homolog (Xenopus laevis)], GRIA1 [glutamate receptor, ionotropic, AMPA 1], GRIA2 [glutamate receptor, ionotropic, AMPA 2], GRIA3 [glutamate receptor, ionotrophic, AMPA 3], GRID2 [glutamate receptor, ionotropic, delta 2], GRID21P [glutamate receptor, ionotropic, delta 2 (Grid2) interacting protein], GRIK1 [glutamate receptor, ionotropic, kainate 1], GRIK2 [glutamate receptor, ionotropic, kainate 2], GRTN1 [glutamate receptor, ionotropic, N-methyl D-aspartate 1], GRTN2A [glutamate receptor, ionotropic, N-methyl D-aspartate 2A], GRIP I [glutamate receptor interacting protein 1], GRLF1 [glucocorticoid receptor DNA binding factor 1], GRM1 [glutamate receptor, metabotropic 1], GRM2 [glutamate receptor, metabotropic 2], GRM5 [glutamate receptor, metabotropic 5], GRM7 [glutamate receptor, metabotropic 7], GRM8 [glutamate receptor, metabotropic 8], GRN [granulin], GRP [gastrin-releasing peptide], GRPR [gastrin-releasing peptide receptor], GSK3B [glycogen synthase kinase 3 beta], GSN [gelsolin], GSR [glutathione reductase], GSS [glutathione synthetase], GSTA1 [glutathione S-transferase alpha 1], GSTM1 [glutathione S-transferase mu 1], GSTP1 [glutathione S-transferase pi 1], GSTT1 [glutathione S-transferase theta 1], GSTZ1 [glutathione transferase zeta 1], GTF2B [general transcription factor liB], GTF2E2 [general transcription factor liE, polypeptide 2, beta 34 kDa], GTF2H1 [general transcription factor IIH, polypeptide 1, 62 kDa], GTF2H2 [general transcription factor IIH, polypeptide 2, 44 kDa], GTF2H3 [general transcription factor IIH, polypeptide 3, 34 kDa], GTF2H4 [general transcription factor IIH, polypeptide 4, 52 kDa], GTF2I [general transcription factor IIi], GTF2IRD1 [GTF2I repeat domain containing 1], GTF2IRD2 [GTF2I repeat domain containing 2], GUCA2A [guanylate cyclase activator 2A (guanylin)], GUCY1A3 [guanylate cyclase 1, soluble, alpha 3], GUSB [glucuronidase, beta], GYPA [glycophorin A (MNS blood group)], GYPC [glycophorin C (Gerbich blood group)], GZFl [GDNF-inducible zinc finger protein 1], GZMA [granzyme A (granzyme 1, cytotoxic T-lymphocyte-associated serine esterase 3)], GZMB [granzyme B (granzyme 2, cytotoxic T-lymphocyte-associated serine esterase 1)], H19 [H19, imprinted maternally expressed transcript (non-protein coding)], H1F0 [H1 histone family, member 0], H2AFX [H2A histone family, member X], H2AFY [H2A histone family, member Y], H6PD [hexose-6-phosphate dehydrogenase (glucose}-dehydrogenase)], HADHA [hydroxyacyl-Coenzyme A dehydrogenase/3-ketoacyl-Coenzyme A thiolase/enoyl-Coenzyme A hydratase (trifunctional protein), alpha subunit], HAMP [hepcidin antimicrobial peptide], HAND1 [heart and neural crest derivatives expressed 1], HAND2 [hemi and neural crest derivatives expressed 2], HAP1 [huntingtin-associated protein 1], HAPLN1 [hyaluronan and proteoglycan link protein 1], HARS [histidyl-tRNA synthetase], HAS1 [hyaluronan synthase 1], HAS2 [hyaluronan synthase 2], HAS3 [hyaluronan synthase 3], HAX1 [HCLS1 associated protein X-1], HBA2 [hemoglobin, alpha 2], HBB [hemoglobin, beta], HBEGF [heparin-binding EGF-like growth factor], HBG1 [hemoglobin, gamma A], HBG2 [hemoglobin, gamma G], HCCS [holocytochrome c synthase (cytochrome c heme-lyase)], HCK [hemopoietic cell kinase], HCLS1 [hematopoietic cell-specific Lyn substrate 1], HCN4 [hyperpolarization activated cyclic nucleotide-gated potassium channel4], HCRT [hypocretin (orexin) neuropeptide precursor], HCRTR1 [hypocretin (orexin) receptor 1], HCRTR2 [hypocretin (orexin) receptor 2], HDAC1 [histone deacetylase 1], HDAC2 [histone deacetylase 2], HDAC4 [histone deacetylase 4], HDAC9 [histone deacetylase 9], HDC [histidine decarboxylase], HDLBP [high density lipoprotein binding protein], HEPACAM [hepatocyte cell adhesion molecule], HES1 [hairy and enhancer of split 1, (Drosophila)], HES3 [hairy and enhancer of split 3 (Drosophila)], HES5 [hairy and enhancer of split 5 (Drosophila)], HES6 [hairy and enhancer of split 6 (Drosophila)], HEXA [hexosaminidase A (alpha polypeptide)], HFE [hemochromatosis], HFE2 [hemochromatosis type 2 Guvenile)], HGF [hepatocyte growth factor (hepapoietin A; scatter factor)], HGS [hepatocyte growth factor-regulated tyrosine kinase substrate], HHEX [hematopoietically expressed homeobox], HHIP [hedgehog interacting protein], HIF1A [hypoxia inducible factor 1, alpha subunit (basic helix-loop-helix transcription factor)], HINT1 [histidine triad nucleotide binding protein 1], HIPK2 [homeodomain interacting protein kinase 2], HIRA [HIR histone cell cycle regulation defective homolog A (S. cerevisiae)], HIRIP3 [HIRA interacting protein 3], H1ST1H2AB [histone cluster 1, H2ab], H1ST1H2AC [histone cluster 1, H2ac], H1ST1H2AD [histone cluster 1, H2ad], H1ST1H2AE [histone cluster 1, H2ae], H1ST1H2AG [histone cluster 1, H2ag], H1ST1H2A1 [histone cluster 1, H2ai], H1ST1H2AJ [histone cluster 1, H2aj], H1ST1H2AK [histone cluster 1, H2ak], H1STIH2AL [histone cluster 1, H2al], H1STIH2AM [histone cluster 1, H2 am], HISTIH3E [histone cluster 1, H3e], H1ST2H2AA3 [histone cluster 2, H2aa3], H1ST2H2AA4 [histone cluster 2, H2aa4], H1ST2H2AC [histone cluster 2, H2ac], HKR1 [GLI-Kruppel family member HKR1], HLA-A [major histocompatibility complex, class I, A], HLA-B [major histocompatibility complex, class I, B], HLA-C [major histocompatibility complex, class I, C], HLA-DMA [major histocompatibility complex, class II, DM alpha], HLA-DOB [major histocompatibility complex, class II, DO beta], HLA-DQA1 [major histocompatibility complex, class II, DQ alpha 1], HLA-DQB1 [major histocompatibility complex, class II, DQ beta 1], HLA-DRA [major histocompatibility complex, class II, DR alpha], HLA-DRB1 [major histocompatibility complex, class II, DR beta 1], HLA-DRB4 [major histocompatibility complex, class II, DR beta 4], HLA-DRB5 [major histocompatibility complex, class II, DR beta 5], HLA-E [major histocompatibility complex, class I, E], HLA-F [major histocompatibility complex, class I, F], HLA-G [major histocompatibility complex, class I, G], HLCS [holocarboxylase synthetase (biotin-(proprionyl-Coenzyme A-carboxylase (ATP-hydrolysing)) ligase)], HMBS [hydroxymethylbilane synthase], HMGA1 [high mobility group AT-hook 1], HMGA2 [high mobility group AT-hook 2], HMGB1 [high-mobility group box 1], HMGCR [3-hydroxy-3-methylglutaryl-Coenzyme A reductase], HMGN1 [high-mobility group nucleosome binding domain 1], HMOX1 [heme oxygenase (decycling) 1], HMOX2 [heme oxygenase (decycling) 2], HNF1A [HNF1 homeobox A], HNF4A [hepatocyte nuclear factor 4, alpha], HNMT [histamine N-methyltransferase], HNRNPA2B1 [heterogeneous nuclear ribonucleoprotein A2/B1], HNRNPK [heterogeneous nuclear ribonucleoprotein K], HNRNPL [heterogeneous nuclear ribonucleoprotein L], HNRNPU [heterogeneous nuclear ribonucleoprotein U (scaffold attachment factor A)], HNRPDL [heterogeneous nuclear ribonucleoprotein D-like], HOMER1 [homer homolog 1 (Drosophila)], HOXA1 [homeobox A1], HOXA10 [homeobox A10], HOXA2 [homeobox A2], HOXAS [homeobox AS], HOXA9 [homeobox A9], HOXB1 [homeobox BI], HOXB4 [homeobox B4], HOXB9 [horneobox B9], HOXD11 [horneobox D11], HOXD12 [horneobox D12], HOXD13 [horneobox D13], HP [haptoglobin], HPD [4-hydroxyphenylpyruvate dioxygenase], HPRT1 [hypoxanthine phosphoribosyltransferase 1], HPS4 [Hermansky-Pudlak syndrome 4], HPX [hemopexin], HRAS [v-Ha-ras Harvey rat sarcoma viral oncogene homolog], HRG [histidine-rich glycoprotein], HRH1 [histamine receptor H1], HRH2 [histamine receptor H2], HRH3 [histamine receptor H3], HSD11B1 [hydroxysteroid (11-beta) dehydrogenase 1], HSD11B2 [hydroxysteroid (11-beta) dehydrogenase 2], HSD17B10 [hydroxysteroid (17-beta) dehydrogenase 10], HSD3B2 [hydroxy-delta-S-steroid dehydrogenase, 3 beta- and steroid delta-isomerase 2], HSF1 [heat shock transcription factor 1], HSP90AA1 [heat shock protein 90 kDa alpha (cytosolic), class A member 1], HSP90B1 [heat shock protein 90 kDa beta (Grp94), member 1], HSPA1A [heat shock 70 kDa protein 1A], HSPA4 [heat shock 70 kDa protein 4], HSPAS [heat shock 70 kDa protein S (glucose-regulated protein, 7f\ kDa)], HSPAR [heat shock 70 kDa protein R], HSPA9 [heat shock 70 kDa protein 9 (mortalin)], HSPB1 [heat shock 27 kDa protein 1], HSPD 1 [heat shock 60 kDa protein 1 (chaperonin)], HSPE1 [heat shock 10 kDa protein 1 (chaperonin 10)], HSPG2 [heparan sulfate proteoglycan 2], HTN1 [histatin 1], HTR1A [S-hydroxytryptamine (serotonin) receptor 1A], HTR1B [S-hydroxytryptamine (serotonin) receptor IB], HTRID [S-hydroxytryptamine (serotonin) receptor ID], HTRIE [S-hydroxytryptamine (serotonin) receptor IE], HTR1F [S-hydroxytryptamine (serotonin) receptor IF], HTR2A [S-hydroxytryptamine (serotonin) receptor 2A], HTR2B [S-hydroxytryptamine (serotonin) receptor 2B], HTR2c [S-hydroxytryptamine (serotonin) receptor 20], HTR3A [S-hydroxytryptamine (serotonin) receptor 3A], HTR3B [S-hydroxytryptamine (serotonin) receptor 3B], HTRSA [S-hydroxytryptamine (serotonin) receptor SA], HTR6 [S-hydroxytryptamine (serotonin) receptor 6], HTR7 [S-hydroxytryptamine (serotonin) receptor 7 (adenylate cyclase-coupled)], HTT [huntingtin], HYAL1 [hyaluronoglucosaminidase 1], HYOU1 [hypoxia up-regulated 1], 1APP [islet amyloid polypeptide], IBSP [integrin-binding sialoprotein], ICAM1 [intercellular adhesion molecule 1], ICAM2 [intercellular adhesion molecule 2], ICAM3 [intercellular adhesion molecule 3], ICAMS [intercellular adhesion moleculeS, telencephalin], ICOS [inducible T-cell co-stimulator], ID1 [inhibitor of DNA binding 1, dominant negative helix-loop-helix protein], ID2 [inhibitor of DNA binding 2, dominant negative helix-loop-helix protein], ID3 [inhibitor of DNA binding 3, dominant negative helix-loop-helix protein], ID4 [inhibitor of DNA binding 4, dominant negative helix-loop-helix protein], IDE [insulin-degrading enzyme], IDi1 [isopentenyl-diphosphate delta isomerase 1], IDO1 [indoleamine 2 [3-dioxygenase 1], IDS [iduronate 2-sulfatase], IDUA [iduronidase, alpha-L-], IER3 [immediate early response 3], IF127 [interferon, alpha-inducible protein 27], IFNa1 [interferon, alpha 1], IFNa2 [interferon, alpha 2], IFNAR1 [interferon (alpha, beta and omega) receptor 1], IFNAR2 [interferon (alpha, beta and omega) receptor 2], IFNB1 [interferon, beta 1, fibroblast], IFNG [interferon, gamma], IFNGR1 [interferon gamma receptor 1], IFNGR2 [interferon gamma receptor 2 (interferon gamma transducer 1)], IGF1 [insulin-like growth factor 1 (somatomedin C)], IGF1R [insulin-like growth factor 1 receptor], IGF2 [insulin-like growth factor 2 (somatomedin A)], IGF2R [insulin-like growth factor 2 receptor], IGFBP1 [insulin-like growth factor binding protein 1], 1GFBP2 [insulin-like growth factor binding protein 2, 36 kDa], TGFBP3 [insulin-like growth factor binding protein 3], TGFBP4 [insulin-like growth factor binding protein 4], IGFBP5 [insulin-like growth factor binding protein 5], IGFBP6 [insulin-like growth factor binding protein 6], IGFBP7 [insulin-like growth factor binding protein 7], IGHA1 [immunoglobulin heavy constant alpha 1], IGHE [immunoglobulin heavy constant epsilon], IGHG1 [immunoglobulin heavy constant gamma 1 (G1m marker)], IGHJ1 [immunoglobulin heavy joining 1], IGHM [immunoglobulin heavy constant mu], IGHMBP2 [immunoglobulin mu binding protein 2], TGKC [immunoglobulin kappa constant], TKBKAP [inhibitor of kappa light polypeptide gene enhancer in B-cells, kinase complex-associated protein], IKBKB [inhibitor of kappa light polypeptide gene enhancer in B-cells, kinase beta], IKZF1 [IKAROS family zinc finger 1 (Ikaros)], IL10O [interleukin 10], IL1 ORA [interleukin 10 receptor, alpha], IL1 ORB [interleukin 10 receptor, beta], IL11 [interleukin 11], IL11RA [interleukin 11 receptor, alpha], IL12A [interleukin 12A (natural killer cell stimulatory factor 1, cytotoxic lymphocyte maturation factor 1, p35)], IL12B [interleukin 12B (natural killer cell stimulatory factor 2, cytotoxic lymphocyte maturation factor 2, p40)], IL12RB1 [interleukin 12 receptor, beta 1], IL13 [interleukin 13], IL1S [interleukin 15], IL15RA [interleukin 15 receptor, alpha], IL16 [interleukin 16 (lymphocyte chemoattractant factor)], IL17A [interleukin 17A], IL18 [interleukin 18 (interferon-gamma-inducing factor)], IL18BP [interleukin 18 binding protein], ILIA [interleukin 1, alpha], IL1B [interleukin 1, beta], IL1F7 [interleukin 1 family, member 7 (zeta)], IL1R1 [interleukin 1 receptor, type I], IL1R2 [interleukin 1 receptor, type II], IL1RAPL1 [interleukin 1 receptor accessory protein-like 1], IL1RL1 [interleukin 1 receptor-like 1], IL1RN [interleukin 1 receptor antagonist], IL2 [interleukin 2], IL21 [interleukin 21], IL22 [interleukin 22], IL23A [interleukin 23, alpha subunit p19], IL23R [interleukin 23 receptor], IL29 [interleukin 29 (interferon, lambda 1)], IL2RA [interleukin 2 receptor, alpha], IL2RB [interleukin 2 receptor, beta], IL3 [interleukin 3 (colony-stimulating factor, multiple)], IL3RA [interleukin 3 receptor, alpha (low affinity)], IL4 [interleukin 4], IL4R [interleukin 4 receptor], IL5 [interleukin 5 (colony-stimulating factor, eosinophil)], IL6 [interleukin 6 (interferon, beta 2)], IL6R [interleukin 6 receptor], IL6ST [interleukin 6 signal transducer (gp130, oncostatin M receptor)], IL7 [interleukin 7], IL7R [interleukin 7 receptor], IL8 [interleukin 8], IL9 [interleukin 9], ILK [integrin-linked kinase], IMMP2L [IMP2 inner mitochondrial membrane peptidase-like (S. cerevisiae)], IMMT [inner membrane protein, mitochondrial (mitofilin)], IMPA1 [inositol(myo)-1(or 4)-monophosphatase 1], IMPDH2 [IMP (inosine monophosphate) dehydrogenase 2], INADL [InaD-like (Drosophila)], INCENP [inner centromere protein antigens 135/155 kDa], ING1 [inhibitor of growth family, member 1], ING3 [inhibitor of gro″'ih family, member 3], INHA [inhibin, alpha], INHBA [inhibin, beta A], INPP1 [inositol polyphosphate-1-phosphatase], INPP5D [inositol polyphosphate-5-phosphatase, 145 kDa], INPP5E [inositol polyphosphate-5-phosphatase, 72 kDa], INPP5J [inositol polyphosphate-5-phosphatase J], INPPL1 [inositol polyphosphate phosphatase-like 1], INS [insulin], INSIG2 [insulin induced gene 2], INS-IGF2 [INS-IGF2 readthrough transcript], INSL3 [insulin-like 3 (Leydig cell)], INSR [insulin receptor], INVS [inversin], IQCB1 [IQ motif containing B1], IQGAP1 [IQ motif containing GTPase activating protein 1], IRAK1 [interleukin-1 receptor-associated kinase 1], IRAK4 [interleukin-1 receptor-associated kinase 4], 1REB2 [iron-responsive element binding protein 2], 1RF1 [interferon regulatory factor 1], TRF4 [interferon regulatory factor 4], TRF8 [interferon regulatory factor 8], IRS1 [insulin receptor substrate 1], IRS2 [insulin receptor substrate 2], IRS4 [insulin receptor substrate 4], IRX3 [iroquois homeobox 3], ISG15 [ISG15 ubiquitin-like modifier], ISL1 [ISL LIM homeobox 1], ISL2 [ISL LIM homeobox 2], ISLR2 [immunoglobulin superfamily containing leucine-rich repeat 2], ITGA2 [integrin, alpha 2 (CD49B, alpha 2 subunit of VLA-2 receptor)], ITGA2B [integrin, alpha 2b (platelet glycoprotein TTb of TTb/TTTa complex, antigen CD41)], TTGA3 [integrin, alpha 3 (antigen CD49C, alpha 3 subunit of VLA-3 receptor)], ITGA4 [integrin, alpha 4 (antigen CD49D, alpha 4 subunit of VLA-4 receptor)], ITGA5 [integrin, alpha 5 (fibronectin receptor, alpha polypeptide)], ITGA6 [integrin, alpha 6], ITGA9 [integrin, alpha 9], ITGAL [integrin, alpha L (antigen CD 11A (p180), lymphocyte function-associated antigen 1; alpha polypeptide)], ITGAM [integrin, alpha M (complement component 3 receptor 3 subunit)], ITGAV [integrin, alpha V (vitronectin receptor, alpha polypeptide, antigen CD51)], ITGAX [integrin, alpha X (complement component 3 receptor 4 subunit)], ITGB1 [integrin, beta 1 (fibronectin receptor, beta polypeptide, antigen CD29 includes MDF2, MSK12)], ITGB2 [integrin, beta 2 (complement component 3 receptor 3 and 4 subunit)], ITGB3 [integrin, beta 3 (platelet glycoprotein Ilia, antigen CD61)], ITGB4 [integrin, beta 4], ITGB6 [integrin, beta 6], ITGB7 [integrin, beta 7], ITIH4 [inter-alpha (globulin) inhibitor H4 (plasma Kallikrein-sensitive glycoprotein)], ITM2B [integral membrane protein 2B], ITPR1 [inositol I [4 [5-triphosphate receptor, type 1], ITPR2 [inositol I [4 [5-triphosphate receptor, type 2], ITPR3 [inositol I [4 [5-triphosphate receptor, type 3], ITSN1 [intersectin 1 (SH3 domain protein)], ITSN2 [intersectin 2], NL [involucrin], JAG1 bagged 1 (Alagille yndrome)], JAK1 [Janus kinase 1], JAK2 [Janus kinase 2], JAK3 [Janus kinase 3], JAM2 [junctional adhesion molecule 2], JARID2 [jumonji, AT rich interactive domain 2], JMJD1 C [jumonji domain containing 10], JMY [junction mediating and regulatory protein, p53 cofactor], JRKL [jerky homolog-like (mouse)], JUN [jun oncogene], JUNB [jun B proto-oncogene], JUND [jun D proto-oncogene], JUP [junction plakoglobin], KAL1 [Kallmann syndrome 1 sequence], KALRN [kalirin, RhoGEF kinase], KARS [lysyl-tRNA syntheta e], KAT2B [K(lysine) acetyltransferase 2B], KATNA1 [katanin p60 (ATPase-containing) subunit A 1], KATNB1 [katanin p80 (WD repeat containing) subunit B], KCNA4 [potassium voltage-gated channel, shaker-related subfamily, member 4], KCND1 [potassium voltage-gated channel, Shal-related subfamily, member 1], KCND2 [potassium voltage-gated channel, Shal-related subfamily, member 2], KCNE1 [potassium voltage-gated channel, Isk-related family, member 1], KCNE2 [potassium voltage-gated channel, Isk-related family, member 2], KCNH2 [potassium voltage-gated channel, subfamily H (eag-related), member 2], KCNH4 [potassium voltage-gated channel, subfamily H (eag-related), member 4], KCNJ15 [potassium inwardly-rectifying channel, subfamily J, member 15], KCNJ3 [potassium inwardly-rectifying channel, subfamily J, member 3], KCNJ4 [potassium inwardly-rectifying channel, subfamily J, member 4], KCNJ5 [potassium inwardly-rectifying channel, subfamily J, member 5], KCNJ6 [potassium inwardly-rectifying channel, subfamily J, member 6], KCNMA1 [potassium large conductance calcium-activated channel, subfamily M, alpha member 1], KCNN1 [potassium intermediate/small conductance calcium-activated channel, subfamily N, member 1], KCNN2 [potassium intermediate/small conductance calcium-activated channel, subfamily N, member 2], KCNN3 [potassium intermediate/small conductance calcium-activated channel, subfamily N, member 3], KCNQ1 [potassium voltage-gated channel, KQT-like subfamily, member 1], KCNQ2 [potassium voltage-gated channel, KQT-like subfamily, member 2], KDM5C [lysine (K)-specific demethylase 5C], KDR [kinase insert domain receptor (a type III receptor tyrosine kinase)], KIAA0101 [KIAA0101], KIAA0319 [KIAA0319], KIAA1715 [KTAA1715], KTDTNS220 [kinase D-interacting substrate, 220 kDa], KTF15 [kinesin family member 15], KIF16B [kinesin family member 16B], KIF IA [kinesin family member 1A], KIF2A [kinesin heavy chain member 2A], KIF2B [kinesin family member 2B], KIF3A [kinesin family member 3A], KIF5C [kinesin family member 5C], KIF7 [kinesin family member 7], KIR2DL1 [killer cell immunoglobulin-like receptor, two domains, long cytoplasmic tail, 1], KIR2DL3 [killer cell immunoglobulin-like receptor, two domains, long cytoplasmic tail, 3], KIR2DS2 [killer cell immunoglobulin-like receptor, two domains, short cytoplasmic tail, 2], KIR3DL1 [killer cell immunoglobulin-like receptor, three domains, long cytoplasmic tail, 1], KIR3DL2 [killer cell immunoglobulin-like receptor, three domains, long cytoplasmic tail, 2], KIRREL3 [kin ofiRRE like 3 (Drosophila)], KISS1 [KiSS-1 metastasis-suppressor], KISS1R [KISS1 receptor], KIT [v-kit Hardy-Zuckerman 4 feline sarcoma viral oncogene homolog], KITLG [KIT ligand], KL [klotho], KLF7 [Kruppel-like factor 7 (ubiquitous)], KLK1 [kallikrein 1], KLK10 [kallikrein-related peptidase 10], KLK11 [kallikrein-related peptidase 11], KLK2 [kallikrein-related peptidase 2], KLK3 [kallikrein-related peptidase 3], KLK5 [kallikrein-related peptidase 5], KLRD1 [killer cell lectin-like receptor subfamily D, member 1], KLRK1 [killer cell lectin-like receptor subfamily K, member 1], KMO [kynurenine 3-monooxygenase (kynurenine 3-hydroxylase)], KNG1 [kininogen 1], KPNA2 [karyopherin alpha 2 (RAG cohort 1, importin alpha 1)], KPNB1 [karyopherin (importin) beta 1], KPTN [kaptin (actin binding protein)], KRAS [v-Ki-ras2 Kirsten rat sarcoma viral oncogene homolog], KRIT1 [KRIT1, ankyrin repeat containing], KRT1 [keratin 1], KRT10 [keratin 10], KRT14 [keratin 14], KRT18 [keratin 18], KRT19 [keratin 19], KRT3 [keratin 3], KRT5 [keratin 5], KRT7 [keratin 7], KRT8 [keratin 8], KRTAP19-3 [keratin associated protein 19-3], KRTAP2-1 [keratin associated protein 2-1], L1CAM [L1 cell adhesion molecule], LACTB [lactamase, beta], LALBA [lactalbumin, alpha-], LAMAI [laminin, alpha 1], LAMB1 [laminin, beta 1], LAMB2 [laminin, beta 2 (laminin S)], LAMB4 [laminin, beta 4], LAMP1 [lysosomal-associated membrane protein 1], LAMP2 [lysosomal-associated membrane protein 2], LAP3 [leucine aminopeptidase 3], LAPTM4A [lysosomal protein transmembrane 4 alpha], LARGE [like-glycosyltransferase], LARS [leucyl-tRNA synthetase], LASP1 [LIM and SH3 protein 1], LAT2 [linker for activation off cells family, member 2], LBP [lipopolysaccharide binding protein], LBR [lamin B receptor], LCA10 [lung carcinoma-associated protein 10], LCA5 [Leber congenital amaurosis 5], LCAT [lecithin-cholesterol acyltransferase], LCK [lymphocyte-specific protein tyrosine kinase], LCN1 [lipocalin 1 (tear prealbumin)], LCN2 [lipocalin 2], LCP1 [lymphocyte cytosolic protein 1 (L-plastin)], LCP2 [lymphocyte cytosolic protein 2 (SH2 domain containing leukocyte protein of 76 kDa)], LCT [lactase], LOBI [LIM domain binding 1], LDB2 [LIM domain binding 2], LDHA [lactate dehydrogenase A], LDLR [low density lipoprotein receptor], LDLRAP1 [low density lipoprotein receptor adaptor protein 1], LEF1 [lymphoid enhancer-binding factor 1], LEO1 [Leo1, Pafl/RNA polymerase TT complex component, homolog (S. cerevisiae)], LEP [leptin], LEPR [leptin receptor], LGALS13 [lectin, galactoside-binding, soluble, 13], LGALS3 [lectin, galactoside-binding, soluble, 3], LGMN [legumain], LGR4 [leucine-rich repeat-containing G protein-coupled receptor 4], LGTN [ligatin], LHCGR [luteinizing hormone/choriogonadotropin receptor], LHFPL3 [lipoma HMGIC fusion partner-like 3], LHX1 [LIM homeobox 1], LHX2 [LTM homeobox 2], LHX3 [LTM homeobox 3], LHX4 [LTM homeobox 4], LHX9 [LTM homeobox 9], LIF [leukemia inhibitory factor (cholinergic differentiation factor)], LIFR [leukemia inhibitory factor receptor alpha], LIG1 [ligase I, DNA, ATP-dependent], LIG3 [ligase III, DNA, ATP-dependent], LIG4 [ligase N, DNA, ATP-dependent], LILRA3 [leukocyte immunoglobulin-like receptor, subfamily A (without TM domain), member 3], LILRB1 [leukocyte immunoglobulin-like receptor, subfamily B (with TM and ITIM domains), member 1], LIMK1 [LIM domain kinase 1], LIMK2 [LIM domain kinase 2], LIN7A [lin-7 homolog A (C. elegans)], LIN7B [lin-7 homolog B (C. elegans)], LIN7C [lin-7 homolog C (C. elegans)], LING01 [leucine rich repeat and Ig domain containing 1], LIPC [lipase, hepatic], LIPE [lipase, hormone-sensitive], LLGL1 [lethal giant larvae homolog 1 (Drosophila)], LMAN1 [lectin, mannose-binding, 1], LMNA [lamin A/C], LM02 [LIM domain only 2 (rhombotin-like 1)], LMXIA [LIM homeobox transcription factor 1, alpha], LMX1B [LIM homeobox transcription factor 1, beta], LNPEP [leucyl!cystinyl aminopeptidase], LOC400590 [hypothetical LOC400590], LOC646021 [similar to hCG1774990], LOC646030 [similar to hCG1991475], LOC646627 [phospholipase inhibitor], LOR [loricrin], LOX [lysyl oxidase], LOXL1 [lysyl oxidase-like 1], LPA [lipoprotein, Lp(a)], LPL [lipoprotein lipase], LPO [lactoperoxidase], LPP [LIM domain containing preferred translocation partner in lipoma], LPPR1 [lipid phosphate phosphatase-related protein type 1], LPPR3 [lipid phosphate phosphatase-related protein type 3], LPPR4 [lipid phosphate phosphatase-related protein type 4], LPXN [leupaxin], LRP1 [low density lipoprotein receptor-related protein 1], LRP6 [low density lipoprotein receptor-related protein 6], LRP8 [low density lipoprotein receptor-related protein 8, apolipoprotein e receptor], LRPAP1 [low density lipoprotein receptor-related protein associated protein 1], LRPPRC [leucine-rich PPR-motif containing], LRRC37B [leucine rich repeat containing 37B], LRRC4C [leucine rich repeat containing 40], LRRTM1 [leucine rich repeat transmembrane neuronal I], LSAMP [limbic system-associated membrane protein], LSM2 [LSM2 homolog, U6 small nuclear RNA associated (S. cerevisiae)], LSS [lanosterol synthase (2 [3-oxidosqualene-lanosterol cyclase)], LTA [lymphotoxin alpha (TNF superfamily, member 1)], LTA4H [leukotriene A4 hydrolase], LTBP1 [latent transforming growth factor beta binding protein 1], LTBP4 [latent transforming growth factor beta binding protein 4], LTBR [lymphotoxin beta receptor (TNFR superfamily, member 3)], LTC4S [leukotriene C4 synthase], LTF [lactotransferrin], LY96 [lymphocyte antigen 96], LYN [v-yes-1 Yamaguchi sarcoma viral related oncogene homolog], LYVE1 [lymphatic vessel endothelial hyaluronan receptor 1], M6PR [mannose-6-phosphate receptor (cation dependent)], MAB21L1 [mab-21-like 1 (C. elegans)], MAB21L2 [mab-2′-like 2 (C. elegans)], MAF [v-mafmusculoaponeurotic fibrosarcoma oncogene homolog (avian)], MAG [myelin associated glycoprotein], MAGEA1 [melanoma antigen family A, 1 (directs expression of antigen MZ2-E)], MAGEL2 [MAGE-like 2], MAL [mal, T-cell differentiation protein], MAML2 [mastermind-like 2 (Drosophila)], MAN2A1 [mannosidase, alpha, class 2A, member 1], MANBA [mannosidase, beta A, lysosomal], MANF [mesencephalic astrocyte-derived neurotrophic factor], MAOA [monoamine oxidase A], MAOB [monoamine oxidase B], MAP1B [microtubule-associated protein IB], MAP2 [microtubule-associated protein 2], MAP2K1 [mitogen-activated protein kinase kinase 1], MAP2K2 [mitogen-activated protein kinase kinase 2], MAP2K3 [mitogen-activated protein kinase kinase 3], MAP2K4 [mitogen-activated protein kinase kinase 4], MAP3K1 [mitogen-activated protein kinase kinase kinase 1], MAP3K12 [mitogen-activated protein kinase kinase kinase 12], MAP3K13 [mitogen-activated protein kinase kinase kinase 13], MAP3K14 [mitogen-activated protein kinase kinase kinase 14], MAP3K4 [mitogen-activated protein kinase kinase kinase 4], MAP3K7 [mitogen-activated protein kinase kinase kinase 7], MAPK1 [mitogen-activated protein kinase 1], MAPK10 [mitogen-activated protein kinase 10], MAPK14 [mitogen-activated protein kinase 14], MAPK3 [mitogen-activated protein kinase 3], MAPK8 [mitogen-activated protein kinase 8], MAPK81P2 [mitogen-activated protein kinase 8 interacting protein 2], MAPK81P3 [mitogen-activated protein kinase 8 interacting protein 3], MAPK9 [mitogen-activated protein kinase 9], MAPKAPK2 [mitogen-activated protein kinase-activated protein kinase 2], MAPKSPI [MAPK scaffold protein 1], MAPRE3 [microtubule-associated protein, RP/EB family, member 3], MAPT [microtubule-associated protein tau], MARCKS [myristoylated alanine-rich protein kinase C substrate], MARK1 [MAP/microtubule affinity-regulating kinase 1], MARK2 [MAP/microtubule affinity-regulating kinase 2], MAT2A [methionine adenosyltransferase II, alpha], MATR3 [matrin 3], MAX [MYC associated factor X], MAZ [MYC-associated zinc finger protein (purine-binding transcription factor)], MB [myoglobin], MBD1 [methyl-CpG binding domain protein 1], MBD2 [methyl-CpG binding domain protein 2], MBD3 [methyl-CpG binding domain protein 3], MBD4 [methyl-CpG binding domain protein 4], MBL2 [mannose-binding lectin (protein C) 2, soluble (opsonic defect)], MBP [myelin basic protein], MBTPS1 [membrane-bound transcription factor peptidase, site 1], MC1R [melanocortin 1 receptor (alpha melanocyte stimulating hormone receptor)], MC3R [melanocortin 3 receptor], MC4R [melanocortin 4 receptor], MCCC2 [methylcrotonoyl-Coenzyme A carboxylase 2 (beta)], MCF2L [MCF.2 cell line derived transforming sequence-like], MCHR1 [melanin-concentrating hormone receptor 1], MCL1 [myeloid cell leukemia sequence 1 (BCL2-related)], MCM7 [minichromosome maintenance complex component 7], MCPH1 [microcephalin 1], MDC1 [mediator of DNA-damage checkpoint 1], MDFIC [MyoD family inhibitor domain containing], MDGA1 [MAM domain containing glycosylphosphatidylinositol anchor 1], MDK [midkine (neurite growth-promoting factor 2)], MDM2 [Mdm2 p53 binding protein homolog (mouse)], ME2 [malic enzyme 2, NAD(+)-dependent, mitochondrial], MECP2 [methyl CpG binding protein 2 (Rett syndrome)], MED1 [mediator complex subunit 1], MED12 [mediator complex subunit 12], MED24 [mediator complex subunit 24], MEF2A [myocyte enhancer factor 2A], MEF2C [myocyte enhancer factor 20], MEISI [Meis homeobox 1], MEN1 [multiple endocrine neoplasia 1], MERTK [c-mer proto-oncogene tyrosine kinase], MESP2 [mesoderm posterior 2 homolog (mouse)], MEST [mesoderm specific transcript homolog (mouse)], MET [met proto-oncogene (hepatocyte growth factor receptor)], METAP2 [methionyl aminopeptidase 2], METRN [meteorin, glial cell differentiation regulator], MFSD6 [major facilitator superfamily domain containing 6], MGAT2 [mannosyl (alpha-1 [6-)-glycoprotein beta-1 [2-N-acetylglucosaminyltransferase], MGMT [0-6-methylguanine-DNA methyltransferase], MGP [matrix Gla protein], MGST1 [microsomal glutathione S-transferase 1], MICA [MHC class I polypeptide-related sequence A], MICAL1 [microtubule associated monoxygenase, calponin and LTM domain containing 1], MICB [MHC class T polypeptide-related sequence B], MIF [macrophage migration inhibitory factor (glycosylation-inhibiting factor)], MITF [microphthalmia-associated transcription factor], MK167 [antigen identified by monoclonal antibody Ki-67], MKKS [McKusick-Kaufman syndrome], MKNKI [MAP kinase interacting serine/threonine kinase 1], MKRN3 [makorin ring finger protein 3], MKS1 [Meckel syndrome, type 1], MLH1 [mutL homolog 1, colon cancer, nonpolyposis type 2 (E. coli)], MLL [myeloid/lymphoid or mixed-lineage leukemia (trithorax homolog, Drosophila)], MLLT4 [myeloid/lymphoid or mixed-lineage leukemia (trithorax homolog, Drosophila); translocated to, 4], MLPH [melanophilin], MLX [MAX-like protein X], MLXIPL [MLX interacting protein-like], MME [membrane metallo-endopeptidase], MMP1 [matrix metallopeptidase 1 (interstitial collagenase)], MMP 10 [matrix metallopeptidase 10 (stromelysin 2)], MMP12 [matrix metallopeptidase 12 (macrophage elastase)], MMP13 [matrix metallopeptidase 13 (collagenase 3)], MMP14 [matrix metallopeptidase 14 (membrane-inserted)], MMP2 [matrix metallopeptidase 2 (gelatinase A, 72 kDa gelatinase, 72 kDa type IV collagenase)], MMP24 [matrix metallopeptidase 24 (membrane-inserted)], MMP26 [matrix metallopeptidase 26], MMP3 [matrix metallopeptidase 3 (stromelysin1, progelatinase)], MMP7 [matrix metallopeptidase 7 (matrilysin, uterine)], MMP8 [matrix metallopeptidase 8 (neutrophil collagenase)], MMP9 [matrix metallopeptidase 9 (gelatinase B, 92 kDa gelatinase, 92 kDa type IV collagenase)], MN1 [meningioma (disrupted in balanced translocation) 1], MNAT1 [menage a trois homolog 1, cyclin H assembly factor (Xenopus laevis)], MNX1 [motor neuron and pancreas homeobox 1], MOG [myelin oligodendrocyte glycoprotein], MPL [myeloproliferative leukemia virus oncogene], MPO [myeloperoxidase], MPP1 [membrane protein, palmitoylated 1, 55 kDa], MPZL1 [myelin protein zero-like 1], MR1 [major histocompatibility complex, class-related], MRAP [melanocortin 2 receptor accessory protein], MRAS [muscle RAS oncogene homolog], MRC1 [mannose receptor, C type 1], MRGPRX1 [MAS-related GPR, member X1], MS4A1 [membrane-spanning 4-domains, subfamily A, member 1], MSH2 [mutS homolog 2, colon cancer, nonpolyposis type 1 (E. coli)], MSH3 [mutS homolog 3 (E. coli)], MSI1 [musashi homolog 1 (Drosophila)], MSN [moesin], MSR1 [macrophage scavenger receptor 1], MSTN [myostatin], MSX1 [rnsh homeobox 1], MSX2 [msh homeobox 2], MT2A [metallothionein 2A], MT3 [metallothionein 3], MT-ATP6 [mitochondrially encoded ATP synthase 6], MT-001 [mitochondrially encoded cytochrome c oxidase I], MT-C02 [mitochondrially encoded cytochrome c oxidase II], MT-C03 [rnitochondrially encoded cytochrome c oxidase III], MTF1 [metal-regulatory transcription factor 1], MTHFD1 [methylenetetrahydrofolate dehydrogenase (NADP+ dependent) 1, methenyltetrahydrofolate cyclohydrolase, formyltetrahydrofolate synthetase], MTHFD1L [methylenetetrahydrofolate dehydrogenase (NADP+ dependent) 1-like], MTHFR [5 [10-methylenetetrahydrofolate reductase (NADPH)], MTL5 [metallothionein-like 5, testis-specific (tesmin)], MTMR14 [myotubularin related protein 14], MT-ND6 [mitochondrially encoded NADH dehydrogenase 6], MTNR1A [melatonin receptor 1A], MTNR1B [melatonin receptor IB], MTOR [mechanistic target of rapamycin (serine/threonine kinase)], MTR [5-methyltetrahydrofolate-homocysteine methyltransferase], MTRR [5-methyltetrahydrofolate-homocysteine methyltransferase reductase], MTTP [microsomal triglyceride transfer protein], MUC 1 [mucin 1, cell surface associated], MUCI6 [mucin 16, cell surface associated], MUC19 [mucin 19, oligomeric], MUC2 [mucin 2, oligomeric mucus/gel-forming], MUC3A [mucin 3A, cell surface associated], MUC5AC [mucin 5AC, oligomeric mucus/gel-forming], MUSK [muscle, skeletal, receptor tyrosine kinase], MUT [methylmalonyl Coenzyme A mutase], MVK [mevalonate kinase], MVP [major vault protein], MX1 [myxovirus (influenza virus) resistance 1, interferon-inducible protein p78 (mouse)], MXD1 [MAX dimerization protein 1], MXI1 [MAX interactor 1], MYB [v-myb myeloblastosis viral oncogene homolog (avian)], MYC [v-myc myelocytomatosis viral oncogene homolog (avian)], MYCBP2 [MYC binding protein 2], MYCN [v-myc myclocytomatosis viral related oncogene, neuroblastoma derived (avian)], MYD88 [myeloid differentiation primary response gene (88)], MYF5 [myogenic factor 5], MYH10 [myosin, heavy chain 10, non-muscle], MYH14 [myosin, heavy chain 14, non-muscle], MYH7 [myosin, heavy chain 7, cardiac muscle, beta], MYL1 [myosin, light chain 1, alkali; skeletal, fast], MYL10 [myosin, light chain 10, regulatory], MYL12A [myosin, light chain 12A, regulatory, non-sarcomeric], MYL12B [myosin, light chain 12B, regulatory], MYL2 [myosin, light chain 2, regulatory, cardiac, slow], MYL3 [myosin, light chain 3, alkali; ventricular, skeletal, slow], MYL4 [myosin, light chain 4, alkali; atrial, embryonic], MYL5 [myosin, light chain 5, regulatory], MYL6 [myosin, light chain 6, alkali, smooth muscle and non-muscle], MYL6B [myosin, light chain 6B, alkali, smooth muscle and non-muscle], MYL7 [myosin, light chain 7, regulatory], MYL9 [myosin, light chain 9, regulatory], MYLK [myosin light chain kinase], MYLPF [myosin light chain, phosphorylatable, fast skeletal muscle], MYO1D [myosin ID], MYOSA [myosin VA (heavy chain 12, myoxin)], MYOC [myocilin, trabecular meshwork inducible glucocorticoid response], MYOD1 [myogenic differentiation 1], MYOG [myogenin (myogenic factor 4)], MYOM2 [myomesin (M-protein) 2, 165 kDa], MYST3 [MYST histone acetyltransferase (monocytic leukemia) 3], NACA [nascent polypeptide-associated complex alpha subunit], NAGLU [N-acetylglucosaminidase, alpha-], NAIP [NLR family, apoptosis inhibitory protein], NAMPT [nicotinamide phosphoribosyltransferase], NANOG [Nanog homeobox], NANS [N-acetylneuraminic acid synthase], NAP1L2 [nucleosome assembly protein 1-like 2], NAPA [N-ethylmaleimide-sensitive factor attachment protein, alpha], NAPG [N-ethylmaleimide-sensitive factor attachment protein, gamma], NAT2 [N-acetyltransferase 2 (arylamine N-acetyltransferase)], NAV1 [neuron navigator 1], NAV3 [neuron navigator 3], NBEA [neurobeachin], NCALD [neurocalcin delta], NCAM1 [neural cell adhesion molecule 1], NCAM2 [neural cell adhesion molecule 2], NCF1 [neutrophil cytosolic factor 1], NCF2 [neutrophil cytosolic factor 2], NCK1 [NCK adaptor protein 1], NCK2 [NCK adaptor protein 2], NCKAP1 [NCK-associated protein 1], NCL [nucleolin], NCOA2 [nuclear receptor coactivator 2], NCOA3 [nuclear receptor coactivator 3], NCOR1 [nuclear receptor co-repressor 1], NCOR2 [nuclear receptor co-repressor 2], NDE1 [nudE nuclear distribution gene E homolog 1 (A. nidulans)], NDEL1 [nudE nuclear distribution gene E homolog (A. nidulans)-like 1], NDN [necdin homolog (mouse)], NDNL2 [necdin-like 2], NDP [Norrie disease (pseudoglioma)], NDUFA1 [NADH dehydrogenase (ubiquinone) 1 alpha subcomplex, 1, 7.5 kDa], NDUFAB1 [NADH dehydrogenase (ubiquinone) 1, alpha/beta subcomplex, 1, 8 kDa], NDUFS3 [NADH dehydrogenase (ubiquinone) Fe-S protein 3, 30 kDa (NADH-coenzyrne Q reductase)], NDUFV3 [NADH dehydrogenase (ubiquinone) flavoprotein 3, 10 kDa], NEDD4 [neural precursor cell expressed, developmentally down-regulated 4], NEDD4L [neural precursor cell expressed, developmentally down-regulated 4-like], NEFH [neurofilament, heavy polypeptide], NEFL [neurofilament, light polypeptide], NEFM [neurofilament, medium polypeptide], NENF [neuron derived neurotrophic factor], NEO1 [neogenin homolog 1 (chicken)], NES [nestin], NET1 [neuroepithelial cell transforming 1], NEU1 [sialidase 1 (lysosomal sialidase)], NEU3 [sialidase 3 (membrane sialidase)], NEUROD1 [neurogenic differentiation 1], NEUROD4 [neurogenic differentiation 4], NEUROG1 [neurogenin 1], NEUROG2 [neurogenin 2], NF1 [neurofibromin 1], NF2 [neurofibromin 2 (merlin)], NFASC [neurofascin homolog (chicken)], NFAT5 [nuclear factor of activated T-cells 5, tonicity-responsive], NFATC1 [nuclear factor of activated T-cells, cytoplasmic, calcineurin-dependent 1], NFATC2 [nuclear factor of activated T-cells, cytoplasmic, calcineurin-dependent 2], NFATC3 [nuclear factor of activated T-cells, cytoplasmic, calcineurin-dependent 3], NFATC4 [nuclear factor of activated T-cells, cytoplasmic, calcineurin-dependent 4], NFE2L2 [nuclear factor (erythroid-derived 2)-like 2], NFIC [nuclear factor I/C (CCAAT-binding transcription factor)], NFIL3 [nuclear factor, interleukin 3 regulated], NFKB1 [nuclear factor of kappa light polypeptide gene enhancer in B-cells 1], NFKB2 [nuclear factor of kappa light polypeptide gene enhancer in B-cells 2 (p49/p100)], NFKBIA [nuclear factor of kappa light polypeptide gene enhancer in B-cells inhibitor, alpha], NFKBIB [nuclear factor of kappa light polypeptide gene enhancer in B-cells inhibitor, beta], NFKBIL1 [nuclear factor of kappa light polypeptide gene enhancer in B-cells inhibitor-like 1], NFYA [nuclear transcription factorY, alpha], NFYB [nuclear tran cription factorY, beta], NGEF [neuronal guanine nucleotide exchange factor], NGF [nerve growth factor (beta polypeptide)], NGFR [nerve growth factor receptor (TNFR superfamily, member 16)], NGFRAP1 [nerve growth factor receptor (TNFRSF16) associated protein 1], NHLRC1 [NHL repeat containing 1], NINJ1 [ninjurin 1], NINJ2 [ninjurin 2], NIP7 [nuclear import 7 homolog (S. cerevisiae)], NIPA1 [non imprinted in Prader-Willi/Angelman syndrome 1], NIPA2 [non imprinted in Prader-Willi/Angelman syndrome 2], NIPAL1 [NIPA-like domain containing 1], NIPAL4 [NIPA-like domain containing 4], NIPSNAP1 [nipsnap homolog 1 (C. elegans)], NISCH [nischarin], NIT2 [nitrilase family, member 2], NKX2-1 [NK2 homeobox 1], NKX2-2 [NK2 homeobox 2], NLGN1 [neuroligin 1], NLGN2 [neuroligin 2], NLGN3 [neuroligin 3], NLGN4X [neuroligin 4, X-linked], NLGN4Y [neuroligin 4, Y-linked], NLRP3 [NLR family, pyrin domain containing 3], NMB [neuromedin B], NME1 [non-metastatic cells 1, protein (NM23A) expressed in], NME2 [non-metastatic cells 2, protein (NM23B) expressed in], NME4 [non-metastatic cells 4, protein expressed in], NNAT [neuronatin], NOD1 [nucleotide-binding oligomerization domain containing 1], NOD2 [nucleotide-binding oligomerization domain containing 2], NOG [noggin], NOL6 [nucleolar protein family 6 (RNA-associated)], NOS1 [nitric oxide synthase 1 (neuronal)], NOS2 [nitric oxide synthase 2, inducible], NOS3 [nitric oxide synthase 3 (endothelial cell)], NOSTRIN [nitric oxide synthase trafficker], NOTCH1 [Notch homolog 1, translocation-associated (Drosophila)], NOTCH2 [Notch homolog 2 (Drosophila)], NOTCH3 [Notch homolog 3 (Drosophila)], NOV [nephroblastoma overexpressed gene], NOVA1 [neuro-oncological ventral antigen 1], NOVA2 [neuro-oncological ventral antigen 2], NOX4 [NADPH oxidase 4], NPAS4 [neuronal PAS domain protein 4], NPFF [neuropeptide FF-amide peptide precursor], NPHP1 [nephronophthisis 1 (juvenile)], NPHP4 [nephronophthisis 4], NPHS1 [nephrosis 1, congenital, Finnish type (nephrin)], NPM1 [nucleophosmin (nucleolar phosphoprotein B23, numatrin)], NPPA [natriuretic peptide precursor A], NPPB [natriuretic peptide precursor B], NPPC [natriuretic peptide precursor C], NPR1 [natriuretic peptide receptor A/guanylate cyclase A (atrionatriuretic peptide receptor A)], NPR3 [natriuretic peptide receptor C/guanylate cyclase C (atrionatriuretic peptide receptor C)], NPRL2 [nitrogen permease regulator-like 2 (S. cerevisiae)], NPTX1 [neuronal pentraxin I], NPTX2 [neuronal pentraxin II], NPY [neuropeptide Y], NPY1R [neuropeptide Y receptor Y1], NPY2R [neuropeptide Y receptor Y2], NPY5R [neuropeptide Y receptor Y5], NQO1 [NAD(P)H dehydrogenase, quinone 1], NQO2 [NAD(P)H dehydrogenase, quinone 2], NROB1 [nuclear receptor subfamily 0, group B, member 1], NROB2 [nuclear receptor subfamily 0, group B, member 2], NR1H3 [nuclear receptor subfamily 1, group H, member 3], NR1H4 [nuclear receptor subfamily 1, group H, member 4], NR1I2 [nuclear receptor subfamily 1, group I, member 2], NR1I3 [nuclear receptor subfamily 1, group I, member 3], NR2C1 [nuclear receptor subfamily 2, group C, member 1], NR2C2 [nuclear receptor subfamily 2, group C, member 2], NR2E1 [nuclear receptor subfamily 2, group E, member 1], NR2F1 [nuclear receptor subfamily 2, group F, member 1], NR2F2 [nuclearreceptor subfamily 2, group F, member 2], NR3C1 [nuclear receptor subfamily 3, group C, member 1 (glucocorticoid receptor)], NR3C2 [nuclear receptor subfamily 3, group C, member 2], NR4A2 [nuclear receptor subfamily 4, group A, member 2], NR4A3 [nuclear receptor subfamily 4, group A, member 3], NR5A1 [nuclear receptor subfamily 5, group A, member 1], NR6A1 [nuclear receptor subfamily 6, group A, member 1], NRAS [neuroblastoma RAS viral (v-ras) oncogene homolog], NRCAM [neuronal cell adhesion molecule], NRD1 [nardilysin (N-arginine dibasic convertase)], NRF1 [nuclear respiratory factor 1], NRG1 [neuregulin 1], NRIP1 [nuclear receptor interacting protein 1], NRN1 [neuritin 1], NRP1 [neuropilin 1], NRP2 [neuropilin 2], NRSN1 [neurensin 1], NRTN [nerniurin], NRXN1 [neurexin 1], NRXN3 [neurexin 3], NSD1 [nuclear receptor binding SET domain protein 1], NSF [N-ethylmaleimide-sensitive factor], NSUN5 [NOP2/Sun domain family, member 5], NT5E [5′-mucleotidase, ecto (CD73)], NTF3 [neurotrophin 3], NTF4 [neurotrophin 4], NTHL1 [nth endonuclease III-like 1 (E. coli)], NTN1 [netrin 1], NTN3 [netrin 3], NTN4 [netrin 4], NTNG1 [netrin G1], NTRK1 [neurotrophic tyrosine kinase, receptor, type 1], NTRK2 [neurotrophic tyrosine kinase, receptor, type 2], NTRK3 [neurotrophic tyrosine kinase, receptor, type 3], NTS [neurotensin], NTSR1 [neurotensin receptor 1 (high affinity)], NUCB2 [nucleobindin 2], NUDC [nuclear distribution gene C homolog (A. nidulans)], NUDT6 [nudix (nucleoside diphosphate linked moiety X)-type motif 6], NUDT7 [nudix (nucleoside diphosphate linked moiety X)-type motif7], NUMB [numb homolog (Drosophila)], NUP98 [nucleoporin 98 kDa], NUPR1 [nuclear protein, transcriptional regulator, 1], NXF1 [nuclear RNA export factor 1], NXNL1 [nucleoredoxin-like 1], OAT [ornithine aminotransferase], OCA2 [oculocutaneous albinism II], OCLN [occludin], OCM [oncomodulin], ODC1 [ornithine decarboxylase 1], OFD1 [oral-facial-digital syndrome 1], OGDH [oxoglutarate (alpha-ketoglutarate) dehydrogenase (lipoamide)], OLA1 [Obg-like ATPase 1], OLIG1 [oligodendrocyte transcription factor 1], OLTG2 [oligodendrocyte lineage transcription factor 2], OLR1 [oxidized low density lipoprotein (lectin-like) receptor 1], OMG [oligodendrocyte myelin glycoprotein], OPHN1 [oligophrenin 1], OPN1SW [opsin 1 (cone pigments), short-wave-sensitive], OPRD1 [opioid receptor, delta 1], OPRK1 [opioid receptor, kappa 1], OPRL1 [opiate receptor-like 1], OPRM1 [opioid receptor, mu 1], OPTN [optineurin], OSBP [oxysterol binding protein], OSBPL10 [oxysterol binding protein-like 10], OSBPL6 [oxysterol binding protein-like 6], OSM [oncostatinM], OTC [ornithine carbamoyltransferase], OTX2 [orthodenticle homeobox 2], OXA1L [oxidase (cytochrome c) assembly 1-like], OXT [oxytocin, prepropeptide], OXTR [oxytocin receptor], P2RX7 [purinergic receptor P2X, ligand-gated ion channel, 7], P2RY1 [purinergic receptor P2Y, G-protein coupled, 1], P2RY12 [purinergic receptor P2Y, G-protein coupled, 12], P2RY2 [purinergic receptor P2Y, G-protein coupled, 2], P4HB [proly14-hydroxylase, beta polypeptide], PABPC1 [poly(A) binding protein, cytoplasmic 1], PADI4 [peptidyl arginine deiminase, type IV], PAEP [progestagen-associated endometrial protein], PAFAH1B1 [platelet-activating factor acetylhydrolase 1b, regulatory subunit 1 (45 kDa)], PAFAH1B2 [platelet-activating factor acetylhydrolase 1b, catalytic subunit 2 (30 kDa)], PAG1 [phosphoprotein associated with glycosphingolipid microdomains 1], PAH [phenylalanine hydroxylase], PAK1 [p21 protein (Cdc42/Rac)-activated kinase 1], PAK2 [p21 protein (Cdc42/Rac)-activated kinase 2], PAK3 [p21 protein (Cdc42/Rac)-activated kinase 3], PAK-4 [p21 protein (Cdc42/Rac)-activated kinase 4], PAK6 [p21 protein (Cdc42/Rac)-activated kinase 6], PAK7 [p21 protein (Cdc42/Rac)-activated kinase 7], PAPPA [pregnancy-associated plasma protein A, pappalysin 1], PAPPA2 [pappalysin 2], PARD6A [par-6 partitioning defective 6 homolog alpha (C. elegans)], PARG [poly (ADP-ribose) glycohydrolase], PARK2 [Parkinson disease (autosomal recessive, juvenile) 2, parkin], PARK7 [Parkinson disease (autosomal recessive, early onset) 7], PARN [poly(A)-specific ribonuclease (deadenylation nuclease)], PARP1 [poly (ADP-ribose) polymerase 1], PAWR [PRKC, apoptosis, WT1, regulator], PAX2 [paired box 2], PAX3 [paired box 3], PAX5 [paired box 5], PAX6 [paired box 6], PAX7 [paired box 7], PBX1 [pre-B-cellleukemia homeobox 1], PC [pyruvate carboxylase], PCDH10 [protocadherin 10], PCDH19 [protocadherin 19], PCDHA12 [protocadherin alpha 12], PCK2 [phosphoenolpyruvate carboxykinase 2 (mitochondrial)], POLO [piccolo (presynaptic cytomatrix protein)], PCM1 [pericentriolar material 1], PCMT1 [protein-L-isoaspartate (D-aspartate)O-methyltransferase], PCNA [proliferating cell nuclear antigen], PCNT [pericentrin], PCP4 [Purkinje cell protein 4], PCSK7 [proprotein convertase subtilisin/kexin type 7], PDCD1 [programmed cell death 1], PDE11A [phosphodiesterase 11A], PDE3B [phosphodiesterase 3B, cGMP-inhibited], PDE4A [phosphodiesterase 4A, cAMP-specific (phosphodiesterase E2 dunce homolog, Drosophila)], PDE4B [phosphodiesterase 4B, cAMP-specific (phosphodiesterase E4 dunce homolog, Drosophila)], PDE4D [phosphodiesterase 4D, cAMP-specific (phosphodiesterase E3 dunce homolog, Drosophila)], PDE5A [phosphodiesterase 5A, cGMP-specific], PDE8A [phosphodiesterase 8A], PDGFA [platelet-derived growth factor alpha polypeptide], PDGFB [platelet-derived growth factor beta polypeptide (simian sarcoma viral (v-sis) oncogene homolog)], PDGFC [platelet derived growth factor C], PDGFD [platelet derived growth factor D], PDGFRA [platelet-derived growth factor receptor, alpha polypeptide], PDGFRB [platelet-derived growth factor receptor, beta polypeptide], PDHA1 [pyruvate dehydrogenase (lipoamide) alpha 1], PDIA2 [protein disulfide isomerase family A, member 2], PDIA3 [protein disulfide isomerase family A, member 3], PDLIM1 [PDZ and LIM domain 1], PDLIM7 [PDZ and LIM domain 7 (enigma)], PDP1 [pyruvate dehyrogenase phosphatase catalytic subunit 1], PDPN [podoplanin], PDXK [pyridoxal (pyridoxine, vitamin B6) kinase], PDXP [pyridoxal (pyridoxine, vitamin B6) phosphatase], PDYN [prodynorphin], PDZK1 [PDZ domain containing 1], PEBP1 [phosphatidylethanolamine binding protein 1], PECAM1 [platelet/endothelial cell adhesion molecule], PENK [proenkephalin], PERI [period homolog 1 (Drosophila)], PER2 [period homolog 2 (Drosophila)], PEX13 [peroxisomal biogenesis factor 13], PEX2 [peroxisomal biogenesis factor 2], PEX5 [peroxisomal biogenesis factor 5], PEX7 [peroxisomal biogenesis factor 7], PF4 [platelet factor 4], PFAS [phosphoribosylformylglycinamidine synthase], PFKL [phosphofructokinase, liver], PFKM [phosphofructokinase, muscle], PFN1 [profilin 1], PFN2 [profilin 2], PFN3 [profilin 3], PFN4 [profiling family, member 4], PGAM2 [phosphoglycerate mutase 2 (muscle)], PGD [phosphogluconate dehydrogenase], PGF [placental growth factor], PGK1 [phosphoglycerate kinase 1], PGM1 [phosphoglucomutase 1], PGR [progesterone receptor], PHB [prohibitin], PHEX [phosphate regulating endopeptidase homolog, X-linked], PHF10 [PHD finger protein 10], PHF8 [PHD finger protein 8], PHGDH [phosphoglycerate dehydrogenase], PHKA2 [phosphorylase kinase, alpha 2 (liver)], PHLDA2 [pleckstrin homology-like domain, family A, member 2], PHOX2B [paired-like homeobox 2b], PHYH [phytanoyl-CoA 2-hydroxylase], PHYHIP [phytanoyl-CoA 2-hydroxylase interacting protein], PIAS1 [protein inhibitor of activated STAT, 1], PICALM [phosphatidylinositol binding clathrin assembly protein], P1GF [phosphatidylinositol glycan anchor biosynthesis, class F], PIGP [phosphatidylinositol glycan anchor biosynthesis, class P], PIK3C2A [phosphoinositide-3-kinase, class 2, alpha polypeptide], PIK3C2B [phosphoinositide-3-kinase, class 2, beta polypeptide], PIK3C2G [phosphoinositide-3-kinase, class 2, gamma polypeptide], PIK3C3 [phosphoinositide-3-kinase, class 3], PIK3CA [phosphoinositide-3-kinase, catalytic, alpha polypeptide], PIK3CB [phosphoinositide-3-kinase, catalytic, beta polypeptide], PIK3CD [phosphoinositide-3-kinase, catalytic, delta polypeptide], PIK3CG [phosphoinositide-3-kinase, catalytic, gamma polypeptide], PIK3R1 [phosphoinositide-3-kinase, regulatory subunit 1 (alpha)], PIK3R2 [phosphoinositide-3-kinase, regulatory subunit 2 (beta)], PIK3R3 [phosphoinositide-3-kinase, regulatory subunit 3 (gamma)], PIK3R4 [phosphoinositide-3-kinase, regulatory subunit 4], PIK3R5 [phosphoinositide-3-kinase, regulatory subunit 5], PINK1 [PTEN induced putative kinase 1], PITX1 [paired-like homeodomain 1], PITX2 [paired-like homeodomain 2], PITX3 [paired-like homeodomain 3], PKD1 [polycystic kidney disease 1 (autosomal dominant)], PKD2 [polycystic kidney disease 2 (autosomal dominant)], PKHD1 [polycystic kidney and hepatic disease 1 (autosomal recessive)], PKLR [pyruvate kinase, liver and RBC], PKN2 [protein kinase N2], PKNOX1 [PBX/knotted 1 homeobox 1], PL-5283 [PL-5283 protein], PLA2G10 [phospholipase A2, group X], PLA2G2A [phospholipase A2, group IIA (platelets, synovial fluid)], PLA2G4A [phospholipase A2, group IVA (cytosolic, calcium-dependent)], PLA2G6 [phospholipase A2, group VI (cytosolic, calcium-independent)], PLA2G7 [phospholipase A2, group VII (platelet-activating factor acetylhydrolase, plasma)], PLAC4 [placenta-specific 4], PLAG1 [pleiomorphic adenoma gene 1], PLAGL1 [pleiomorphic adenoma gene-like 1], PLAT [plasminogen activator, tissue], PLAU [plasminogen activator, urokinase], PLAUR [plasminogen activator, urokinase receptor], PLCB1 [phospholipase C, beta 1 (phosphoinositide-specific)], PLCB2 [phospholipase C, beta 2], PLCB3 [phospholipase C, beta 3 (phosphatidylinositol-specific)], PLCB4 [phospholipase C, beta 4], PLCG1 [phospholipase C, gamma 1], PLCG2 [phospholipase C, gamma 2 (phosphatidylinositol-specific)], PLCL1 [phospholipase C-like 1], PLD1 [phospholipase D1, phosphatidylcholine-specific], PLD2 [phospholipase D2], PLEK [pleckstrin], PLEKHH1 [pleckstrin homology domain containing, family H (with MyTH4 domain) member 1], PLG [plasminogen], PLIN1 [perilipin 1], PLK1 [polo-like kinase 1 (Drosophila)], PLOD1 [procollagen-lysine 1,2-oxoglutarate 5-dioxygenase 1], PLP1 [proteolipid protein 1], PLTP [phospholipid transfer protein], PLXNA1 [plexin A1], PLXNA2 [plexin A2], PLXNA3 [plexin A3], PLXNA4 [plexin A4], PLXNB1 [plexin B1], PLXNB2 [plexin B2], PLXNB3 [plexin B3], PLXNC1 [plexin C1], PLXND1 [plexin D1], PML [promyelocytic leukemia], PMP2 [peripheral myelin protein 2], PMP22 [peripheral myelin protein 22], PMS2 [PMS2 postmeiotic segregation increased 2 (S. cerevisiae)], PMVK [phosphomevalonate kinase], PNOC [prepronociceptin], PNP [purine nucleoside phosphorylase], PNPLA6 [patatin-like phospholipase domain containing 6], PNPO [pyridoxamine 5′-phosphate oxidase], POFUT2 [protein O-fucosyltransferase 2], POLB [polymerase (DNA directed), beta], POLR1C [polymerase (RNA) I polypeptide C, 30 kDa], POLR2A [polymerase (RNA) II (DNA directed) polypeptide A, 220 kDa], POLR3K [polymerase (RNA) III (DNA directed) polypeptide K, 12.3 kDa], POM121C [POM121 membrane glycoprotein C], POMC [proopiomelanocortin], POMGNT1 [protein O-linked mannose beta1 [2-N-acetylglucosaminyltransferase], POMT1 [protein-O-mannosyltransferase 1], PON1 [paraoxonase 1], PON2 [paraoxonase 2], POR [P450 (cytochrome) oxidoreductase], POSTN [periostin, osteoblast specific factor], POU1FI [POU class 1 homeobox 1], POU2F1 [POU class 2 homeobox 1], POU3F4 [POU class 3 homeobox 4], POU4F1 [POU class 4 homeobox 1], POU4F2 [POU class 4 homeobox 2], POU4F3 [POU class 4 homeobox 3], POU5F1 [POU class 5 homeobox 1], PPA1 [pyrophosphatase (inorganic) 1], PPARA [peroxisome proliferator-activated receptor alpha], PPARD [peroxisome proliferator-activated receptor delta], PPARG [peroxisome proliferator-activated receptor gamma], PPARGC1A [peroxisome proliferator-activated receptor gamma, coactivator 1 alpha], PPAT [phosphoribosyl pyrophosphate amidotransferase], PPBP [pro-platelet basic protein (chemokine (C-X-C motif) ligand 7)], PPFIA1 [protein tyrosine phosphatase, receptor type, f polypeptide (PTPRF), interacting protein (liprin), alpha 1], PPF1A2 [protein tyrosine phosphatase, receptor type, f polypeptide (PTPRF), interacting protein (liprin), alpha 2], PPFIA3 [protein tyrosine phosphatase, receptor type, f polypeptide (PTPRF), interacting protein (liprin), alpha 3], PPFIBP1 [PTPRF interacting protein, binding protein 1 (liprin beta 1)], PPIC [peptidylprolyl isomerase C (cyclophilin C)], PPIG [peptidylprolyl isomerase G (cyclophilin G)], PPP1R15A [protein phosphatase 1, regulatory (inhibitor) subunit 15A], PPP1R1B [protein phosphatase 1, regulatory (inhibitor) subunit IB], PPP1R9A [protein phosphatase 1, regulatory (inhibitor) subunit 9A], PPP1R9B [protein phosphatase 1, regulatory (inhibitor) subunit 9B], PPP2CA [protein phosphatase 2, catalytic subunit, alpha isozyme], PPP2R4 [protein phosphatase 2A activator, regulatory subunit 4], PPP3CA [protein phosphatase 3, catalytic subunit, alpha isozyme], PPP3CB [protein phosphatase 3, catalytic subunit, beta isozyme], PPP3CC [protein phosphatase 3, catalytic subunit, gamma isozyme], PPP3R1 [protein phosphatase 3, regulatory subunit B, alpha], PPP3R2 [protein phosphatase 3, regulatory subunit B, beta], PPP4C [protein phosphatase 4, catalytic subunit], PPY [pancreatic polypeptide], PQBP1 [polyglutamine binding protein 1], PRAM1 [PML-RARA regulated adaptor molecule 1], PRAME [preferentially expressed antigen in melanoma], PRDM1 [PR domain containing 1, with ZNF domain], PRDM15 [PR domain containing 15], PRDM2 [PR domain containing 2, with ZNF domain], PRDX1 [peroxiredoxin 1], PRDX2 [peroxiredoxin 2], PRDX3 [peroxiredoxin 3], PRDX4 [peroxiredoxin 4], PRDX6 [peroxiredoxin 6], PRF1 [perforin 1 (pore forming protein)], PRKAA1 [protein kinase, AMP-activated, alpha 1 catalytic subunit], PRKAA2 [protein kinase, AMP-activated, alpha 2 catalytic subunit], PRKAB1 [protein kinase, AMP-activated, beta 1 non-catalytic subunit], PRKACA [protein kinase, cAMP-dependent, catalytic, alpha], PRKACB [protein kinase, cAMP-dependent, catalytic, beta], PRKACG [protein kinase, cAMP-dependent, catalytic, gamma], PRKAG1 [protein kinase, AMP-activated, gamma 1 non-catalytic subunit], PRKAG2 [protein kinase, AMP-activated, gamma 2 non-catalytic subunit], PRKAR1A [protein kinase, cAMP-dependent, regulatory, type I, alpha (tissue specific extinguisher 1)], PRKAR1B [protein kinase, cAMP-dependent, regulatory, type I, beta], PRKAR2A [protein kinase, cAMP-dependent, regulatory, type II, alpha], PRKAR2B [protein kinase, cAMP-dependent, regulatory, type II, beta], PRKCA [protein kinase C, alpha], PRKCB [protein kinase C, beta], PRKCD [protein kinase C, delta], PRKCE [protein kinase C, epsilon], PRKCG [protein kinase C, gamma], PRKCH [protein kinase C, eta], PRKC1 [protein kinase C, iota], PRKCQ [protein kinase C, theta], PRKCZ [protein kinase C, zeta], PRKD1 [protein kinase D1], PRKDC [protein kinase, DNA-activated, catalytic polypeptide], PRKG1 [protein kinase, cGMP-dependent, type I], PRL [prolactin], PRLR [prolactin receptor], PRMT1 [protein arginine methyltransferase 1], PRNP [prion protein], PROC [protein C (inactivator of coagulation factors Va and VIIIa)], PROCR [protein C receptor, endothelial (EPCR)], PRODH [proline dehydrogenase (oxidase) 1], PROK1 [prokineticin 1], PROK2 [prokineticin 2], PROM1 [prominin 1], PR051 [protein S (alpha)], PRPF40A [PRP40 pre-mRNA processing factor 40 homolog A (S. cerevisiae)], PRPF40B [PRP40 pre-mRNA processing factor 40 homolog B (S. cerevisiae)], PRPH [peripherin], PRPH2 [peripherin 2 (retinal degeneration, slow)], PRPS1 [phosphoribosyl pyrophosphate synthetase 1], PRRG4 [proline rich Gla (G-carboxyglutamic acid) 4 (transmembrane)], PRSS8 [protease, serine, 8], PRTN3 [proteinase 3], PRX [periaxin], PSAP [prosaposin], PSEN1 [presenilin 1], PSEN2 [presenilin 2 (Alzheimer disease 4)], PSG1 [pregnancy specific beta-1-glycoprotein 1], PSTP1 [PC4 and SFRS1 interacting protein 1], PSMA5 [proteasome (prosome, macropain) subunit, alpha type, 5], PSMA6 [proteasome (prosome, macropain) subunit, alpha type, 6], PSMB8 [proteasome (prosome, macropain) subunit, beta type, 8 (large multifunctional peptidase 7)], PSMB9 [proteasome (prosome, macropain) subunit, beta type, 9 (large multifunctional peptidase 2)], PSMC1 [proteasome (prosome, macropain) 26S subunit, ATPase, 1], PSMC4 [proteasome (prosome, macropain) 26S subunit, ATPase, 4], PSMD9 [proteasome (prosome, macropain) 26S subunit, non-ATPase, 9], PSME1 [proteasome (prosome, macropain) activator subunit 1 (PA28 alpha)], PSME2 [proteasome (prosome, macropain) activator subunit 2 (PA28 beta)], PSMG1 [proteasome (prosome, macropain) assembly chaperone 1], PSPH [phosphoserine phosphatase], PSPN [persephin], PSTPIP1 [proline-serine-threonine phosphatase interacting protein 1], PTAFR [platelet-activating factor receptor], PTCH1 [patched homolog 1 (Drosophila)], PTCH2 [patched homolog 2 (Drosophila)], PTEN [phosphatase and tensin homolog], PTF1A [pancreas specific transcription factor, 1a], PTGER1 [prostaglandin E receptor 1 (subtype EP1), 42 kDa], PTGER2 [prostaglandin E receptor 2 (subtype EP2), 53 kDa], PTGER3 [prostaglandin E receptor 3 (subtype EP3)], PTGER4 [prostaglandin E receptor 4 (subtype EP4)], PTGES [prostaglandin E synthase], PTGES2 [prostaglandin E synthase 2], PTGIR [prostaglandin 12 (prostacyclin) receptor (IP)], PTGS 1 [prostaglandin-endoperoxide synthase 1 (prostaglandin G/H synthase and cyclooxygenase)], PTGS2 [prostaglandin-endoperoxide synthase 2 (prostaglandin G/H synthase and cyclooxygenase)], PTH [parathyroid hormone], PTH1R [parathyroid hormone 1 receptor], PTHLH [parathyroid hormone-like hormone], PTK2 [PTK2 protein tyrosine kinase 2], PTK2B [PTK2B protein tyrosine kinase 2 beta], PTK7 [PTK7 protein tyrosine kinase 7], PTN [pleiotrophin], PTPN1 [protein tyrosine phosphatase, non-receptor type 1], PTPN11 [protein tyrosine phosphatase, non-receptor type 11], PTPN13 [protein tyrosine phosphatase, non-receptor type 13 (AP0-1/CD95 (Fas)-associated phosphatase)], PTPN18 [protein tyrosine phosphatase, non-receptor type 18 (brain-derived)], PTPN2 [protein tyrosine phosphatase, non-receptor type 2], PTPN22 [protein tyrosine phosphatase, non-receptor type 22 (lymphoid)], PTPN6 [protein tyrosine phosphatase, non-receptor type 6], PTPN7 [protein tyrosine phosphatase, non-receptor type 7], PTPRA [protein tyrosine phosphatase, receptor type, A], PTPRB [protein tyrosine phosphatase, receptor type, B], PTPRC [protein tyrosine phosphatase, receptor type, C], PTPRD [protein tyrosine phosphatase, receptor type, D], PTPRE [protein tyrosine phosphatase, receptor type, E], PTPRF [protein tyrosine phosphatase, receptor type, F], PTPRJ [protein tyrosine phosphatase, receptor type, J], PTPRK [protein tyrosine phosphatase, receptor type, K], PTPRM [protein tyrosine phosphatase, receptor type, M], PTPRO [protein tyrosine phosphatase, receptor type, O], PTPRS [protein tyrosine phosphatase, receptor type, S], PTPRT [protein tyrosine phosphatase, receptor type, T], PTPRU [protein tyrosine phosphatase, receptor type, U], PTPRZ1 [protein tyrosine phosphatase, receptor-type, Z polypeptide 1], PTS [6-pyruvoyltetrahydropterin synthase], PTTG1 [pituitary tumor-transforming 1], PVR [poliovirus receptor], PVRL1 [poliovirus receptor-related 1 (herpesvirus entry mediator C)], PWP2 [PWP2 periodic tryptophan protein homolog (yeast)], PXN [paxillin], PYCARD [PYD and CARD domain containing], PYGB [phosphorylase, glycogen; brain], PYGM [phosphorylase, glycogen, muscle], PYY [peptide YY], QDPR [quinoid dihydropteridine reductase], QKI [quaking homolog, KH domain RNA binding (mouse)], RAB11A [RAB11A, member RAS oncogene family], RAB11FIP5 [RAB11 family interacting protein 5 (class I)], RAB39B [RAB39B, member RAS oncogene family], RAB3A [RAB3A, member RAS oncogene family], RAB4A [RAB4A, member RAS oncogene family], RAB5A [RABSA, member RAS oncogene family], RAB8A [RAB8A, member RAS oncogene family], RAB9A [RAB9A, member RAS oncogene family], RABEP1 [rabaptin, RAB GTPase binding effector protein 1], RABGEF1 [RAB guanine nucleotide exchange factor (GEF) 1], RAC1 [ras-related C3 botulinum toxin substrate 1 (rho family, small GTP binding protein Racl)], RAC2 [ras-related C3 botulinum toxin substrate 2 (rho family, small GTP binding protein Rac2)], RAC3 [ras-related C3 botulinum toxin substrate 3 (rho family, small GTP binding protein Rac3)], RAD51 [RAD51 homolog (RecA homolog, E. coli) (S. cerevisiae)], RAF1 [v-raf-1 murine leukemia viral oncogene homolog 1], RAG1 [recombination activating gene 1], RAG2 [recombination activating gene 2], RAGE [renal tumor antigen], RALA [v-ral simian leukemia viral oncogene homolog A (ras related)], RALBP1 [ralA binding protein 1], RALGAPA2 [Ral GTPase activating protein, alpha subunit 2 (catalytic)], RALGAPB [Ral GTPase activating protein, beta subunit (non-catalytic)], RALGDS [ral guanine nucleotide dissociation stimulator], RAN [RAN, member RAS oncogene family], RAP1A [RAP1A, member of RAS oncogene family], RAP1B [RAP1B, member of RAS oncogene family], RAP1GAP [RAP1 GTPase activating protein], RAPGEF3 [Rap guanine nucleotide exchange factor (GEF) 3], RAPGEF4 [Rap guanine nucleotide exchange factor (GEF) 4], RAPH1 [Ras association (RalGDS/AF-6) and pleckstrin homology domains 1], RAPSN [receptor-associated protein of the synapse], RARA [retinoic acid receptor, alpha], RARB [retinoic acid receptor, beta], RARG [retinoic acid receptor, gamma], RARS [arginyl-tRNA synthetase], RASA1 [RAS p21 protein activator (GTPase activating protein) 1], RASA2 [RAS p21 protein activator 2], RASGRF1 [Ras protein-specific guanine nucleotide-releasing factor 1], RASGRP1 [RAS guanyl releasing protein 1 (calcium and DAG-regulated)], RASSF1 [Ras association (RalGDS/AF-6) domain family member 1], RASSF5 [Ras association (RalGDS/AF-6) domain family member 5], RB1 [retinoblastoma 1], RBBP4 [retinoblastoma binding protein 4], RBM11 [RNA binding motif protein 11], RBM4 [RNA binding motif protein 4], RBM45 [RNA binding motif protein 45], RBP4 [retinol binding protein 4, plasma], RBPJ [recombination signal binding protein for immunoglobulin kappa J region], RCAN1 [regulator of calcineurin 1], RCAN2 [regulator of calcineurin 2], RCAN3 [ROAN family member 3], RCOR1 [REST corepressor 1], RDX [radixin], REEP3 [receptor accessory protein 3], REG1A [regenerating islet-derived 1 alpha], RELA [v-rel reticuloendotheliosis viral oncogene homolog A (avian)], RELN [reelin], REN [renin], REPIN1 [replication initiator 1], REST [RE1-silencing transcription factor], RET [ret proto-oncogene], RETN [resistin], RFC1 [replication factor C (activator 1) 1, 145 kDa], RFC2 [replication factor C (activator 1) 2, 40 kDa], RFX1 [regulatory factor X, 1 (influences HLA class II expression)], RGMA [RGM domain family, member A], RGMB [RGM domain family, member B], RGS3 [regulator of G-protein signaling 3], RHD [Rh blood group, D antigen], RHEB [Ras homolog enriched in brain], RHO [rhodopsin], RHOA [ras homolog gene family, member A], RHOB [ras homolog gene family, member B], RHOC [ras homolog gene family, member C], RHOD [ras homolog gene family, member D], RHOG [ras homolog gene family, member G (rho G)], RHOH [ras homolog gene family, member H], RICTOR [RPTOR independent companion of MTOR, complex 2], RIMS3 [regulating synaptic membrane exocytosis 3], RIPK1 [receptor (TNFRSF)-interacting serine-threonine kinase 1], RIPK2 [receptor-interacting serine-threonine kinase 2], RNASE1 [ribonuclease, RNase A family, 1 (pancreatic)], RNASE3 [ribonuclease, RNase A family, 3 (eosinophil cationic protein)], RNASEL [ribonuclease L (2′ 5′-oligoisoadenylate synthetase-dependent)], RND1 [Rho family GTPase 1], RND2 [Rho family GTPase 2], RND3 [Rho family GTPase 3], RNF123 [ring finger protein 123], RNF128 [ring finger protein 128], RNF13 [ring finger protein 13], RNF135 [ring finger protein 135], RNF2 [ring finger protein 2], RNF6 [ring finger protein (C3H2C3 type) 6], RNH1 [ribonuclease/angiogenin inhibitor 1], RNPC3 [RNA-binding region (RNP1, RRM) containing 3], ROBO1 [roundabout, axon guidance receptor, homolog 1 (Drosophila)], ROBO2 [roundabout, axon guidance receptor, homolog 2 (Drosophila)], ROBO3 [roundabout, axon guidance receptor, homolog 3 (Drosophila)], ROBO4 [roundabout homolog 4, magic roundabout (Drosophila)], ROCK1 [Rho-associated, coiled-coil containing protein kinase 1], ROCK2 [Rho-associated, coiled-coil containing protein kinase 2], RPGR [retinitis pigmentosa GTPase regulator], RPGRIP1 [retinitis pigmentosa GTPase regulator interacting protein 1], RPGRIP1L [RPGRIPI-like], RPL10 [ribosomal protein L10], RPL24 [ribosomal protein L24], RPL5 [ribosomal protein L5], RPL7A [ribosomal protein L7a], RPLP0 [ribosomal protein, large, P0], RPS17 [ribosomal protein S17], RPS17P3 [ribosomal protein S17 pseudogene 3], RPS19 [ribosomal protein S19], RPS27A [ribosomal protein S27a], RPS6 [ribosomal protein S6], RPS6KA1 [ribosomal protein S6 kinase, 90 kDa, polypeptide 1], RPS6KA3 [ribosomal protein S6 kinase, 90 kDa, polypeptide 3], RPS6KA6 [ribosomal protein S6 kinase, 90 kDa, polypeptide 6], RPS6KB1 [ribosomal protein S6 kinase, 70 kDa, polypeptide 1], RRAS [related RAS viral (r-ras) oncogene homolog], RRAS2 [related RAS viral (r-ras) oncogene homolog 2], RRBP1 [ribosome binding protein 1 homolog 180 kDa (dog)], RRM1 [ribonucleotide reductase M1], RRM2 [ribonucleotide reductase M2], RRM2B [ribonucleotide reductase M2 B (TP53 inducible)], RTN4 [reticulon 4], RTN4R [reticulon 4 receptor], RUFY3 [RUN and FYVE domain containing 3], RUNX1 [runt-related transcription factor 1], RUNX1T1 [runt-related transcription factor 1; translocated to, 1 (cyclin D-related)], RUNX2 [runt-related transcription factor 2], RUNX3 [runt-related transcription factor 3], RUVBL2 [RuvB-like 2 (E. coli)], RXRA [retinoid X receptor, alpha], RYK [RYK receptor-like tyrosine kinase], RYR2 [ryanodine receptor 2 (cardiac)], RYR3 [ryanodine receptor 3], S100A1 [S 100 calcium binding protein A1], S100A10 [S 100 calcium binding protein A10], S100A12 [S 100 calcium binding protein A12], S100A2 [S100 calcium binding protein A2], S100A4 [S100 calcium binding protein A4], S100A6 [S100 calcium binding protein A6], S100A7 [S100 calcium binding protein A7], S100A8 [S 100 calcium binding protein A8], S100A9 [S 100 calcium binding protein A9], S100B [S 100 calcium binding protein B], SAA4 [serum amyloid A4, constitutive], SACS [spastic ataxia of Charlevoix-Saguenay (sacsin)], SAFB [scaffold attachment factor B], SAG [S-antigen; retina and pineal gland (arrestin)], SAMHD1 [SAM domain and HD domain 1], SATB2 [SATB homeobox 2], SBDS [Shwachman-Bodian-Diamond syndrome], SCARB1 [scavenger receptor class B, member 1], SCD [stearoyi-CoA desaturase (delta-9-desaturase)], SCD5 [stearoyl-CoA desaturase 5], SCG2 [secretogranin II], SCG5 [secretogranin V (7B2 protein)], SCGB1A1 [secretoglobin, family 1A, member 1 (uteroglobin)], SCN11A [sodium channel, voltage-gated, type XI, alpha subunit], SCN1A [sodium channel, voltage-gated, type I, alpha subunit], SCN2A [sodium channel, voltage-gated, type II, alpha subunit], SCN3A [sodium channel, voltage-gated, type III, alpha subunit], SCN5A [sodium channel, voltage-gated, type V, alpha subunit], SCN7A [sodium channel, voltage-gated, type VII, alpha], SCNN1B [sodium channel, nonvoltage-gated 1, beta], SCNN1G [sodium channel, nonvoltage-gated 1, gamma], SCP2 [sterol carrier protein 2], SCT [secretin], SCTR [secretin receptor], SCUBE1 [signal peptide, CUB domain, EGF-like 1], SDC2 [syndecan 2], SDC3 [syndecan 3], SDCBP [syndecan binding protein (syntenin)], SDHB [succinate dehydrogenase complex, subunit B, iron sulfur (Ip)], SDHD [succinate dehydrogenase complex, subunit D, integral membrane protein], SDS [serine dehydratase], SEC14L2 [SEC 14-like 2 (S. cerevisiae)], SELE [selectin E], SELL [selectin L], SELP [selectin P (granule membrane protein 140 kDa, antigen CD62)], SELPLG [selectin P ligand], SEMA3A [sema domain, immunoglobulin domain (Ig), short basic domain, secreted, (semaphorin) 3A], SEMA3B [sema domain, immunoglobulin domain (Ig), short basic domain, secreted, (semaphorin) 3B], SEMA3C [sema domain, immunoglobulin domain (Ig), short basic domain, secreted, (semaphorin) 30], SEMA3D [sema domain, immunoglobulin domain (Ig), short basic domain, secreted, (semaphorin) 3D], SEMA3E [sema domain, immunoglobulin domain (Ig), short basic domain, secreted, (semaphorin) 3E], SEMA3F [sema domain, immunoglobulin domain (Ig), short basic domain, secreted, (semaphorin) 3F], SEMA3G [sema domain, immunoglobulin domain (Ig), short basic domain, secreted, (semaphorin) 3G], SEMA4A [sema domain, immunoglobulin domain (Ig), transmembrane domain (TM) and short cytoplasmic domain, (semaphorin) 4A], SEMA4B [sema domain, immunoglobulin domain (Ig), transmembrane domain (TM) and short cytoplasmic domain, (semaphorin) 4B], SEMA4C [sema domain, immunoglobulin domain (Ig), transmembrane domain (TM) and short cytoplasmic domain, (semaphorin) 40], SEMA4D [sema domain, immunoglobulin domain (Ig), transmembrane domain (TM) and short cytoplasmic domain, (semaphorin) 4D], SEMA4F [sema domain, immunoglobulin domain (Ig), transmembrane domain (TM) and short cytoplasmic domain, (semaphorin) 4F], SEMA4G [sema domain, immunoglobulin domain (Ig), transmembrane domain (TM) and short cytoplasmic domain, (semaphorin) 4G], SEMA5A [sema domain, seven thrombospondin repeats (type 1 and type 1-like), transmembrane domain (TM) and shmi cytoplasmic domain, (semaphorin) SA], SEMA5B [sema domain, seven thrombospondin repeats (type 1 and type 1-like), transmembrane domain (TM) and short cytoplasmic domain, (semaphorin) 5B], SEMA6A [sema domain, transmembrane domain (TM), and cytoplasmic domain, (semaphorin) 6A], SEMA6B [sema domain, transmembrane domain (TM), and cytoplasmic domain, (semaphorin) 6B], SEMA6C [sema domain, transmembrane domain (TM), and cytoplasmic domain, (semaphorin) 60], SEMA6D [sema domain, transmembrane domain (TM), and cytoplasmic domain, (semaphorin) 6D], SEMA7A [semaphorin 7A, GP1 membrane anchor (John Milton Hagen blood group)], SEPP1 [selenoprotein P, plasma, 1], SEPT2 [septin 2], SEPT4 [septin 4], SEPT5 [septin 5], SEPT6 [septin 6], SEPT7 [septin 7], SEPT9 [septin 9], SERPTNA1 [serpin peptidase inhibitor, clade A (alpha-1 antiproteinase, antitrypsin), member 1], SERPINA3 [serpin peptidase inhibitor, clade A (alpha-1 antiproteinase, antitrypsin), member 3], SERPINA7 [serpin peptidase inhibitor, clade A (alpha-1 antiproteinase, antitrypsin), member 7], SERPINB1 [serpin peptidase inhibitor, clade B (ovalbumin), member 1], SERPINB2 [serpin peptidase inhibitor, clade B (ovalbumin), member 2], SERPINB6 [serpin peptidase inhibitor, clade B (ovalbumin), member 6], SERPTNC1 [serpin peptidase inhibitor, clade C (antithrombin), member 1], SERPINE1 [serpin peptidase inhibitor, clade E (nexin, plasminogen activator inhibitor type 1), member 1], SERPINE2 [serpin peptidase inhibitor, clade E (nexin, plasminogen activator inhibitor type 1), member 2], SERPINF1 [serpin peptidase inhibitor, clade F (alpha-2 antiplasmin, pigment epithelium derived factor), member 1], SERPINH1 [serpin peptidase inhibitor, clade H (heat shock protein 47), member 1, (collagen binding protein 1)1, SERPINI1 [serpin peptidase inhibitor, clade I (neuroserpin), member 1], SET [SET nuclear oncogene], SETX [senataxin], SEZ6L2 [seizure related 6 homolog (mouse)-like 2], SFPQ [splicing factor proline/glutamine-rich (polypyrimidine tract binding protein associated)], SFRP1 [secreted frizzled-related protein 1], SFRP4 [secreted frizzled-related protein 4], SFRS15 [splicing factor, arginine/serine-rich 15], SFTPA1 [surfactant protein A1], SFTPB [surfactant protein B], SFTPC [surfactant protein C], SGCB [sarcoglycan, beta (43 kDa dystrophin-associated glycoprotein)], SGCE [sarcoglycan, epsilon], SGK1 [serum/glucocorticoid regulated kinase 1], SH2B1 [SH2B adaptor protein 1], SH2B3 [SH2B adaptor protein 3], SH2D1A [SH2 domain containing 1A], SH3BGR [SH3 domain binding glutamic acid-rich protein], SH3BGRL [SH3 domain binding glutamic acid-rich protein like], SH3BP1 [SH3-domain binding protein 1], SH3GL1P2 [SH3-domain GRB2-like 1 pseudogene 2], SH3GL3 [SH3-domain GRB2-like 3], SH3KBP1 [SH3-domain kinase binding protein 1], SH3PXD2A [SH3 and PX domains 2A], SHANK1 [SH3 and multiple ankyrin repeat domains 1], SHANK2 [SH3 and multiple ankyrin repeat domains 2], SHANK3 [SH3 and multiple ankyrin repeat domains 3], SHBG [sex hormone-binding globulin], SHC1 [SHC (Src homology 2 domain containing) transforming protein 1], SHC3 [SHC (Src homology 2 domain containing) transforming protein 3], SHH [sonic hedgehog homolog (Drosophila)], SHOC2 [soc-2 suppressor of clear homolog (C. elegans)], SI [sucrase-isomaltase (alpha-glucosidase)], SIAH1 [seven in absentia homolog 1 (Drosophila)], SIAH2 [seven in absentia homolog 2 (Drosophila)], SIGMAR1 [sigma non-opioid intracellular receptor 1], SILV [silver homolog (mouse)], SIM1 [single-minded homolog 1 (Drosophila)], SIM2 [single-minded homolog 2 (Drosophila)], SIP1 [survival of motor neuron protein interacting protein 1], SIRPA [signal-regulatory protein alpha], SIRT1 [sirtuin (silent mating type information regulation 2 homolog) 1 (S. cerevisiae)], SIRT4 [sirtuin (silent mating type information regulation 2 homolog) 4 (S. cerevisiae)], SIRT6 [sirtuin (silent mating type information regulation 2 homolog) 6 (S. cerevisiae)], SIX5 [SIX homeobox 5], SKI [v-ski sarcoma viral oncogene homolog (avian)], SKP2 [S-phase kinase-associated protein 2 (p45)], SLAMF6 [SLAM family member 6], SLC10A1 [solute carrier family 10 (sodium/bile acid cotransporter family), member 1], SLC11A2 [solute carrier family 11 (proton-coupled divalent metal ion transporters), member 2], SLC12A1 [solute carrier family 12 (sodium/potassium/chloride transporters), member 1], SLC12A2 [solute carrier family 12 (sodium/potassium/chloride transporters), member 2], SLC12A3 [solute carrier family 12 (sodium/chloride transporters), member 3], SLC12A5 [solute carrier family 12 (potassium/chloride transporter), member 5], SLC12A6 [solute carrier family 12 (potassium/chloride transporters), member 6], SLC13A1 [solute carrier family 13 (sodium/sulfate symporters), member 1], SLC15A1 [solute carrier family 15 (oligopeptide transporter), member 1], SLC16A2 [solute carrier family 16, member 2 (monocarboxylic acid transporter 8)], SLC17A5 [solute carrier family 17 (anion/sugar transporter), member 5], SLC17A7 [solute carrier family 17 (sodium-dependent inorganic phosphate cotransporter), member 7], SLC18A2 [solute carrier family 18 (vesicular monoamine), member 2], SLC18A3 [solute carrier family 18 (vesicular acetylcholine), member 3], SLC19A1 [solute carrier family 19 (folate transporter), member 1], SLC19A2 [solute carrier family 19 (thiamine transporter), member 2], SLC1A1 [solute carrier family 1 (neuronal/epithelial high affinity glutamate transporter, system Xag), member 1], SLC1A2 [solute carrier family 1 (glial high affinity glutamate transporter), member 2], SLC1A3 [solute carrier family 1 (glial high affinity glutamate transporter), member 3], SLC22A2 [solute carrier family 22 (organic cation transporter), member 2], SLC25A12 [solute carrier family 25 (mitochondrial carrier, Aralar), member 12], SLC25A13 [solute carrier family 25, member 13 (citrin)], SLC25A20 [solute carrier family 25 (carnitine/acylcarnitine translocase), member 20], SLC25A3 [solute carrier family 25 (mitochondrial carrier; phosphate carrier), member 3], SLC26A3 [solute carrier family 26, member 3], SLC27A1 [solute carrier family 27 (fatty acid transporter), member 1], SLC29A1 [solute carrier family 29 (nucleoside transporters), member 1], SLC2A1 [solute carrier family 2 (facilitated glucose transporter), member 1], SLC2A13 [solute carrier family 2 (facilitated glucose transporter), member 13], SLC2A2 [solute carrier family 2 (facilitated glucose transporter), member 2], SLC2A3 [solute carrier family 2 (facilitated glucose transporter), member 3], SLC2A4 [solute carrier family 2 (facilitated glucose transporter), member 4], SLC30A3 [solute carrier family 30 (zinc transporter), member 3], SLC30A4 [solute carrier family 30 (zinc transporter), member 4], SLC30A8 [solute carrier family 30 (zinc transporter), member 8], SLC31A1 [solute carrier family 31 (copper transporters), member 1], SLC32A1 [solute carrier family 32 (GABA vesicular transporter), member 1], SLC34A1 [solute carrier family 34 (sodium phosphate), member 1], SLC38A3 [solute carrier family 38, member 3], SLC39A2 [solute carrier family 39 (zinc transporter), member 2], SLC39A3 [solute carrier family 39 (zinc transporter), member 3], SLC40A1 [solute carrier family 40 (iron-regulated transporter), member 1], SLC4A11 [solute carrier family 4, sodium borate transpmier, member 11], SLC5A3 [solute carrier family 5 (sodium/myo-inositol cotransporter), member 3], SLC5A8 [solute carrier family 5 (iodide transporter), member 8], SLC6A1 [solute carrier family 6 (neurotransmitter transporter, GABA), member 1], SLC6A14 [solute carrier family 6 (amino acid transporter), member 14], SLC6A2 [solute carrier family 6 (neurotransmitter transporter, noradrenalin), member 2], SLC6A3 [solute carrier family 6 (neurotransmitter transporter, dopamine), member 3], SLC6A4 [solute carrier family 6 (neurotransmitter transporter, serotonin), member 4], SLC6A8 [solute carrier family 6 (neurotransmitter transporter, creatine), member 8], SLC7A14 [solute carrier family 7 (cationic amino acid transporter, y+ system), member 14], SLC7A5 [solute carrier family 7 (cationic amino acid transporter, y+ system), member 5], SLC9A2 [solute carrier family 9 (sodium/hydrogen exchanger), member 2], SLC9A3 [solute carrier family 9 (sodium/hydrogen exchanger), member 3], SLC9A3R1 [solute carrier family 9 (sodium/hydrogen exchanger), member 3 regulator 1], SLC9A3R2 [solute carrier family 9 (sodium/hydrogen exchanger), member 3 regulator 2], SLC9A6 [solute carrier family 9 (sodium/hydrogen exchanger), member 6], SLIT1 [slit homolog 1 (Drosophila)], SLIT2 [slit homolog 2 (Drosophila)], SLIT3 [slit homolog 3 (Drosophila)], SLITRK1 [SLIT and NTRK-Iike family, member 1], SLN [sarcolipin], SLPI [secretory leukocyte peptidase inhibitor], SMAD1 [SMAD family member 1], SMAD2 [SMAD family member 2], SMAD3 [SMAD family member 3], SMAD4 [SMAD family member 4], SMAD6 [SMAD family member 6], SMAD7 [SMAD family member 7], SMARCA1 [SWI/SNF related, matrix associated, actin dependent regulator of chromatin, subfamily a, member 1], SMARCA2 [SWI/SNF related, matrix associated, actin dependent regulator of chromatin, subfamily a, member 2], SMARCA4 [SWI/SNF related, matrix associated, actin dependent regulator of chromatin, subfamily a, member 4], SMARCA5 [SWI/SNF related, matrix associated, actin dependent regulator of chromatin, subfamily a, member 5], SMARCB1 [SWI/SNF related, matrix associated, actin dependent regulator of chromatin, subfamily b, member 1], SMARCC1 [SWI/SNF related, matrix associated, actin dependent regulator of chromatin, subfamily c, member 1], SMARCC2 [SWI/SNF related, matrix associated, actin dependent regulator of chromatin, subfamily c, member 2], SMARCD1 [SWI/SNF related, matrix associated, actin dependent regulator of chromatin, subfamily d, member 1], SMARCD3 [SWI/SNF related, matrix associated, actin dependent regulator of chromatin, subfamily d, member 3], SMARCE1 [SWI/SNF related, matrix associated, actin dependent regulator of chromatin, subfamily e, member 1], SMG1 [SMG1 homolog, phosphatidylinositol 3-kinase-related kinase (C. elegans)], SMN1 [survival of motor neuron 1, telomeric], SMO [smoothened homolog (Drosophila)], SMPD1 [sphingomyelin phosphodiesterase 1, acid lysosomal], SMS [spermine synthase], SNAI2 [snail homolog 2 (Drosophila)], SNAP25 [synaptosomal-associated protein, 25 kDa], SNCA [synuclein, alpha (non A4 component of amyloid precursor)], SNCAIP [synuclein, alpha interacting protein], SNOB [synuclein, beta], SNCG [synuclein, gamma (breast cancer-specific protein 1)], SNRPA [small nuclear ribonucleoprotein polypeptide A], SNRPN [small nuclear ribonucleoprotein polypeptide N], SNTG2 [syntrophin, gamma 2], SNURF [SNRPN upstream reading frame], SOAT1 [sterol O-acyltransferase 1], SOCS1 [suppressor of cytokine signaling 1], SOCS3 [suppressor of cytokine signaling 3], SOD1 [superoxide dismutase 1, soluble], SOD2 [superoxide dismutase 2, mitochondrial], SORBS3 [sorbin and SH3 domain containing 3], SORL1 [sortilin-related receptor, L(DLR class) A repeats-containing], SORT1 [sortilin 1], SOS1 [son of sevenless homolog 1 (Drosophila)], SOS2 [son of sevenless homolog 2 (Drosophila)], SOSTDC1 [sclerostin domain containing 1], SOX1 [SRY (sex determining region Y)-box 1], SOX10 [SRY (sex determining region Y)-box 10], SOX18 [SRY (sex determining region Y)-box 18], SOX2 [SRY (sex determining region Y)-box 2], SOX3 [SRY (sex determining region Y)-box 3], SOX9 [SRY (sex determining region Y)-box 9], SP1 [Sp1 transcription factor], SP3 [Sp3 transcription factor], SPANXB1 [SPANX family, member B1], SPANXC [SPANX family, member C], SPARC [secreted protein, acidic, cysteine-rich (osteonectin)], SPARCL1 [SPARC-like 1 (hevin)], SPAST [spastin], SPHK1 [sphingosine kinase 1], SPINK1 [serine peptidase inhibitor, Kazal type 1], SPINT2 [serine peptidase inhibitor, Kunitz type, 2], SPN [sialophorin], SPNS2 [spinster homolog 2 (Drosophila)], SPON2 [spondin 2, extracellular matrix protein], SPP1 [secreted phosphoprotein 1], SPRED2 [sprouty-related, EVH1 domain containing 2], SPRY2 [sprouty homolog 2 (Drosophila)], SPTA1 [spectrin, alpha, erythrocytic 1 (elliptocytosis 2)], SPTAN1 [spectrin, alpha, non-erythrocytic 1 (alpha-fodrin)], SPTB [spectrin, beta, erythrocytic], SPTBN1 [spectrin, beta, non-erythrocytic 1], SRC [v-src sarcoma (Schmidt-Ruppin A-2) viral oncogene homolog (avian)], SRCRB4D [scavenger receptor cysteine rich domain containing, group B (4 domains)], SRD5A1 [steroid-5-alpha-reductase, alpha polypeptide 1 (3-oxo-5 alpha-steroid delta 4-dehydrogenase alpha 1)], SREBF1 [sterol regulatory element binding transcription factor 1], SREBF2 [sterol regulatory element binding transcription factor 2], SRF [serum response factor (c-fos serum response element-binding transcription factor)], SRGAP1 [SLIT-ROBO Rho GTPase activating protein 1], SRGAP2 [SLIT-ROBO Rho GTPase activating protein 2], SRGAP3 [SLIT-ROBO Rho GTPase activating protein 3], SRPX [sushi-repeat-containing protein, X-linked], SRY [sex determining region Y], SSB [Sjogren syndrome antigen B (autoantigen La)], SSH1 [slingshot homolog 1 (Drosophila)], SSRP1 [structure specific recognition protein 1], SST [somatostatin], SSTR1 [somatostatin receptor 1], SSTR2 [somatostatin receptor 2], SSTR3 [somatostatin receptor 3], SSTR4 [somatostatin receptor 4], SSTR5 [somatostatin receptor 5], ST13 [suppression of tumorigenicity 13 (colon carcinoma) (Hsp70 interacting protein)], ST14 [suppression of tumorigenicity 14 (colon carcinoma)], ST6GAL1 [ST6 beta-galactosamide alpha-2 [6-sialyltranferase 1], ST7 [suppression of tumorigenicity 7], STAG2 [stromal antigen 2], STAG3 [stromal antigen 3], STAR [steroidogenic acute regulatory protein], STAT1 [signal transducer and activator of transcription 1, 91 kDa], STAT2 [signal transducer and activator of transcription 2, 113 kDa], STAT3 [signal transducer and activator of transcription 3 (acute-phase response factor)], STAT4 [signal transducer and activator of transcription 4], STAT5A [signal transducer and activator of transcription 5A], STAT5B [signal transducer and activator of transcription 5B], STAT6 [signal transducer and activator of transcription 6, interleukin-4 induced], STATH [statherin], STC1 [stanniocalcin 1], STIL [SCL/TAL1 interrupting locus], STIM1 [stromal interaction molecule 1], STK11 [serine/threonine kinase 11], STK24 [serine/threonine kinase 24 (STE20 homolog, yeast)], STK36 [serine/threonine kinase 36, fused homolog (Drosophila)], STK38 [serine/threonine kinase 38], STK38L [serine/threonine kinase 38 like], STK39 [serine threonine kinase 39 (STE20/SPS1 homolog, yeast)], STMN1 [stathmin 1], STMN2 [stathmin-like 2], STMN3 [stathmin-like 3], STMN4 [stathmin-like 4], STOML1 [stomatin (EPB72)-like 1], STS [steroid sulfatase (microsomal), isozyme S], STUB1 [STIP1 homology and U-box containing protein 1], STX1A [syntaxin 1A (brain)], STX3 [syntaxin 3], STYX [serine/threonine/tyrosine interacting protein], SUFU [suppressor of fused homolog (Drosophila)], SULT2A1 [sulfotransferase family, cytosolic, 2A, dehydroepiandrosterone (DHEA)-preferring, member 1], SUMO1 [SMT3 suppressor of mif two 3 homolog 1 (S. cerevisiae)], SUMO3 [SMT3 suppressor of mif two 3 homolog 3 (S. cerevisiae)], SUN1 [Sad1 and UNC84 domain containing 1], SUN2 [Sadl and UNC84 domain containing 2], SUPT16H [suppressor of Ty 16 homolog (S. cerevisiae)], SUZ12P [suppressor of zeste 12 homolog pseudogene], SV2A [synaptic vesicle glycoprotein 2A], SYK [spleen tyrosine kinase], SYN1 [synapsin I], SYN2 [synapsin II], SYN3 [synapsin III], SYNGAP1 [synaptic Ras GTPase activating protein 1 homolog (rat)], SYNJ1 [synaptojanin 1], SYNPO2 [synaptopodin 2], SYP [synaptophysin], SYT1 [synaptotagmin I], TAC1 [tachykinin, precursor 1], TAC3 [tachykinin 3], TACR1 [tachykinin receptor 1], TAF1 [TAF1 RNA polymerase II, TATA box binding protein (TBP)-associated factor, 250 kDa], TAF6 [TAF6 RNA polymerase II, TATA box binding protein (TBP)-associated factor, 80 kDa], TAGAP [T-cell activation RhoGTPase activating protein], TAGLN [transgelin], TAGLN3 [transgelin 3], TAOK2 [TAO kinase 2], TAP1 [transporter 1, ATP-binding cassette, sub-family B (MDR/TAP)], TAP2 [transporter 2, ATP-binding cassette, sub-family B (MDR/TAP)], TAPBP [TAP binding protein (tapasin)], TARDBP [TAR DNA binding protein], TARP [TCR gamma alternate reading frame protein], TAS2R1 [taste receptor, type 2, member 1], TAT [tyrosine aminotransferase], TBC1D4 [TBC1 domain family, member 4], TBCB [tubulin folding cofactor B], TBCD [tubulin folding cofactor D], TBCE [tubulin folding cofactor E], TBL1Y [transducin (beta)-like 1, Y-linked], TBL2 [transducin (beta)-like 2], TBP [TATA box binding protein], TBPL2 [TATA box binding protein like 2], TBR1 [T-box, brain, 1], TBX1 [T-box 1], TBX21 [T-box 21], TBXA2R [thromboxane A2 receptor], TBXAS1 [thromboxane A synthase 1 (platelet)], TCEB3 [transcription elongation factor B (SIII), polypeptide 3 (110 kDa, elongin A)], TCF12 [transcription factor 12], TCF19 [transcription factor 19], TCF4 [transcription factor 4], TCF7 [transcription factor 7 (T-cell specific, HMG-box)], TCF7L2 [transcription factor 7-like 2 (T-cell specific, HMG-box)], TCHH [trichohyalin], TCN1 [transcobalamin I (vitamin B12 binding protein, R binder family)], TCN2 [transcobalamin II; macrocytic anemia], TCP1 [t-complex 1], TD02 [tryptophan 2 [3-dioxygenase], TDRD3 [tudor domain containing 3], TEAD2 [TEA domain family member 2], TEAD4 [TEA domain family member 4], TEK [TEK tyrosine kinase, endothelial], TERF1 [telomeric repeat binding factor (NIMA-interacting) 1], TERF2 [telomeric repeat binding factor 2], TERT [telomerase reverse transcriptase], TET2 [tet oncogene family member 2], TF [transferrin], TFAM [transcription factor A, mitochondrial], TFAP2A [transcription factor AP-2 alpha (activating enhancer binding protein 2 alpha)], TFCP2 [transcription factor CP2], TFF1 [trefoil factor 1], TFF2 [trefoil factor 2], TFF3 [trefoil factor 3 (intestinal)], TFPI [tissue factor pathway inhibitor (lipoprotein-associated coagulation inhibitor)], TFPI2 [tissue factor pathway inhibitor 2], TFRC [transferrin receptor (p90, CD71)], TG [thyroglobulin], TGFa [transforming growth factor, alpha], TGFB1 [transforming growth factor, beta 1], TGFB I1 [transforming growth factor beta 1 induced transcript 1], TGFB2 [transforming growth factor, beta 2], TGFB3 [transforming growth factor, beta 3], TGFBR1 [transforming growth factor, beta receptor 1], TGFBR2 [transforming growth factor, beta receptor II (70/80 kDa)], TGFBR3 [transforming growth factor, beta receptor III], TGIF1 [TGFB-induced factor homeobox 1], TGM2 [transglutaminase 2 (C polypeptide, protein-glutamine-gamma-glutamyltransferase)], TH [tyrosine hydroxylase], THAP1 [THAP domain containing, apoptosis associated protein 1], THBD [thrombomodulin], THBS1 [thrombospondin 1], THBS2 [thrombospondin 2], THBS4 [thrombospondin 4], THEM4 [thioesterase superfamily member 4], THPO [thrombopoietin], THRA [thyroid hormone receptor, alpha (erythroblastic leukemia viral (v-erb-a) oncogene homolog, avian)], THY1 [Thy-1 cell surface antigen], TIAM1 [T-celllymphoma invasion and metastasis 1], TIAM2 [T-cell lymphoma invasion and metastasis 2], TIMP1 [TIMP metallopeptidase inhibitor 1], TIMP2 [TIMP metallopeptidase inhibitor 2], TIMP3 [TIMP metallopeptidase inhibitor 3], TINF2 [TERF1 (TRF1)-interacting nuclear factor 2], TJP1 [tight junction protein 1 (zona occludens 1)], TJP2 [tight junction protein 2 (zona occludens 2)], TK1 [thymidine kinase 1, soluble], TKT [transketolase], TLE1 [transducin-like enhancer of split 1 (E(sp1) homolog, Drosophila)], TLR1 [toll-like receptor 1], TLR2 [toll-like receptor 2], TLR3 [toll-like receptor 3], TLR4 [toll-like receptor 4], TLR5 [toll-like receptor 5], TLR7 [toll-like receptor 7], TLR8 [toll-like receptor 8], TLR9 [toll-like receptor 9], TLX3 [T-cell leukemia homeobox 3], TMEFF1 [transmembrane protein with EGF-like and two follistatin-like domains 1], TMEM100 [transmembrane protein 100], TMEM216 [transmembrane protein 216], TMEM50B [transmembrane protein 50B], TMEM67 [transmembrane protein 67], TMEM70 [transmembrane protein 70], TMEM87A [transmembrane protein 87A], TMOD2 [tropomodulin 2 (neuronal)], TMOD4 [tropomodulin 4 (muscle)], TMPRSS11A [transmembrane protease, serine 11A], TMPRSS15 [transmembrane protease, serine 15], TMPRSS2 [transmembrane protease, serine 2], TNC [tenascin C], TNF [tumor necrosis factor (TNF superfamily, member 2)], TNFAIP3 [tumor necrosis factor, alpha-induced protein 3], TNFRSF10A [tumor necrosis factor receptor superfamily, member 10a], TNFRSF10B [tumor necrosis factor receptor superfamily, member 10b], TNFRSF10C [tumor necrosis factor receptor superfamily, member 10c, decoy without an intracellular domain], TNFRSF10D [tumor necrosis factor receptor superfamily, member 10d, decoy with truncated death domain], TNFRSF11B [tumor necrosis factor receptor superfamily, member 11b], TNFRSF18 [tumor necrosis factor receptor superfamily, member 18], TNFRSF19 [tumor necrosis factor receptor superfamily, member 19], TNFRSF1A [tumor necrosis factor receptor superfamily, member 1A], TNFRSF1B [tumor necrosis factor receptor superfamily, member IB], TNFRSF25 [tumor necrosis factor receptor superfamily, member 25], TNFRSF8 [tumor necrosis factor receptor superfamily, member 8], TNFSF10 [tumor necrosis factor (ligand) superfamily, member 10], TNFSF11 [tumor necrosis factor (ligand) superfamily, member 11], TNFSF13 [tumor necrosis factor (ligand) superfamily, member 13], TNFSF13B [tumor necrosis factor (ligand) superfamily, member 13b], TNFSF4 [tumor necrosis factor (ligand) superfamily, member 4], TNK2 [tyrosine kinase, non-receptor, 2], TNN13 [troponin I type 3 (cardiac)], TNNT1 [troponin T type 1 (skeletal, slow)], TNNT2 [troponin T type 2 (cardiac)], TNR [tenascin R (restrictin, janusin)], TNS1 [tensin 1], TNS3 [tensin 3], TNXB [tenascin XB], TOLLIP [toll interacting protein], TOP1 [topoisomerase (DNA) I], TOP2A [topoisomerase (DNA) II alpha 170 kDa], TOP2B [topoisomerase (DNA) II beta 180 kDa], TOR1A [torsin family 1, member A (torsin A)], TP53 [tumor protein p53], TP53BP1 [tumor protein p53 binding protein 1], TP63 [tumor protein p63], TP73 [tumor protein p73], TPH1 [tryptophan hydroxylase 1], TPH2 [tryptophan hydroxylase 2], TPI1 [triosephosphate isomerase 1], TPO [thyroid peroxidase], TPT1 [tumor protein, translationally-controlled 1], TPTE [transmembrane phosphatase with tensin homology], TRADD [TNFRSF1A-associated via death domain], TRAF2 [TNF receptor-associated factor 2], TRAF3 [TNF receptor-associated factor 3], TRAF6 [TNF receptor-associated factor 6], TRAP1 [TNF receptor-associated protein 1], TREM1 [triggering receptor expressed on myeloid cells 1], TRH [thyrotropin-releasing hormone], TRIM21 [tripartite motif-containing 21], TRIM22 [tripartite motif-containing 22], TRIM26 [tripartite motif-containing 26], TRIM27 [tripartite motif-containing 27], TRIM50 [tripartite motif-containing 50], TRIO [triple functional domain (PTPRF interacting)], TRPA1 [transient receptor potential cation channel, subfamily A, member 1], TRPC1 [transient receptor potential cation channel, subfamily C, member 1], TRPC5 [transient receptor potential cation channel, subfamily C, member 5], TRPC6 [transient receptor potential cation channel, subfamily C, member 6], TRPM1 [transient receptor potential cation channel, subfamily M, member 1], TRPV1 [transient receptor potential cation channel, subfamily V, member 1], TRPV2 [transient receptor potential cation channel, subfamily V, member 2], TRRAP [transformation/transcription domain-associated protein], TSC1 [tuberous sclerosis 1], TSC2 [tuberous sclerosis 2], TSC22D3 [TSC22 domain family, member 3], TSG101 [tumor susceptibility gene 101], TSHR [thyroid stimulating hormone receptor], TSN [translin], TSPAN12 [tetraspanin 12], TSPAN7 [tetraspanin 7], TSPO [translocator protein (18 kDa)], TTC3 [tetratricopeptide repeat domain 3], TTF1 [transcription termination factor, RNA polymerase I], TTF2 [transcription termination factor, RNA polymerase II], TTN [titin], TTPA [tocopherol (alpha) transfer protein], TTR [transthyretin], TUB [tubby homolog (mouse)], TUBA1A [tubulin, alpha 1a], TUBA1B [tubulin, alpha 1b], TUBAIC [tubulin, alpha 1c], TUBA3C [tubulin, alpha 3c], TUBA3D [tubulin, alpha 3d], TUBA4A [tubulin, alpha 4a], TUBA8 [tubulin, alpha 8], TUBB [tubulin, beta], TUBB1 [tubulin, beta 1], TUBB2A [tubulin, beta 2A], TUBB2B [tubulin, beta 2B], TUBB2C [tubulin, beta 20], TUBB3 [tubulin, beta 3], TUBB4 [tubulin, beta 4], TUBB4Q [tubulin, beta polypeptide 4, member Q], TUBB6 [tubulin, beta 6], TUBGCP5 [tubulin, gamma complex associated protein 5], TUFM [Tu translation elongation factor, mitochondrial], TUSC3 [tumor suppressor candidate 3], TWIST1 [twist homolog 1 (Drosophila)], TXN [thioredoxin], TXNIP [thioredoxin interacting protein], TXNRD1 [thioredoxin reductase 1], TXNRD2 [thioredoxin reductase 2], TYK2 [tyrosine kinase 2], TYMP [thymidine phosphorylase], TYMS [thymidylate synthetase], TYR [tyrosinase (oculocutaneous albinism IA)], TYRO3 [TYRO3 protein tyrosine kinase], TYROBP [TYRO protein tyrosine kinase binding protein], TYRP1 [tyrosinase-related protein 1], U2AF1 [U2 small nuclear RNA auxiliary factor 1], UBA1 [ubiquitin-like modifier activating enzyme 1], UBA52 [ubiquitin A-52 residue ribosomal protein fusion product 1], UBB [ubiquitin B], UBC [ubiquitin C], UBE2A [ubiquitin-conjugating enzyme E2A (RAD6 homolog)], UBE2C [ubiquitin-conjugating enzyme E20], UBE2D2 [ubiquitin-conjugating enzyme E2D 2 (UBC4/5 homolog, yeast)], UBE2H [ubiquitin-conjugating enzyme E2H (UBC8 homolog, yeast)], UBE2I [ubiquitin-conjugating enzyme E2I (UBC9 homolog, yeast)], UBE3A [ubiquitin protein ligase E3A], UBL5 [ubiquitin-like 5], UCHL1 [ubiquitin carboxyl-terminal esterase L1 (ubiquitin thiolesterase)], UCN [urocortin], UCP1 [uncoupling protein 1 (mitochondrial, proton carrier)], UCP2 [uncoupling protein 2 (mitochondrial, proton carrier)], UCP3 [uncoupling protein 3 (mitochondrial, proton carrier)], UGT1A1 [UDP glucuronosyltransferase 1 family, polypeptide A1], UGT1A3 [UDP glucuronosyltransferase 1 family, polypeptide A3], ULK1 [unc-51-like kinase 1 (C. elegans)], UNC5A [unc-5 homolog A (C. elegans)], UNC5B [unc-5 homolog B (C. elegans)], UNC5C [unc-5 homolog C (C. elegans)], UNC5D [unc-5 homolog D (C. elegans)], UNG [uracil-DNA glycosylase], UPF3B [UPF3 regulator of nonsense transcripts homolog B (yeast)], UPK3B [uroplakin 3B], UPP2 [uridine phosphorylase 2], UQCRC1 [ubiquinol-cytochrome c reductase core protein I], USF1 [upstream transcription factor 1], USF2 [upstream transcription factor 2, c-fos interacting], USH2A [Usher syndrome 2A (autosomal recessive, mild)], USP1 [ubiquitin specific peptidase 1], USP15 [ubiquitin specific peptidase 15], USP25 [ubiquitin specific peptidase 25], USP29 [ubiquitin specific peptidase 29], USP33 [ubiquitin specific peptidase 33], USP4 [ubiquitin specific peptidase 4 (proto-oncogene)], USP5 [ubiquitin specific peptidase 5 (isopeptidase T)], USP9X [ubiquitin specific peptidase 9, X-linked], USP9Y [ubiquitin specific peptidase 9, Y-linked], UTRN [utrophin], UXT [ubiquitously-expressed transcript], VAMP7 [vesicle-associated membrane protein 7], VASP [vasodilator-stimulated phosphoprotein], VAV1 [vav 1 guanine nucleotide exchange factor], VAV2 [vav 2 guanine nucleotide exchange factor], VAX1 [ventral anterior homeobox 1], VCAM1 [vascular cell adhesion molecule 1], VCL [vinculin], VDAC1 [voltage-dependent anion channel I], VDAC2 [voltage-dependent anion channel2], VDR [vitamin D (1 [25-dihydroxyvitamin D3) receptor], VEGFA [vascular endothelial growth factor A], VEGFB [vascular endothelial growth factor B], VEGFC [vascular endothelial growth factor C], VGF [VGF nerve growth factor inducible], VHL [von Rippel-Lindau tumor suppressor], VIM [vimentin], VIP [vasoactive intestinal peptide], VIPR1 [vasoactive intestinal peptide receptor 1], VIPR2 [vasoactive intestinal peptide receptor 2], VKORC1 [vitamin K epoxide reductase complex, subunit 1], VLDLR [very low density lipoprotein receptor], VPS29 [vacuolar protein sorting 29 homolog (S. cerevisiae)], VSIG4 [V-set and immunoglobulin domain containing 4], VSX1 [visual system homeobox 1], VTN [vitronectin], VWC2 [von Willebrand factor C domain containing 2], VWF [von Willebrand factor], WAS [Wiskott-Aldrich syndrome (eczema-thrombocytopenia)], WASF1 [WAS protein family, member 1], WASF2 [WAS protein family, member 2], WASL [Wiskott-Aldrich syndrome-like], WBSCR16 [Williams-Beuren syndrome chromosome region 16], WBSCR17 [Williams-Beuren syndrome chromosome region 17], WBSCR22 [Williams Beuren syndrome chromosome region 22], WBSCR27 [Williams Beuren syndrome chromosome region 27], WBSCR28 [Williams-Beuren syndrome chromosome region 28], WDR4 [WD repeat domain 4], WEE1 [WEE1 homolog (S. pombe)], WHAMM [WAS protein homolog associated with actin, golgi membranes and microtubules], WIPF1 [WAS/WASL interacting protein family, member 1], WIPF3 [WAS/WASL interacting protein family, member 3], WNK3 [WNK lysine deficient protein kinase 3], WNT1 [wingless-type MMTV integration site family, member 1], WNT10A [wingless-type MMTV integration site family, member 10A], WNT10B [wingless-type MMTV integration site family, member 10B], WNT11 [wingless-type MMTV integration site family, member 11], WNT16 [wingless-type MMTV integration site family, member 16], WNT2 [wingless-type MMTV integration site family member 2], WNT2B [wingless-type MMTV integration site family, member 2B], WNT3 [wingless-type MMTV integration site family, member 3], WNT3A [wingless-type MMTV integration site family, member 3A], WNT4 [wingless-type MMTV integration site family, member 4], WNT5A [wingless-type MMTV integration site family, member SA], WNTSB [wingless-type MMTV integration site family, member 5B], WNT6 [wingless-type MMTV integration site family, member 6], WNT7A [wingless-type MMTV integration site family, member 7A], WNT7B [wingless-type MMTV integration site family, member 7B], WNT8A [wingless-type MMTV integration site family, member 8A], WNT8B [wingless-type MMTV integration site family, member 8B], WNT9A [wingless-type MMTV integration site family, member 9A], WNT9B [wingless-type MMTV integration site family, member 9B], WRB [tryptophan rich basic protein], WRN [Werner syndrome, RecQ helicase-like], WT1 [Wilms tumor 1], XBP1 [X-box binding protein 1], XCL1 [chemokine (C motif) ligand 1], XDH [xanthine dehydrogenase], XIAP [X-linked inhibitor of apoptosis], XIRP2 [xin actin-binding repeat containing 2], XPC [xeroderma pigmentosum, complementation group C], XRCC1 [X-ray repair complementing defective repair in Chinese hamster cells 1], XRCC5 [X-ray repair complementing defective repair in Chinese hamster cells 5 (double-strand-break rejoining)], XRCC6 [X-ray repair complementing defective repair in Chinese hamster cells 6], XRN1 [5′-3′ exoribonuclease 1], YBX1 [Y box binding protein 1], YWHAB [tyrosine 3-monooxygenase/tryptophan 5-monooxygenase activation protein, beta polypeptide], YWHAE [tyrosine 3-monooxygenase/tryptophan 5-monooxygenase activation protein, epsilon polypeptide], YWHAG [tyrosine 3-monooxygenase/tryptophan 5-monooxygenase activation protein, gamma polypeptide], YWHAQ [tyrosine 3-monooxygenase/tryptophan 5-monooxygenase activation protein, theta polypeptide], YWHAZ [tyrosine 3-monooxygenase/tryptophan 5-monooxygenase activation protein, zeta polypeptide], ZAP70 [zeta-chain (TCR) associated protein kinase 70 kDa], ZBTB16 [zinc finger and BTB domain containing 16], ZBTB33 [zinc finger and BTB domain containing 33], ZC3H12A [zinc finger CCCH-type containing 12A], ZEB1 [zinc finger E-box binding homeobox 1], ZEB2 [zinc finger E-box binding homeobox 2], ZFP161 [zinc finger protein 161 homolog (mouse)], ZFP36 [zinc finger protein 36, C3H type, homolog (mouse)], ZFP42 [zinc finger protein 42 homolog (mouse)], ZFP57 [zinc finger protein 57 homolog (mouse)], ZFPM1 [zinc finger protein, multitype 1], ZFPM2 [zinc finger protein, multitype 2], ZFY [zinc finger protein, Y-linked], ZFYVE9 [zinc finger, FYVE domain containing 9], ZIC1 [Zic family member 1 (odd-paired homolog, Drosophila)], ZIC2 [Zic family member 2 (odd-paired homolog, Drosophila)], ZIC3 [Zic family member 3 (odd-paired homolog, Drosophila)], ZMPSTE24 [zinc metallopeptidase (STE24 homolog, S. cerevisiae)], ZNF148 [zinc finger protein 148], ZNF184 [zinc finger protein 184], ZNF225 [zinc finger protein 225], ZNF256 [zinc finger protein 256], ZNF333 [zinc finger protein 333], ZNF385B [zinc finger protein 385B], ZNF44 [zinc finger protein44], ZNF521 [zinc finger protein 521], ZNF673 [zinc finger family member 673], ZNF79 [zinc finger protein 79], ZNF84 [zinc finger protein 84], ZW10 [ZW10, kinetochore associated, homolog (Drosophila)], and ZYX [zyxin].

Other inducible systems are contemplated such as, but not limited to, regulation by heavy-metals [Mayo K E et al., Cell 1982, 29:99-108; Searle P F et al., Mol Cell Biol 1985, 5:1480-1489 and Brinster R L et al., Nature (London) 1982, 296:39-42], steroid hormones [Hynes N E et al., Proc Natl Acad Sci USA 1981, 78:2038-2042; Klock G et al., Nature (London) 1987, 329:734-736 and Lee F et al., Nature (London) 1981, 294:228-232.], heat shock [Nouer L: Heat Shock Response. Boca Raton, Fla.: CRC; 1991] and other reagents have been developed [Mullick A, Massie B: Transcription, translation and the control of gene expression. In Encyclopedia of Cell Technology Edited by: Speir R E. Wiley; 2000:1140-1164 and Fussenegger M, . Biotechnol Prog 2001, 17:1-51]. However, there are limitations with these inducible mammalian promoters such as “leakiness” of the “off” state and pleiotropic effects of inducers (heat shock, heavy metals, glucocorticoids etc.). The use of insect hormones (ecdysone) has been proposed in an attempt to reduce the interference with cellular processes in mammalian cells [No D et al., Proc Natl Acad Sci USA 1996, 93:3346-3351]. Another elegant system uses rapamycin as the inducer [Rivera V M et al., Nat Med 1996, 2:1028-1032] but the role of rapamycin as an immunosuppressant was a major limitation to its use in vivo and therefore it was necessary to find a biologically inert compound [Saez E et al., Proc Natl Acad Sci USA 2000, 97:14512-14517] for the control of gene expression.

The present invention also encompasses nucleic acid encoding the polypeptides of the present invention. The nucleic acid may comprise a promoter, advantageously human Synapsin I promoter (hSyn). In a particularly advantageous embodiment, the nucleic acid may be packaged into an adeno associated viral vector (AAV).

Also contemplated by the present invention are recombinant vectors and recombinant adenoviruses that may comprise subviral particles from more than one adenovirus serotype. For example, it is known that adenovirus vectors may display an altered tropism for specific tissues or cell types (Havenga, M. J. E. et al., 2002), and therefore, mixing and matching of different adenoviral capsids, i.e., fiber, or penton proteins from various adenoviral serotypes may be advantageous. Modification of the adenoviral capsids, including fiber and penton may result in an adenoviral vector with a tropism that is different from the unmodified adenovirus. Adenovirus vectors that are modified and optimized in their ability to infect target cells may allow for a significant reduction in the therapeutic or prophylactic dose, resulting in reduced local and disseminated toxicity.

Viral vector gene delivery systems are commonly used in gene transfer and gene therapy applications. Different viral vector systems have their own unique advantages and disadvantages. Viral vectors that may be used to express the pathogen-derived ligand of the present invention include but are not limited to adenoviral vectors, adeno-associated viral vectors, alphavirus vectors, herpes simplex viral vectors, and retroviral vectors, described in more detail below.

Additional general features of adenoviruses are such that the biology of the adenovirus is characterized in detail; the adenovirus is not associated with severe human pathology; the adenovirus is extremely efficient in introducing its DNA into the host cell; the adenovirus may infect a wide variety of cells and has a broad host range; the adenovirus may be produced in large quantities with relative ease; and the adenovirus may be rendered replication defective and/or non-replicating by deletions in the early region 1 (“E1”) of the viral genome.

Adenovirus is a non-enveloped DNA virus. The genome of adenovirus is a linear double-stranded DNA molecule of approximately 36,000 base pairs (“bp”) with a 55-kDa terminal protein covalently bound to the 5′-terminus of each strand. The adenovirus DNA contains identical inverted terminal repeats (“ITRs”) of about 100 bp, with the exact length depending on the serotype. The viral origins of replication are located within the ITRs exactly at the genome ends. DNA synthesis occurs in two stages. First, replication proceeds by strand displacement, generating a daughter duplex molecule and a parental displaced strand. The displaced strand is single stranded and may form a “panhandle” intermediate, which allows replication initiation and generation of a daughter duplex molecule. Alternatively, replication may proceed from both ends of the genome simultaneously, obviating the requirement to form the panhandle structure.

During the productive infection cycle, the viral genes are expressed in two phases: the early phase, which is the period up to viral DNA replication, and the late phase, which coincides with the initiation of viral DNA replication. During the early phase, only the early gene products, encoded by regions E1, E2, E3 and E4, are expressed, which carry out a number of functions that prepare the cell for synthesis of viral structural proteins (Berk, A. J., 1986). During the late phase, the late viral gene products are expressed in addition to the early gene products and host cell DNA and protein synthesis are shut off. Consequently, the cell becomes dedicated to the production of viral DNA and of viral structural proteins (Tooze, J., 1981).

The E1 region of adenovirus is the first region of adenovirus expressed after infection of the target cell. This region consists of two transcriptional units, the E1A and E1B genes, both of which are required for oncogenic transformation of primary (embryonal) rodent cultures. The main functions of the E1A gene products are to induce quiescent cells to enter the cell cycle and resume cellular DNA synthesis, and to transcriptionally activate the E1B gene and the other early regions (E2, E3 and E4) of the viral genome. Transfection of primary cells with the E1A gene alone may induce unlimited proliferation (immortalization), but does not result in complete transformation. However, expression of E1A, in most cases, results in induction of programmed cell death (apoptosis), and only occasionally is immortalization obtained (Jochemsen et al., 1987). Co-expression of the E1B gene is required to prevent induction of apoptosis and for complete morphological transformation to occur. In established immortal cell lines, high-level expression of E1A may cause complete transformation in the absence of E1B (Roberts, B. E. et al., 1985).

The E1B encoded proteins assist E1A in redirecting the cellular functions to allow viral replication. The E1B 55 kD and E4 33 kD proteins, which form a complex that is essentially localized in the nucleus, function in inhibiting the synthesis of host proteins and in facilitating the expression of viral genes. Their main influence is to establish selective transport of viral mRNAs from the nucleus to the cytoplasm, concomitantly with the onset of the late phase of infection. The E1B 21 kD protein is important for correct temporal control of the productive infection cycle, thereby preventing premature death of the host cell before the virus life cycle has been completed. Mutant viruses incapable of expressing the E1B 21 kD gene product exhibit a shortened infection cycle that is accompanied by excessive degradation of host cell chromosomal DNA (deg-phenotype) and in an enhanced cytopathic effect (cyt-phenotype; Telling et al., 1994). The deg and cyt phenotypes are suppressed when in addition the E1A gene is mutated, indicating that these phenotypes are a function of E1A (White, E. et al., 1988). Furthermore, the E1B 21 kDa protein slows down the rate by which E1A switches on the other viral genes. It is not yet known by which mechanisms EIB 21 kD quenches these E1A dependent functions.

In contrast to, for example, retroviruses, adenoviruses do not efficiently integrate into the host cell's genome, are able to infect non-dividing cells, and are able to efficiently transfer recombinant genes in vivo (Brody et al., 1994). These features make adenoviruses attractive candidates for in vivo gene transfer of, for example, an antigen or immunogen of interest into cells, tissues or subjects in need thereof.

Adenovirus vectors containing multiple deletions are preferred to both increase the carrying capacity of the vector and reduce the likelihood of recombination to generate replication competent adenovirus (RCA). Where the adenovirus contains multiple deletions, it is not necessary that each of the deletions, if present alone, would result in a replication defective and/or non-replicating adenovirus. As long as one of the deletions renders the adenovirus replication defective or non-replicating, the additional deletions may be included for other purposes, e.g., to increase the carrying capacity of the adenovirus genome for heterologous nucleotide sequences. Preferably, more than one of the deletions prevents the expression of a functional protein and renders the adenovirus replication defective and/or non-replicating and/or attenuated. More preferably, all of the deletions are deletions that would render the adenovirus replication-defective and/or non-replicating and/or attenuated. However, the invention also encompasses adenovirus and adenovirus vectors that are replication competent and/or wild-type, i.e. comprises all of the adenoviral genes necessary for infection and replication in a subject.

Embodiments of the invention employing adenovirus recombinants may include E1-defective or deleted, or E3-defective or deleted, or E4-defective or deleted or adenovirus vectors comprising deletions of E1 and E3, or E1 and E4, or E3 and E4, or E1, E3, and E4 deleted, or the “gutless” adenovirus vector in which all viral genes are deleted. The adenovirus vectors may comprise mutations in E1, E3, or E4 genes, or deletions in these or all adenoviral genes. The E1 mutation raises the safety margin of the vector because E1-defective adenovirus mutants are said to be replication-defective and/or non-replicating in non-permissive cells, and are, at the very least, highly attenuated. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. The present invention comprehends adenovirus vectors of any serotype or serogroup that are deleted or mutated in E1, or E3, or E4, or E1 and E3, or E1 and E4. Deletion or mutation of these adenoviral genes result in impaired or substantially complete loss of activity of these proteins.

The “gutless” adenovirus vector is another type of vector in the adenovirus vector family. Its replication requires a helper virus and a special human 293 cell line expressing both E1a and Cre, a condition that does not exist in a natural environment; the vector is deprived of all viral genes, thus the vector as a vaccine carrier is non-immunogenic and may be inoculated multiple times for re-vaccination. The “gutless” adenovirus vector also contains 36 kb space for accommodating antigen or immunogen(s) of interest, thus allowing co-delivery of a large number of antigen or immunogens into cells.

Adeno-associated virus (AAV) is a single-stranded DNA parvovirus which is endogenous to the human population. Although capable of productive infection in cells from a variety of species, AAV is a dependovirus, requiring helper functions from either adenovirus or herpes virus for its own replication. In the absence of helper functions from either of these helper viruses, AAV will infect cells, uncoat in the nucleus, and integrate its genome into the host chromosome, but will not replicate or produce new viral particles.

The genome of AAV has been cloned into bacterial plasmids and is well characterized. The viral genome consists of 4682 bases which include two terminal repeats of 145 bases each. These terminal repeats serve as origins of DNA replication for the virus. Some investigators have also proposed that they have enhancer functions. The rest of the genome is divided into two functional domains. The left portion of the genome codes for the rep functions which regulate viral DNA replication and vital gene expression. The right side of the vital genome contains the cap genes that encode the structural capsid proteins VP1, VP2 and VP3. The proteins encoded by both the rep and cap genes function in trans during productive AAV replication.

AAV is considered an ideal candidate for use as a transducing vector, and it has been used in this manner. Such AAV transducing vectors comprise sufficient cis-acting functions to replicate in the presence of adenovirus or herpes virus helper functions provided in trans. Recombinant AAV (rAAV) have been constructed in a number of laboratories and have been used to carry exogenous genes into cells of a variety of lineages. In these vectors, the AAV cap and/or rep genes are deleted from the viral genome and replaced with a DNA segment of choice. Current vectors may accommodate up to 4300 bases of inserted DNA.

To produce rAAV, plasmids containing the desired vital construct are transfected into adenovirus-infected cells. In addition, a second helper plasmid is cotransfected into these cells to provide the AAV rep and cap genes which are obligatory for replication and packaging of the recombinant viral construct. Under these conditions, the rep and cap proteins of AAV act in trans to stimulate replication and packaging of the rAAV construct. Three days after transfection, rAAV is harvested from the cells along with adenovirus. The contaminating adenovirus is then inactivated by heat treatment.

Herpes Simplex Virus 1 (HSV-1) is an enveloped, double-stranded DNA virus with a genome of 153 kb encoding more than 80 genes. Its wide host range is due to the binding of viral envelope glycoproteins to the extracellular heparin sulphate molecules found in cell membranes (WuDunn & Spear, 1989). Internalization of the virus then requires envelope glycoprotein gD and fibroblast growth factor receptor (Kaner, 1990). HSV is able to infect cells lytically or may establish latency. HSV vectors have been used to infect a wide variety of cell types (Lowenstein, 1994; Huard, 1995; Miyanohara, 1992; Liu, 1996; Goya, 1998).

There are two types of HSV vectors, called the recombinant HSV vectors and the amplicon vectors. Recombinant HSV vectors are generated by the insertion of transcription units directly into the HSV genome, through homologous recombination events. The amplicon vectors are based on plasmids bearing the transcription unit of choice, an origin of replication, and a packaging signal.

HSV vectors have the obvious advantages of a large capacity for insertion of foreign genes, the capacity to establish latency in neurons, a wide host range, and the ability to confer transgene expression to the CNS for up to 18 months (Carpenter & Stevens, 1996).

Retroviruses are enveloped single-stranded RNA viruses, which have been widely used in gene transfer protocols. Retroviruses have a diploid genome of about 7-10 kb, composed of four gene regions termed gag, pro, pol and env. These gene regions encode for structural capsid proteins, viral protease, integrase and viral reverse transcriptase, and envelope glycoproteins, respectively. The genome also has a packaging signal and cis-acting sequences, termed long-terminal repeats (LTRs), at each end, which have a role in transcriptional control and integration.

The most commonly used retroviral vectors are based on the Moloney murine leukaemia virus (Mo-MLV) and have varying cellular tropisms, depending on the receptor binding surface domain of the envelope glycoprotein.

Recombinant retroviral vectors are deleted from all retroviral genes, which are replaced with marker or therapeutic genes, or both. To propagate recombinant retroviruses, it is necessary to provide the viral genes, gag, pol and env in trans.

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Alphaviruses, including the prototype Sindbis virus (SIN), Semliki Forest virus (SFV), and Venezuelan equine encephalitis virus (VEE), constitute a group of enveloped viruses containing plus-stranded RNA genomes within icosahedral capsids.

The viral vectors of the present invention are useful for the delivery of nucleic acids expressing antigens or immunogens to cells both in vitro and in vivo. In particular, the inventive vectors may be advantageously employed to deliver or transfer nucleic acids to cells, more preferably mammalian cells. Nucleic acids of interest include nucleic acids encoding peptides and proteins, preferably therapeutic (e.g., for medical or veterinary uses) or immunogenic (e.g., for vaccines) peptides or proteins.

Preferably, the codons encoding the antigen or immunogen of interest are “optimized” codons, i.e., the codons are those that appear frequently in, e.g.., highly expressed genes in the subject's species, instead of those codons that are frequently used by, for example, an influenza virus. Such codon usage provides for efficient expression of the antigen or immunogen in animal cells. In other embodiments, for example, when the antigen or immunogen of interest is expressed in bacteria, yeast or another expression system, the codon usage pattern is altered to represent the codon bias for highly expressed genes in the organism in which the antigen or immunogen is being expressed. Codon usage patterns are known in the literature for highly expressed genes of many species (e.g., Nakamura et al., 1996; Wang et al., 1998; McEwan et al. 1998).

As a further alternative, the viral vectors may be used to infect a cell in culture to express a desired gene product, e.g., to produce a protein or peptide of interest. Preferably, the protein or peptide is secreted into the medium and may be purified therefrom using routine techniques known in the art. Signal peptide sequences that direct extracellular secretion of proteins are known in the art and nucleotide sequences encoding the same may be operably linked to the nucleotide sequence encoding the peptide or protein of interest by routine techniques known in the art. Alternatively, the cells may be lysed and the expressed recombinant protein may be purified from the cell lysate. Preferably, the cell is an animal cell, more preferably a mammalian cell. Also preferred are cells that are competent for transduction by particular viral vectors of interest. Such cells include PER.C6 cells, 911 cells, and HEK293 cells.

A culture medium for culturing host cells includes a medium commonly used for tissue culture, such as M199-earle base, Eagle MEM (E-MEM), Dulbecco MEM (DMEM), SC-UCM102, UP-SFM (GIBCO BRL), EX-CELL302 (Nichirei), EX-CELL293-S(Nichirei), TFBM-01 (Nichirei), ASF104, among others. Suitable culture media for specific cell types may be found at the American Type Culture Collection (ATCC) or the European Collection of Cell Cultures (ECACC). Culture media may be supplemented with amino acids such as L-glutamine, salts, anti-fungal or anti-bacterial agents such as Fungizone®, penicillin-streptomycin, animal serum, and the like. The cell culture medium may optionally be serum-free.

The present invention also relates to cell lines or transgenic animals which are capable of expressing or overexpressing LITEs or at least one agent useful in the present invention. Preferably the cell line or animal expresses or overexpresses one or more LITEs.

The transgenic animal is typically a vertebrate, more preferably a rodent, such as a rat or a mouse, but also includes other mammals such as human, goat, pig or cow etc.

Such transgenic animals are useful as animal models of disease and in screening assays for new useful compounds. By specifically expressing one or more polypeptides, as defined above, the effect of such polypeptides on the development of disease may be studied. Furthermore, therapies including gene therapy and various drugs may be tested on transgenic animals. Methods for the production of transgenic animals are known in the art. For example, there are several possible routes for the introduction of genes into embryos. These include (i) direct transfection or retroviral infection of embryonic stem cells followed by introduction of these cells into an embryo at the blastocyst stage of development; (ii) retroviral infection of early embryos; and (iii) direct microinjection of DNA into zygotes or early embryo cells. The gene and/or transgene may also include genetic regulatory elements and/or structural elements known in the art. A type of target cell for transgene introduction is the embryonic stem cell (ES). ES cells may be obtained from pre-implantation embryos cultured in vitro and fused with embryos (Evans et al., 1981, Nature 292:154-156; Bradley et al., 1984, Nature 309:255-258; Gossler et al., 1986, Proc. Natl. Acad. Sci. USA 83:9065-9069; and Robertson et al., 1986 Nature 322:445-448). Transgenes may be efficiently introduced into the ES cells by a variety of standard techniques such as DNA transfection, microinjection, or by retrovirus-mediated transduction. The resultant transformed ES cells may thereafter be combined with blastocysts from a non-human animal. The introduced ES cells thereafter colonize the embryo and contribute to the germ line of the resulting chimeric animal (Jaenisch, 1988, Science 240: 1468-1474).

LITEs may also offer valuable temporal precision in vivo. LITEs may be used to alter gene expression during a particular stage of development, for example, by repressing a particular apoptosis gene only during a particular stage of C. elegans growth. LITEs may be used to time a genetic cue to a particular experimental window. For example, genes implicated in learning may be overexpressed or repressed only during the learning stimulus in a precise region of the intact rodent or primate brain. Further, LITEs may be used to induce gene expression changes only during particular stages of disease development. For example, an oncogene may be overexpressed only once a tumor reaches a particular size or metastatic stage. Conversely, proteins suspected in the development of Alzheimer's may be knocked down only at defined time points in the animal's life and within a particular brain region. Although these examples do not exhaustively list the potential applications of the LITE system, they highlight some of the areas in which LITEs may be a powerful technology.

Therapeutic or diagnostic compositions of the invention are administered to an individual in amounts sufficient to treat or diagnose disorders. The effective amount may vary according to a variety of factors such as the individual's condition, weight, sex and age. Other factors include the mode of administration.

The pharmaceutical compositions may be provided to the individual by a variety of routes such as subcutaneous, topical, oral and intramuscular.

Compounds identified according to the methods disclosed herein may be used alone at appropriate dosages. Alternatively, co-administration or sequential administration of other agents may be desirable.

The present invention also has the objective of providing suitable topical, oral, systemic and parenteral pharmaceutical formulations for use in the novel methods of treatment of the present invention. The compositions containing compounds identified according to this invention as the active ingredient may be administered in a wide variety of therapeutic dosage forms in conventional vehicles for administration. For example, the compounds may be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

Advantageously, compounds of the present invention may be administered in a single daily dose, or the total daily dosage may be administered in divided doses of two, three or four times daily. Furthermore, compounds for the present invention may be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents may be administered concurrently, or they each may be administered at separately staggered times.

The dosage regimen utilizing the compounds of the present invention is selected in accordance with a variety of factors including type, species, age, weight, sex and medical condition of the patient; the severity of the condition to be treated; the route of administration; the renal, hepatic and cardiovascular function of the one patient; and the particular compound thereof employed. A physician of ordinary skill may readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

Although the present invention and its advantages have been described in detail, it should be understood that various changes, substitutions and alterations may be made herein without departing from the spirit and scope of the invention as defined in the appended claims.

The present invention will be further illustrated in the following Examples which are given for illustration purposes only and are not intended to limit the invention in any way.

### EXAMPLES

### Example 1

The ability to directly modulate gene expression from the endogenous mammalian genome is critical for elucidating normal gene function and disease mechanism. Advances that further refine the spatial and temporal control of gene expression within cell populations have the potential to expand the utility of gene modulation. Applicants previously developed transcription activator-like effectors (TALEs) from Xanthamonas oryze to enable the rapid design and construction of site-specific DNA binding proteins. Applicants developed a set of molecular tools for enabling light-regulated gene expression in the endogenous mammalian genome. The system consists of engineered artificial transcription factors linked to light-sensitive dimerizing protein domains from Arabidopsis thaliana. The system responds to light in the range of 450 nm-500 nm and is capable of inducing a significant increase in the expression of pluripotency factors after stimulation with light at an intensity of 6.2 mW/cm² in mammalian cells. Applicants are developing tools for the targeting of a wide range of genes. Applicants believe that a toolbox for the light-mediated control of gene expression would complement the existing optogenetic methods and may in the future help elucidate the timing-, cell type- and concentration dependent role of specific genes in the brain.

The ability to directly modulate gene expression from the endogenous mammalian genome is critical for elucidating normal gene function and disease mechanisms. Applicants present the development of a set of molecular tools for enabling light-regulated gene expression in the endogenous mammalian genome. This system consists of a transcription activator like effector (TALE) and the activation domain VP64 linked to the light-sensitive dimerizing protein domains cryptochrome 2 (CRY2) and CIB1 from Arabidopsis thaliana. Applicants show that blue-light stimulation of HEK293FT and Neuro-2a cells transfected with these LITE constructs designed to target the promoter region of KLF4 and Neurog2 results in a significant increase in target expression, demonstrating the functionality of TALE-based optical gene expression modulation technology.

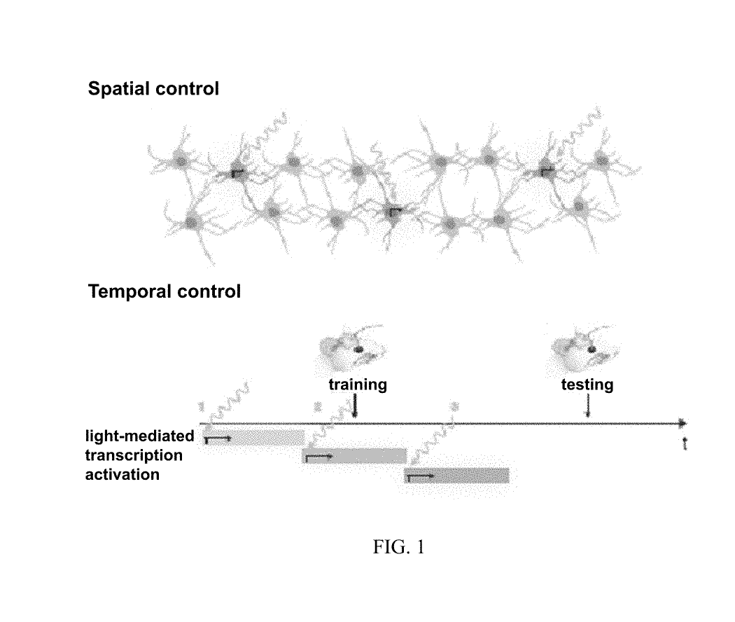

FIG. 1 shows a schematic depicting the need for spatial and temporal precision.

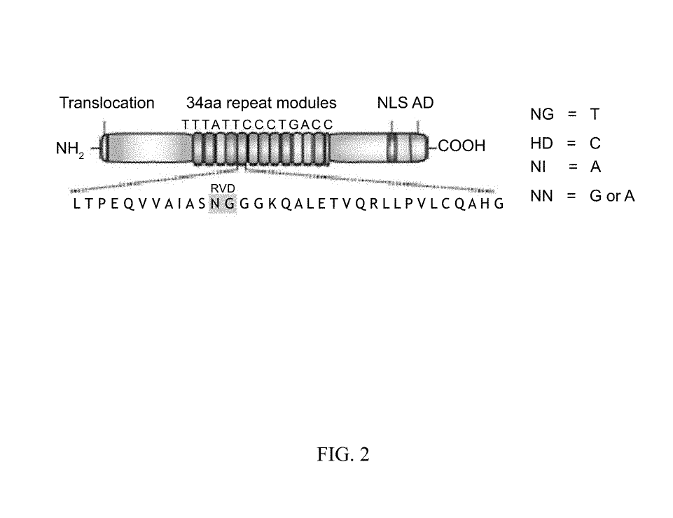

FIG. 2 shows transcription activator like effectors (TALEs). TALEs consist of 34 aa repeats at the core of their sequence. Each repeat corresponds to a base in the target DNA that is bound by the TALE. Repeats differ only by 2 variable amino acids at positions 12 and 13. The code of this correspondence has been elucidated (Boch, J et al. , Science, 2009 and Moscou, M et al. , Science, 2009) and is shown in this figure. Applicants developed a method for the synthesis of designer TALEs incorporating this code and capable of binding a sequence of choice within the genome (Zhang, F et al. , Nature Biotechnology, 2011).

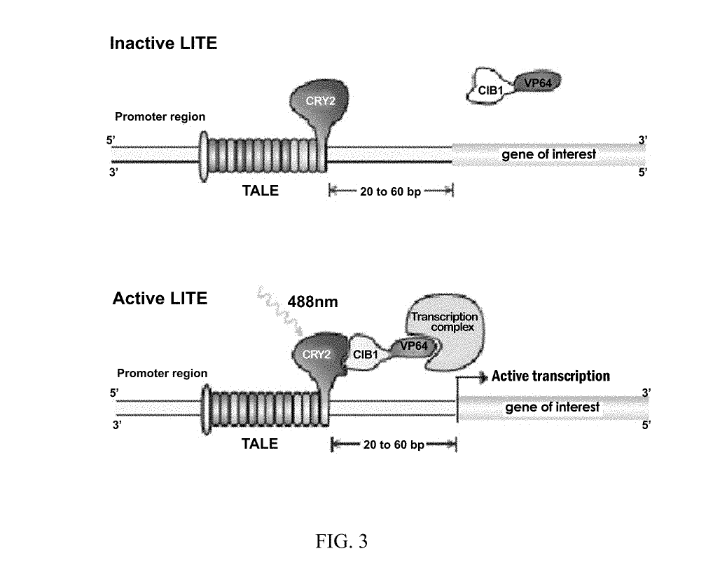

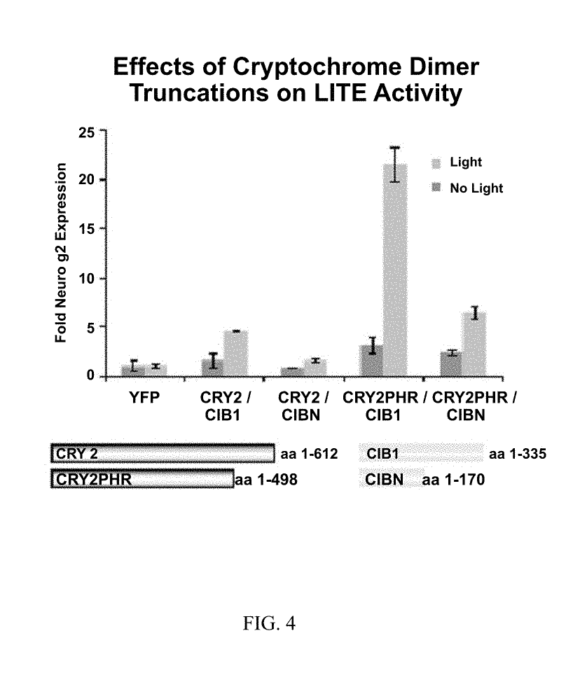

FIG. 3 depicts a design of a LITE: TALE/Cryptochrome transcriptional activation. Each LITE is a two-component system which may comprise a TALE fused to CRY2 and the cryptochrome binding partner CIB1 fused to VP64, a transcription activor. In the inactive state, the TALE localizes its fused CRY2 domain to the promoter region of the gene of interest. At this point, CIB1 is unable to bind CRY2, leaving the CIB1-VP64 unbound in the nuclear space. Upon stimulation with 488 nm (blue) light, CRY2 undergoes a conformational change, revealing its CIB1 binding site (Liu, H et al. , Science, 2008). Rapid binding of CIB1 results in recruitment of the fused VP64 domain, which induces transcription of the target gene.

FIG. 4 depicts effects of cryptochrome dimer truncations on LITE activity. Truncations known to alter the activity of CRY2 and CIB1 ( ) were compared against the full length proteins. A LITE targeted to the promoter of Neurog2 was tested in Neuro-2a cells for each combination of domains. Following stimulation with 488 nm light, transcript levels of Neurog2 were quantified using qPCR for stimulated and unstimulated samples.

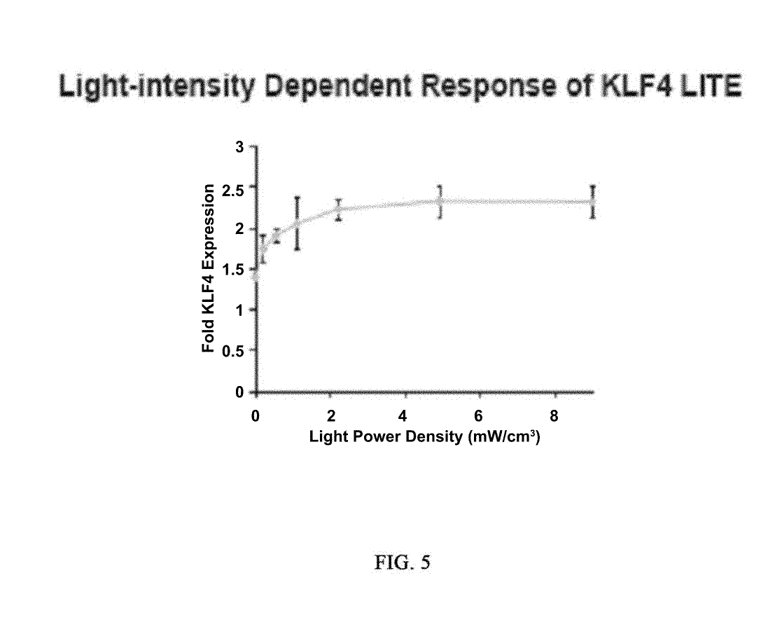

FIG. 5 depicts a light-intensity dependent response of KLF4 LITE.

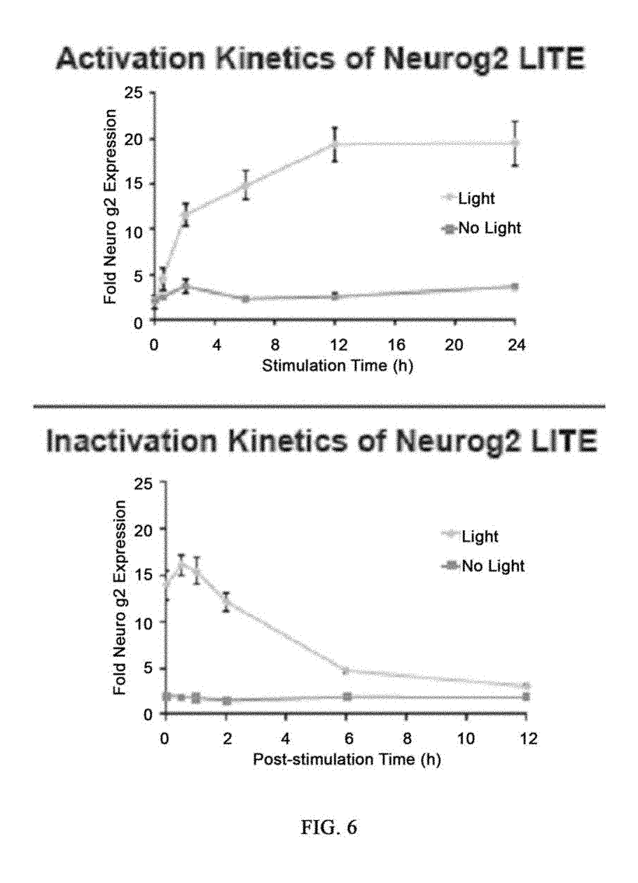

FIG. 6 depicts activation kinetics of Neurog2 LITE and inactivation kinetics of Neurog2 LITE.

### Example 2

Normal gene expression is a dynamic process with carefully orchestrated temporal and spatial components, the precision of which are necessary for normal development, homeostasis, and advancement of the organism. In turn, the dysregulation of required gene expression patterns, either by increased, decreased, or altered function of a gene or set of genes, has been linked to a wide array of pathologies. Technologies capable of modulating gene expression in a spatiotemporally precise fashion will enable the elucidation of the genetic cues responsible for normal biological processes and disease mechanisms. To address this technological need, Applicants developed light-inducible transcriptional effectors (LITEs), which provide light-mediated control of endogenous gene expression.

Inducible gene expression systems have typically been designed to allow for chemically inducible activation of an inserted open reading frame or shRNA sequence, resulting in gene overexpression or repression, respectively. Disadvantages of using open reading frames for overexpression include loss of splice variation and limitation of gene size. Gene repression via RNA interference, despite its transformative power in human biology, may be hindered by complicated off-target effects. Certain inducible systems including estrogen, ecdysone, and FKBP12/FRAP based systems are known to activate off-target endogenous genes. The potentially deleterious effects of long-term antibiotic treatment may complicate the use of tetracycline transactivator (TET) based systems. In vivo, the temporal precision of these chemically inducible systems is dependent upon the kinetics of inducing agent uptake and elimination. Further, because inducing agents are generally delivered systemically, the spatial precision of such systems is bounded by the precision of exogenous vector delivery.

In response to these limitations, LITEs are designed to modulate expression of individual endogenous genes in a temporally and spatially precise manner. Each LITE is a two component system consisting of a customized DNA-binding transcription activator like effector (TALE) protein, a light-responsive crytochrome heterodimer from Arabadopsis thaliana, and a transcriptional activation/repression domain. The TALE is designed to bind to the promoter sequence of the gene of interest. The TALE protein is fused to one half of the cryptochrome heterodimer (cryptochrome-2 or CIB1), while the remaining cryptochrome partner is fused to a transcriptional effector domain. Effector domains may be either activators, such as VP16, VP64, or p65, or repressors, such as KRAB, EnR, or SID. In a LITE's unstimulated state, the TALE-cryptochrome2 protein localizes to the promoter of the gene of interest, but is not bound to the CIB1-effector protein. Upon stimulation of a LITE with blue spectrum light, cryptochrome-2 becomes activated, undergoes a conformational change, and reveals its binding domain. CIB1, in turn, binds to cryptochrome-2 resulting in localization of the effector domain to the promoter region of the gene of interest and initiating gene overexpression or silencing.

Gene targeting in a LITE is achieved via the specificity of customized TALE DNA binding proteins. A target sequence in the promoter region of the gene of interest is selected and a TALE customized to this sequence is designed. The central portion of the TALE consists of tandem repeats 34 amino acids in length. Although the sequences of these repeats are nearly identical, the 12th and 13th amino acids (termed repeat variable diresidues) of each repeat vary, determining the nucleotide-binding specificity of each repeat. Thus, by synthesizing a construct with the appropriate ordering of TALE monomer repeats, a DNA binding protein specific to the target promoter sequence is created.

Light responsiveness of a LITE is achieved via the activation and binding of cryptochrome-2 and CIB1. As mentioned above, blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a LITE system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a LITE stimulated region, allowing for greater precision than vector delivery alone may offer.

The modularity of the LITE system allows for any number of effector domains to be employed for transcriptional modulation. Thus, activator and repressor domains may be selected on the basis of species, strength, mechanism, duration, size, or any number of other parameters.

Applicants next present two prototypical manifestations of the LITE system. The first example is a LITE designed to activate transcription of the mouse gene NEUROG2. The sequence TGAATGATGATAATACGA (SEQ ID NO: 27), located in the upstream promoter region of mouse NEUROG2, was selected as the target and a TALE was designed and synthesized to match this sequence. The TALE sequence was linked to the sequence for cryptochrome-2 via a nuclear localization signal (amino acids: SPKKKRKVEAS (SEQ ID NO: 28)) to facilitate transport of the protein from the cytosol to the nuclear space. A second vector was synthesized comprising the CIB1 domain linked to the transcriptional activator domain VP64 using the same nuclear localization signal. This second vector, also a GFP sequence, is separated from the CIB1-VP64 fusion sequence by a 2A translational skip signal. Expression of each construct was driven by a ubiquitous, constitutive promoter (CMV or EF1-α). Mouse neuroblastoma cells from the Neuro 2A cell line were co-transfected with the two vectors. After incubation to allow for vector expression, samples were stimulated by periodic pulsed blue light from an array of 488 nm LEDs. Unstimulated co-transfected samples and samples transfected only with the fluorescent reporter YFP were used as controls. At the end of each experiment, mRNA was purified from the samples analyzed via qPCR.

Truncated versions of cryptochrome-2 and CIB1 were cloned and tested in combination with the full-length versions of cryptochrome-2 and CIB1 in order to determine the effectiveness of each heterodimer pair. The combination of the CRY2PHR domain, consisting of the conserved photoresponsive region of the cryptochrome-2 protein, and the full-length version of CIB1 resulted in the highest upregulation of Neurog2 mRNA levels (˜22 fold over YFP samples and ˜7 fold over unstimulated co-transfected samples). The combination of full-length cryptochrome-2 (CRY2) with full-length CIB1 resulted in a lower absolute activation level (˜4.6 fold over YFP), but also a lower baseline activation (˜1.6 fold over YFP for unstimulated co-transfected samples). These cryptochrome protein pairings may be selected for particular uses depending on absolute level of induction required and the necessity to minimize baseline “leakiness” of the LITE system.

Speed of activation and reversibility are critical design parameters for the LITE system. To characterize the kinetics of the LITE system, constructs consisting of the Neurog2 TALE-CRY2PHR and CIB1-VP64 version of the system were tested to determine its activation and inactivation speed. Samples were stimulated for as little as 0.5 h to as long as 24 h before extraction. Upregulation of Neurog2 expression was observed at the shortest, 0.5 h, time point (˜5 fold vs YFP samples). Neurog2 expression peaked at 12 h of stimulation (˜19 fold vs YFP samples). Inactivation kinetics were analyzed by stimulating co-transfected samples for 6 h, at which time stimulation was stopped, and samples were kept in culture for 0 to 12 h to allow for mRNA degradation. Neurog2 mRNA levels peaked at 0.5 h after the end of stimulation (˜16 fold vs. YFP samples), after which the levels degraded with an ˜3 h half-life before returning to near baseline levels by 12 h.

The second prototypical example is a LITE designed to activate transcription of the human gene KLF4. The sequence TTCTTACTTATAAC (SEQ ID NO: 29), located in the upstream promoter region of human KLF4, was selected as the target and a TALE was designed and synthesized to match this sequence. The TALE sequence was linked to the sequence for CRY2PHR via a nuclear localization signal (amino acids: SPKKKRKVEAS (SEQ ID NO: 28)). The identical CIB1-VP64 activator protein described above was also used in this manifestation of the LITE system. Human embryonal kidney cells from the HEK293FT cell line were co-transfected with the two vectors. After incubation to allow for vector expression, samples were stimulated by periodic pulsed blue light from an array of 488 nm LEDs. Unstimulated co-transfected samples and samples transfected only with the fluorescent reporter YFP were used as controls. At the end of each experiment, mRNA was purified from the samples analyzed via qPCR.

The light-intensity response of the LITE system was tested by stimulating samples with increased light power (0-9 mW/cm²). Upregulation of KLF4 mRNA levels was observed for stimulation as low as 0.2 mW/cm². KLF4 upregulation became saturated at 5 mW/cm² (2.3 fold vs. YFP samples). Cell viability tests were also performed for powers up to 9 mW/cm² and showed >98% cell viability. Similarly, the KLF4 LITE response to varying duty cycles of stimulation was tested (1.6-100%). No difference in KLF4 activation was observed between different duty cycles indicating that a stimulation paradigm of as low as 0.25 sec every 15 sec should result in maximal activation.

There are potential applications for which LITEs represent an advantageous choice for gene expression control. There exist a number of in vitro applications for which LITEs are particularly attractive. In all these cases, LITEs have the advantage of inducing endogenous gene expression with the potential for correct splice variant expression.

Because LITE activation is photoinducible, spatially defined light patterns, created via masking or rasterized laser scanning, may be used to alter expression levels in a confined subset of cells. For example, by overexpressing or silencing an intercellular signaling molecule only in a spatially constrained set of cells, the response of nearby cells relative to their distance from the stimulation site may help elucidate the spatial characteristics of cell non-autonomous processes. Additionally, recent advances in cell reprogramming biology have shown that overexpression of sets of transcription factors may be utilized to transform one cell type, such as fibroblasts, into another cell type, such as neurons or cardiomyocytes. Further, the correct spatial distribution of cell types within tissues is critical for proper organotypic function. Overexpression of reprogramming factors using LITEs may be employed to reprogram multiple cell lineages in a spatially precise manner for tissue engineering applications.

The rapid transcriptional response and endogenous targeting of LITEs make for an ideal system for the study of transcriptional dynamics. For example, LITEs may be used to study the dynamics of mRNA splice variant production upon induced expression of a target gene. On the other end of the transcription cycle, mRNA degradation studies are often performed in response to a strong extracellular stimulus, causing expression level changes in a plethora of genes. LITEs may be utilized to reversibly induce transcription of an endogenous target, after which point stimulation may be stopped and the degradation kinetics of the unique target may be tracked.

The temporal precision of LITEs may provide the power to time genetic regulation in concert with experimental interventions. For example, targets with suspected involvement in long-term potentiation (LTP) may be modulated in organotypic or dissociated neuronal cultures, but only during stimulus to induce LTP, so as to avoid interfering with the normal development of the cells. Similarly, in cellular models exhibiting disease phenotypes, targets suspected to be involved in the effectiveness of a particular therapy may be modulated only during treatment. Conversely, genetic targets may be modulated only during a pathological stimulus. Any number of experiments in which timing of genetic cues to external experimental stimuli is of relevance may potentially benefit from the utility of LITE modulation.

The in vivo context offers equally rich opportunities for the use of LITEs to control gene expression. As mentioned above, photoinducibility provides the potential for previously unachievable spatial precision. Taking advantage of the development of optrode technology, a stimulating fiber optic lead may be placed in a precise brain region. Stimulation region size may then be tuned by light intensity. This may be done in conjunction with the delivery of LITEs via viral vectors, or, if transgenic LITE animals were to be made available, may eliminate the use of viruses while still allowing for the modulation of gene expression in precise brain regions. LITEs may be used in a transparent organism, such as an immobilized zebrafish, to allow for extremely precise laser induced local gene expression changes.

LITEs may also offer valuable temporal precision in vivo. LITEs may be used to alter gene expression during a particular stage of development, for example, by repressing a particular apoptosis gene only during a particular stage of C. elegans growth. LITEs may be used to time a genetic cue to a particular experimental window. For example, genes implicated in learning may be overexpressed or repressed only during the learning stimulus in a precise region of the intact rodent or primate brain. Further, LITEs may be used to induce gene expression changes only during particular stages of disease development. For example, an oncogene may be overexpressed only once a tumor reaches a particular size or metastatic stage. Conversely, proteins suspected in the development of Alzheimer's may be knocked down only at defined time points in the animal's life and within a particular brain region. Although these examples do not exhaustively list the potential applications of the LITE system, they highlight some of the areas in which LITEs may be a powerful technology.

### Example 3

### Development of Mammalian TALE Transcriptional Repressors

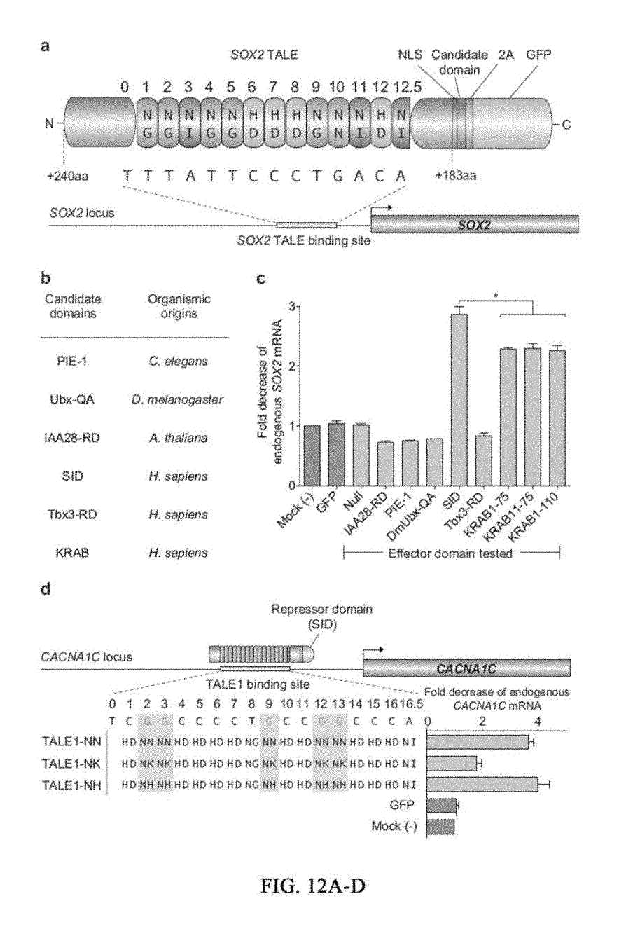

Applicants developed mammalian TALE repressor architectures to enable researchers to suppress transcription of endogenous genes. TALE repressors have the potential to suppress the expression of genes as well as non-coding transcripts such as microRNAs, rendering them a highly desirable tool for testing the causal role of specific genetic elements. In order to identify a suitable repression domain for use with TALEs in mammalian cells, a TALE targeting the promoter of the human SOX2 gene was used to evaluate the transcriptional repression activity of a collection of candidate repression domains (FIG. 12a). Repression domains across a range of eukaryotic host species were selected to increase the chance of finding a potent synthetic repressor, including the PIE-1 repression domain (PIE-i) (Batchelder, C. et al. Transcriptional repression by the Caenorhabditis elegans germ-line protein PIE-1. Genes Dev. 13, 202-212 (1999)) from Caenorhabditis elegans, the QA domain within the Ubx gene (Ubx-QA) (Tour, E., Hittinger, C. T. & McGinnis, W. Evolutionarily conserved domains required for activation and repression functions of the Drosophila Hox protein Ultrabithorax. Development 132, 5271-5281 (2005)) from Drosophila melanogaster, the IAA28 repression domain (IAA28-RD)(4) from Arabidopsis thaliana, the mSin interaction domain (SID) (Ayer, D. E., Laherty, C. D., Lawrence, Q. A., Armstrong, A. P. & Eisenman, R. N. Mad proteins contain a dominant transcription repression domain. Mol. Cell. Biol. 16, 5772-5781 (1996)), Tbx3 repression domain (Tbx3-RD), and the Krüppel-associated box (KRAB) (Margolin, J. F. et al. Kruppel-associated boxes are potent transcriptional repression domains. Proc. Natl. Acad. Sci. USA 91, 4509-4513 (1994)) repression domain from Homo Sapiens. Since different truncations of KRAB have been known to exhibit varying levels of transcriptional repression (Margolin, J. F. et al. Kruppel-associated boxes are potent transcriptional repression domains. Proc. Natl. Acad. Sci. USA 91, 4509-4513 (1994)), three different truncations of KRAB were tested (FIG. 12c). These candidate TALE repressors were expressed in HEK 293FTcells and it was found that TALEs carrying two widely used mammalian transcriptional repression domains, the SID (Ayer, D. E., Laherty, C. D., Lawrence, Q. A., Armstrong, A. P. & Eisenman, R.N. Mad proteins contain a dominant transcription repression domain. Mol. Cell. Biol. 16, 5772-5781 (1996)) and KRAB (Margolin, J. F. et al. Kruppel-associated boxes are potent transcriptional repression domains. Proc. Natl. Acad. Sci. USA 91, 4509-4513 (1994)) domains, were able to repress endogenous SOX2 expression, while the other domains had little effect on transcriptional activity (FIG. 12c). To control for potential perturbation of SOX2 transcription due to TALE binding, expression of the SOX2-targeting TALE DNA binding domain alone without any effector domain had no effect (similar to mock or expression of GFP) on the transcriptional activity of SOX2 (FIG. 12c, Null condition). Since the SID domain was able to achieve 26% more transcriptional repression of the endogenous SOX2 locus than the KRAB domain (FIG. 12c), it was decided to use the SID domain for subsequent studies.

To further test the effectiveness of the SID repressor domain for down regulating endogenous transcription, SID was combined with CACNA1C-target TALEs from the previous experiment (FIG. 12d). Using qRT-PCR, it was found that replacement of the VP64 domain on CACNA1C-targeting TALEs with SID was able to repress CACNA1C transcription. The NH-containing TALE repressor was able to achieve a similar level of transcriptional repression as the NN-containing TALE (˜4 fold repression), while the TALE repressor using NK was significantly less active (˜2 fold repression) (FIG. 12d). These data demonstrate that SID is indeed a suitable repression domain, while also further supporting NH as a more suitable G-targeting RVD than NK.

TALEs may be easily customized to recognize specific sequences on the endogenous genome. Here, a series of screens were conducted to address two important limitations of the TALE toolbox. Together, the identification of a more stringent G-specific RVD with uncompromised activity strength as well as a robust TALE repressor architecture further expands the utility of TALEs for probing mammalian transcription and genome function.

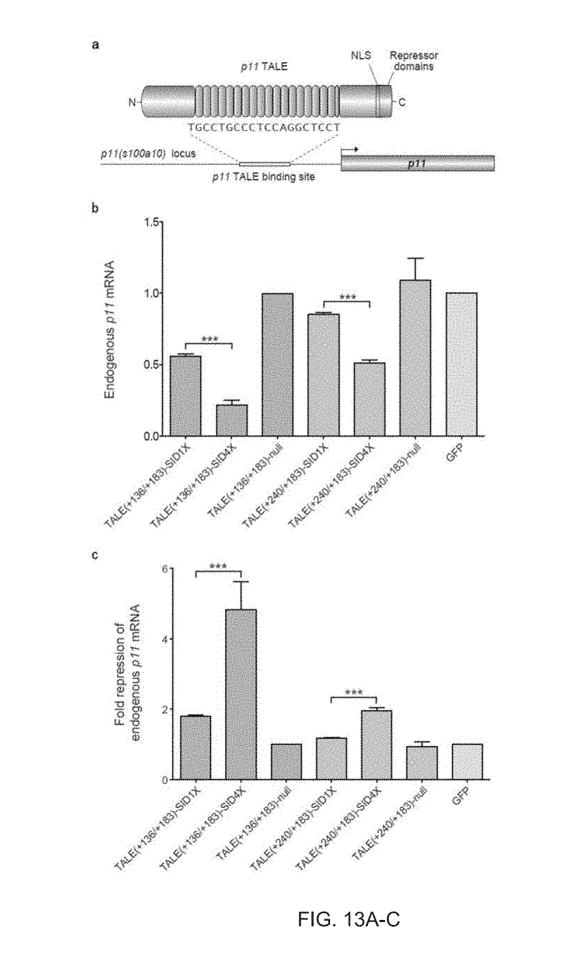

After identifying SID (mSin interaction domain) as a robust novel repressor domain to be used with TALEs, more active repression domain architecture based on SID domain for use with TALEs in mammalian cells were further designed and verified. This domain is called SID4X, which is a tandem repeat of four SID domains linked by short peptide linkers. For testing different TALE repressor architectures, a TALE targeting the promoter of the mouse (Mus musculus) p11 (s100a10) gene was used to evaluate the transcriptional repression activity of a series of candidate TALE repressor architectures (FIG. 13a). Since different truncations of TALE are known to exhibit varying levels of transcriptional activation activity, two different truncations of TALE fused to SID or SID4X domain were tested, one version with 136 and 183 amino acids at N- and C-termini flanking the DNA binding tandem repeats, with another one retaining 240 and 183 amino acids at N- and C-termini (FIG. 13b, c). The candidate TALE repressors were expressed in mouse Neuro2A cells and it was found that TALEs carrying both SID and SID4X domains were able to repress endogenous p11 expression up to 4.8 folds, while the GFP-encoding negative control construct had no effect on transcriptional of target gene (FIG. 13b, c). To control for potential perturbation of p11 transcription due to TALE binding, expression of the p11-targeting TALE DNA binding domain (with the same N- and C-termini truncations as the tested constructs) without any effector domain had no effect on the transcriptional activity of endogenous p11 (FIG. 13b, c, null constructs).

Because the constructs harboring SID4X domain were able to achieve 167% and 66% more transcriptional repression of the endogenous p11 locus than the SID domain depending on the truncations of TALE DNA binding domain (FIG. 13c), it was concluded that a truncated TALE DNA binding domain, bearing 136 and 183 amino acids at N- and C-termini respectively, fused to the SID4X domain is a potent TALE repressor architecture that enables down-regulation of target gene expression and is more active than the previous design employing SID domain.

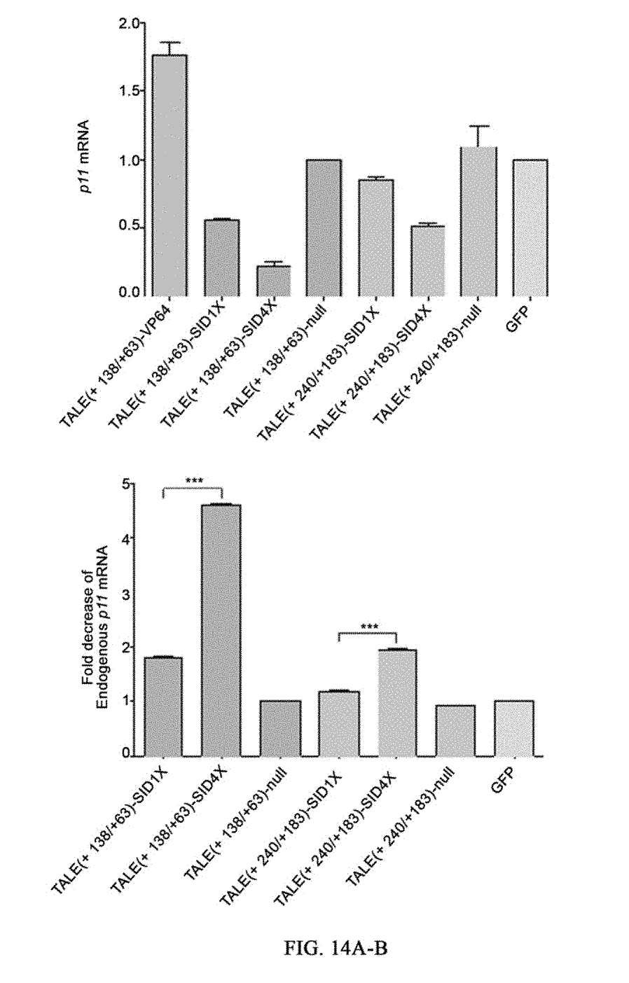

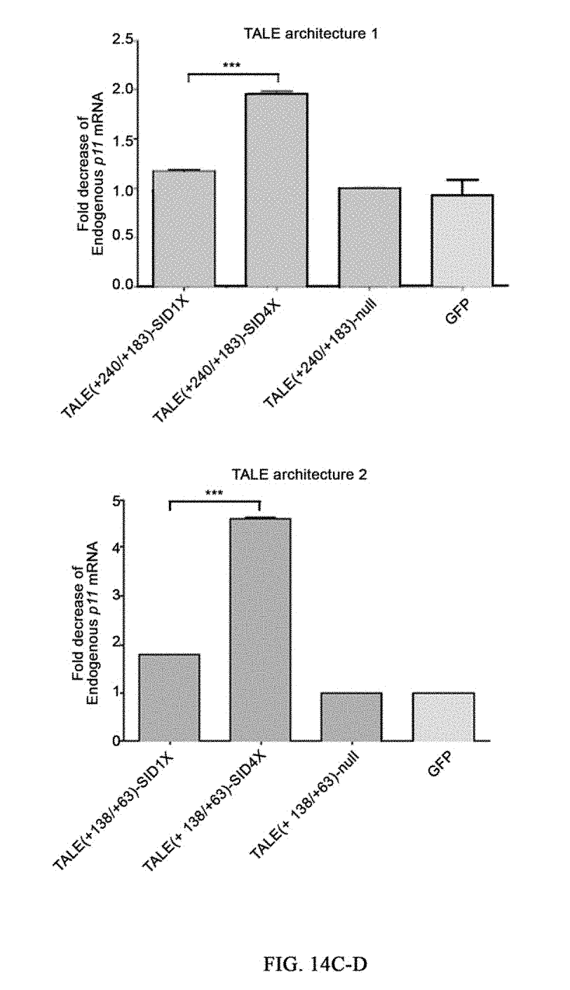

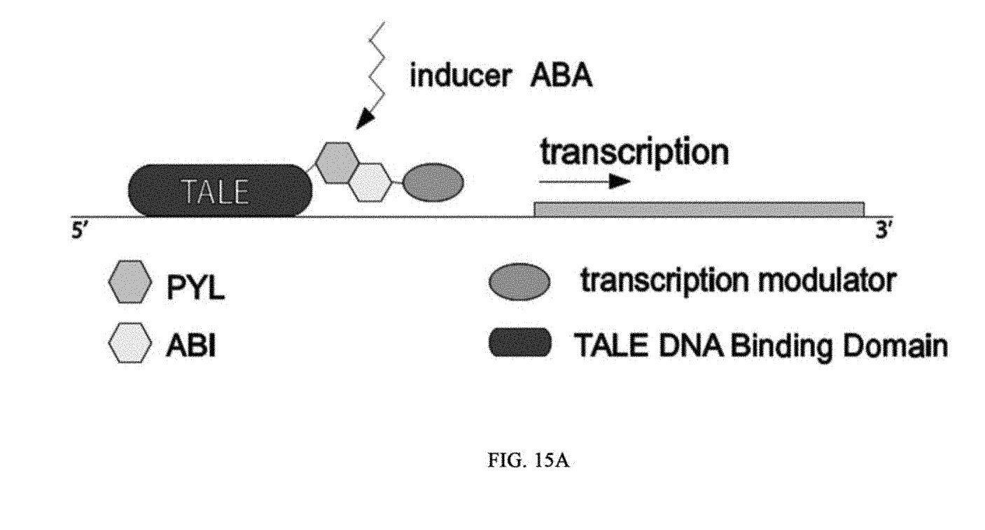

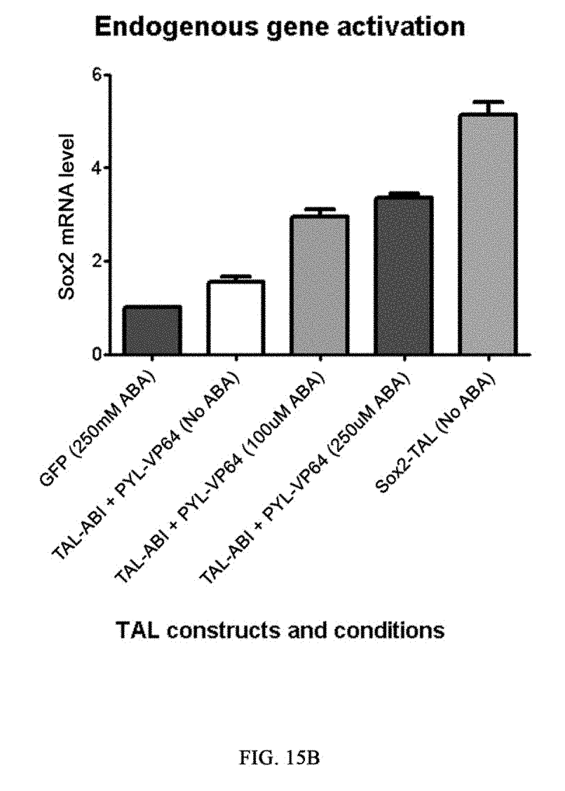

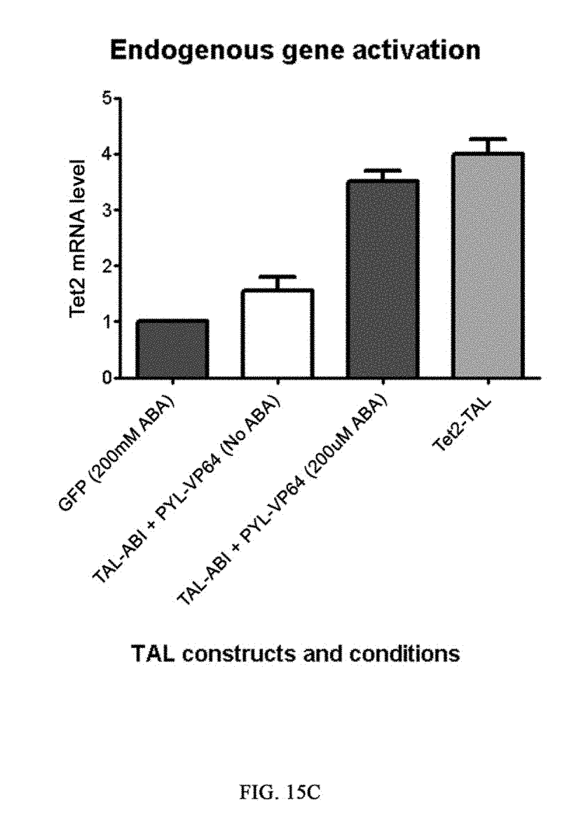

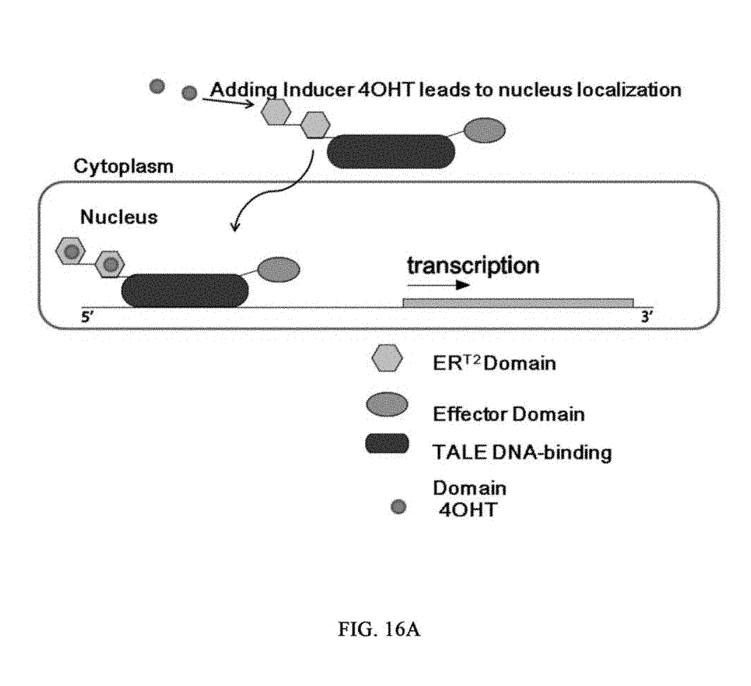

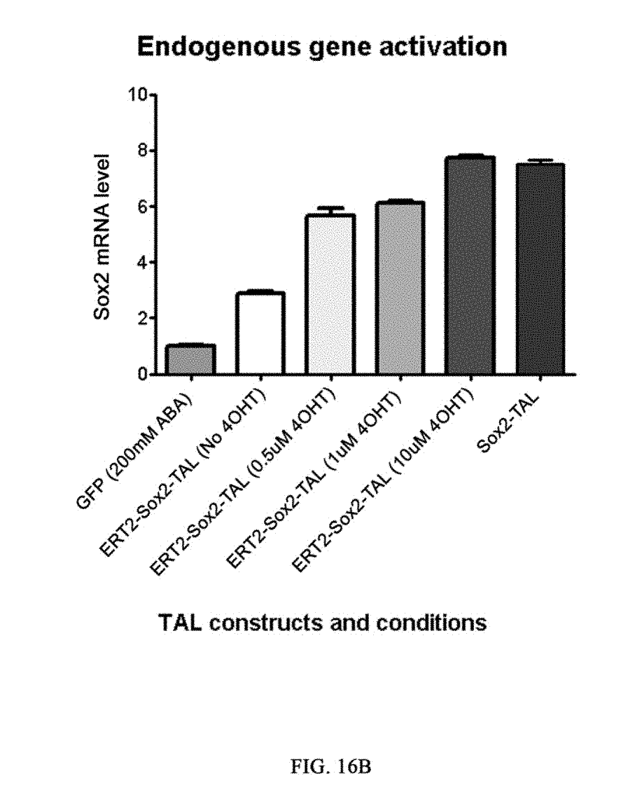

The mSin interaction domain (SID) and SID4X domain were codon optimized for mammalian expression and synthesized with flanking NheI and XbaI restriction sites (Genscript). Truncation variants of the TALE DNA binding domains are PCR amplified and fused to the SID or the SID4X domain using NheI and XbaI restriction sites. To control for any effect on transcription resulting from TALE binding, expression vectors carrying the TALE DNA binding domain alone using PCR cloning were constructed. The coding regions of all constructs were completely verified using Sanger sequencing. A comparison of two different types of TALE architecture is seen in FIG. 14.

### Example 4

### Development of Mammalian TALE Transcriptional Activators and Nucleases

Customized TALEs may be used for a wide variety of genome engineering applications, including transcriptional modulation and genome editing. Here, Applicants describe a toolbox for rapid construction of custom TALE transcription factors (TALE-TFs) and nucleases (TALENs) using a hierarchical ligation procedure. This toolbox facilitates affordable and rapid construction of custom TALE-TFs and TALENs within 1 week and may be easily scaled up to construct TALEs for multiple targets in parallel. Applicants also provide details for testing the activity in mammalian cells of custom TALE-TFs and TALENs using quantitative reverse-transcription PCR and Surveyor nuclease, respectively. The TALE toolbox will enable a broad range of biological applications.

TALEs are natural bacterial effector proteins used by Xanthomonas sp. to modulate gene transcription in host plants to facilitate bacterial colonization (Boch, J. & Bonas, U. Xanthomonas AvrBs3 family-type III effectors: discovery and function. Annu. Rev. Phytopathol. 48, 419-436 (2010) and Bogdanove, A. J., Schornack, S. & Lahaye, T. TAL effectors: finding plant genes for disease and defense. Curr. Opin. Plant Biol. 13, 394-401 (2010)). The central region of the protein contains tandem repeats of 34-aa sequences (termed monomers) that are required for DNA recognition and binding (Romer, P. et al. Plant pathogen recognition mediated by promoter activation of the pepper Bs3 resistance gene. Science 318, 645-648 (2007); Kay, S., Hahn, S., Marois, E., Hause, G. & Bonas, U. A bacterial effector acts as a plant transcription factor and induces a cell size regulator. Science 318, 648-651 (2007); Kay, S., Hahn, S., Marois, E., Wieduwild, R. & Bonas, U. Detailed analysis of the DNA recognition motifs of the Xanthomonas type III effectors AvrBs3 and AvrBs3Deltarep16. Plant J. 59, 859-871 (2009) and Romer, P. et al. Recognition of AvrBs3-like proteins is mediated by specific binding to promoters of matching pepper Bs3 alleles. Plant Physiol. 150, 1697-1712 (2009).) (FIG. 8). Naturally occurring TALEs have been found to have a variable number of monomers, ranging from 1.5 to 33.5 (Boch, J. & Bonas, U. Xanthomonas AvrBs3 family-type III effectors: discovery and function. Annu. Rev. Phytopathol. 48, 419-436 (2010)). Although the sequence of each monomer is highly conserved, they differ primarily in two positions termed the repeat variable diresidues (RVDs, 12th and 13th positions). Recent reports have found that the identity of these two residues determines the nucleotide-binding specificity of each TALE repeat and that a simple cipher specifies the target base of each RVD (NI=A, HD=C, NG=T, NN=G or A) (Boch, J. et al. Breaking the code of DNA binding specificity of TAL-type III effectors. Science 326, 1509-1512 (2009) and Moscou, M. J. & Bogdanove, A. J. A simple cipher governs DNA recognition by TAL effectors. Science 326, 1501 (2009)). Thus, each monomer targets one nucleotide and the linear sequence of monomers in a TALE specifies the target DNA sequence in the 5′ to 3′ orientation. The natural TALE-binding sites within plant genomes always begin with a thymine (Boch, J. et al. Breaking the code of DNA binding specificity of TAL-type III effectors. Science 326, 1509-1512 (2009) and Moscou, M. J. & Bogdanove, A. J. A simple cipher governs DNA recognition by TAL effectors. Science 326, 1501 (2009)), which is presumably specified by a cryptic signal within the nonrepetitive N terminus of TALEs. The tandem repeat DNA-binding domain always ends with a half-length repeat (0.5 repeat, FIG. 8). Therefore, the length of the DNA sequence being targeted is equal to the number of full repeat monomers plus two.

In plants, pathogens are often host-specific. For example, Fusarium oxysporum f. sp. lycopersici causes tomato wilt but attacks only tomato, and F. oxysporum f. dianthii Puccinia graminis f. sp. tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield, Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to induce mutations. Accordingly, one skilled in the art can analyze the genome of sources of resistance genes, and in Varieties having desired characteristics or traits employ the present invention to induce the rise of resistance genes, with more precision than previous mutagenic agents and hence accelerate and improve plant breeding programs.

Applicants have further improved the TALE assembly system with a few optimizations, including maximizing the dissimilarity of ligation adaptors to minimize misligations and combining separate digest and ligation steps into single Golden Gate (Engler, C., Kandzia, R. & Marillonnet, S. A one pot, one step, precision cloning method with high throughput capability. PLoS ONE 3, e3647 (2008); Engler, C., Gruetzner, R., Kandzia, R. & Marillonnet, S. Golden gate shuffling: a one-pot DNA shuffling method based on type IIs restriction enzymes. PLoS ONE 4, e5553 (2009) and Weber, E., Engler, C., Gruetzner, R., Werner, S. & Marillonnet, S. A modular cloning system for standardized assembly of multigene constructs. PLoS ONE 6, e16765 (2011)) reactions. Briefly, each nucleotide-specific monomer sequence is amplified with ligation adaptors that uniquely specify the monomer position within the TALE tandem repeats. Once this monomer library is produced, it may conveniently be reused for the assembly of many TALEs. For each TALE desired, the appropriate monomers are first ligated into hexamers, which are then amplified via PCR. Then, a second Golden Gate digestion-ligation with the appropriate TALE cloning backbone (FIG. 8) yields a fully assembled, sequence-specific TALE. The backbone contains a ccdB negative selection cassette flanked by the TALE N and C termini, which is replaced by the tandem repeat DNA-binding domain when the TALE has been successfully constructed. ccdB selects against cells transformed with an empty backbone, thereby yielding clones with tandem repeats inserted (Cermak, T. et al. Efficient design and assembly of custom TALEN and other TAL effector-based constructs for DNA targeting. Nucleic Acids Res. 39, e82 (2011)).

Assemblies of monomeric DNA-binding domains may be inserted into the appropriate TALE-TF or TALEN cloning backbones to construct customized TALE-TFs and TALENs. TALE-TFs are constructed by replacing the natural activation domain within the TALE C terminus with the synthetic transcription activation domain VP64 (Zhang, F. et al. Efficient construction of sequence-specific TAL effectors for modulating mammalian transcription. Nat. Biotechnol. 29, 149-153 (2011); FIG. 8). By targeting a binding site upstream of the transcription start site, TALE-TFs recruit the transcription complex in a site-specific manner and initiate gene transcription. TALENs are constructed by fusing a C-terminal truncation (+63 aa) of the TALE DNA-binding domain (Miller, J. C. et al. A TALE nuclease architecture for efficient genome editing. Nat. Biotechnol. 29, 143-148 (2011)) with the nonspecific FokI endonuclease catalytic domain (FIG. 14). The +63-aa C-terminal truncation has also been shown to function as the minimal C terminus sufficient for transcriptional modulation (Zhang, F. et al. Efficient construction of sequence-specific TAL effectors for modulating mammalian transcription. Nat. Biotechnol. 29, 149-153 (2011)). TALENs form dimers through binding to two target sequences separated by ˜17 bases. Between the pair of binding sites, the FokI catalytic domains dimerize and function as molecular scissors by introducing double-strand breaks (DSBs; FIG. 8). Normally, DSBs are repaired by the nonhomologous end-joining (Huertas, P. DNA resection in eukaryotes: deciding how to fix the break. Nat. Struct. Mol. Biol. 17, 11-16 (2010)) pathway (NHEJ), resulting in small deletions and functional gene knockout. Alternatively, TALEN-mediated DSBs may stimulate homologous recombination, enabling site-specific insertion of an exogenous donor DNA template (Miller, J. C. et al. A TALE nuclease architecture for efficient genome editing. Nat. Biotechnol. 29, 143-148 (2011) and Hockemeyer, D. et al. Genetic engineering of human pluripotent cells using TALE nucleases. Nat. Biotechnol. 29, 731-734 (2011)).

Along with the TALE-TFs being constructed with the VP64 activation domain, other embodiments of the invention relate to TALE polypeptides being constructed with the VP16 and p65 activation domains. A graphical comparison of the effect these different activation domains have on Sox2 mRNA level is provided in FIG. 11.

### Example 5

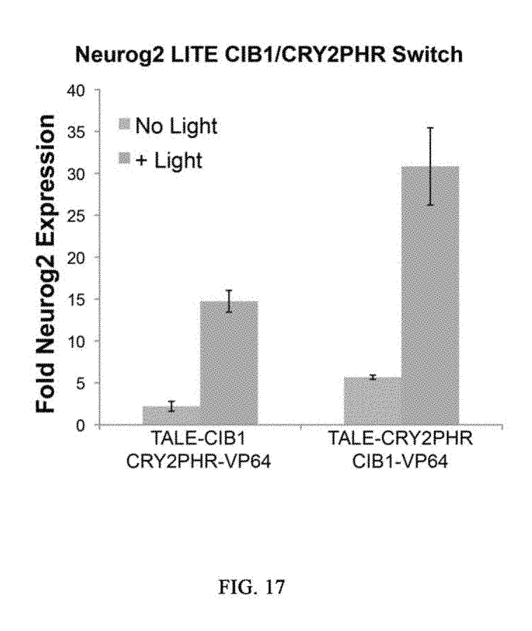

FIG. 17 depicts an effect of cryptochrome2 heterodimer orientation on LITE functionality. Two versions of the Neurogenin 2 (Neurog2) LITE were synthesized to investigate the effects of cryptochrome 2 photolyase homology region (CRY2PHR)/calcium and integrin-binding protein 1 (CIB1) dimer orientation. In one version, the CIB1 domain was fused to the C-terminus of the TALE (Neurog2) domain, while the CRY2PHR domain was fused to the N-terminus of the VP64 domain. In the converse version, the CRY2PHR domain was fused to the C-terminus of the TALE (Neurog2) domain, while the CIB1 domain was fused to the N-terminus of the VP64 domain. Each set of plasmids were transfected in Neuro2a cells and stimulated (466 nm, 5 mW/cm², 1 sec pulse per 15 sec, 12 h) before harvesting for qPCR analysis. Stimulated LITE and unstimulated LITE Neurog2 expression levels were normalized to Neurog2 levels from stimulated GFP control samples. The TALE-CRY2PHR/CIB1-VP64 LITE exhibited elevated basal activity and higher light induced Neurog2 expression, and suggested its suitability for situations in which higher absolute activation is required. Although the relative light inducible activity of the TALE-CIB1/CRY2PHR-VP64 LITE was lower that its counterpart, the lower basal activity suggested its utility in applications requiring minimal baseline activation. Further, the TALE-CIB1 construct was smaller in size, compared to the TALE-CRY2PHR construct, a potential advantage for applications such as viral packaging.

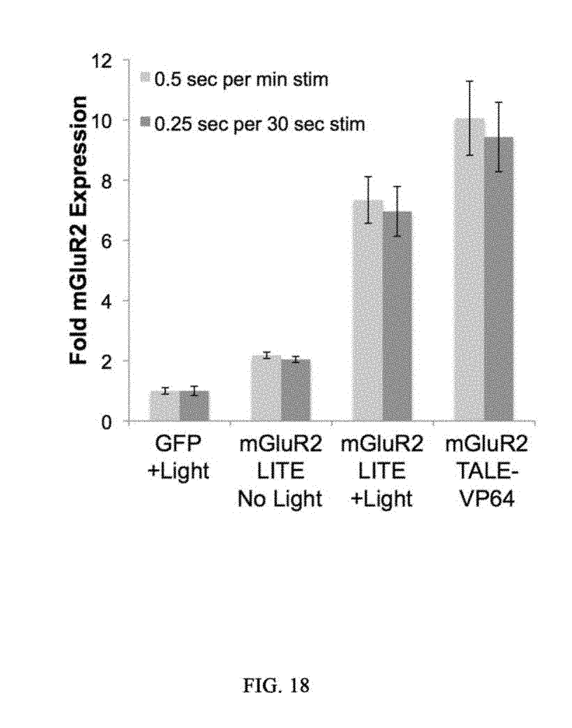

FIG. 18 depicts metabotropic glutamate receptor 2 (mGlur2) LITE activity in mouse cortical neuron culture. A mGluR2 targeting LITE was constructed via the plasmids pAAV-human Synapsin I promoter (hSyn)-HA-TALE(mGluR2)-CIB1 and pAAV-hSyn-CRY2PHR-VP64-2A-GFP. These fusion constructs were then packaged into adeno associated viral vectors (AAV). Additionally, AAV carrying hSyn-TALE-VP64-2A-GFP and GFP only were produced. Embryonic mouse (E16) cortical cultures were plated on Poly-L-lysine coated 24 well plates. After 5 days in vitro neural cultures were co-transduced with a mixture of TALE(mGluR2)-CIB1 and CRY2PHR-VP64 AAV stocks. Control samples were transduced with either TALE(mGluR2)-VP64 AAV or GFP AAV. 6 days after AAV transduction, experimental samples were stimulated using either of two light pulsing paradigms: 0.5 s per min and 0.25 sec per 30 sec. Neurons were stimulated for 24 h and harvested for qPCR analysis. All mGluR2 expression levels were normalized to the respective stimulated GFP control. The data suggested that the LITE system could be used to induce the light-dependent activation of a target gene in primary neuron cultures in vitro.

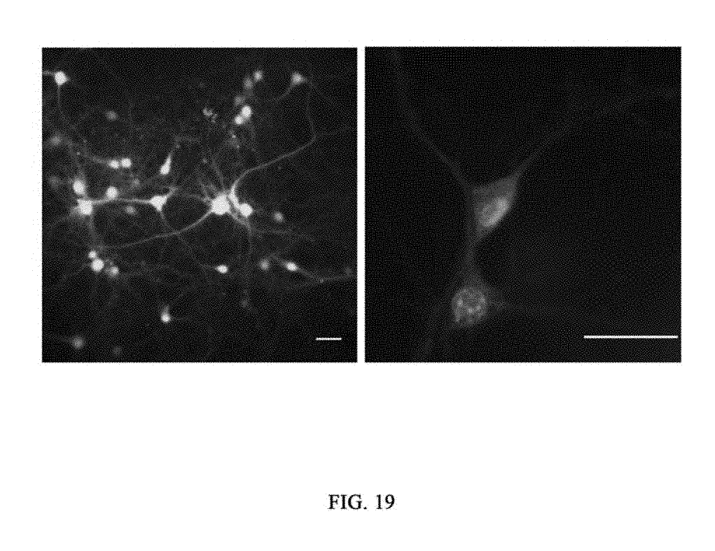

FIG. 19 depicts transduction of primary mouse neurons with LITE AAV vectors. Primary mouse cortical neuron cultures were co-transduced at 5 days in vitro with AAV vectors encoding hSyn-CRY2PHR-VP64-2A-GFP and hSyn-HA-TALE-CIB1, the two components of the LITE system. Left panel: at 6 days after transduction, neural cultures exhibited high expression of GFP from the hSyn-CRY2PHR-VP64-2A-GFP vector. Right panel: Co-transduced neuron cultures were fixed and stained with an antibody specific to the HA epitope on the N-terminus of the TALE domain in hSyn-HA-TALE-CIB1. Red signal indicated HA expression, with particularly strong nuclear signal (DNA stained by DAPI in blue channel). Together these images suggested that the expression of each LITE component could be achieved in primary mouse neuron cultures. (scale bars=50 um).

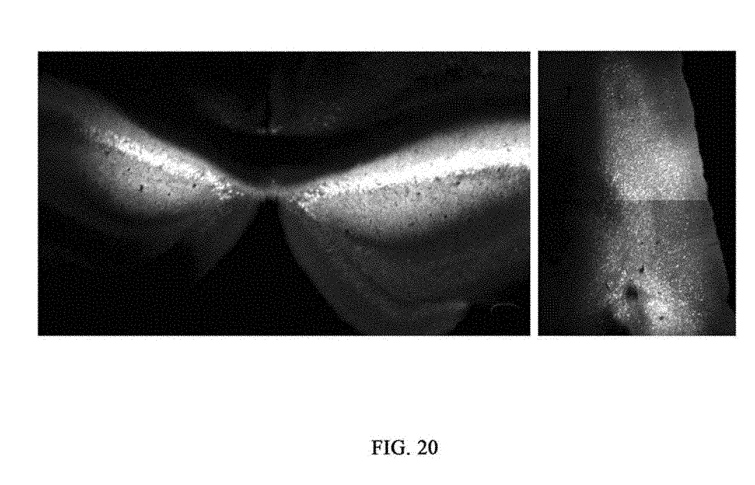

FIG. 20 depicts expression of a LITE component in vivo. An AAV vector of seratype 1/2 carrying hSyn-CRY2PHR-VP64 was produced via transfection of HEK293FT cells and purified via heparin column binding. The vector was concentrated for injection into the intact mouse brain. 1 uL of purified AAV stock was injected into the hippocampus and infralimbic cortex of an 8 week old male C57BL/6 mouse by steroeotaxic surgery and injection. 7 days after in vivo transduction, the mouse was euthanized and the brain tissue was fixed by paraformaldehyde perfusion. Slices of the brain were prepared on a vibratome and mounted for imaging. Strong and widespread GFP signals in the hippocampus and infralimbic cortex suggested efficient transduction and high expression of the LITE component CRY2PHR-VP64.

### Example 6

### Improved Design by Using NES Element

Estrogen receptor T2 (ERT2) has a leakage issue. The ERT2 domain would enter the nucleus even in the absence of 4-Hydroxytestosterone (4OHT), leading to a background level of activation of target gene by TAL. NES (nuclear exporting signal) is a peptide signal that targets a protein to the cytoplasm of a living cell. By adding NES to an existing construct, Applicants aim to prevent the entering of ERT2-TAL protein into nucleus in the absence of 4OHT, lowering the background activation level due to the “leakage” of the ERT2 domain.

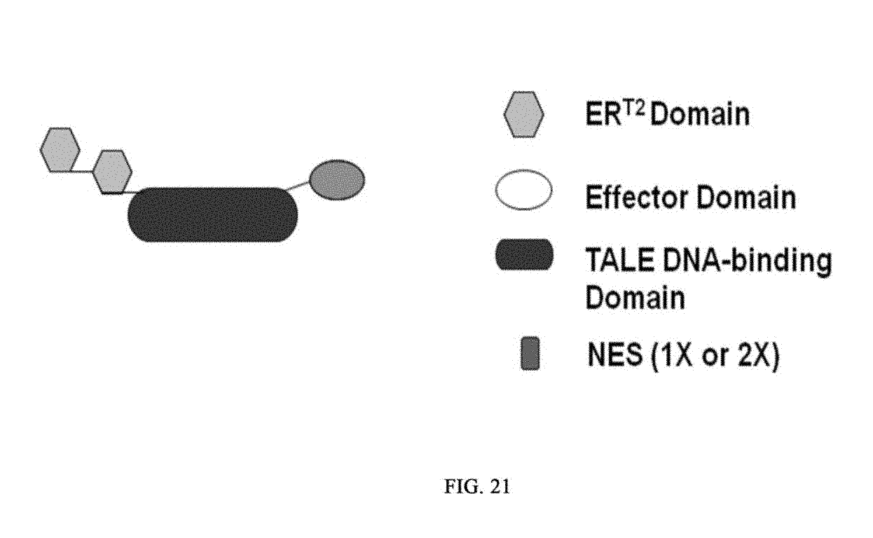

FIG. 21 depicts an improved design of the construct where the specific NES peptide sequence used is LDLASLIL (SEQ ID NO: 6).

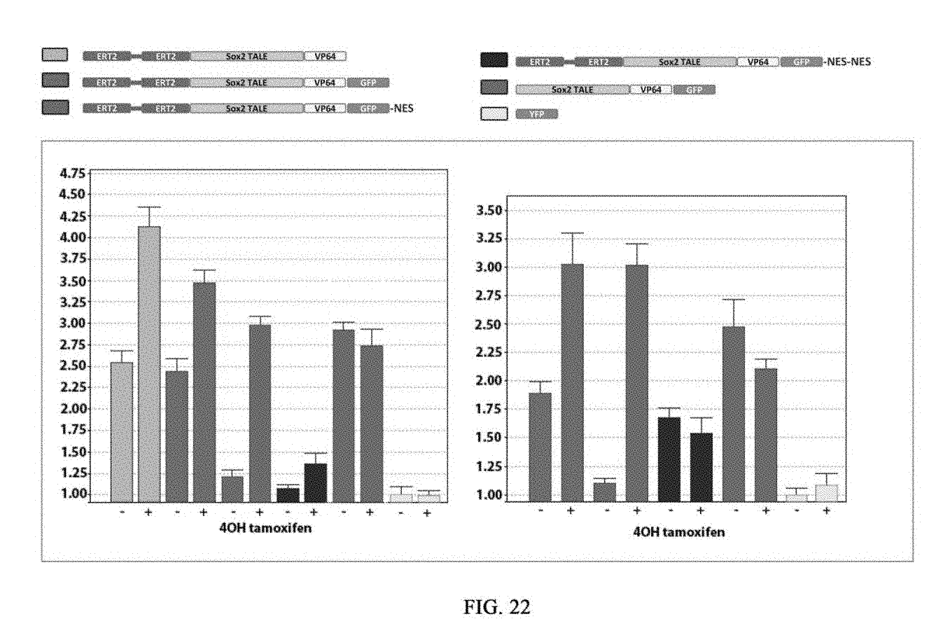

FIG. 22 depicts Sox2 mRNA levels in the absence and presence of 40H tamoxifen. Y-axis is Sox2 mRNA level as measured by qRT-PCR. X-axis is a panel of different construct designs described on top. Plus and minus signs indicate the presence or absence of 0.5 uM 4OHT.

### Example 7

### Multiplex Genome Engineering Using CRISPR/Cas Systems

Functional elucidation of causal genetic variants and elements requires precise genome editing technologies. The type II prokaryotic CRISPR (clustered regularly interspaced short palindromic repeats) adaptive immune system has been shown to facilitate RNA-guided site-specific DNA cleavage. Applicants engineered two different type II CRISPR systems and demonstrate that Cas9 nucleases can be directed by short RNAs to induce precise cleavage at endogenous genomic loci in human and mouse cells. Cas9 can also be converted into a nicking enzyme to facilitate homology-directed repair with minimal mutagenic activity. Finally, multiple guide sequences can be encoded into a single CRISPR array to enable simultaneous editing of several sites within the mammalian genome, demonstrating easy programmability and wide applicability of the CRISPR technology.

Prokaryotic CRISPR adaptive immune systems can be reconstituted and engineered to mediate multiplex genome editing in mammalian cells.

Precise and efficient genome targeting technologies are needed to enable systematic reverse engineering of causal genetic variations by allowing selective perturbation of individual genetic elements. Although genome-editing technologies such as designer zinc fingers (ZFs) (M. H. Porteus, D. Baltimore, Chimeric nucleases stimulate gene targeting in human cells. Science 300, 763 (May 2, 2003); J. C. Miller et al., An improved zinc-finger nuclease architecture for highly specific genome editing. Nat Biotechnol 25, 778 (July, 2007); J. D. Sander et al., Selection-free zinc-finger-nuclease engineering by context-dependent assembly (CoDA). Nat Methods 8, 67 (January, 2011) and A. J. Wood et al., Targeted genome editing across species using ZFNs and TALENs. Science 333, 307 (Jul. 15, 2011)), transcription activator-like effectors (TALEs) (A. J. Wood et al., Targeted genome editing across species using ZFNs and TALENs. Science 333, 307 (Jul. 15, 2011); M. Christian et al., Targeting DNA double-strand breaks with TAL effector nucleases. Genetics 186, 757 (October, 2010); F. Zhang et al., Efficient construction of sequence-specific TAL effectors for modulating mammalian transcription. Nat Biotechnol 29, 149 (February, 2011); J. C. Miller et al., A TALE nuclease architecture for efficient genome editing. Nat Biotechnol 29, 143 (February, 2011); D. Reyon et al., FLASH assembly of TALENs for high-throughput genome editing. Nat Biotechnol 30, 460 (May, 2012); J. Boch et al., Breaking the code of DNA binding specificity of TAL-type III effectors. Science 326, 1509 (Dec. 11, 2009) and M. J. Moscou, A. J. Bogdanove, A simple cipher governs DNA recognition by TAL effectors. Science 326, 1501 (Dec. 11, 2009)), and homing meganucleases (B. L. Stoddard, Homing endonuclease structure and function. Quarterly reviews of biophysics 38, 49 (February, 2005)) have begun to enable targeted genome modifications, there remains a need for new technologies that are scalable, affordable, and easy to engineer. Here, Applicants report the development of a new class of precision genome engineering tools based on the RNA-guided Cas9 nuclease (M. Jinek et al., A programmable dual-RNA-guided DNA endonuclease in adaptive bacterial immunity. Science 337, 816 (Aug. 17, 2012); G. Gasiunas, R. Barrangou, P. Horvath, V. Siksnys, Cas9-crRNA ribonucleoprotein complex mediates specific DNA cleavage for adaptive immunity in bacteria. Proc Natl Acad Sci USA 109, E2579 (Sep. 25, 2012) and J. E. Garneau et al., The CRISPR/Cas bacterial immune system cleaves bacteriophage and plasmid DNA. Nature 468, 67 (Nov. 4, 2010)) from the type II prokaryotic CRISPR adaptive immune system (H. Deveau, J. E. Garneau, S. Moineau, CRISPR/Cas system and its role in phage-bacteria interactions. Annual review of microbiology 64, 475 (2010); P. Horvath, R. Barrangou, CRISPR/Cas, the immune system of bacteria and archaea. Science 327, 167 (Jan. 8, 2010); K. S. Makarova et al., Evolution and classification of the CRISPR-Cas systems. Nat Rev Microbiol 9, 467 (June, 2011) and D. Bhaya, M. Davison, R. Barrangou, CRISPR-Cas systems in bacteria and archaea: versatile small RNAs for adaptive defense and regulation. Annu Rev Genet 45, 273 (2011)).

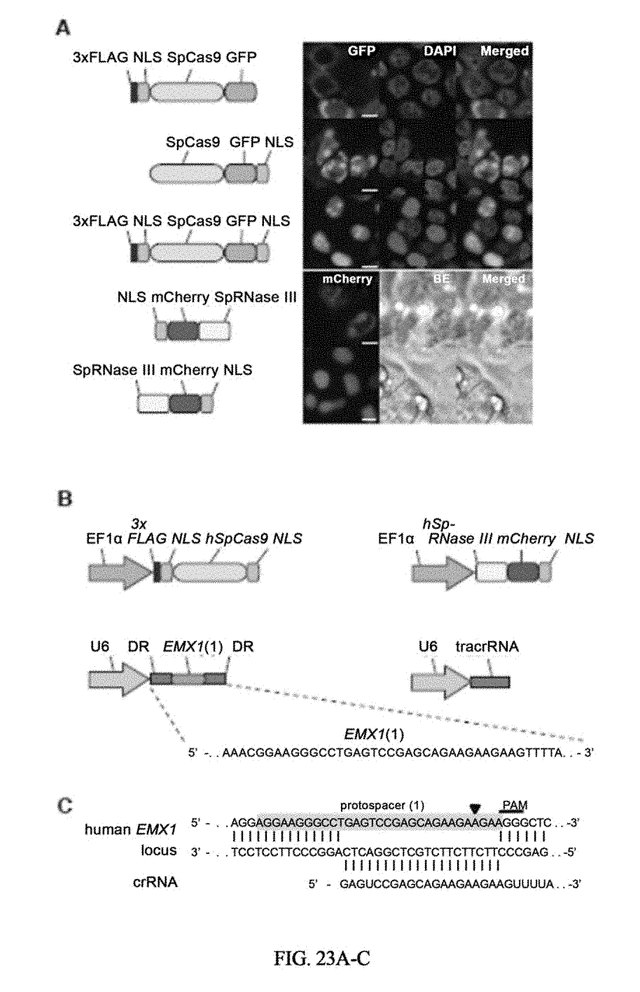

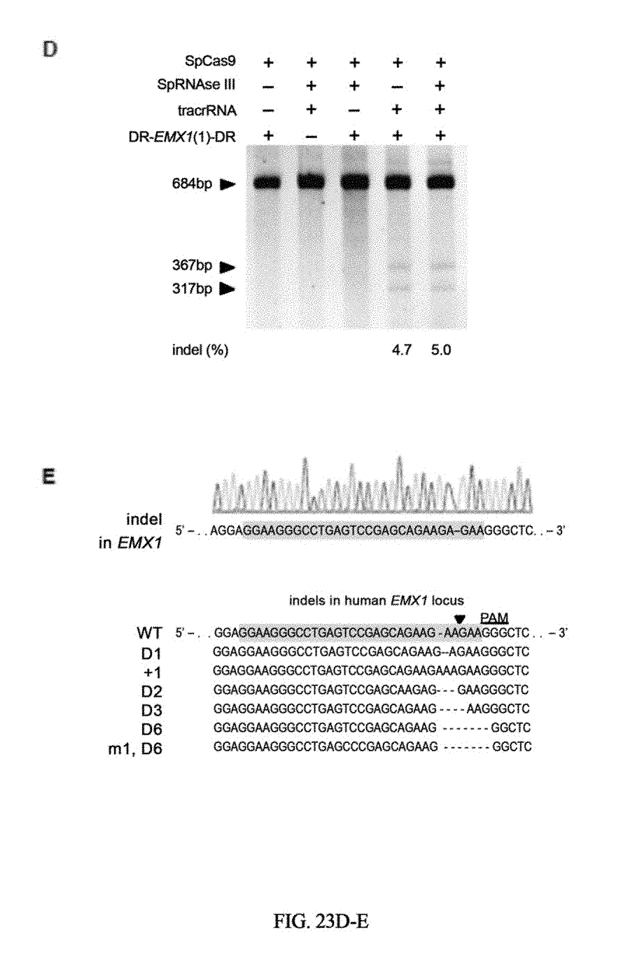

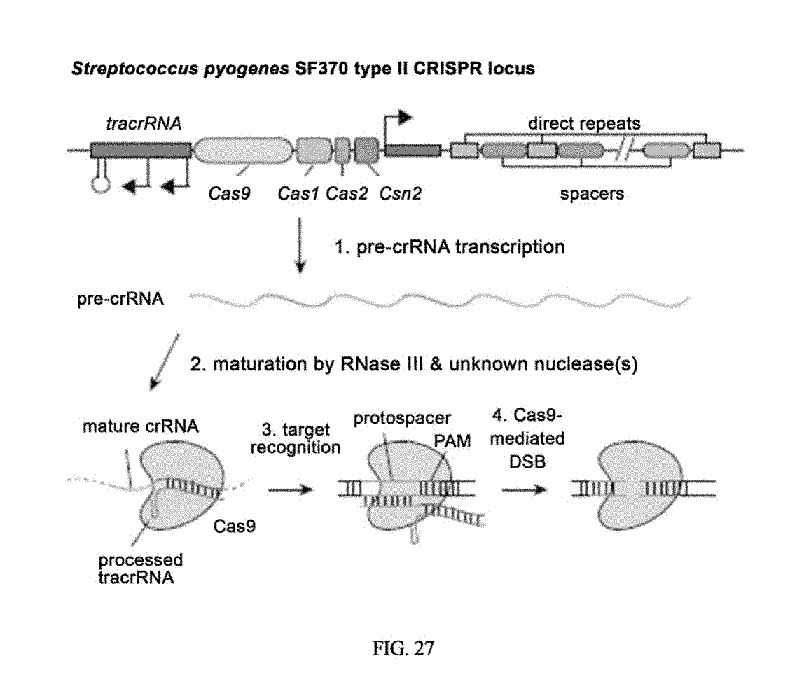

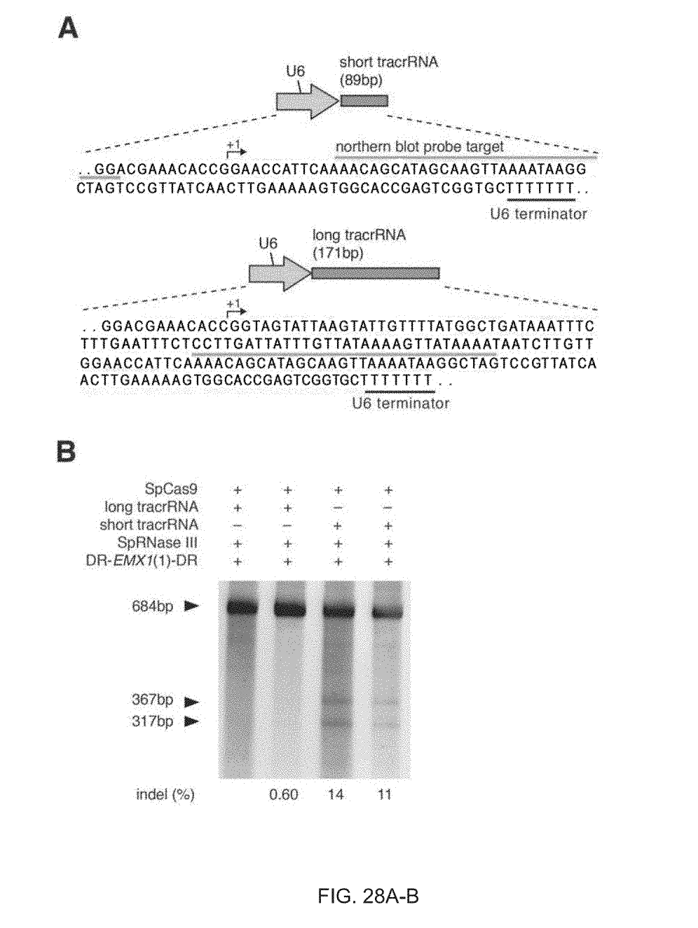

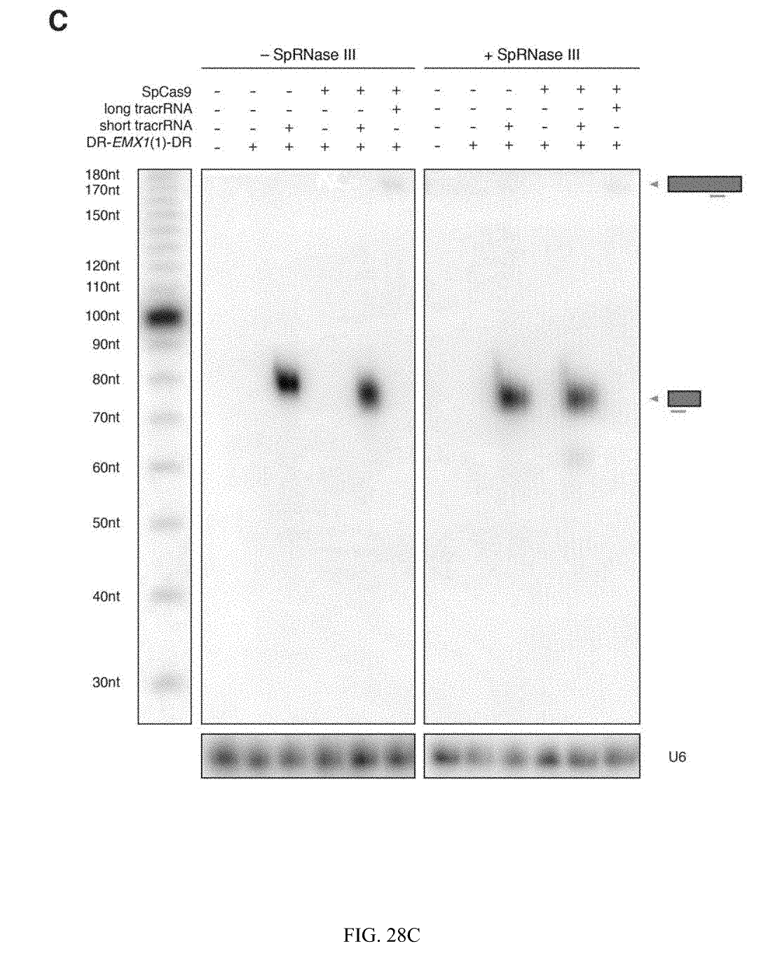

The Streptococcus pyogenes SF370 type II CRISPR locus consists of four genes, including the Cas9 nuclease, as well as two non-coding RNAs: tracrRNA and a pre-crRNA array containing nuclease guide sequences (spacers) interspaced by identical direct repeats (DRs) (FIG. 27) (E. Deltcheva et al., CRISPR RNA maturation by trans-encoded small RNA and host factor RNase III. Nature 471, 602 (Mar. 31, 2011)). Applicants sought to harness this prokaryotic RNA-programmable nuclease system to introduce targeted double stranded breaks (DSBs) in mammalian chromosomes through heterologous expression of the key components. It has been previously shown that expression of tracrRNA, pre-crRNA, host factor RNase III, and Cas9 nuclease are necessary and sufficient for cleavage of DNA in vitro (M. Jinek et al., A programmable dual-RNA-guided DNA endonuclease in adaptive bacterial immunity. Science 337, 816 (Aug. 17, 2012) and G. Gasiunas, R. Barrangou, P. Horvath, V. Siksnys, Cas9-crRNA ribonucleoprotein complex mediates specific DNA cleavage for adaptive immunity in bacteria. Proc Natl Acad Sci USA 109, E2579 (Sep. 25, 2012)) and in prokaryotic cells (R. Sapranauskas et al., The Streptococcus thermophilus CRISPR/Cas system provides immunity in Escherichia coli. Nucleic Acids Res 39, 9275 (November, 2011) and A. H. Magadan, M. E. Dupuis, M. Villion, S. Moineau, Cleavage of phage DNA by the Streptococcus thermophilus CRISPR3-Cas system. PLoS One 7, e40913 (2012)). Applicants codon optimized the S. pyogenes Cas9 (SpCas9) and RNase III (SpRNase III) and attached nuclear localization signals (NLS) to ensure nuclear compartmentalization in mammalian cells. Expression of these constructs in human 293FT cells revealed that two NLSs are required for targeting SpCas9 to the nucleus (FIG. 23A). To reconstitute the non-coding RNA components of CRISPR, Applicants expressed an 89-nucleotide (nt) tracrRNA (FIG. 28) under the RNA polymerase III U6 promoter (FIG. 23B). Similarly, Applicants used the U6 promoter to drive the expression of a pre-crRNA array comprising a single guide spacer flanked by DRs (FIG. 23B). Applicants designed an initial spacer to target a 30-basepair (bp) site (protospacer) in the human EMX1 locus that precedes an NGG, the requisite protospacer adjacent motif (PAM) (FIG. 23C and FIG. 27) (H. Deveau et al., Phage response to CRISPR-encoded resistance in Streptococcus thermophilus. J Bacteriol 190, 1390 (February, 2008) and F. J. Mojica, C. Diez-Villasenor, J. Garcia-Martinez, C. Almendros, Short motif sequences determine the targets of the prokaryotic CRISPR defence system. Microbiology 155, 733 (March, 2009)).

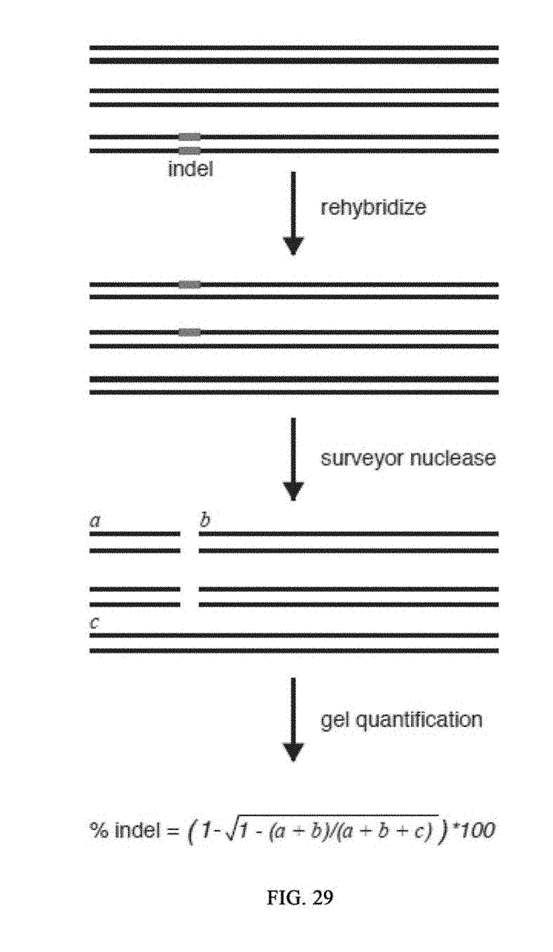

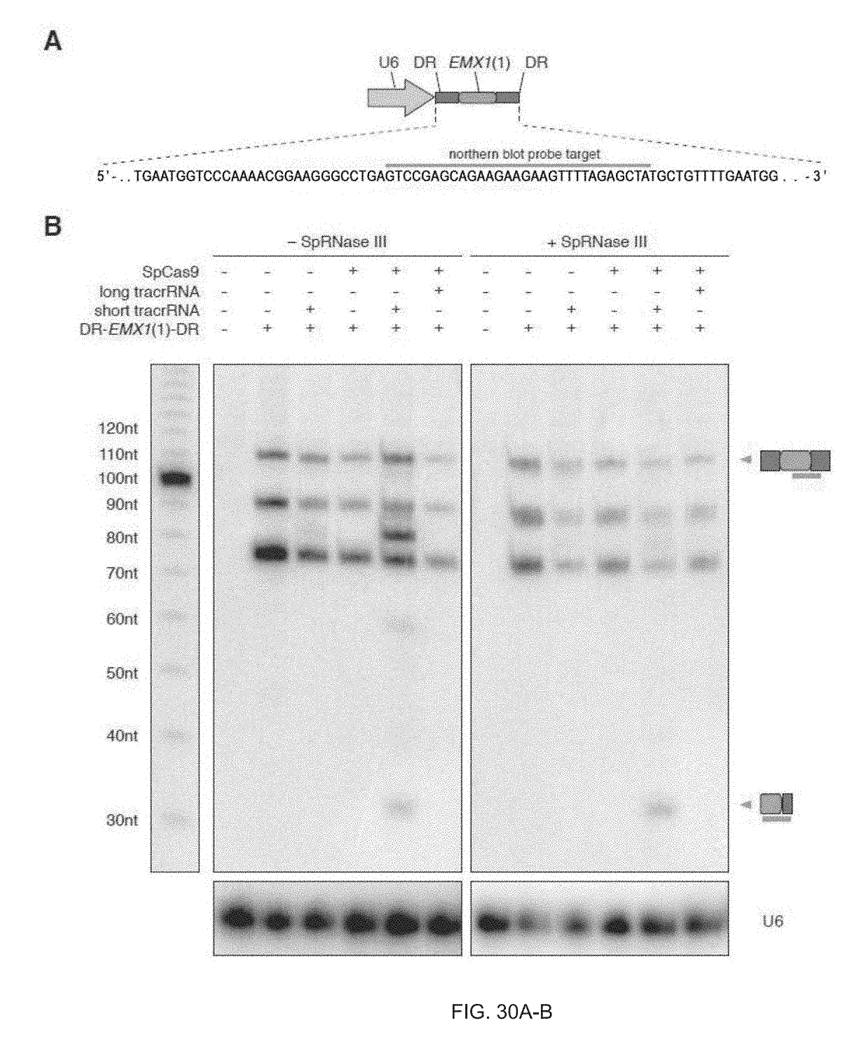

To test whether heterologous expression of the CRISPR system (SpCas9, SpRNase III, tracrRNA, and pre-crRNA) can achieve targeted cleavage of mammalian chromosomes, Applicants transfected 293FT cells with different combinations of CRISPR components. Since DSBs in mammalian DNA are partially repaired by the indel-forming non-homologous end joining (NHEJ) pathway, Applicants used the SURVEYOR assay (FIG. 29) to detect endogenous target cleavage (FIG. 23D and FIG. 28B). Co-transfection of all four required CRISPR components resulted in efficient cleavage of the protospacer (FIG. 23D and FIG. 28B), which is subsequently verified by Sanger sequencing (FIG. 23E). Interestingly, SpRNase III was not necessary for cleavage of the protospacer (FIG. 23D), and the 89-nt tracrRNA is processed in its absence (FIG. 28C). Similarly, maturation of pre-crRNA does not require RNase III (FIG. 23D and FIG. 30), suggesting that there may be endogenous mammalian RNases that assist in pre-crRNA maturation (M. Jinek, J. A. Doudna, A three-dimensional view of the molecular machinery of RNA interference. Nature 457, 405 (Jan. 22, 2009); C. D. Malone, G. J. Hannon, Small RNAs as guardians of the genome. Cell 136, 656 (Feb. 20, 2009) and G. Meister, T. Tuschl, Mechanisms of gene silencing by double-stranded RNA. Nature 431, 343 (Sep. 16, 2004)). Removing any of the remaining RNA or Cas9 components abolished the genome cleavage activity of the CRISPR system (FIG. 23D). These results define a minimal three-component system for efficient CRISPR-mediated genome modification in mammalian cells.

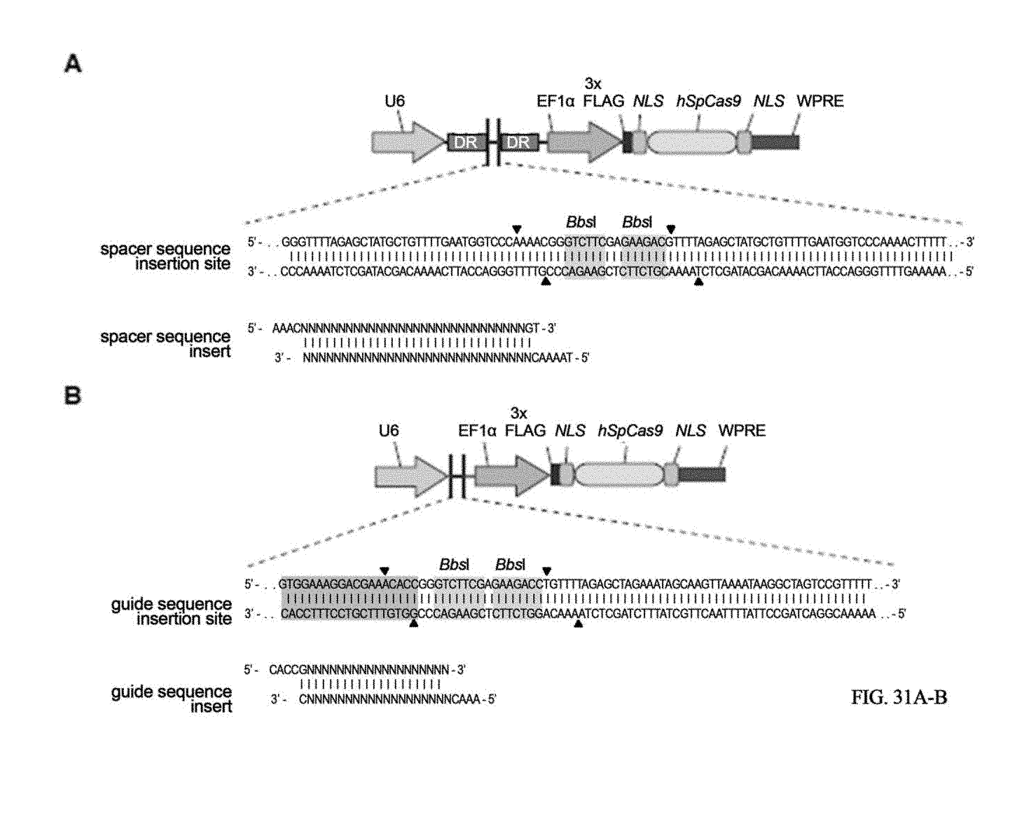

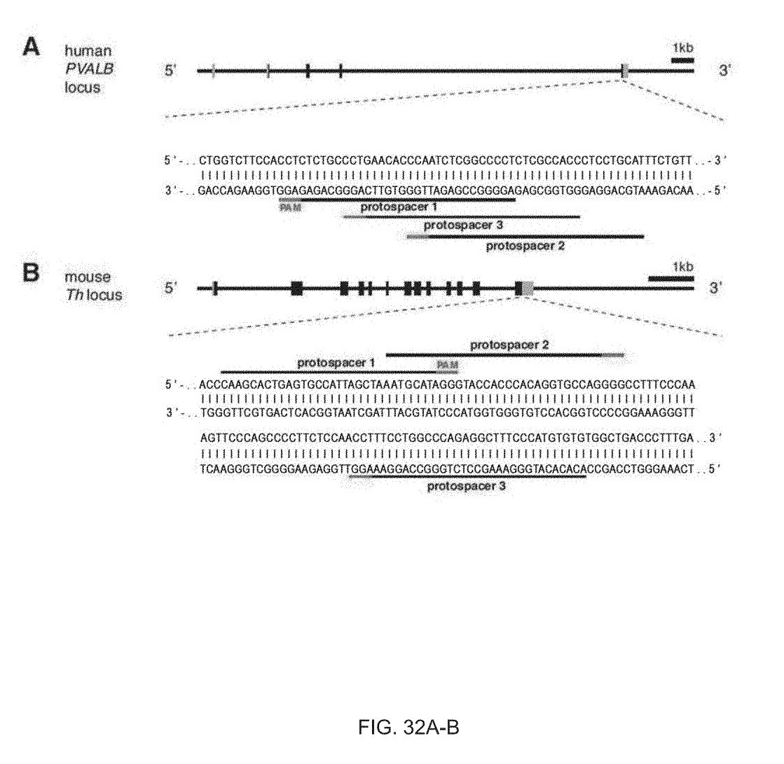

Next, Applicants explored the generalizability of CRISPR-mediated cleavage in eukaryotic cells by targeting additional protospacers within the EMX1 locus (FIG. 24A). To improve co-delivery, Applicants designed an expression vector to drive both pre-crRNA and SpCas9 (FIG. 31). In parallel, Applicants adapted a chimeric crRNA-tracrRNA hybrid (FIG. 24B, top) design recently validated in vitro (M. Jinek et al., A programmable dual-RNA-guided DNA endonuclease in adaptive bacterial immunity. Science 337, 816 (Aug. 17, 2012)), where a mature crRNA is fused to a partial tracrRNA via a synthetic stem-loop to mimic the natural crRNA:tracrRNA duplex (FIG. 24B, bottom). Applicants observed cleavage of all protospacer targets when SpCas9 is co-expressed with pre-crRNA (DR-spacer-DR) and tracrRNA. However, not all chimeric RNA designs could facilitate cleavage of their genomic targets (FIG. 24C, Table 1). Applicants then tested targeting of additional genomic loci in both human and mouse cells by designing pre-crRNAs and chimeric RNAs targeting the human PVALB and the mouse Th loci (FIG. 32). Applicants achieved efficient modification at all three mouse Th and one PVALB targets using the crRNA:tracrRNA design, thus demonstrating the broad applicability of the CRISPR system in modifying different loci across multiple organisms (Table 1). For the same protospacer targets, cleavage efficiencies of chimeric RNAs were either lower than those of crRNA:tracrRNA duplexes or undetectable. This may be due to differences in the expression and stability of RNAs, degradation by endogenous RNAi machinery, or secondary structures leading to inefficient Cas9 loading or target recognition.

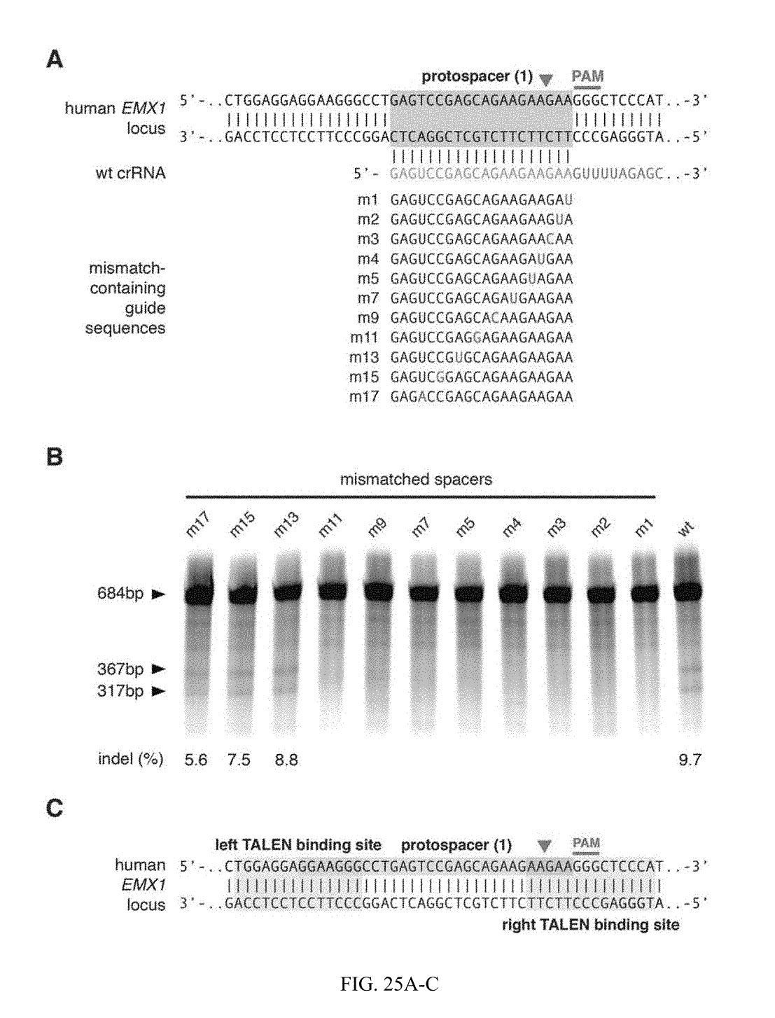

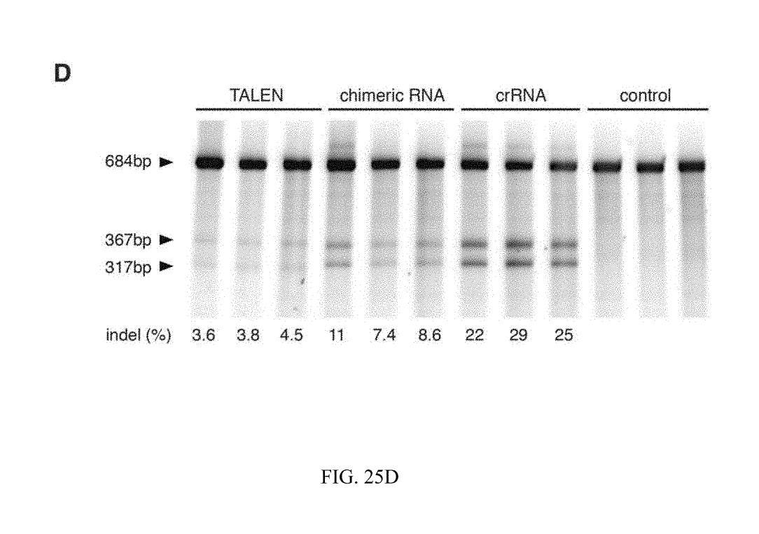

Effective genome editing requires that nucleases target specific genomic loci with both high precision and efficiency. To investigate the specificity of CRISPR-mediated cleavage, Applicants analyzed single-nucleotide mismatches between the spacer and its mammalian protospacer target (FIG. 25A). Applicants observed that single-base mismatch up to 12-bp 5′ of the PAM completely abolished genomic cleavage by SpCas9, whereas spacers with mutations farther upstream retained activity against the protospacer target (FIG. 25B). This is consistent with previous bacterial and in vitro studies of Cas9 specificity (M. Jinek et al., A programmable dual-RNA-guided DNA endonuclease in adaptive bacterial immunity. Science 337, 816 (Aug. 17, 2012) and R. Sapranauskas et al., The Streptococcus thermophilus CRISPR/Cas system provides immunity in Escherichia coli. Nucleic Acids Res 39, 9275 (November, 2011)). Furthermore, CRISPR is able to mediate genomic cleavage as efficiently as a pair of TALE nucleases (TALEN) targeting the same EMX1 protospacer (FIGS. 25, C and D).

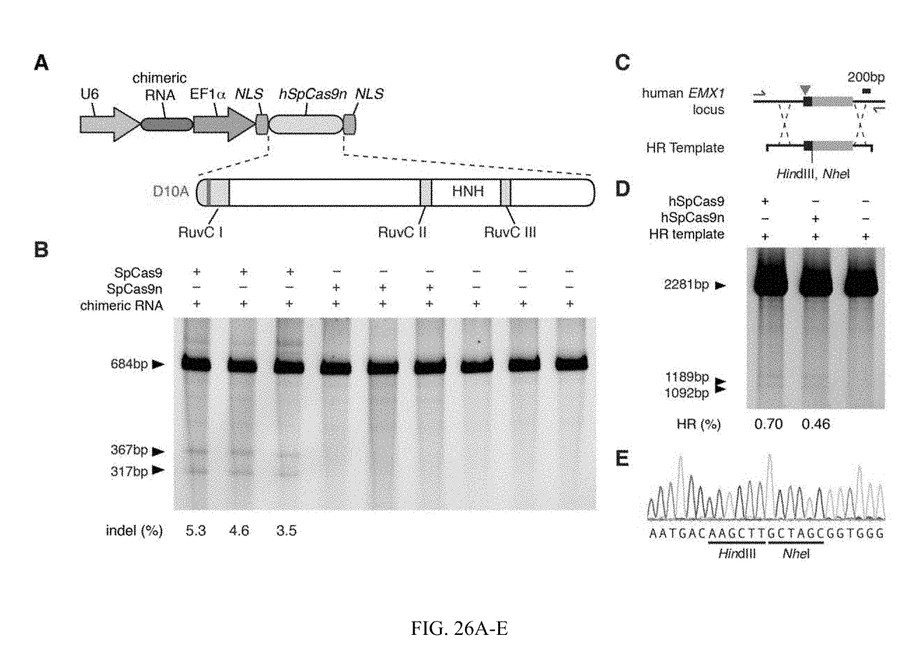

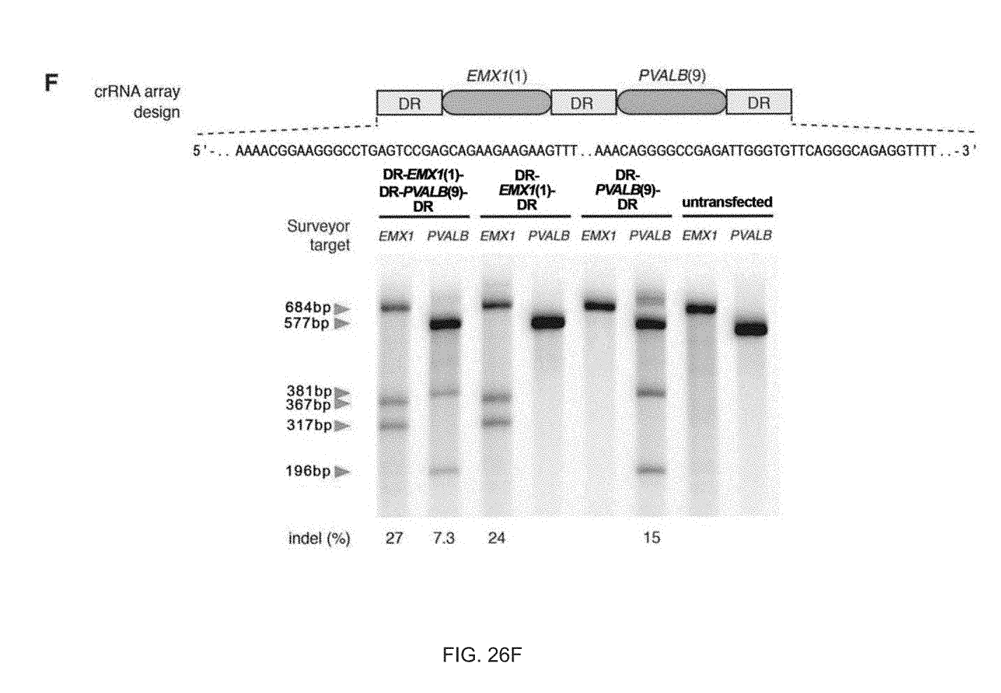

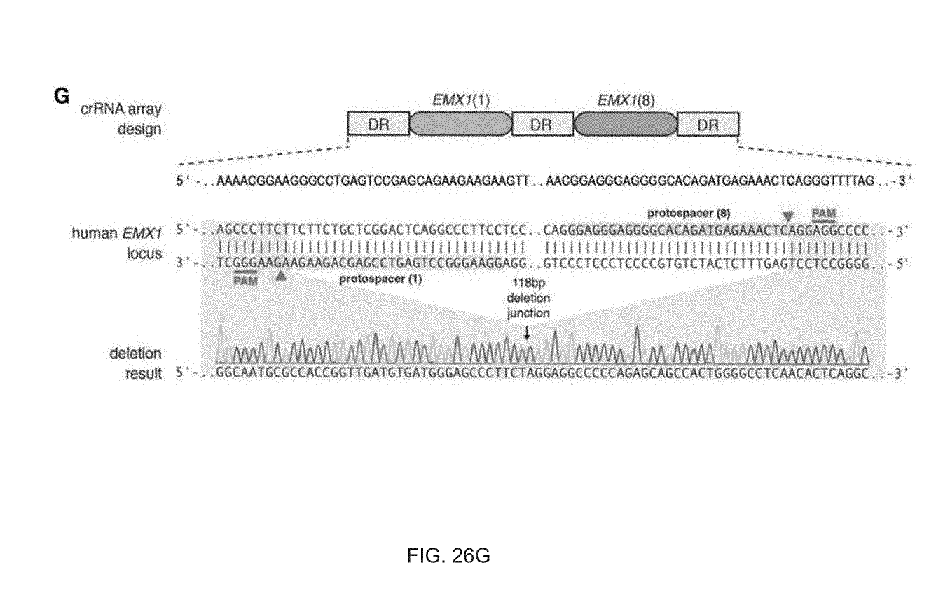

Targeted modification of genomes ideally avoids mutations arising from the error-prone NHEJ mechanism. The wild-type SpCas9 is able to mediate site-specific DSBs, which can be repaired through either NHEJ or homology-directed repair (HDR). Applicants engineered an aspartate-to-alanine substitution (D10A) in the RuvC I domain of SpCas9 to convert the nuclease into a DNA nickase (SpCas9n, FIG. 26A) (M. Jinek et al., A programmable dual-RNA-guided DNA endonuclease in adaptive bacterial immunity. Science 337, 816 (Aug. 17, 2012); G. Gasiunas, R. Barrangou, P. Horvath, V. Siksnys, Cas9-crRNA ribonucleoprotein complex mediates specific DNA cleavage for adaptive immunity in bacteria. Proc Natl Acad Sci USA 109, E2579 (Sep. 25, 2012) and R. Sapranauskas et al., The Streptococcus thermophilus CRISPR/Cas system provides immunity in Escherichia coli. Nucleic Acids Res 39, 9275 (November, 2011)), because nicked genomic DNA is typically repaired either seamlessly or through high-fidelity HDR. SURVEYOR (FIG. 26B) and sequencing of 327 amplicons did not detect any indels induced by SpCas9n. However, it is worth noting that nicked DNA can in rare cases be processed via a DSB intermediate and result in a NHEJ event (M. T. Certo et al., Tracking genome engineering outcome at individual DNA breakpoints. Nat Methods 8, 671 (August, 2011)). Applicants then tested Cas9-mediated HDR at the same EMX1 locus with a homology repair template to introduce a pair of restriction sites near the protospacer (FIG. 26C). SpCas9 and SpCas9n catalyzed integration of the repair template into EMX1 locus at similar levels (FIG. 26D), which Applicants further verified via Sanger sequencing (FIG. 26E). These results demonstrate the utility of CRISPR for facilitating targeted genomic insertions. Given the 14-bp (12-bp from the seed sequence and 2-bp from PAM) target specificity (FIG. 25B) of the wild type SpCas9, the use of a nickase may reduce off-target mutations.

Finally, the natural architecture of CRISPR loci with arrayed spacers (FIG. 27) suggests the possibility of multiplexed genome engineering. Using a single CRISPR array encoding a pair of EMX1- and PVALB-targeting spacers, Applicants detected efficient cleavage at both loci (FIG. 26F). Applicants further tested targeted deletion of larger genomic regions through concurrent DSBs using spacers against two targets within EMX1 spaced by 119-bp, and observed a 1.6% deletion efficacy (3 out of 182 amplicons; FIG. 26G), thus demonstrating the CRISPR system can mediate multiplexed editing within a single genome.

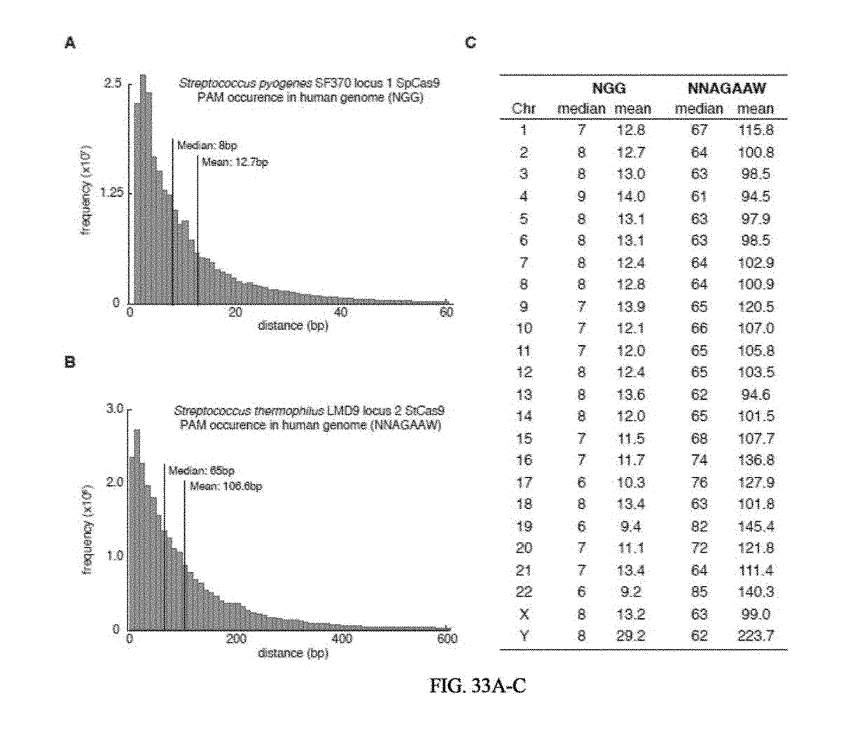

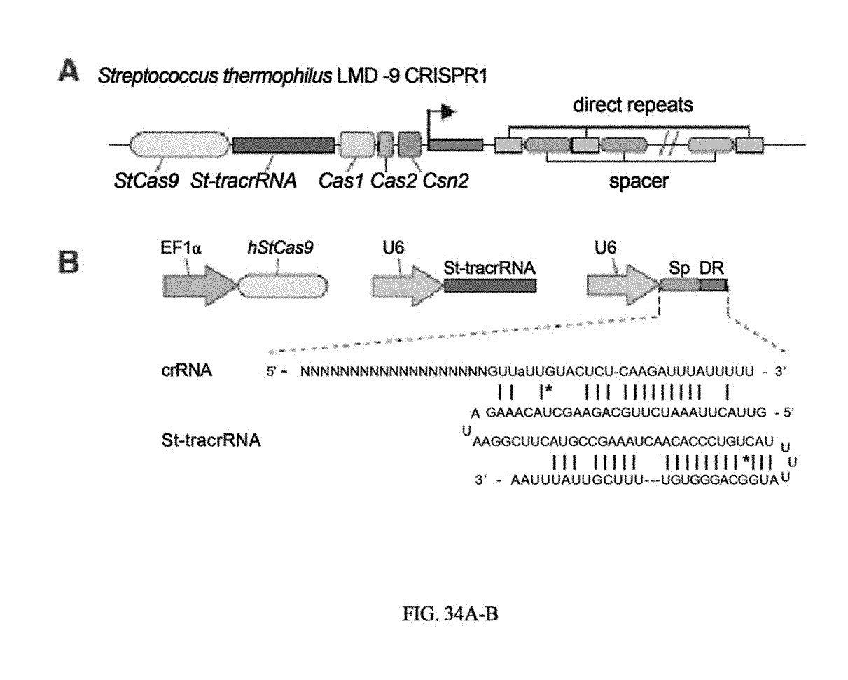

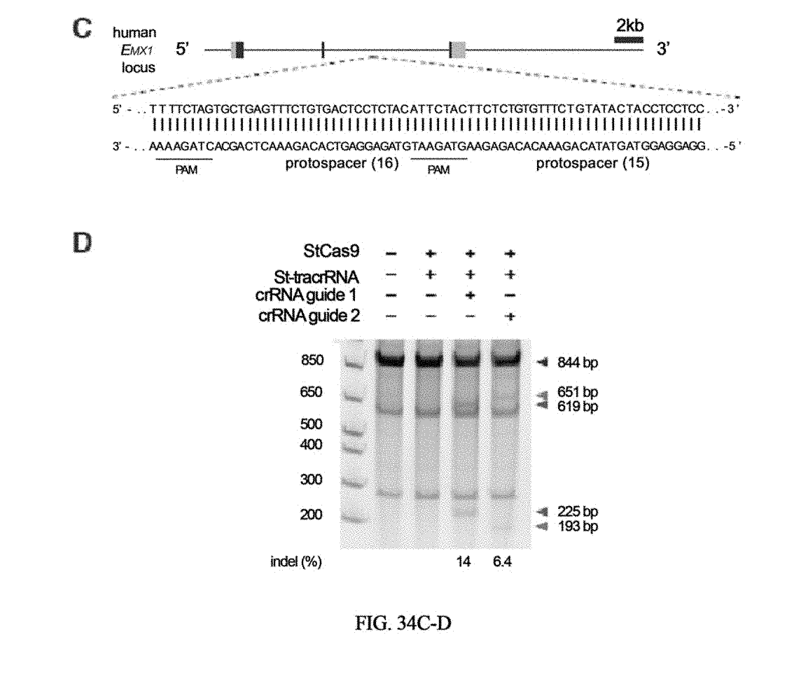

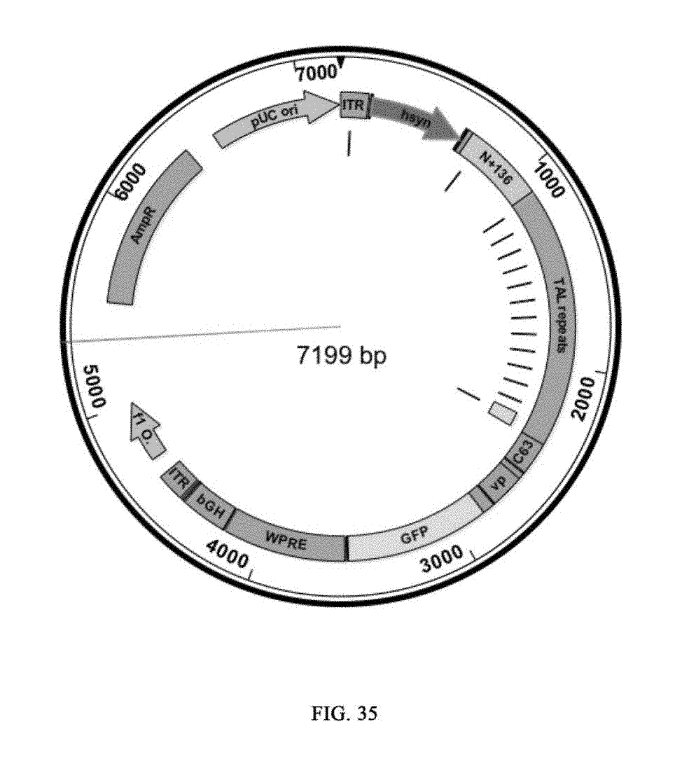

The ability to use RNA to program sequence-specific DNA cleavage defines a new class of genome engineering tools. Here, Applicants have shown that the S. pyogenes CRISPR system can be heterologously reconstituted in mammalian cells to facilitate efficient genome editing; an accompanying study has independently confirmed high efficiency CRISPR-mediated genome targeting in several human cell lines (Mali et al.). However, several aspects of the CRISPR system can be further improved to increase its efficiency and versatility. The requirement for an NGG PAM restricts the S. pyogenes CRISPR target space to every 8-bp on average in the human genome (FIG. 33), not accounting for potential constraints posed by crRNA secondary structure or genomic accessibility due to chromatin and DNA methylation states. Some of these restrictions may be overcome by exploiting the family of Cas9 enzymes and its differing PAM requirements (H. Deveau et al., Phage response to CRISPR-encoded resistance in Streptococcus thermophilus. J Bacteriol 190, 1390 (February, 2008) and F. J. Mojica, C. Diez-Villasenor, J. Garcia-Martinez, C. Almendros, Short motif sequences determine the targets of the prokaryotic CRISPR defence system. Microbiology 155, 733 (March, 2009)) across the microbial diversity (K. S. Makarova et al., Evolution and classification of the CRISPR-Cas systems. Nat Rev Microbiol 9, 467 (June, 2011)). Indeed, other CRISPR loci are likely to be transplantable into mammalian cells; for example, the Streptococcus thermophilus LMD-9 CRISPR1 can also mediate mammalian genome cleavage (FIG. 34). Finally, the ability to carry out multiplex genome editing in mammalian cells enables powerful applications across basic science, biotechnology, and medicine (P. A. Carr, G. M. Church, Genome engineering. Nat Biotechnol 27, 1151 (December, 2009)).

### Example 8

### Multiplex Genome Engineering Using CRISPR/Cas Systems: Supplementary Material

Cell Culture and Transfection.

Human embryonic kidney (HEK) cell line 293FT (Life Technologies) was maintained in Dulbecco's modified Eagle's Medium (DMEM) supplemented with 10% fetal bovine serum (HyClone), 2 mM GlutaMAX (Life Technologies), 100U/mL penicillin, and 100 g/mL streptomycin at 37° C. with 5% CO2 incubation. Mouse neuro2A (N2A) cell line (ATCC) was maintained with DMEM supplemented with 5% fetal bovine serum (HyClone), 2 mM GlutaMAX (Life Technologies), 100U/mL penicillin, and 100 g/mL streptomycin at 37° C. with 5% CO₂.

293FT or N2A cells were seeded into 24-well plates (Corning) one day prior to transfection at a density of 200,000 cells per well. Cells were transfected using Lipofectamine 2000 (Life Technologies) following the manufacturer's recommended protocol. For each well of a 24-well plate a total of 800ng plasmids was used.

Suveryor Assay and Sequencing Analysis for Genome Modification.

293FT or N2A cells were transfected with plasmid DNA as described above. Cells were incubated at 37° C. for 72 hours post transfection before genomic DNA extraction. Genomic DNA was extracted using the QuickExtract DNA extraction kit (Epicentre) following the manufacturer's protocol. Briefly, cells were resuspended in QuickExtract solution and incubated at 65° C. for 15 minutes and 98° C. for 10 minutes.

Genomic region surrounding the CRISPR target site for each gene was PCR amplified, and products were purified using QiaQuick Spin Column (Qiagen) following manufacturer's protocol. A total of 400ng of the purified PCR products were mixed with 2 μl 10X Taq polymerase PCR buffer (Enzymatics) and ultrapure water to a final volume of 20 μl, and subjected to a re-annealing process to enable heteroduplex formation: 95° C. for 10 min, 95C to 85° C. ramping at −2° C./s, 85° C. to 25° C. at −0.25° C./s, and 25° C. hold for 1 minute. After reannealing, products were treated with SURVEYOR nuclease and SURVEYOR enhancer S (Transgenomics) following the manufacturer's recommended protocol, and analyzed on 4-20 Novex TBE poly-acrylamide gels (Life Technologies). Gels were stained with SYBR Gold DNA stain (Life Technologies) for 30 minutes and imaged with a Gel Doc gel imaging system (Biorad). Quantification was based on relative band intensities.

Restriction Fragment Length Polymorphism Assay for Detection of Homologous Recombination.

HEK 293FT and N2A cells were transfected with plasmid DNA, and incubated at 37° C. for 72 hours before genomic DNA extraction as described above. The target genomic region was PCR amplified using primers outside the homology arms of the homologous recombination (HR) template. PCR products were separated on a 1% agarose gel and extracted with MinElute GelExtraction Kit (Qiagen). Purified products were digested with HindIII (Fermentas) and analyzed on a 6% Novex TBE poly-acrylamide gel (Life Technologies).

RNA Extraction and Purification.

HEK 293FT cells were maintained and transfected as stated previously. Cells were harvested by trypsinization followed by washing in phosphate buffered saline (PBS). Total cell RNA was extracted with TRI reagent (Sigma) following manufacturer's protocol. Extracted total RNA was quantified using Naonodrop (Thermo Scientific) and normalized to same concentration.

Northern Blot Analysis of crRNA and tracrRNA Expression in Mammalian Cells.

RNAs were mixed with equal volumes of 2X loading buffer (Ambion), heated to 95° C. for 5 min, chilled on ice for 1 min and then loaded onto 8% denaturing polyacrylamide gels (SequaGel, National Diagnostics) after pre-running the gel for at least 30 minutes. The samples were electrophoresed for 1.5 hours at 40 W limit. Afterwards, the RNA was transferred to Hybond N+ membrane (GE Healthcare) at 300 mA in a semi-dry transfer apparatus (Bio-rad) at room temperature for 1.5 hours. The RNA was crosslinked to the membrane using autocrosslink button on Stratagene UV Crosslinker the Stratalinker (Stratagene). The membrane was pre-hybridized in ULTRAhyb-Oligo Hybridization Buffer (Ambion) for 30 min with rotation at 42° C. and then probes were added and hybridized overnight. Probes were ordered from IDT and labeled with [gamma-32P] ATP (Perkin Elmer) with T4 polynucleotide kinase (New England Biolabs). The membrane was washed once with pre-warmed (42° C.) 2×SSC, 0.5% SDS for 1 min followed by two 30 minute washes at 42° C. The membrane was exposed to phosphor screen for one hour or overnight at room temperature and then scanned with phosphorimager (Typhoon).

Table 1. Protospacer sequences and modification efficiencies of mammalian genomic targets. Protospacer targets designed based on Streptococcus pyogenes type II CRISPR and Streptococcus thermophilus CRISPR1 loci with their requisite PAMs against three different genes in human and mouse genomes. Cells were transfected with Cas9 and either precrRNA/tracrRNA or chimeric RNA. Cells were analyzed 72 hours after transfection. Percent indels are calculated based on SURVEYOR assay results from indicated cell lines, N=3 for all protospacer targets, errors are S.E.M. N.D., not detectable using the SURVEYOR assay; N.T., not tested in this study. Table 1 discloses SEQ ID NOS 46-61, respectively, in order of appearance.

| | target | | protospacer | | |
|---|---|---|---|---|---|
| Cas9 | species | gene | ID | protospacer sequence (5′ to 3′) | PAM |
| S. pyogenes | Homo | EMX1 | 1 | GGAAGGGCCTGAGTCCGAGCAGAAGAAGAA | GGG |
| SF370 type II | sapiens | EMX1 | 2 | CATTGGAGGTGACATCGATGTCCTCCCCAT | TGG |
| CRISPR | | EMX1 | 3 | GGACATCGATGTCACCTCCAATGACTAGGG | TGG |
| | | EMX1 | 4 | CATCGATGTCCTCCCCATTGGCCTGCTTCG | TGG |
| | | EMX1 | 5 | TTCGTGGCAATGCGCCACCGGTTGATGTGAT | TGG |
| | | EMX1 | 6 | TCGTGGCAATGCGCCACCGGTTGATGTGAT | GGG |
| | | EMX1 | 7 | TCCAGCTTCTGCCGTTTGTACTTTGTCCTC | CGG |
| | | EMX1 | 8 | GGAGGGAGGGGCACAGATGAGAAACTCAGG | AGG |
| | Homo | PVALB | 9 | AGGGGCCGAGATTGGGTGTTCAGGGCAGAG | AGG |
| | sapiens | PVALB | 10 | ATGCAGGAGGGTGGCGAGAGGGGCCGAGAT | TGG |
| | | PVALB | 11 | GGTGGCGAGAGGGGCCGAGATTGGGTGTTC | AGG |
| | Mus | Th | 12 | CAAGCACTGAGTGCCATTAGCTAAATGCAT | AGG |
| | musculus | Th | 13 | AATGCATAGGGTACCACCCACAGGTGCCAG | GGG |
| | | Th | 14 | ACACACATGGGAAAGCCTCTGGGCCAGGAA | AGG |
| S. thermophilus | Homo | EMX1 | 15 | GGAGGAGGTAGTATACAGAAACACAGAGAA | GTAGAAT |
| LMD-9 CRISPR1 | sapiens | EMX1 | 16 | AGAATGTAGAGGAGTCACAGAAACTCAGCA | CTAGAAA |

| | | | | | | % Indol | % Indol |
|---|---|---|---|---|---|---|---|
| | target | | protospacer | | cell line | (pre-crRNA + | (chimeric |
| Cas9 | species | gene | ID | strand | tested | tracrRNA) | RNA) |
| S. pyogenes | Homo | EMX1 | 1 | + | 293FT | 20 ± 1.6 | 6.7 ± 0.62 |
| SF370 type II | sapiens | EMX1 | 2 | − | 293FT | 2.1 ± 0.31 | N.D. |
| CRISPR | | EMX1 | 3 | + | 293FT | 14 ± 1.1 | N.D. |
| | | EMX1 | 4 | − | 293FT | 11 ± 1.7 | N.D. |
| | | EMX1 | 5 | − | 293FT | 4.3 ± 0.46 | 2.1 ± 0.51 |
| | | EMX1 | 6 | − | 293FT | 4.0 ± 0.60 | 0.41 ± 0.25 |
| | | EMX1 | 7 | − | 293FT | 1.5 ± 0.12 | N.D. |
| | | EMX1 | 8 | − | 293FT | 7.8 ± 0.83 | 2.3 ± 1.2 |
| | Homo | PVALB | 9 | + | 293FT | 21 ± 2.6 | 8.5 ± 0.32 |
| | sapiens | PVALB | 10 | + | 293FT | N.D. | N.D. |
| | | PVALB | 11 | + | 293FT | N.D. | N.D. |
| | Mus | Th | 12 | − | Neuro2A | 27 ± 4.3 | 4.1 ± 2.2 |
| | musculus | Th | 13 | − | Neuro2A | 4.8 ± 1.2 | N.D. |
| | | Th | 14 | + | Neuro2A | 11.3 ± 1.3 | N.D. |
| S. | Homo | EMX1 | 15 | − | 293FT | 14 ± 0.86 | N.T. |
| thermophilus | | | | | | | |
| LMD-9 CRISPR1 | sapiens | EMX1 | 16 | − | 293FT | 7.8 ± 0.77 | N.T. |

TABLE 2
Sequences for primers and probes (SEQ ID NOS 62-73, respectively, in
order of appearance) used for SURVEYOR assay, RFLP assay,
genomic sequencing, and Northern blot.

| | | Genomic | |
|---|---|---|---|
| Primer name | Assay | Target | Primer sequence |
| Sp-EMX1-F | SURVEYOR | EMX1 | AAAACCACCCTTCTCTCTGGC |
| | assay, sequencing | | |
| Sp-EMX1-R | SURVEYOR | EMX1 | GGAGATTGGAGACACGGAGAG |
| | assay, sequencing | | |
| Sp-PVALB-F | SURVEYOR | PVALB | CTGGAAAGCCAATGCCTGAC |
| | assay, sequencing | | |
| Sp-PVALB-R | SURVEYOR | PVALB | GGCAGCAAACTCCTTGTCCT |
| | assay, sequencing | | |
| Sp-Th-F | SURVEYOR | Th | GTGCTTTGCAGAGGCCTACC |
| | assay, sequencing | | |
| Sp-Th-R | SURVEYOR | Th | CCTGGAGCGCATGCAGTAGT |
| | assay, sequencing | | |
| St-EMX1-F | SURVEYOR | EMX1 | ACCTTCTGTGTTTCCACCATTC |
| | assay, sequencing | | |
| St-EMX1-R | SURVEYOR | EMX1 | TTGGGGAGTGCACAGACTTC |
| | assay, sequencing | | |
| Sp-EMX1- | RFLP, | EMX1 | GGCTCCCTGGGTTCAAAGTA |
| RFLP-F | sequencing | | |
| Sp-EMX1- | RFLP, | EMX1 | AGAGGGGTCTGGATGTCGTAA |
| RFLP-R | sequencing | | |
| Pb_EMX1_sp1 | Northern Blot | Not | TAGCTCTAAAACTTCTTCTTCTGCTCGGAC |
| | Probe | applicable | |
| Pb_tracrRNA | Northern Blot | Not | CTAGCCTTATTTTAACTTGCTATGCTGTTT |
| | Probe | applicable | |

Supplementary Sequences

> U6-short tracrRNA (Streptococcus pyogenes SF370)
(SEQ ID NO: 74)
GAGGGCCTATTTCCCATGATTCCTTCATATTTGCATATACGATACAAGGCT
GTTAGAGAGATAATTGGAATTAATTTGACTGTAAACACAAAGATATTAGTACAAAA
TACGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTTAT
ATATCTTGTGGAAAGGACGAAACACCGGAACCATTCAAAACAGCATAGCAAGTTAA
AATAAGGCTAGTCCGTTATCAACTTGAAAAAGTGGCACCGAGTCGGTGCTTTTTTT
> U6-long tracrRNA (Streptococcus pyogenes SF370)
(SEQ ID NO: 75)
GAGGGCCTATTTCCCATGATTCCTTCATATTTGCATATACGATACAAGGCT
GTTAGAGAGATAATTGGAATTAATTTGACTGTAAACACAAAGATATTAGTACAAAA
TACGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTTAT
ATATCTTGTGGAAAGGACGAAACACCGGTAGTATTAAGTATTGTTTTATGGCTGATA
AATTTCTTTGAATTTCTCCTTGATTATTTGTTATAAAAGTTATAAAATAATCTTGTTG
GAACCATTCAAAACAGCATAGCAAGTTAAAATAAGGCTAGTCCGTTATCAACTTGA
AAAAGTGGCACCGAGTCGGTGCTTTTTTT
> U6-DR-BbsI backbone-DR (Streptococcus pyogenes SF370)
(SEQ ID NO: 76)
GAGGGCCTATTTCCCATGATTCCTTCATATTTGCATATACGATACAAGGCT
GTTAGAGAGATAATTGGAATTAATTTGACTGTAAACACAAAGATATTAGTACAAAA
TACGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTTAT
ATATCTTGTGGAAAGGACGAAACACCGGGTTTTAGAGCTATGCTGTTTTGAATGGTC
CCAAAACGGGTCTTCGAGAAGACGTTTTAGAGCTATGCTGTTTTGAATGGTCCCAAA
AC
> U6-chimeric RNA-BbsI backbone (Streptococcus pyogenes
SF370)
(SEQ ID NO: 77)
GAGGGCCTATTTCCCATGATTCCTTCATATTTGCATATACGATACAAGGCT
GTTAGAGAGATAATTGGAATTAATTTGACTGTAAACACAAAGATATTAGTACAAAA
TACGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTTAT
ATATCTTGTGGAAAGGACGAAACACCGGGTCTTCGAGAAGACCTGTTTTAGAGCTA
GAAATAGCAAGTTAAAATAAGGCTAGTCCG
> 3xFLAG-NLS-SpCas9-NLS
(SEQ ID NO: 78)
ATGGACTATAAGGACCACGACGGAGACTACAAGGATCATGATATTGATTA
CAAAGACGATGACGATAAGATGGCCCCAAAGAAGAAGCGGAAGGTCGGTATCCAC
GGAGTCCCAGCAGCCGACAAGAAGTACAGCATCGGCCTGGACATCGGCACCAACTC
TGTGGGCTGGGCCGTGATCACCGACGAGTACAAGGTGCCCAGCAAGAAATTCAAGG
TGCTGGGCAACACCGACCGGCACAGCATCAAGAAGAACCTGATCGGAGCCCTGCTG
TTCGACAGCGGCGAAACAGCCGAGGCCACCCGGCTGAAGAGAACCGCCAGAAGAA
GATACACCAGACGGAAGAACCGGATCTGCTATCTGCAAGAGATCTTCAGCAACGAG
ATGGCCAAGGTGGACGACAGCTTCTTCCACAGACTGGAAGAGTCCTTCCTGGTGGA
AGAGGATAAGAAGCACGAGCGGCACCCCATCTTCGGCAACATCGTGGACGAGGTGG
CCTACCACGAGAAGTACCCCACCATCTACCACCTGAGAAAGAAACTGGTGGACAGC
ACCGACAAGGCCGACCTGCGGCTGATCTATCTGGCCCTGGCCCACATGATCAAGTTC
CGGGGCCACTTCCTGATCGAGGGCGACCTGAACCCCGACAACAGCGACGTGGACAA
GCTGTTCATCCAGCTGGTGCAGACCTACAACCAGCTGTTCGAGGAAAACCCCATCAA
CGCCAGCGGCGTGGACGCCAAGGCCATCCTGTCTGCCAGACTGAGCAAGAGCAGAC
GGCTGGAAAATCTGATCGCCCAGCTGCCCGGCGAGAAGAAGAATGGCCTGTTCGGC
AACCTGATTGCCCTGAGCCTGGGCCTGACCCCCAACTTCAAGAGCAACTTCGACCTG
GCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCT
GTCCGACGCCATCCTGCTGAGCGACATCCTGAGAGTGAACACCGAGATCACCAAGG
CCCCCCTGAGCGCCTCTATGATCAAGAGATACGACGAGCACCACCAGGACCTGACC
CTGCTGAAAGCTCTCGTGCGGCAGCAGCTGCCTGAGAAGTACAAAGAGATTTTCTTC
GACCAGAGCAAGAACGGCTACGCCGGCTACATTGACGGCGGAGCCAGCCAGGAAG
AGTTCTACAAGTTCATCAAGCCCATCCTGGAAAAGATGGACGGCACCGAGGAACTG
CTCGTGAAGCTGAACAGAGAGGACCTGCTGCGGAAGCAGCGGACCTTCGACAACGG
CAGCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTGG
ATGACCAGAAAGAGCGAGGAAACCATCACCCCCTGGAACTTCGAGGAAGTGGTGGA
CAAGGGCGCTTCCGCCCAGAGCTTCATCGAGCGGATGACCAACTTCGATAAGAACC
TGCCCAACGAGAAGGTGCTGCCCAAGCACAGCCTGCTGTACGAGTACTTCACCGTGT
ATAACGAGCTGACCAAAGTGAAATACGTGACCGAGGGAATGAGAAAGCCCGCCTTC
CTGAGCGGCGAGCAGAAAAAGGCCATCGTGGACCTGCTGTTCAAGACCAACCGGAA
AGTGACCGTGAAGCAGCTGAAAGAGGACTACTTCAAGAAAATCGAGTGCTTCGACT
CCGTGGAAATCTCCGGCGTGGAAGATCGGTTCAACGCCTCCCTGGGCACATACCAC
GATCTGCTGAAAATTATCAAGGACAAGGACTTCCTGGACAATGAGGAAAACGAGGA
CATTCTGGAAGATATCGTGCTGACCCTGACACTGTTTGAGGACAGAGAGATGATCGA
GGAACGGCTGAAAACCTATGCCCACCTGTTCGACGACAAAGTGATGAAGCAGCTGA
AGCGGCGGAGATACACCGGCTGGGGCAGGCTGAGCCGGAAGCTGATCAACGGCATC
CGGGACAAGCAGTCCGGCAAGACAATCCTGGATTTCCTGAAGTCCGACGGCTTCGC
CAACAGAAACTTCATGCAGCTGATCCACGACGACAGCCTGACCTTTAAAGAGGACA
TCCAGAAAGCCCAGGTGTCCGGCCAGGGCGATAGCCTGCACGAGCACATTGCCAAT
CTGGCCGGCAGCCCCGCCATTAAGAAGGGCATCCTGCAGACAGTGAAGGTGGTGGA
CGAGCTCGTGAAAGTGATGGGCCGGCACAAGCCCGAGAACATCGTGATCGAAATGG
CCAGAGAGAACCAGACCACCCAGAAGGGACAGAAGAACAGCCGCGAGAGAATGAA
GCGGATCGAAGAGGGCATCAAAGAGCTGGGCAGCCAGATCCTGAAAGAACACCCC
GTGGAAAACACCCAGCTGCAGAACGAGAAGCTGTACCTGTACTACCTGCAGAATGG
GCGGGATATGTACGTGGACCAGGAACTGGACATCAACCGGCTGTCCGACTACGATG
TGGACCATATCGTGCCTCAGAGCTTTCTGAAGGACGACTCCATCGACAACAAGGTGC
TGACCAGAAGCGACAAGAACCGGGGCAAGAGCGACAACGTGCCCTCCGAAGAGGT
CGTGAAGAAGATGAAGAACTACTGGCGGCAGCTGCTGAACGCCAAGCTGATTACCC
AGAGAAAGTTCGACAATCTGACCAAGGCCGAGAGAGGCGGCCTGAGCGAACTGGAT
AAGGCCGGCTTCATCAAGAGACAGCTGGTGGAAACCCGGCAGATCACAAAGCACGT
GGCACAGATCCTGGACTCCCGGATGAACACTAAGTACGACGAGAATGACAAGCTGA
TCCGGGAAGTGAAAGTGATCACCCTGAAGTCCAAGCTGGTGTCCGATTTCCGGAAG
GATTTCCAGTTTTACAAAGTGCGCGAGATCAACAACTACCACCACGCCCACGACGCC
TACCTGAACGCCGTCGTGGGAACCGCCCTGATCAAAAAGTACCCTAAGCTGGAAAG
CGAGTTCGTGTACGGCGACTACAAGGTGTACGACGTGCGGAAGATGATCGCCAAGA
GCGAGCAGGAAATCGGCAAGGCTACCGCCAAGTACTTCTTCTACAGCAACATCATG
AACTTTTTCAAGACCGAGATTACCCTGGCCAACGGCGAGATCCGGAAGCGGCCTCT
GATCGAGACAAACGGCGAAACCGGGGAGATCGTGTGGGATAAGGGCCGGGATTTTG
CCACCGTGCGGAAAGTGCTGAGCATGCCCCAAGTGAATATCGTGAAAAAGACCGAG
GTGCAGACAGGCGGCTTCAGCAAAGAGTCTATCCTGCCCAAGAGGAACAGCGATAA
GCTGATCGCCAGAAAGAAGGACTGGGACCCTAAGAAGTACGGCGGCTTCGACAGCC
CCACCGTGGCCTATTCTGTGCTGGTGGTGGCCAAAGTGGAAAAGGGCAAGTCCAAG
AAACTGAAGAGTGTGAAAGAGCTGCTGGGGATCACCATCATGGAAAGAAGCAGCTT
CGAGAAGAATCCCATCGACTTTCTGGAAGCCAAGGGCTACAAAGAAGTGAAAAAGG
ACCTGATCATCAAGCTGCCTAAGTACTCCCTGTTCGAGCTGGAAAACGGCCGGAAG
AGAATGCTGGCCTCTGCCGGCGAACTGCAGAAGGGAAACGAACTGGCCCTGCCCTC
CAAATATGTGAACTTCCTGTACCTGGCCAGCCACTATGAGAAGCTGAAGGGCTCCCC
CGAGGATAATGAGCAGAAACAGCTGTTTGTGGAACAGCACAAGCACTACCTGGACG
AGATCATCGAGCAGATCAGCGAGTTCTCCAAGAGAGTGATCCTGGCCGACGCTAAT
CTGGACAAAGTGCTGTCCGCCTACAACAAGCACCGGGATAAGCCCATCAGAGAGCA
GGCCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGTGC
TGGACGCCACCCTGATCCACCAGAGCATCACCGGCCTGTACGAGACACGGATCGAC
CTGTCTCAGCTGGGAGGCGACAAGCGTCCTGCTGCTACTAAGAAAGCTGGTCAAGCT
AAGAAAAAGAAA
> SpRNase3-mCherry-NLS
(SEQ ID NO: 79)
ATGAAGCAGCTGGAGGAGTTACTTTCTACCTCTTTCGACATCCAGTTTAAT
GACCTGACCCTGCTGGAAACCGCCTTCACTCACACCTCCTACGCGAATGAGCACCGC
CTACTGAATGTGAGCCACAACGAGCGCCTGGAGTTTCTGGGGGATGCTGTCTTACAG
CTGATCATCTCTGAATATCTGTTTGCCAAATACCCTAAGAAAACCGAAGGGGACATG
TCAAAGCTGCGCTCCATGATAGTCAGGGAAGAGAGCCTGGCGGGCTTTAGTCGTTTT
TGCTCATTCGACGCTTATATCAAGCTGGGAAAAGGCGAAGAGAAGTCCGGCGGCAG
GAGGCGCGATACAATTCTGGGCGATCTCTTTGAAGCGTTTCTGGGCGCACTTCTACT
GGACAAAGGGATCGACGCAGTCCGCCGCTTTCTGAAACAAGTGATGATCCCTCAGG
TCGAAAAGGGAAACTTCGAGAGAGTGAAGGACTATAAAACATGTTTGCAGGAATTT
CTCCAGACCAAGGGAGATGTAGCAATAGATTATCAGGTAATAAGTGAGAAAGGACC
AGCTCACGCCAAACAATTCGAAGTTAGCATCGTTGTTAATGGCGCAGTGTTGTCGAA
GGGCTTGGGTAAATCAAAAAAACTGGCCGAGCAGGACGCTGCTAAAAACGCCCTCG
CTCAGCTCAGCGAGGTAGGATCCGTGAGCAAGGGCGAGGAGGATAACATGGCCATC
ATCAAGGAGTTCATGCGCTTCAAGGTGCACATGGAGGGCTCCGTGAACGGCCACGA
GTTCGAGATCGAGGGCGAGGGCGAGGGCCGCCCCTACGAGGGCACCCAGACCGCCA
AGCTGAAGGTGACCAAGGGTGGCCCCCTGCCCTTCGCCTGGGACATCCTGTCCCCTC
AGTTCATGTACGGCTCCAAGGCCTACGTGAAGCACCCCGCCGACATCCCCGACTACT
TGAAGCTGTCCTTCCCCGAGGGCTTCAAGTGGGAGCGCGTGATGAACTTCGAGGAC
GGCGGCGTGGTGACCGTGACCCAGGACTCCTCCCTGCAGGACGGCGAGTTCATCTA
CAAGGTGAAGCTGCGCGGCACCAACTTCCCCTCCGACGGCCCCGTAATGCAGAAGA
AGACCATGGGCTGGGAGGCCTCCTCCGAGCGGATGTACCCCGAGGACGGCGCCCTG
AAGGGCGAGATCAAGCAGAGGCTGAAGCTGAAGGACGGCGGCCACTACGACGCTG
AGGTCAAGACCACCTACAAGGCCAAGAAGCCCGTGCAGCTGCCCGGCGCCTACAAC
GTCAACATCAAGTTGGACATCACCTCCCACAACGAGGACTACACCATCGTGGAACA
GTACGAACGCGCCGAGGGCCGCCACTCCACCGGCGGCATGGACGAGCTGTACAAGA
AGCGTCCTGCTGCTACTAAGAAAGCTGGTCAAGCTAAGAAAAAGAAA
> 3xFLAG-NLS-SpCas9n-NLS (the D10A nickase
mutation is underlined)
(SEQ ID NO: 80)
ATGGACTATAAGGACCACGACGGAGACTACAAGGATCATGATATTGATTA
CAAAGACGATGACGATAAGATGGCCCCAAAGAAGAAGCGGAAGGTCGGTATCCAC
GGAGTCCCAGCAGCCGACAAGAAGTACAGCATCGGCCTGGCCATCGGCACCAACTC
TGTGGGCTGGGCCGTGATCACCGACGAGTACAAGGTGCCCAGCAAGAAATTCAAGG
TGCTGGGCAACACCGACCGGCACAGCATCAAGAAGAACCTGATCGGAGCCCTGCTG
TTCGACAGCGGCGAAACAGCCGAGGCCACCCGGCTGAAGAGAACCGCCAGAAGAA
GATACACCAGACGGAAGAACCGGATCTGCTATCTGCAAGAGATCTTCAGCAACGAG
ATGGCCAAGGTGGACGACAGCTTCTTCCACAGACTGGAAGAGTCCTTCCTGGTGGA
AGAGGATAAGAAGCACGAGCGGCACCCCATCTTCGGCAACATCGTGGACGAGGTGG
CCTACCACGAGAAGTACCCCACCATCTACCACCTGAGAAAGAAACTGGTGGACAGC
ACCGACAAGGCCGACCTGCGGCTGATCTATCTGGCCCTGGCCCACATGATCAAGTTC
CGGGGCCACTTCCTGATCGAGGGCGACCTGAACCCCGACAACAGCGACGTGGACAA
GCTGTTCATCCAGCTGGTGCAGACCTACAACCAGCTGTTCGAGGAAAACCCCATCAA
CGCCAGCGGCGTGGACGCCAAGGCCATCCTGTCTGCCAGACTGAGCAAGAGCAGAC
GGCTGGAAAATCTGATCGCCCAGCTGCCCGGCGAGAAGAAGAATGGCCTGTTCGGC
AACCTGATTGCCCTGAGCCTGGGCCTGACCCCCAACTTCAAGAGCAACTTCGACCTG
GCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCT
GTCCGACGCCATCCTGCTGAGCGACATCCTGAGAGTGAACACCGAGATCACCAAGG
CCCCCCTGAGCGCCTCTATGATCAAGAGATACGACGAGCACCACCAGGACCTGACC
CTGCTGAAAGCTCTCGTGCGGCAGCAGCTGCCTGAGAAGTACAAAGAGATTTTCTTC
GACCAGAGCAAGAACGGCTACGCCGGCTACATTGACGGCGGAGCCAGCCAGGAAG
AGTTCTACAAGTTCATCAAGCCCATCCTGGAAAAGATGGACGGCACCGAGGAACTG
CTCGTGAAGCTGAACAGAGAGGACCTGCTGCGGAAGCAGCGGACCTTCGACAACGG
CAGCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTGG
ATGACCAGAAAGAGCGAGGAAACCATCACCCCCTGGAACTTCGAGGAAGTGGTGGA
CAAGGGCGCTTCCGCCCAGAGCTTCATCGAGCGGATGACCAACTTCGATAAGAACC
TGCCCAACGAGAAGGTGCTGCCCAAGCACAGCCTGCTGTACGAGTACTTCACCGTGT
ATAACGAGCTGACCAAAGTGAAATACGTGACCGAGGGAATGAGAAAGCCCGCCTTC
CTGAGCGGCGAGCAGAAAAAGGCCATCGTGGACCTGCTGTTCAAGACCAACCGGAA
AGTGACCGTGAAGCAGCTGAAAGAGGACTACTTCAAGAAAATCGAGTGCTTCGACT
CCGTGGAAATCTCCGGCGTGGAAGATCGGTTCAACGCCTCCCTGGGCACATACCAC
GATCTGCTGAAAATTATCAAGGACAAGGACTTCCTGGACAATGAGGAAAACGAGGA
CATTCTGGAAGATATCGTGCTGACCCTGACACTGTTTGAGGACAGAGAGATGATCGA
GGAACGGCTGAAAACCTATGCCCACCTGTTCGACGACAAAGTGATGAAGCAGCTGA
AGCGGCGGAGATACACCGGCTGGGGCAGGCTGAGCCGGAAGCTGATCAACGGCATC
CGGGACAAGCAGTCCGGCAAGACAATCCTGGATTTCCTGAAGTCCGACGGCTTCGC
CAACAGAAACTTCATGCAGCTGATCCACGACGACAGCCTGACCTTTAAAGAGGACA
TCCAGAAAGCCCAGGTGTCCGGCCAGGGCGATAGCCTGCACGAGCACATTGCCAAT
CTGGCCGGCAGCCCCGCCATTAAGAAGGGCATCCTGCAGACAGTGAAGGTGGTGGA
CGAGCTCGTGAAAGTGATGGGCCGGCACAAGCCCGAGAACATCGTGATCGAAATGG
CCAGAGAGAACCAGACCACCCAGAAGGGACAGAAGAACAGCCGCGAGAGAATGAA
GCGGATCGAAGAGGGCATCAAAGAGCTGGGCAGCCAGATCCTGAAAGAACACCCC
GTGGAAAACACCCAGCTGCAGAACGAGAAGCTGTACCTGTACTACCTGCAGAATGG
GCGGGATATGTACGTGGACCAGGAACTGGACATCAACCGGCTGTCCGACTACGATG
TGGACCATATCGTGCCTCAGAGCTTTCTGAAGGACGACTCCATCGACAACAAGGTGC
TGACCAGAAGCGACAAGAACCGGGGCAAGAGCGACAACGTGCCCTCCGAAGAGGT
CGTGAAGAAGATGAAGAACTACTGGCGGCAGCTGCTGAACGCCAAGCTGATTACCC
AGAGAAAGTTCGACAATCTGACCAAGGCCGAGAGAGGCGGCCTGAGCGAACTGGAT
AAGGCCGGCTTCATCAAGAGACAGCTGGTGGAAACCCGGCAGATCACAAAGCACGT
GGCACAGATCCTGGACTCCCGGATGAACACTAAGTACGACGAGAATGACAAGCTGA
TCCGGGAAGTGAAAGTGATCACCCTGAAGTCCAAGCTGGTGTCCGATTTCCGGAAG
GATTTCCAGTTTTACAAAGTGCGCGAGATCAACAACTACCACCACGCCCACGACGCC
TACCTGAACGCCGTCGTGGGAACCGCCCTGATCAAAAAGTACCCTAAGCTGGAAAG
CGAGTTCGTGTACGGCGACTACAAGGTGTACGACGTGCGGAAGATGATCGCCAAGA
GCGAGCAGGAAATCGGCAAGGCTACCGCCAAGTACTTCTTCTACAGCAACATCATG
AACTTTTTCAAGACCGAGATTACCCTGGCCAACGGCGAGATCCGGAAGCGGCCTCT
GATCGAGACAAACGGCGAAACCGGGGAGATCGTGTGGGATAAGGGCCGGGATTTTG
CCACCGTGCGGAAAGTGCTGAGCATGCCCCAAGTGAATATCGTGAAAAAGACCGAG
GTGCAGACAGGCGGCTTCAGCAAAGAGTCTATCCTGCCCAAGAGGAACAGCGATAA
GCTGATCGCCAGAAAGAAGGACTGGGACCCTAAGAAGTACGGCGGCTTCGACAGCC
CCACCGTGGCCTATTCTGTGCTGGTGGTGGCCAAAGTGGAAAAGGGCAAGTCCAAG
AAACTGAAGAGTGTGAAAGAGCTGCTGGGGATCACCATCATGGAAAGAAGCAGCTT
CGAGAAGAATCCCATCGACTTTCTGGAAGCCAAGGGCTACAAAGAAGTGAAAAAGG
ACCTGATCATCAAGCTGCCTAAGTACTCCCTGTTCGAGCTGGAAAACGGCCGGAAG
AGAATGCTGGCCTCTGCCGGCGAACTGCAGAAGGGAAACGAACTGGCCCTGCCCTC
CAAATATGTGAACTTCCTGTACCTGGCCAGCCACTATGAGAAGCTGAAGGGCTCCCC
CGAGGATAATGAGCAGAAACAGCTGTTTGTGGAACAGCACAAGCACTACCTGGACG
AGATCATCGAGCAGATCAGCGAGTTCTCCAAGAGAGTGATCCTGGCCGACGCTAAT
CTGGACAAAGTGCTGTCCGCCTACAACAAGCACCGGGATAAGCCCATCAGAGAGCA
GGCCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGTGC
TGGACGCCACCCTGATCCACCAGAGCATCACCGGCCTGTACGAGACACGGATCGAC
CTGTCTCAGCTGGGAGGCGACAAGCGTCCTGCTGCTACTAAGAAAGCTGGTCAAGCT
AAGAAAAAGAAA
> hEMX1-HRTemplate-HindIII-NheI
(SEQ ID NO: 81)
GAATGCTGCCCTCAGACCCGCTTCCTCCCTGTCCTTGTCTGTCCAAGGAGA
ATGAGGTCTCACTGGTGGATTTCGGACTACCCTGAGGAGCTGGCACCTGAGGGACA
AGGCCCCCCACCTGCCCAGCTCCAGCCTCTGATGAGGGGTGGGAGAGAGCTACATG
AGGTTGCTAAGAAAGCCTCCCCTGAAGGAGACCACACAGTGTGTGAGGTTGGAGTC
TCTAGCAGCGGGTTCTGTGCCCCCAGGGATAGTCTGGCTGTCCAGGCACTGCTCTTG
ATATAAACACCACCTCCTAGTTATGAAACCATGCCCATTCTGCCTCTCTGTATGGAA
AAGAGCATGGGGCTGGCCCGTGGGGTGGTGTCCACTTTAGGCCCTGTGGGAGATCA
TGGGAACCCACGCAGTGGGTCATAGGCTCTCTCATTTACTACTCACATCCACTCTGT
GAAGAAGCGATTATGATCTCTCCTCTAGAAACTCGTAGAGTCCCATGTCTGCCGGCT
TCCAGAGCCTGCACTCCTCCACCTTGGCTTGGCTTTGCTGGGGCTAGAGGAGCTAGG
ATGCACAGCAGCTCTGTGACCCTTTGTTTGAGAGGAACAGGAAAACCACCCTTCTCT
CTGGCCCACTGTGTCCTCTTCCTGCCCTGCCATCCCCTTCTGTGAATGTTAGACCCAT
GGGAGCAGCTGGTCAGAGGGGACCCCGGCCTGGGGCCCCTAACCCTATGTAGCCTC
AGTCTTCCCATCAGGCTCTCAGCTCAGCCTGAGTGTTGAGGCCCCAGTGGCTGCTCT
GGGGGCCTCCTGAGTTTCTCATCTGTGCCCCTCCCTCCCTGGCCCAGGTGAAGGTGT
GGTTCCAGAACCGGAGGACAAAGTACAAACGGCAGAAGCTGGAGGAGGAAGGGCC
TGAGTCCGAGCAGAAGAAGAAGGGCTCCCATCACATCAACCGGTGGCGCATTGCCA
CGAAGCAGGCCAATGGGGAGGACATCGATGTCACCTCCAATGACaagcttgctagcGGTGG
GCAACCACAAACCCACGAGGGCAGAGTGCTGCTTGCTGCTGGCCAGGCCCCTGCGT
GGGCCCAAGCTGGACTCTGGCCACTCCCTGGCCAGGCTTTGGGGAGGCCTGGAGTC
ATGGCCCCACAGGGCTTGAAGCCCGGGGCCGCCATTGACAGAGGGACAAGCAATGG
GCTGGCTGAGGCCTGGGACCACTTGGCCTTCTCCTCGGAGAGCCTGCCTGCCTGGGC
GGGCCCGCCCGCCACCGCAGCCTCCCAGCTGCTCTCCGTGTCTCCAATCTCCCTTTTG
TTTTGATGCATTTCTGTTTTAATTTATTTTCCAGGCACCACTGTAGTTTAGTGATCCCC
AGTGTCCCCCTTCCCTATGGGAATAATAAAAGTCTCTCTCTTAATGACACGGGCATC
CAGCTCCAGCCCCAGAGCCTGGGGTGGTAGATTCCGGCTCTGAGGGCCAGTGGGGG
CTGGTAGAGCAAACGCGTTCAGGGCCTGGGAGCCTGGGGTGGGGTACTGGTGGAGG
GGGTCAAGGGTAATTCATTAACTCCTCTCTTTTGTTGGGGGACCCTGGTCTCTACCTC
CAGCTCCACAGCAGGAGAAACAGGCTAGACATAGGGAAGGGCCATCCTGTATCTTG
AGGGAGGACAGGCCCAGGTCTTTCTTAACGTATTGAGAGGTGGGAATCAGGCCCAG
GTAGTTCAATGGGAGAGGGAGAGTGCTTCCCTCTGCCTAGAGACTCTGGTGGCTTCT
CCAGTTGAGGAGAAACCAGAGGAAAGGGGAGGATTGGGGTCTGGGGGAGGGAACA
CCATTCACAAAGGCTGACGGTTCCAGTCCGAAGTCGTGGGCCCACCAGGATGCTCA
CCTGTCCTTGGAGAACCGCTGGGCAGGTTGAGACTGCAGAGACAGGGCTTAAGGCT
GAGCCTGCAACCAGTCCCCAGTGACTCAGGGCCTCCTCAGCCCAAGAAAGAGCAAC
GTGCCAGGGCCCGCTGAGCTCTTGTGTTCACCTG
> NLS-StCsn1-NLS
(SEQ ID NO: 82)
ATGAAAAGGCCGGCGGCCACGAAAAAGGCCGGCCAGGCAAAAAAGAAA
AAGTCCGACCTGGTACTTGGACTGGATATTGGTATCGGTTCGGTGGGAGTCGGAATC
CTCAACAAGGTCACGGGGGAGATCATTCACAAGAACTCGCGGATCTTCCCCGCAGC
TCAGGCTGAGAACAACTTGGTGCGGAGAACG
AATAGGCAGGGCAGGCGACTGGCGAGGAGGAAGAAACACAGGAGAGTC
CGATTGAACCGGCTGTTCGAGGAGTCCGGTTTGATCACCGACTTTACGAAAATCTCG
ATTAACCTTAATCCCTATCAGCTTCGGGTGAAAGGCCTGACAGACGAACTTTCGAAT
GAGGAACTTTTCATCGCGCTGAAAAACATGGTCAAGCACAGAGGGATTTCCTACCTC
GATGACGCCTCGGATGACGGAAATTCCTCAGTAGGAGATTATGCACAGATCGTGAA
AGAGAACTCAAAGCAACTGGAAACAAAGACACCGGGGCAGATCCAACTTGAAAGA
TACCAGACATACGGACAGCTCAGAGGAGATTTTACGGTGGAGAAGGACGGTAAAAA
GCACAGACTCATTAACGTATTTCCCACGTCGGCGTACAGATCCGAAGCGCTCCGCAT
CCTTCAGACTCAACAGGAGTTCAACCCGCAAATTACTGATGAGTTCATCAACCGCTA
TTTGGAAATCTTGACCGGAAAGCGCAAGTATTATCATGGGCCGGGTAATGAGAAAT
CCAGAACAGATTACGGCCGATACAGAACTTCGGGGGAAACCTTGGATAACATCTTT
GGTATTTTGATTGGAAAGTGCACCTTTTACCCGGACGAGTTTCGAGCGGCCAAGGCG
TCATACACAGCACAAGAGTTTAATCTCTTGAATGATTTGAACAACTTGACGGTCCCC
ACGGAGACAAAGAAGCTCTCCAAAGAGCAAAAGAACCAAATCATCAACTACGTCA
AGAACGAGAAGGCTATGGGGCCAGCGAAGCTGTTCAAGTATATCGCTAAACTTCTC
AGCTGTGATGTGGCGGACATCAAAGGGTACCGAATCGACAAGTCGGGAAAAGCGGA
AATTCACACGTTTGAAGCATATCGAAAGATGAAAACGTTGGAAACACTGGACATTG
AGCAGATGGACCGGGAAACGCTCGACAAACTGGCATACGTGCTCACGTTGAATACT
GAACGAGAGGGAATCCAAGAGGCCCTTGAACATGAGTTCGCCGATGGATCGTTCAG
CCAGAAGCAGGTCGACGAACTTGTGCAATTCCGCAAGGCGAATAGCTCCATCTTCG
GGAAGGGATGGCACAACTTTTCGGTCAAACTCATGATGGAGTTGATCCCAGAACTTT
ATGAGACTTCGGAGGAGCAAATGACGATCTTGACGCGCTTGGGGAAACAGAAAACG
ACAAGCTCATCGAACAAAACTAAGTACATTGATGAGAAATTGCTGACGGAAGAAAT
CTATAATCCGGTAGTAGCGAAATCGGTAAGACAAGCGATCAAAATCGTGAACGCGG
CGATCAAGGAATATGGTGACTTTGATAACATCGTAATTGAAATGGCTAGAGAGACG
AACGAAGATGACGAGAAAAAGGCAATCCAGAAGATCCAGAAGGCCAACAAGGATG
AAAAAGATGCAGCGATGCTTAAAGCGGCCAACCAATACAATGGAAAGGCGGAGCT
GCCCCATTCAGTGTTTCACGGTCATAAACAGTTGGCGACCAAGATCCGACTCTGGCA
TCAGCAGGGTGAGCGGTGTCTCTACACCGGAAAGACTATCTCCATCCATGACTTGAT
TAACAATTCGAACCAGTTTGAAGTGGATCATATTCTGCCCCTGTCAATCACCTTTGA
CGACTCGCTTGCGAACAAGGTGCTCGTGTACGCAACGGCAAATCAGGAGAAAGGCC
AGCGGACTCCGTATCAGGCGCTCGACTCAATGGACGATGCGTGGTCATTCCGGGAG
CTGAAGGCGTTCGTACGCGAGAGCAAGACACTGAGCAACAAAAAGAAAGAGTATCT
GCTGACAGAGGAGGACATCTCGAAATTCGATGTCAGGAAGAAGTTCATCGAGCGGA
ATCTTGTCGACACTCGCTACGCTTCCAGAGTAGTACTGAACGCGCTCCAGGAACACT
TTAGAGCGCACAAAATTGACACGAAGGTGTCAGTGGTGAGAGGGCAGTTCACATCC
CAACTCCGCCGACATTGGGGCATCGAAAAGACGCGGGACACATATCACCATCATGC
GGTGGACGCGCTGATTATTGCCGCTTCGTCCCAGTTGAATCTCTGGAAAAAGCAGAA
GAACACGCTGGTGTCGTATTCGGAGGATCAGCTTTTGGACATCGAAACCGGGGAGC
TGATTTCCGACGATGAATACAAAGAATCGGTGTTTAAGGCACCATATCAGCATTTCG
TGGACACGCTGAAGAGCAAAGAGTTTGAGGACAGCATCCTCTTTTCGTACCAAGTG
GACTCGAAGTTTAATCGCAAGATTTCAGACGCCACAATCTACGCGACGAGGCAGGC
GAAGGTGGGCAAAGATAAAGCAGATGAAACCTACGTCCTTGGTAAAATCAAGGACA
TCTACACTCAGGACGGGTACGATGCGTTCATGAAAATCTACAAGAAGGATAAGTCG
AAGTTTCTCATGTACCGCCACGATCCACAGACTTTCGAAAAAGTCATTGAGCCTATT
TTGGAGAACTACCCTAACAAGCAAATCAACGAGAAAGGGAAAGAAGTCCCGTGCAA
CCCCTTTCTGAAGTACAAGGAAGAGCACGGTTATATCCGCAAATACTCGAAGAAAG
GAAATGGGCCTGAGATTAAGTCGCTTAAGTATTACGACTCAAAGTTGGGTAACCAC
ATCGACATTACCCCGAAAGACTCCAACAACAAAGTCGTGTTGCAGTCCGTCTCGCCC
TGGCGAGCAGATGTGTATTTTAATAAGACGACCGGCAAATATGAGATCCTTGGACTC
AAATACGCAGACCTTCAATTCGAAAAGGGGACGGGCACTTATAAGATTTCACAAGA
GAAGTACAACGACATCAAGAAAAAGGAAGGGGTCGATTCAGATTCGGAGTTCAAAT
TCACCCTCTACAAAAACGACCTCCTGCTTGTGAAGGACACAGAAACGAAGGAGCAG
CAGCTCTTTCGGTTCCTCTCACGCACGATGCCCAAACAAAAACATTACGTCGAACTT
AAACCTTACGATAAGCAAAAGTTTGAAGGGGGAGAGGCACTGATCAAAGTATTGGG
TAACGTAGCCAATAGCGGACAGTGTAAGAAAGGGCTGGGAAAGTCCAATATCTCGA
TCTATAAAGTACGAACAGATGTATTGGGAAACCAGCATATCATCAAAAATGAGGGG
GATAAACCCAAACTCGATTTCAAGCGTCCTGCTGCTACTAAGAAAGCTGGTCAAGCT
AAGAAAAAGAAATAA
> U6-St_tracrRNA(7-97)
(SEQ ID NO: 83)
GAGGGCCTATTTCCCATGATTCCTTCATATTTGCATATACGATACAAGGCT
GTTAGAGAGATAATTGGAATTAATTTGACTGTAAACACAAAGATATTAGTACAAAA
TACGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTTAT
ATATCTTGTGGAAAGGACGAAACACCGTTACTTAAATCTTGCAGAAGCTACAAAGA
TAAGGCTTCATGCCGAAATCAACACCCTGTCATTTTATGGCAGGGTGTTTTCGTTATT
TAA
>EMX1_TALEN_Left
(SEQ ID NO: 84)
ATGGACTATAAGGACCACGACGGAGACTACAAGGATCATGATATTGATTA
CAAAGACGATGACGATAAGATGGCCCCAAAGAAGAAGCGGAAGGTCGGTATCCAC
GGAGTCCCAGCAGCCGTAGATTTGAGAACTTTGGGATATTCACAGCAGCAGCAGGA
AAAGATCAAGCCCAAAGTGAGGTCGACAGTCGCGCAGCATCACGAAGCGCTGGTGG
GTCATGGGTTTACACATGCCCACATCGTAGCCTTGTCGCAGCACCCTGCAGCCCTTG
GCACGGTCGCCGTCAAGTACCAGGACATGATTGCGGCGTTGCCGGAAGCCACACAT
GAGGCGATCGTCGGTGTGGGGAAACAGTGGAGCGGAGCCCGAGCGCTTGAGGCCCT
GTTGACGGTCGCGGGAGAGCTGAGAGGGCCTCCCCTTCAGCTGGACACGGGCCAGT
TGCTGAAGATCGCGAAGCGGGGAGGAGTCACGGCGGTCGAGGCGGTGCACGCGTGG
CGCAATGCGCTCACGGGAGCACCCCTCAACCTGACCCCAGAGCAGGTCGTGGCAAT
TGCGAGCAACCACGGGGGAAAGCAGGCACTCGAAACCGTCCAGAGGTTGCTGCCTG
TGCTGTGCCAAGCGCACGGACTTACGCCAGAGCAGGTCGTGGCAATTGCGAGCAAC
CACGGGGGAAAGCAGGCACTCGAAACCGTCCAGAGGTTGCTGCCTGTGCTGTGCCA
AGCGCACGGACTAACCCCAGAGCAGGTCGTGGCAATTGCGAGCAACATCGGGGGAA
AGCAGGCACTCGAAACCGTCCAGAGGTTGCTGCCTGTGCTGTGCCAAGCGCACGGG
TTGACCCCAGAGCAGGTCGTGGCAATTGCGAGCAACCACGGGGGAAAGCAGGCACT
CGAAACCGTCCAGAGGTTGCTGCCTGTGCTGTGCCAAGCGCACGGCCTGACCCCAG
AGCAGGTCGTGGCAATTGCGAGCAACCACGGGGGAAAGCAGGCACTCGAAACCGTC
CAGAGGTTGCTGCCTGTGCTGTGCCAAGCGCACGGACTGACACCAGAGCAGGTCGT
GGCAATTGCGAGCAACATCGGGGGAAAGCAGGCACTCGAAACCGTCCAGAGGTTGC
TGCCTGTGCTGTGCCAAGCGCACGGACTTACACCCGAACAAGTCGTGGCAATTGCG
AGCAACCACGGGGGAAAGCAGGCACTCGAAACCGTCCAGAGGTTGCTGCCTGTGCT
GTGCCAAGCGCACGGACTTACGCCAGAGCAGGTCGTGGCAATTGCGAGCAACCACG
GGGGAAAGCAGGCACTCGAAACCGTCCAGAGGTTGCTGCCTGTGCTGTGCCAAGCG
CACGGACTAACCCCAGAGCAGGTCGTGGCAATTGCGAGCAACATCGGGGGAAAGCA
GGCACTCGAAACCGTCCAGAGGTTGCTGCCTGTGCTGTGCCAAGCGCACGGGTTGA
CCCCAGAGCAGGTCGTGGCAATTGCGAGCAACATCGGGGGAAAGCAGGCACTCGAA
ACCGTCCAGAGGTTGCTGCCTGTGCTGTGCCAAGCGCACGGCCTGACCCCAGAGCA
GGTCGTGGCAATTGCGAGCAACCACGGGGGAAAGCAGGCACTCGAAACCGTCCAGA
GGTTGCTGCCTGTGCTGTGCCAAGCGCACGGACTGACACCAGAGCAGGTCGTGGCA
ATTGCGAGCAACCACGGGGGAAAGCAGGCACTCGAAACCGTCCAGAGGTTGCTGCC
TGTGCTGTGCCAAGCGCACGGACTCACGCCTGAGCAGGTAGTGGCTATTGCATCCAA
CAACGGGGGCAGACCCGCACTGGAGTCAATCGTGGCCCAGCTTTCGAGGCCGGACC
CCGCGCTGGCCGCACTCACTAATGATCATCTTGTAGCGCTGGCCTGCCTCGGCGGAC
GACCCGCCTTGGATGCGGTGAAGAAGGGGCTCCCGCACGCGCCTGCATTGATTAAG
CGGACCAACAGAAGGATTCCCGAGAGGACATCACATCGAGTGGCAGGTTCCCAACT
CGTGAAGAGTGAACTTGAGGAGAAAAAGTCGGAGCTGCGGCACAAATTGAAATACG
TACCGCATGAATACATCGAACTTATCGAAATTGCTAGGAACTCGACTCAAGACAGA
ATCCTTGAGATGAAGGTAATGGAGTTCTTTATGAAGGTTTATGGATACCGAGGGAAG
CATCTCGGTGGATCACGAAAACCCGACGGAGCAATCTATACGGTGGGGAGCCCGAT
TGATTACGGAGTGATCGTCGACACGAAAGCCTACAGCGGTGGGTACAATCTTCCCAT
CGGGCAGGCAGATGAGATGCAACGTTATGTCGAAGAAAATCAGACCAGGAACAAA
CACATCAATCCAAATGAGTGGTGGAAAGTGTATCCTTCATCAGTGACCGAGTTTAAG
TTTTTGTTTGTCTCTGGGCATTTCAAAGGCAACTATAAGGCCCAGCTCACACGGTTG
AATCACATTACGAACTGCAATGGTGCGGTTTTGTCCGTAGAGGAACTGCTCATTGGT
GGAGAAATGATCAAAGCGGGAACTCTGACACTGGAAGAAGTCAGACGCAAGTTTAA
CAATGGCGAGATCAATTTCCGCTCA
>EMX1_TALEN_Right
(SEQ ID NO: 85)
ATGGACTATAAGGACCACGACGGAGACTACAAGGATCATGATATTGATTA
CAAAGACGATGACGATAAGATGGCCCCAAAGAAGAAGCGGAAGGTCGGTATCCAC
GGAGTCCCAGCAGCCGTAGATTTGAGAACTTTGGGATATTCACAGCAGCAGCAGGA
AAAGATCAAGCCCAAAGTGAGGTCGACAGTCGCGCAGCATCACGAAGCGCTGGTGG
GTCATGGGTTTACACATGCCCACATCGTAGCCTTGTCGCAGCACCCTGCAGCCCTTG
GCACGGTCGCCGTCAAGTACCAGGACATGATTGCGGCGTTGCCGGAAGCCACACAT
GAGGCGATCGTCGGTGTGGGGAAACAGTGGAGCGGAGCCCGAGCGCTTGAGGCCCT
GTTGACGGTCGCGGGAGAGCTGAGAGGGCCTCCCCTTCAGCTGGACACGGGCCAGT
TGCTGAAGATCGCGAAGCGGGGAGGAGTCACGGCGGTCGAGGCGGTGCACGCGTGG
CGCAATGCGCTCACGGGAGCACCCCTCAACCTGACCCCAGAGCAGGTCGTGGCAAT
TGCGAGCAACCACGGGGGAAAGCAGGCACTCGAAACCGTCCAGAGGTTGCTGCCTG
TGCTGTGCCAAGCGCACGGACTTACGCCAGAGCAGGTCGTGGCAATTGCGAGCAAC
CACGGGGGAAAGCAGGCACTCGAAACCGTCCAGAGGTTGCTGCCTGTGCTGTGCCA
AGCGCACGGACTAACCCCAGAGCAGGTCGTGGCAATTGCGAGCAACCACGGGGGA
AAGCAGGCACTCGAAACCGTCCAGAGGTTGCTGCCTGTGCTGTGCCAAGCGCACGG
GTTGACCCCAGAGCAGGTCGTGGCAATTGCGAGCAACATCGGGGGAAAGCAGGCAC
TCGAAACCGTCCAGAGGTTGCTGCCTGTGCTGTGCCAAGCGCACGGCCTGACCCCAG
AGCAGGTCGTGGCAATTGCGAGCAACCACGGGGGAAAGCAGGCACTCGAAACCGTC
CAGAGGTTGCTGCCTGTGCTGTGCCAAGCGCACGGACTGACACCAGAGCAGGTCGT
GGCAATTGCGAGCCATGACGGGGGAAAGCAGGCACTCGAAACCGTCCAGAGGTTGC
TGCCTGTGCTGTGCCAAGCGCACGGACTTACACCCGAACAAGTCGTGGCAATTGCG
AGCCATGACGGGGGAAAGCAGGCACTCGAAACCGTCCAGAGGTTGCTGCCTGTGCT
GTGCCAAGCGCACGGACTTACGCCAGAGCAGGTCGTGGCAATTGCGAGCCATGACG
GGGGAAAGCAGGCACTCGAAACCGTCCAGAGGTTGCTGCCTGTGCTGTGCCAAGCG
CACGGACTAACCCCAGAGCAGGTCGTGGCAATTGCGAGCAACGGAGGGGGAAAGC
AGGCACTCGAAACCGTCCAGAGGTTGCTGCCTGTGCTGTGCCAAGCGCACGGGTTG
ACCCCAGAGCAGGTCGTGGCAATTGCGAGCAACGGAGGGGGAAAGCAGGCACTCG
AAACCGTCCAGAGGTTGCTGCCTGTGCTGTGCCAAGCGCACGGCCTGACCCCAGAG
CAGGTCGTGGCAATTGCGAGCCATGACGGGGGAAAGCAGGCACTCGAAACCGTCCA
GAGGTTGCTGCCTGTGCTGTGCCAAGCGCACGGACTGACACCAGAGCAGGTCGTGG
CAATTGCGAGCAACGGAGGGGGAAAGCAGGCACTCGAAACCGTCCAGAGGTTGCTG
CCTGTGCTGTGCCAAGCGCACGGACTCACGCCTGAGCAGGTAGTGGCTATTGCATCC
AACGGAGGGGGCAGACCCGCACTGGAGTCAATCGTGGCCCAGCTTTCGAGGCCGGA
CCCCGCGCTGGCCGCACTCACTAATGATCATCTTGTAGCGCTGGCCTGCCTCGGCGG
ACGACCCGCCTTGGATGCGGTGAAGAAGGGGCTCCCGCACGCGCCTGCATTGATTA
AGCGGACCAACAGAAGGATTCCCGAGAGGAATCACATCGAGTGGCAGGTTCCCAAC
TCGTGAAGAGTGAACTTGAGGAGAAAAAGTCGGAGCTGCGGCACAAATTGAAATAC
GTACCGCATGAATACATCGAACTTATCGAAATTGCTAGGAACTCGACTCAAGACAG
AATCCTTGAGATGAAGGTAATGGAGTTCTTTATGAAGGTTTATGGATACCGAGGGAA
GCATCTCGGTGGATCACGAAAACCCGACGGAGCAATCTATACGGTGGGGAGCCCGA
TTGATTACGGAGTGATCGTCGACACGAAAGCCTACAGCGGTGGGTACAATCTTCCCA
TCGGGCAGGCAGATGAGATGCAACGTTATGTCGAAGAAAATCAGACCAGGAACAAA
CACATCAATCCAAATGAGTGGTGGAAAGTGTATCCTTCATCAGTGACCGAGTTTAAG
TTTTTGTTTGTCTCTGGGCATTTCAAAGGCAACTATAAGGCCCAGCTCACACGGTTG
AATCACATTACGAACTGCAATGGTGCGGTTTTGTCCGTAGAGGAACTGCTCATTGGT
GGAGAAATGATCAAAGCGGGAACTCTGACACTGGAAGAAGTCAGACGCAAGTTTAA
CAATGGCGAGATCAATTTCCGCTCA

### Example 9

### Cloning (Construction) of AAV Constructs

Construction of AAV-Promoter-TALE-Effector Backbone.

For construction of AAV-promoter-TALE-effector a backbone was cloned by standard subcloning methods. Specifically, the vector contained an antibiotics resistance gene, such as ampicillin resistance and two AAV inverted terminal repeats (itr's) flanking the promoter-TALE-effector insert (sequences, see below). The promoter (hSyn), the effector domain (VP64, SID4X or CIB1 in this example)/the N- and C-terminal portion of the TALE gene containing a spacer with two typeIIS restriction sites (BsaI in this instance) were subcloned into this vector. To achieve subcloning, each DNA component was amplified using polymerase-chain reaction and then digested with specific restriction enzymes to create matching DNA sticky ends. The vector was similarly digested with DNA restriction enzymes. All DNA fragments were subsequently allowed to anneal at matching ends and fused together using a ligase enzyme.

Assembly of Individual TALEs into AAV-Promoter-TALE-Effector Backbone.

For incorporating different TALE monomer sequences into the AAV-promoter-TALE-effector backbone described above, a strategy based on restriction of individual monomers with type IIS restriction enzymes and ligation of their unique overhangs to form an assembly of 12 to 16 monomers to form the final TALE and ligate it into the AAV-promoter-TALE-effector backbone by using the type IIS sites present in the spacer between the N- and C-term (termed golden gate assembly). This method of TALE monomer assembly has previously been described by us (NE Sanjana, L Cong, Y Zhou, M M Cunniff, G Feng & F Zhang A transcription activator-like effector toolbox for genome engineering Nature Protocols 7, 171-192 (2012) doi:10.1038/nprot.2011.431)

By using the general cloning strategy outlined above, AAV vectors containing different promoters, effector domains and TALE monomer sequences can be easily constructed.

Nucleotide Sequences:

Left AAV ITR

(SEQ ID NO: 86)

cctgcaggcagctgcgcgctcgctcgctcactgaggccgcccgggcaaag
cccgggcgtcgggcgacctttggtcgcccggcctcagtgagcgagcgagc
gcgcagagagggagtggccaactccatcactaggggttcct
Right AAV ITR

(SEQ ID NO: 87)

Aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagctgcctgcagg
hSyn promoter

(SEQ ID NO: 88)

gtgtctagactgcagagggccctgcgtatgagtgcaagtgggttttagga
ccaggatgaggcggggtgggggtgcctacctgacgaccgaccccgaccca
ctggacaagcacccaacccccattccccaaattgcgcatcccctatcaga
gagggggaggggaaacaggatgcggcgaggcgcgtgcgcactgccagctt
cagcaccgcggacagtgccttcgcccccgcctggcggcgcgcgccaccgc
cgcctcagcactgaaggcgcgctgacgtcactcgccggtcccccgcaaac
tccccttcccggccaccttggtcgcgtccgcgccgccgccggcccagccg
gaccgcaccacgcgaggcgcgagataggggggcacgggcgcgaccatctg
cgctgcggcgccggcgactcagcgctgcctcagtctgcggtgggcagcgg
aggagtcgtgtcgtgcctgagagcgcagtcgagaa
TALE N-term (+136 AA truncation)

(SEQ ID NO: 89)

GTAGATTTGAGAACTTTGGGATATTCACAGCAGCAGCAGGAAAAGATCAA
GCCCAAAGTGAGGTCGACAGTCGCGCAGCATCACGAAGCGCTGGTGGGTC
ATGGGTTTACACATGCCCACATCGTAGCCTTGTCGCAGCACCCTGCAGCC
CTTGGCACGGTCGCCGTCAAGTACCAGGACATGATTGCGGCGTTGCCGGA
AGCCACACATGAGGCGATCGTCGGTGTGGGGAAACAGTGGAGCGGAGCCC
GAGCGCTTGAGGCCCTGTTGACGGTCGCGGGAGAGCTGAGAGGGCCTCCC
CTTCAGCTGGACACGGGCCAGTTGCTGAAGATCGCGAAGCGGGGAGGAGT
CACGGCGGTCGAGGCGGTGCACGCGTGGCGCAATGCGCTCACGGGAGCAC
CCCTCAAC
TALE C-term (+63 AA truncation)

(SEQ ID NO: 90)

CGGACCCCGCGCTGGCCGCACTCACTAATGATCATCTTGTAGCGCTGGCC
TGCCTCGGCGGACGACCCGCCTTGGATGCGGTGAAGAAGGGGCTCCCGCA
CGCGCCTGCATTGATTAAGCGGACCAACAGAAGGATTCCCGAGAGGACAT
CACATCGAGTGG
CA
Ampicillin resistance gene

(SEQ ID NO: 91)

atgagtattcaacatttccgtgtcgcccttattcccttttttgcggcatt
ttgccttcctgtttttgctcacccagaaacgctggtgaaagtaaaagatg
ctgaagatcagttgggtgcacgagtgggttacatcgaactggatctcaac
agcggtaagatccttgagagttttcgccccgaagaacgttttccaatgat
gagcacttttaaagttctgctatgtggcgcggtattatcccgtattgacg
ccgggcaagagcaactcggtcgccgcatacactattctcagaatgacttg
gttgagtactcaccagtcacagaaaagcatcttacggatggcatgacagt
aagagaattatgcagtgctgccataaccatgagtgataacactgcggcca
acttacttctgacaacgatcggaggaccgaaggagctaaccgcttttttg
cacaacatgggggatcatgtaactcgccttgatcgttgggaaccggagct
gaatgaagccataccaaacgacgagcgtgacaccacgatgcctgtagcaa
tggcaacaacgttgcgcaaactattaactggcgaactacttactctagct
tcccggcaacaattaatagactggatggaggcggataaagttgcaggacc
acttctgcgctcggcccttccggctggctggtttattgctgataaatctg
gagccggtgagcgtgggtctcgcggtatcattgcagcactggggccagat
ggtaagccctcccgtatcgtagttatctacacgacggggagtcaggcaac
tatggatgaacgaaatagacagatcgctgagataggtgcctcactgatta
agcattgg

### Example 10

### Optical Control of Endogenous Mammalian Transcription

The ability to directly modulate transcription of the endogenous mammalian genome is critical for elucidating normal gene function and disease mechanisms. Here, Applicants describe the development of Light-Inducible Transcriptional Effectors (LITEs), a two-hybrid system integrating the customizable TALE DNA-binding domain with the light-sensitive cryptochrome 2 protein and its interacting partner CIB1 from Arabidopsis thaliana. LITEs can be activated within minutes, mediating reversible bidirectional regulation of endogenous mammalian gene expression as well as targeted epigenetic chromatin modifications. Applicants have applied this system in primary mouse neurons, as well as in the brain of awake, behaving mice in vivo. The LITE system establishes a novel mode of optogenetic control of endogenous cellular processes and enables direct testing of the causal roles of genetic and epigenetic regulation.

The dynamic nature of gene expression enables cellular programming, homeostasis, and environmental adaptation in living systems. Dissecting the contributions of genes to cellular and organismic function therefore requires an approach that enables spatially and temporally controlled modulation of gene expression. Microbial and plant-derived light-sensitive proteins have been engineered as optogenetic actuators, enabling the use of light—which provides high spatiotemporal resolution—to control many cellular functions (Deisseroth, K. Optogenetics. Nature methods 8, 26-29, doi:10.1038/nmeth.f.324 (2011); Zhang, F. et al. The microbial opsin family of optogenetic tools. Cell 147, 1446-1457, doi:10.1016/j.cell.2011.12.004 (2011); Levskaya, A., Weiner, O. D., Lim, W. A. & Voigt, C. A. Spatiotemporal control of cell signalling using a light-switchable protein interaction. Nature 461, 997-1001, doi:10.1038/nature08446 (2009); Yazawa, M., Sadaghiani, A. M., Hsueh, B. & Dolmetsch, R. E. Induction of protein-protein interactions in live cells using light. Nature biotechnology 27, 941-945, doi:10.1038/nbt.1569 (2009); Strickland, D. et al. TULIPs: tunable, light-controlled interacting protein tags for cell biology. Nature methods 9, 379-384, doi:10.1038/nmeth.1904 (2012); Kennedy, M. J. et al. Rapid blue-light-mediated induction of protein interactions in living cells. Nature methods 7, 973-975, doi:10.1038/nmeth.1524 (2010); Shimizu-Sato, S., Huq, E., Tepperman, J. M. & Quail, P. H. A light-switchable gene promoter system. Nature biotechnology 20, 1041-1044, doi:10.1038/nbt734 (2002); Ye, H., Daoud-El Baba, M., Peng, R. W. & Fussenegger, M. A synthetic optogenetic transcription device enhances blood-glucose homeostasis in mice. Science 332, 1565-1568, doi:10.1126/science.1203535 (2011); Polstein, L. R. & Gersbach, C. A. Light-inducible spatiotemporal control of gene activation by customizable zinc finger transcription factors. Journal of the American Chemical Society 134, 16480-16483, doi:10.1021/ja3065667 (2012); Bugaj, L. J., Choksi, A. T., Mesuda, C. K., Kane, R. S. & Schaffer, D. V. Optogenetic protein clustering and signaling activation in mammalian cells. Nature methods (2013) and Zhang, F. et al. Multimodal fast optical interrogation of neural circuitry. Nature 446, 633-639, doi:10.1038/nature05744 (2007)). However, versatile and robust technologies to directly modulate endogenous transcriptional regulation using light remain elusive.

Here, Applicants report the development of Light-Inducible Transcriptional Effectors (LITEs), a modular optogenetic system that enables spatiotemporally precise control of endogenous genetic and epigenetic processes in mammalian cells. LITEs combine the programmable DNA-binding domain of transcription activator-like effectors (TALEs) (Boch, J. et al. Breaking the code of DNA binding specificity of TAL-type III effectors. Science 326, 1509-1512, doi:10.1126/science.1178811 (2009) and Moscou, M. J. & Bogdanove, A. J. A simple cipher governs DNA recognition by TAL effectors. Science 326, 1501, doi:10.1126/science.1178817 (2009)) from Xanthomonas sp. with the light-inducible heterodimeric proteins cryptochrome 2 (CRY2) and CIB1 from Arabidopsis thaliana (Kennedy, M. J. et al. Rapid blue-light-mediated induction of protein interactions in living cells. Nature methods 7, 973-975, doi:10.1038/nmeth.1524 (2010) and Liu, H. et al. Photoexcited CRY2 interacts with CIB1 to regulate transcription and floral initiation in Arabidopsis. Science 322, 1535-1539, doi:10.1126/science.1163927 (2008)). They do not require the introduction of heterologous genetic elements, do not depend on exogenous chemical co-factors, and exhibit fast and reversible dimerization kinetics (Levskaya, A., Weiner, O. D., Lim, W. A. & Voigt, C. A. Spatiotemporal control of cell signalling using a light-switchable protein interaction. Nature 461, 997-1001, doi:10.1038/nature08446 (2009); Yazawa, M., Sadaghiani, A. M., Hsueh, B. & Dolmetsch, R. E. Induction of protein-protein interactions in live cells using light. Nature biotechnology 27, 941-945, doi:10.1038/nbt.1569 (2009), Kennedy, M. J. et al. Rapid blue-light-mediated induction of protein interactions in living cells. Nature methods 7, 973-975, doi:10.1038/nmeth.1524 (2010); Shimizu-Sato, S., Huq, E., Tepperman, J. M. & Quail, P. H. A light-switchable gene promoter system. Nature biotechnology 20, 1041-1044, doi:10.1038/nbt734 (2002) and Liu, H. et al. Photoexcited CRY2 interacts with CIB1 to regulate transcription and floral initiation in Arabidopsis. Science 322, 1535-1539, doi:10.1126/science. 1163927 (2008)). Like other optogenetic tools, LITEs can be packaged into viral vectors and genetically targeted to probe specific cell populations. Applicants demonstrate the application of this system in primary neurons as well as in the mouse brain in vivo.

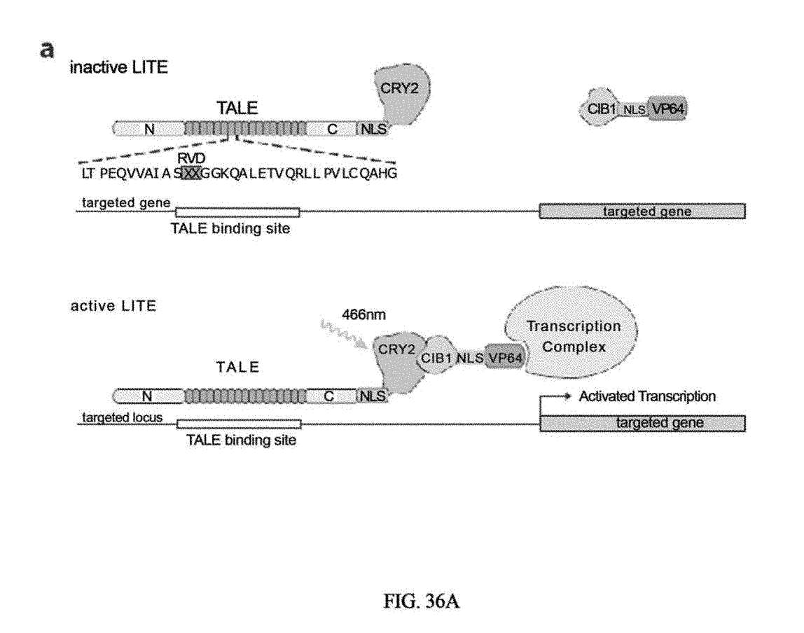

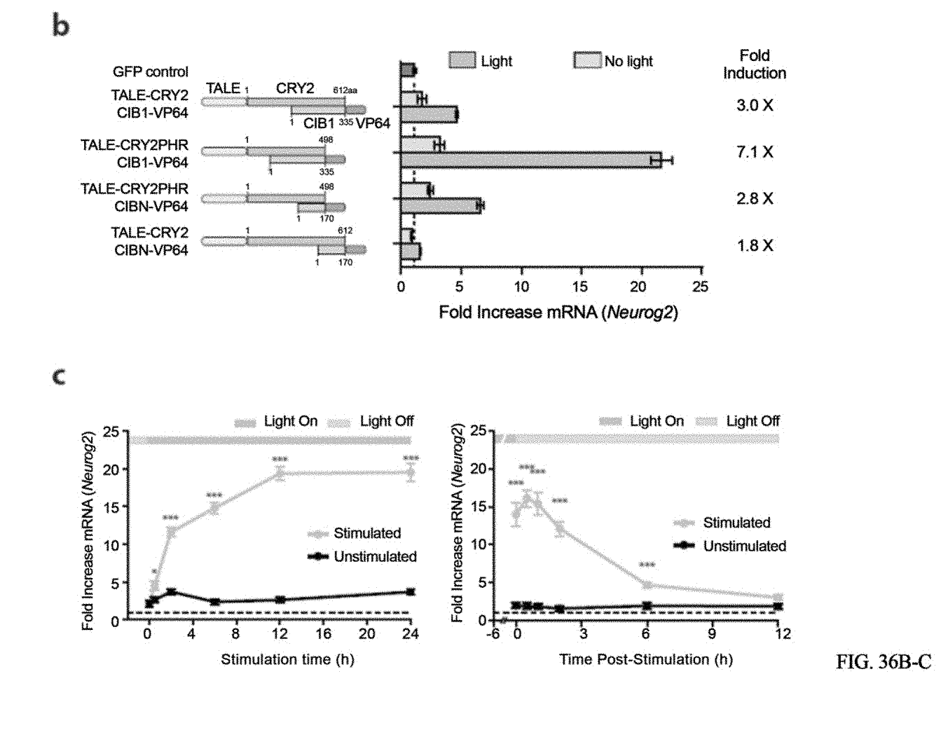

The LITE system contains two independent components (FIG. 36A): The first component is the genomic anchor and consists of a customized TALE DNA-binding domain fused to the light-sensitive CRY2 protein (TALE-CRY2). The second component consists of CIB1 fused to the desired transcriptional effector domain (CIB1-effector). To ensure effective nuclear targeting, Applicants attached a nuclear localization signal (NLS) to both modules. In the absence of light (inactive state), TALE-CRY2 binds the promoter region of the target gene while CIB1-effector remains free within the nuclear compartment. Illumination with blue light (peak ˜450 nm) triggers a conformational change in CRY2 and subsequently recruits CIB1-effector (VP64 shown in FIG. 36A) to the target locus to mediate transcriptional modulation. This modular design allows each LITE component to be independently engineered. For example, the same genomic anchor can be combined with activating or repressing effectors (Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proceedings of the National Academy of Sciences of the United States of America 95, 14628-14633 (1998) and Cong, L., Zhou, R., Kuo, Y.-c., Cunniff, M. & Zhang, F. Comprehensive interrogation of natural TALE DNA-binding modules and transcriptional repressor domains. Nat Commun 3, 968) to exert positive and negative transcriptional control over the same endogenous genomic locus.

In order to identify the most effective LITE architecture, Applicants fused TALE and the transcriptional activator VP64 (Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proceedings of the National Academy of Sciences of the United States of America 95, 14628-14633 (1998); Zhang, F. et al. Efficient construction of sequence-specific TAL effectors for modulating mammalian transcription. Nat Biotechnol 29, 149-153, doi:10.1038/nbt. 1775 (2011); Miller, J. C. et al. A TALE nuclease architecture for efficient genome editing. Nature biotechnology 29, 143-148, doi:10.1038/nbt.1755 (2011) and Hsu, P. D. & Zhang, F. Dissecting neural function using targeted genome engineering technologies. ACS chemical neuroscience 3, 603-610, doi:10.1021/cn300089k (2012).) to different truncations (Kennedy, M. J. et al. Rapid blue-light-mediated induction of protein interactions in living cells. Nature methods 7, 973-975, doi:10.1038/nmeth.1524 (2010)) of CRY2 and CIB1, respectively, and assessed the efficacy of each design by measuring blue light illumination induced transcriptional changes of the neural lineage-specifying transcription factor neurogenin 2 (Neurog2) (FIG. 36B). Applicants evaluated full-length CRY2 as well as a truncation consisting of the photolyase homology region alone (CRY2PHR, amino acids 1-498) (Kennedy, M. J. et al. Rapid blue-light-mediated induction of protein interactions in living cells. Nature methods 7, 973-975, doi:10.1038/nmeth.1524 (2010)). For CIB1, Applicants tested the full-length protein as well as an N-terminal domain-only fragment (CIBN, amino acids 1-170) (Kennedy, M. J. et al. Rapid blue-light-mediated induction of protein interactions in living cells. Nature methods 7, 973-975, doi:10.1038/nmeth.1524 (2010)). 3 out of 4 initial LITE pairings produced significant light-induced Neurog2 mRNA upregulation in Neuro 2a cells (p<0.001, FIG. 36B). Of these, TALE-CRY2PHR::CIB1-VP64 yielded the highest absolute light-mediated mRNA increase when normalized to either GFP-only control or unstimulated LITE samples (FIG. 36B), and was therefore applied in subsequent experiments.

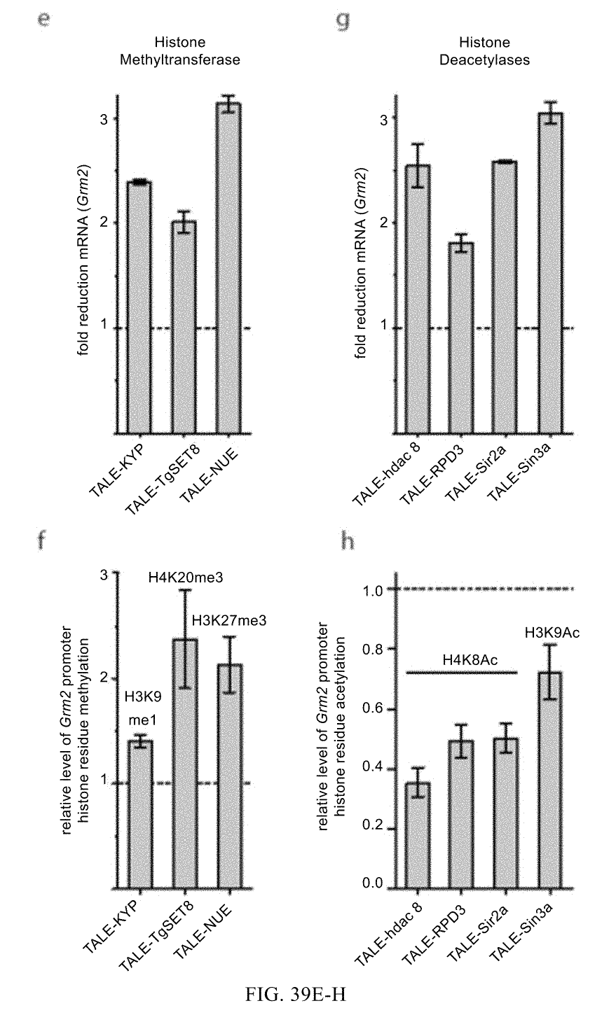

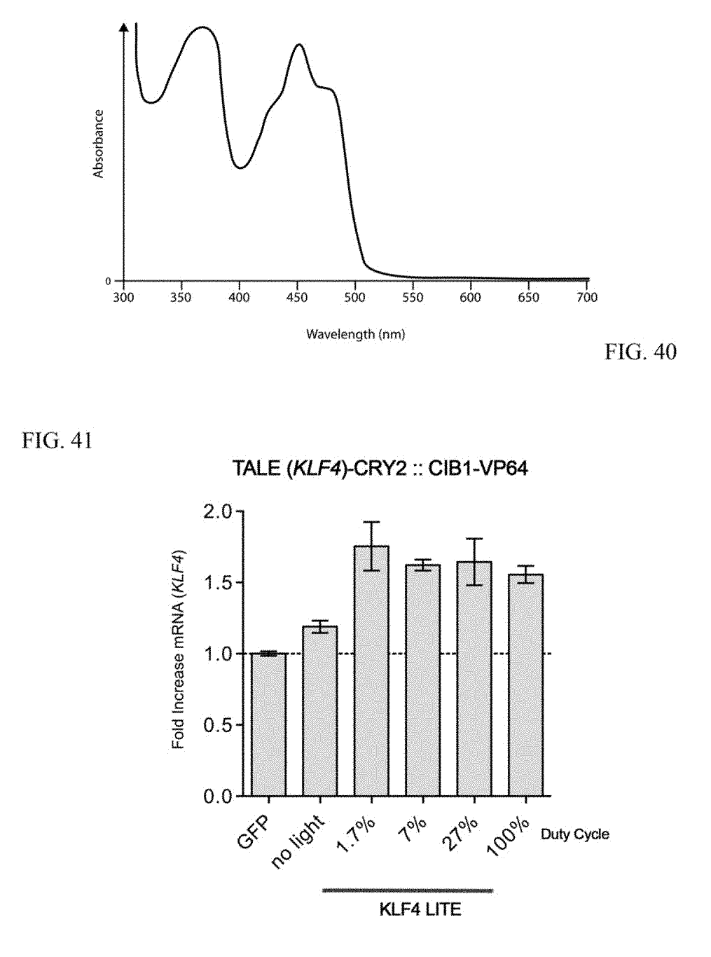

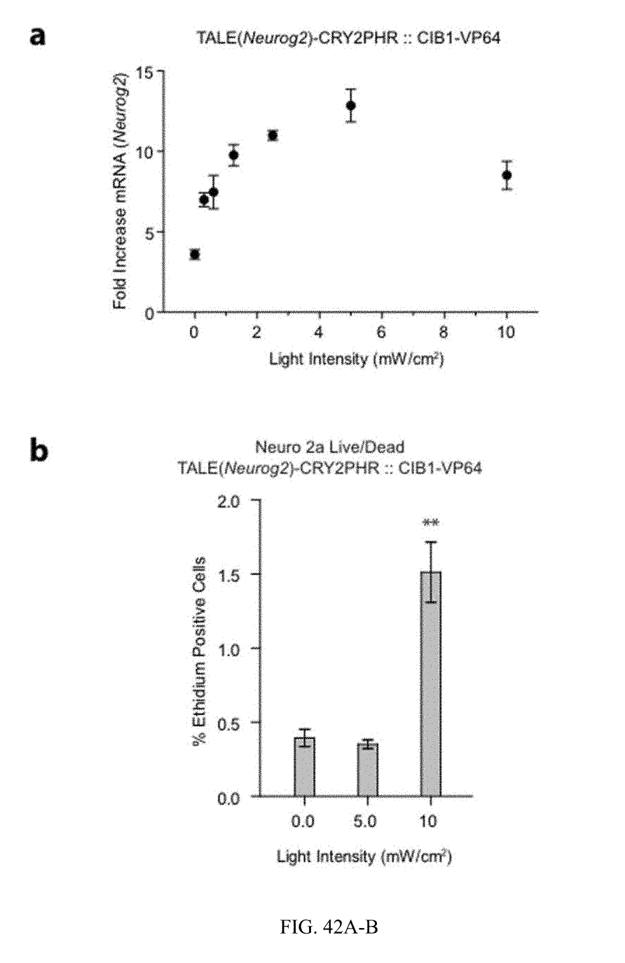

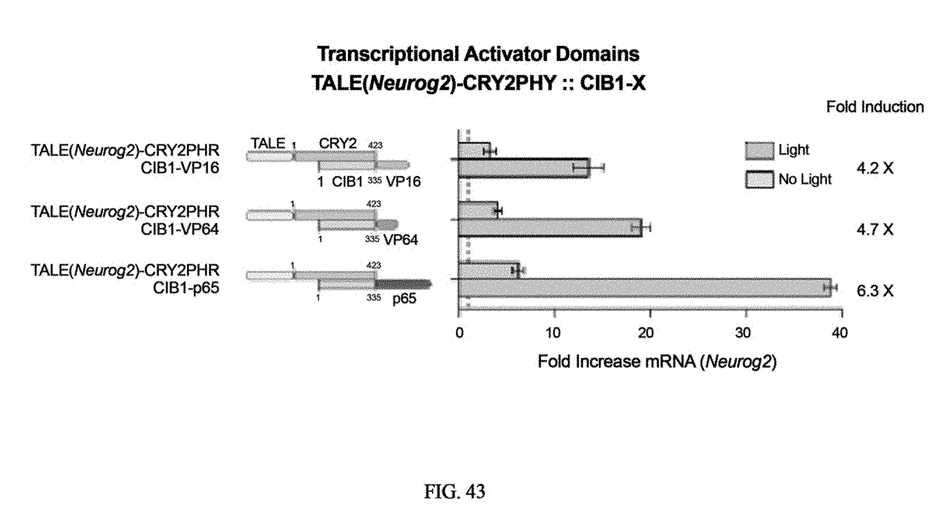

Having established an effective LITE architecture, Applicants systematically optimized light stimulation parameters, including wavelength (FIG. 40), duty cycle (FIG. 41), and light intensity (FIG. 42 and Example 11) (Banerjee, R. et al. The signaling state of Arabidopsis cryptochrome 2 contains flavin semiquinone. The Journal of biological chemistry 282, 14916-14922, doi:10.1074/jbc.M700616200 (2007)). Applicants also compared the activation domains VP16 and p65 in addition to VP64 to test the modularity of the LITE CIB1-effector component. All three domains produced a significant light-dependent Neurog2 mRNA upregulation (p<0.001, FIG. 43). Applicants selected VP64 for subsequent experiments due to its lower basal activity in the absence of light-stimulation.

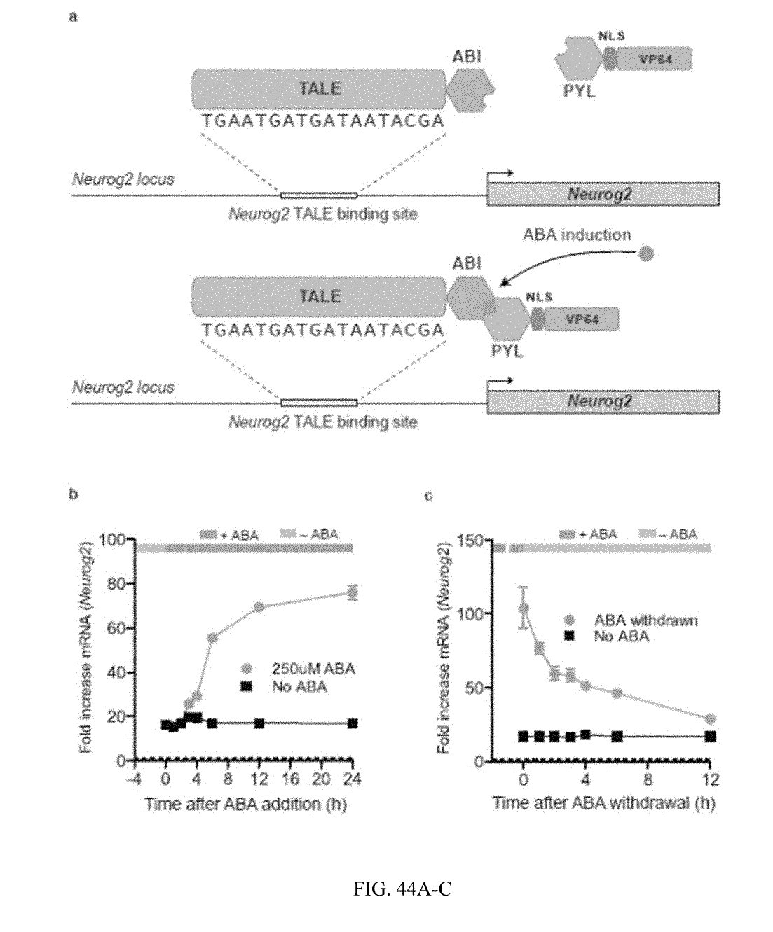

Manipulation of endogenous gene expression presents various challenges, as the rate of expression depends on many factors, including regulatory elements, mRNA processing, and transcript stability (Moore, M. J. & Proudfoot, N. J. Pre-mRNA processing reaches back to transcription and ahead to translation. Cell 136, 688-700, doi:10.1016/j.cell.2009.02.001 (2009) and Proudfoot, N. J., Furger, A. & Dye, M. J. Integrating mRNA processing with transcription. Cell 108, 501-512 (2002)). Although the interaction between CRY2 and CIB1 occurs on a subsecond timescale (Kennedy, M. J. et al. Rapid blue-light-mediated induction of protein interactions in living cells. Nature methods 7, 973-975, doi:10.1038/nmeth.1524 (2010)), LITE-mediated activation is likely to be limited by the inherent kinetics of transcription. Applicants investigated the on-kinetics of LITE-mediated Neurog2 expression by measuring mRNA levels during a time course of light stimulation from 30 min to 24 h (FIG. 36C). Relative levels of Neurog2 mRNA increased considerably as early as 30 min after the onset of light stimulation and rose steadily until saturating at 12 h with a roughly 20-fold upregulation compared to GFP-transfected negative controls. Similarly, Applicants assessed the off-kinetics of the system by stimulating cells for 6 h and measuring the level of Neurog2 transcripts at multiple time points after ceasing illumination (FIG. 36D). Neurog2 mRNA levels briefly increased up to 30 min post-stimulation, an effect that may have resulted from residual CRY2PHR-CIB1 dimerization or from previously recruited RNA polymerases. Thereafter, Neurog2 expression declined with a half-life of ˜3 h, demonstrating that transcripts return to natural levels in the absence of light stimulation. In contrast, a small-molecule inducible TALE system based on the plant hormone abcisic acid receptor (Liang, F.-S., Ho, W. Q. & Crabtree, G. R. Engineering the ABA Plant Stress Pathway for Regulation of Induced Proximity. Sci. Signal. 4, rs2-, doi:10.1126/scisignal.2001449 (2011)) exhibited slower on- and off-kinetics (FIG. 44), potentially limited by drug diffusion, metabolism, or clearance.

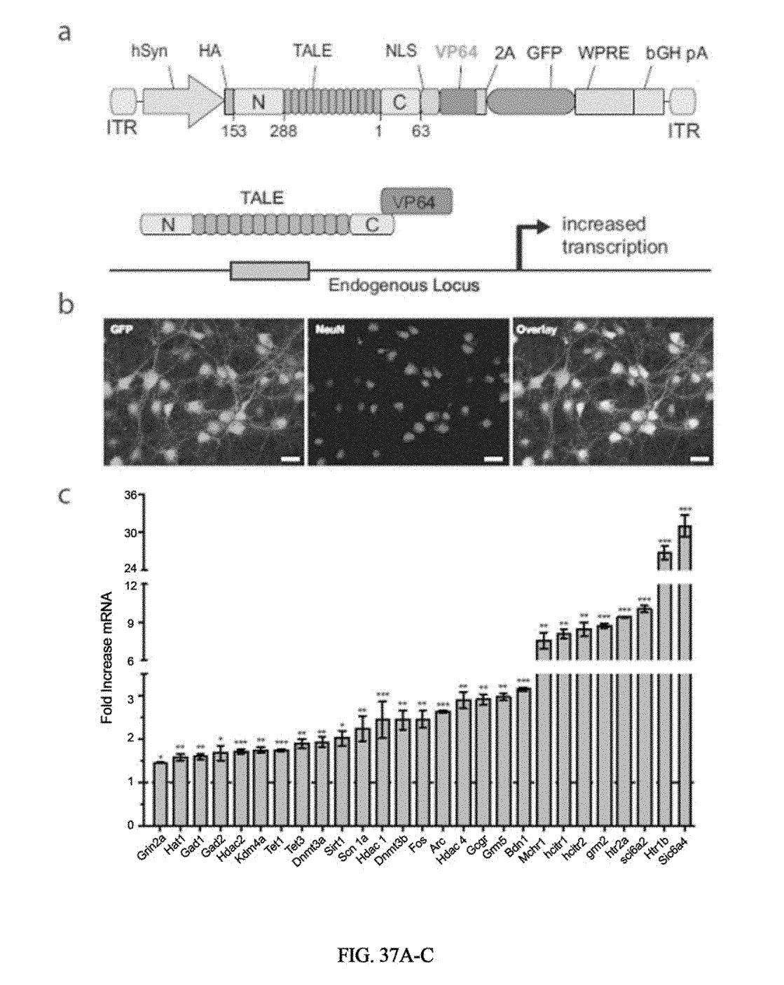

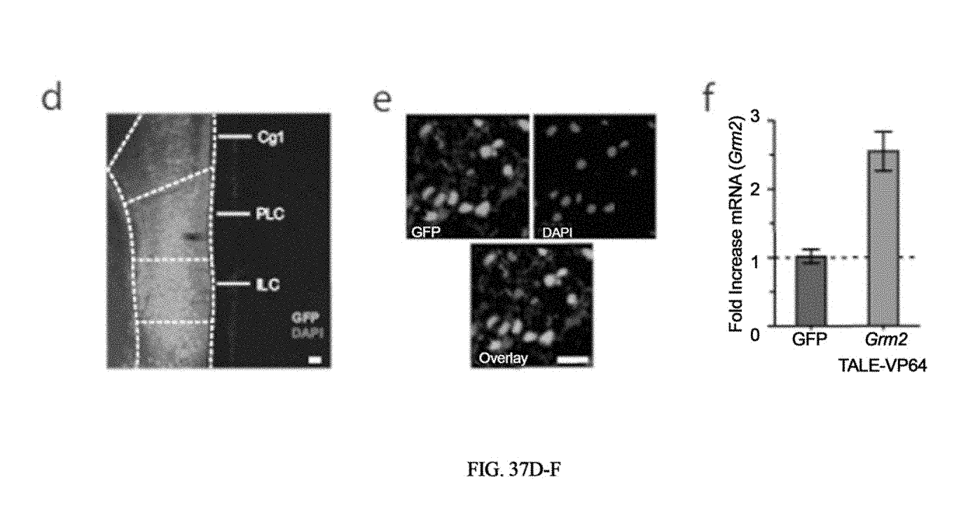

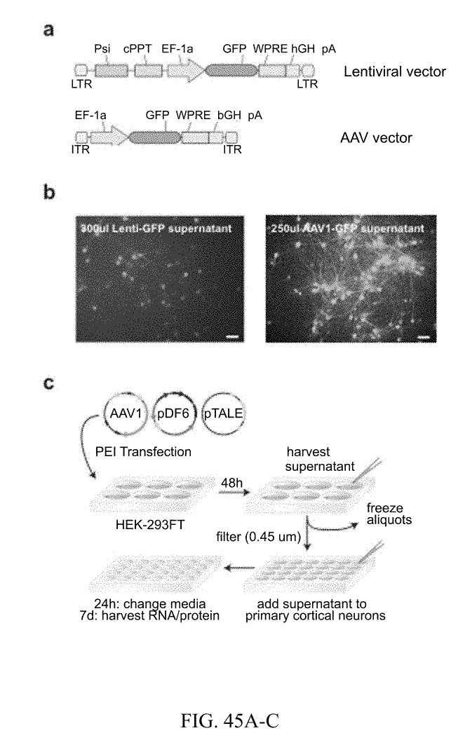

Applicants next explored the utility of LITEs for neuronal applications via viral transduction. Applicants developed an adeno-associated virus (AAV)-based vector for the delivery of TALE genes and a simplified process for AAV production (FIGS. 37A and B, FIG. 45, and Example 11). The ssDNA-based genome of AAV is less susceptible to recombination, providing an advantage over lentiviral vectors (Holkers, M. et al. Differential integrity of TALE nuclease genes following adenoviral and lentiviral vector gene transfer into human cells. Nucleic acids research 41, e63, doi:10.1093/nar/gks1446 (2013)).

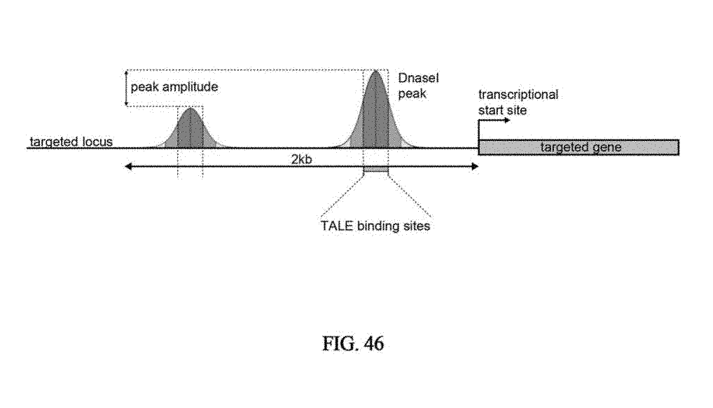

To characterize AAV-mediated TALE delivery for modulating transcription in primary mouse cortical neurons, Applicants constructed a panel of TALE-VP64 transcriptional activators targeting 28 murine loci in all, including genes involved in neurotransmission or neuronal differentiation, ion channel subunits, and genes implicated in neurological diseases. DNase I-sensitive regions in the promoter of each target gene provided a guide for TALE binding sequence selections (FIG. 46). Applicants confirmed that TALE activity can be screened efficiently using Applicants' AAV-TALE production process (FIG. 45) and found that TALEs chosen in this fashion and delivered into primary neurons using AAV vectors activated a diverse array of gene targets to varying extents (FIG. 37C). Moreover, stereotactic delivery of AAV-TALEs mediated robust expression in vivo in the mouse prefrontal cortex (FIG. 37D, E). Expression of TALE(Grm2)-VP64 in the mouse infralimbic cortex (ILC) induced a 2.5-fold increase in Grm2 mRNA levels compared to GFP-injected controls (FIG. 37F).

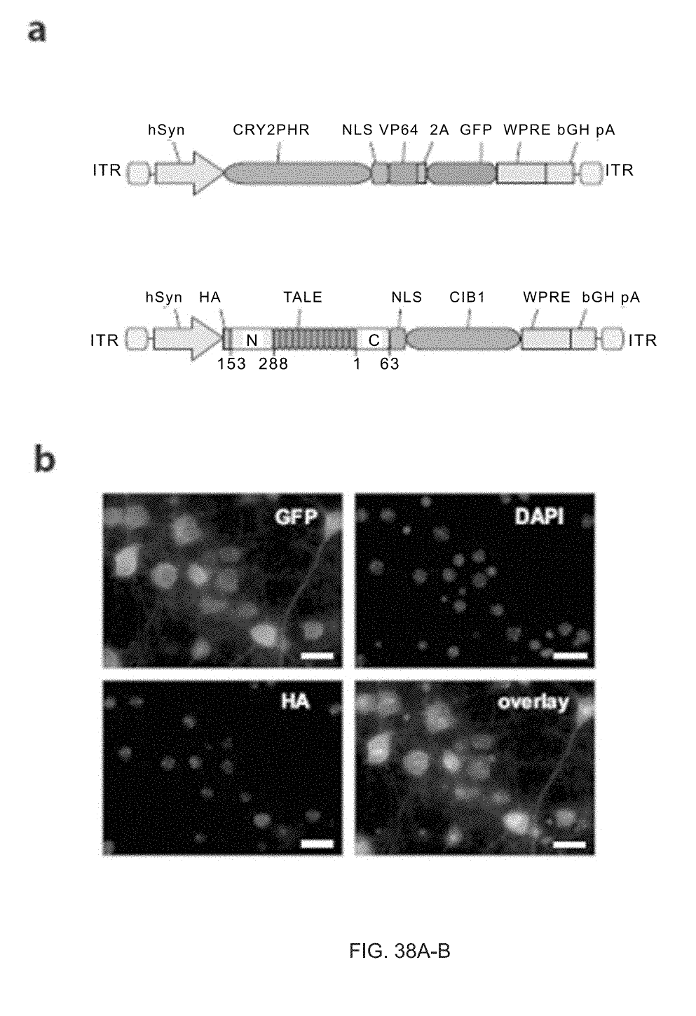

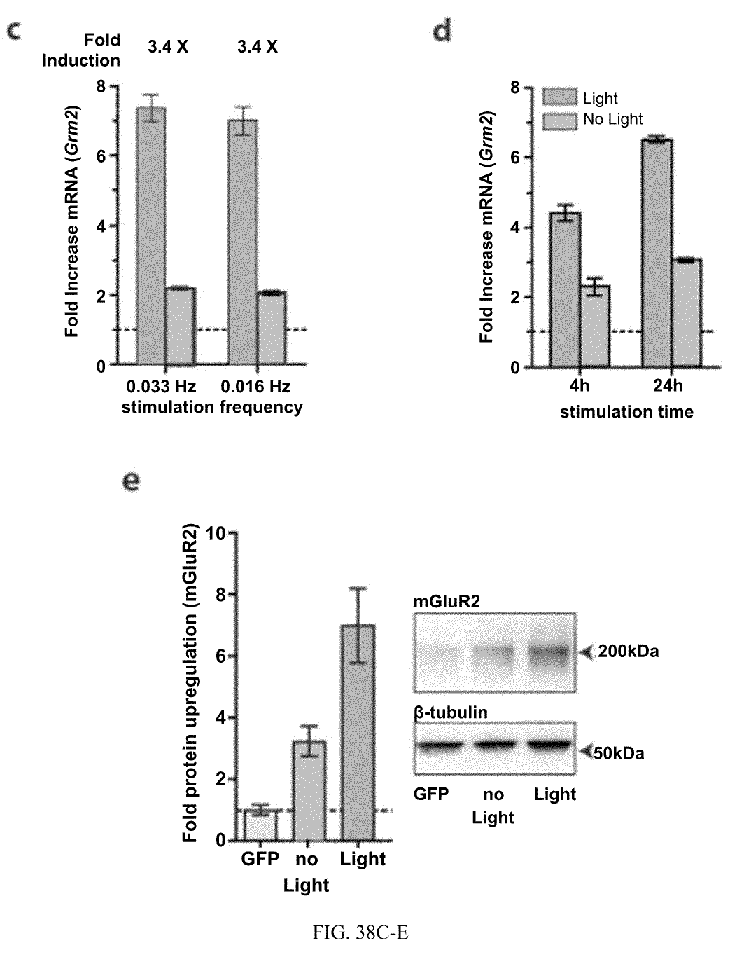

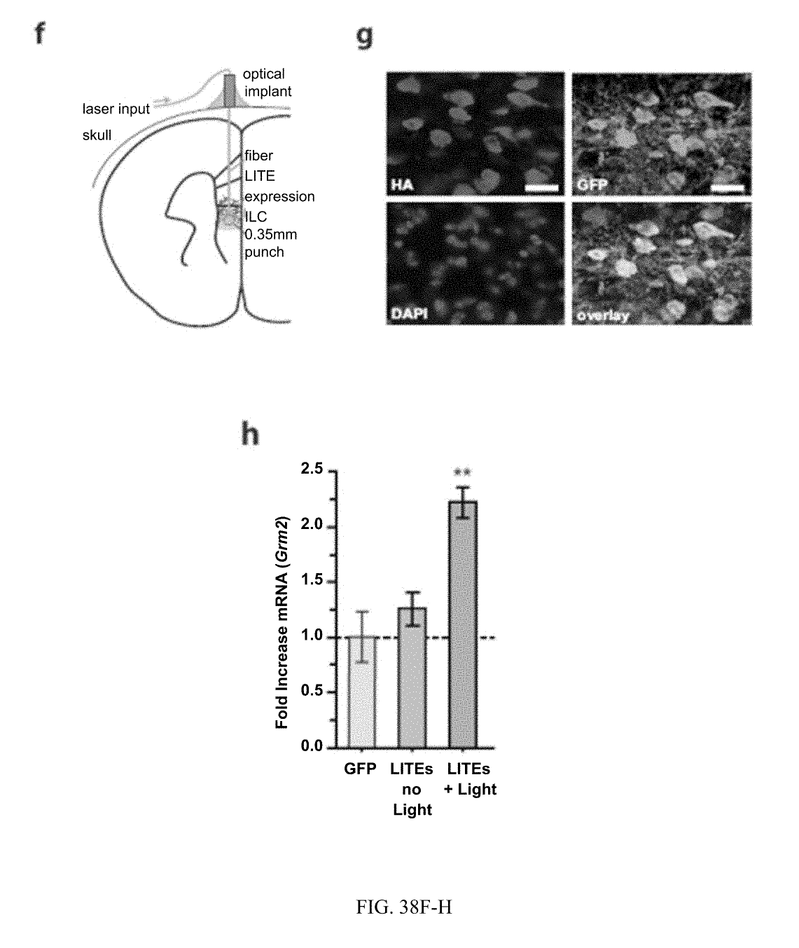

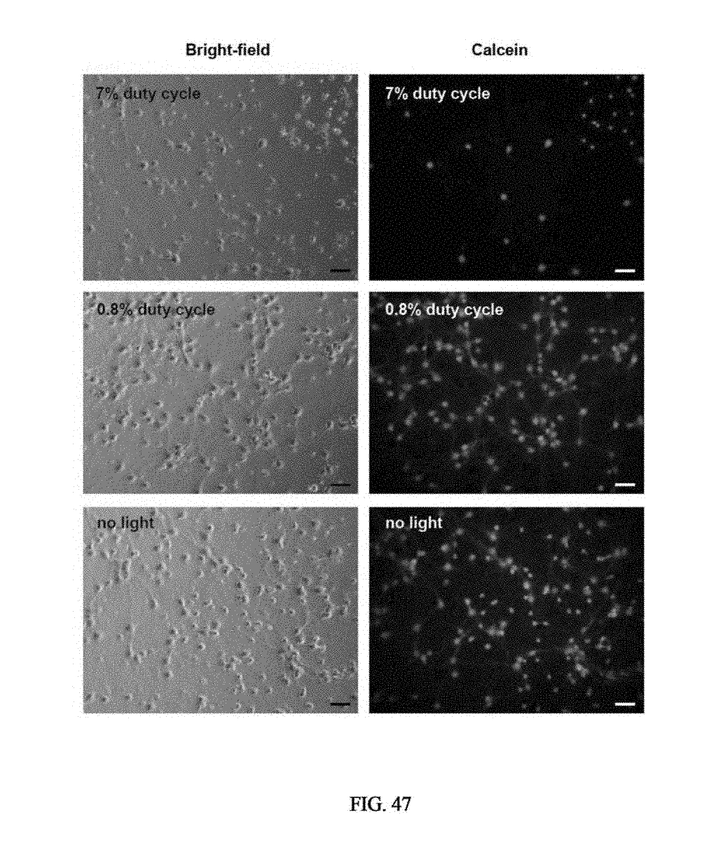

Having delivered TALE activators into cultured primary neurons, Applicants next sought to use AAV as a vector for the delivery of LITE components. To do so, Applicants needed to ensure that the total viral genome size of each recombinant AAV, with the LITE transgenes included, did not exceed the packaging limit of 4.8 kb (Wu, Z., Yang, H. & Colosi, P. Effect of Genome Size on AAV Vector Packaging. Mol Ther 18, 80-86 (2009)). Applicants shortened the TALE N- and C-termini (keeping 136 aa in the N-terminus and 63 aa in the C-terminus) and exchanged the CRY2PHR (1.5kb) and CIB1 (1kb) domains (TALE-CIB1 and CRY2PHR-VP64; FIG. 38A). These LITEs were delivered into primary cortical neurons via co-transduction by a combination of two AAV vectors (FIG. 38B; delivery efficiencies of 83-92% for individual components with >80% co-transduction efficiency). Applicants tested a Grm2-targeted LITE at 2 light pulsing frequencies with a reduced duty cycle of 0.8% to ensure neuron health (FIG. 47). Both stimulation conditions achieved a ˜7-fold light-dependent increase in Grm2 mRNA levels (FIG. 38C). Further study verified that substantial target gene expression increases could be attained quickly (4-fold upregulation within 4 h; FIG. 38D). In addition, Applicants observed significant upregulation of mGluR2 protein after stimulation, demonstrating that changes effected by LITEs at the mRNA level translate to the protein level (p<0.01 vs GFP control, p<0.05 vs no-light condition; FIG. 38E).

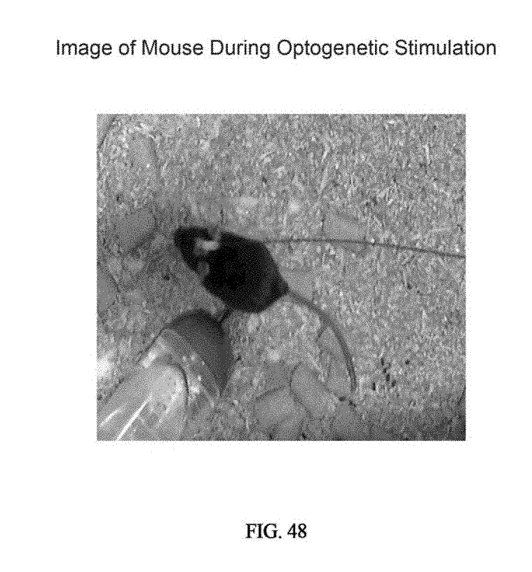

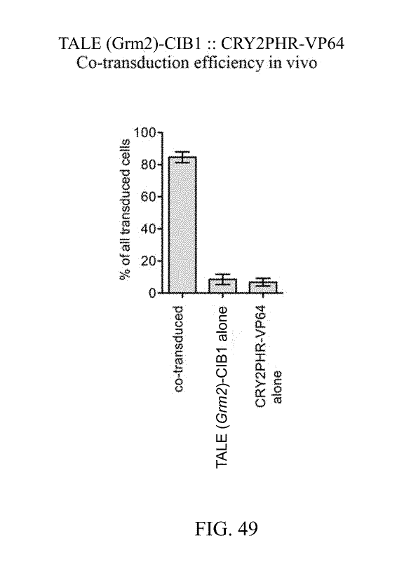

To apply the LITE system in vivo, Applicants stereotactically delivered a 1:1 mixture of high concentration AAV vectors (10¹² DNAseI resistant particles/mL) carrying the Grm2-targeting TALE-CIB1 and CRY2PHR-VP64 LITE components into ILC of wildtype C57BL/6 mice. To provide optical stimulation of LITE-expressing neurons in vivo, Applicants implanted a fiber optic cannula at the injection site (FIG. 38F and FIG. 48) (Zhang, F. et al. Optogenetic interrogation of neural circuits: technology for probing mammalian brain structures. Nat Protoc 5, 439-456, doi:10.1038/nprot.2009.226 (2010)). Neurons at the injection site were efficiently co-transduced by both viruses, with >80% of transduced cells expressing both TALE(Grm2)-CIB1 and CRY2PHR-VP64 (FIG. 38G and FIG. 49). 8 days post-surgery, Applicants stimulated the ILC of behaving mice by connecting a solid-state 473 nm laser to the implanted fiber cannula. Following a 12 h stimulation period (5 mW, 0.8% duty cycle using 0.5 s light pulses at 0.0167 Hz), brain tissue from the fiber optic cannula implantation site was analyzed (FIG. 38H) for changes in Grm2 mRNA. Applicants observed a significant increase in Grm2 mRNA after light stimulation compared with unstimulated ILC (p≤0.01). Taken together, these results confirm that LITEs enable optical control of endogenous gene expression in cultured neurons and in vivo.

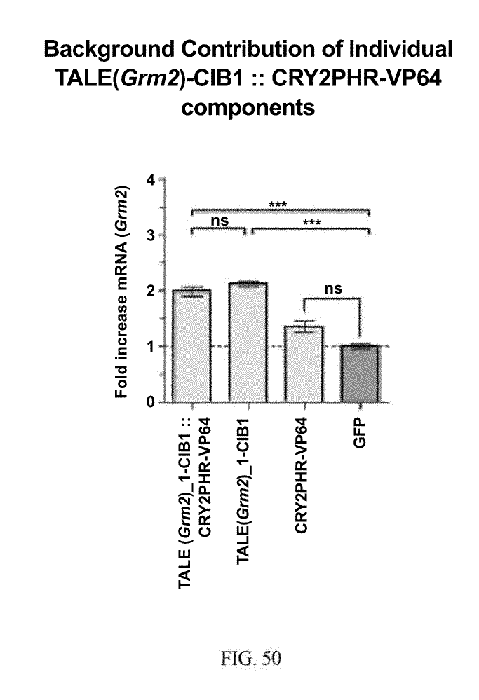

Due to the persistence of basal up-regulation observed in the no-light condition of in vivo LITE activators, Applicants undertook another round of optimization, aiming to identify and attenuate the source of the background and improve the efficiency of light-mediated gene induction (light/no-light ratio of gene expression). Neurons expressing only the LITE targeting component TALE-CIB1 produced Grm2 mRNA increases similar to those found in unstimulated neurons expressing both LITE components (both p≤0.001 versus GFP controls), while the effector component CRY2PHR-VP64 alone did not significantly affect transcription (p>0.05, FIG. 50), implying that the background transcriptional activation caused by LITE could arise solely from the DNA targeting component.

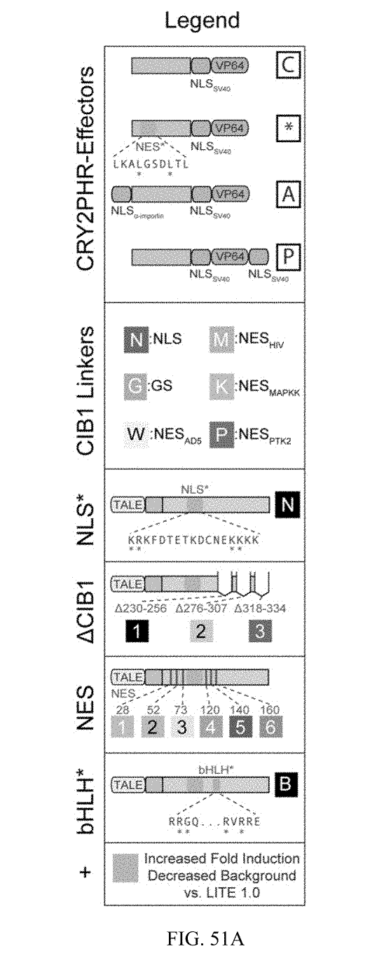

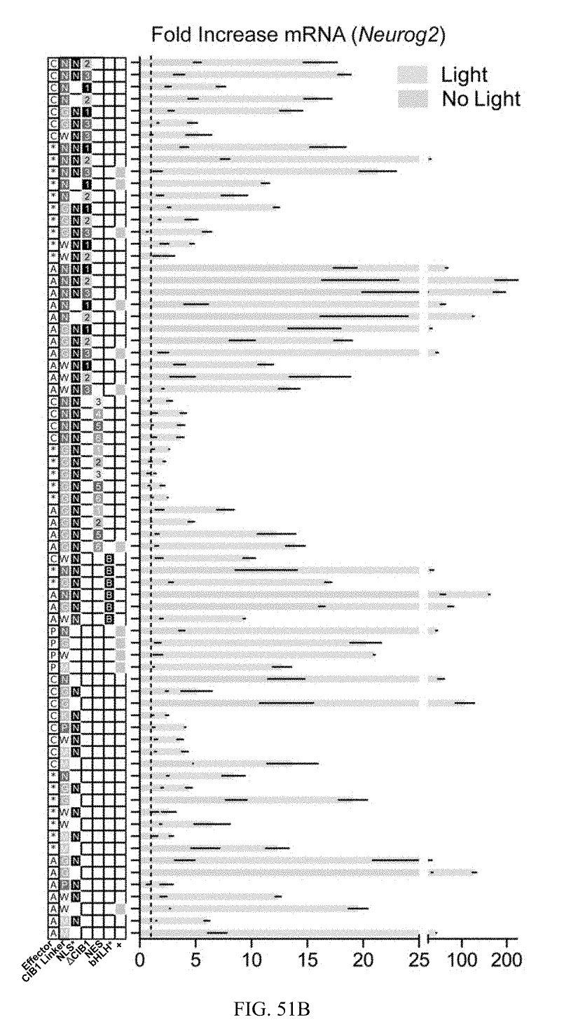

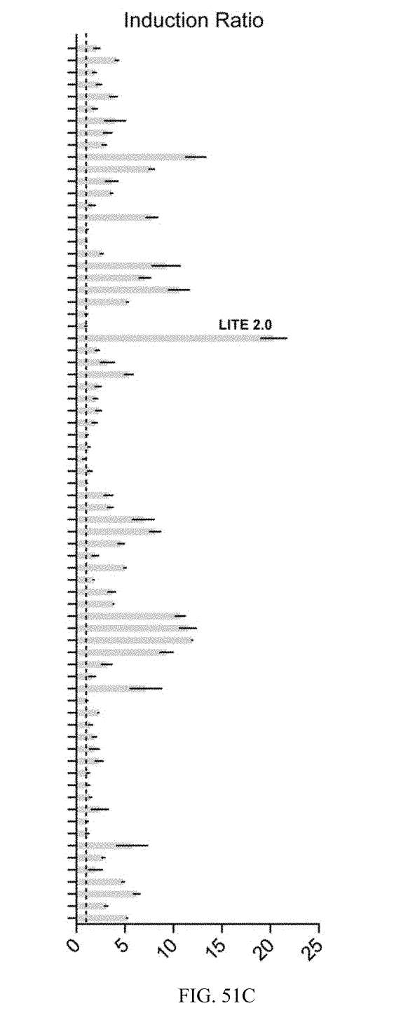

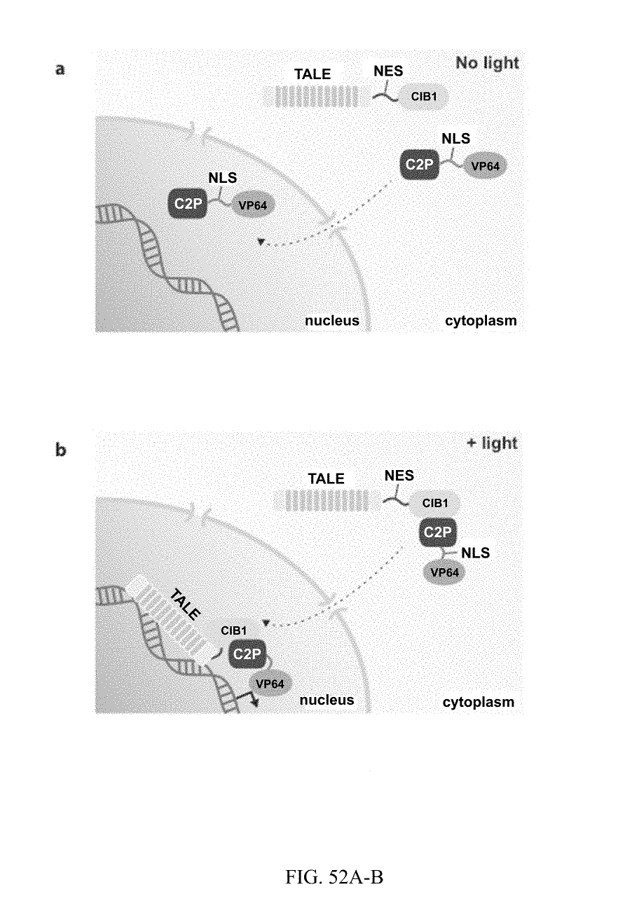

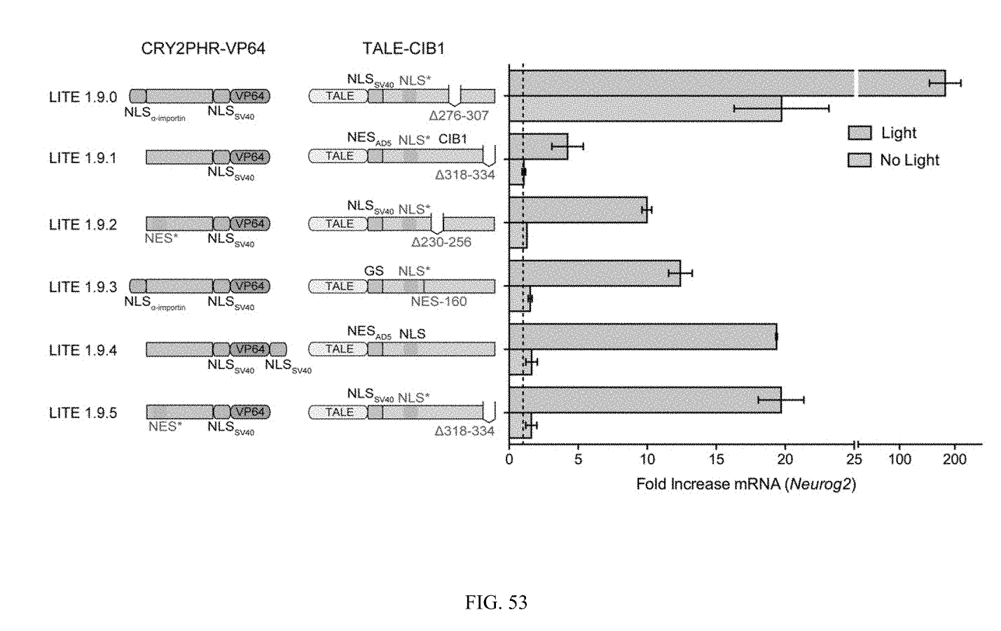

Accordingly, Applicants carried out a comprehensive screen to reduce the basal target up-regulation caused by TALE-CIB1 (FIG. 51). The optimization focused on two strategies: First, CIB1 is a plant transcription factor and may have intrinsic regulatory effects even in mammalian cells (Liu, H. et al. Photoexcited CRY2 Interacts with CIB1 to Regulate Transcription and Floral Initiation in Arabidopsis. Science 322, 1535-1539, doi:10.1126/science.1163927 (2008)). Applicants sought to eliminate these effects by deleting three CIB1 regions conserved amongst the basic helix-loop-helix transcription factors of higher plants (FIG. 51). Second, Applicants aimed to prevent TALE-CIB1 from binding the target locus in the absence of light. To achieve this, Applicants engineered TALE-CIB1 to localize in cytoplasm until light-induced dimerization with the NLS-containing CRY2PHR-VP64 (FIG. 52). To test both strategies independently or in combination, Applicants evaluated 73 distinct LITE architectures and identified 12 effector-targeting domain pairs (denoted by the “+” column in FIG. 51 and FIG. 53) with both improved light-induction efficiency and reduced overall baseline (fold mRNA increase in the no-light condition compared with the original LITE1.0; p<0.05). One architecture incorporating both strategies, designated LITE2.0, demonstrated the highest light induction (light/no-light=20.4) and resulted in greater than 6-fold reduction of background activation compared with the original architecture (FIG. 38I). Another—LITE1.9.1—produced a minimal background mRNA increase (1.06) while maintaining four-fold light induction (FIG. 53).

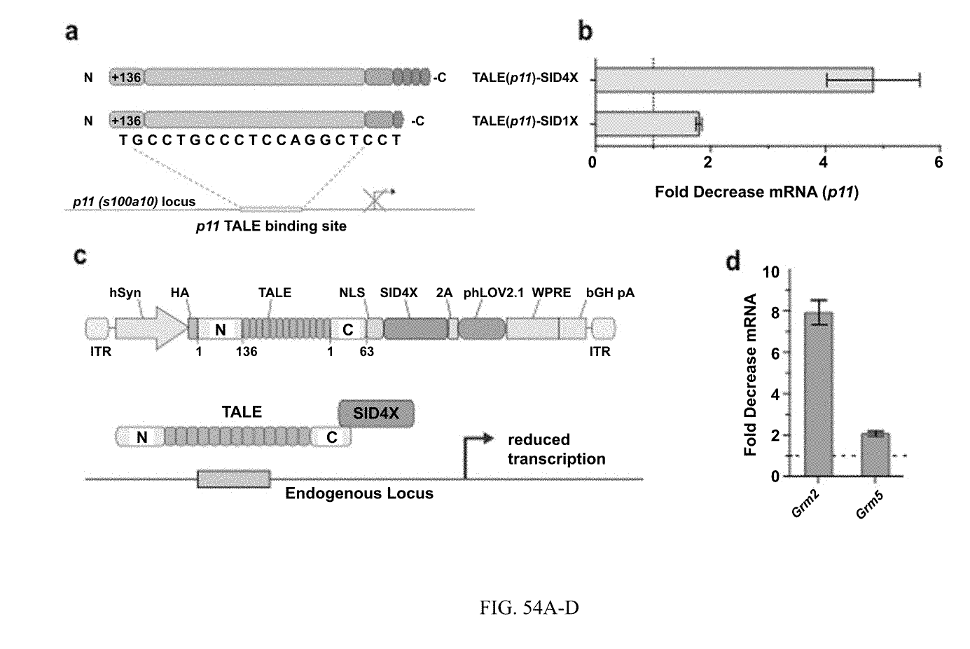

Applicants sought to further expand the range of processes accessible by TALE and LITE modulation. Endogenous transcriptional repression is often mediated by chromatin modifying enzymes such as histone methyltransferases (HMTs) and deacetylases (HDACs). Applicants have previously shown that the mSin3 interaction domain (SID), part of the mSin3-HDAC complex, can be fused with TALE in order to down regulate target genes in 293FT cells (Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proceedings of the National Academy of Sciences of the United States of America 95, 14628-14633 (1998) and Cong, L., Zhou, R., Kuo, Y.-c., Cunniff, M. & Zhang, F. Comprehensive interrogation of natural TALE DNA-binding modules and transcriptional repressor domains. Nat Commun 3, 968, (2012)). Hoping to further improve this TALE repressor, Applicants reasoned that four repeats of SID—analogous to the quadruple VP16 tandem repeat architecture of VP64 (Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proceedings of the National Academy of Sciences of the United States of America 95, 14628-14633 (1998))—might augment its potency to repress gene transcription. Indeed, TALE-SID4X constructs were twice as effective as TALE-SID in 293FT cells (FIGS. 54A and 54B) and also mediated efficient gene repression in neurons (FIGS. 54C and 54D).

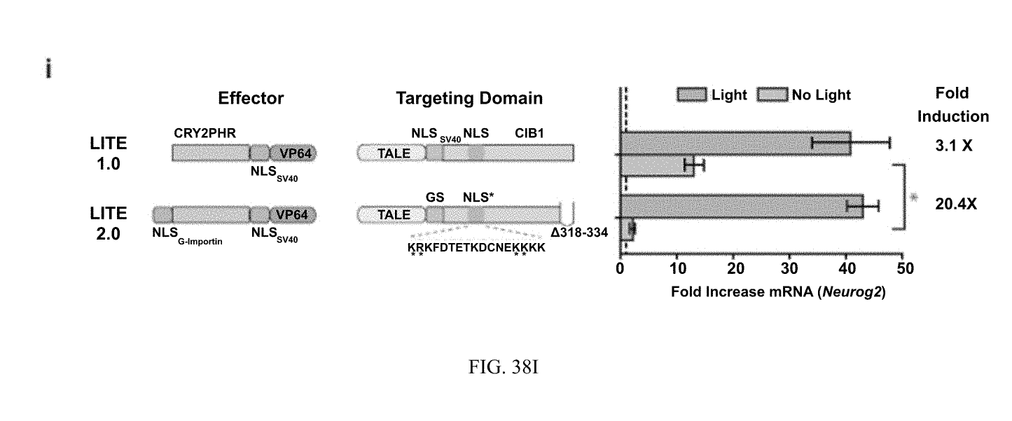

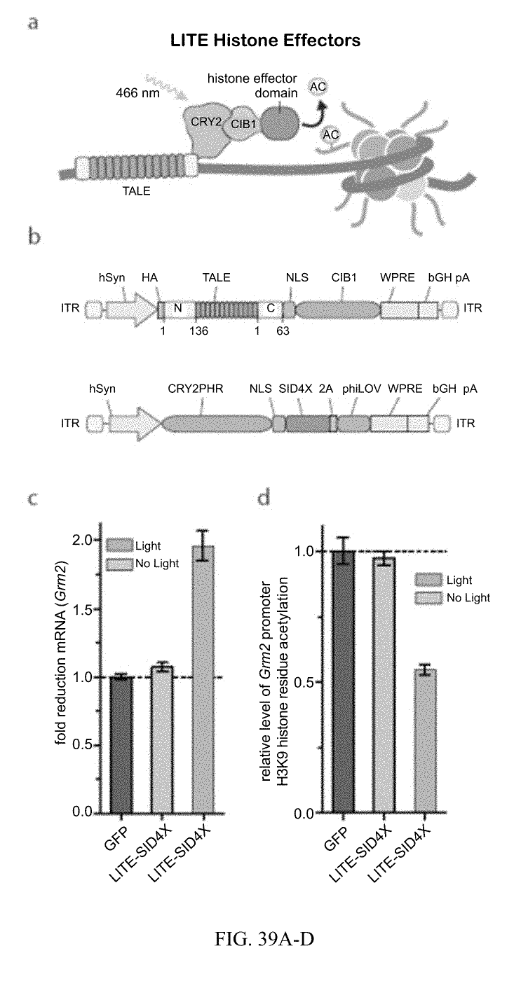

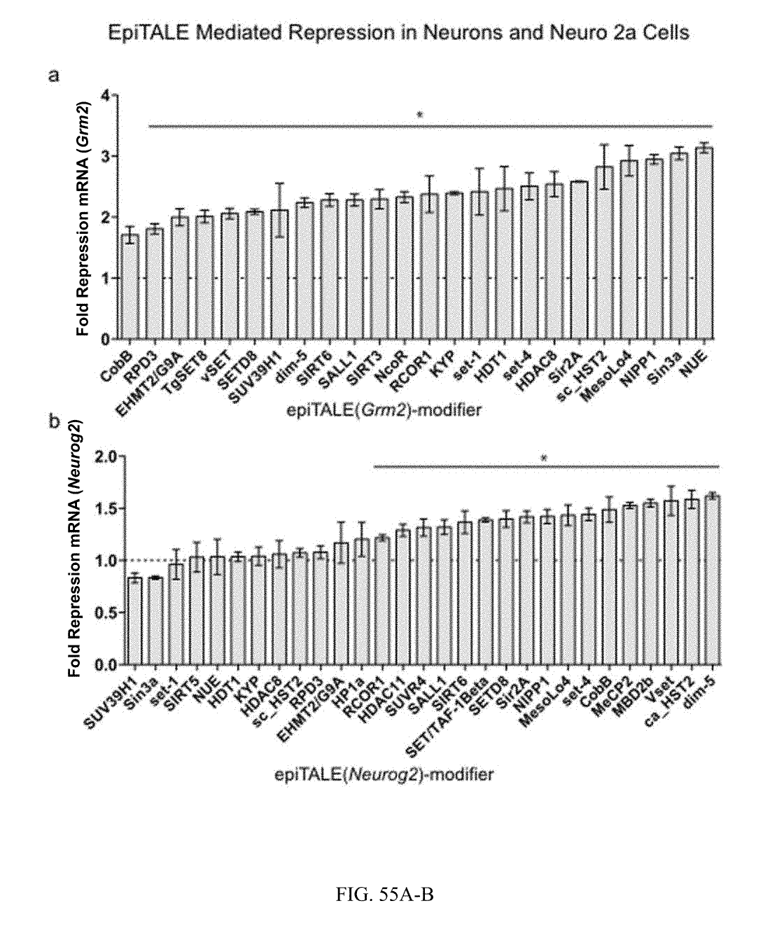

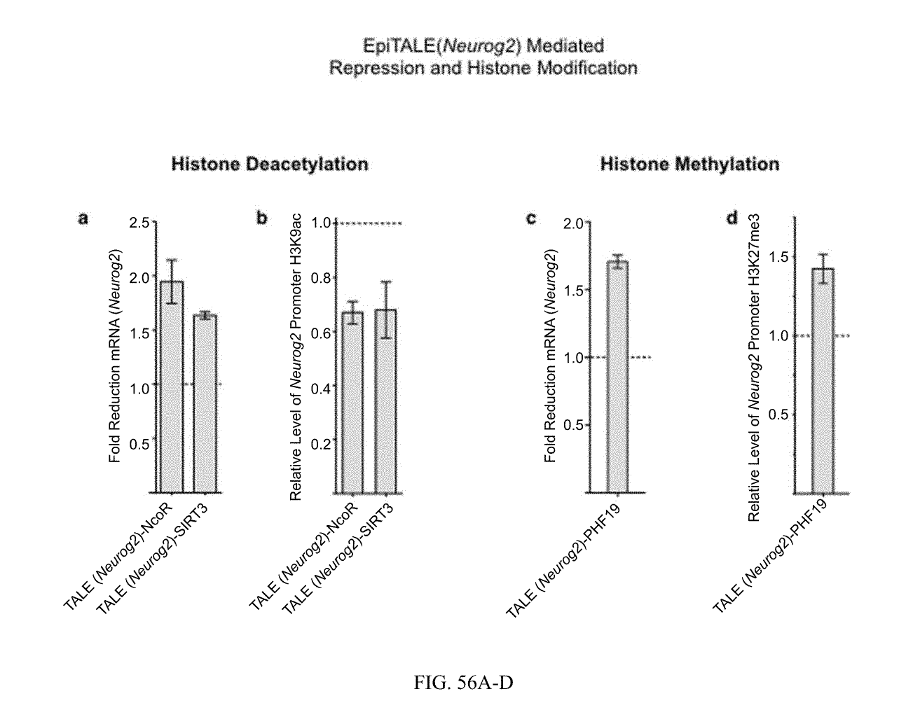

Applicants hypothesized that TALE-mediated targeting of histone effectors to endogenous loci could induce specific epigenetic modifications, enabling the interrogation of epigenetic as well as transcriptional dynamics (FIG. 39A). Applicants generated CRY2PHR-SID4X constructs and demonstrated light-mediated transcription repression of Grm2 in neurons (FIG. 39B and FIG. 39C), concomitant with ˜2-fold reduction in H3K9 acetylation at the targeted Grm2 promoter (FIG. 39D). In an effort to expand the diversity of histone residue targets for locus specific histone modification, Applicants derived a set of repressive histone effector domains from the literature (Table 6). Drawn from across a wide phylogenetic spectrum, the domains included HDACs, histone methyltransferases (HMTs), and histone acetyltransferase (HAT) inhibitors, as well as HDAC and HMT recruiting proteins. Preference was given to proteins and functional truncations of small size to facilitate efficient AAV packaging. The resulting epigenetic-modifying TALE-histone effector fusion constructs (epiTALEs) were tested in primary neurons and Neuro 2a cells for their ability to repress Grm2 and Neurog2 transcription, respectively (FIG. 39E, FIG. 39F and FIG. 55). In primary neurons, 23 out of 24 epiTALEs successfully repressed transcription of grm2 using the statistical criteria of p<0.05. Similarly, epiTALE expression in Neuro 2a cells led to decreased Neurog2 expression for 20 of the 32 histone effector domains tested (p<0.05). A subset of promising epiTALEs were expressed in primary neurons and Neuro 2a cells and relative histone residue mark levels in the targeted endogenous promoter were quantified by ChIP-RT-qPCR (FIG. 39G, FIG. 39H and FIG. 56). In primary neurons or Neuro 2a cells, levels of H3K9me1, H4K20me3, H3K27me3, H3K9ac, and H4K8ac were altered by epiTALEs derived from, respectively, KYP (A. thaliana), TgSET8 (T. gondii), NUE and PHF19 (C. trachomatis and H. sapiens), Sin3a, Sirt3 and NcoR, (all H. sapiens) and hdac8, RPD3, and Sir2a (X. laevis, S. cerevisiae, P. falciparum). These domains provide a ready source of epigenetic effectors to expand the range of transcriptional and epigenetic controls by LITE.

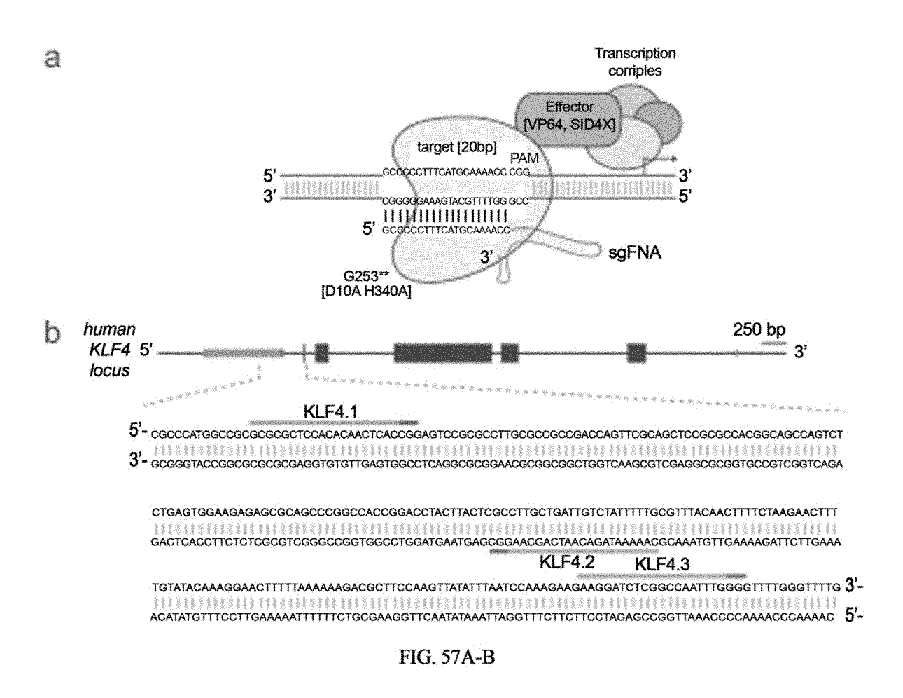

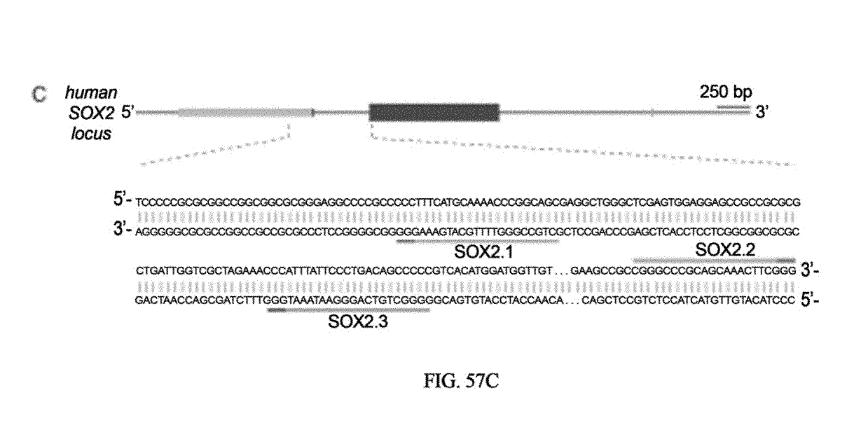

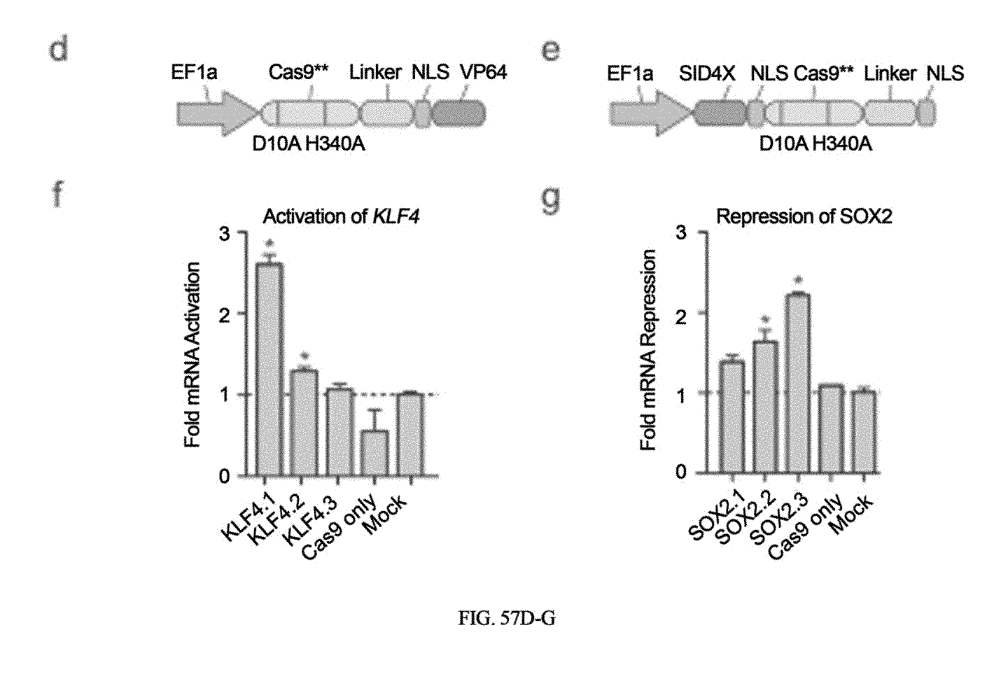

The ability to achieve spatiotemporally precise in vivo gene regulation in heterogeneous tissues such as the brain would allow researchers to ask questions about the role of dynamic gene regulation in processes as diverse as development, learning, memory, and disease progression. LITEs can be used to enable temporally precise, spatially targeted, and bimodal control of endogenous gene expression in cell lines, primary neurons, and in the mouse brain in vivo. The TALE DNA binding component of LITEs can be customized to target a wide range of genomic loci, and other DNA binding domains such as the RNA-guided Cas9 enzyme (Cong, L. et al. Multiplex genome engineering using CRISPR/Cas systems. Science 339, 819-823 (2013)) may be used in lieu of TALE to enable versatile locus-specific targeting (FIG. 57). Novel modes of LITE modulation can also be achieved by replacing the effector module with new functionalities such as epigenetic modifying enzymes (de Groote, M. L., Verschure, P. J. & Rots, M. G. Epigenetic Editing: targeted rewriting of epigenetic marks to modulate expression of selected target genes. Nucleic acids research 40, 10596-10613, doi:10.1093/nar/gks863 (2012)). Therefore the LITE system enables a new set of capabilities for the existing optogenetic toolbox and establishes a highly generalizable and versatile platform for altering endogenous gene regulation using light.

Methods Summary.

LITE constructs were transfected into in Neuro 2A cells using GenJet. AAV vectors carrying TALE or LITE constructs were used to transduce mouse primary embryonic cortical neurons as well as the mouse brain in vivo. RNA was extracted and reverse transcribed and mRNA levels were measured using TaqMan-based RT-qPCR. Light emitting diodes or solid-state lasers were used for light delivery in tissue culture and in vivo respectively.

Design and Construction of LITEs.

All LITE constructs sequences can be found in Example 11.

Neuro 2a Culture and Experiments.

Neuro 2a cells (Sigma-Aldrich) were grown in media containing a 1:1 ratio of OptiMEM (Life Technologies) to high-glucose DMEM with GlutaMax and Sodium Pyruvate (Life Technologies) supplemented with 5% HyClone heat-inactivated FBS (Thermo Scientific), 1% penicillin/streptomycin (Life Technologies), and passaged at 1:5 every 2 days. 120,000 cells were plated in each well of a 24-well plate 18-20 h prior to transfection. 1 h before transfection, media was changed to DMEM supplemented with 5% HyClone heat-inactivated FBS and 1% penicillin/streptomycin. Cells were transfected with 1.0 μg total of construct DNA (at equimolar ratios) per well with 1.5 μL of GenJet (SignaGen Laboratories) transfection reagent according to the manufacturer's instructions. Media was exchanged 24 h and 44 h post-transfection and light stimulation was started at 48 h. Stimulation parameters were: 5 mW/cm2, 466 nm, 7% duty cycle (1 s light pulse 0.067 Hz) for 24 h unless indicated otherwise in figure legends. RNA was extracted using the RNeasy kit (Qiagen) according to manufacturer's instructions and 1 μg of RNA per sample was reverse-transcribed using qScript (Quanta Biosystems). Relative mRNA levels were measured by quantitative real-time PCR (qRT-PCR) using TaqMan probes specific for the targeted gene as well as GAPDH as an endogenous control (Life Technologies, see Table 3 for Taqman probe IDs). ΔΔCt analysis was used to obtain fold-changes relative to negative controls transduced with GFP only and subjected to light stimulation. Toxicity experiments were conducted using the LIVE/DEAD assay kit (Life Technologies) according to instructions.

AAV Vector Production.

293FT cells (Life Technologies) were grown in antibiotic-free D10 media (DMEM high glucose with GlutaMax and Sodium Pyruvate, 10% heat-inactivated Hyclone FBS, and 1% 1M HEPES) and passaged daily at 1:2-2.5. The total number of passages was kept below 10 and cells were never grown beyond 85% confluence. The day before transfection, 1×10⁶ cells in 21.5 mL of D10 media were plated onto 15 cm dishes and incubated for 18-22 hours or until ˜80% confluence. For use as a transfection reagent, 1 mg/mL of PEI “Max” (Polysciences) was dissolved in water and the pH of the solution was adjusted to 7.1. For AAV production, 10.4 μg of pDF6 helper plasmid, 8.7 μg of pAAV1 serotype packaging vector, and 5.2 μg of pAAV vector carrying the gene of interest were added to 434 μL of serum-free DMEM and 130 μL of PEI “Max” solution was added to the DMEM-diluted DNA mixture. The DNA/DMEM/PEI cocktail was vortexed and incubated at room temperature for 15 min. After incubation, the transfection mixture was added to 22 mL of complete media, vortexed briefly, and used to replace the media for a 15 cm dish of 293FT cells. For supernatant production, transfection supernatant was harvested at 48 h, filtered through a 0.45 μm PVDF filter (Millipore), distributed into aliquots, and frozen for storage at −80° C.

Primary Cortical Neuron Culture.

Dissociated cortical neurons were prepared from C57BL/6N mouse embryos on E16 (Charles River Labs). Cortical tissue was dissected in ice-cold HBSS—(50 mL 10×HBSS, 435 mL dH₂O, 0.3 M HEPES pH 7.3, and 1% penicillin/streptomycin). Cortical tissue was washed 3× with 20 mL of ice-cold HBSS and then digested at 37° C. for 20 min in 8 mL of HBSS with 240 μL of 2.5% trypsin (Life Technologies). Cortices were then washed 3 times with 20 mL of warm HBSS containing 1 mL FBS. Cortices were gently triturated in 2 ml of HBSS and plated at 150,000 cells/well in poly-D-lysine coated 24-well plates (BD Biosciences). Neurons were maintained in Neurobasal media (Life Technologies), supplemented with 1×B27 (Life Technologies), GlutaMax (Life Technologies) and 1% penicillin/streptomycin.

Primary Neuron Transduction and Light Stimulation Experiments.

Primary cortical neurons were transduced with 250 μL of AAV1 supernatant on DIV 5. The media and supernatant were replaced with regular complete neurobasal the following day. Neurobasal was exchanged with Minimal Essential Medium (Life Technologies) containing 1×B27, GlutaMax (Life Technologies) and 1% penicillin/streptomycin 6 days after AAV transduction to prevent formation of phototoxic products from HEPES and riboflavin contained in Neurobasal during light stimulation.

Light stimulation was started 6 days after AAV transduction (DIV 11) with an intensity of 5 mW/cm², duty cycle of 0.8% (250 ms pulses at 0.033 Hz or 500 ms pulses at 0.016 Hz), 466 nm blue light for 24 h unless indicated otherwise in figure legends. RNA extraction and reverse transcription were performed using the Cells-to-Ct kit according to the manufacturers instructions (Life Technologies). Relative mRNA levels were measured by quantitative real-time PCR (qRT-PCR) using TaqMan probes as described above for Neuro 2a cells.

Immunohistochemistry of Primary Neurons.

For immunohistochemistry of primary neurons, cells were plated on poly-D-lysine/laminin coated coverslips (BD Biosciences) after harvesting. AAV1-transductions were performed as described above. Neurons were fixed 7 days post-transduction with 4% paraformaldehyde (Sigma Aldrich) for 15 min at RT. Blocking and permeabilization were performed with 10% normal goat serum (Life Technologies) and 0.5% Triton-X₁₀₀ (Sigma-Aldrich) in DPBS (Life Technologies) for 1 h at room temperature. Neurons were incubated with primary antibodies overnight at 4° C., washed 3× with DPBS and incubated with secondary antibodies for 90 min at RT. For antibody providers and concentrations used, see Table 4. Coverslips were finally mounted using Prolong Gold Antifade Reagent with DAPI (Life Technologies) and imaged on an Axio Scope A. 1 (Zeiss) with an X-Cite 120Q light source (Lumen Dynamics). Image were acquired using an AxioCam MRm camera and AxioVision 4.8.2.

Western Blots.

For preparation of total protein lysates, primary cortical neurons were harvested after light stimulation (see above) in ice-cold lysis buffer (RIPA, Cell Signaling; 0.1% SDS, Sigma-Aldrich; and cOmplete ultra protease inhibitor mix, Roche Applied Science). Cell lysates were sonicated for 5 min at ‘M’ setting in a Bioruptor sonicator (Diagenode) and centrifuged at 21,000×g for 10 min at 4° C. Protein concentration was determined using the RC DC protein assay (Bio-Rad). 30-40 μg of total protein per lane was separated under non-reducing conditions on 4-15% Tris-HCl gels (Bio-Rad) along with Precision Plus Protein Dual Color Standard (Bio-Rad) After wet electrotransfer to polyvinylidene difluoride membranes (Millipore) and membrane blocking for 45 min in 5% BLOT-QuickBlocker (Millipore) in Tris-buffered saline (TBS, Bio-Rad), western blots were probed with anti-mGluR2 (Abcam, 1:1.000) and anti-α-tubulin (Sigma-Aldrich 1:20,000) overnight at 4° C., followed by washing and anti-mouse-IgG HRP antibody incubation (Sigma-Aldrich, 1:5,000-1:10,000). For further antibody details see Table 4. Detection was performed via ECL Western blot substrate (SuperSignal West Femto Kit, Thermo Scientific). Blots were imaged with an AlphaImager (Innotech) system, and quantified using ImageJ software 1.46r.

Production of Concentrated and Purified AAV1/2 Vectors.

Production of concentrated and purified AAV for stereotactic injection in-vivo was done using the same initial steps outlined above for production of AAV1 supernatant. However, for transfection, equal ratios of AAV1 and AAV2 serotype plasmids were used instead of AAV1 alone. 5 plates were transfected per construct and cells were harvested with a cell-scraper 48 h post transfection. Purification of AAV1/2 particles was performed using HiTrap heparin affinity columns (GE Healthcare) (McClure, C., Cole, K. L., Wulff, P., Klugmann, M. & Murray, A. J. Production and titering of recombinant adeno-associated viral vectors. J Vis Exp, e3348, doi:10.3791/3348 (2011)). Applicants added a second concentration step down to a final volume of 100 μl per construct using an Amicon 500 μl concentration column (100 kDa cutoff, Millipore) to achieve higher viral titers. Titration of AAV was performed by qRT-PCR using a custom Taqman probe for WPRE (Life Technologies). Prior to qRT-PCR, concentrated AAV was treated with DNaseI (New England Biolabs) to achieve a measurement of DNaseI-resistant particles only. Following DNaseI heat-inactivation, the viral envelope was degraded by proteinase K digestion (New England Biolabs). Viral titer was calculated based on a standard curve with known WPRE copy numbers.

Stereotactic Injection of AAV1/2 and Optical Implant.

All animal procedures were approved by the MIT Committee on Animal Care. Adult (10-14 weeks old) male C57BL/6N mice were anaesthetized by intraperitoneal (i.p.) injection of Ketamine/Xylazine (100 mg/kg Ketamine and 10 mg/kg Xylazine) and pre-emptive analgesia was given (Buprenex, 1 mg/kg, i.p.). Craniotomy was performed according to approved procedures and 1 μl of AAV1/2 was injected into ILC at 0.35/1.94/−2.94 (lateral, anterior and inferior coordinates in mm relative to bregma). During the same surgical procedure, an optical cannula with fiber (Doric Lenses) was implanted into ILC unilaterally with the end of the optical fiber located at 0.35/1.94/−2.64 relative to bregma. The cannula was affixed to the skull using Metabond dental cement (Parkell Inc) and Jet denture repair (Lang dental) to build a stable cone around it. The incision was sutured and proper post-operative analgesics were administered for three days following surgery.

Immunohistochemistry on ILC Brain Sections.

Mice were injected with a lethal dose of Ketamine/Xylazine anaesthetic and transcardially perfused with PBS and 4% paraformaldehyde (PFA). Brains were additionally fixed in 4% PFA at 4° C. overnight and then transferred to 30% sucrose for cryoprotection overnight at room temperature. Brains were then transferred into Tissue-Tek Optimal Cutting Temperature (OCT) Compound (Sakura Finetek) and frozen at −80° C. 18 μm sections were cut on a cryostat (Leica Biosystems) and mounted on Superfrost Plus glass slides (Thermo Fischer). Sections were post-fixed with 4% PFA for 15 min, and immunohistochemistry was performed as described for primary neurons above.

Light Stimulation and mRNA Level Analysis in ILC.

8 days post-surgery, awake and freely moving mice were stimulated using a 473 nm laser source (OEM Laser Systems) connected to the optical implant via fiber patch cables and a rotary joint. Stimulation parameters were the same as used on primary neurons: 5 mW (total output), 0.8% duty cycle (500 ms light pulses at 0.016 Hz) for a total of 12 h. Experimental conditions, including transduced constructs and light stimulation are listed in Table 5.

After the end of light stimulations, mice were euthanized using CO₂ and the prefrontal cortices (PFC) were quickly dissected on ice and incubated in RNA later (Qiagen) at 4° C. overnight. 200 μm sections were cut in RNA later at 4° C. on a vibratome (Leica Biosystems). Sections were then frozen on a glass coverslide on dry ice and virally transduced ILC was identified under a fluorescent stereomicroscope (Leica M165 FC). A 0.35 mm diameter punch of ILC, located directly ventrally to the termination of the optical fiber tract, was extracted (Harris uni-core, Ted Pella). The brain punch sample was then homogenized using an RNase-free pellet-pestle grinder (Kimble Chase) in 50 μl Cells-to-Ct RNA lysis buffer and RNA extraction, reverse transcription and qRT-PCR was performed as described for primary neuron samples.

Chromatin Immunoprecipitation.

Neurons or Neuro2a cells were cultured and transduced or transfected as described above. ChIP samples were prepared as previously described (Blecher-Gonen, R. et al. High-throughput chromatin immunoprecipitation for genome-wide mapping of in vivo protein-DNA interactions and epigenomic states. Nature protocols 8, 539-554 (2013)) with minor adjustments for the cell number and cell type. Cells were harvested in 24-well format, washed in 96-well format, and transferred to microcentrifuge tubes for lysis. Sample cells were directly lysed by water bath sonication with the Biorupter sonication device for 21 minutes using 30 s on/off cycles (Diagenode). qPCR was used to assess enrichment of histone marks at the targeted locus.

Statistical Analysis.

All experiments were performed with a minimum of two independent biological replicates. Statistical analysis was performed with Prism (GraphPad) using Student's two-tailed t-test when comparing two conditions, ANOVA with Tukey's post-hoc analysis when comparing multiple samples with each other, and ANOVA with Dunnett's post-hoc analysis when comparing multiple samples to the negative control.

### Example 11

### Supplementary Information to Example 10: Optical Control of Endogenous Mammalian Transcription

Photostimulation Hardware—In Vitro.

In vitro light stimulation experiments were performed using a custom built LED photostimulation device. All electronic elements were mounted on a custom printed circuit board (ExpressPCB). Blue LEDs with peaks 466 nm (model #: YSL-R542B5C-All, China Young Sun LED Technology; distributed by SparkFun Electronics as ‘LED—Super Bright Blue’ COM-00529), were arrayed in groups of three aligned with the wells of a Corning 24-well plate. LED current flow was regulated by a 25 mA DynaOhm driver (LEDdymanics #4006-025). Columns of the LED array were addressed by TTL control (Fairchild Semiconductor PN2222BU-ND) via an Arduino UNO microcontroller board. Light output was modulated via pulse width modulation. Light output was measured from a distance of 80 mm above the array utilizing a Thorlabs PM100D power meter and S120VC photodiode detector. In order to provide space for ventilation and to maximize light field uniformity, an 80 mm tall ventilation spacer was placed between the LED array and the 24-well sample plate. Fans (Evercool EC5015M12CA) were mounted along one wall of the spacer unit, while the opposite wall was fabricated with gaps to allow for increased airflow.

Quantification of LIVE/DEAD® Assay Using ImageJ Software.

Images of LIVE/DEAD (Life Technologies) stained cells were captured by fluorescence microscopy and processed as follows: Background was subtracted (Process→Subtract Background). A threshold based on fluorescence area was set to ensure accurate identification of cell state (Image→Adjust→Threshold). A segmentation analysis was performed to enable automated counting of individual cells (Process→Binary→Watershed). Finally, debris signals were filtered and cells were counted (Analyze→Analyze Particles). Toxicity was determined as the percentage of dead cells.

Chemically-Inducible TALEs.

Neuro2A cells were grown in a medium containing a 1:1 ratio of OptiMEM (Life Technologies) to high-glucose DMEM with GlutaMax and Sodium Pyruvate (Life Technologies) supplemented with 5% HyClone heat-inactivated FBS (Thermo Scientific), 1% penicillin/streptomycin (Life Technologies) and 25 mM HEPES (Sigma Aldrich). 150,000 cells were plated in each well of a 24-well plate 18-24 hours prior to transfection. Cells were transfected with 1 μg total of construct DNA (at equimolar ratios) per well and 2 μL of Lipofectamine 2000 (Life Technologies) according to the manufacturer's recommended protocols. Media was exchanged 12 hours post-transfection. For the kinetics test, chemical induction was started 24 hours post-transfection, when abscisic acid (ABA, Sigma Aldrich) was added to fresh media to a final concentration of 250 μM. RNA was extracted using the RNeasy kit (Qiagen) according to manufacturer's instructions and 1 μg of RNA per sample was reverse-transcribed using qScript (Quanta Biosystems). Relative mRNA levels were measured by quantitative real-time PCR (qRT-PCR) using Taqman probes specific for the targeted gene as well as mouse GAPDH as an endogenous control (Life Technologies, see Supplementary Table 2 for Taqman probe IDs). ΔΔCt analysis was used to obtain fold-changes relative to negative controls where cells were subjected to mock transfection with GFP.

Cas9 Transcriptional Effectors.

HEK 293FT cells were co-transfected with mutant Cas9 fusion protein and a synthetic guide RNA (sgRNA) using Lipofectamine 2000 (Life Technologies) 24 hours after seeding into a 24 well dish. 72 hours post-transfection, total RNA was purified (RNeasy Plus, Qiagen). 1 ug of RNA was reverse transcribed into cDNA (qScript, Quanta BioSciences). Quantitative real-time PCR was done according to the manufacturer's protocol (Life Technologies) and performed in triplicate using TaqMan Assays for hKlf4 (Hs00358836_m1), hSox2 (Hs01053049_s1), and the endogenous control GAPDH (Hs02758991_g1).

The hSpCas9 activator plasmid was cloned into a lentiviral vector under the expression of the hEF1a promoter (pLenti-EF1a-Cas9-NLS-VP64). The hSpCas9 repressor plasmid was cloned into the same vector (pLenti-EF1a-SID4x-NLS-Cas9-NLS). Guide sequences (20 bp) targeted to the KLF4 locus are: GCGCGCTCCACACAACTCAC (SEQ ID NO: 92), GCAAAAATAGACAATCAGCA (SEQ ID NO: 93), GAAGGATCTCGGCCAATTTG (SEQ ID NO: 94). Spacer sequences for guide RNAs targeted to the SOX2 locus are: GCTGCCGGGTTTTGCATGAA (SEQ ID NO: 95), CCGGGCCCGCAGCAAACTTC (SEQ ID NO: 96), GGGGCTGTCAGGGAATAAAT (SEQ ID NO: 97).

Optogenetic Actuators:

Microbial and plant-derived light-sensitive proteins have been engineered as optogenetic actuators, allowing optical control of cellular functions including membrane potential (Deisseroth, K. Optogenetics. Nature methods 8, 26-29, doi:10.1038/nmeth.f.324 (2011); Zhang, F. et al. The microbial opsin family of optogenetic tools. Cell 147, 1446-1457, doi:10.1016/j.cell.2011.12.004 (2011) and Yizhar, O., Fenno, L. E., Davidson, T. J., Mogri, M. & Deisseroth, K. Optogenetics in neural systems. Neuron 71, 9-34, doi:10.1016/j.neuron.2011.06.004 (2011)), intracellular biochemical signaling (Airan, R. D., Thompson, K. R., Fenno, L. E., Bernstein, H. & Deisseroth, K. Temporally precise in vivo control of intracellular signalling. Nature 458, 1025-1029, doi:10.1038/nature07926 (2009)), protein interactions (Levskaya, A., Weiner, O. D., Lim, W. A. & Voigt, C. A. Spatiotemporal control of cell signalling using a light-switchable protein interaction. Nature 461, 997-1001, doi:10.1038/nature08446 (2009); Yazawa, M., Sadaghiani, A. M., Hsueh, B. & Dolmetsch, R. E. Induction of protein-protein interactions in live cells using light. Nat Biotechnol 27, 941-945, doi:10.1038/nbt.1569 (2009); Strickland, D. et al. TULIPs: tunable, light-controlled interacting protein tags for cell biology. Nature methods 9, 379-384, doi:10.1038/nmeth.1904 (2012) and Kennedy, M. J. et al. Rapid blue-light-mediated induction of protein interactions in living cells. Nature methods 7, 973-975, doi:10.1038/nmeth.1524 (2010)), and heterologous gene expression (Yazawa, M., Sadaghiani, A. M., Hsueh, B. & Dolmetsch, R. E. Induction of protein-protein interactions in live cells using light. Nat Biotechnol 27, 941-945, doi:10.1038/nbt.1569 (2009); Kennedy, M. J. et al. Rapid blue-light-mediated induction of protein interactions in living cells. Nature methods 7, 973-975, doi:10.1038/nmeth.1524 (2010); Shimizu-Sato, S., Huq, E., Tepperman, J. M. & Quail, P. H. A light-switchable gene promoter system. Nat Biotechnol 20, 1041-1044, doi:10.1038/nbt734 (2002); Ye, H., Daoud-El Baba, M., Peng, R. W. & Fussenegger, M. A synthetic optogenetic transcription device enhances blood-glucose homeostasis in mice. Science 332, 1565-1568, doi:10.1126/science.1203535 (2011); Wang, X., Chen, X. & Yang, Y. Spatiotemporal control of gene expression by a light-switchable transgene system. Nature methods 9, 266-269, doi:10.1038/nmeth.1892 (2012) and Polstein, L. R. & Gersbach, C. A. Light-inducible spatiotemporal control of gene activation by customizable zinc finger transcription factors. J Am Chem Soc 134, 16480-16483, doi:10.1021/ja3065667 (2012)).

Ambient Light Exposure:

All cells were cultured at low light levels (<0.01 mW/cm²) at all times except during stimulation. These precautions were taken as ambient light in the room (0.1-0.2 mW/cm²) was found to significantly activate the LITE system (FIG. 36D). No special precautions were taken to shield animals from light during in vivo experiments—even assuming ideal propagation within the implanted optical fiber, an estimation of light transmission at the fiber terminal due to ambient light was <0.01 mW (based on 200 μm fiber core diameter and 0.22 numerical aperture).

Optimization of Light Stimulation Parameters in Neuro2A Cells:

To minimize near-UV induced cytotoxicity, Applicants selected 466 nm blue LEDs to activate TALE-CRY2, a wavelength slightly red-shifted from the CRY2 absorption maxima of 450 nm but still maintaining over 80% activity (Banerjee, R. et al. The signaling state of Arabidopsis cryptochrome 2 contains flavin semiquinone. J Biol Chem 282, 14916-14922, doi:10.1074/jbc.M700616200 (2007)) (FIG. 42). To minimize light exposure, Applicants selected a mild stimulation protocol (1 s light pulses at 0.067 Hz, ˜7% duty cycle). This was based on Applicants' finding that light duty cycle had no significant effect on LITE-mediated transcriptional activation over a wide range of duty cycle parameters (1.7% to 100% duty cycles, FIG. 41). Illumination with a range of light intensities from 0 to 10 mW/cm² revealed that Ngn2 mRNA levels increased as a function of intensity up to 5 mW/cm². However, increases in Ngn2 mRNA levels declined at 10 mW/cm² (FIG. 36C), suggesting that higher intensity light may have detrimental effects on either LITE function or on cell physiology. To better characterize this observation, Applicants performed an ethidium homodimer-1 cytotoxicity assay with a calcein counterstain for living cells and found a significantly higher percentage of ethidium-positive cells at the higher stimulation intensity of 10 mW/cm². Conversely, the ethidium-positive cell count from 5 mW/cm² stimulation was indistinguishable from unstimulated controls. Thus 5 mW/cm² appeared to be optimal for achieving robust LITE activation while maintaining low cytotoxicity.

Reduction of Light-Induced Toxicity in Primary Neurons:

Initial application of LITEs in neurons revealed that cultured neurons were much more sensitive to blue light than Neuro 2a cells. Stimulation parameters that Applicants previously optimized for Neuro 2a cells (466 nm, 5 mW/cm² intensity, 7% duty cycle with 1 s light pulse at 0.067 Hz for a total of 24 h) caused >50% toxicity in primary neurons. Applicants therefore tested survival with a lower duty cycle, as Applicants had previously observed that a wide range of duty cycles had little effect on LITE-mediated transcriptional activation (FIG. 41). A reduced duty cycle of 0.8% (0.5 s light pulses at 0.0167 Hz) at the same light intensity (5 mW/cm²) was sufficient to maintain a high survival rate that was indistinguishable from that of unstimulated cultures (FIG. 47).

Light Propagation and Toxicity in In Vivo Experiments:

Previous studies have investigated the propagation efficiency of different wavelengths of light in brain tissue. For 473 nm light (wavelength used in this study), there was a >90% attenuation after passing through 0.35 mm of tissue (Witten, Ilana B. et al. Recombinase-Driver Rat Lines: Tools, Techniques, and Optogenetic Application to Dopamine-Mediated Reinforcement. Neuron 72, 721-733, doi:http://dx.doi.org/1016/i.neuron.2011.10.028 (2011)). An estimated 5 mW/cm² light power density was estimated based on a tissue depth of 0.35 mm of tissue (the diameter of brain punch used in this study) and a total power output of 5 mW. The light stimulation duty cycle used in vivo was the same (0.8%, 0.5 s at 0.0167 Hz) as that used for primary neurons (FIG. 47).

CRY2 Absorption Spectrum:

An illustration of the absorption spectrum of CRY2 was shown in FIG. 42. The spectrum showed a sharp drop in absorption above 480 nm (Banerjee, R. et al. The Signaling State of Arabidopsis Cryptochrome 2 Contains Flavin Semiquinone. Journal of Biological Chemistry 282, 14916-14922, doi:10.1074/jbc.M700616200 (2007)). Wavelengths >500 nm were virtually not absorbed, which could be useful for future multimodal optical control with yellow or red-light sensitive proteins.

Development of AAV1 Supernatant Process:

Traditional AAV particle generation required laborious production and purification processes, and made testing many constructs in parallel impractical (Grieger, J. C., Choi, V. W. & Samulski, R. J. Production and characterization of adeno-associated viral vectors. Nat Protoc 1, 1412-1428, doi:10.1038/nprot.2006.207 (2006)). In this study, a simple yet highly effective process of AAV production using filtered supernatant from transfected 293FT cells (FIG. 43). Recent reported indicate that AAV particles produced in 293FT cells could be found not only it the cytoplasm but also at considerable amounts in the culture media (Lock M, A. M., Vandenberghe L H, Samanta A, Toelen J, Debyser Z, Wilson J M. Rapid, Simple, and Versatile Manufacturing of Recombinant Adeno-Associated Viral Vectors at Scale. Human Gene Therapy 21, 1259-1271, doi:10.1089/hum.2010.055 (2010)). The ratio of viral particles between the supernatant and cytosol of host cells varied depending on the AAV serotype, and secretion was enhanced if polyethylenimine (PEI) was used to transfect the viral packaging plasmids (Lock M, A. M., Vandenberghe L H, Samanta A, Toelen J, Debyser Z, Wilson J M. Rapid, Simple, and Versatile Manufacturing of Recombinant Adeno-Associated Viral Vectors at Scale. Human Gene Therapy 21, 1259-1271, doi:10.1089/hum.2010.055 (2010)). In the current study, it was found that 2×10⁵ 293FT cells transfected with AAV vectors carrying TALEs (FIG. 38A) and packaged using AAV1 serotype were capable of producing 250 μl of AAV1 at a concentration of 5.6±0.24×10¹⁰ DNAseI resistant genome copies (gc) per mL. 250 μl of filtered supernatant was able to transduce 150,000 primary cortical neurons at efficiencies of 80-90% (FIG. 38B and FIG. 43). This process was also successfully adapted to a 96-well format, enabling the production of 125 ul AAV1 supernatant from up to 96 different constructs in parallel. 35ul of supernatant can then be used to transduce one well of primary neurons cultured in 96-well format, enabling the transduction in biological triplicate from a single well.

TABLE 3
Product information for all Taqman probes (Life Technologies)

| Target | Species | Probe # |
|---|---|---|
| Ngn2 | mouse | Mm00437603_g1 |
| Grm5 (mGluR5) | mouse | Mm00690332_m1 |
| Grm2 (mGluR2) | mouse | Mm01235831_m1 |
| Grin2a (NMDAR2A) | mouse | Mm00433802_m1 |
| GAPD (GAPDH) | mouse | 4352932E |
| KLF4 | human | Hs00358836_m1 |
| GAPD (GAPDH) | human | 4352934E |
| WPRE | custom | |
| 5-HT1A | mouse | Mm00434106_s1 |
| 5-HT1B | mouse | Mm00439377_s1 |
| 5-HTT | mouse | Mm00439391_m1 |
| Arc | mouse | Mm00479619_g1 |
| BDNF | mouse | Mm04230607_s1 |
| c-Fos | mouse | Mm00487425_m1 |
| CBP/P300 | mouse | Mm01342452_m1 |
| CREB | mouse | Mm00501607_m1 |
| CRHR1 | mouse | Mm00432670_m1 |
| DNMT1 | mouse | Mm01151063_m1 |
| DNMT3a | mouse | Mm00432881_m1 |
| DNMT3b | mouse | Mm01240113_m1 |
| egr-1 (zif-268) | mouse | Mm00656724_m1 |
| Gad65 | mouse | Mm00484623_m1 |
| Gad67 | mouse | Mm00725661_s1 |
| GR (GCR, NR3C1) | mouse | Mm00433832_m1 |
| HAT1 | mouse | Mm00509140_m1 |
| HCRTR1 | mouse | Mm01185776_m1 |
| HCRTR2 | mouse | Mm01179312_m1 |
| HDAC1 | mouse | Mm02391771_g1 |
| HDAC2 | mouse | Mm00515108_m1 |
| HDAC4 | mouse | Mm01299557_m1 |
| JMJD2A | mouse | Mm00805000_m1 |
| M1 (CHRM1) | mouse | Mm00432509_s1 |
| MCH-R1 | mouse | Mm00653044_m1 |
| NET (SLC6A2) | mouse | Mm00436661_m1 |
| NR2B subunit | mouse | Mm00433820_m1 |
| OXTR | mouse | Mm01182684_m1 |
| Scn1a | mouse | Mm00450580_m1 |
| SIRT1 | mouse | Mm00490758_m1 |
| Tet1 | mouse | Mm01169087_m1 |
| Tet2 | mouse | Mm00524395_m1 |
| Tet3 | mouse | Mm00805756_m1 |

TABLE 4
Clone, product numbers and concentrations for antibodies used in this study
Primary Antibodies

| Target | Host | Clone # | Manufacturer | Product # | IsoType | Concentration | |
|---|---|---|---|---|---|---|---|
| mGluR2 | mouse | mG2Na-s | Abcam | Ab15672 | IgG | 1:1000 | |
| α-tubulin | mouse | B-5-1-2 | Sigma-Aldrich | T5168 | IgG1 | 1:20000 | |
| NeuN | mouse | A60 | Millipore | MAB377 | IgG1 | 1:200 | |
| HA (Alexa | mouse | 6E2 | Cell Signaling | 3444 | IgG1 | 1:100 | |
| Fluor 594 | | | | | | | |
| GFP | chicken | polyclonal | Aves Labs | GFP-1020 | IgY | 1:500 | |
| Target | Host | Conjugate | Manufacturer | Product # | Concentration | | |
| mouse IgG | goat | HRP | Sigma-Aldrich | A9917 | 1:5000-10000 | | |
| mouse IgG | goat | Alexa Fluor | Life | A11005 | 1:1000 | | |
| | | 594 | Technologies | | | | |
| chicken IgG | Goat | Alexa Fluor | Life | A11039 | 1:1000 | | |
| | | 488 | Technologies | | | | |
| Target | Host | Epitope | Manufacturer | Product # | IsoType | Concentration | |
| H3K9me1 | mouse | 1-18 | Millipore | 17-680 | IgG | 2 | μl/IP |
| H3K9me2 | mouse | 1-18 | Millipore | 17-681 | IgG | 4 | μl/IP |
| H3K9Ac | rabbit | polyclonal | Millipore | 17-658 | IgG | 3 | μg/IP |
| H4K20me1 | rabbit | 15-24 | Millipore | 17-651 | IgG | 4 | μg/IP |
| H4K8Ac | rabbit | polyclonal | Millipore | 17-10099 | IgG | 1.5 | μl/IP |
| H4K20me3 | rabbit | 18-22 | Millipore | 17-671 | IgG | 7 | μl/IP |
| H3K27me3 | rabbit | polyclonal | Millipore | 17-622 | IgG | 4 | μg/IP |

TABLE 5
qPCR primers used for CHIP-qPCR

| target | Primers |
|---|---|
| Grm2 | Forward: CTGTGCTGAAGGATCTGGGG |
| promoter | (SEQ ID NO: 98) |
| | Reverse: ATGCTGCAGGCATAGGACAA |
| | (SEQ ID NO: 99) |
| Neurog2 | Forward: GAGGGGGAGAGGGACTAAAGA |
| promoter | (SEQ ID NO: 100) |
| | Reverse: GCTCTCCCTCCCCAGCTTA |
| | (SEQ ID NO: 101) |
| Myt-1 | Cell Signaling Technologies SimpleChIP ® |
| promoter | Mouse MYT-1 Promoter |
| control | Primers #8985 |
| RPL30 | Cell Signaling Technologies SimpleChIP ® |
| Intron 2 | Mouse RPL30 Intron 2 |
| control | Primers #7015 |

TABLE 6
genomic sequences targeted by TALEs

| | | |
|---|---|---|
| 5-HT1B | TATCTGAACTCTCC | SEQ ID NO: 102 |
| 5-HTT | TGTCTGTCTTGCAT | SEQ ID NO: 103 |
| Arc | TGGCTGTTGCCAGG | SEQ ID NO: 104 |
| BDNF | TACCTGGAGCTAGC | SEQ ID NO: 105 |
| c-Fos | TACACAGGATGTCC | SEQ ID NO: 106 |
| DNMT3a | TTGGCCCTGTGCAG | SEQ ID NO: 107 |
| DNMT3b | TAGCGCAGCGATCG | SEQ ID NO: 108 |
| gad65 | TATTGCCAAGAGAG | SEQ ID NO: 109 |
| gad67 | TGACTGGAACATAC | SEQ ID NO: 110 |
| GR (GCR, NR3C1) | TGATGGACTTGTAT | SEQ ID NO: 111 |
| HAT1 | TGGACCTTCTCCCT | SEQ ID NO: 112 |
| HCRTR1 | TAGGTCTCCTGGAG | SEQ ID NO: 113 |
| HCRTR2 | TGGCTCAGGAACTT | SEQ ID NO: 114 |
| HDAC1 | TTCTCTAAGCTGCC | SEQ ID NO: 115 |
| HDAC2 | TGAGCCCTGGAGGA | SEQ ID NO: 116 |
| HDAC4 | TGCCTAAGATGGAG | SEQ ID NO: 117 |
| JMJD2A | TGTAGTGAGTGTTC | SEQ ID NO: 118 |
| MCH-R1 | TGTCTAGGTGATGT | SEQ ID NO: 119 |
| NET | TCTCTGCTAGAAGG | SEQ ID NO: 120 |
| Scn1a | TCTAGGTCAAGTGT | SEQ ID NO: 121 |
| SIRT1 | TCCTCTGCTCCGCT | SEQ ID NO: 122 |
| tet1 | TCTAGGAGTGTAGC | SEQ ID NO: 123 |
| tet3 | TGCCTGGCTGCTGG | SEQ ID NO: 124 |
| 5-HT1B | TATCTGAACTCTCC | SEQ ID NO: 125 |
| Grm2 | TCAGAGCTGTCCTC | SEQ ID NO: 126 |
| Grm5 | TGCAAGAGTAGGAG | SEQ ID NO: 127 |
| 5-HT2A | TAGTGACTGATTCC | SEQ ID NO: 128 |
| Grin2a | TTGGAGGAGCACCA | SEQ ID NO: 129 |
| Neurog2 | TGAATGATGATAATAC | SEQ ID NO: 130 |

TABLE 7
Viral transduction and light stimulation parameters for in vivo LITE-
mediated activation of Grm2 in the mouse infralimbic cortex (ILC).
Grm2 mRNA levels in the ipsilateral LITE-expressing hemisphere are
compared with the contralateral mCherry-expressing control hemisphere
for all three experimental conditions shown in FIG. 39J.

| | ILC Hemisphere (ipsilateral) | | |
|---|---|---|---|
| | | Light | ILC Hemisphere |
| Experimental | | stimula- | (contralateral) |
| condition | AAV vector | tion | AAV vector |
| GFP | GFP | yes | mCherry |
| LITEs/no Light | TALE-CIB1::CRY2PHR- | no | mCherry |
| | VP64 | | |
| LITEs/+Light | TALE-CIB1::CRY2PHR- | yes | mCherry |
| | VP64 | | |

TABLE 8
HDAC Recruiter Effector Domains

| | | Substrate | | | Full | Selected | Final | |
|---|---|---|---|---|---|---|---|---|
| Subtype/ | | (if | Modification | | size | truncation | size | Catalytic |
| Complex | Name | known) | (if known) | Organism | (aa) | (aa) | (aa) | domain |
| Sin3a | MeCP2 | — | — | R. norvegicus | 492 | 207-492 | 286 | — |
| | | | | | | (Nan) | | |
| Sin3a | MBD2b | — | — | H. sapiens | 262 | 45-262 | 218 | — |
| | | | | | | (Boeke) | | |
| Sin3a | Sin3a | — | — | H. sapiens | 1273 | 524-851 | 328 | 627-829: |
| | | | | | | (Laherty) | | HDAC1 |
| | | | | | | | | interaction |
| NcoR | NcoR | — | — | H. sapiens | 2440 | 420-488 | 69 | — |
| | | | | | | (Zhang) | | |
| NuRD | SALL1 | — | — | M. musculus | 1322 | 1-93 | 93 | — |
| | | | | | | (Lauberth) | | |
| CoREST | RCOR1 | — | — | H. sapiens | 482 | 81-300 | 220 | — |
| | | | | | | (Gu, Ouyang) | | |

Nan, X. et al. Transcriptional repression by the methyl-CpG-binding protein MeCP2 involves a histone deacetylase complex. Nature 393, 386-389 (1998).
Boeke, J., Ammerpohl, O., Kegel, S., Moehren, U. & Renkawitz, R. The minimal repression domain of MBD2b overlaps with the methyl-CpG-binding domain and binds directly to Sin3A. Journal of Biological Chemistry 275, 34963-34967 (2000).
Laherty, C. D. et al. Histone deacetylases associated with the mSin3 corepressor mediate mad transcriptional repression. Cell 89, 349-356 (1997).
Zhang, J., Kalkum, M., Chait, B. T. & Roeder, R. G. The N-CoR-HDAC3 nuclear receptor corepressor complex inhibits the JNK pathway through the integral subunit GPS2. Molecular cell 9, 611-623 (2002).
Lauberth, S. M. & Rauchman, M. A conserved 12-amino acid motif in Sal11 recruits the nucleosome remodeling and deacetylase corepressor complex. Journal of Biological Chemistry 281, 23922-23931 (2006).
Gu, H. & Roizman, B. Herpes simplex virus-infected cell protein 0 blocks the silencing of viral DNA by dissociating histone deacetylases from the CoREST, ÄìREST complex.
Ouyang, J., Shi, Y., Valin, A., Xuan, Y. & Gill, G. Direct binding of CoREST1 to SUMO-2/3 contributes to gene-specific repression by the LSD1/CoREST1/HDAC complex. Molecular cell 34. 145-154 (2009)

TABLE 9
HDAC Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| HDAC I | HDAC8 | — | — | X. laevis | 325 | 1-325 | 325 | 1-272: |
| | | | | | | | | HDAC |
| HDAC I | RPD3 | — | — | S. cerevisiae | 433 | 19-340 | 322 | 19-331: |
| | | | | | | | (Vannier) | HDAC |
| HDAC IV | MesoLo4 | — | — | M. loti | 300 | 1-300 | 300 | — |
| | | | | | | (Gregoretti) | | |
| HDAC IV | HDAC11 | — | — | H. sapiens | 347 | 1-347 | 347 | 14-326: |
| | | | | | | (Gao) | | HDAC |
| HD2 | HDT1 | — | — | A. thaliana | 245 | 1-211 | 211 | — |
| | | | | | | (Wu) | | |
| SIRT I | SIRT3 | H3K9Ac | — | H. sapiens | 399 | 143-399 | 257 | 126-382: |
| | | H4K16Ac | | | | (Scher) | | SIRT |
| | | H3K56Ac | | | | | | |
| SIRT I | HST2 | — | — | C. albicans | 331 | 1-331 | 331 | — |
| | | | | | | (Hnisz) | | |
| SIRT I | CobB | — | — | E. coli (K12) | 242 | 1-242 | 242 | — |
| | | | | | | (Landry) | | |
| SIRT I | HST2 | — | — | S. cerevisiae | 357 | 8-298 | 291 | — |
| | | | | | | (Wilson) | | |
| SIRT III | SIRT5 | H4K8Ac | — | H. sapiens | 310 | 37-310 | 274 | 41-309: |
| | | H4K16Ac | | | | (Gertz) | | SIRT |
| SIRT III | Sir2A | — | — | P. falciparum | 273 | 1-273 | 273 | 19-273: |
| | | | | | | (Zhu) | | SIRT |
| SIRT IV | SIRT6 | H3K9Ac | — | H. sapiens | 355 | 1-289 | 289 | 35-274: |
| | | H3K56Ac | | | | (Tennen) | | SIRT |

Vannier, D., Balderes, D. & Shore, D. Evidence that the transcriptional regulators SIN3 and RPD3, and a novel gene (SDS3) with similar functions, are involved in transcriptional silencing in S. cerevisiae. Genetics 144, 1343-1353 (1996).
Gregoretti, I., Lee, Y.-M. & Goodson, H. V. Molecular evolution of the histone deacetylase family: functional implications of phylogenetic analysis. Journal of molecular biology 338, 17-31 (2004).
Gao, L., Cueto, M. A., Asselbergs, F. & Atadja, P. Cloning and functional characterization of HDAC11, a novel member of the human histone deacetylase family. Journal of Biological Chemistry 277, 25748-25755 (2002).
Wu, K., Tian, L., Malik, K., Brown, D. & Miki, B. Functional analysis of HD2 histone deacetylase homologues in Arabidopsis thaliana. The Plant Journal 22, 19-27 (2000).
Scher, M. B., Vaquero, A. & Reinberg, D. SirT3 is a nuclear NAD+-dependent histone deacetylase that translocates to the mitochondria upon cellular stress. Genes & development 21, 920-928 (2007).
Hnisz, D., Schwarzm√°ller, T. & Kuchler, K. Transcriptional loops meet chromatin: a dual, Äêlayer network controls white, Äìopaque switching in Candida albicans. Molecular microbiology 74, 1-15 (2009).
Landry, J. et al. The silencing protein SIR2 and its homologs are NAD-dependent protein deacetylases. Proceedings of the National Academy of Sciences 97, 5807-5811(2000).
Wilson, J. M., Le, V. Q., Zimmerman, C., Marmorstein, R. & Pillus, L. Nuclear export modulates the cytoplasmic Sir2 homologue Hst2. EMBO reports 7, 1247-1251 (2006).
Gertz, M. & Steegborn, C. Function and regulation of the mitochondrial Sirtuin isoform Sirt5 in Mammalia. Biochimica et Biophysica Acta (BBA)-Proteins and Proteomics 1804, 1658- 1665 (2010).
Zhu, A. Y. et al. Plasmodium falciparum Sir2A preferentially hydrolyzes medium and long chain fatty acyl lysine. ACS chemical biology 7, 155-159 (2011).
Tennen, R. I., Berber, E. & Chua, K. F. Functional dissection of SIRT6: identification of domains that regulate histone deacetylase activity and chromatin localization. Mechanisms of ageing and development 131, 185-192 (2010).

TABLE 10
Histone Methyltransferase (HMT) Effector Domains

| | | Substrate | | | Full | Selected | Final | |
|---|---|---|---|---|---|---|---|---|
| Subtype/ | | (if | Modification | | size | truncation | size | Catalytic |
| Complex | Name | known) | (if known) | Organism | (aa) | (aa) | (aa) | domain |
| SET | NUE | H2B, | — | C. trachomatis | 219 | 1-219 | 219 | — |
| | | H3, H4 | | | | (Pennini) | | |
| SET | vSET | — | H3K27me3 | P. bursaria | 119 | 1-119 | 119 | 4-112: |
| | | | | chlorella virus | | (Mujtaba) | | SET2 |
| SUV39 | EHMT2/G9A | H1.4K2, | H3K9me1/2, | M. musculus | 1263 | 969-1263 | 295 | 1025-1233: |
| family | | H3K9, | H1K25me1 | | | (Tachibana) | | preSET, SET, |
| | | H3K27 | | | | | | postSET |
| SUV39 | SUV39H1 | — | H3K9me2/3 | H. sapiens | 412 | 79-412 | 334 | 172-412: |
| | | | | | | (Snowden) | | preSET, SET, |
| | | | | | | | | postSET |
| Suvar3-9 | dim-5 | — | H3K9me3 | N. crassa | 331 | 1-331 | 331 | 77-331: |
| | | | | | | (Rathert) | | preSET, SET, |
| | | | | | | | | postSET |
| Suvar3-9 | KYP | — | H3K9me1/2 | A. thaliana | 624 | 335-601 | 267 | — |
| (SUVH | | | | | | | (Jackson) | |
| subfamily) | | | | | | | | |
| Suvar3-9 | SUVR4 | H3K9me1 | H3K9me2/3 | A. thaliana | 492 | 180-492 | 313 | 192-462: |
| (SUVR | | | | | | | (Thorst- | preSET, SET, |
| subfamily) | | | | | | | ensen) | postSET |
| Suvar4-20 | SET4 | — | H4K20me3 | C. elegans | 288 | 1-288 | 288 | — |
| | | | | | | (Vielle) | | |
| SET8 | SET1 | — | H4K20me1 | C. elegans | 242 | 1-242 | 242 | — |
| | | | | | | (Vielle) | | |
| SET8 | SETD8 | — | H4K20me1 | H. sapiens | 393 | 185-393 | 209 | 256-382: |
| | | | | | | | (Couture) | SET |
| SET8 | TgSET8 | — | H4K20me1/2/3 | T. gondii | 1893 | 1590-1893 | 304 | 1749-1884: |
| | | | | | | (Sautel) | | SET |

Pennini, M. E., Perrinet, S. p., Dautry-Varsat, A. & Subtil, A. Histone methylation by NUE, a novel nuclear effector of the intracellular pathogen Chlamydia trachomatis. PLoS pathogens 6, e1000995 (2010).
Mujtaba, S. et al. Epigenetic transcriptional repression of cellular genes by a viral SET protein. Nature cell biology 10, 1114-1122 (2008).
Tachibana, M., Matsumura, Y., Fukuda, M., Kimura, H. & Shinkai, Y. G9a/GLP complexes independently mediate H3K9 and DNA methylation to silence transcription. The EMBO journal 27, 2681-2690 (2008).
Snowden, A. W., Gregory, P. D., Case, C. C. & Pabo, C. 0. Gene-specific targeting of H3K9 methylation is sufficient for initiating repression in vivo. Current biology 12, 2159-2166 (2002).
Rathert, P., Zhang, X., Freund, C., Cheng, X. & Jeltsch, A. Analysis of the substrate specificity of the Dim-5 histone lysine methyltransferase using peptide arrays. Chemistry & biology 15, 5-11 (2008).
Jackson, J. P. et al. Dimethylation of histone H3 lysine 9 is a critical mark for DNA methylation and gene silencing in Arabidopsis thaliana. Chromosoma 112, 308-315 (2004).
Thorstensen, T. et al. The Arabidopsis SUVR4 protein is a nucleolar histone methyltransferase with preference for monomethylated H3K9. Nucleic acids research 34, 5461-5470 (2006).
Vielle, A. et al. H4K20me1 Contributes to Downregulation of X-Linked Genes for C. elegans Dosage Compensation. PLoS Genetics 8, e1002933 (2012).
Couture, J.-F., Collazo, E., Brunzelle, J. S. & Trievel, R. C. Structural and functional analysis of SET8, a histone H4 Lys-20 methyltransferase. Genes & development 19, 1455-1465 (2005).
Sautel, C. 1. F. et al. SET8-mediated methylations of histone H4 lysine 20 mark silent heterochromatic domains in apicomplexan genomes. Molecular and cellular biology 27, 5711-5724 (20071

TABLE 11
Histone Methyltransferase (HMT) Recruiter Effector Domains

| | | | Modification | | Full | Selected | Final | |
|---|---|---|---|---|---|---|---|---|
| Subtype/ | | Substrate | (if | | size | truncation | size | Catalytic |
| Complex | Name | (if known) | known) | Organism | (aa) | (aa) | (aa) | domain |
| — | Hp1a | — | H3K9me3 | M. musculus | 191 | 73-191 | 119 | 121-179: |
| | | | | | | | (Hathaway) | chromoshadow |
| — | PHF19 | — | H3K27me3 | H. sapiens | 580 | (1-250) + | 335 | 163-250: |
| | | | | | | GGSG | (Ballare) | PHD2 |
| | | | | | | linker | | |
| | | | | | | (SEQ ID | | |
| | | | | | | NO: 131) + | | |
| | | | | | | (500-580) | | |
| — | NIPP1 | — | H3K27me3 | H. sapiens | 351 | 1-329 | 329 | 310-329: |
| | | | | | | (Jin) | | EED |

Hathaway, N. A. et al. Dynamics and memory of heterochromatin in living cells. Cell (2012).
Ballaré, C. et al. Phf19 links methylated Lys36 of histone H3 to regulation of Polycomb activity. Nature structural & molecular biology 19, 1257-1265 (2012).
Jin, Q. et al. The protein phosphatase-1 (PP1) regulator, nuclear inhibitor of PP1 (NIPP1), interacts with the polycomb group protein, embryonic ectoderm development (EED), and functions as a transcriptional repressor. Journal of Biological Chemistry 278, 30677- 30685 (2003).

TABLE 12
Histone Acetyltransferase Inhibitor Effector Domains

| | | Substrate | | | Full | Selected | Final | |
|---|---|---|---|---|---|---|---|---|
| Subtype/ | | (if | Modification | | size | truncation | size | Catalytic |
| Complex | Name | known) | (if known) | Organism | (aa) | (aa) | (aa) | domain |
| — | SET/TAF-1β | — | — | M. musculus | 289 | 1-289 | 289 | — |
| | | | | | | (Cervoni) | | |

Cervoni, N., Detich, N., Seo, S.-B., Chakravarti, D. & Szyf, M. The oncoprotein Set/TAF-1(E≤, an inhibitor of histone acetyltransferase, inhibits active demethylation of DNA, integrating DNA methylation and transcriptional silencing. Journal of Biological Chemistry 277, 25026-25031(2002)

Supplementary Sequences

> TALE(Ngn2)-NLS-CRY2
(SEQ ID NO: 132)
MSRTRLPSPPAPSPAFSADSFSDLLRQFDPSLFNTSLFDSLPPFGAHHTEAATG
EWDEVQSGLRAADAPPPTMRVAVTAARPPRAKPAPRRRAAQPSDASPAAQVDLRTLGY
SQQQQEKIKPKVRSTVAQHHEALVGHGFTHAHIVALSQHPAALGTVAVKYQDMIAALP
EATHEAIVGVGKQWSGARALEALLTVAGELRGPPLQLDTGQLLKIAKRGGVTAVEAVH
AWRNALTGAPLNLTPEQVVAIASNNGGKQALETVQRLLPVLCQAHGLTPEQVVAIASN
GGGKQALETVQRLLPVLCQAHGLTPEQVVAIASNNGGKQALETVQRLLPVLCQAHGLT
PEQVVAIASNIGGKQALETVQRLLPVLCQAHGLTPEQVVAIASNIGGKQALETVQRLLPV
LCQAHGLTPEQVVAIASNGGGKQALETVQRLLPVLCQAHGLTPEQVVAIASNNGGKQA
LETVQRLLPVLCQAHGLTPEQVVAIASNIGGKQALETVQRLLPVLCQAHGLTPEQVVAI
ASNGGGKQALETVQRLLPVLCQAHGLTPEQVVAIASNNGGKQALETVQRLLPVLCQAH
GLTPEQVVAIASNIGGKQALETVQRLLPVLCQAHGLTPEQVVAIASNGGGKQALETVQR
LLPVLCQAHGLTPEQVVAIASNIGGKQALETVQRLLPVLCQAHGLTPEQVVAIASNIGGK
QALETVQRLLPVLCQAHGLTPEQVVAIASNGGGKQALETVQRLLPVLCQAHGLTPEQV
VAIASNIGGKQALETVQRLLPVLCQAHGLTPEQVVAIASHDGGRPALESIVAQLSRPDPA
LAALTNDHLVALACLGGRPALDAVKKGLPHAPALIKRTNRRIPERTSHRVADHAQVVR
VLGFFQCHSHPAQAFDDAMTQFGMSRHGLLQLFRRVGVTELEARSGTLPPASQRWDRI
LQASGMKRAKPSPTSTQTPDQASLHAFADSLERDLDAPSPMHEGDQTRASASPKKKRK
VEASKMDKKTIVWFRRDLRIEDNPALAAAAHEGSVFPVFIWCPEEEGQFYPGRASRWW
MKQSLAHLSQSLKALGSDLTLIKTHNTISAILDCIRVTGATKVVFNHLYDPVSLVRDHTV
KEKLVERGISVQSYNGDLLYEPWEIYCEKGKPFTSFNSYWKKCLDMSIESVMLPPPWRL
MPITAAAEAIWACSIEELGLENEAEKPSNALLTRAWSPGWSNADKLLNEFIEKQLIDYAK
NSKKVVGNSTSLLSPYLHFGEISVRHVFQCARMKQIIWARDKNSEGEESADLFLRGIGLR
EYSRYICFNFPFTHEQSLLSHLRFFPWDADVDKFKAWRQGRTGYPLVDAGMRELWATG
WMHNRIRVIVSSFAVKFLLLPWKWGMKYFWDTLLDADLECDILGWQYISGSIPDGHEL
DRLDNPALQGAKYDPEGEYIRQWLPELARLPTEWIHHPWDAPLTVLKASGVELGTNYA
KPIVDIDTARELLAKAISRTREAQIMIGAAPDEIVADSFEALGANTIKEPGLCPSVSSNDQ
QVPSAVRYNGSKRVKPEEEEERDMKKSRGFDERELFSTAESSSSSSVFFVSQSCSLASEG
KNLEGIQDSSDQITTSLGKNG
> TALE(Ngn2)-NLS-CRY2PHR
(SEQ ID NO: 133)
MSRTRLPSPPAPSPAFSADSFSDLLRQFDPSLFNTSLFDSLPPFGAHHTEAATG
EWDEVQSGLRAADAPPPTMRVAVTAARPPRAKPAPRRRAAQPSDASPAAQVDLRTLGY
SQQQQEKIKPKVRSTVAQHHEALVGHGFTHAHIVALSQHPAALGTVAVKYQDMIAALP
EATHEAIVGVGKQWSGARALEALLTVAGELRGPPLQLDTGQLLKIAKRGGVTAVEAVH
AWRNALTGAPLNLTPEQVVAIASNNGGKQALETVQRLLPVLCQAHGLTPEQVVAIASN
GGGKQALETVQRLLPVLCQAHGLTPEQVVAIASNNGGKQALETVQRLLPVLCQAHGLT
PEQVVAIASNIGGKQALETVQRLLPVLCQAHGLTPEQVVAIASNIGGKQALETVQRLLPV
LCQAHGLTPEQVVAIASNGGGKQALETVQRLLPVLCQAHGLTPEQVVAIASNNGGKQA
LETVQRLLPVLCQAHGLTPEQVVAIASNIGGKQALETVQRLLPVLCQAHGLTPEQVVAI
ASNGGGKQALETVQRLLPVLCQAHGLTPEQVVAIASNNGGKQALETVQRLLPVLCQAH
GLTPEQVVAIASNIGGKQALETVQRLLPVLCQAHGLTPEQVVAIASNGGGKQALETVQR
LLPVLCQAHGLTPEQVVAIASNIGGKQALETVQRLLPVLCQAHGLTPEQVVAIASNIGGK
QALETVQRLLPVLCQAHGLTPEQVVAIASNGGGKQALETVQRLLPVLCQAHGLTPEQV
VAIASNIGGKQALETVQRLLPVLCQAHGLTPEQVVAIASHDGGRPALESIVAQLSRPDPA
LAALTNDHLVALACLGGRPALDAVKKGLPHAPALIKRTNRRIPERTSHRVADHAQVVR
VLGFFQCHSHPAQAFDDAMTQFGMSRHGLLQLFRRVGVTELEARSGTLPPASQRWDRI
LQASGMKRAKPSPTSTQTPDQASLHAFADSLERDLDAPSPMHEGDQTRASASPRKKRK
VEASKMDKKTIVWFRRDLRIEDNPALAAAAHEGSVFPVFIWCPEEEGQFYPGRASRWW
MKQSLAHLSQSLKALGSDLTLIKTHNTISAILDCIRVTGATKVVFNHLYDPVSLVRDHTV
KEKLVERGISVQSYNGDLLYEPWEIYCEKGKPFTSFNSYWKKCLDMSIESVMLPPPWRL
MPITAAAEAIWACSIEELGLENEAEKPSNALLTRAWSPGWSNADKLLNEFIEKQLIDYAK
NSKKVVGNSTSLLSPYLHFGEISVRHVFQCARMKQIIWARDKNSEGEESADLFLRGIGLR
EYSRYICFNFPFTHEQSLLSHLRFFPWDADVDKFKAWRQGRTGYPLVDAGMRELWATG
WMHNRIRVIVSSFAVKFLLLPWKWGMKYFWDTLLDADLECDILGWQYISGSIPDGHEL
DRLDNPALQGAKYDPEGEYIRQWLPELARLPTEWIHHPWDAPLTVLKASGVELGTNYA
KPIVDIDTARELLAKAISRTREAQIMIGAAP
> CIB1-NLS-VP64_2A_GFP
(SEQ ID NO: 134)
MNGAIGGDLLLNFPDMSVLERQRAHLKYLNPTFDSPPLAGFFADSSMITGGEM
DSYLSTAGLNLPMMYGETTVEGDSRLSISPETTLGTGNFKKRKFDTETKDCNEKKKKMT
MNRDDLVEEGEEEKSKITEQNNGSTKSIKKMKHKAKKEENNFSNDSSKVTKELEKTDYI
HVRARRGQATDSHSIAERVRREKISERMKFLQDLVPGCDKITGKAGMLDEIINYVQSLQ
RQIEFLSMLKAIVNPRPDFDMDDIFAKEVASTPMTVVPSPEMVLSGYSHEMVHSGYSSE
MVNSGYLHVNPMQQVNTSSDPLSCFNNGEAPSMWDSHVQNLYGNLGVASPKKKRKV
EASGSGRADALDDFDLDMLGSDALDDFDLDMLGSDALDDFDLDMLGSDALDDFDLDM
LINSRGSGEGRGSLLTCGDVEENPGPVSKGEELFTGVVPILVELDGDVNGHKFSVSGEGE
GDATYGKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALSKDP
NEKRDHMVLLEFVTAAGITLGMDELYK
> CIBN-NLS-VP64_2A_GFP
(SEQ ID NO: 135)
MNGAIGDLLLNFPDMSVLERQRAHLKYLNPTFDSPLAGFFADSSMITGGEM
DSYLSTAGLNLPMMYGETTVEGDSRLSISPETTLGTGNFKKRKFDTETKDCNEKKKKMT
MNRDDLVEEGEEEKSKITEQNNGSTKSIKKMKHKAKKEENNFSNDSSKVTKELEKTDYI
ASPKKKRKVEASGSGRADALDDFDLDMLGSDALDDFDLDMLGSDALDDFDLDMLGSD
ALDDFDLDMLINSRGSGEGRGSLLTCGDVEENPGPVSKGEELFTGVVPILVELDGDVNG
HKFSVSGEGEGDATYGKLTLKFICTTGKLFVPWPTLVTTLTYGVQCFSRYPDHMKQHDF
FKSAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNHY
LSTQSALSKDPNEKRDHMVLLEFVTAAGITLGMDELYK
> CIB1-NLS-VP16_2A_GFP
(SEQ ID NO: 136)
MNGAIGGDLLLNFPDMSVLERQRAHLKYLNPTFDSPLAGFFADSSMITGGEM
DSYLSTAGLNLPMMYGETTVEGDSRLSISPETTLGTGNFKKRKFDTETKDCNEKKKKMT
MNRDDLVEEGEEEKSKITEQNNGSTKSIKKMKHKAKKEENNFSNDSSKVTKELEKTDYI
HVRARRGQATDSHSIAERVRREKISERMKFLQDLVPGCDKITGKAGMLDEIINYVQSLQ
RQIEFLSMKLAIVNPRPDFDMDDIFAKEVASTPMTVVPSPEMVLSGYSHEMVHSGYSSE
MVNSGYLHVNPMQQVNTSSDPLSCFNNGEAPSMWDSHVQNLYGNLGVASPKKKRKY
EASAPPTDVSLGDELHLDGEDVAMAHADALDDFDLDMLGDGDSPGPGFTPHDSAPYG
ALDMADFEFEQMFTDALGIDEYGGEFPGIRRSRGSGEGRGSLLTCGDVEENPGPVSKGE
ELFTGVVPILVELDGDVNGHKFSVSGEGEGDATYGKLTLKFICTTGKLPVPWPTLVTTLT
YGVQCFSRYPDHMKQHDFFKSAMPEGYVQERTIFFKDDGNYKTRAEVKFEGDTLVNRI
ELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYK
> CIB1-NLS-P65_2A_GFP
(SEQ ID NO: 137)
MNGAIGGDLLLNFPDMSVLERQRAHLKYLNPTFDSPLAGFFADSSMITGGEM
DSYLSTAGLNLPMMYGETTVEGDSRLSISPETTLGTGNFKKRKFDTETKDCNEKKKKMT
MNRDDLVEEGEEEKSKITEQNNGSTKSIKKMKHKAKKEENNFSNDSSKVTKELEKTDYI
HVRARRGQATDSHSIAERVRREKISERMKFLQDLVPGCDKITGKAGMLDEIINYVQSLQ
RQIEFLSMKLAIVNPRPDFDMDDIFAKEVASTPMTVVPSPEMVLSGYSHEMVHSGYSSE
MVNSGYLHVNPMQQVNTSSDPLSCFNNGEAPSMWDSHVQNLYGNLGVASPKKKRKV
EASPSGQISNQALALAPSSAPVLAQTMVPSSAMVPLAQPPAPAPVLTPGPPQSLSAPVPK
STQAGEGTLSEALLHLQFDADEDLGALLGNSTDPGVFTDLASVDNSEFQQLLNQGVSMS
HSTAEPMLMEYPEAITRLVTGSQRPPDPAPTPLGTSGLPNGLSGDEDFSSIADMDFSALLS
QISSSGQSRGSGEGRGSLLTCGDVEENPGPVSKGEELFTGVVPILVELDGDVNGHKFSVS
GEGEGDATYGKLTLKFICTTGKLPVPWPTLVTTLTYGVQCFSRYPDHMKQHDFFKSAM
PEGYVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLSTQSA
LSKDPNEKRDHMVLLEFVTAAGITLGMDELYK
.> HA-TALE(12mer)-NLS-VP64_2A_GFP
(SEQ ID NO: 138)
MYPYDVPDYAVDLRTLGYSQQQQEKIKPKVRSTVAQHHEALVGHGFTHAHI
VALSQHPAALGTVAVKYQDMIAALPEATHEAIVGVGKQWSGARALEALLTVAGELRGP
PLQLDTGQLLKIAKRGGVTAVEAVHAWRNALTGAPLNLTPEQVVAIASXXGGKQALET
VQRLLPVLCQAHGLTPEQVVAIASXXGGKQALETVQRLLPVLCQAHGLTPEQVVAIASX
XGGKQALETVQRLLPVLCQAHGLTPEQVVAIASXXGGKQALETVQRLLPVLCQAHGLT
PEQVVAIASXXGGKQALETVQRLLPVLCQAHGLTPEQVVAIASXXGGKQALETVQRLLP
VLCQAHGLTPEQVVAIASXXGGKQALETVQRLLPVLCQAHGLTPEQVVAIASXXGGKQ
ALETVQRLLPVLCQAHGLTPEQVVAIASXXGGKQALETVQRLLPVLCQAHGLTPEQVV
AIASXXGGKQALETVQRLLPVLCQAHGLTPEQVVAIASXXGGKQALETVQRLLPVLCQ
AHGLTPEQVVAIASXXGGKQALETVQRLLPVLCQAHGLTPEQVVAIASXXGGRPALESI
VAQLSRPDPALAALTNDHLVALACLGGRPALDAVKKGLPHAPALIKRTNRRIPERTSHR
VAASPKKKRKVEASGSGRADALDDFDLDMLGSDALDDFDLDMLGSDALDDFDLDMLG
SDALDDFDLDMLINSRGSGEGRGSLLTCGDVEENPGPVSKGEELFTGVVPILVELDGDV
NGHKFSVSGEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDN
HYLSTQSALSKDPNEKRDHMVLLEFVTAAGITLGMDELYK
> HA-TALE(12mer)-NLS-SID4X_2A_phiLOV2.1
(SEQ ID NO: 139)
MYPYDVPDYAVDLRTLGYSQQQQEKIKPKVRSTVAQHHEALVGHGFTHAHI
VALSQHPAALGTVAVKYQDMIAALPEATHEAIVGVGKQWSGARALEALLTVAGELRGP
PLQLDTGQLLKIAKRGGVTAVEAVHAWRNALTGAPLNLTPEQVVAIASXXGGKQALET
VQRLLPVLCQAHGLTPEQVVAIASXXGGKQALETVQRLLPVLCQAHGLTPEQVVAIASX
XGGKQALETVQRLLPVLCQAHGLTPEQVVAIASXXGGKQALETVQRLLPVLCQAHGLT
PEQVVAIASXXGGKQALETVQRLLPVLCQAHGLTPEQVVAIASXXGGKQALETVQRLLP
VLCQAHGLTPEQVVAIASXXGGKQALETVQRLLPVLCQAHGLTPEQVVAIASXXGGKQ
ALETVQRLLPVLCQAHGLTPEQVVAIASXXGGKQALETVQRLLPVLCQAHGLTPEQVV
AIASXXGGKQALETVQRLLPVLCQAHGLTPEQVVAIASXXGGKQALETVQRLLPVLCQ
AHGLTPEQVVAIASXXGGKQALETVQRLLPVLCQAHGLTPEQVVAIASXXGGRPALESI
VAQLSRPDPALAALTNDHLVALACLGGRPALDAVKKGLPHAPALIKRTNRRIPERTSHR
VAASPKKKRKVEASPKKKRKVHASGSGMNIQMLLEAADYLERREREAEHGYASMLPG
SGMNIQMLLEAADYLERREREAEHGYASMLPGSGMKIQMLLEAADYLBRREREAEHG
YASMLPGSGMNIQMLLEAADYLERREREAEHGYASMLPSRSRGSGEGRGSLLTCGDVE
ENPGPIEKSFVITDPRLPDYPIIFASDGFLELTEYSREEIMGRNARFLQGPETDQATVQKIR
DAIRDQRETTVQLINYTKSGKKFWNLLHLQPVRDRKGGLQYFIGVQLVGSDHV
> HA-TALE(12mer)-NLS-CIB1
(SEQ ID NO: 140)
MYPYDVPDYAVDLRTLGYSQQQQEKIKPKVRSTVAQHHEALVGHGFTHAHI
VALSQHPAALGTVAVKYQDMIAALPEATHEAIVGVGKQWSGARALEALLTVAGELRGP
PLQLDTGQLLKIAKRGGVTAVEAVHAWRNALTGAPLNLTPEQVVAIASXXGGKQALET
VQRLLPVLCQAHGLTPEQVVAIASXXGGKQALETVQRLLPVLCQAHGLTPEQVVAIASX
XGGKQALETVQRLLPVLCQAHGLTPEQVVAIASXXGGKQALETVQRLLPVLCQAHGLT
PEQVVAIASXXGGKQALETVQRLLPVLCQAHGLTPEQVVAIASXXGGKQALETVQRLLP
VLCQAHGLTPEQVVAIASXXGGKQALETVQRLLPVLCQAHGLTPEQVVAIASXXGGKQ
ALETVQRLLPVLCQAHGLTPEQVVAIASXXGGKQALETVQRLLPVLCQAHGLTPEQVV
AIASXXGGKQALETVQRLLPVLCQAHGLTPEQVVAIASXXGGKQALETVQRLLPVLCQ
AHGLTPEQVVAIASXXGGKQALETVQRLLPVLCQAHGLTPEQVVAIASXXGGRPALESI
VAQLSRPDPALAALTNDHLVALACLGGRPALDAVKKGLPHAPALIKRTNRRIPERTSHR
VAASPKKKRKVEASNGAIGGDLLLNFPDMSVLERQRAHLKYLNPTFDSPLAGFFADSSM
ITGGEMDSYLSTAGLNLPMMYGETTVEGDSRLSISPETTLGTGNFKKRKFDTETKDCNE
KKKKMTMNRDDLVEEGEEEKSKITEQNNGSTKSIKKMKHKAKKEENNFSNDSSKVTKE
LEKTDYIHVRARRGQATDSHSIAERVRRKKISERMKFLQDLVPGCDKITGKAGMLDEIIN
YVQSLQRQIEFLSMKLAIVNPRPDFDMDDIFAKEVASTPMTVVPSPEMVLSGYSHEMVH
SGYSSEMVNSGYLHVNPMQQVNTSSDPLSCFNNGEAPSMWDSHVQNLYGNLGV
> CRY2PHR-NLS-VP64_2A_GFP
(SEQ ID NO: 141)
MKMDKKTIVWFRRDLRIEDNPALAAAAHEGSVFPVFIWCPEEEGQFYPGRAS
RWWMKQSLAHLSQSLKALGSDLTLIKTHNTISAILDCIRVTGATKVVFNHLYDPVSLVR
DHTVKEKLVERGISVQSYNGDLLYEPWEIYCEKGKPFTSFNSYWKKCLDMSIESVMLPP
PWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLR
GIGLREYSRYICFNFPFTHEQSLLSHLRFFPWDADVDKFKAWRQGRTGYPLVDAGMREL
WATGWMHNRIRVIVSSFAVKLLLPWKWGMKYFWDTLLDADLECDILGWQYISGSIPD
GHELDRLDNPALQGAKYDPEGEYIRQWLPELARLPTEWIHHPWDAPLTVLKASGVELG
TNYAKPIVDIDTARELLAKAISRTREAQIMIGAAPASPKKKRKVEASGSGRADALDDFDL
DMLGSDALDDFDLDMLGSDALDDFDLDMLGSDALDDFDLDMLINSRGSGEGRGSLLTC
GDVEENPGPVSKGEELFTGVVPILVELDGDVNGHKFSVSGEGEGDATYGKLTLKFICTT
GKLPVPWPTLVTTLTYGVQCFSRYPDHMKQHDFFKSAMPEGYVQERTIFFKDDGNYKT
RAEVKFEGDTLVNRIELKGIDFKEDGNILGHKLEYNYNSHNVYIMADKQKNGIKVNFKI
RHNIEDGSVQLADHYQQNTPIGDGPVLLPDNHYLSTQSALSKDPNEKRDHMVLLEFVTA
AGITLGMDELYKV
> CRY2PHR-NLS-SID4X_2A_phiLOV2.1
(SEQ ID NO: 142)
MKMDKKTIVWFRRDLRIEDNPALAAAAHEGSVFPVFIWCPEEEGQFVPGRAS
RWWMKQSLAHLSQSLKALGSDLTLIKTHNTISAILDCIRVTGATKVVFNHLYDPVSLVR
DHTVKEKLVERGISVQSYNGDLLYEPWEIYCEKGKPFTSFNSYWKKCLDMSIESVMLPP
PWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQHWARDKNSEGEESADLFLR
GIGLREYSRYICFNFPFTHEQSLLSHLRFFPWDADVDKFKAWRQGRTGYPLVDAGMREL
WATGWMHNRIRVIVSSFAVKFLLLPWKWGMKYFWDTLLDADLECDILGWQYISGSIPD
GHELDRLDNPALQGAKYDPEGEYIRQWLPELARLPTEWIHHPWDAPLTVLKASGVELG
TNYAKPIVDIDTARELLAKAISRTREAQIMIGAAPASPKKKRKVEASGSGMNIQMLLEAA
DYLERREREAEHGYASMLPGSGMNIQMLLEAADYLERREREAEHGYASMLPGSGMNIQ
MLLEAADYLERREREAEHGYASMLPGSGMNIQMLLEAADYLERREREAEHGYASMLP
SRSRGSGEGRGSLLTCGDVEENPGPIEKSFVITDPRLPDYPIIFASDGFLELTEYSREEIMG
RNARFLQGPETDQATVQKIRDAIRDQRETTVQLINYTKSGKKFWNLLHLQPVRDRKGGL
QYFIGVQLVGSDHV
> TALE(KLF4)-NLS-CRY2PHR
(SEQ ID NO: 143)
MSRTRLPSPPAPSPAFSADSFSDLLRQFDPSLFNTSLFDSLPPFGAHHTEAATG
EWDEVQSGLRAADAPPPTMRVAVTAARPPRAKPAPRRRAAQPSDASPAAQVDLRTLGY
SQQQQEKIKPKVRSTVAQHHEALVGHGFTHAHIVALSQHPAALGTVAVKYQDMIAALP
EATHEAIVGVGKQWSGARALEALLTVAGELRGPPLQLDTGQLLKIAKRGGVTAVEAVH
AWRNALTGAPLNLTPEQVVAIASNGGGKQALETVQRLLPVLCQAHGLTPEQVVAIASH
DGGKQALETVQRLLPVLCQAHGLTPEQVVAIASNGGGKQALETVQRLLPVLCQAHGLT
PEQVVAIASNGGGKQALETVQRLLPVLCQAHGLTPEQVVAIASNIGGKQALETVQRLLP
VLCQAHGLTPEQVVAIASHDGGKQALETVQRLLPVLCQAHGLTPEQVVAIASNGGGKQ
ALETVQRLLPVLCQAHGLTPEQVVAIASNGGGKQALETVQRLLPVLCQAHGLTPEQVV
AIASNIGGKQALETVQRLLPVLCQAHGLTPEQVVAIASNGGGKQALETVQRLLPVLCQA
HGLTPEQVVAIASNIGGKQALETVQRLLPVLCQAHGLTPEQVVAIASNIGGKQALETVQ
RLLPVLCQAHGLTPEQVVAIASHDGGRPALESIVAQLSRPDPALAALTNDHLVALACLG
GRPALDAVKKGLPHAPALIKRTNRRIPERTSHRVADHAQVVRVLGFFQCHSHPAQAFDD
AMTQFGMSRHGLLQLFRRVGVTELEARSGTLPPASQRWDRILQASGMKRAKPSPTSTQ
TPDQASLHAFADSLERDLDAPSPMHEGDQTRASASPKKKRKVEASKMDKKTVWFRRD
LRIEDNPALAAAAHEGSVFPVFIWCPEEEGQFYPGRASRWWMKQSLAHLSQSLKALGS
DLTLIKTHNTISAILDCIRVTGATKVVFNHLYDPVSLVRDHTVKEKLVERGISVQSYNGD
LLYEPWEIYCEKGKPFTSFNSYWKKCLDMSIESVMLPPPWRLMPITAAAEAIWACSIEEL
GLENEAEKPSNALLTRAWSPGWSNADKLLNEFIEKQLIDYAKNSKKVVGNSTSLLSPYL
HFGEISVRHVFQCARMKQIIWARDKNSEGEESADLFLRGIGLREYSRYICFNFPFTHEQSL
LSHLRFFPWDADVDKFKAWRQGRTGYPLVDAGMRELWATGWMHNRIRVIVSSFAVKF
LLLPWKWGMKYFWDTLLDADLECDILGWQYISGSIPDGHELDRLDNPALQGAKYDPEG
EYIRQWLPELARLPTEWIHHPWDAPLTVLKASGVELGTNYAKPIVDIDTARELLAKAISR
TREAQIMIGAAP
> HA-NLS-TALE(p11, N136)-SID
(SEQ ID NO: 144)
MYPYDVPDYASPKKKRKVEASVDLRTLGYSQQQQEKIKPKVRSTVAQHHEA
LVGHGFTHAHIVALSQHPAALGTVAVKYQDMIAALPEATHEAIVGVGKQWSGARALEA
LLTVAGELRGPPLQLDTGQLLKIAKRGGVTAVEAVHAWRNALTGAPLNLTPEQVVAIA
SNNGGKQALETVQRLLPVLCQAHGLTPEQVVAIASHDGGKQALETVQRLLPVLCQAHG
LTPEQVVAIASHDGGKQALETVQRLLPVLCQAHGLTPEQVVAIASNGGKQALETVQR
LLPVLCQAHGLTPEQVVAIASNNGQKQALETVQRLLPVLCQAHGLTPEQVVAIASHDGG
KQALETVQRLLPVLCQAHGLTPEQVVAIASMDGGHQALETVQRLLPVLCQAHGLTPEQ
VVAIASHDGGKQALETVQRRLPVLCQAHGLTPEQVVAIASNGGGKQALETVQRLLPVL
CQAHGLTPEQVVAIASHDGGKQALETVQRLLPVLCQAHGLTPEQVVAIASHDGGKQAL
ETVQRLLPVLCQAHGLTPEQVVAIASNIGGKQALETVQRLLPVLCQAHGLTPEQVVAIA
SNNGGKQALETVQRLLPVLCQAHGLTPEQVVAIASNNGGKQALETVQRLLPVLCQAHG
LTPEQVVAIASHDGGKQALETVQRLLPVLCQAHGLTPEQVVAIASNGGGKQALETVQR
LLPVLCQAHGLTPEQVVAIASHDGGKQALETVQRLLPVLCQAHGLTPEQVVAIASHDGG
KQALETVQRLLPVLCQAHGLTPEQVVAIASHDGGRPALESIVAQLSRPDPALAALTNDH
LVALACLGGRPALDAVKKGLPHAPALIKRTNRRIPERTSHRVADHAQVVRVLGFFQCHS
HPAQAFDDAMTQFGMSRHGLLQLFRRVGVTELEARSGTLPPASQRWDRILQASGMKRA
KPSPTSTQTPDQASLHAFADSLERDLDAPSPMHEGDQTRASASGSGMNIQMLLEAADYL
ERREREAEHGYASMLP.
> HA-NLS-TALE(p11, N136)-SID4X
(SEQ ID NO: 145)
MYPYDVPDYASPKKKRKVEASVDLRTLGYSQQQQEKIKPKVRSTVAQHHEA
LVGHGFTHAHIVALSQHPAALGTVAVKYQDMIAALPEATHEAIVGVGKQWSGARALEA
LLTVAGELRGPPLQLDTGQLLKIAKRGGVTAVEAVHAWRNALTGAPLNLTPEQVVAIA
SNNGGKQALETVQRLLPVLCQAHGLTPEQVVAIASHDGGKQALETVQRLLPVLCQAHG
LTPEQVVAIASHDGGKQALETVQRLLPVLCQAHGLTPEQVVAIASNGGKQALETVQR
LLPVLCQAHGLTPEQVVAIASNNGQKQALETVQRLLPVLCQAHGLTPEQVVAIASHDGG
KQALETVQRLLPVLCQAHGLTPEQVVAIASMDGGHQALETVQRLLPVLCQAHGLTPEQ
VVAIASHDGGKQALETVQRRLPVLCQAHGLTPEQVVAIASNGGGKQALETVQRLLPVL
CQAHGLTPEQVVAIASHDGGKQALETVQRLLPVLCQAHGLTPEQVVAIASHDGGKQAL
ETVQRLLPVLCQAHGLTPEQVVAIASNIGGKQALETVQRLLPVLCQAHGLTPEQVVAIA
SNNGGKQALETVQRLLPVLCQAHGLTPEQVVAIASNNGGKQALETVQRLLPVLCQAHG
LTPEQVVAIASHDGGKQALETVQRLLPVLCQAHGLTPEQVVAIASNGGGKQALETVQR
LLPVLCQAHGLTPEQVVAIASHDGGKQALETVQRLLPVLCQAHGLTPEQVVAIASHDGG
KQALETVQRLLPVLCQAHGLTPEQVVAIASHDGGRPALESIVAQLSRPDPALAALTNDH
LVALACLGGRPALDAVKKGLPHAPALIKRTNRRIPERTSHRVADHAQVVRVLGFFQCHS
HPAQAFDDAMTQFGMSRHGLLQLFRRVGVTELEARSGTLPPASQRWDRILQASGMKRA
KPSPTSTQTPDQASLHAFADSLERDLDAPSPMHEGDQTRASASGSGMNIQMLLEAADYL
ERREREAEHGYASMLPGSGMNIQMLLEAADYLERREREAEHGYASMLPGSGMNIQML
LEAADYLERREREAEHGYASMLPGSGMNIQMLLEAADYLERREREAEHGYASMLPSR
>HA-TALE(ng2, C63)-GS-cib1-mutNLS
(SEQ ID NO: 146)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
TPMTVVPSPEMVLSGYSHEMVHSGYSSEMVNSGYLHVNPMQQVNTSSDPLSCFNNGEA
PSMWDSHVQNLYGNLGV
> HA-TALE(ng2, C63)-wNES-cib1-mutNLS
(SEQ ID NO: 147)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLYPERLRRILTNGAIGGDLLLNFPDMSVLERQRA
HLKYLNPTFDSPLAGFFADSSMITGGEMDSYLSTAGLNLPMMYGETTVEGDSRLSISPET
TLGTGNFKAAKFDTETKDCNEAAKKMTMNRDDLVEEGEEEKSKITEQNNGSTKSIKKM
KHKAKKEENNFSNDSSKVTKELEKTDYIHVRARRGQATDSHSIAERVRREKISERMKFL
QDLVPGCDKITGKAGMLDEIINYVQSLQRQIEFLSMKLAIVNPRPDFDMDDIFAKEVAST
PMTVVPSPEMVLSGYSHEMVHSGYSSEMVNSGYLHVNPMQQVNTSSDPLSCFNNGEAP
SMWDSHVQNLYGNLGV
> HA-TALE(ng2, C63)-mNES-cib1-mutNLS
(SEQ ID NO: 148)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLQLPPLERLTLNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
TPMTVVPSPEMVLSGYSHEMVHSGYSSEMVNSGYLHVNPMQQVNTSSDPLSCFNNGEA
PSMWDSHVQNLYGNLGV
> HA-TALE(ng2, C63)-ptk2NES-cib1-mutNLS
(SEQ ID NO: 149)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLDLASLILNGAIGGDLLLNFPDMSVLERQRAHL
KYLNPTFDSPLAGFFADSSMITGGEMDSYLSTAGLNLPMMYGETTVEGDSRLSISPETTL
GTGNFKAAKFDTETKDCNEAAKKMTMNRDDLVEEGEEEKSKITEQNNGSTKSIKKMKH
KAKKEENNFSNDSSKVTKELEKTDYIHVRARRGQATDSHSIAERVRREKISERMKFLQD
LVPGCDKITGKAGMLDEIINYVQSLQRQIEFLSMKLAIVNPRPDFDMDDIFAKEVASTPM
TVVPSPEMVLSGYSHEMVHSGYSSEMVNSGYLHVNPMQQVNTSSDPLSCFNNGEAPSM
WDSHVQNLYGNLGV
> HA-TALE(ng2, C63)-mapkkNES-cib1-mutNLS
(SEQ ID NO: 150)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLQKKLEELELNGAIGGDLLLNFPDMSVLERQRA
HLKYLNPTFDSPLAGFFADSSMITGGEMDSYLSTAGLNLPMMYGETTVEGDSRLSISPET
TLGTGNFKAAKFDTETKDCNEAAKKMTMNRDDLVEEGEEEKSKITEQNNGSTKSIKKM
KHKAKKEENNFSNDSSKVTKELEKTDYIHVRARRGQATDSHSIAERVRREKISERMKFL
QDLVPGCDKITGKAGMLDEIINYVQSLQRQIEFLSMKLAIVNPRPDFDMDDIFAKEVAST
PMTVVPSPEMVLSGYSHEMVHSGYSSEMVNSGYLHVNPMQQVNTSSDPLSCFNNGEAP
SMWDSHVQNLYGNLGV
> HA-TALE(ng2, C63)-GS-cib1Δ3-mutNLS
(SEQ ID NO: 151)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
TPMTVVPSPEMVLSGYSHEMVHSGYSSEMVNSGYLHVNPMQQVNTSS
> HA-TALE(ng2, C63)-wNLS-cib1Δ3-mutNLS
(SEQ ID NO: 152)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
TPMTVVPSPEMVLSGYSHEMVHSGYSSEMVNSGYLHVNPMQQVNTSS
> HA-TALE(ng2, C63)-wNLS-cib1Δ3-mutNLS
(SEQ ID NO: 152)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLYPERLRRILTNGAIGGDLLLNFPDMSVLERQRA
HLKYLNPTFDSPLAGFFADSSMITGGEMDSYLSTAGLNLPMMYGETTVEGDSRLSISPET
TLGTGNFKAAKFDTETKDCNEAAKKMTMNRDDLVEEGEEEKSKITEQNNGSTKSIKKM
KHKAKKEENNFSNDSSKVTKELEKTDYIHVRARRGQATDSHSIAERVRREKISERMKFL
QDLVPGCDKITGKAGMLDEIINYVQSLQRQIEFLSMKLAIVNPRPDFDMDDIFAKEVAST
PMTVVPSPEMVLSGYSHEMVHSGYSSEMVNSGYLHVNPMQQVNTSS
> HA-TALE(ng2, C63)-mNLS-cib1Δ3-mutNLS
(SEQ ID NO: 153)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASSPKKKRKVEASNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
TPMTVVPSPEMVLSGYSHEMVHSGYSSEMVNSGYLHVNPMQQVNTSS
> HA-TALE(ng2, C63)-GS-cib1Δ3-mutNLS-mutbHLH
(SEQ ID NO: 154)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARAAQATDSHSIAEAVAREKISERMK
FLQDLVPGCDKITGKAGMLDEIINYVQSLQRQIEFLSMKLAIVNPRPDFDMDDIFAKEVA
STPMTVVPSPEMVLSGYSHEMVHSGYSSEMVNSGYLHVNPMQQVNTSSDPLSCFNNGE
APSMWDSHVQNLYGNLGV
> HA-TALE(ng2, C63)-wNLS-cib1Δ3-mutNLS-mutbHLH
(SEQ ID NO: 155)
SRTRLPSPPAPSPAFSADSFSDLLRQFDPSLFNTSLFDSLPPFGAHHTEAATGE
WDEVQSGLRAADAPPPTMRVAVTAARPPRAKPAPRRRAAQPSDASPAAQVDLRTLGYS
QQQQEKIKPKVRSTVAQHHEALVGHGFTHAHIVALSQHPAALGTVAVKYQDMIAALPE
ATHEAIVGVGKQWSGARALEALLTVAGELRGPPLQLDTGQLLKIAKRGGVTAVEAVHA
WRNALTGAPLNLTPEQVVAIASNGGGKQALETVQRLLPVLCQAHGLTPEQVVAIASNN
GGKQALETVQRLLPVLCQAHGLTPEQVVAIASNIGGKQALETVQRLLPVLCQAHGLTPE
QVVAIASNIGGKQALETVQRLLPVLCQAHGLTPEQVVAIASNGGGKQALETVQRLLPVL
CQAHGLTPEQVVAIASNNGGKQALETVQRLLPVLCQAHGLTPEQVVAIASNIGGKQALE
TVQRLLPVLCQAHGLTPEQVVAIASNGGGKQALETVQRLLPVLCQAHGLTPEQVVAIAS
NGGGKQALETVQRLLPVLCQAHGLTPEQVVAIASNGGGKQALETVQRLLPVLCQAHGL
TPEQVVAIASNGGGKQALETVQRLLPVLCQAHGLTPEQVVAIASNGGGKQALETVQRLL
PVLCQAHGLTPEQVVAIASNGGGKQALETVQRLLPVLCQAHGLTPEQVVAIASNGGGKQ
ALETVQRLLPVLCQAHGLTPEQVVAIASNGGGKQALETVQRLLPVLCQAHGLTPEQVVA
IASHDGGRPALESIVAQLSRPDPALAALTNDHLVALACLGGRPALDAVKKGLPHAPALI
KRTNRRIPERTSHRVAASLYPERLRRILTNGAIGGDLLLNFPDMSVLERQRAHLKYLNPT
FDSPLAGFFADSSMITGGEMDSYLSTAGLNLPMMYGETTVEGDSRLSISPETTLGTGNFK
AAKFDTETKDCNEAAKKMTMNRDDLVEEGEEEKSKITEQNNGSTKSIKKMKHKAKKE
ENNFSNDSSKVTKELEKTDYIHVRARAAQATDSHSIAEAVAREKISERMKFLQDLVPGC
DKITGKAGMLDEIINYVQSLQRQIEFLSMKLAIVNPRPDFDMDDIFAKEVASTPMTVVPS
PEMVLSGYSHEMVHSGYSSEMVNSGYLHVNPMQQVNTSSDPLSCFNNGEAPSMWDSH
VQNLYGNLGV
> HA-TALE(ng2, C63)-GS-cib1Δ1-mutNLS
(SEQ ID NO: 156)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRGGSVASTPMTVVPSPEMVLSGYSHEMVHS
GYSSEMVNSGYLHVNPMQQVNTSSDPLSCFNNGEAPSMWDSHVQNLYGNLGV
> HA-TALE(ng2, C63)-wNLS-cib1Δ1-mutNLS
(SEQ ID NO: 157)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLYPERLRRILTNGAIGGDLLLNFPDMSVLERQRA
HLKYLNPTFDSPLAGFFADSSMITGGEMDSYLSTAGLNLPMMYGETTVEGDSRLSISPET
TLGTGNFKAAKFDTETKDCNEAAKKMTMNRDDLVEEGEEEKSKITEQNNGSTKSIKKM
KHKAKKEENNFSNDSSKVTKELEKTDYIHVRARRGQATDSHSIAERVRREKISERMKFL
QDLVPGCDKITGKAGMLDEIINYVQSLQRGGSGEEEKSKITEQNNGSTKSIKKMKHKAK
KEENNFSNDSSKVTKELEKTDYIHVRARRGQATDSHSIAERVRREKISERMKFLQDLVP
GCDKITGKAGMLDEIINYVQSLQRGGSVASTPMTVVPSPEMVLSGYSHEMVHSGYSSE
MVNSGYLHVNPMQQVNTSSDPLSCFNNGEAPSMWDSHVQNLYGNLGV
> HA-TALE(ng2, C63)-GS-cib1Δ2-mutNLS
(SEQ ID NO: 158)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
GYSSEMVNSGYLHVNPMQQVNTSSDPLSCFNNGEAPSMWDSHVQNLYGNLGV
> HA-TALE(ng2, C63)-wNLS-cib1Δ2-mutNLS
(SEQ ID NO: 159)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLYPERLRRILTNGAIGGDLLLNFPDMSVLERQRA
HLKYLNPTFDSPLAGFFADSSMITGGEMDSYLSTAGLNLPMMYGETTVEGDSRLSISPET
TLGTGNFKAAKFDTETKDCNEAAKKMTMNRDDLVEEGEEEKSKITEQNNGSTKSIKKM
KHKAKKEENNFSNDSSKVTKELEKTDYIHVRARRGQATDSHSIAERVRREKISERMKFL
QDLVPGCDKITGKAGMLDEIINYVQSLQRQIEFLSMKLAIVNPRPDFDMDDIFAKEVAST
PMTVVPSPEMVLSGYGGSPLSCFNNGEAPSMWDSHVQNLYGNLGV
> HA-TALE(ng2, C63)-NLS-cib1-mutNLS-mutbHLH
(SEQ ID NO: 160)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASSPKKKRKVEASNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARAAQATDSHSIAEAVAREKISERMK
FLQDLVPGCDKITGKAGMLDEIINYVQSLQRQIEFLSMKLAIVNPRPDFDMDDIFAKEVA
STMPTVVPSPEMVLSGYSHEMVHSGYSSEMVNSGYLHVNPMQQVNTSSDPLSCFNNGE
APSMWDSHVQNLYGNLGV
> HA-TALE(ng2, C63)-NLS-cib1Δ1-mutNLS
(SEQ ID NO: 161)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASSPKKKRKVEASNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTKELEKTDYIHVRARRGQATDSHSIAEAVAREKISERMKF
LQDLVPGCDKITGKAGMLDEIINYVQSLQRGGSVASTPMTVVPSPEMVLSGYSHEMVHS
GYSHEMVNSGYLHVNPMQQVNTSSDPLSCFNNGEAPSMWDSHVQNLYGNLGV
> HA-TALE(ng2, C63)-NLS-cib1Δ2-mutNLS
(SEQ ID NO: 162)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASLYPERLRRILTNGAIGGDLLLNFPDMSVLERQRA
HLKYLNPTFDSPLAGFFADSSMITGGEMDSYLSTAGLNLPMMYGETTVEGDSRLSISPET
TLGTGNFKAAKFDTETKDCNEAAKKMTMNRDDLVEEGEEEKSKITEQNNGSTKSIKKM
KHKAKKEENNFSNDSSKVTKELEKTDYIHVRARRGQATDSHSIAEAVAREKISERMKFL
QDLVPGCDKITGKAGMLDEIINYVQSLQRGGSVASTPMTVVPSPEMVLSGYSHEMVHST
PMTVVPSPEMVLGYLGGSPLSCFNNGEAPSMWDSHVQNLYGNLGV
> HA-TALE(ng2, C63)-GS-iNES1-cib1-mutNLS
(SEQ ID NO: 163)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLLYPERLRRILTNPTFDSPLAGFFADSSMITGGEMDSYLSTAGLNLPMMYGETT
VEGDSRLSISPETTLGTGNFKAAKFDTETKDCNEAAKKMTMNRDDLVEEGEEEKSKITE
QNNGSTKSIKKMKHKAKKEENNFSNDSSKVTKELEKTDYIHVRARRGQATDSHSIAERV
RREKISERMKFLQDLVPGCDKITGKAGMLDEIINYVQSLQRQIEFLSMKLAIVNPRPDFD
MDDIFAKEVASTPMTVVPSPEMVLSGYSHEMVHSGYSSEMVNSGYLHVNPMQQVNTS
SDPLSCFNNGEAPSMWDSHVQNLYGNLGV
> HA-TALE(ng2, C63)-GS-iNES2-cib1-mutNLS
(SEQ ID NO: 164)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLLYPTFDSPLAGFFADSSMITGGEMDLYPERLRRILTSYSTAGLNLPMMYGETT
VEGDSRLSISPETTLGTGNFKAAKFDTETKDCNEAAKKMTMNRDDLVEEGEEEKSKITE
QNNGSTKSIKKMKHKAKKEENNFSNDSSKVTKELEKTDYIHVRARRGQATDSHSIAERV
RREKISERMKFLQDLVPGCDKITGKAGMLDEIINYVQSLQRQIEFLSMKLAIVNPRPDFD
MDDIFAKEVASTPMTVVPSPEMVLSGYSHEMVHSGYSSEMVNSGYLHVNPMQQVNTS
SDPLSCFNNGEAPSMWDSHVQNLYGNLGV
> HA-TALE(ng2, C63)-GS-iNES3-cib1-mutNLS
(SEQ ID NO: 165)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGLYPERLRR
ILTDSRLSISPETTLGTGNFKAAKFDTETKDCNEAAKKMTMNRDDLVEEGEEEKSKITEQ
NNGSTKSIKKMKHKAKKEENNFSNDSSKVTKELEKTDYIHVRARRGQATDSHSIAERVR
REKISERMKFLQDLVPGCDKITGKAGMLDEIINYVQSLQRQIEFLSMKLAIVNPRPDFDM
DDIFAKEVASTPMTVVPSPEMVLSGYSHEMVHSGYSSEMVNSGYLHVNPMQQVNTSSD
PLSCFNNGEAPSMWDSHVQNLYGNLGV
> HA-TALE(ng2, C63)-GS-iNES4-cib1-mutNLS
(SEQ ID NO: 166)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAAAKKMTMNRDDLVEEGLYPERLRRILTEEEKSKIT
EQNNGSTKSIKKMKHKAKKEENNFSNDSSKVTKELEKTDYIHVRARRGQATDSHSIAER
VRREKISERMKFLQDLVPGCDKITGKAGMLDEIINYVQSLQRQIEFLSMKLAIVNPRPDF
DMDDIFAKEVASTPMTVVPSPEMVLSGYSHEMVHSGYSSEMVNSGYLHVNPMQQVNT
SSDPLSCFNNGEAPSMWDSHVQNLYGNLGV
> HA-TALE(ng2, C63)-GS-iNES5-cib1-mutNLS
(SEQ ID NO: 167)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
LYPERLRRILTMKHKAKKEENNFSNDSSKVTKELEKTDYIHVRARRGQATDSHSIAERV
RREKISERMKFLQDLVPGCDKITGKAGMLDEIINYVQSLQRQIEFLSMKLAIVNPRPDFD
MDDIFAKEVASTPMTVVPSPEMVLSGYSHEMVHSGYSSEMVNSGYLHVNPMQQVNTS
SDPLSCFNNGEAPSMWDSHVQNLYGNLGV
> HA-TALE(ng2, C63)-NLS-cib1Δ1
(SEQ ID NO: 168)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASGGGGSGGGGSNGAIGGDLLLNFPDMSVLERQR
AHLKYLNPTFDSPLAGFFADSSMITGGEMDSYLSTAGLNLPMMYGETTVEGDSRLSISPE
TTLGTGNFKAAKFDTETKDCNEAAKKMTMNRDDLVEEGEEEKSKITEQNNGSTKSIKK
MKHKAKKEENNFSNDSSKVTLYPERLRRILTKELEKTDYIHVRARRGQATDSHSIAERV
RREKISERMKFLQDLVPGCDKITGKAGMLDEIINYVQSLQRQIEFLSMKLAIVNPRPDFD
MDDIFAKEVASTPMTVVPSPEMVLSGYSHEMVHSGYSSEMVNSGYLHVNPMQQVNTS
SDPLSCFNNGEAPSMWDSHVQNLYGNLGV
> HA-TALE(ng2, C63)-NLS-cib1Δ1
(SEQ ID NO: 169)
MYPYDVPDYASRTRLPSPPAPSPAFSADSFSDLLRQFDPSLFNTSLFDSLPPFG
AHHTEAATGEWDBVQSGLRAADAPPPTMRVAVTAARPPRAKPAPRRRAAQPSDASPA
AQVDLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKR
GGVTAVEAVHAWRNALTGAPLNLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNIGGKQALETVQRLLPVLCQAHGLTPEQVVAIASNGGGKQ
ALETVQRLLPVLCQAHGLTPEQVVAIASNNGGKQALETVQRLLPVLCQAHGLTPHQVV
AIASNIGGKQALETVQRLLPVLCQAHGLTPEQVVAIASNGGGKQALETVQRLLPVLCQA
HGLTPEQVVAIASNNGGKQALETVQRLLPVLCQAHGLTPEQVVAIASNIGGKQALETVQ
RLLPVLCQAHOLTPEQVVAIASNGGGKQALETVQRLLPVLCQAHGLTPEQVVAIASNIG
GKQALETVQRLLPVLCQAHGLTPEQVVAIASNIGGKQALETVQRLLPVLCQAHGLTPEQ
VVAIASNGGGKQALETVQRLLPVLCQAHGLTPEQVVAIASNIGGKQALETVQRLLPVLC
QAHGLTPEQVVAIASHDGGRPALESIVAQLSRPDPALAALTNDHLVALACLGGRPALDA
VKKGLPHAPALIKRTNRRIPERTSHRVAASSPKKKRKVEASNGAIGGDLLLNFPDMSVLE
RQRAHLKYLNPTFDSPLAGFFADSSMITGGEMDSYLSTAGLNLPMMYGETTVEGDSRLS
ISPETTLGTGNFKKRKFDTETKDCNEKKKKMTMNRDDLVEEGEEEKSKITEQNNGSTKS
IKKMKHKAKKEENNFSNDSSKVTKELEKTDYIHVRARRGQATDSHSIAERVRREKISER
MKFLQDLVPGCDKITGKAGMLDEIINYVQSLQRGGSVASTPMTVVPSPEMVLSGYSHE
MVHSGYSSEMVNSGYLHVNPMQQVNTSSDPLSCFNNGEAPSMWDSHVQNLYGNLGV
> HA-TALE(ng2, C63)-NLS-cib1Δ2
(SEQ ID NO: 170)
YPYDVPDYASRTRLPSPPAPSPAFSADSFSDLLRQFDPSLFNTSLFDSLPPFGA
HHTEAATGEWDEVQSGLRAADAPPPTMRVAVTAARPPRAKPAPRRRAAQPSDASPAA
QVDLRTLGYSQQQQEKIKPKVRSTVAQHHEALVGHGFTHAHIVALSQHPAALGTVAVK
YQDMIAALPEATHEAIVGVGKQWSGARALEALLTVAGELRGPPLQLDTGQLLKIAKRG
GVTAVEAVHAWRNALTGAPLNLTPEQVVAIASNGGGKQALETVQRLLPVLCQAHGLTP
EQVVAIASNNGGKQALETVQRLLPVLCQAHGLTPEQVVAIASNIGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASNNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNNGGKQALETVQRLLPVLCQAHGLTPEQVVAIASNIGGKQALETVQRLL
PVLCQAHGLTPEQVVAIASNGGGKQALETVQRLLPVLCQAHGLTPEQVVAIASNIGGKQ
ALETVQRLLPVLCQAHGLTPEQVVAIASNIGGKQALETVQRLLPVLCQAHGLTPEQVVA
IASNGGGKQALETVQRLLPVLCQAHGLTPEQVVAIASNIGGKQALETVQRLLPVLCQAH
QLTPEQVVAIASHDGGRPALESIVAQLSRPDPALAALTNDHLVALACLGGRPALDAVKK
GLPHAPALIKRTNRRIPERTSHRVAASSPKKKRKVEASNGAIGGDLLLNFPDMSVLERQR
AHLKYLNPTFDSPLAGFFADSSMITGGEMDSYLSTAGLNLPMMYGETTVEGDSRLSISPE
TTLGTGNFKKRKFDTETKDCNEKKKKMTMNRDDLVEEGEEEKSKITEQNNGSTKSIKK
MKHKAKKEENNFSNDSSKVTKELEKTDYIHVRARRGQATDSHSIAERVRREKISERMKF
LQDLVPGCDKITGKAGMLDEIINYVQSLQRQIEFLSMKLAIVNPRPDFDMDDIFAKEVAS
TPMTVVPSPEMVLSGYGGSPLSCFNNGEAPSMWDSHVQNLYGNLGV
> alpha-importin-NLS-CRY2PHR-NLS-VP64_2A_GFP
(SEQ ID NO: 171)
MKRPAATKKAGQAKKKKKMDKKTIVWFRRDLRIEDNPALAAAAHEGSVFP
VFIWCPEEEGQFYPGRASRWWMKQSLAHLSQSLKALGSDLTLIKTHNTISAILDCIRVTG
ATKVVFNHLYDPVSLVRDHTVKEKLVERGISVQSYNGDLLYEPWEIYCEKGKPFTSFNS
YWKKCLDMSIESVMLPPPWRLMPITAAAEAIWACSIEELGLENEAEKPSNALLTRAWSP
GWSNADKLLNEFIEKQLIDYAKNSKKVVGNSTSLLSPYLHFGEISVRHVFQCARMKQII
WARDKNSEGEESADLFLRGIGLREYSRYICFNFPFTHEQSLLSHLRFFPWDADVDKFKA
WRQGRTGYPLVDAGMRELWATGWMHNRIRVIVSSFAVKFLLLPWKWGMKYFWDTLL
DADLECDILGWQYISGSIPDGHELDRLDNPALQGAKYDPEGEYIRQWLPELARLPTEWIH
HPWDAPLTVLKASGVELGTNYAKPIVDIDTARELLAKAISRTREAQIMIGAAPASPKKKR
KVEASGSGRADALDDFDLDMLGSDALDDFDLDMLGSDALDDFDLDMLGSDALDDFDL
DMLINSRGSGEGRGSLLTCGDVEENPGPVSKGEELFTGVVPILVELDGDVNGHKFSVSG
EGEGDATYGKLTLKFICTTGKLPVPWPTLVTTLTYGVQCFSRYPDHMKQHDFFKSAMP
EGYVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQSAL
SKDPNEKRDHMVLLEFVTAAGITLGMDELYKV
> mutNES-CRY2PHR-NLS-VP64_2A_GFP
(SEQ ID NO: 172)
MEQKLISEEDLKMDKKTIVWFRRDLRIEDNPALAAAAHEGSVFPVFIWCPEEE
GQFYPGRASRWWMKQSLAHLSQSLKAAGSDATLIKTHNTISAILDCIRVTGATKVVFNH
LYDPVSLVRDHTVKEKLVERGISVQSYNGDLLYEPWEIYCEKGKPFTSFNSYWKKCLD
MSIESVMLPPPWRLMPITAAAEAIWACSIEELGLENEAEKPSNALLTRAWSPGWSNADK
LLNEFIEKQLIDYAKNSKKVVGNSTSLLSPYLHFGEISVRHVFQCARMKQIIWARDKNSE
GEESADLFLRGIGLREYSRYICFNFPFTHEQSLLSHLRFFPWDADVDKFKAWRQGRTGYP
LVDAGMRELWATGWMHNRIRVIVSSFAVKFLLLPWKWGMKYFWDTLLDADLECDILG
WQYISGSIPDGHELDRLDNPALQGAKYDPEGEYIRQWLPELARLPTEWIHHPWDAPLTV
LKASGVELGTNYAKPIVDIDTARELLAKAISRTREAQIMIGAAPASPKKKRKVEASGSGR
ADALDDFDLDMLGSDALDDFDLDMLGSDALDDFDLDMLGSDALDDFDLDMLINSRGS
GEGRGSLLTCGDVEENPGPVSKGEELFTGVVPILVELDGDVNGHKFSVSGEGEGDATYG
KLTLKFTCTTGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSALSKDPNEKRD
HMVLLEFVTAAGITLGMDELYKV
> CRY2PHR-NLS-VP64-NLS_2A_GFP
(SEQ ID NO: 173)
MKMDKKTIVWFRRDLRIEDNPALAAAAHEGSVFPVFIWCPEEEGQFYPGRAS
RWWMKQSLAHLSQSLKALGSDLTLIKTHNTISAILDCIRVTGATKVVFNHLYDPVSLVR
DHTVKEKLVERGISVQSYNGDLLYEPWEIYCEKGKPFTSFNSYWKKCLDMSIESVMLPP
PWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLR
GIGLREYSRYICFNFPFTHEQSLLSHLRFFPWDADVDKFKAWRQGRTGYPLVDAGMREL
WATGWMHNRIRVIVSSFAVKFLLLPWKWGMKYFWDTLLDADLECDILGWQYISGSIPD
GHELDRLDNPALQGAKYDPEGEYIRQWLPELARLPTEWIHHPWDAPLTVLKASGVELG
TNYAKPIVDIDTARELLAKAISRTREAQIMIGAAPASPKKKRKVEASGSGRADALDDFDL
DMLGSDALDDFDLDMLGSDALDDFDLDMLGSDALDDFDLDMLINSPKKKRKVEASSR
GSGEGRGSLLTCGDVEENPGPVSKGEELFTGVVPILVELDGDVNGHKFSVSGEGEGDAT
YGKLTLKFICTTGKLPVPWPTLVTTLTYGVQCFSRYPDHMKQHDFFKSAMPEGYVQER
TIFFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPNEK
RDHMVLLEFVTAAGITLGMDELYKV
> Neurog2-TALE(N240,C63)-PYL
(SEQ ID NO: 174)
MSRTRLPSPPAPSPAFSADSFSDLLRQFDPSLFNTSLFDSLPPFGAHHTEAATG
EWDEVQSGLRAADAPPPTMRVAVTAARPPRAKPAPRRRAAQPSDASPAAQVDLRTLGY
SQQQQEKIKPKVRSTVAQHHEALVGHGFTHAHIVALSQHPAALGTVAVKYQDMIAALP
EATHEAIVGVGKQWSGARALEALLTVAGELRGPPLQLDTGQLLKIAKRGGVTAVEAVH
AWRNALTGAPLNLTPEQVVAIASHNGGKQALETVQRLLPVLCQAHGLTPEQVVAIASNI
GGKQALETVQRLLPVLCQAHGLTPEQVVAIASNIGGKQALETVQRLLPVLCQAHGLTPE
QVVAIASNGGGKQALETVQRLLPVLCQAHGLTPEQVVAIASHNGGKQALETVQRLLPV
LCQAHGLTPEQVVAIASNIGGKQALETVQRLLPVLCQAHGLTPEQVVAIASNGGGKQAL
ETVQRLLPVLCQAHGLTPEQVVAIASHNGGKQALETVQRLLPVLCQAHGLTPEQVVAIA
SNIGGKQALETVQRLLPVLCQAHGLTPEQVVAIASNGGGKQALETVQRLLPVLCQAHGL
TPEQVVAIASNIGGKQALETVQRLLPVLCQAHGLTPEQVVAIASNIGGKQALETVQRLLP
VLCQAHGLTPEQVVAIASNGGGRPALESIVAQLSRPDPALAALTNDHLVALACLGGRPA
LDAVKKGLPHAPALiKRTNRRIPERTSIIRVAASMANSESSSSPVNEEENSQRISTLHHQT
MPSDLTQDEFTQLSQSIAEFHTYQLGNGRCSSLLAQRIHAPPETVWSVVRRFDRPQIYKH
FIKSCNVSEDFEMRVGCTRDVNVISGLPANTSRERLDLLDDDRRVTGFSITGGEHRLRNY
KSVTTVHRFEKEEEEERIWTVVLESYVVDVPEGNSEEDTRLFADTVIRLNLQKLASITEA
MNRNNNNNNSSQVR
>ABI-NLS-VP64
(SEQ ID NO: 175)
MVPLYGFTSICGRRPEMEAAVSTIPRFLQSSSGSMLDGRFDPQSAAHFFGVYD
GHGGSQVANYCRERMHLALAEEIAKEKPMLCDGDTWLEKWKKALFNSFLRVDSEIESV
APETVGSTSVVAVVFPSHIFVANCGDSRAVLCRGKTALPLSVDHKPDREDEAARIEAAG
GKVIQWNGARVFGVLAMSRSIGDRYLKPSIIPDPEVTAVKRVKEDDCLILASDGVWDV
MTDEEACEMARKRILLWHKKNAVAGDASLLADERRKEGKDPAAMSAAEYLSKLAIQR
GSKDNISVVVVDLKPRRKLKSKPLNASPKKKRKVEASGSGRADALDDFDLDMLGSDAL
DDFDLDMLGSDALDDFDLDMLGSDALDDFDLDMLIN
>hSpCas9(D10A,H840A)-Linker-NLS-VF64
(SEQ ID NO: 176)
MDKKYSIGLAIGTNSVGWAVTIDEYKVPSKKFKVLGNTDRHSIKNLIGALLF
DSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFIVYNELTKVKYVTE
GMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDAIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGDSAGGGGSGGGGSGGGGSGPKKKRKVAAAGSGRADALDDFDLDMLGSDALDDF
DLDMLGSDALDDFDLDMLGSDALDDFDLDMLIN

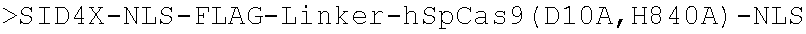

(SEQ ID NO: 177)
MGSGMNIQMLLEAADYLERREREAEHGYASMLPGSGMNIQMLLEAADYLE
RREREAEHGYASMLPGSGMNIQMLLEAADYLERREREAEHGYASMLPGSGMNIQMLL

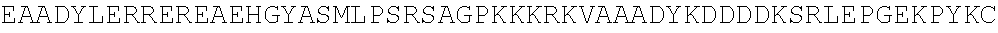

PECGKSFSQSGALTRHQRTHTRDKKYSIGLAIGTNSVGWAVITDEYKVPSKKFKVLGNT
DRHSIKKNLIGALLFDSGETAEATRLKRTARRRYTRRKNRICYLQEIFSNEMAKVDDSFF
HRLEESPLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDNL
LAQIGDQYADLFLAAKNLSDAILLSDIURVNTEITKAPLSASMIKRYDEHHQDLTLLKAL
VRQQLPEKYKEIFFDQSKNGYAGYEDGGASQEEFYKFIKPILEKMDGTEELLVKLNREDL
LRKQRTFDNGSIPHQIHLGELHAILRRQEDFYPFIKDNRBKIEKILTFRIPYYVGPLARGNS
RFAWMTRKSEETITPWNFEEVVDKGASAQSFIERMTNFDKNLPNEKVLPKHSLLYEYFT
VYNELTKVKYVTEGMRKPAFLSGEQKKAIVDLLEICTNRKVTVKQLKEDYFKKIECFDS
VEISGVEDRFKASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQL
IHDDSLTFKEDIQKAQVSGQGDSLHEIHANLAGSPAIKKGILQTVKVVDELVKVMGRHK
PENIVIEMARENQTTQKGQKNSRERMKRIEEGIKELGSQILKEHPVENTQLQNEKLYLYY
LQNGRDMYVDQELDINRLSDYDVDAIVFQSFLKDDSIDNKVLTRSDKNRQKSDNVFSEE
VVKKMKNYWRQLLNAKLITQRKFDNLTKAERGGLSHLDKAGFIKRQLVETRQITKHVA
QILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGELQKGNELALP
SKYVNFLYLASHYEKLKGSPEDNEQKQLFVEQHKGYLDEIIEQISEFSKRVILADANLDK
VLSAYNKHRDKPIREQAENIIHLFTLTNLGAPAAPKYFDTTIDRKRYTSTKEVLDATLIHQ
SITGLYETRIDLSQLGGDKRPAATKKAGQAKKKK
Epigenetic effector domain sequences
>hs_NCoR
(SEQ ID NO: 178)
ASSPKKKRKVEASMNGLMEDPMKVYKDRQFMNVWTDHEKEIFKDKFIQHP
KNFGLIASYLERKSVPDCVLYYYLTKKNENYKEF
>pf_Sir2A
(SEQ ID NO: 179)
ASSPKKKRKVEASMGNLMISFLKKDTQSITLEELAKIIKKCKHVVALTGSGTS
AESNIPSFRGSSNSIWSKYDPRIYGTIWGFWKYPEKIWEVIRDISSDYEIEINNGHVALSTL
ESLGYLKSVVTQNVDGLHEASGNTKVISLHGNVFEAVCCTCNKIVKLNKIMLQKTSHFM
HQLPPECPCGGIFKPNIILFGEVVSSDLLKEAEEEIAKCDLLLVIGTSSTVSTATNLCHFAC
KKKKKIVEINISKTYITNKMSDYHVCAKFSELTKVANILKGSSEKNKKIMEF
>nc_DIM5
(SEQ ID NO: 180)
ASSPKKKRKVEASMEKAFRPHFFNHGKPDANPKEKKNCHWCQIRSFATHAQ
LPISIVNREDDAFLNPNFRFIDHSIIGKNVPVADQSFRVGCSCASDEECMYSTCQCLDEMA
PDSDEEADPYTRKKRFAYYSQGAKKGLLRDRVLQSQEPIYECHQGCACSKDCPNRVVE
RGRTVPLQIFRTKDRGWGVKCPVNIKRGQFVDRYLGEIITSEEADRRRAESTIARRKDVY
LFALDKFSDPDSLDPLLAGQPLEVDGEYMSGPTRFINHSCDPNMAIFARVGDHADKHIH
DLALFAIKDIPKGTELTFDYVNGLTGLESDAHDPSKISEMTKCLCGTAKCRGYLWEF
>sc_HST2
(SEQ ID NO: 181)
ASSPKKKRKVEASTEMSVRKIAAHMKSNPNAKVIFMVGAGISTSCGIPDFRSP
GTGLYHNLARLKLPYPEAVFDVDFFQSDPLPFYTLAKELYPGNFRPSKFHYLLKLFQDK
DVLKRVYTQNIDTLERQAGVKDDLIIEAHGSFAHCHCIGCGKVYPPQVFKSKLAEHPIKD
FVKCDVCGELVKPAIVFFGEDLPDSFSETWLNDSEWLREKITTSGKHPQQPLVIVVGTSL
AVYPFASLPEEIPRKVKRVLCNLETVGDFKANKRPTDLIVHQYSDEFAEQLVEELGWQE
DFEKILTAQGGMGEF
>hs_SIRT3
(SEQ ID NO: 182)
ASSPKKKRKVEASMVGAGISTPSGIPDFRSPGSGLYSNLQQYDLPYPEAIFELP
FFFHNPKPFFTLAKELYPGNYKPNVTHYFLRLLHDKGLLLRLYTQNIDGLERVSGIPASK
LVEAHGTFASATCTVCQRPFPGEDIRADVMADRVPRCPVCTGVVKPDIVFFGEPLPQRFL
LHVVDFPMADLLLILGTSLEVEPFASLTEAVRSSVPRLLINRDLVGPLAWHPRSRDVAQL
GDVVHGVESLVELLGWTEEMRDLVQRETGKLDGPDKEF
>hs_NIPP1
(SEQ ID NO: 183)
ASSPKKKRKVEASMAAAANSGSSLPLFDCPTWAGKPPPGLHLDVVKGDKLIE
KLIIDEKKYYLFGRNPDLCDFTIDHQSCSRVHAALVYHKHLKRVFLIDLNSTHGTFLGHI
RLEPHKPQQIPIDSTVSFGASTRAYTLREKPQTLPSAVKGDEKMGGEDDELKGLLGLPEE
ETELDNLTEFNTAHNKRISTLTIEEGNLDIQRPKRKRKNSRVTFSEDDEIINPEDVDPSVGR
FRNMVQTAVVPVKKKRVEGPGSLGLEESGSRRMQNFAFSGGLYGGLPPTHSEAGSQPH
GIHGTALIGGLPMPYPNLAPDVDLTPVVPSAVNMNPAPNPAVYNPEAVNEEF
>ct_NUE
(SEQ ID NO: 184)
ASSPKKKRKVEASMTTNSTQDTLYLSLHGGIDSAIPYPVRRVEQLLQFSFLPE
LQFQNAAVKQRIQRLCYREEKRLAVSSLAKWLGQLHKQRLRAPKNPPVAICWINSYVG
YGVFARESIPAWSYIGEYTGILRRRQALWLDENDYCFRYPVPRYSFRYFTIDSGMQGNV
TRFINHSDNPNLEAIGAFENGIFHIIIRAIKDILPGEELCYHYGPLYWKHRKKREEFVPQEE
EF
>hs_MBD2b
(SEQ ID NO: 185)
ASSPKKKRKVEASARYLGNTVDLSSFDFRTGKMMPSKLQKNKQRLRNDPLN
QNKGKPDLNTTLPIRQTASIFKQPVTKVTNHPSNKVKSDPQRMNEQPRQLFWEKRLQGL
SASDVTEQIIKTMELPKGLQGVGPGSNDETLLSAVASALHTSSAPITGQVSAAVEKNPAV
WLNTSQPLCKAFIVTDEDIRKQEERVQQVRKILEDALMADILSRAADTEEMDIEMDSGD
EAEF
>ca_HST2
(SEQ ID NO: 186)
ASSPKKKRKVEASMPSLDDILKPVAEAVKNGKKVTFFNGAGISTGAGIPDFRS
PDTGLYANLAKLNLPFAEAVFDIDFFKEDPKPFYTLAEELYPGNFAPTKFHHFIKLLQDQ
GSLKRVYTQNIDTLERLAGVEDKYIVEAHGSFASNHCVDCHKEMTTETLKTYMKDKKI
PSCQHCEGYVKPDIVFFGEGLPVKFFDLWEDDCEDVEVAIVAGTSLTVFPFASLPGEVNK
KCLRVLVNKEKVGTFKHEPRKSDIIALHDCDIVAERLCTLLGLDDKLNEVYEKEKIKYSK
AETKEIKMHEIEDKLKEEAHLKEDKHTTKVDKKEKQNDANDKELEQLIDKAKAEF
>hs_PHF19
(SEQ ID NO: 187)
ASSPKKKRKVEASMENRALDPGTRDSYGATSHLPNKGALAKVKNNFKDLMS
KLTEGQYVLCRWTDGLYYLGKIKRVSSSKQSCLVTFEDNSKYWVLWKDIQHAGVPGEE
PKCNICLGKTSGPLNEILICGKCGLGYHQQCHIPIAGSADQPLLTPWFCRRCIFALAVRKG
GALKKGAIARTLQAVKMVLSYQPEELEWDSPHRTNQQQCYCYCGGPGEWYLRMLQCY
RCRQWFHEACTQCLNEPMMFGDRFYLFFCSVCNQGPGGSGSDSSAEGASVPERPDEGID
SHTFESISEDDSSLSHLKSSITNYFGAAGRLACGEKYQVLARRVTPEGKVQYLVEWEGTT
PYEF
>hs_HDAC11
(SEQ ID NO: 188)
ASSPKKKRKVEASMLHTTQLYQHVPETRWPIVYSPRYNITFMGLEKLHPFDA
GKWGKVINFLKEEKLLSDSMLVEAREASEEDLLVVHTRRYLNELKWSFAVATFTEIPPVI
FLPNFLVQRKVLRPLRTQTGGTIMAGKLAVERGWAINVGGGFHHCSSDRGGGFCAYAD
ITLAIKFLFERVEGISRATIIDLDAHQGNGHERDFMDDKRVYIMDVYNRHIYPGDRFAKQ
AIRRKVELEWGTEDDEYLDKVERNIKKSLQEHLPDVVVYNAGTDILEGDRLGGLSISPA
GIVKRDELVFRMVRGRRVPILMVTSGGYQKRTARIIADSILNLFGLGLIGPESPSVSAQNS
DTPLLPPAVPEF
>m1_MesoLo4
(SEQ ID NO: 189)
ASSPKKKRKVEASMPLQIVHHPDYDAGFATNHRFPMSKYPLLMEALRARGL
ASPDALNTTEPAPASWLKLAHAADYVDQVISCSVPEKIEREIGFPVGPRVSLRAQLATGG
TILAARLALRHGIACNTAGGSHHARRAQGAGFCTFNDVAVASLVLLDEGAAQNILVVD
LDVHQGDGTADILSDEPGVFTFSMHGERNYPVRKIASDLDIALPDGTGDAAYLRRLATIL
PELSARARWDIVFYNAGVDVHAEDRLGRLALSNGGLRARDEMVIGHFRALGIPVCGVI
GGGYSTDVPALASRHAILFEVASTYAEF
>pbcv1_vSET
(SEQ ID NO: 190)
ASSPKKKRKVEASMFNDRVIVKKSPLGGYGVFARKSFEKGELVEECLCIVRH
NDDWGTALEDYLFSRKNMSAMALGFGAIFNHSKDPNARHELTAGLKRMRIFTIKPIAIG
EEITISYGDDYWLSRPRLTQNEF
>at_KYP
(SEQ ID NO: 191)
ASSPKKKRKVEASDISGGLEFKGIPATNRVDDSPVSPTSGFTYIKSLIIEPNVIIP
KSSTGCNCRGSCTDSKKCACAKLNGGNFPYVDLNDGRLIESRDVVFECGPHCGCGPKC
VNRTSQKRLRFNLEVFRSAKKGWAVRSWEYIPAGSPVCEYIGVVRRTADVDTISDNEYI
FEIDCQQTMQGLGGRQRRLRDVAVPMNNGVSQSSEDENAPEFCIDAGSTGNFARFINHS
CEPNLFVQCVLSSHQDIRLARVVLFAADNISPMQELTYDYGYALDSVHEF
>tg_TgSET8
(SEQ ID NO: 192)
ASSPKKKRKVEASASRRTGEFLRDAQAPSRWLKRSKTGQDDGAFCLETWLA
GAGDDAAGGERGRDREGAADKAKQREERRQKELEERFEEMKVEFEEKAQRMIARRAA
LTGEIYSDGKGSKKPRVPSLPENDDDALIEIIIDPEQGILKWPLSVMSIRQRTVIYQECLRR
DLTACIHLTKVPGKGRAVFAADTILKDDFVVEYKGELCSEREAREREQRYNRSKVPMGS
FMFYFKNGSRMMAIDATDEKQDFGPARLINHSRRNPNMTPRAITLGDFNSEPRLIFVARR
NIEKGEELLVDYGERDPDVIKEHPWLNSEF
>hs_SIRT6
(SEQ ID NO: 193)
ASSPKKKRKVEASMSVNYAAGLSPYADKGKCGLPEIFDPPEELERKVWELAR
LVWQSSSVVFHTGAGISTASGIPDFRGPHGVWTMEERGLAPKFDTTFESARPTQTHMAL
VQLERVGLLRFLVSQNVDGLHVRSGFPRDKLAELHGNMFVEECAKCKTQYVRDTVVG
TMGLKATGRLCTVAKARGLRACRGELRDTILDWEDSLPDRDLALADEASRNADLSITL
GTSLQIRPSGNLPLATKRRGGRLVIVNLQPTKHDRHADLRIHGYVDEVMTRLMKHLGLE
IPAWDGPRVLERALPPLEF
>ce_Set1
(SEQ ID NO: 194)
ASSPKKKRKVEASMKVAAKKLATSRMRKDRAAAASPSSDIENSENPSSLASH
SSSSGRMTPSKNTRSRKGVSVKDVSNHKITEFFQVRRSNRKTSKQISDEAKHALRDTVL
KGTNERLLEVYKDVVKGRGIRTKVNFEKGDFVVEYRGVMMEYSEAKVIEEQYSNDEEI
GSYMYFFEHNNKKWCIDATKESPWKGRLINHSVLRPNLKTKVVEIDGSHHLILVARRQI
AQGEELLYDYGDRSAETIAKNPWLVNTEF
>mm_G9a
(SEQ ID NO: 195)
ASSPKKKRKVEASVRTEKIICRDVARGYENVPIPCVNGVDGEPCPEDYKYISE
NCETSTMNIDRNITHLQHCTCVDDCSSSNCLCGQLSIRCWYDKDGRLLQEFNKIEPPLIFE
CNQACSCWRSCKNRVVQSGIKVRLQLYRTAKMGWGVRALQTIPQGTFICEYVGELISD
AEADVREDDSYLFDLDNKDGEVYCIDARYYGNISRFINHLCDPNIIPVRVFMLHQDLRFP
RIAFFSSRDIRTGEELGFDYGDRFWDIKSKYFTCQCGSEKCKHSAEAIALEQSRLARLDP
HPELLPDLSSLPPINTEF
>hs_SIRT5
(SEQ ID NO: 196)
ASSPKKKRKVEASSSSMADFRKFFAKAKHIVIISGAGVSAESGVPTFRGAGGY
WRKWQAQDLATPLAFAHNPSRVWEFYHYRREVMGSKEPNAGHRAIAECETRLGKQGR
RVVVITQNIDELHRKAGTKNLLEIHGSLFKTRCTSCGVVAENYKSPICPALSGKGAPEPG
TQDASIPVEKLPRCEEAGCGGLLRPHVVWFGENLDPAILEEVDRELAHCDLCLVVGTSS
VVYPAAMFAPQVAARGVPVAEFNTETTPATNRFRFHFQGPCGTTLPEALACHENETVSE
F
>x1_HDAC8
(SEQ ID NO: 197)
ASSPKKKRKVEASMSRVVKPKVASMEEMAAFHTDAYLQHLHKVSEEGDND
DPETLEYGLGYDCPITEGIYDYAAAVGGATLTAAEQLIEGKTRIAVNWPGGWHHAKKD
EASGFCYLNDAVLGILKLREKFDRVLYVDMDLHHGDGVEDAFSFTSKVMTVSLHKFSP
GFFPGTGDVSDIGLGKGRYYSINVPLQDGIQDDKYYQICEGVLKEVFTTFNPEAVVLQLG
ADTIAGDPMCSFNMTPEGIGKCLKYVLQWQLPTLILGGGGYHLPNTARCWTYLTALIVG
RTLSSEIPDHEFFTEYGPDYVLEITPSCRPDRNDTQKVQEILQSIKGNLKRVVEF
>mm_HP1a
(SEQ ID NO: 198)
ASSPKKKRKVEASMKEGENNKPREKSEGNKRKSSFSNSADDIKSKKKREQSN
DIARGFERGLEPEKIIGATDSCGDLMFLMKWKDTDEADLVLAKEANVKCPQIVIAFYEE
RLTWHAYPEDAENKEKESAKSEF
>at_HDT1
(SEQ ID NO: 199)
ASSPKKKRKVEASMEFWGIEVKSGKPVTVTPEEGILIHVSQASLGECKNKKG
EFVPLHVKVGNQNLVLGTLSTENIPQLFCDLVFDKEFELSHTWGKGSVYFVGYKTPNIEP
QGYSEEEEEEEEEVPAGNAAKAVAKPKAKPAEVKPAVDDEEDESDSDGMDEDDSDGE
DSEEEEPTPKKPASSKKRANETTPKAPVSAKKAKVAVTPQKTDEKKKGGKAANQSEF
>mm_SAll
(SEQ ID NO: 200)
ASSPKKKRKVEASMSRRKQAKPQHFQSDPEVASLPRRDGDTEKGQPSRPTKS
KDAHVCGRCCAEFFELSDLLLHKKSCTKNQLVLIVNESPASPAKTFPPGPSLNDEF
>hs_SETD8
(SEQ ID NO: 201)
ASSPKKKRKVEASSCDSTNAAIAKQALKKPIKGKQAPRKKAQGKTQQNRKL
TDFYPVRRSSRKSKAELQSEERKRIDELIESGKEEGMKIDLIDGKGRGVIATKQFSRGDFV
VEYHGDLIEITDAKKREALYAQDPSTGCYMYYFQYLSKTYCVDATRETNRLGRLINHSK
CGNCQTKLHDIDGVPHLILIASRDIAAGEELLYDYGDRSKASIEAFPWLKHEF
>sc_RPD3
(SEQ ID NO: 202)
ASSPKKKRKVEASRRVAYFYDADVGNYAYGAGHPMKPHRIRMAHSLIMNY
GLYKKMEIYRAKPATKQEMCQFHTDEYIDFLSRVTPDNLEMFKRESVKFNVGDDCPVF
DGLYEYCSISGGGSMEGAARLNRGKCDVAVNYAGGLHHAKKSEASGFCYLNDIVLGIIE
LLRYHPRVLYIDIDVHHGDGVEEAFYTTDRVMTCSFHKYGEFFPGTGELRDIGVGAGKN
YAVNVPLRDGIDDATYRSVFEPVIKKIMEWYQPSAVVLQCGGDSLSGDRLGCFNLSME
GHANCVNYVKSFGIPMMVVGGGGYTMRNVARTWCFETGLLNNVVLDKDLPYEF
>cc_CobB
(SEQ ID NO: 203)
ASSPKKKRKVEASMEKPRVLVLTGAGISAESGIRTFRAADGLWEEHRVEDVA
TPEGFDRDPELVQAFYNARRRQLQQPEIQPNAAHLALAKLQDALGDRFLLVTQNIDNLH
ERAGNTNVIHMHGELLKVRCSQSGQVLDWTGDVTPEDKCHCCQFPAPLRPHVVWFGE
MPLGMDEIYMALSMADIFIAIGTSGHVYPAAGFVHEAKLHGAHTVELNLEPSQVGNEFA
EKYYGPASQVVPEFVEKLLKGLKAGSIAEF (SEQ ID NO: 203)
>hs_SUV39HI
(SEQ ID NO: 204)
ASSPKKKRKVEASNLKCVRILKQFHKDLERELLRRHHRSKTPRHLDPSLANY
LVQKAKQRRALRRWEQELNAKRSHLGRITVENEVDLDGPPRAFVYINEYRVGEGITLNQ
VAVGCECQDCLWAPTGGCCPGASLHKFAYNDQGQVRLRAGLPIYECNSRCRCGYDCP
NRVVQKGIRYDLCIFRTDDGRGWGVRTLEKIRKNSFVMEYVGEIITSEEAERRGQIYDRQ
GATYLFDLDYVEDVYTVDAAYYGNISHFVNHSCDPNLQVYNVFIDNLDERLPRIAFFAT
RTIRAGEELTFDYNMQVDPVDMESTRMDSNFGLAGLPGSPKKRVRIECKCGTESCRKYL
FEF
>hs_RCORl
(SEQ ID NO: 205)
ASSPKKKRKVEASSNSWEEGSSGSSSDEEHGGGGMRVGPQYQAVVPDFDPA
KLARRSQERDNLGMLVWSPNQNLSEAKLDEYIAIAKEKHGYNMEQALGMLFWHKHNI
EKSLADLPNFTPFPDEWTVEDKVLFEQAFSFHGKTFHRIQQMLPDKSIASLVKFYYSWK
KTRTKTSVMDRHARKQKREREESEDELEEANGNNPIDIEVDQNKESKKEVPPTETVPQV
KKEKHSTEF
>hs_sin3a
(SEQ ID NO: 206)
ASSPKKKRKVEASYKESVHLETYPKERATEGIAMEIDYASCKRLGSSYRALP
KSYQQPKCTGRTPLCKEVLNDTWVSFPSWSEDSTFVSSKKTQYEEHIYRCEDERFELDV
VLETNLATIRVLEAIQKKLSRLSAEEQAKFRLDNTLGGTSEVIHRKALQRIYADKAADIID
GLRKNPSIAVPIVLKRLKMKEEEWREAQRGFNKVWREQNEKYYLKSLDHQGINFKQN
D
TKVLRSKSLLNEIESIYDERQEQATEENAGVPVGPHLSLAYEDKQILEDAAALIIHHVKR
QTGIQKEDKYKIKQIMHHFIPDLLFAQRGDLSDVEEEEEEEMDVDEATGAVEF
>at_SUVR4
(SEQ ID NO: 207)
ASSPKKKRKVEASQSAYLHVSLARISDEDCCANCKGNCLSADFPCTCARETS
GEYAYTKEGLLKEKFLDTCLKMKKEPDSFPKVYCKDCPLERDHDKGTYGKCDGHLIRK
FIKECWRKCGCDMQCGNRVVQRGIRCQLQVYFTQEGKGWGLRTLQDLPKGTFICEYIG
EILTNTELYDRNVRSSSERHTYPVTLDADWGSEKDLKDEEALCLDATICGNVARFINHR
CEDANMIDIPIEIETPDRHYYHIAFFTLRDVKAMDELTWDYMIDFNDKSHPVKAFRCCC
GSESCRDRKIKGSQGKSIERRKIVS AKKQQGSKEVSKKRKEF
>rn_McCP2_NLS
(SEQ ID NO: 208)
ASSPKKKRKVEASVQVKRVLEKSPGKLLVKMPFQASPGGKGEGGGATTSAQ
VMVIKRPGRKRKAEADPQAIPKKRGRKPGSVVAAAAAEAKKKAVKESSIRSVQETVLPI
KKRKTRETVSIEVKEVVKPLLVSTLGEKSGKGLKTCKSPGRKSKESSPKGRSSSASSPPK
KEHHHHHHHAESPKAPMPLLPPPPPPEPQSSEDPISPPEPQDLSSSICKEEKMPRAGSLESD
GCPKEPAKTQPMVAAAATTTTTTTTTVAEKYKHRGEGERKDIVSSSMPRPNREEPVDSR
TPVTERVSEF
>mm_SET-TAFl B
(SEQ ID NO: 209)
ASSPKKKRKVEASMAPKRQSAILPQPKKPRPAAAPKLEDKSASPGLPKGEKE
QQEAIEHIDEVQNEIDRLNEQASEEILKVEQKYNKLRQPFFQKRSELIAKIPNFWVTTFVN
HPQVSALLGEEDEEALHYLTRVEVTEFEDIKSGYRIDFYFDENPYFENKVLSKEFHLNES
GDPSSKSTEIKWKSGKDLTKRSSQTQNKASRKRQHEEPESFFTWFTDHSDAGADELGEV
IKDDIWPNPLQYYLVPDMDDEEGEAEDDDDDDEEEEGLEDIDEEGDEDEGEEDDDEDE
GEEGEEDEGEDDEF
>ce_Set4
(SEQ ID NO: 210)
ASSPKKKRKVEASMQLHEQIANISVTFNDIPRSDHSMTPTELCYFDDFATTLV
VDSVLNFTTHKMSKKRRYLYQDEYRTARTVMKTFREQRDWTNAIYGLLTLRSVSHFLS
KLPPNKLFEFRDHIVRFLNMFILDSGYTIQECKRYSQEGHQGAKLVSTGVWSRGDKIERL
SGVVCLLSSEDEDSILAQEGSDFSVMYSTRKRCSTLWLGPGAYINHDCRPTCEFVSHGST
AHIRVLRDMVPGDEITCFYGSEFFGPNNIDCECCTCEKNMNGAFSYLRGNENAEPIISEK
KTKYELRSRSEF

Photostimulation Hardware Control Scripts

The following Arduino script was used to enable the individual control of each 4-well column of a light-stimulated 24-well plate

```
//Basic control code for LITE LED array using Arduino UNO
//LED column address initialization to PWM-ready Arduino outputs
int led1_pin = 3;
int led2_pin = 5;
int led3_pin = 6;
int led4_pin = 9;
int led5_pin = 10;
int led6_pin = 11;
//Maximum setting for Arduino PWM
int uniform_brightness = 255;
//PWM settings for individual LED columns
int led1_brightness = uniform_brightness/2;
int led2_brightness = uniform_brightness/2;
int led3_brightness = uniform_brightness/2;
int led4_brightness = uniform_brightness/2;
int led5_brightness = uniform_brightness/2;
int led6_brightness = uniform_brightness/2;
//' on' time in msec
unsigned long uniform_stim_time = 1000; /
//individual ' on' time settings for LED columns
unsigned long led1_stim_time = uniform_stim_time;
unsigned long led2_stim_time = uniform_stim_time;
unsigned long led3_stim_time = uniform_stim_time;
unsigned long led4_stim_time = uniform_stim_time;
unsigned long led5_stim_time = uniform_stim_time;
unsigned long led6_stim_time = uniform_stim_time;
//' off' time in msec
unsigned long uniform_off_time = 14000;
//individual 'off' time settings for LED columns
unsigned long led1_off_time = uniform_off_time;
unsigned long led2_off_time = uniform_off_time;
unsigned long led3_off_time = uniform_off_time;
unsigned long led4_off_time = uniform_off_time;
unsigned long led5_off_time = uniform_off_time;
unsigned long led6_off_time = uniform_off_time;
unsigned long currentMillis = 0;
//initialize timing and state variables
unsigned long led1_last_change = 0;
unsigned long led2_last_change = 0;
unsigned long led3_last_change = 0;
unsigned long led4_last_change = 0;
unsigned long led5_last_change = 0;
unsigned long led6_last_change = 0;
int led1_state = HIGH;
int led2_state = HIGH;
int led3_state = HIGH;
int led4_state = HIGH;
int led5_state = HIGH;
int led6_state = HIGH;
unsigned long led1_timer = 0;
unsigned long led2_timer = 0;
unsigned long led3_timer = 0;
unsigned long led4_timer = 0;
unsigned long led5_timer = 0;
unsigned long led6_timer = 0;
void setup( ) {
  // setup PWM pins for output
  pinMode (led1_pin, OUTPUT);
  pinMode (led2_pin, OUTPUT);
  pinMode (led3_pin, OUTPUT);
  pinMode (led4_pin, OUTPUT);
  pinMode (led5_pin, OUTPUT);
  pinMode (led6_pin, OUTPUT);
  //LED starting state
  analogWrite (led1_pin, led1_brightness);
  analogWrite (led2_pin, led2_brightness);
  analogWrite (led3_pin, led3_brightness);
  analogWrite (led4_pin, led4_brightness);
  analogWrite (led5_pin, led5_brightness);
  analogWrite (led6_pin, led6_brightness);
}
void loop( ) {
  currentMillis = millis ( );
  //identical timing loops for the 6 PWM output pins
  led1_timer = currentMillis - led1_last_change;
  if (led1_state == HIGH) { //led state is on
    if (led1_timer >= led1_stim_time){ //TRUE if stim time is complete
      analogWrite (led1_pin, 0); //turn LED off
      led1_state = LOW;  //change LED state variable
      led1last_change = currentMillis; //mark time of most recent change
    }
  }
  else{ //led1 state is off
    if (led1_timer >= led1_off_time) { //TRUE if off time is complete
      analogWrite (led1_pin, led1_brightness); //turn LED on
      led1_state = HIGH; //change LED state variable
      led1_last_change = currentMillis; //mark time of most recent change
    }
  }
  led2_timer = currentMillis - led2_last_change;
  if (led2_state == HIGH){
    if (led2_timer >= led2_stim_time) {
      analogWrite (led2_pin, 0);
      led2_state = LOW;
      led2_last_change = currentMillis;
    }
  }
  else{ //led2 state is off
    if (led2_timer >= led2_off_time) {
      analogWrite (led2_pin, led2_brightness);
      led2_state = HIGH;
      led2_last_change = currentMillis;
    }
  }
  led3_timer = currentMillis - led3_last_change;
  if (led3_state = = HIGH);
    if (led3_timer >= led3_stim_time) {
      analogWrite(led3_pin, 0);
      led3_state = LOW;
      led3_last_change = currentMillis;
    }
  }
  else{ //led3 state is off
  if (led3_timer >= led3_off_time) {
      analogWrite(led3_pin, led3_brightness);
      led3_state = HIGH;
      led3_last_change = currentMillis;
    }
  }
  led4_timer = currentMillis - led4_last_change;
  if (led4_state = = HIGH);
    if (led4_timer >= led4_stim_time) {
      analogWrite(led4_pin, 0);
      led4_state = LOW;
      led4_last_change = currentMillis;
    }
  }
  else{ //led4 state is off
    if (led4Jimer >= led4_off_time) {
      analogWrite(led4_pin, led4_brightness);
      led4_state = HIGH;
      led4_last_change = currentMillis;
    }
  }
  led5_timer = currentMillis - led5_last_change;
  if (led5_state = = HIGH);
    if (led5_timer >= led5_stim_time) {
      analogWrite(led5_pin, 0);
      led5_state = LOW;
      led5_last_change = currentMillis;
    }
  }
  else{ //led5 state is off
    if (led5_timer >= led5_off_time) {
      analogWrite(led5_pin, led5_brightness);
      led5_state = HIGH;
      led5_last_change = currentMillis;
    }
  }
  led6_timer = currentMillis - led6_last_change;
  if (led6_state = = HIGH);
    if (led6_timer >= led6_stim_time) {
      analogWrite(led6_pin, 0);
      led6_state = LOW;
      led6_last_change = currentMillis;
    }
  }
  else{ //led6 state is off
    if (led6_timer >= led6_off_time) {
      analogWrite(led6_pin, led6_brightness);
      led6_state = HIGH;
      led6_last_change = currentMillis;
    }
  }
}
```

### Example 12

### Optical Control of Endogenous Mammalian Transcription

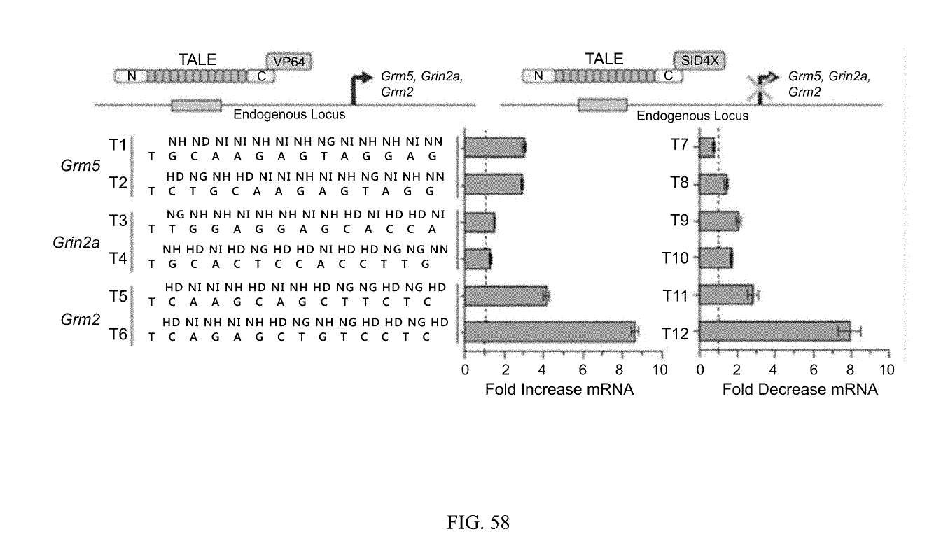

To test the efficacy of AAV-mediated TALE delivery for modulating transcription in primary mouse cortical neurons, Applicants constructed six TALE-DNA binding domains targeting the genetic loci of three mouse neurotransmitter receptors: Grm5, Grin2a, and Grm2, which encode mGluR5, NMDA subunit 2A and mGluR2, respectively (FIG. 58). To increase the likelihood of a target site accessibility, Applicants used mouse cortex DNase I sensitivity data from the UCSC genome browser to identify putative open chromatin regions. DNase I sensitive regions in the promoter of each target gene provided a guide for the selection of TALE binding sequences (FIG. 46). For each TALE, Applicants employed VP64 as a transcriptional activator or a quadruple tandem repeat of the mSin3 interaction domain (SID) (Beerli, R. R., Segal, D. J., Dreier, B. & Barbas, C. F., 3rd Toward controlling gene expression at will: specific regulation of the erbB-2/HER-2 promoter by using polydactyl zinc finger proteins constructed from modular building blocks. Proc Natl Acad Sci USA 95, 14628-14633 (1998) and Ayer, D. E., Laherty, C.D., Lawrence, Q. A., Armstrong, A. P. & Eisenman, R.N. Mad proteins contain a dominant transcription repression domain. Molecular and Cellular Biology 16, 5772-5781 (1996)) as a repressor. Applicants have previously shown that a single SID fused to TALE downregulated a target gene effectively in 293FT cells (Cong, L., Zhou, R., Kuo, Y.-c., Cunniff, M. & Zhang, F. Comprehensive interrogation of natural TALE DNA-binding modules and transcriptional repressor domains. Nat Commun 3, 968 (2012)). Hoping to further improve this TALE repressor, Applicants reasoned that four repeats of SID—analogous to the successful quadruple VP16 repeat architecture of VP64 (Beerli, R. R., Segal, D. J., Dreier, B. & Barbas, C.F., 3rd Toward controlling gene expression at will: specific regulation of the erbB-2/HER-2 promoter by using polydactyl zinc finger proteins constructed from modular building blocks. Proc Natl Acad Sci USA 95, 14628-14633 (1998)—might augment its repressive activity. This was indeed the case, as TALE-SID4X constructs enhanced repression ˜2-fold over TALE-SID in 293FT cells (FIG. 54).

Applicants found that four out of six TALE-VP64 constructs (T1, T2, T5 and T6) efficiently activated their target genes Grm5 and Grm2 in AAV-transduced primary neurons by up to 3- and 8-fold, respectively (FIG. 58). Similarly, four out of six TALE-SID4X repressors (T9, T10, T11, T12) reduced the expression of their endogenous targets Grin2a and Grm2 by up to 2- and 8-fold, respectively (FIG. 58). Together, these results indicate that constitutive TALEs can positively or negatively modulate endogenous target gene expression in neurons. Notably, efficient activation or repression by a given TALE did not predict its efficiency at transcriptional modulation in the opposite direction. Therefore, multiple TALEs may need to be screened to identify the most effective TALE for a particular locus.

For a neuronal application of LITEs, Applicants selected the Grm2 TALE (T6), which exhibited the strongest level of target upregulation in primary neurons, based on Applicants' comparison of 6 constitutive TALE activators (FIG. 58). Applicants investigated its function using 2 light pulsing frequencies with the same duty cycle of 0.8%. Both stimulation conditions achieved a ˜7-fold light-dependent increase in Grm2 mRNA levels (FIG. 38C). Further study confirmed that, significant target gene expression increases could be attained quickly (4-fold upregulation within 4 h; FIG. 38D). In addition, Applicants observed significant upregulation of mGluR2 protein after stimulation, demonstrating that changes effected by LITEs at the mRNA level are translated to the protein domain (FIG. 38E). Taken together, these results confirm that LITEs enable temporally precise optical control of endogenous gene expression in neurons.

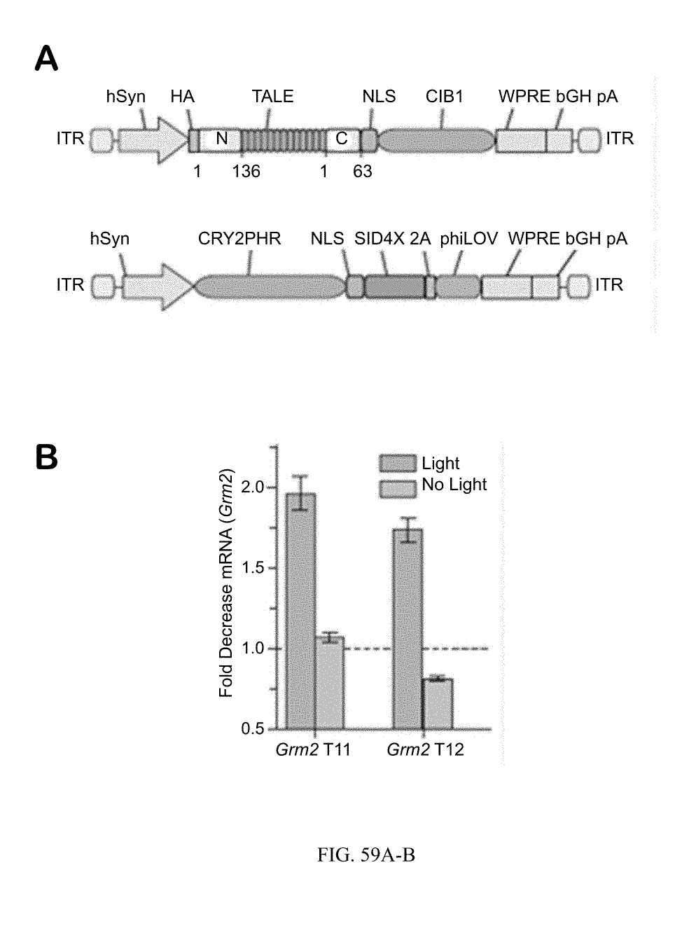

As a compliment to Applicants' previously implemented LITE activators, Applicants next engineered a LITE repressor based on the TALE-SID4X constructs. Constitutive Grm2 TALEs (T11 and T12, FIG. 59A) mediated the highest level of transcription repression, and were chosen as LITE repressors (FIG. 59A, B). Both light-induced repressors mediated significant downregulation of Grm2 expression, with 1.95-fold and 1.75-fold reductions for T11 and T12, respectively, demonstrating the feasibility of optically controlled repression in neurons (FIG. 38G).

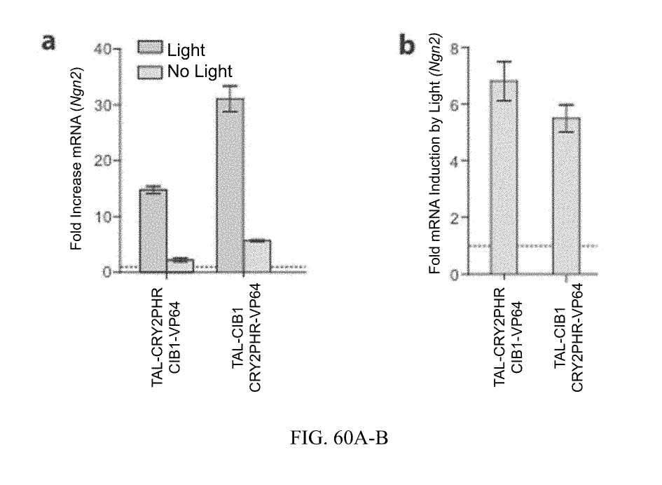

In order to deliver LITEs into neurons using AAV, Applicants had to ensure that the total viral genome size, with the LITE transgenes included, did not exceed 4.8 kb (Wu, Z., Yang, H. & Colosi, P. Effect of Genome Size on AAV Vector Packaging. Mol Ther 18, 80-86 (2009) and Dong JY, F. P., Frizzell R A Quantitative analysis of the packaging capacity of recombinant adeno-associated virus. Human Gene Therapy 7, 2101-2112 (1996)). To that end, Applicants shortened the TALE N- and C-termini (keeping 136 aa in the N-terminus and 63 aa in the C-terminus) and exchanged the CRY2PHR and CIB1 domains (TALE-CIB1 and CRY2PHR-VP64; FIG. 38A). This switch allowed each component of LITE to fit into AAV vectors and did not reduce the efficacy of light-mediated transcription modulation (FIG. 60). These LITEs can be efficiently delivered into primary cortical neurons via co-transduction by a combination of two AAV vectors (FIG. 38B; delivery efficiencies of 83-92% for individual components with >80% co-transduction efficiency).

### Example 13

### Inducible Lentiviral Cas9

Lentivirus preparation. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media was changed to OptiMEM (serum-free) media and transfection was done 4 hours later. Cells were transfected with 10ug of lentiviral transfer plasmid (pCasES10) and the following packaging plasmids: 5ug of pMD2.G (VSV-g pseudotype), and 7.5ug of psPAX2 (gag/pol/rev/tat). Transfection was done in 4 mL OptiMEM with a cationic lipid delivery agent (50 uL Lipofectamine 2000 and 100ul Plus reagent). After 6 hours, the media was changed to antibiotic-free DMEM with 10% fetal bovine serum.

Lentivirus purification. Viral supernatants were harvested after 48 hours. Supernatants were first cleared of debris and filtered through a 0.45 um low protein binding (PVDF) filter. They were then spun in a ultracentrifuge for 2 hours at 24,000 rpm. Viral pellets were resuspended in 50 ul of DMEM overnight at 4 C. They were then aliquotted and immediately frozen at −80 C.

Clonal isolation using FACS. For clonal isolation of HEK293FT and HUES64 human embryonic stem cells, cells were infected in suspension with either 1ul or 5ul of purified virus. Twenty-four hours post infection, 1uM doxycycline was added to the cell culture media. After 24 or 48 hours more, cells underwent fluorescence-assisted cell sorting (FACS ) on a BD FACSAria IIu instrument to isolate single cells that robustly expressed EGFP (and hence Cas9) after doxycycline treatment. Cell were plated either in bulk or into individual wells to allow selection of clonal populations with an integrated inducible Cas9 for further use. Sort efficiency was always >95% and cells were visualized immediately after plating to verify EGFP fluorescence.

FIG. 61 depicts Tet Cas9 vector designs

FIG. 62 depicts a vector and EGFP expression in 293FT cells.

Sequence of pCasES020 inducible Cas9:

(SEQ ID NO: 211)

caactttgtatagaaaagttggctccgaattcgcccttcaggtccgaggt
tctagacgagtttactccctatcagtgatagagaacgatgtcgagtttac
tccctatcagtgatagagaacgtatgtcgagtttactccctatcagtgat
agagaacgtatgtcgagtttactccctatcagtgatagagaacgtatgtc
gagtttatccctatcagtgatagagaacgtatgtcgagtttactccctat
cagtgatagagaacgtatgtcgaggtaggcgtgtacggtgggaggcctat
ataagcagagctcgtttagtgaaccgtcagatcgcaaagggcgaattcga
cccaagtttgtacagccaccATGGACTATAAGGACCACGACGGAGACTAC
AAGGATCATGATATTGATTACAAAGACGATGACGATAAGATGGCCCCAAA
GAAGAAGCGGAAGGTCGGTATCCACGGAGTCCCAGCAGCCGACAAGAAGT
ACAGCATCGGCCTGGACATCGGCACCAACTCTGTGGGCTGGGCCGTGATC
ACCGACGAGTACAAGGTGCCCAGCAAGAAATTCAAGGTGCTGGGCAACAC
CGACCGGCACAGCATCAAGAAGAACCTGATCGGAGCCCTGCTGTTCGACA
GCGGCGAAACAGCCGAGGCCACCCGGCTGAAGAGAACCGCCAGAAGAAGA
TACACCAGACGGAAGAACCGGATCTGCTATCTGCAAGAGATCTTCAGCAA
CGAGATGGCCAAGGTGGACGACAGCTTCTTCCACAGACTGGAAGAGTCCT
TCCTGGTGGAAGAGGATAAGAAGCACGAGCGGCACCCCATCTTCGGCAAC
ATCGTGGACGAGGTGGCCTACCACGAGAAGTACCCCACCATCTACCACCT
GAGAAAGAAACTGGTGGACAGCACCGACAAGGCCGACCTGCGGCTGATCT
ATCTGGCCCTGGCCCACATGATCAAGTTCCGGGGCCACTTCCTGATCGAG
GGCGACCTGAACCCCGACAACAGCGACGTGGACAAGCTGTTCATCCAGCT
GGTGCAGACCTACAACCAGCTGTTCGAGGAAAACCCCATCAACGCCAGCG
GCGTGGACGCCAAGGCCATCCTGTCTGCCAGACTGAGCAAGAGCAGACGG
CTGGAAAATCTGATCGCCCAGCTGCCCGGCGAGAAGAAGAATGGCCTGTT
CGGCAACCTGATTGCCCTGAGCCTGGGCCTGACCCCCAACTTCAAGAGCA
ACTTCGACCTGGCCGAGGATGCCAAACTGCAGCTGAGCAAGGACACCTAC
GACGACGACCTGGACAACCTGCTGGCCCAGATCGGCGACCAGTACGCCGA
CCTGTTTCTGGCCGCCAAGAACCTGTCCGACGCCATCCTGCTGAGCGACA
TCCTGAGAGTGAACACCGAGATCACCAAGGCCCCCCTGAGCGCCTCTATG
ATCAAGAGATACGACGAGCACCACCAGGACCTGACCCTGCTGAAAGCTCT
CGTGCGGCAGCAGCTGCCTGAGAAGTACAAAGAGATTTTCTTCGACCAGA
GCAAGAACGGCTACGCCGGCTACATTGACGGCGGAGCCAGCCAGGAAGAG
TTCTACAAGTTCATCAAGCCCATCCTGGAAAAGATGGACGGCACCGAGGA
ACTGCTCGTGAAGCTGAACAGAGAGGACCTGCTGCGGAAGCAGCGGACCT
TCGACAACGGCAGCATCCCCCACCAGATCCACCTGGGAGAGCTGCACGCC
ATTCTGCGGCGGCAGGAAGATTTTTACCCATTCCTGAAGGACAACCGGGA
AAAGATCGAGAAGATCCTGACCTTCCGCATCCCCTACTACGTGGGCCCTC
TGGCCAGGGGAAACAGCAGATTCGCCTGGATGACCAGAAAGAGCGAGGAA
ACCATCACCCCCTGGAACTTCGAGGAAGTGGTGGACAAGGGCGCTTCCGC
CCAGAGCTTCATCGAGCGGATGACCAACTTCGATAAGAACCTGCCCAACG
AGAAGGTGCTGCCCAAGCACAGCCTGCTGTACGAGTACTTCACCGTGTAT
AACGAGCTGACCAAAGTGAAATACGTGACCGAGGGAATGAGAAAGCCCGC
CTTCCTGAGCGGCGAGCAGAAAAAGGCCATCGTGGACCTGCTGTTCAAGA
CCAACCGGAAAGTGACCGTGAAGCAGCTGAAAGAGGACTACTTCAAGAAA
ATCGAGTGCTTCGACTCCGTGGAAATCTCCGGCGTGGAAGATCGGTTCAA
CGCCTCCCTGGGCACATACCACGATCTGCTGAAAATTATCAAGGACAAGG
ACTTCCTGGACAATGAGGAAAACGAGGACATTCTGGAAGATATCGTGCTG
ACCCTGACACTGTTTGAGGACAGAGAGATGATCGAGGAACGGCTGAAAAC
CTATGCCCACCTGTTCGACGACAAAGTGATGAAGCAGCTGAAGCGGCGGA
GATACACCGGCTGGGGCAGGCTGAGCCGGAAGCTGATCAACGGCATCCGG
GACAAGCAGTCCGGCAAGACAATCCTGGATTTCCTGAAGTCCGACGGCTT
CGCCAACAGAAACTTCATGCAGCTGATCCACGACGACAGCCTGACCTTTA
AAGAGGACATCCAGAAAGCCCAGGTGTCCGGCCAGGGCGATAGCCTGCAC
GAGCACATTGCCAATCTGGCCGGCAGCCCCGCCATTAAGAAGGGCATCCT
GCAGACAGTGAAGGTGGTGGACGAGCTCGTGAAAGTGATGGGCCGGCACA
AGCCCGAGAACATCGTGATCGAAATGGCCAGAGAGAACCAGACCACCCAG
AAGGGACAGAAGAACAGCCGCGAGAGAATGAAGCGGATCGAAGAGGGCAT
CAAAGAGCTGGGCAGCCAGATCCTGAAAGAACACCCCGTGGAAAACACCC
AGCTGCAGAACGAGAAGCTGTACCTGTACTACCTGCAGAATGGGCGGGAT
ATGTACGTGGACCAGGAACTGGACATCAACCGGCTGTCCGACTACGATGT
GGACCATATCGTGCCTCAGAGCTTTCTGAAGGACGACTCCATCGACAACA
AGGTGCTGACCAGAAGCGACAAGAACCGGGGCAAGAGCGACAACGTGCCC
TCCGAAGAGGTCGTGAAGAAGATGAAGAACTACTGGCGGCAGCTGCTGAA
CGCCAAGCTGATTACCCAGAGAAAGTTCGACAATCTGACCAAGGCCGAGA
GAGGCGGCCTGAGCGAACTGGATAAGGCCGGCTTCATCAAGAGACAGCTG
GTGGAAACCCGGCAGATCACAAAGCACGTGGCACAGATCCTGGACTCCCG
GATGAACACTAAGTACGACGAGAATGACAAGCTGATCCGGGAAGTGAAAG
TGATCACCCTGAAGTCCAAGCTGGTGTCCGATTTCCGGAAGGATTTCCAG
TTTTACAAAGTGCGCGAGATCAACAACTACCACCACGCCCACGACGCCTA
CCTGAACGCCGTCGTGGGAACCGCCCTGATCAAAAAGTACCCTAAGCTGG
AAAGCGAGTTCGTGTACGGCGACTACAAGGTGTACGACGTGCGGAAGATG
ATCGCCAAGAGCGAGCAGGAAATCGGCAAGGCTACCGCCAAGTACTTCTT
CTACAGCAACATCATGAACTTTTTCAAGACCGAGATTACCCTGGCCAACG
GCGAGATCCGGAAGCGGCCTCTGATCGAGACAAACGGCGAAACCGGGGAG
ATCGTGTGGGATAAGGGCCGGGATTTTGCCACCGTGCGGAAAGTGCTGAG
CATGCCCCAAGTGAATATCGTGAAAAAGACCGAGGTGCAGACAGGCGGCT
TCAGCAAAGAGTCTATCCTGCCCAAGAGGAACAGCGATAAGCTGATCGCC
AGAAAGAAGGACTGGGACCCTAAGAAGTACGGCGGCTTCGACAGCCCCAC
CGTGGCCTATTCTGTGCTGGTGGTGGCCAAAGTGGAAAAGGGCAAGTCCA
AGAAACTGAAGAGTGTGAAAGAGCTGCTGGGGATCACCATCATGGAAAGA
AGCAGCTTCGAGAAGAATCCCATCGACTTTCTGGAAGCCAAGGGCTACAA
AGAAGTGAAAAAGGACCTGATCATCAAGCTGCCTAAGTACTCCCTGTTCG
AGCTGGAAAACGGCCGGAAGAGAATGCTGGCCTCTGCCGGCGAACTGCAG
AAGGGAAACGAACTGGCCCTGCCCTCCAAATATGTGAACTTCCTGTACCT
GGCCAGCCACTATGAGAAGCTGAAGGGCTCCCCCGAGGATAATGAGCAGA
AACAGCTGTTTGTGGAACAGCACAAGCACTACCTGGACGAGATCATCGAG
CAGATCAGCGAGTTCTCCAAGAGAGTGATCCTGGCCGACGCTAATCTGGA
CAAAGTGCTGTCCGCCTACAACAAGCACCGGGATAAGCCCATCAGAGAGC
AGGCCGAGAATATCATCCACCTGTTTACCCTGACCAATCTGGGAGCCCCT
GCCGCCTTCAAGTACTTTGACACCACCATCGACCGGAAGAGGTACACCAG
CACCAAAGAGGTGCTGGACGCCACCCTGATCCACCAGAGCATCACCGGCC
TGTACGAGACACGGATCGACCTGTCTCAGCTGGGAGGCGACAAAAGGCCG
GCGGCCACGAAAAAGGCCGGCCAGGCAAAAAAGAAAAAGgaattctctag
aGGCAGTGGAGAGGGCAGAGGAAGTCTGCTAACATGCGGTGACGTCGAGG
AGAATCCTGGCCCAGTGAGCAAGGGCGAGGAGCTGTTCACCGGGGTGGTG
CCCATCCTGGTCGAGCTGGACGGCGACGTAAACGGCCACAAGTTCAGCGT
GTCCGGCGAGGGCGAGGGCGATGCCACCTACGGCAAGCTGACCCTGAAGT
TCATCTGCACCACCGGCAAGCTGCCCGTGCCCTGGCCCACCCTCGTGACC
ACCCTGACCTACGGCGTGCAGTGCTTCAGCCGCTACCCCGACCACATGAA
GCAGCACGACTTCTTCAAGTCCGCCATGCCCGAAGGCTACGTCCAGGAGC
GCACCATCTTCTTCAAGGACGACGGCAACTACAAGACCCGCGCCGAGGTG
AAGTTCGAGGGCGACACCCTGGTGAACCGCATCGAGCTGAAGGGCATCGA
CTTCAAGGAGGACGGCAACATCCTGGGGCACAAGCTGGAGTACAACTACA
ACAGCCACAACGTCTATATCATGGCCGACAAGCAGAAGAACGGCATCAAG
GTGAACTTCAAGATCCGCCACAACATCGAGGACGGCAGCGTGCAGCTCGC
CGACCACTACCAGCAGAACACCCCCATCGGCGACGGCCCCGTGCTGCTGC
CCGACAACCACTACCTGAGCACCCAGTCCGCCCTGAGCAAAGACCCCAAC
GAGAAGCGCGATCACATGGTCCTGCTGGAGTTCGTGACCGCCGCCGGGAT
CACTCTCGGCATGGACGAGCTGTACAAGCTCGAGGGAAGCGGAGCTACTA
ACTTCAGCCTGCTGAAGCAGGCTGGCGACGTGGAGGAGAACCCTGGACCT
atgtctaggctggacaagagcaaagtcataaacggagctctggaattact
caatggtgtcggtatcgaaggcctgacgacaaggaaactcgctcaaaagc
tgggagttgagcagcctaccctgtactggcacgtgaagaacaagcgggcc
ctgctcgatgccctgccaatcgagatgctggacaggcatcatacccactt
ctgccccctggaaggcgagtcatggcaagactttctgcggaacaacgcca
agtcataccgctgtgctctcctctcacatcgcgacggggctaaagtgcat
ctcggcacccgcccaacagagaaacagtacgaaaccctggaaaatcagct
cgcgttcctgtgtcagcaaggcttctccctggagaacgcactgtacgctc
tgtccgccgtgggccactttacactgggctgcgtattggaggaacaggag
catcaagtagcaaaagaggaaagagagacacctaccaccgattctatgcc
cccacttctgagacaagcaattgagctgttcgaccggcagggagccgaac
ctgccttccttttcggcctggaactaatcatatgtggcctggagaaacag
ctaaagtgcgaaagcggcgggccgaccgacgcccttgacgattttgactt
agacatgctcccagccgatgcccttgacgattttgaccttgacatgctcc
ccgggtaatgtacaaagtggtgaattccggcaattcgatatcaagcttat
cgataatcaacctctggattacaaaatttgtgaaagattgactggtattc
ttaactatgttgctccttttacgctatgtggatacgctgctttaatgcct
ttgtatcatgctattgcttcccgtatggctttcattttctcctccttgta
taaatcctggttgctgtctctttatgaggagttgtggcccgttgtcaggc
aacgtggcgtggtgtgcactgtgtttgctgacgcaacccccactggttgg
ggcattgccaccacctgtcagctcctttccgggactttcgctttccccct
ccctattgccacggcggaactcatcgccgcctgccttgcccgctgctgga
caggggctcggctgttgggcactgacaattccgtggtgttgtcggggaaa
tcatcgtcctttccttggctgctcgcctgtgttgccacctggattctgcg
cgggacgtccttctgctacgtcccttcggccctcaatccagcggaccttc
cttcccgcggcctgctgccggctctgcggcctcttccgcgtcttcgcctt
cgccctcagacgagtcggatctccctttgggccgcctccccgcatcgata
ccgtcgacctcgagacctagaaaaacatggagcaatcacaagtagcaata
cagcagctaccaatgctgattgtgcctggctagaagcacaagaggaggag
gaggtgggttttccagtcacacctcaggtacctttaagaccaatgactta
caaggcagctgtagatcttagccactttttaaaagaaaaggggggactgg
aagggctaattcactcccaacgaagacaagatatccttgatctgtggatc
taccacacacaaggctacttccctgattggcagaactacacaccagggcc
agggatcagatatccactgacctttggatggtgctacaagctagtaccag
ttgagcaagagaaggtagaagaagccaatgaaggagagaacacccgcttg
ttacaccctgtgagcctgcatgggatggatgacccggagagagaagtatt
agagtggaggtttgacagccgcctagcatttcatcacatggcccgagagc
tgcatccggactgtactgggtctctctggttagaccagatctgagcctgg
gagctctctggctaactagggaacccactgcttaagcctcaataaagctt
gccttgagtgcttcaagtagtgtgtgcccgtctgttgtgtgactctggta
actagagatccctcagacccttttagtcagtgtggaaaatctctagcagg
gcccgtttaaacccgctgatcagcctcgactgtgccttctagttgccagc
catctgttgtttgcccctcccccgtgccttccttgaccctggaaggtgcc
actcccactgtcctttcctaataaaatgaggaaattgcatcgcattgtct
gagtaggtgtcattctattctggggggtggggtggggcaggacagcaagg
gggaggattgggaagacaatagcaggcatgctggggatgcggtgggctct
atggcttctgaggcggaaagaaccagctggggctctagggggtatcccca
cgcgccctgtagcggcgcattaagcgcggcgggtgtggtggttacgcgca
gcgtgaccgctacacttgccagcgccctagcgcccgctcctttcgctttc
ttcccttcctttctcgccacgttcgccggctttccccgtcaagctctaaa
tcgggggctccctttagggttccgatttagtgctttacggcacctcgacc
ccaaaaaacttgattagggtgatggttcacgtagtgggccatcgccctga
tagacggtttttcgccctttgacgttggagtccacgttctttaatagtgg
actcttgttccaaactggaacaacactcaaccctatctcggtctattctt
ttgatttataagggattttgccgatttcggcctattggttaaaaaatgag
ctgatttaacaaaaatttaacgcgaattaattctgtggaatgtgtgtcag
ttagggtgtggaaagtccccaggctccccagcaggcagaagtatgcaaag
catgcatctcaattagtcagcaaccaggtgtggaaagtccccaggctccc
cagcaggcagaagtatgcaaagcatgcatctcaattagtcagcaaccata
gtcccgcccctaactccgcccatcccgcccctaactccgcccagttccgc
ccattctccgccccatggctgactaattttttttatttatgcagaggccg
aggccgcctctgcctctgagctattccagaagtagtgaggaggctttttt
ggaggcctaggcttttgcaaaaagctcccgggagcttgtatatccatttt
cggatctgatcagcacgtgttgacaattaatcatcggcatagtatatcgg
catagtataatacgacaaggtgaggaactaaaccatggccaagttgacca
gtgccgttccggtgctcaccgcgcgcgacgtcgccggagcggtcgagttc
tggaccgaccggctcgggttctcccgggacttcgtggaggacgacttcgc
cggtgtggtccgggacgacgtgaccctgttcatcagcgcggtccaggacc
aggtggtgccggacaacaccctggcctgggtgtgggtgcgcggcctggac
gagctgtacgccgagtggtcggaggtcgtgtccacgaacttccgggacgc
ctccgggccggccatgaccgagatcggcgagcagccgtgggggcgggagt
tcgccctgcgcgacccggccggcaactgcgtgcacttcgtggccgaggag
caggactgacacgtgctacgagatttcgattccaccgccgccttctatga
aaggttgggcttcggaatcgttttccgggacgccggctggatgatcctcc
agcgcggggatctcatgctggagttcttcgcccaccccaacttgtttatt
gcagcttataatggttacaaataaagcaatagcatcacaaatttcacaaa
taaagcatttttttcactgcattctagttgtggtttgtccaaactcatca
atgtatcttatcatgtctgtataccgtcgacctctagctagagcttggcg
taatcatggtcatagctgtttcctgtgtgaaattgttatccgctcacaat
tccacacaacatacgagccggaagcataaagtgtaaagcctggggtgcct
aatgagtgagctaactcacattaattgcgttgcgctcactgcccgctttc
cagtcgggaaacctgtcgtgccagctgcattaatgaatcggccaacgcgc
ggggagaggcggtttgcgtattgggcgctcttccgcttcctcgctcactg
actcgctgcgctcggtcgttcggctgcggcgagcggtatcagctcactca
aaggcggtaatacggttatccacagaatcaggggataacgcaggaaagaa
catgtgagcaaaaggccagcaaaaggccaggaaccgtaaaaaggccgcgt
tgctggcgtttttccataggctccgcccccctgacgagcatcacaaaaat
cgacgctcaagtcagaggtggcgaaacccgacaggactataaagatacca
ggcgtttccccctggaagctccctcgtgcgctctcctgttccgaccctgc
cgcttaccggatacctgtccgcctttctcccttcgggaagcgtggcgctt
tctcatagctcacgctgtaggtatctcagttcggtgtaggtcgttcgctc
caagctgggctgtgtgcacgaaccccccgttcagcccgaccgctgcgcct
tatccggtaactatcgtcttgagtccaacccggtaagacacgacttatcg
ccactggcagcagccactggtaacaggattagcagagcgaggtatgtagg
cggtgctacagagttcttgaagtggtggcctaactacggctacactagaa
gaacagtatttggtatctgcgctctgctgaagccagttaccttcggaaaa
agagttggtagctcttgatccggcaaacaaaccaccgctggtagcggtgg
tttttttgtttgcaagcagcagattacgcgcagaaaaaaaggatctcaag
aagatcctttgatcttttctacggggtctgacgctcagtggaacgaaaac
tcacgttaagggattttggtcatgagattatcaaaaaggatcttcaccta
gatccttttaaattaaaaatgaagttttaaatcaatctaaagtatatatg
agtaaacttggtctgacagttaccaatgcttaatcagtgaggcacctatc
tcagcgatctgtctatttcgttcatccatagttgcctgactccccgtcgt
gtagataactacgatacgggagggcttaccatctggccccagtgctgcaa
tgataccgcgagacccacgctcaccggctccagatttatcagcaataaac
cagccagccggaagggccgagcgcagaagtggtcctgcaactttatccgc
ctccatccagtctattaattgttgccgggaagctagagtaagtagttcgc
cagttaatagtttgcgcaacgttgttgccattgctacaggcatcgtggtg
tcacgctcgtcgtttggtatggcttcattcagctccggttcccaacgatc
aaggcgagttacatgatcccccatgttgtgcaaaaaagcggttagctcct
tcggtcctccgatcgttgtcagaagtaagttggccgcagtgttatcactc
atggttatggcagcactgcataattctcttactgtcatgccatccgtaag
atgcttttctgtgactggtgagtactcaaccaagtcattctgagaatagt
gtatgcggcgaccgagttgctcttgcccggcgtcaatacgggataatacc
gcgccacatagcagaactttaaaagtgctcatcattggaaaacgttcttc
ggggcgaaaactctcaaggatcttaccgctgttgagatccagttcgatgt
aacccactcgtgcacccaactgatcttcagcatcttttactttcaccagc
gtttctgggtgagcaaaaacaggaaggcaaaatgccgcaaaaaagggaat
aagggcgacacggaaatgttgaatactcatactcttcctttttcaatatt
attgaagcatttatcagggttattgtctcatgagcggatacatatttgaa
tgtatttagaaaaataaacaaataggggttccgcgcacatttccccgaaa
agtgccacctgacgtcgacggatcgggagatctcccgatcccctatggtg
cactctcagtacaatctgctctgatgccgcatagttaagccagtatctgc
tccctgcttgtgtgttggaggtcgctgagtagtgcgcgagcaaaatttaa
gctacaacaaggcaaggcttgaccgacaattgcatgaagaatctgcttag
ggttaggcgttttgcgctgcttcgcgatgtacgggccagatatacgcgtt
gacattgattattgactagttattaatagtaatcaattacggggtcatta
gttcatagcccatatatggagttccgcgttacataacttacggtaaatgg
cccgcctggctgaccgcccaacgacccccgcccattgacgtcaataatga
cgtatgttcccatagtaacgccaatagggactttccattgacgtcaatgg
gtggagtatttacggtaaactgcccacttggcagtacatcaagtgtatca
tatgccaagtacgccccctattgacgtcaatgacggtaaatggcccgcct
ggcattatgcccagtacatgaccttatgggactttcctacttggcagtac
atctacgtattagtcatcgctattaccatggtgatgcggttttggcagta
catcaatgggcgtggatagcggtttgactcacggggatttccaagtctcc
accccattgacgtcaatgggagtttgttttggcaccaaaatcaacgggac
tttccaaaatgtcgtaacaactccgccccattgacgcaaatgggcggtag
gcgtgtacggtgggaggtctatataagcagcgcgttttgcctgtactggg
tctctctggttagaccagatctgagcctgggagctctctggctaactagg
gaacccactgcttaagcctcaataaagcttgccttgagtgcttcaagtag
tgtgtgcccgtctgttgtgtgactctggtaactagagatccctcagaccc
ttttagtcagtgtggaaaatctctagcagtggcgcccgaacagggacttg
aaagcgaaagggaaaccagaggagctctctcgacgcaggactcggcttgc
tgaagcgcgcacggcaagaggcgaggggcggcgactggtgagtacgccaa
aaattttgactagcggaggctagaaggagagagatgggtgcgagagcgtc
agtattaagcgggggagaattagatcgcgatgggaaaaaattcggttaag
gccagggggaaagaaaaaatataaattaaaacatatagtatgggcaagca
gggagctagaacgattcgcagttaatcctggcctgttagaaacatcagaa
ggctgtagacaaatactgggacagctacaaccatcccttcagacaggatc
agaagaacttagatcattatataatacagtagcaaccctctattgtgtgc
atcaaaggatagagataaaagacaccaaggaagctttagacaagatagag
gaagagcaaaacaaaagtaagaccaccgcacagcaagcggccgctgatct
tcagacctggaggaggagatatgagggacaattggagaagtgaattatat
aaatataaagtagtaaaaattgaaccattaggagtagcacccaccaaggc
aaagagaagagtggtgcagagagaaaaaagagcagtgggaataggagctt
tgttccttgggttcttgggagcagcaggaagcactatgggcgcagcgtca
atgacgctgacggtacaggccagacaattattgtctggtatagtgcagca
gcagaacaatttgctgagggctattgaggcgcaacagcatctgttgcaac
tcacagtctggggcatcaagcagctccaggcaagaatcctggctgtggaa
agatacctaaaggatcaacagctcctggggatttggggttgctctggaaa
actcatttgcaccactgctgtgccttggaatgctagttggagtaataaat
ctctggaacagatttggaatcacacgacctggatggagtgggacagagaa
attaacaattacacaagcttaatacactccttaattgaagaatcgcaaaa
ccagcaagaaaagaatgaacaagaattattggaattagataaatgggcaa
gtttgtggaattggtttaacataacaaattggctgtggtatataaaatta
ttcataatgatagtaggaggcttggtaggtttaagaatagtttttgctgt
actttctatagtgaatagagttaggcagggatattcaccattatcgtttc
agacccacctcccaaccccgaggggacccgacaggcccgaaggaatagaa
gaagaaggtggagagagagacagagacagatccattcgattagtgaacgg
atcggcactgcgtgcgccaattctgcagacaaatggcagtattcatccac
aattttaaaagaaaaggggggattggggggtacagtgcaggggaaagaat
agtagacataatagcaacagacatacaaactaaagaattacaaaaacaaa
ttacaaaaattcaaaattttcgggtttattacagggacagcagagatcca
gtttggttaattaa

### Example 14: CRISPR Complex Activity in the Nucleus of a Eukaryotic Cell

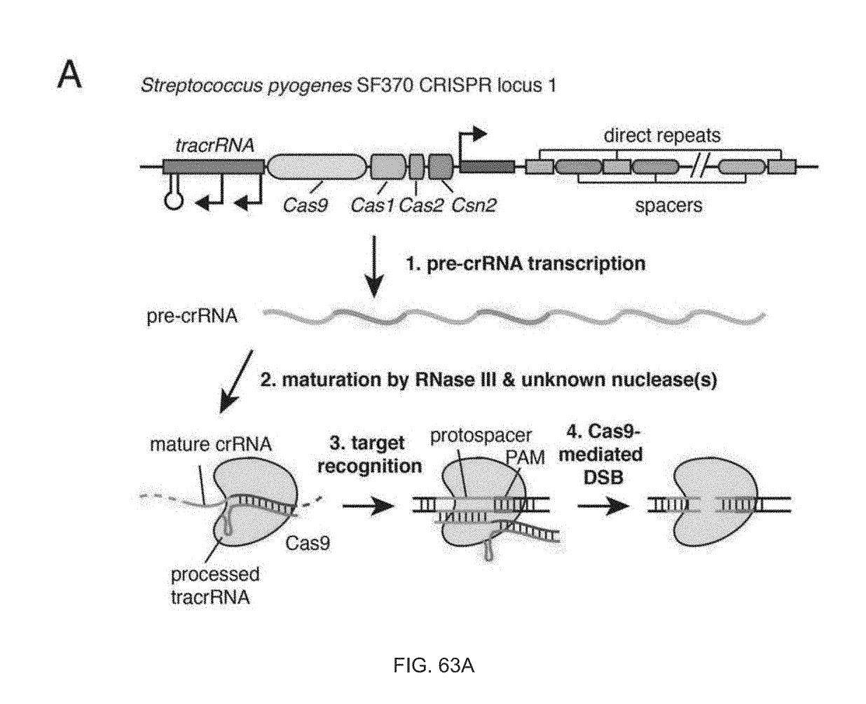

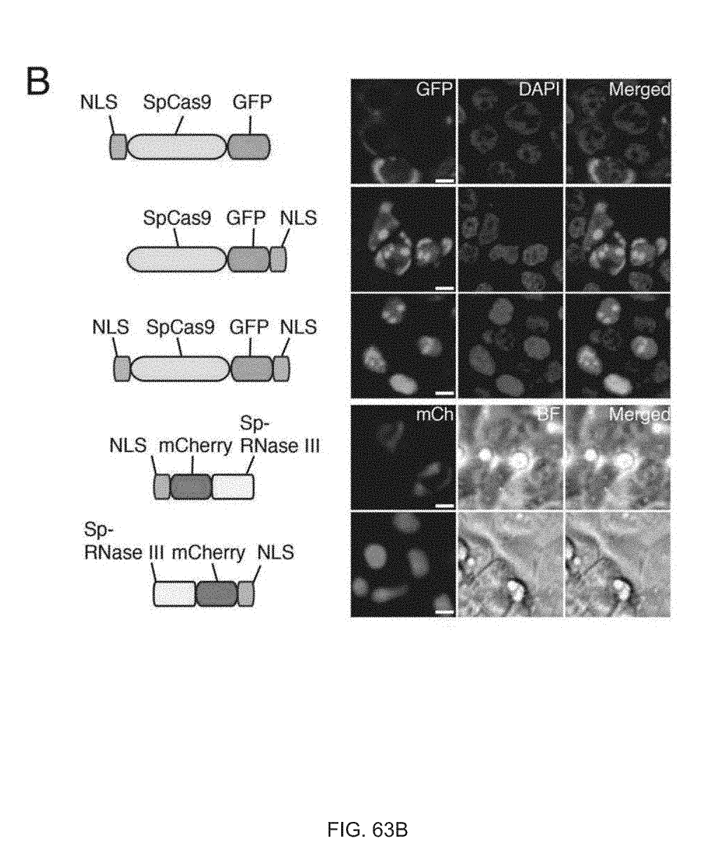

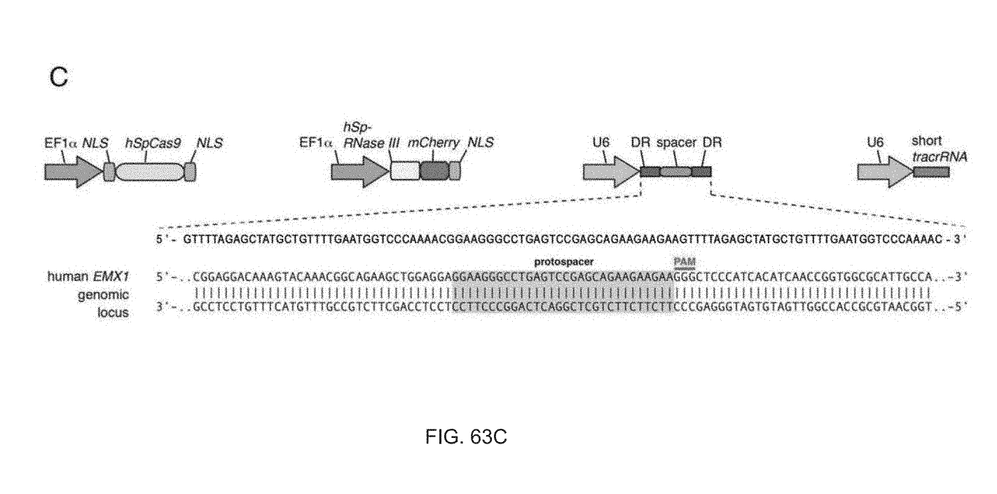

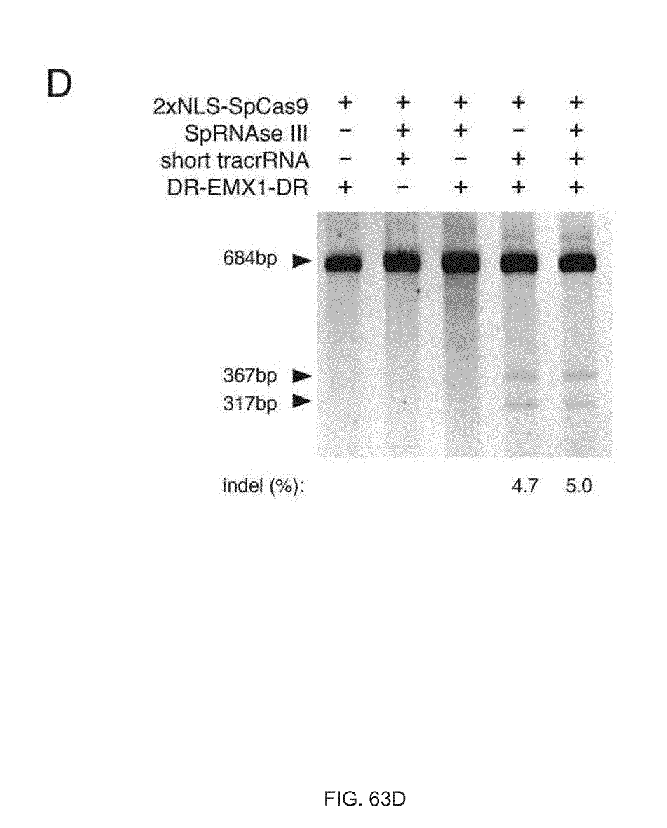

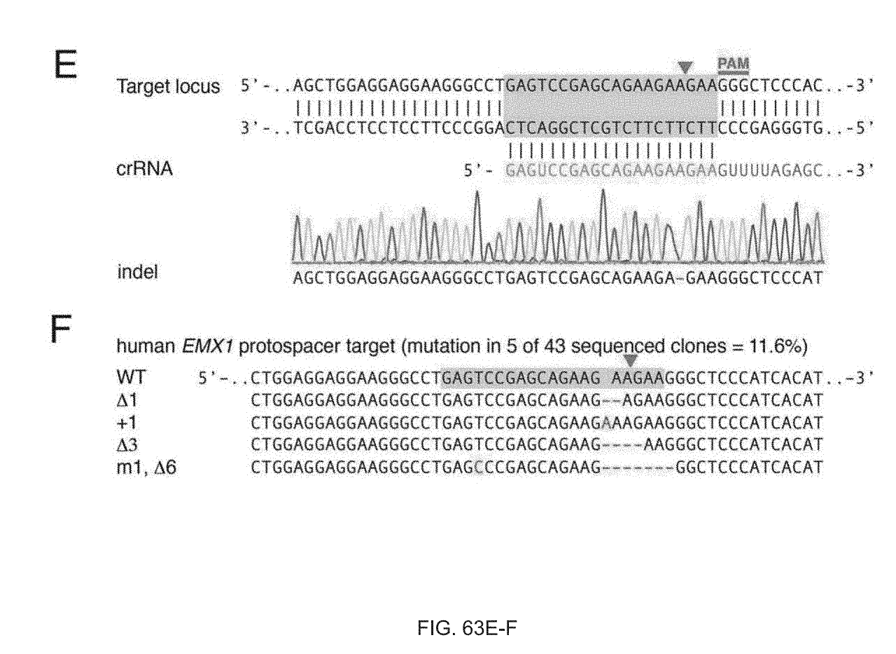

An example type II CRISPR system is the type II CRISPR locus from Streptooccus pyogenes SF370, which contains a cluster of four genes Cas9, Cas1, Cas2, and Csn 1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps (FIG. 63A). First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer (FIG. 63A). This example describes an example process for adapting this RNA-programmable nuclease system to direct CRISPR complex activity in the nuclei of eukaryotic cells.

Cell Culture and Transfection

Human embryonic kidney (HEK) cell line HEK 293FT (Life Technologies) was maintained in Dulbecco's modified Eagle's Medium (DMEM) supplemented with 10% fetal bovine serum (HyClone), 2 mM GlutaMAX (Life Technologies), 100U/mL penicillin, and 100 !lg/mL streptomycin at 3rC with 5% CO2 incubation. Mouse neuro2A (N2A) cell line (ATCC) was maintained with DMEM supplemented with 5% fetal bovine serum (HyClone), 2 mM GlutaMAX (Life Technologies), 100U/mL penicillin, and 100 μg/mL streptomycin at 37° C. with 5% CO2.

HEK 293FT or N2A cells were seeded into 24-well plates (Coming) one day prior to transfection at a density of 200,000 cells per well. Cells were transfected using Lipofectamine 2000 (Life Technologies) following the manufacturer's recommended protocol. For each well of a 24-well plate a total of 800ng of plasmids were used.

Surveyor Assay and Sequencing Analysis for Genome Modification

HEK 293FT or N2A cells were transfected with plasmid DNA as described above. After transfection, the cells were incubated at 37° C. for 72 hours before genomic DNA extraction. Genomic DNA was extracted using the QuickExtract DNA extraction kit (Epicentre) following the manufacturer's protocol. Briefly, cells were resuspended in QuickExtract solution and incubated at 65° C. for 15 minutes and 98° C. for 10 minutes. Extracted genomic DNA was immediately processed or stored at −20° C.

The genomic region surrounding a CRISPR target site for each gene was PCR amplified, and products were purified using QiaQuick Spin Column (Qiagen) following manufacturer's protocol. A total of 400ng of the purified PCR products were mixed with 2 μl 10× Taq polymerase PCR buffer (Enzymatics) and ultrapure water to a final volume of 20 μl, and subjected to are—annealing process to enable heteroduplex formation: 95° C. for 10 min, 95° C. to 85° C. ramping at −2° C./s, 85° C. to 25° C. at −0.25° C./s, and 25° C. hold for 1 minute. After re-annealing, products were treated with Surveyor nuclease and Surveyor enhancer S (Transgenomics) following the manufacturer's recommended protocol, and analyzed on 4-20% Novex TBE poly-acrylamide gels (Life Technologies). Gels were stained with SYBR Gold DNA stain (Life Technologies) for 30 minutes and imaged with a Gel Doc gel imaging system (Bio-rad). Quantification was based on relative band intensities, as a measure of the fraction of cleaved DNA. FIG. 29 provides a schematic illustration of this Surveyor assay.

Restriction Fragment Length Polymorphism Assay for Detection of Homologous Recombination

HEK 293FT and N2A cells were transfected with plasmid DNA, and incubated at 37° C. for 72 hours before genomic DNA extraction as described above. The target genomic region was PCR amplified using primers outside the homology arms of the homologous recombination (HR) template. PCR products were separated on a 1% agarose gel and extracted with MinElute GelExtraction Kit (Qiagen). Purified products were digested with HindIII (Fermentas) and analyzed on a 6% Novex TBE poly-acrylamide gel (Life Technologies).

RNA Secondary Structure Prediction and Analysis

RNA secondary structure prediction was performed using the online webserver RNAfold developed at Institute for Theoretical Chemistty at the University of Vienna, using the centroid structure prediction algorithm (see e.g. A. R. Gruber et al., 2008, Cell 106(1): 23-24; and PA Can and GM Church, 2009, Nature Biotechnology 27(12): 1151-62).

Bacterial Plasmid Transformation Interference Assay

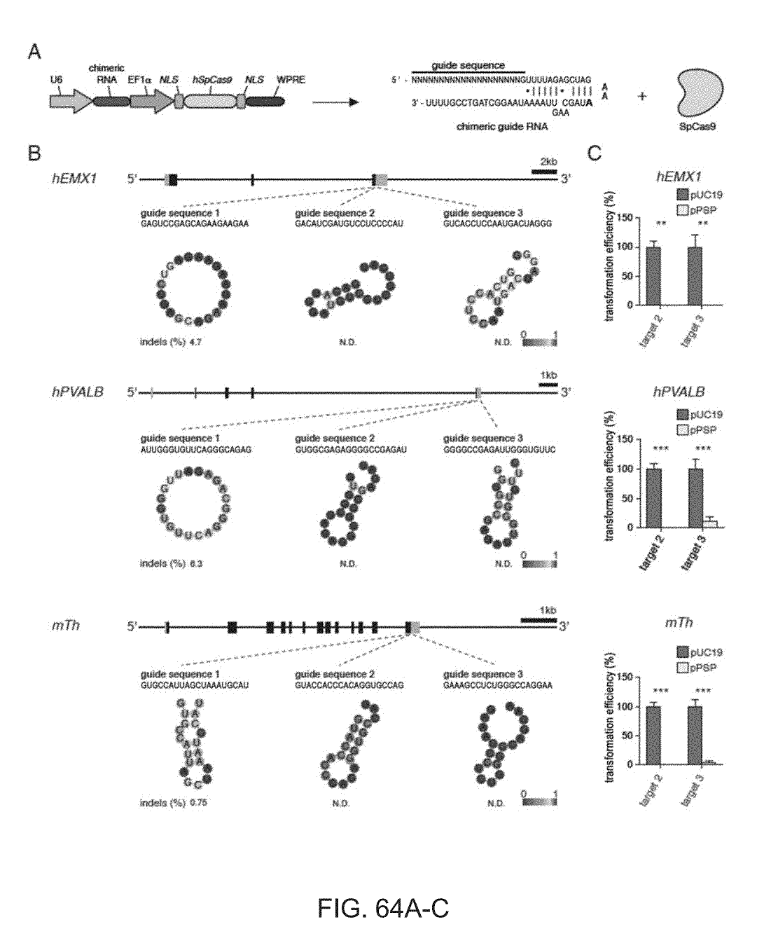

Elements of the S. pyogenes CRISPR locus 1 sufficient for CRISPR activity were reconstituted in E. coli using pCRISPR plasmid (schematically illustrated in FIG. 70A). pCRISPR contained tracrRNA, SpCas9, and a leader sequence driving the crRNA anay. Spacers (also referred to as “guide sequences”) were inserted into the crRNA anay between BsaI sites using annealed oligonucleotides, as illustrated. Challenge plasmids used in the interference assay were constructed by inserting the protospacer (also referred to as a “target sequence”) sequence along with an adjacent CRISPR motif sequence (PAM) into pUC19 (see FIG. 70B). The challenge plasmid contained ampicillin resistance. FIG. 70C provides a schematic representation of the interference assay. Chemically competent E. coli strains already carrying pCRISPR and the appropriate spacer were transformed with the challenge plasmid containing the corresponding protospacer-PAM sequence. pUC19 was used to assess the transformation efficiency of each pCRISPR-carrying competent strain. CRISPR activity resulted in cleavage of the pPSP plasmid carrying the protospacer, precluding ampicillin resistance otherwise conferred by pUC19 lacking the protospacer. FIG. 70D illustrates competence of each pCRISPR-carrying E. coli strain used in assays illustrated in FIG. 64C.

RNA Purification

HEK 293FT cells were maintained and transfected as stated above. Cells were harvested by trypsinization followed by washing in phosphate buffered saline (PBS). Total cell RNA was extracted with TRI reagent (Sigma) following manufacturer's protocol. Extracted total RNA was quantified using Naonodrop (Thermo Scientific) and normalized to same concentration.

Northern Blot Analysis of crRNA and tracrRNA Expression in Mammalian Cells

RNAs were mixed with equal volumes of 2× loading buffer (Ambion), heated to 95° C. for 5 min, chilled on ice for 1 min, and then loaded onto 8% denaturing polyacrylamide gels (SequaGel, National Diagnostics) after pre-running the gel for at least 30 minutes. The samples were electrophoresed for 1.5 hours at 40 W limit. Afterwards, the RNA was transferred to Hybond N+ membrane (GE Healthcare) at 300 rnA in a semi-dry transfer apparatus (Bio-rad) at room temperature for 1.5 hours. The RNA was crosslinked to the membrane using autocrosslink button on Stratagene UV Crosslinker the Stratalinker (Stratagene). The membrane was pre-hybridized in ULTRAhyb-Oligo Hybridization Buffer (Ambion) for 30 min with rotation at 42° C., and probes were then added and hybridized overnight. Probes were ordered from IDT and labeled with [gamma-³²P] ATP (Perkin Elmer) with T4 polynucleotide kinase (New England Biolabs). The membrane was washed once with pre-warmed (42° C.) 2×SSC, 0.5% SDS for 1 min followed by two 30 minute washes at 42° C. The membrane was exposed to a phosphor screen for one hour or overnight at room temperature and then scanned with a phosphorimager (Typhoon).

Bacterial CRISPR System Construction and Evaluation

CRISPR locus elements, including tracrRNA, Cas9, and leader were PCR amplified from Streptococcus pyogenes SF370 genomic DNA with flanking homology arms for Gibson Assembly. Two BsaI type IIS sites were introduced in between two direct repeats to facilitate easy insertion of spacers (FIG. 70). PCR products were cloned into EcoRV-digested pACYC184 downstream of the tet promoter using Gibson Assembly Master Mix (NEB). Other endogenous CRISPR system elements were omitted, with the exception of the last 50 bp of Csn2. Oligos (Integrated DNA Technology) encoding spacers with complimentary overhangs were cloned into the BsaI-digested vector pDC000 (NEB) and then ligated with T7 ligase (Enzymatics) to generate pCRISPR plasmids. Challenge plasmids containing spacers with PAM sequences (also referred to herein as “CRISPR motif sequences”) were created by ligating hybridized oligos carrying compatible overhangs (Integrated DNA Technology) into BamBI-digested pUC19. Cloning for all constructs was performed in E. coli strain JM109 (Zymo Research).

pCRISPR-carrying cells were made competent using the Z-Competent E. coli Transformation Kit and Buffer Set (Zymo Research, T3001) according to manufacturer's instructions. In the transformation assay, 50 uL aliquots of competent cells carrying pCRISPR were thawed on ice and transformed with 1ng of spacer plasmid or pUC19 on ice for 30 minutes, followed by 45 second heat shock at 42° C. and 2 minutes on ice. Subsequently, 250ul SOC (Invitrogen) was added followed by shaking incubation at 37° C. for 1hr, and 100 uL of the post-SOC outgrowth was plated onto double selection plates (12.5 ug/ml chloramphenicol, 100 ug/ml ampicillin). To obtain cfu/ng of DNA, total colony numbers were multiplied by 3.

To improve expression of CRISPR components in mammalian cells, two genes from the SF370 locus 1 of Streptococcus pyogenes (S. pyogenes) were codon-optimized, Cas9 (SpCas9) and RNase III (SpRNase III). To facilitate nuclear localization, a nuclear localization signal (NLS) was included at the amino (N)- or carboxyl (C)-termini of both SpCas9 and SpRNase III (FIG. 63B). To facilitate visualization of protein expression, a fluorescent protein marker was also included at the N- or C-termini of both proteins (FIG. 63B). A version of SpCas9 with an NLS attached to both N- and C-terrnini (2×NLS-SpCas9) was also generated. Constructs containing NLS-fused SpCas9 and SpRNase III were transfected into 293FT human embryonic kidney (HEK) cells, and the relative positioning of the NLS to SpCas9 and SpRNase III was found to affect their nuclear localization efficiency. Whereas the C-terminal NLS was sufficient to target SpRNase III to the nucleus, attachment of a single copy of these particular NLS's to either the N- or C-terminus of SpCas9 was unable to achieve adequate nuclear localization in this system. In this example, the C-terminal NLS was that of nucleoplasmin (KRPAATKKAGQAKKKK) (SEQ ID NO: 31), and the C-terminal NLS was that of the SV40 large T-antigen (PKKKRKV) (SEQ ID NO: 30). Of the versions of SpCas9 tested, only 2×NLS-SpCas9 exhibited nuclear localization (FIG. 63B).

The tracrRNA from the CRISPR locus of S. pyogenes SF370 has two transcriptional start sites, giving rise to two transcripts of 89-nucleotides (nt) and 171 nt that are subsequently processed into identical 75 nt mature tracrRNAs. The shorter 89 nt tracrRNA was selected for expression in mammalian cells (expression constructs illustrated in FIG. 28A, with functionality as determined by results of Surveryor assay shown in FIG. 28B). Transcription start sites are marked as +1, and transcription terminator and the sequence probed by northern blot are also indicated. Expression of processed tracrRNA was also confirmed by Northern blot. FIG. 28C shows results of a Northern blot analysis of total RNA extracted from 293FT cells transfected with U6 expression constructs carrying long or short tracrRNA, as well as SpCas9 and DR-EMX1(1)-DR. Left and right panels are from 293FT cells transfected without or with SpRNase III, respectively. U6 indicate loading control blotted with a probe targeting human U6 snRNA. Transfection of the short tracrRNA expression construct led to abundant levels of the processed form of tracrRNA (˜75 bp). Very low amounts of long tracrRNA are detected on the Northern blot.

To promote precise transcriptional initiation, the RNA polymerase III-based U6 promoter was selected to drive the expression of tracrRNA (FIG. 63C). Similarly, a U6 promoter-based construct was developed to express a pre-crRNA anay consisting of a single spacer flanked by two direct repeats (DRs, also encompassed by the term “tracr-mate sequences”; FIG. 63C). The initial spacer was designed to target a 33-base-pair (bp) target site (30-bp protospacer plus a 3-bp CRISPR motif (PAM) sequence satisfying the NGG recognition motif of Cas9) in the human EMX1 locus (FIG. 63C), a key gene in the development of the cerebral cortex.

To test whether heterologous expression of the CRISPR system (SpCas9, SpRNase III, tracrRNA, and pre-crRNA) in mammalian cells can achieve targeted cleavage of mammalian chromosomes, HEK 293FT cells were transfected with combinations of CRISPR components. Since DSBs in mammalian nuclei are partially repaired by the non-homologous end joining (NHEJ) pathway, which leads to the formation of indels, the Surveyor assay was used to detect potential cleavage activity at the target EMX1 locus (FIG. 29) (see e.g. Guschin et al., 2010, Methods Mol Biol 649: 247). Co-transfection of all four CRISPR components was able to induce up to 5.0% cleavage in the protospacer (see FIG. 63D). Co-transfection of all CRISPR components minus SpRNase III also induced up to 4.7% indel in the protospacer, suggesting that there may be endogenous mammalian RNases that are capable of assisting with crRNA maturation, such as for example the related Dicer and Drosha enzymes. Removing any of the remaining three components abolished the genome cleavage activity of the CRISPR system (FIG. 63D). Sanger sequencing of amplicons containing the target locus verified the cleavage activity: in 43 sequenced clones, 5 mutated alleles (11.6%) were found. Similar experiments using a variety of guide sequences produced indel percentages as high as 29% (see FIGS. 25, 26, 67 and 28). These results define a three-component system for efficient CRISPR-mediated genome modification in mammalian cells. To optimize the cleavage efficiency, we also tested whether different isoforms of tracrRNA affected the cleavage efficiency and found that, in this example system, only the short (89-bp) transcript form was able to mediate cleavage of the human EMX1 genomic locus (FIG. 28B).

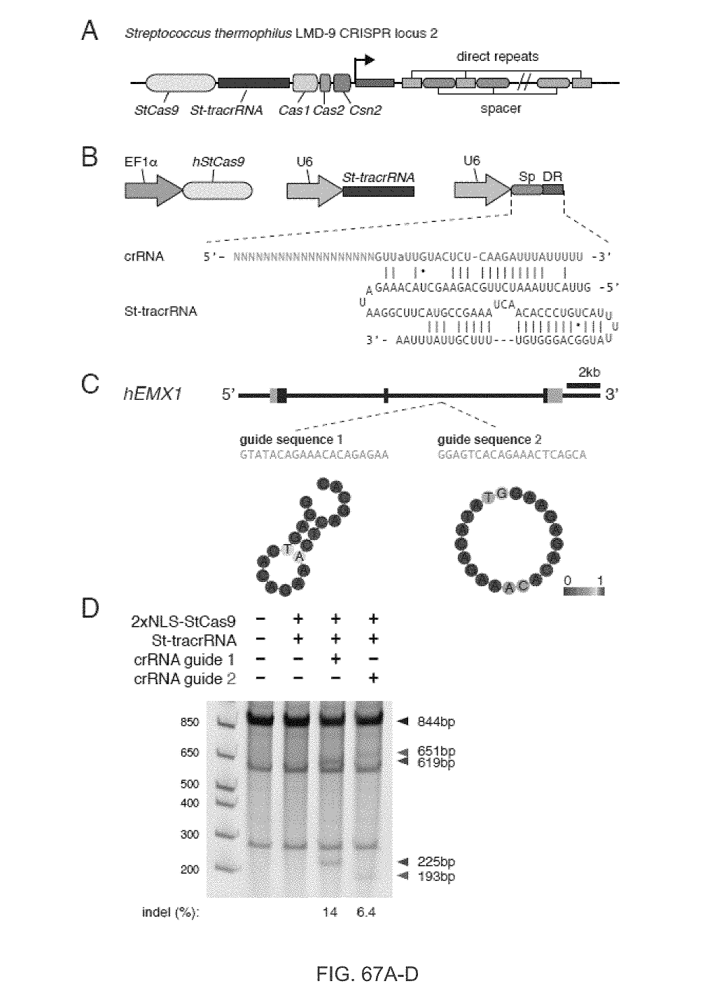

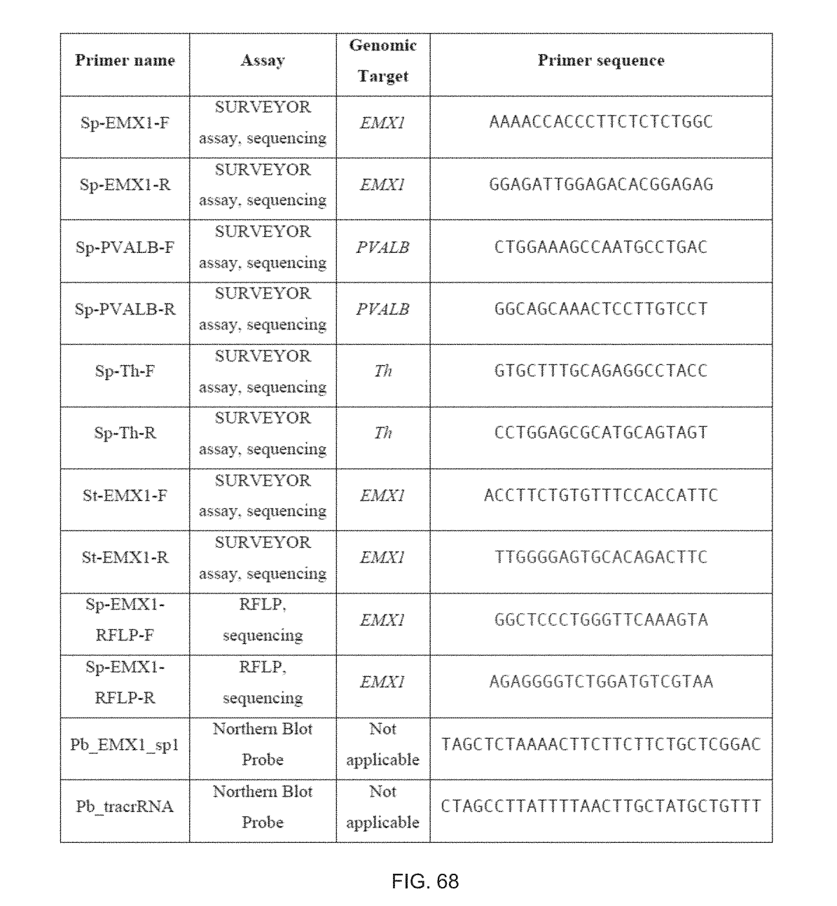

FIG. 30 provides an additional Northern blot analysis of crRNA processing in mammalian cells. FIG. 30A illustrates a schematic showing the expression vector for a single spacer flanked by two direct repeats (DR-EMX1(1)-DR). The 30 bp spacer targeting the human EMX1 locus protospacer 1 (see FIG. 67) and the direct repeat sequences are shown in the sequence beneath FIG. 30A. The line indicates the region whose reverse-complement sequence was used to generate Northern blot probes for EMX1(1) crRNA detection. FIG. 30B shows a Northern blot analysis of total RNA extracted from 293FT cells transfected with U6 expression constructs carrying DR-EMX1(1)-DR. Left and right panels are from 293FT cells transfected without or with SpRNase III respectively. DR-EMX1(1)-DR was processed into mature crRNAs only in the presence of SpCas9 and short tracrRNA and was not dependent on the presence of SpRNase III. The mature crRNA detected from transfected 293FT total RNA is ˜33 bp and is shorter than the 39-42 bp mature crRNA from S. pyogenes. These results demonstrate that a CRISPR system can be transplanted into eukaryotic cells and reprogrammed to facilitate cleavage of endogenous mammalian target polynucleotides.

FIG. 63 illustrates the bacterial CRISPR system described in this example. FIG. 63A illustrates a schematic showing the CRISPR locus 1 from Streptococcus pyogenes SF370 and a proposed mechanism of CRISPR-mediated DNA cleavage by this system. Mature crRNA processed from the direct repeat-spacer array directs Cas9 to genomic targets consisting of complimentary protospacers and a protospacer-adjacent motif (PAM). Upon target-spacer base pairing, Cas9 mediates a double-strand break in the target DNA. FIG. 63B illustrates engineering of S. pyogenes Cas9 (SpCas9) and RNase III (SpRNase III) with nuclear localization signals (NLSs) to enable import into the mammalian nucleus. FIG. 63C illustrates mammalian expression of SpCas9 and SpRNase III driven by the constitutive EF1a promoter and tracrRNA and pre-crRNA array (DR-Spacer-DR) driven by the RNA Po13 promoter U6 to promote precise transcription initiation and termination. A protospacer from the human EMX1 locus with a satisfactory PAM sequence is used as the spacer in the pre-crRNA array. FIG. 63D illustrates surveyor nuclease assay for SpCas9-mediated minor insertions and deletions. SpCas9 was expressed with and without SpRNase III, tracrRNA, and a pre-crRNA array carrying the EMX1-target spacer. FIG. 63E illustrates a schematic representation of base pairing between target locus and EMX1-targeting crRNA, as well as an example chromatogram showing a micro deletion adjacent to the SpCas9 cleavage site. FIG. 63F illustrates mutated alleles identified from sequencing analysis of 43 clonal amplicons showing a variety of micro insertions and deletions. Dashes indicate deleted bases, and non-aligned or mismatched bases indicate insertions or mutations. Scale ba=10 μm.

To further simplify the three-component system, a chimeric crRNA-tracrRNA hybrid design was adapted, where a mature crRNA (comprising a guide sequence) is fused to a partial tracrRNA via a stem-loop to mimic the natural crRNA:tracrRNA duplex (FIG. 64A). To increase co-delivery efficiency, a bicistronic expression vector was created to drive co-expression of a chimeric RNA and SpCas9 in transfected cells (FIGS. 64A and 69). In parallel, the bicistronic vectors were used to express a pre-crRNA (DR-guide sequence-DR) with SpCas9, to induce processing into crRNA with a separately expressed trcrRNA (compare FIG. 24B top and bottom). FIG. 31 provides schematic illustrations of bicistronic expression vectors for pre-crRNA array (FIG. 31A) or chimeric crRNA (represented by the short line downstream of the guide sequence insertion site and upstream of the EF1a promoter in FIG. 31B) with hSpCas9, showing location of various elements and the point of guide sequence insertion. The expanded sequence around the location of the guide sequence insertion site in FIG. 31B also shows a partial DR sequence (GTTTAGAGCTA) (SEQ ID NO: 534) and a partial tracrRNA sequence (TAGCAAGTTAAAATAAGGCTAGTCCGTTTTT) (SEQ ID NO: 535). Guide sequences can be inserted between BbsI sites using annealed oligonucleotides. Sequence design for the oligonucleotides are shown below the schematic illustrations in FIG. 31, with appropriate ligation adapters indicated. WPRE represents the Woodchuck hepatitis virus post-transcriptional regulatory element. The efficiency of chimeric RNA-mediated cleavage was tested by targeting the same EMX1 locus described above. Using both Surveyor assay and Sanger sequencing of amplicons, we confirmed that the chimeric RNA design facilitates cleavage of human EMX1 locus with approximately a 4.7% modification rate (FIG. 64B).

Generalizability of CRISPR-mediated cleavage in eukaryotic cells was tested by targeting additional genomic loci in both human and mouse cells by designing chimeric RNA targeting multiple sites in the human EMX1 and PVALB, as well as the mouse Th loci. FIG. 32 illustrates the selection of some additional targeted protospacers in human PVALB (FIG. 32A) and mouse Th (FIG. 32B) loci. Schematics of the gene loci and the location of three protospacers within the last exon of each are provided. The underlined sequences include 30 bp of protospacer sequence and 3 bp at the 3′ end corresponding to the PAM sequences. Protospacers on the sense and anti-sense strands are indicated above and below the DNA sequences, respectively. A modification rate of 6.3% and 0.75% was achieved for the human PVALB and mouse Th loci respectively, demonstrating the broad applicability of the CRISPR system in modifying different loci across multiple organisms (FIGS. 64B and 67). While, cleavage was only detected with one out of three spacers for each locus using the chimeric constructs, all target sequences were cleaved with efficiency of indel production reaching 27% when using the co-expressed pre-crRNA arrangement (FIG. 67).

FIG. 24 provides a further illustration that SpCas9 can be reprogrammed to target multiple genomic loci in mammalian cells. FIG. 24A provides a schematic of the human EMX1 locus showing the location of five protospacers, indicated by the underlined sequences. FIG. 24B provides a schematic of the pre-crRNA/trcrRNA complex showing hybridization between the direct repeat region of the pre-crRNA and tracrRNA (top), and a schematic of a chimeric RNA design comprising a 20 bp guide sequence, and tracr mate and tracr sequences consisting of partial direct repeat and tracrRNA sequences hybridized in a hairpin structure (bottom). Results of a Surveyor assay comparing the efficacy of Cas9-mediated cleavage at five protospacers in the human EMX1 locus is illustrated in FIG. 24C. Each protospacer is targeted using either processed pre-crRNA/tracrRNA complex (crRNA) or chimeric RNA (chiRNA).

Since the secondary structure of RNA can be crucial for intermolecular interactions, a structure prediction algorithm based on minimum free energy and Boltzmann-weighted structure ensemble was used to compare the putative secondary structure of all guide sequences used in our genome targeting experiment (FIG. 64B) (see e.g. Gruber et al., 2008, Nucleic Acids Research, 36: W70). Analysis revealed that in most cases, the effective guide sequence in the chimeric crRNA context were substantially free of secondary structure motifs, whereas the ineffective guide sequences were more likely to form internal secondary structures that could prevent base pairing with the target protospacer DNA. It is thus possible that variability in the spacer secondary structure might impact the efficiency of CRISPR-mediated interference when using a chimeric crRNA.

FIG. 64 illustrates example expression vectors. FIG. 64A provides a schematic of a bi-cistronic vector for driving the expression of a synthetic crRNA-tracrRNA chimera (chimeric RNA) as well as SpCas9. The chimeric guide RNA contains a 20-bp guide sequence corresponding to the protospacer in the genomic target site. FIG. 64B provides a schematic showing guide sequences targeting the human EMX1, PVALB, and mouse Th loci, as well as their predicted secondary structures. The modification efficiency at each target site is indicated below the RNA secondary structure drawing (EMX1, n=216 amplicon sequencing reads; PVALB, n=224 reads; Th, n=265 reads). The folding algorithm produced an output with each base colored according to its probability of assuming the predicted secondary structure, as indicated by a rainbow scale that is reproduced in FIG. 64B in gray scale.

To test whether spacers containing secondary structures are able to function in prokaryotic cells where CRISPRs naturally operate, transformation interference of protospacer-bearing plasmids were tested in an E. coli strain heterologously expressing the S. pyogenes SF370 CRISPR locus 1 (FIG. 70). The CRISPR locus was cloned into a low-copy E. coli expression vector and the crRNA array was replaced with a single spacer flanked by a pair of DRs (pCRISPR). E. coli strains harboring different pCRISPR plasmids were transformed with challenge plasmids containing the corresponding protospacer and PAM sequences (FIG. 70C). In the bacterial assay, all spacers facilitated efficient CRISPR interference (FIG. 64C). These results suggest that there may be additional factors affecting the efficiency of CRISPR activity in mammalian cells.

To investigate the specificity of CRISPR-mediated cleavage, the effect of single-nucleotide mutations in the guide sequence on protospacer cleavage in the mammalian genome was analyzed using a series of EMX1-targeting chimeric crRNAs with single point mutations (FIG. 25A). FIG. 25B illustrates results of a Surveyor nuclease assay comparing the cleavage efficiency of Cas9 when paired with different mutant chimeric RNAs. Single-base mismatch up to 12-bp 5′ of the PAM substantially abrogated genomic cleavage by SpCas9, whereas spacers with mutations at farther upstream positions retained activity against the original protospacer target (FIG. 25B). In addition to the PAM, SpCas9 has single-base specificity within the last 12-bp of the spacer. Furthermore, CRISPR is able to mediate genomic cleavage as efficiently as a pair of TALE nucleases (TALEN) targeting the same EMX1 protospacer. FIG. 25C provides a schematic showing the design of TALENs targeting EMX1, and FIG. 25D shows a Surveyor gel comparing the efficiency of TALEN and Cas9 (n=3).

Having established a set of components for achieving CRISPR-mediated gene editing in mammalian cells through the error-prone NHEJ mechanism, the ability of CRISPR to stimulate homologous recombination (HR), a high fidelity gene repair pathway for making precise edits in the genome, was tested. The wild type SpCas9 is able to mediate site-specific DSB, which can be repaired through both NHEJ and HR. In addition, an aspartate-to-alanine substitution (D10A) in the RuvC I catalytic domain of SpCas9 was engineered to convert the nuclease into a nickase (SpCas9n; illustrated in FIG. 26A) (see e.g. Sapranausaks et al., 2011, Cucleic Acis Research, 39: 9275; Gasiunas et al., 2012, Proc. Natl. Acad. Sci. USA, 109:E2579), such that nicked genomic DNA undergoes the high-fidelity homology-directed repair (HDR). Surveyor assay confirmed that SpCas9n does not generate indels at the EMX1 protospacer target. As illustrated in FIG. 26B, co-expression of EMX1-targeting chimeric crRNA with SpCas9 produced indels in the target site, whereas co-expression with SpCas9n did not (n=3). Moreover, sequencing of 327 amplicons did not detect any indels induced by SpCas9n. The same locus was selected to test CRISPR-mediated HR by co-transfecting HEK 293FT cells with the chimeric RNA targeting EMX1, hSpCas9 or hSpCas9n, as well as a HR template to introduce a pair of restriction sites (HindIII and NheI) near the protospacer. FIG. 26C provides a schematic illustration of the HR strategy, with relative locations of recombination points and primer annealing sequences (arrows). SpCas9 and SpCas9n indeed catalyzed integration of the HR template into the EMX1 locus. PCR amplification of the target region followed by restriction digest with HindIII revealed cleavage products corresponding to expected fragment sizes (arrows in restriction fragment length polymorphism gel analysis shown in FIG. 26D), with SpCas9 and SpCas9n mediating similar levels of HR efficiencies. We further verified HR using Sanger sequencing of genomic amplicons (FIG. 26E). These results demonstrate the utility of CRISPR for facilitating targeted gene insertion in the mammalian genome. Given the 14-bp (12-bp from the spacer and 2-bp from the PAM) target specificity of the wild type SpCas9, the availability of a nickase can significantly reduce the likelihood of off-target modifications, since single strand breaks are not substrates for the error-prone NHEJ pathway.

Expression constructs mimicking the natural architecture of CRISPR loci with arrayed spacers (FIG. 63A) were constructed to test the possibility of multiplexed sequence targeting. Using a single CRISPR array encoding a pair of EMX1- and PVALB-targeting spacers, efficient cleavage at both loci was detected (FIG. 26F, showing both a schematic design of the crRNA anay and a Surveyor blot showing efficient mediation of cleavage). Targeted deletion of larger genomic regions through concurrent DSBs using spacers against two targets within EMX1 spaced by 119 bp was also tested, and a 1.6% deletion efficacy (3 out of 182 amplicons; FIG. 26G) was detected. This demonstrates that the CRISPR system can mediate multiplexed editing within a single genome.

### Example 15: CRISPR System Modifications and Alternatives

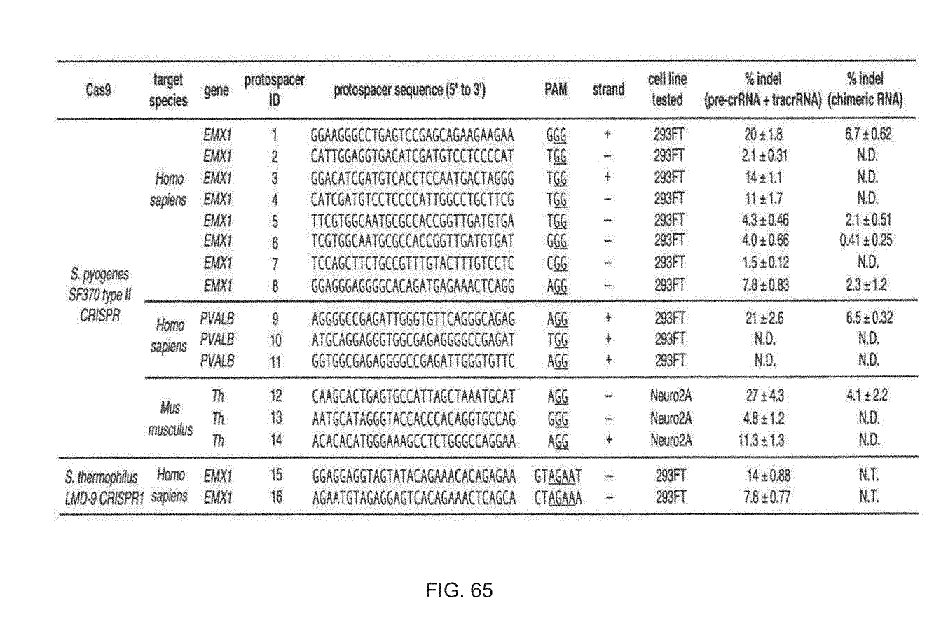

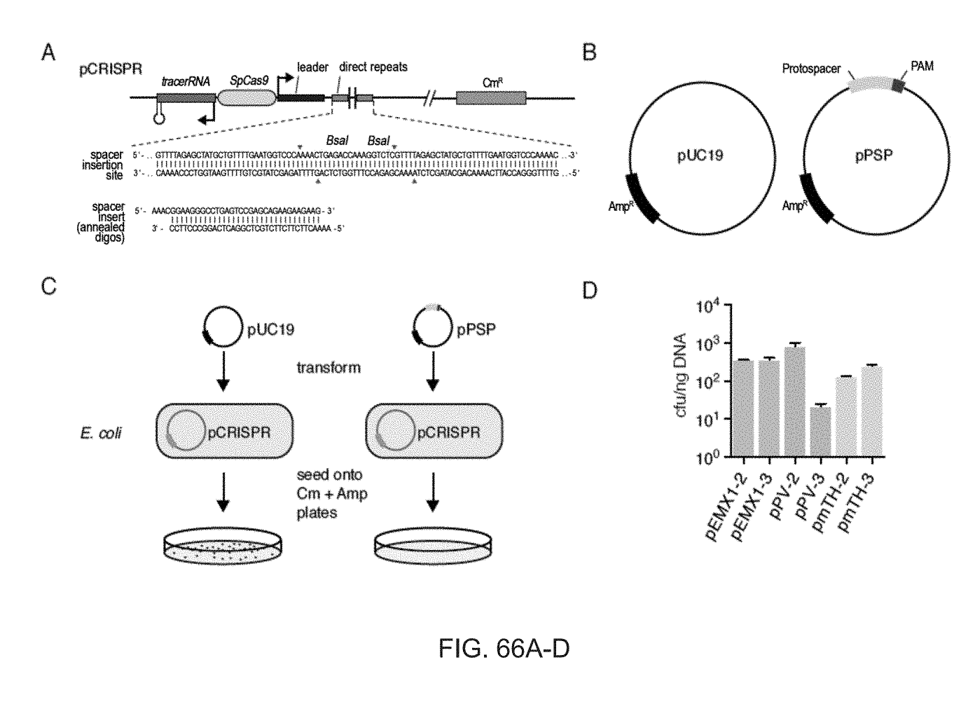

The ability to use RNA to program sequence-specific DNA cleavage defines a new class of genome engineering tools for a variety of research and industrial applications. Several aspects of the CRISPR system can be further improved to increase the efficiency and versatility of CRISPR targeting. Optimal Cas9 activity may depend on the availability of free Mg²⁺ at levels higher than that present in the mammalian nucleus (see e.g. Jinek et al., 2012, Science, 337:816), and the preference for an NGG motif immediately downstream of the protospacer restricts the ability to target on average every 12-bp in the human genome (FIG. 33, evaluating both plus and minus strands of human chromosomal sequences). Some of these constraints can be overcome by exploring the diversity of CRISPR loci across the microbial metagenome (see e.g. Makarova et al., 2011, Nat Rev Microbiol, 9:467). Other CRISPR loci may be transplanted into the mammalian cellular milieu by a process similar to that described in Example 1. For example, FIG. 67 illustrates adaptation of the Type II CRISPR system from CRISPR locus 2 of Streptococcus thermophilus LMD-9 for heterologous expression in mammalian cells to achieve CRISPR-mediated genome editing. FIG. 67A provides a Schematic illustration of the CRISPR locus 2 from S. thermophilus LMD-9. FIG. 67B illustrates the design of an expression system for the S. thermophilus CRISPR system. Human codon-optimized hStCas9 is expressed using a constitutive EF1ca promoter. Mature versions of tracrRNA and crRNA are expressed using the U6 promoter to promote precise transcription initiation. Sequences from the mature crRNA and tracrRNA are illustrated. A single base indicated by the lower case “a” in the crRNA sequence is used to remove the polyU sequence, which serves as a RNA polII transcriptional terminator. FIG. 67C provides a schematic showing guide sequences targeting the human EMX1 locus as well as their predicted secondary structures. The modification efficiency at each target site is indicated below the RNA secondary structures. The algorithm generating the structures colors each base according to its probability of assuming the predicted secondary structure, which is indicated by a rainbow scale reproduced in FIG. 67C in gray scale. FIG. 67D shows the results of hStCas9-mediated cleavage in the target locus using the Surveyor assay. RNA guide spacers 1 and 2 induced 14% and 6.4%, respectively. Statistical analysis of cleavage activity across biological replica at these two protospacer sites is also provided in FIG. 65. FIG. 34C provides a schematic of additional protospacer and corresponding PAM sequence targets of the S. thermophilus CRISPR system in the human EMX1 locus. Two protospacer sequences are highlighted and their corresponding PAM sequences satisfying NNAGAAW motif are indicated by underlining 3′ with respect to the corresponding highlighted sequence. Both protospacers target the anti-sense strand.

### Example 16: Sample Target Sequence Selection Algorithm

A software program is designed to identify candidate CRISPR target sequences on both strands of an input DNA sequence based on desired guide sequence length and a CRISPR motif sequence (PAM) for a specified CRISPR enzyme. For example, target sites for Cas9 from S. pyogenes, with PAM sequences NGG, may be identified by searching for 5′-Nx-NGG-3′ both on the input sequence and on the reverse-complement of the input. Likewise, target sites for Cas9 of S. thermophilus CRISPR1, with PAM sequence NNAGAAW, may be identified by searching for 5′-Nₓ-NNAGAAW-3′ (SEQ ID NO: 536) both on the input sequence and on the reverse-complement of the input. Likewise, target sites for Cas9 of S. thermophilus CRISPR3, with PAM sequence NGGNG, may be identified by searching for 5′-Nₓ-NGGNG-3′ both on the input sequence and on the reverse-complement of the input. The value “x” in Nₓ may be fixed by the program or specified by the user, such as 20.

Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a ‘seed’ sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed:PAM sequence in the relevant genome. The user may be allowed to choose the length of the seed sequence. The user may also be allowed to specify the number of occurrences of the seed:PAM sequence in a genome for purposes of passing the filter. The default is to screen for unique sequences. Filtration level is altered by changing both the length of the seed sequence andthe number of occurrences of the sequence in the genome. The program may in addition or alternatively provide the sequence of a guide sequence complementary to the reported target sequence(s) by providing the reverse complement of the identified target sequence(s).

This target sequence identifier tool is applicable for identifying target in any genome, such as human, mouse, rate, and C. elegans.

Sequences (SEQ ID NOS 74-77, 537-543, 81, 599 and 83, respectively, in order of appearance) described in the above examples are as follows:

U6-short tracrRNA (Streptococcus pyogenes
SF370):
GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACCG
GAACCATTCAAAACAGCATAGCAAGTTAAAATAAGGCTAGTCCGTTATCA
ACTTGAAAAAGTGGCACCGAGTCGGTGCTTTTTTT
U6-long tracrRNA (Streptococcus pyogenes
SF370):
GAGGGCCCATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACCG
GTAGTATTAAGTATTGTTTTATGGCTGATAAATTTCTTTGAATTTCTCCT
TGATTATTTGTTATAAAAGTTATAAAATAATCTTGTTGGAACCATTCAAA
ACAGCATAGCAAGTTAAAATAAGGCTAGTCCGTTATCAACTTGAAAAAGT
GGCACCGAGTCGGTGCTTTTTTT
U6-DR-BbsI backbone-DR (Streptococcus
pyogenes SF370):
GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACCG
GGTTTTAGAGCTATGCTGTTTTGAATGGTCCCAAAACGGGTCTTCGAGAA
GACGTTTTAGAGCTATGCTGTTTTGAATGGTCCCAAAAC
U6-chimeric RNA-BbsI backbone (Streptococcus
pyogenes SF370)
GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACCG
GGTCTTCGAGAAGACCTGTTTTAGAGCTAGAAATAGCAAGTTAAAATAAG
GCTAGTCCG
NLS-SpCas9-EGFP:
MDYKDHDGDYKDHDIDYKDDDDKMAPKKKRKVGIHGVPAADKKYSIGLDI
GTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTE
ITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDAAAVSKGEELFTGVVPILVELDGDVNGHKFSVSGEGEGDATYG
KLTLKFICTTGKLPVPWPTLVTTLTYGVQCFSRYPDHMKQHDFFKSAMPE
GYVQERTIFFKDDGNYKTRAEVKFEGDTLVNRIELKGIDFKEDGNILGHK
LEYNYNSHNVYIMADKQKNGIKVNFKIRHNIEDGSVQLADHYQQNTPIGD
GPVLLPDNHYLSTQSALSKDPNEKRDHMVLLEFVTAAGITLGMDELYK
SpCas9-EGFP-NLS:
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGDAAAVSKGEELFTGVVPILVELDGDVNGHKFSV
SGEGEGDATYGKLTLKFICTTGKLPVPWPTLVTTLTYGVQCFSRYPDHMK
QHDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQLA
DHYQQNTPIGDGPVLLPDNHYLSTQSALSKDPNEKRDHMVLLEFVTAAGI
TLGMDELYKKRPAATKKAGQAKKKK
NLS-SpCas9-EGFP-NLS:
MDYKDHDGDYKDHDIDYKDDDDKMAPKKKRKVGIHGVPAADKKYSIGLDI
GTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTE
ITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDAAAVSKGEELFTGVVPILVELDGDVNGHKFSVSGEGEGDATYG
KLTLKFICTTGKLPVPWPTLVTTLTYGVQCFSRYPDHMKQHDFFKSAMPE
GYVQERTIFFKDDGNYKTRAEVKFEGDTLVNRIELKGIDFKEDGNILGHK
LEYNYNSHNVYIMADKQKNGIKVNFKIRHNIEDGSVQLADHYQQNTPIGD
GPVLLPDNHYLSTQSALSKDPNEKRDHMVLLEFVTAAGITLGMDELYKKR
PAATKKAGQAKKKK
NLS-SpCas9-NLS:
MDYKDHDGDYKDHDIDYKDDDDKMAPKKKRKVGIHGVPAADKKYSIGLDI
GTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTE
ITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDKRPAATKKAGQAKKKK
NLS-mCherry-SpRNase3:
MFLFLSLTSFLSSSRTLVSKGEEDNMAIIKEFMRFKVHMEGSVNGHEFEI
EGEGEGRPYEGTQTAKLKVTKGGPLPFAWDILSPQFMYGSKAYVKHPADI
PDYLKLSFPEGFKWERVMNFEDGGVVTVTQDSSLQDGEFIYKVKLRGTNF
PSDGPVMQKKTMGWEASSERMYPEDGALKGEIKQRLKLKDGGHYDAEVKT
TYKAKKPVQLPGAYNVNIKLDITSHNEDYTIVEQYERAEGRHSTGGMDEL
YKGSKQLEELLSTSFDIQFNDLTLLETAFTHTSYANEHRLLNVSHNERLE
FLGDAVLQLIISEYLFAKYPKKTEGDMSKLRSMIVREESLAGFSRFCSFD
AYIKLGKGEEKSGGRRRDTILGDLFEAFLGALLLDKGIDAVRRFLKQVMI
PQVEKGNFERVKDYKTCLQEFLQTKGDVAIDYQVISEKGPAHAKQFEVSI
VVNGAVLSKGLGKSKKLAEQDAAKNALAQLSEV
SpRNase3-mCherry-NLS:
MKQLEELLSTSFDIQFNDLTLLETAFTHTSYANEHRLLNVSHNERLEFLG
DAVLQLIISEYLFAKYPKKTEGDMSKLRSMIVREESLAGFSRFCSFDAYI
KLGKGEEKSGGRRRDTILGDLFEAFLGALLLDKGIDAVRRFLKQVMIPQV
EKGNFERVKDYKTCLQEFLQTKGDVAIDYQVISEKGPAHAKQFEVSIVVN
GAVLSKGLGKSKKLAEQDAAKNALAQLSEVGSVSKGEEDNMAIIKEFMRF
KVHMEGSVNGHEFEIEGEGEGRPYEGTQTAKLKVTKGGPLPFAWDILSPQ
FMYGSKAYVKHPADIPDYLKLSFPEGFKWERVMNFEDGGVVTVTQDSSLQ
DGEFIYKVKLRGTNFPSDGPVMQKKTMGWEASSERMYPEDGALKGEIKQR
LKLKDGGHYDAEVKTTYKAKKPVQLPGAYNVNIKLDITSHNEDYTIVEQY
ERAEGRHSTGGMDELYKKRPAATKKAGQAKKKK
NLS-SpCas9n-NLS (the D10A nickase mutation
is lowercase):
MDYKDHDGDYKDHDIDYKDDDDKMAPKKKRKVGIHGVPAADKKYSIGLaI
GTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTE
ITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDKRPAATKKAGQAKKKK
hEMX1-HR Template-HindII-NheI:
GAATGCTGCCCTCAGACCCGCTTCCTCCCTGTCCTTGTCTGTCCAAGGAG
AATGAGGTCTCACTGGTGGATTTCGGACTACCCTGAGGAGCTGGCACCTG
AGGGACAAGGCCCCCCACCTGCCCAGCTCCAGCCTCTGATGAGGGGTGGG
AGAGAGCTACATGAGGTTGCTAAGAAAGCCTCCCCTGAAGGAGACCACAC
AGTGTGTGAGGTTGGAGTCTCTAGCAGCGGGTTCTGTGCCCCCAGGGATA
GTCTGGCTGTCCAGGCACTGCTCTTGATATAAACACCACCTCCTAGTTAT
GAAACCATGCCCATTCTGCCTCTCTGTATGGAAAAGAGCATGGGGCTGGC
CCGTGGGGTGGTGTCCACTTTAGGCCCTGTGGGAGATCATGGGAACCCAC
GCAGTGGGTCATAGGCTCTCTCATTTACTACTCACATCCACTCTGTGAAG
AAGCGATTATGATCTCTCCTCTAGAAACTCGTAGAGTCCCATGTCTGCCG
GCTTCCAGAGCCTGCACTCCTCCACCTTGGCTTGGCTTTGCTGGGGCTAG
AGGAGCTAGGATGCACAGCAGCTCTGTGACCCTTTGTTTGAGAGGAACAG
GAAAACCACCCTTCTCTCTGGCCCACTGTGTCCTCTTCCTGCCCTGCCAT
CCCCTTCTGTGAATGTTAGACCCATGGGAGCAGCTGGTCAGAGGGGACCC
CGGCCTGGGGCCCCTAACCCTATGTAGCCTCAGTCTTCCCATCAGGCTCT
CAGCTCAGCCTGAGTGTTGAGGCCCCAGTGGCTGCTCTGGGGGCCTCCTG
AGTTTCTCATCTGTGCCCCTCCCTCCCTGGCCCAGGTGAAGGTGTGGTTC
CAGAACCGGAGGACAAAGTACAAACGGCAGAAGCTGGAGGAGGAAGGGCC
TGAGTCCGAGCAGAAGAAGAAGGGCTCCCATCACATCAACCGGTGGCGCA
TTGCCACGAAGCAGGCCAATGGGGAGGACATCGATGTCACCTCCAATGAC
aagcttgctagcGGTGGGCAACCACAAACCCACGAGGGCAGAGTGCTGCT
TGCTGCTGGCCAGGCCCCTGCGTGGGCCCAAGCTGGACTCTGGCCACTCC
CTGGCCAGGCTTTGGGGAGGCCTGGAGTCATGGCCCCACAGGGCTTGAAG
CCCGGGGCCGCCATTGACAGAGGGACAAGCAATGGGCTGGCTGAGGCCTG
GGACCACTTGGCCTTCTCCTCGGAGAGCCTGCCTGCCTGGGCGGGCCCGC
CCGCCACCGCAGCCTCCCAGCTGCTCTCCGTGTCTCCAATCTCCCTTTTG
TTTTGATGCATTTCTGTTTTAATTTATTTTCCAGGCACCACTGTAGTTTA
GTGATCCCCAGTGTCCCCCTTCCCTATGGGAATAATAAAAGTCTCTCTCT
TAATGACACGGGCATCCAGCTCCAGCCCCAGAGCCTGGGGTGGTAGATTC
CGGCTCTGAGGGCCAGTGGGGGCTGGTAGAGCAAACGCGTTCAGGGCCTG
GGAGCCTGGGGTGGGGTACTGGTGGAGGGGGTCAAGGGTAATTCATTAAC
TCCTCTCTTTTGTTGGGGGACCCTGGTCTCTACCTCCAGCTCCACAGCAG
GAGAAACAGGCTAGACATAGGGAAGGGCCATCCTGTATCTTGAGGGAGGA
CAGGCCCAGGTCTTTCTTAACGTATTGAGAGGTGGGAATCAGGCCCAGGT
AGTTCAATGGGAGAGGGAGAGTGCTTCCCTCTGCCTAGAGACTCTGGTGG
CTTCTCCAGTTGAGGAGAAACCAGAGGAAAGGGGAGGATTGGGGTCTGGG
GGAGGGAACACCATTCACAAAGGCTGACGGTTCCAGTCCGAAGTCGTGGG
CCCACCAGGATGCTCACCTGTCCTTGGAGAACCGCTGGGCAGGTTGAGAC
TGCAGAGACAGGGCTTAAGGCTGAGCCTGCAACCAGTCCCCAGTGACTCA
GGGCCTCCTCAGCCCAAGAAAGAGCAACGTGCCAGGGCCCGCTGAGCTCT
TGTGTTCACCTG
NLS-StCsn1-NLS:
MKRPAATKKAGQAKKKKSDLVLGLDIGIGSVGVGILNKVTGEIIHKNSRI
FPAAQAENNLVRRTNRQGRRLARRKKHRRVRLNRLFEESGLITDFTKISI
NLNPYQLRVKGLTDELSNEELFIALKNMVKHRGISYLDDASDDGNSSVGD
YAQIVKENSKQLETKTPGQIQLERYQTYGQLRGDFTVEKDGKKHRLINVF
PTSAYRSEALRILQTQQEFNPQITDEFINRYLEILTGKRKYYHGPGNEKS
RTDYGRYRTSGETLDNIFGILIGKCTFYPDEFRAAKASYTAQEFNLLNDL
NNLTVPTETKKLSKEQKNQIINYVKNEKAMGPAKLFKYIAKLLSCDVADI
KGYRIDKSGKAEIHTFEAYRKMKTLETLDIEQMDRETLDKLAYVLTLNTE
REGIQEALEHEFADGSFSQKQVDELVQFRKANSSIFGKGWHNFSVKLMME
LIPELYETSEEQMTILTRLGKQKTTSSSNKTKYIDEKLLTEEIYNPVVAK
SVRQAIKIVNAAIKEYGDFDNIVIEMARETNEDDEKKAIQKIQKANKDEK
DAAMLKAANQYNGKAELPHSVFHGHKQLATKIRLWHQQGERCLYTGKTIS
IHDLINNSNQFEVDHILPLSITFDDSLANKVLVYATANQEKGQRTPYQAL
DSMDDAWSFRELKAFVRESKTLSNKKKEYLLTEEDISKFDVRKKFIERNL
VDTRYASRVVLNALQEHFRAHKIDTKVSVVRGQFTSQLRRHWGIEKTRDT
YHHHAVDALIIAASSQLNLWKKQKNTLVSYSEDQLLDIETGELISDDEYK
ESVFKAPYQHFVDTLKSKEFEDSILFSYQVDSKFNRKISDATIYATRQAK
VGKDKADETYVLGKIKDIYTQDGYDAFMKIYKKDKSKFLMYRHDPQTFEK
VIEPILENYPNKQINEKGKEVPCNPFLKYKEEHGYIRKYSKKGNGPEIKS
LKYYDSKLGNHIDITPKDSNNKVVLQSVSPWRADVYFNKTTGKYEILGLK
YADLQFEKGTGTYKISQEKYNDIKKKEGVDSDSEFKFTLYKNDLLLVKDT
ETKEQQLFRFLSRTMPKQKHYVELKPYDKQKFEGGEALIKVLGNVANSGQ
CKKGLGKSNISIYKVRTDVLGNQHIIKNEGDKPKLDFKRPAATKKAGQAK
KKK
U6-St_tracrRNA(7-97):
GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACCG
TTACTTAAATCTTGCAGAAGCTACAAAGATAAGGCTTCATGCCGAAATCA
ACACCCTGTCATTTTATGGCAGGGTGTTTTCGTTATTTAA

The invention is further described by the following numbered paragraphs:

1. An inducible method of altering expression of a genomic locus of interest in a cell comprising:

(a) contacting the genomic locus with a non-naturally occurring or engineered composition comprising a deoxyribonucleic acid (DNA) binding polypeptide comprising:
(i) a DNA binding domain comprising at least five or more Transcription activator-like effector (TALE) monomers and at least one or more half-monomers specifically ordered to target the genomic locus of interest or
at least one or more effector domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers specifically ordered to target the genomic locus of interest or
at least one or more effector domains
linked to the interacting partner, wherein the energy sensitive protein or fragment thereof binds to the interacting partner upon induction by the energy source;

(b) applying the energy source; and

(c) determining that the expression of the genomic locus is altered.

2. The method according to paragraph 1, wherein the at least one or more effector domains is selected from the group consisting of: transposase domain, integrase domain, recombinase domain, resolvase domain, invertase domain, protease domain, DNA methyltransferase domain, DNA demethylase domain, histone acetylase domain, histone deacetylases domain, nuclease domain, repressor domain, activator domain, nuclear-localization signal domains, transcription-protein recruiting domain, cellular uptake activity associated domain, nucleic acid binding domain and antibody presentation domain.

3. The method according to paragraph 2, wherein the at least one or more effector domains is a nuclease domain or a recombinase domain.

4. The method according to paragraph 3, wherein the nuclease domain is a non-specific FokI endonuclease catalytic domain.

5. The method according to any one of paragraphs 1-4, wherein the energy sensitive protein is Cryptochrome-2 (CRY2).

6. The method according to any one of paragraphs 1-5, wherein the interacting partner is Cryptochrome-interacting basic helix-loop-helix (CIB1).

7. The method according to any of paragraphs 1-6, wherein the energy source is selected from the group consisting of: electromagnetic radiation, sound energy or thermal energy.

8. The method according to paragraph 7, wherein the electromagnetic radiation is a component of visible light.

9. The method according to paragraph 8, wherein the component of visible light has a wavelength in the range of 450 nm-500 nm.

10. The method according to paragraph 8, wherein the component of visible light is blue light.

11. The method according to paragraph 1, wherein the applying the energy source comprises stimulation with blue light at an intensity of at least 6.2 mW/cm².

12. The method according to any one of paragraphs 1-11, wherein the DNA binding domain comprises (X₁₋₁₁-X₁₂X₁₃-X_{14-33 or 34 or 35})_{z},

wherein X₁₋₁₁ is a chain of 11 contiguous amino acids,

wherein X₁₂X₁₃ is a repeat variable diresidue (RVD),

wherein X_{14-33 or 34 or 35} is a chain of 21, 22 or 23 contiguous amino acids,

wherein z is at least 5 to 40, and

wherein at least one RVD is selected from the group consisting of NI, HD, NG, NN, KN, RN, NH, NQ, SS, SN, NK, KH, RH, HH, HI, KI, RI, SI, KG, HG, RG, SD, ND, KD, RD, YG, HN, NV, NS, HA, S*, N*, KA, H*, RA, NA, and NC, wherein (*) means that the amino acid at X₁₃ is absent.

13. The method according to paragraph 12, wherein z is at least 10 to 26.

14. The method according to paragraph 12, wherein

at least one of X₁₋₁₁ is a sequence of 12 contiguous amino acids set forth as amino acids 1-11 in a sequence (X₁₋₁₁-X₁₄₋₃₄ or X₁₋₁₁-X₁₄₋₃₅) of FIG. 9 or

at least one of X₁₄₋₃₄ or X₁₄₋₃₅ is a sequence of 21 or 22 contiguous amino acids set forth as amino acids 12-32 or 12-33 in a sequence (X₁₋₁₁-X₁₄₋₃₄ or X₁₋₁₁-X₁₄₋₃₅) of FIG. 9.

15. The method according to paragraph 12, wherein the at least one RVD is selected from the group consisting of (a) HH, KH, NH, NK, NQ, RH, RN, SS for recognition of guanine (G); (b) SI for recognition of adenine (A); (c) HG, KG, RG for recognition of thymine (T); (d) RD, SD for recognition of cytosine (C); (e) NV for recognition of A or G; and (f) H*, HA, KA, N*, NA, NC, NS, RA, S* for recognition of A or T or G or C, wherein (*) means that the amino acid at X₁₃ is absent.

16. The method according to paragraph 15, wherein
the RVD for the recognition of G is RN, NH, RH or KH; or
the RVD for the recognition of A is SI; or
the RVD for the recognition of T is KG or RG; and
the RVD for the recognition of C is SD or RD.

17. The method according to paragraph 12, wherein at least one of the following is present
[LTLD] (SEQ ID NO: 1) or [LTLA] (SEQ ID NO: 2) or [LTQV] (SEQ ID NO: 3) at X₁₋₄, or
[EQHG] (SEQ ID NO: 4) or [RDHG] (SEQ ID NO: 5) at positions X₃₀₋₃₃ or X₃₁₋₃₄ or X₃₂₋₃₅.

18. The method according to any one of paragraphs 1-17, wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

19. The method according to any one of paragraphs 1-18, wherein the genomic locus of interest is associated with a gene that encodes for a differentiation factor, a transcription factor, a neurotransmitter transporter, a neurotransmitter synthase, a synaptic protein, a plasticity protein, a presynaptic active zone protein, a post synaptic density protein, a neurotransmitter receptor, an epigenetic modifier, a neural fate specification factor, an axon guidance molecule, an ion channel, a CpG binding protein, a ubiquitination protein, a hormone, a homeobox protein, a growth factor, an oncogenes or a proto-oncogene.

20. An inducible method of repressing expression of a genomic locus of interest in a cell comprising:

(a) contacting the genomic locus with a non-naturally occurring or engineered composition comprising a DNA binding polypeptide comprising:
(i) a DNA binding domain comprising at least five or more Transcription activator-like effector (TALE) monomers and at least one or more half-monomers specifically ordered to target the genomic locus of interest or
at least one or more effector domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers specifically ordered to target the genomic locus of interest or
at least one or more effector domains
linked to the interacting partner, wherein the energy sensitive protein or fragment thereof binds to the interacting partner upon induction by the energy source;

(b) applying the energy source; and

(c) determining that the expression of the genomic locus is repressed.

21. The method according to paragraph 20, wherein the polypeptide includes at least one SID repressor domain.

22. The method according to paragraph 21, wherein the polypeptide includes at least four SID repressor domains.

23. The method according to paragraph 21, wherein the polypeptide includes a SID4X repressor domain.

24. The method according to paragraph 20, wherein the polypeptide includes a KRAB repressor domain.

25. The method according to any one of paragraphs 20-24, wherein the energy sensitive protein is Cryptochrome-2 (CRY2).

26. The method according to any one of paragraphs 20-25, wherein the interacting partner is Cryptochrome-interacting basic helix-loop-helix (CIB1).

27. The method according to any one of paragraphs 20-26, wherein the energy source is selected from the group consisting of: electromagnetic radiation, sound energy or thermal energy.

28. The method according to paragraph 20, wherein the electromagnetic radiation is a component of visible light.

29. The method according to paragraph 28, wherein the component of visible light has a wavelength in the range of 450 nm-500 nm.

30. The method according to paragraph 28, wherein the component of visible light is blue light.

31. The method according to paragraph 20, wherein the applying the energy source comprises stimulation with blue light at an intensity of at least 6.2 mW/cm².

32. The method according to paragraph 20-31, wherein the DNA binding domain comprises (X₁₋₁₁-X₁₂X₁₃-X₁₄₋₃₃ or 34 or 35)_{z},

wherein X₁₋₁₁ is a chain of 11 contiguous amino acids,

wherein X₁₂X₁₃ is a repeat variable diresidue (RVD),

wherein X_{14-33 or 34 or 35} is a chain of 21, 22 or 23 contiguous amino acids,

wherein z is at least 5 to 40, and

wherein at least one RVD is selected from the group consisting of NI, HD, NG, NN, KN, RN, NH, NQ, SS, SN, NK, KH, RH, HH, HI, KI, RI, SI, KG, HG, RG, SD, ND, KD, RD, YG, HN, NV, NS, HA, S*, N*, KA, H*, RA, NA, and NC, wherein (*) means that the amino acid at X₁₃ is absent.

33. The method according to paragraph 32, wherein z is at least 10 to 26.

34. The method according to paragraph 32, wherein

at least one of X₁₋₁₁ is a sequence of 11 contiguous amino acids set forth as amino acids 1-11 in a sequence (X₁₋₁₁-X₁₄₋₃₄ or X₁₋₁₁-X₁₄₋₃₅) of FIG. 9 or

at least one of X₁₄₋₃₄ or X₁₄₋₃₅ is a sequence of 21 or 22 contiguous amino acids set forth as amino acids 12-32 or 12-33 in a sequence (X₁₋₁₁-X₁₄₋₃₄ or X₁₋₁₁-X₁₄₋₃₅) of FIG. 9.

35. The method according to any one of paragraphs 20-34, wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

36. The method according to any one of paragraphs 20-35, wherein the genomic locus of interest is the genomic locus associated with a gene that encodes for a differentiation factor or a component of an ion channel.

37. The method according to paragraph 36, wherein the differentiation factor is SRY-box-2 (SOX2) and is encoded by the gene SOX2.

38. The method according to paragraph 36, wherein the differentiation factor is p11 and is encoded by the gene p11.

39. The method according to paragraph 36, wherein the component of the ion channel is CACNA1C and is encoded by the gene CACNA1C.

40. An inducible method of activating expression of a genomic locus of interest in a cell comprising:

(a) contacting the genomic locus with a non-naturally occurring or engineered composition comprising a DNA binding polypeptide comprising:
(i) a DNA binding domain comprising at least five or more TALE monomers and at least one or more half-monomers specifically ordered to target the genomic locus of interest or
at least one or more activator domains
linked to an energy sensitive protein or fragment thereof, wherein the energy sensitive protein or fragment thereof undergoes a conformational change upon induction by an energy source allowing it to bind an interacting partner, and
(ii) a DNA binding domain comprising at least one or more TALE monomers or half-monomers specifically ordered to target the genomic locus of interest or
at least one or more activator domains
linked to the interacting partner, wherein the energy sensitive protein or fragment thereof binds to the interacting partner upon induction by the energy source;

(b) applying the energy source; and

(c) determining that the expression of the genomic locus is activated.

41. The method according to paragraph 40, wherein the polypeptide includes at least one VP16 or VP64 activator domain.

42. The method according to paragraph 40, wherein the polypeptide includes at least one p65 activator domain.

43. The method according to any one of paragraphs 40-42, wherein the energy sensitive protein is CRY2.

44. The method according to any one of paragraph 40-43, wherein the interacting partner is CIB1.

45. The method according to paragraph 40, wherein the energy source is selected from the group consisting of: electromagnetic radiation, sound energy or thermal energy.

46. The method according to paragraph 45, wherein the electromagnetic radiation is a component of visible light.

47. The method according to paragraph 46, wherein the component of visible light has a wavelength in the range of 450 nm-500 nm.

48. The method according to paragraph 46, wherein the component of visible light is blue light.

49. The method according to paragraph 40, wherein the applying the energy source comprises stimulation with blue light at an intensity of at least 6.2 mW/cm².

50. The method according to any one of paragraphs 40-49, wherein the DNA binding domain comprises (X₁₁₋₁-X₁₂X₁₃-X_{14-33 or 34 or 35})_{z},

wherein X₁₋₁₁ is a chain of 11 contiguous amino acids,

wherein X₁₂X₁₃ is a repeat variable diresidue (RVD),

wherein X_{14-33 or 34 or 35} is a chain of 21, 22 or 23 contiguous amino acids,

wherein z is at least 5 to 40, and

wherein at least one RVD is selected from the group consisting of NI, HD, NG, NN, KN, RN, NH, NQ, SS, SN, NK, KH, RH, HH, HI, KI, RI, SI, KG, HG, RG, SD, ND, KD, RD, YG, HN, NV, NS, HA, S*, N*, KA, H*, RA, NA, and NC, wherein (*) means that the amino acid at X₁₃ is absent.

51. The method according to paragraph 50, wherein z is at least 10 to 26.

52. The method according to paragraph 50, wherein

at least one of X₁₋₁₁ is a sequence of 11 contiguous amino acids set forth as amino acids 1-11 in a sequence (X₁₋₁₁-X₁₄₋₃₄ or X₁₋₁₁-X₁₄₋₃₅) of FIG. 9 or

at least one of X₁₄₋₃₄ or X₁₄₋₃₅ is a sequence of 21 or 22 contiguous amino acids set forth as amino acids 12-32 or 12-33 in a sequence (X₁₋₁₁-X₁₄₋₃₄ or X₁₋₁₁-X₁₄₋₃₅) of FIG. 9.

53. The method according to any one of paragraphs 40-52, wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

54. The method according to any one of paragraphs 40-53, wherein the genomic locus of interest is the genomic locus associated with a gene that encodes for a differentiation factor, an epigenetic modulator or a component of an ion channel.

55. The method according to paragraph 54, wherein the differentiation factor is Neurogenin-2 and is encoded by the gene NEUROG2.

56. The method according to paragraph 54, wherein the differentiation factor is Kreuppel-like factor 4 and is encoded by the gene KLF-4.

57. The method according to paragraph 54, wherein the epigenetic modulator is Tet methylcytosine dioxygenase 1 and is encoded by the gene tet-1.

58. The method according to paragraph 54, wherein the component of the ion channel is CACNA1C and is encoded by the gene CACNA1C.

59. A non-naturally occurring or engineered composition for inducibly altering expression of a genomic locus in a cell wherein the composition comprises a DNA binding polypeptide comprising:
(i) a DNA binding domain comprising at least one or more TALE monomers or half-monomers or
at least one or more effector domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers or
at least one or more effector domains
linked to the interacting partner, wherein the energy sensitive protein or fragment thereof binds to the interacting partner upon induction by the energy source;

wherein the polypeptide is encoded by and translated from a codon optimized nucleic acid molecule so that the polypeptide preferentially binds to DNA of the genomic locus, and

wherein the polypeptide alters the expression of the genomic locus upon application of the energy source.

60. The composition according to paragraph 59, wherein the at least one or more effector domains is selected from the group consisting of: transposase domain, integrase domain, recombinase domain, resolvase domain, invertase domain, protease domain, DNA methyltransferase domain, DNA demethylase domain, histone acetylase domain, histone deacetylases domain, nuclease domain, repressor domain, activator domain, nuclear-localization signal domains, transcription-protein recruiting domain, cellular uptake activity associated domain, nucleic acid binding domain and antibody presentation domain.

61. The composition according to paragraph 59, wherein the at least one or more effector domains is a nuclease domain or a recombinase domain.

62. The composition according to paragraph 61, wherein the nuclease domain is a non-specific FokI endonuclease catalytic domain.

63. The method according to any one of paragraphs 59-62, wherein the energy sensitive protein is Cryptochrome-2 (CRY2).

64. The composition according to any one of paragraphs 59-63, wherein the interacting partner is Cryptochrome-interacting basic helix-loop-helix (CIB1).

65. The composition according to any one of paragraphs 59-64, wherein the energy source is selected from the group consisting of: electromagnetic radiation, sound energy or thermal energy.

66. The composition according to paragraph 65, wherein the electromagnetic radiation is a component of visible light.

67. The composition according to paragraph 66, wherein the component of visible light has a wavelength in the range of 450 nm-500 nm.

68. The composition according to paragraph 66, wherein the component of visible light is blue light.

69. The composition according to paragraph 59, wherein the applying the energy source comprises stimulation with blue light at an intensity of at least 6.2 mW/cm².

70. The composition according to any one of paragraphs 59-69, wherein the DNA binding domain comprises (X₁₋₁₁-X₁₂X₁₃-X_{14-33 or 34 or 35})_{z},

wherein X₁₋₁₁ is a chain of 11 contiguous amino acids,

wherein X₁₂X₁₃ is a repeat variable diresidue (RVD),

wherein X₁₄₋₃₃ or 34 or 35 is a chain of 21, 22 or 23 contiguous amino acids,

wherein z is at least 5 to 40, and

wherein at least one RVD is selected from the group consisting of NI, HD, NG, NN, KN, RN, NH, NQ, SS, SN, NK, KH, RH, HH, HI, KI, RI, SI, KG, HG, RG, SD, ND, KD, RD, YG, HN, NV, NS, HA, S*, N*, KA, H*, RA, NA, and NC, wherein (*) means that the amino acid at X₁₃ is absent.

71. The composition according to paragraph 70, wherein z is at least 10 to 26.

72. The composition according to paragraph 70, wherein

at least one of X₁₋₁₁ is a sequence of 12 contiguous amino acids set forth as amino acids 1-11 in a sequence (X₁₋₁₁-X₁₄₋₃₄ or X₁₋₁₁-X₁₄₋₃₅) of FIG. 9 or

at least one of X₁₄₋₃₄ or X₁₄₋₃₅ is a sequence of 21 or 22 contiguous amino acids set forth as amino acids 12-32 or 12-33 in a sequence (X₁₁₁-X₁₄₋₃₄or X₁₋₁₁-X₁₄₋₃₅) of FIG. 9.

73. The composition according to paragraph 70, wherein the at least one RVD is selected from the group consisting of (a) HH, KH, NH, NK, NQ, RH, RN, SS for recognition of guanine (G); (b) SI for recognition of adenine (A); (c) HG, KG, RG for recognition of thymine (T); (d) RD, SD for recognition of cytosine (C); (e) NV for recognition of A or G; and (f) H*, HA, KA, N*, NA, NC, NS, RA, S* for recognition of A or T or G or C, wherein (*) means that the amino acid at X₁₃ is absent.

74. The composition according to paragraph 73, wherein
the RVD for the recognition of G is RN, NH, RH or KH; or
the RVD for the recognition of A is SI; or
the RVD for the recognition of T is KG or RG; and
the RVD for the recognition of C is SD or RD.

75. The composition according to paragraph 70, wherein at least one of the following is present
[LTLD] (SEQ ID NO: 1) or [LTLA] (SEQ ID NO: 2) or [LTQV] (SEQ ID NO: 3) at X₁₋₄, or
[EQHG] (SEQ ID NO: 4) or [RDHG] (SEQ ID NO: 5) at positions X₃₀₋₃₃ or X₃₁₋₃₄ or X₃₂₋₃₅.

76. The composition according to any one of paragraphs 59-75, wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

77. The composition according to any one of paragraphs 59-76, wherein the genomic locus of interest is associated with a gene that encodes for a differentiation factor, a transcription factor, a neurotransmitter transporter, a neurotransmitter synthase, a synaptic protein, a plasticity protein, a presynaptic active zone protein, a post synaptic density protein, a neurotransmitter receptor, an epigenetic modifier, a neural fate specification factor, an axon guidance molecule, an ion channel, a CpG binding protein, a ubiquitination protein, a hormone, a homeobox protein, a growth factor, an oncogenes or a proto-oncogene.

78. A non-naturally occurring or engineered composition for inducibly repressing expression of a genomic locus in a cell wherein the composition comprises a DNA binding polypeptide comprising:
(i) a DNA binding domain comprising at least one or more TALE monomers or half-monomers or
at least one or more repressor domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers or
at least one or more repressor domains
linked to the interacting partner, wherein the energy sensitive protein or fragment thereof binds to the interacting partner upon induction by the energy source;

wherein the polypeptide is encoded by and expressed from a codon optimized nucleic acid molecule so that the polypeptide preferentially binds to DNA of the genomic locus, and

wherein the polypeptide represses the expression of the genomic locus upon application of the energy source.

79. The composition according to paragraph 78, wherein the polypeptide includes at least one SID repressor domain.

80. The composition according to paragraph 79, wherein the polypeptide includes at least four SID repressor domains.

81. The composition according to paragraph 78, wherein the polypeptide includes a SID4X repressor domain.

82. The composition according to paragraph 78, wherein the polypeptide includes a KRAB repressor domain.

83. The composition according to any one of paragraphs 78-82, wherein the energy sensitive protein is Cryptochrome-2 (CRY2).

84. The composition according to any one of paragraphs 78-83, wherein the interacting partner is Cryptochrome-interacting basic helix-loop-helix (CIB1).

85. The composition according to any one of paragraphs 78-84, wherein the energy source is selected from the group consisting of: electromagnetic radiation, sound energy or thermal energy.

86. The composition according to paragraph 78, wherein the electromagnetic radiation is a component of visible light.

87. The composition according to paragraph 86, wherein the component of visible light has a wavelength in the range of 450 nm-500 nm.

88. The composition according to paragraph 86, wherein the component of visible light is blue light.

89. The composition according to paragraph 78, wherein the applying the energy source comprises stimulation with blue light at an intensity of at least 6.2 mW/cm².

90. The composition according to any one of paragraphs 78-89, wherein the DNA binding domain comprises (X₁₋₁₁-X₁₂X₁₃-X_{14-33 or 34 or 35})_{z},

wherein X₁₋₁₁ is a chain of 11 contiguous amino acids,

wherein X₁₂X₁₃ is a repeat variable diresidue (RVD),

wherein X_{14-33 or 34 or 35} is a chain of 21, 22 or 23 contiguous amino acids,

wherein z is at least 5 to 40, and

wherein at least one RVD is selected from the group consisting of NI, HD, NG, NN, KN, RN, NH, NQ, SS, SN, NK, KH, RH, HH, HI, KI, RI, SI, KG, HG, RG, SD, ND, KD, RD, YG, HN, NV, NS, HA, S*, N*, KA, H*, RA, NA, and NC, wherein (*) means that the amino acid at X₁₃ is absent.

91. The composition according to paragraph 90, wherein z is at least 10 to 26.

92. The composition according to paragraph 90, wherein

at least one of X₁₋₁₁ is a sequence of 11 contiguous amino acids set forth as amino acids 1-11 in a sequence (X₁₋₁₁-X₁₄₋₃₄ or X₁₋₁₁-X₁₄₋₃₅) of FIG. 9 or at least one of X₁₄₋₃₄ or X₁₄₋₃₅ is a sequence of 21 or 22 contiguous amino acids set forth as amino acids 12-32 or 12-33 in a sequence (X₁₋₁₁-X₁₄₋₃₄ or X₁₋₁₁-X₁₄₋₃₅) of FIG. 9.

93. The composition according to any one of paragraphs 78-92, wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

94. The composition according to any of paragraphs 78-93, wherein the genomic locus of interest is the genomic locus associated with a gene that encodes for a differentiation factor or a component of an ion channel.

95. The composition according to paragraph 94, wherein the differentiation factor is SRY-box-2 (SOX2) and is encoded by the gene SOX2.

96. The composition according to paragraph 94, wherein the differentiation factor is p11 and is encoded by the gene p11.

97. The composition according to paragraph 95, wherein the component of the ion channel is CACNA1C and is encoded by the gene CACNA1C.

98. A non-naturally occurring or engineered composition for inducibly activating expression of a genomic locus of interest in a cell wherein the composition comprises a DNA binding polypeptide comprising:
(i) a DNA binding domain comprising at least five or more Transcription activator-like effector (TALE) monomers and at least one or more half-monomers specifically ordered to target the genomic locus of interest or
at least one or more effector domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers specifically ordered to target the genomic locus of interest or
at least one or more effector domains
linked to the interacting partner, wherein the energy sensitive protein or fragment thereof binds to the interacting partner upon induction by the energy source;

wherein the polypeptide is encoded by and expressed from a codon optimized nucleic acid molecule so that the polypeptide preferentially binds to DNA of the genomic locus, and

wherein the polypeptide activates the expression of the genomic locus upon application of the energy source.

99. The composition according to paragraph 98, wherein the polypeptide includes at least one VP16 or VP64 activator domain.

100. The composition according to paragraph 98, wherein the polypeptide includes at least one p65 activator domain.

101. The composition according to any one of paragraphs 98-100, wherein the energy sensitive protein is CRY2.

102. The composition according to any one of paragraphs 98-101, wherein the interacting partner is CIB1.

103. The composition according to paragraph 98, wherein the energy source is selected from the group consisting of: electromagnetic radiation, sound energy or thermal energy.

104. The composition according to paragraph 103, wherein the electromagnetic radiation is a component of visible light.

105. The method according to paragraph 104, wherein the component of visible light has a wavelength in the range of 450 nm-500 nm.

106. The method according to paragraph 104, wherein the component of visible light is blue light.

107. The method according to paragraph 98, wherein the applying the energy source comprises stimulation with blue light at an intensity of at least 6.2 mW/cm².

108. The composition according to any one of paragraphs 98-107, wherein the DNA binding domain comprises (X₁₋₄₁-X₁₂X₁₃-X₁₄₋₃₃ or 34 or 35)_{z},

wherein X₁₋₁₁ is a chain of 11 contiguous amino acids,

wherein X₁₂X₁₃ is a repeat variable diresidue (RVD),

wherein X₁₄₋₃₃ or 34 or 35 is a chain of 21, 22 or 23 contiguous amino acids,

wherein z is at least 5 to 40, and

wherein at least one RVD is selected from the group consisting of NI, HD, NG, NN, KN, RN, NH, NQ, SS, SN, NK, KH, RH, HH, HI, KI, RI, SI, KG, HG, RG, SD, ND, KD, RD, YG, HN, NV, NS, HA, S*, N*, KA, H*, RA, NA, and NC, wherein (*) means that the amino acid at X₁₃ is absent.

109. The composition according to paragraph 108, wherein z is at least 10 to 26.

110. The composition according to paragraph 108, wherein

at least one of X₁₋₁₁ is a sequence of 11 contiguous amino acids set forth as amino acids 1-11 in a sequence (X₁₋₁₁-X₁₄₋₃₄ or X₁₋₁₁-X₁₄₋₃₅) of FIG. 9 or at least one of X₁₄₋₃₄ or X₁₄₋₃₅ is a sequence of 21 or 22 contiguous amino acids set forth as amino acids 12-32 or 12-33 in a sequence (X₁₋₁₁-X₁₄₋₃₄ or X₁₋₁₁-X₁₄₋₃₅) of FIG. 9.

111. The composition according to any one of paragraphs 98-110, wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

112. The composition according to any one of paragraphs 98-111, wherein the genomic locus of interest is the genomic locus associated with a gene that encodes for a differentiation factor, an epigenetic modulator, a component of an ion channel or a receptor.

113. The composition according to paragraph 112, wherein the differentiation factor is Neurogenin-2 and is encoded by the gene NEUROG2.

114. The composition according to paragraph 112, wherein the differentiation factor is Kreuppel-like factor 4 and is encoded by the gene KLF-4.

115. The composition according to paragraph 112, wherein the epigenetic modulator is Tet methylcytosine dioxygenase 1 and is encoded by the gene tet-1.

116. The composition according to paragraph 112, wherein the component of the ion channel is CACNA1C and is encoded by the gene CACNA1C.

117. The composition according to paragraph 112, wherein the receptor is metabotropic glutamate receptor and is encoded by the gene mGlur2.

118. The composition according to any one of paragraphs 98-117, wherein the expression is chemically inducible.

119. The composition according to paragraph 118, wherein the chemically inducible expression system is an estrogen based (ER) system inducible by 4-hydroxytamoxifen (4OHT).

120. The composition according to paragraph 119 wherein the composition further comprises a nuclear exporting signal (NES).

121. The composition of paragraph 120, wherein the NES has the sequence of LDLASLIL (SEQ ID NO: 6).

122. A nucleic acid encoding the composition according to any one of paragraphs 98-121.

123. The nucleic acid of paragraph 122 wherein the nucleic acid comprises a promoter.

124. The nucleic acid according to paragraph 123, wherein the promoter is a human Synapsin I promoter (hSyn).

125. The nucleic acid according to any one of paragraphs 122-124, wherein the nucleic acid is packaged into an adeno associated viral vector (AAV).

126. An inducible method of altering expression of a genomic locus of interest comprising:

(a) contacting the genomic locus with a non-naturally occurring or engineered composition comprising a DNA binding polypeptide comprising:
(i) a DNA binding domain comprising at least five or more Transcription activator-like effector (TALE) monomers and at least one or more half-monomers specifically ordered to target the genomic locus of interest or
at least one or more effector domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers specifically ordered to target the genomic locus of interest or
at least one or more effector domains
linked to the interacting partner, wherein the energy sensitive protein or fragment thereof binds to the interacting partner upon induction by the energy source;

(b) applying the energy source; and

(c) determining that the expression of the genomic locus is altered.

127. A non-naturally occurring or engineered composition for inducibly altering expression of a genomic locus wherein the composition comprises a DNA binding polypeptide comprising:
(i) a DNA binding domain comprising at least five or more Transcription activator-like effector (TALE) monomers and at least one or more half-monomers specifically ordered to target the genomic locus of interest or
at least one or more effector domains
linked to an energy sensitive protein or fragment thereof, wherein the energy sensitive protein or fragment thereof undergoes a conformational change upon induction by an energy source allowing it to bind an interacting partner, and/or
(ii) a DNA binding domain comprising at least one or more TALE monomers or half-monomers specifically ordered to target the genomic locus of interest or
at least one or more effector domains
linked to the interacting partner, wherein the energy sensitive protein or fragment thereof binds to the interacting partner upon induction by the energy source;

wherein the polypeptide preferentially binds to DNA of the genomic locus, and

wherein the polypeptide alters the expression of the genomic locus upon application of the energy source.

128. An inducible method for perturbing expression of a genomic locus of interest in a cell comprising:

(a) contacting the genomic locus with a non-naturally occurring or engineered composition comprising a deoxyribonucleic acid (DNA) binding polypeptide;

(b) applying an inducer source; and

(c) determining that perturbing expression of the genomic locus has occurred.

129. The method of paragraph 128, wherein perturbing expression is altering expression (up or down), altering the expression result (such as with nuclease) or eliminating expression shifting, for example, altering expression to dependent option.

130. The method of paragraph 128 or 129, wherein the inducer source is an energy source (such as wave or heat) or a small molecule.

131. The method of any one of paragraphs 126-130, wherein the DNA binding polypeptide comprises:

(i) a DNA binding domain comprising at least five or more Transcription activator-like effector (TALE) monomers and at least one or more half-monomers specifically ordered to target the genomic locus of interest or at least one or more effector domains linked to an energy sensitive protein or fragment thereof, wherein the energy sensitive protein or fragment thereof undergoes a conformational change upon induction by an energy source allowing it to bind an interacting partner, and/or

(ii) a DNA binding domain comprising at least one or more TALE monomers or half-monomers specifically ordered to target the genomic locus of interest or at least one or more effector domains linked to the interacting partner, wherein the energy sensitive protein or fragment thereof binds to the interacting partner upon induction by the energy source.

132. An inducible method for perturbing expression of a genomic locus of interest in a cell comprising:

(a) contacting the genomic locus with a vector system comprising one or more vectors comprising

I. a first regulatory element operably linked to a CRISPR/Cas system chimeric RNA (chiRNA) polynucleotide sequence, wherein the polynucleotide sequence comprises

(a) a guide sequence capable of hybridizing to a target sequence in a eukaryotic cell,

(b) a tracr mate sequence, and

(c) a tracr sequence, and

II. a second regulatory inducible element operably linked to an enzyme-coding sequence encoding a CRISPR enzyme comprising at least one or more nuclear localization sequences,

wherein (a), (b) and (c) are arranged in a 5′ to 3′orientation,

wherein components I and II are located on the same or different vectors of the system,

wherein when transcribed, the tracr mate sequence hybridizes to the tracr sequence and the guide sequence directs sequence-specific binding of a CRISPR complex to the target sequence, and

wherein the CRISPR complex comprises the CRISPR enzyme complexed with (1) the guide sequence that is hybridized to the target sequence, and (2) the tracr mate sequence that is hybridized to the tracr sequence,

wherein the enzyme coding sequence encoding the CRISPR enzyme further encodes a heterologous functional domain;

(b) applying an inducer source; and

(c) determining that perturbing expression of the genomic locus has occurred.

133. The method of paragraph 132, wherein perturbing expression is altering expression (up or down), altering the expression result (such as with nuclease) or eliminating expression shifting, for example, altering expression to dependent option.

134. The method of paragraph 132 or 133, wherein the inducer source is a chemical.

135. The method of any one of paragraphs 132 to 134, wherein the vector is a lentivirus.

136. The method of any one of paragraphs 132 to 135, wherein the second regulatory inducible element comprises a tetracycline-dependent regulatory system.

137. The method of any one of paragraphs 132 to 135, wherein the second regulatory inducible element comprises a cumate gene switch system.

138. The composition, nucleic acid or method of any one of paragraphs 1-137, wherein the cell is an a prokaryotic cell or a eukaryotic cell.

139. The composition, nucleic acid or method of paragraph 138, wherein the eukaryotic cell is an animal cell.

140. The composition, nucleic acid or method of paragraph 139, wherein the animal cell is a mammalian cell.

201. A non-naturally occurring or engineered TALE or CRISPR-Cas system, comprising at least one switch wherein the activity of said TALE or CRISPR-Cas system is controlled by contact with at least one inducer energy source as to the switch.

202. The system according to paragraph 201 wherein the control as to the at least one switch or the activity of said TALE or CRISPR-Cas system is activated, enhanced, terminated or repressed.

203. The system according to any of the preceding paragraphs wherein contact with the at least one inducer energy source results in a first effect and a second effect.

204. The system according to paragraph 203 wherein the first effect is one or more of nuclear import, nuclear export, recruitment of a secondary component (such as an effector molecule), conformational change (of protein, DNA or RNA), cleavage, release of cargo (such as a caged molecule or a co-factor), association or dissociation.

205. The system according to paragraph 203 wherein the second effect is one or more of activation, enhancement, termination or repression of the control as to the at least one switch or the activity of said TALE or CRISPR-Cas system.

206. The system according to any of paragraphs 203-205 wherein the first effect and the second effect occur in a cascade.

207. The system according to any of the preceding paragraphs wherein said TALE or CRISPR-Cas system further comprises at least one nuclear localization signal (NLS), nuclear export signal (NES), functional domain, flexible linker, mutation, deletion, alteration or truncation.

208. The system according to paragraph 207 wherein one or more of the NLS, the NES or the functional domain is conditionally activated or inactivated.

209. The system according to paragraph 207 wherein the mutation is one or more of a mutation in a transcription factor homology region, a mutation in a DNA binding domain (such as mutating basic residues of a basic helix loop helix), a mutation in an endogenous NLS or a mutation in an endogenous NES.

210. The system according to any of the preceding paragraphs wherein the inducer energy source is heat, ultrasound, electromagnetic energy or chemical.

211. The system according to any of the preceding paragraphs wherein the inducer energy source is an antibiotic, a small molecule, a hormone, a hormone derivative, a steroid or a steroid derivative.

212. The system according to any of the preceding paragraphs wherein the inducer energy source is abscisic acid (ABA), doxycycline (DOX), cumate, rapamycin, 4-hydroxytamoxifen (4OHT), estrogen or ecdysone.

213. The system according to any one of the preceding paragraphs wherein the at least one switch is selected from the group consisting of antibiotic based inducible systems, electromagnetic energy based inducible systems, small molecule based inducible systems, nuclear receptor based inducible systems and hormone based inducible systems.

214. The system according to any one of the preceding paragraphs wherein the at least one switch is selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems.

215. The system according to paragraph 210 wherein the inducer energy source is electromagnetic energy.

216. The system according to paragraph 215 wherein the electromagnetic energy is a component of visible light.

217. The system according to paragraph 216 wherein the component of visible light has a wavelength in the range of 450 nm-700 nm.

218. The system according to paragraph 217 wherein the component of visible light has a wavelength in the range of 450 nm-500 nm.

219. The system according to paragraph 218 wherein the component of visible light is blue light.

220. The system according to paragraph 219 wherein the blue light has an intensity of at least 0.2 mW/cm².

221. The system according to paragraph 219 wherein the blue light has an intensity of at least 4 mW/cm².

222. The system according to paragraph 217 wherein the component of visible light has a wavelength in the range of 620-700 nm.

223. The system according to paragraph 222 wherein the component of visible light is red light.

224. The system according to paragraph 207 wherein the at least one functional domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease.

225. Use of the system in any of the preceding paragraphs for perturbing a genomic or epigenomic locus of interest.

226. Use of the system in any of paragraphs 201-224 for the preparation of a pharmaceutical compound.

227. A method of controlling a non-naturally occurring or engineered TALE or CRISPR-Cas system, comprising providing said TALE or CRISPR-Cas system comprising at least one switch wherein the activity of said TALE or CRISPR-Cas system is controlled by contact with at least one inducer energy source as to the switch.

228. The method according to paragraph 227 wherein the control as to the at least one switch or the activity of said TALE or CRISPR-Cas system is activated, enhanced, terminated or repressed.

229. The method according to paragraphs 227 or 228 wherein contact with the at least one inducer energy source results in a first effect and a second effect.

230. The method according to paragraph 229 wherein the first effect is one or more of nuclear import, nuclear export, recruitment of a secondary component (such as an effector molecule), conformational change (of protein, DNA or RNA), cleavage, release of cargo (such as a caged molecule or a co-factor), association or dissociation.

231. The method according to paragraph 229 wherein the second effect is one or more of activation, enhancement, termination or repression of the control as to the at least one switch or the activity of said TALE or CRISPR-Cas system.

232. The method according to any of paragraphs 229-231 wherein the first effect and the second effect occur in a cascade.

233. The method according to any of paragraphs 227-232 wherein said TALE or CRISPR-Cas system further comprises at least one nuclear localization signal (NLS), nuclear export signal (NES), functional domain, flexible linker, mutation, deletion, alteration or truncation.

234. The method according to paragraph 233 wherein one or more of the NLS, the NES or the functional domain is conditionally activated or inactivated.

235. The method according to paragraph 233 wherein the mutation is one or more of a mutation in a transcription factor homology region, a mutation is a DNA binding domain (such as mutating basic residues of a basic helix loop helix), a mutation in an endogenous NLS or a mutation in an endogenous NES.

236. The method according to any of paragraphs 227-235 wherein the inducer energy source is heat, ultrasound, electromagnetic energy or chemical.

237. The method according to any of paragraphs 227-236 wherein the inducer energy source is an antibiotic, a small molecule, a hormone, a hormone derivative, a steroid or a steroid derivative.

238. The method according to any of paragraphs 227-237 wherein the inducer energy source is abscisic acid (ABA), doxycycline (DOX), cumate, rapamycin, 4-hydroxytamoxifen (4OHT), estrogen or ecdysone.

239. The method according to any of paragraphs 227-238 wherein the at least one switch is selected from the group consisting of antibiotic based inducible systems, electromagnetic energy based inducible systems, small molecule based inducible systems, nuclear receptor based inducible systems and hormone based inducible systems.

240. The method according to any of paragraphs 227-239 wherein the at least one switch is selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems.

241. The method according to paragraph 236 wherein the inducer energy source is electromagnetic energy.

242. The method according to paragraph 241 wherein the electromagnetic energy is a component of visible light.

243. The method according to paragraph 242 wherein the component of visible light has a wavelength in the range of 450 nm-700 nm.

244. The method according to paragraph 243 wherein the component of visible light has a wavelength in the range of 450 nm-500 nm.

245. The method according to paragraph 244 wherein the component of visible light is blue light.

246. The method according to paragraph 245 wherein the blue light has an intensity of at least 0.2 mW/cm².

247. The method according to paragraph 245 wherein the blue light has an intensity of at least 4 mW/cm².

248. The method according to paragraph 243 wherein the component of visible light has a wavelength in the range of 620-700 nm.

249. The method according to paragraph 248 wherein the component of visible light is red light.

250. The method according to paragraph 233 wherein the at least one functional domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease.

251. The system or method according to any of the preceding paragraphs wherein the TALE system comprises a DNA binding polypeptide comprising:
(i) a DNA binding domain comprising at least five or more Transcription activator-like effector (TALE) monomers and at least one or more half-monomers specifically ordered to target a locus of interest or
   at least one or more effector domains
   linked to an energy sensitive protein or fragment thereof, wherein the energy sensitive protein or fragment thereof undergoes a conformational change upon induction by an inducer energy source allowing it to bind an interacting partner, and/or
(ii) a DNA binding domain comprising at least one or more TALE monomers or half-monomers specifically ordered to target the locus of interest or
   at least one or more effector domains
   linked to the interacting partner, wherein the energy sensitive protein or fragment thereof binds to the interacting partner upon induction by the inducer energy source.

252. The system or method of paragraph 251 wherein the DNA binding polypeptide comprises
(a) a N-terminal capping region
(b) a DNA binding domain comprising at least 5 to 40 Transcription activator-like effector (TALE) monomers and at least one or more half-monomers specifically ordered to target the locus of interest, and
(c) a C-terminal capping region

wherein (a), (b) and (c) are arranged in a predetermined N-terminus to C-terminus orientation,

wherein the genomic locus comprises a target DNA sequence 5′-T₀N₁N₂ . . . N_{z} N_{z+1}-3′, where T₀ and N=A, G, T or C,

wherein the target DNA sequence binds to the DNA binding domain, and the DNA binding domain comprises (X₁₋₁₁-X₁₂X₁₃-X_{14-33 or 34 or 35})_{z},

wherein X₁₋₁₁ is a chain of 11 contiguous amino acids,

wherein X₁₂X₁₃ is a repeat variable diresidue (RVD),

wherein X_{14-33 or 34 or 35} is a chain of 21, 22 or 23 contiguous amino acids,

wherein z is at least 5 to 40,

wherein the polypeptide is encoded by and translated from a codon optimized nucleic acid molecule so that the polypeptide preferentially binds to DNA of the locus of interest.

253. The system or method of paragraph 252 wherein
the N-terminal capping region or fragment thereof comprises 147 contiguous amino acids of a wild type N-terminal capping region, or
the C-terminal capping region or fragment thereof comprises 68 contiguous amino acids of a wild type C-terminal capping region, or
the N-terminal capping region or fragment thereof comprises 136 contiguous amino acids of a wild type N-terminal capping region and the C-terminal capping region or fragment thereof comprises 183 contiguous amino acids of a wild type C-terminal capping region.

254. The system or method of paragraph 252 wherein at least one RVD is selected from the group consisting of (a) HH, KH, NH, NK, NQ, RH, RN, SS, NN, SN, KN for recognition of guanine (G); (b) NI, KI, RI, HI, SI for recognition of adenine (A); (c) NG, HG, KG, RG for recognition of thymine (T); (d) RD, SD, HD, ND, KD, YG for recognition of cytosine (C); (e) NV, HN for recognition of A or G; and (f) H*, HA, KA, N*, NA, NC, NS, RA, S*for recognition of A or T or G or C, wherein (*) means that the amino acid at X₁₃ is absent.

255. The system or method of paragraph 254 wherein at least one RVD is selected from the group consisting of (a) HH, KH, NH, NK, NQ, RH, RN, SS for recognition of guanine (G); (b) SI for recognition of adenine (A); (c) HG, KG, RG for recognition of thymine (T); (d) RD, SD for recognition of cytosine (C); (e) NV, HN for recognition of A or G and (f) H*, HA, KA, N*, NA, NC, NS, RA, S*for recognition of A or T or G or C, wherein (*) means that the amino acid at X₁₃ is absent.

256. The system or method of paragraph 255 wherein
the RVD for the recognition of G is RN, NH, RH or KH; or
the RVD for the recognition of A is SI; or
the RVD for the recognition of T is KG or RG; and
the RVD for the recognition of C is SD or RD.

257. The system or method of paragraph 252 wherein at least one of the following is present

[LTLD] (SEQ ID NO: 1) or [LTLA] (SEQ ID NO: 2) or [LTQV] (SEQ ID NO: 3) at X₁₋₄, or
[EQHG] (SEQ ID NO: 4) or [RDHG] (SEQ ID NO: 5) at positions X₃₀₋₃₃ or X₃₁₋₃₄ or X₃₂₋₃₅.

258. The system or method according to any of paragraphs 251-257 wherein the TALE system is packaged into a AAV or a lentivirus vector.

259. The system or method according to any of paragraphs 201-250 wherein the CRISPR system comprises a vector system comprising:

a) a first regulatory element operably linked to a CRISPR-Cas system guide RNA that targets a locus of interest,

b) a second regulatory inducible element operably linked to a Cas protein,

wherein components (a) and (b) are located on same or different vectors of the system,

wherein the guide RNA targets DNA of the locus of interest, wherein the Cas protein and the guide RNA do not naturally occur together.

260. The system or method according to paragraph 259 wherein the Cas protein is a Cas9 enzyme.

261. The system or method according to paragraphs 259 or 260 wherein the vector is AAV or lentivirus.

301. A vector system comprising one or more vectors, wherein the system comprises

a. a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and

b. a second regulatory element operably linked to an enzyme-coding sequence encoding said CRISPR enzyme comprising a nuclear localization sequence;

wherein components (a) and (b) are located on the same or different vectors of the system.

302. The vector system of paragraph 301, wherein component (a) further comprises the tracr sequence downstream of the tracr mate sequence under the control of the first regulatory element.

303. The vector system of paragraph 301, wherein component (a) further comprises two or more guide sequences operably linked to the first regulatory element, wherein when expressed, each of the two or more guide sequences direct sequence specific binding of a CRISPR complex to a different target sequence in a eukaryotic cell.

304. The vector system of paragraph 301, wherein the system comprises the tracr sequence under the control of a third regulatory element.

305. The vector system of paragraph 301, wherein the tracr sequence exhibits at least 50% of sequence complementarity along the length of the tracr mate sequence when optimally aligned.

306. The vector system of paragraph 301, wherein the CRISPR enzyme comprises one or more nuclear localization sequences of sufficient strength to drive accumulation of said CRISPR enzyme in a detectable amount in the nucleus of a eukaryotic cell.

307. The vector system of paragraph 301, wherein the CRISPR enzyme is a type II CRISPR system enzyme.

308. The vector system of paragraph 301, wherein the CRISPR enzyme is a Cas9 enzyme.

309. The vector system of paragraph 301, wherein the CRISPR enzyme is codon-optimized for expression in a eukaryotic cell.

310. The vector system of paragraph 301, wherein the CRISPR enzyme directs cleavage of one or two strands at the location of the target sequence.

311. The vector system of paragraph 301, wherein the CRISPR enzyme lacks DNA strand cleavage activity.

312. The vector system of paragraph 301, wherein the first regulatory element is a polymerase III promoter.

313. The vector system of paragraph 301, wherein the second regulatory element is a polymerase II promoter.

314. The vector system of paragraph 304, wherein the third regulatory element is a polymerase III promoter.

315. The vector system of paragraph 301, wherein the guide sequence is at least 15 nucleotides in length.

316. The vector system of paragraph 301, wherein fewer than 50% of the nucleotides of the guide sequence participate in self-complementary base-pairing when optimally folded.

317. A vector comprising a regulatory element operably linked to an enzyme-coding sequence encoding a CRISPR enzyme comprising one or more nuclear localization sequences, wherein said regulatory element drives transcription of the CRISPR enzyme in a eukaryotic cell such that said CRISPR enzyme accumulates in a detectable amount in the nucleus of the eukaryotic cell.

318. The vector of paragraph 317, wherein said regulatory element is a polymerase II promoter.

319. The vector of paragraph 317, wherein said CRISPR enzyme is a type II CRISPR system enzyme.

320. The vector of paragraph 317, wherein said CRISPR enzyme is a Cas9 enzyme.

321. The vector of paragraph 317, wherein said CRISPR enzyme lacks the ability to cleave one or more strands of a target sequence to which it binds.

322. A CRISPR enzyme comprising one or more nuclear localization sequences of sufficient strength to drive accumulation of said CRISPR enzyme in a detectable amount in the nucleus of a eukaryotic cell.

323. The CRISPR enzyme of paragraph 322, wherein said CRISPR enzyme is a type II CRISPR system enzyme.

324. The CRISPR enzyme of paragraph 322, wherein said CRISPR enzyme is a Cas9 enzyme.

325. The CRISPR enzyme of paragraph 322, wherein said CRISPR enzyme lacks the ability to cleave one or more strands of a target sequence to which it binds.

326. A eukaryotic host cell comprising:

a. a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or

b. a second regulatory element operably linked to an enzyme-coding sequence encoding said CRISPR enzyme comprising a nuclear localization sequence.

327. The eukaryotic host cell of paragraph 326, wherein said host cell comprises components (a) and (b).

328. The eukaryotic host cell of paragraph 326, wherein component (a), component (b), or components (a) and (b) are stably integrated into a genome of the host eukaryotic cell.

329. The eukaryotic host cell of paragraph 326, wherein component (a) further comprises the tracr sequence downstream of the tracr mate sequence under the control of the first regulatory element.

330. The eukaryotic host cell of paragraph 326, wherein component (a) further comprises two or more guide sequences operably linked to the first regulatory element, wherein when expressed, each of the two or more guide sequences direct sequence specific binding of a CRISPR complex to a different target sequence in a eukaryotic cell.

331. The eukaryotic host cell of paragraph 326, further comprising a third regulatory element operably linked to said tracr sequence.

332. The eukaryotic host cell of paragraph 326, wherein the tracr sequence exhibits at least 50% of sequence complementarity along the length of the tracr mate sequence when optimally aligned.

333. The eukaryotic host cell of paragraph 326, wherein the CRISPR enzyme comprises one or more nuclear localization sequences of sufficient strength to drive accumulation of said CRISPR enzyme in a detectable mount in the nucleus of a eukaryotic cell.

334. The eukaryotic host cell of paragraph 326, wherein the CRISPR enzyme is a type II CRISPR system enzyme.

335. The eukaryotic host cell of paragraph 326, wherein the CRISPR enzyme is a Cas9 enzyme.

336. The eukaryotic host cell of paragraph 326, wherein the CRISPR enzyme is codon-optimized for expression in a eukaryotic cell.

337. The eukaryotic host cell of paragraph 326, wherein the CRISPR enzyme directs cleavage of one or two strands at the location of the target sequence.

338. The eukaryotic host cell of paragraph 326, wherein the CRISPR enzyme lacks DNA strand cleavage activity.

339. The eukaryotic host cell of paragraph 326, wherein the first regulatory element is a polymerase III promoter.

340. The eukaryotic host cell of paragraph 326, wherein the second regulatory element is a polymerase II promoter.

341. The eukaryotic host cell of paragraph 331, wherein the third regulatory element is a polymerase III promoter.

342. The eukaryotic host cell of paragraph 326, wherein the guide sequence is at least 15 nucleotides in length.

343. The eukaryotic host cell of paragraph 326, wherein fewer than 50% of the nucleotides of the guide sequence participate in self-complementary base-pairing when optimally folded.

344. A non-human animal comprising a eukaryotic host cell of any one of paragraphs 326-343.

345. A kit comprising a vector system and instructions for using said kit, the vector system comprising:

a. a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or

b. a second regulatory element operably linked to an enzyme-coding sequence encoding said CRISPR enzyme comprising a nuclear localization sequence.

346. The kit of paragraph 345, wherein said kit comprises components (a) and (b) located on the same or different vectors of the system.

347. The kit of paragraph 345, wherein component (a) further comprises the tracr sequence downstream of the tracr mate sequence under the control of the first regulatory element.

348. The kit of paragraph 345, wherein component (a) further comprises two or more guide sequences operably linked to the first regulatory element, wherein when expressed, each of the two or more guide sequences direct sequence specific binding of a CRISPR complex to a different target sequence in a eukaryotic cell.

349. The kit of paragraph 345, wherein the system comprises the tracr sequence under the control of a third regulatory element.

350. The kit of paragraph 345, wherein the tracr sequence exhibits at least 50% of sequence complementarity along the length of the tracr mate sequence when optimally aligned.

351. The kit of paragraph 345, wherein the CRISPR enzyme comprises one or more nuclear localization sequences of sufficient strength to drive accumulation of said CRISPR enzyme in a detectable mount in the nucleus of a eukaryotic cell.

352. The kit of paragraph 345, wherein the CRISPR enzyme is a type II CRISPR system enzyme.

353. The kit of paragraph 345, wherein the CRISPR enzyme is a Cas9 enzyme.

354. The kit of paragraph 345, wherein the CRISPR enzyme is codon-optimized for expression in a eukaryotic cell.

355. The kit of paragraph 345, wherein the CRISPR enzyme directs cleavage of one or two strands at the location of the target sequence.

356. The kit of paragraph 345, wherein the CRISPR enzyme lacks DNA strand cleavage activity.

357. The kit of paragraph 345, wherein the first regulatory element is a polymerase III promoter.

358. The kit of paragraph 345, wherein the second regulatory element is a polymerase II promoter.

359. The kit of paragraph 349, wherein the third regulatory element is a polymerase III promoter.

360. The kit of paragraph 345, wherein the guide sequence is at least 15 nucleotides in length.

361. The kit of paragraph 345, wherein fewer than 50% of the nucleotides of the guide sequence participate in self-complementary base-pairing when optimally folded.

362. A computer system for selecting a candidate target sequence within a nucleic acid sequence in a eukaryotic cell for targeting by a CRISPR complex, the system comprising:

a. a memory unit configured to receive and/or store said nucleic acid sequence; and

b. one or more processors alone or in combination programmed to (i) locate a CRISPR motif sequence within said nucleic acid sequence, and (ii) select a sequence adjacent to said located CRISPR motif sequence as the candidate target sequence to which the CRISPR complex binds.

363. The computer system of paragraph 362, wherein said locating step comprises identifying a CRISPR motif sequence located less than about 500 nucleotides away from said target sequence.

364. The computer system of paragraph 362, wherein said candidate target sequence is at least 10 nucleotides in length.

365. The computer system of paragraph 362, wherein the nucleotide at the 3′ end of the candidate target sequence is located no more than about 10 nucleotides upstream of the CRISPR motif sequence.

366. The computer system of paragraph 362, wherein the nucleic acid sequence in the eukaryotic cell is endogenous to the eukaryotic genome.

367. The computer system of clam 362, wherein the nucleic acid sequence in the eukaryotic cell is exogenous to the eukaryotic genome.

368. A computer-readable medium comprising codes that, upon execution by one or more processors, implements a method of selecting a candidate target sequence within a nucleic acid sequence in a eukaryotic cell for targeting by a CRISPR complex, said method comprising: (a) locating a CRISPR motif sequence within said nucleic acid sequence, and (b) selecting a sequence adjacent to said located CRISPR motif sequence as the candidate target sequence to which the CRISPR complex binds.

369. The computer-readable medium of paragraph 368, wherein said locating comprises locating a CRISPR motif sequence that is less than about 500 nucleotides away from said target sequence.

370. The computer-readable of paragraph 368, wherein said candidate target sequence is at least 10 nucleotides in length.

371. The computer-readable of paragraph 368, wherein the nucleotide at the 3′ end of the candidate target sequence is located no more than about 10 nucleotides upstream of the CRISPR motif sequence.

372. The computer-readable of paragraph 368, wherein the nucleic acid sequence in the eukaryotic cell is endogenous the eukaryotic genome.

373. The computer-readable of paragraph 368, wherein the nucleic acid sequence in the eukaryotic cell is exogenous to the eukaryotic genome.

374. A method of modifying a target polynucleotide in a eukaryotic cell, the method comprising allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

375. The method of paragraph 374, wherein said cleavage comprises cleaving one or two strands at the location of the target sequence by said CRISPR enzyme.

376. The method of paragraph 374, wherein said cleavage results in decreased transcription of a target gene.

377. The method of paragraph 374, further comprising repairing said cleaved target polynucleotide by homologous recombination with an exogenous template polynucleotide, wherein said repair results in a mutation comprising an insertion, deletion, or substitution of one or more nucleotides of said target polynucleotide.

378. The method of paragraph 377, wherein said mutation results in one or more amino acid changes in a protein expressed from a gene comprising the target sequence.

379. The method of paragraph 374, further comprising delivering one or more vectors to said eukaryotic cell, wherein the one or more vectors drive expression of one or more of: the CRISPR enzyme, the guide sequence linked to the tracr mate sequence, and the tracr sequence.

380. The method of paragraph 379, wherein said vectors are delivered to the eukaryotic cell in a subject.

381. The method of paragraph 374, wherein said modifying takes place in said eukaryotic cell in a cell culture.

382. The method of paragraph 374, further comprising isolating said eukaryotic cell from a subject prior to said modifying.

383. The method of paragraph 382, further comprising returning said eukaryotic cell and/or cells derived therefrom to said subject.

384. A method of modifying expression of a polynucleotide in a eukaryotic cell, the method comprising: allowing a CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence.

385. The method of paragraph 374, further comprising delivering one or more vectors to said eukaryotic cells, wherein the one or more vectors drive expression of one or more of: the CRISPR enzyme, the guide sequence linked to the tracr mate sequence, and the tracr sequence.

386. A method of generating a model eukaryotic cell comprising a mutated disease gene, the method comprising:

a. introducing one or more vectors into a eukaryotic cell, wherein the one or more vectors drive expression of one or more of: a CRISPR enzyme, a guide sequence linked to a tracr mate sequence, and a tracr sequence; and

b. allowing a CRISPR complex to bind to a target polynucleotide to effect cleavage of the target polynucleotide within said disease gene, wherein the CRISPR complex comprises the CRISPR enzyme complexed with (1) the guide sequence that is hybridized to the target sequence within the target polynucleotide, and (2) the tracr mate sequence that is hybridized to the tracr sequence, thereby generating a model eukaryotic cell comprising a mutated disease gene.

387. The method of paragraph 386, wherein said cleavage comprises cleaving one or two strands at the location of the target sequence by said CRISPR enzyme.

388. The method of paragraph 386, wherein said cleavage results in decreased transcription of a target gene.

389. The method of paragraph 386, further comprising repairing said cleaved target polynucleotide by homologous recombination with an exogenous template polynucleotide, wherein said repair results in a mutation comprising an insertion, deletion, or substitution of one or more nucleotides of said target polynucleotide.

390. The method of paragraph 389, wherein said mutation results in one or more amino acid changes in a protein expressed from a gene comprising the target sequence.

391. A method of developing a biologically active agent that modulates a cell signaling event associated with a disease gene, comprising:

a. contacting a test compound with a model cell of any one of paragraphs 386-390; and

b. detecting a change in a readout that is indicative of a reduction or an augmentation of a cell signaling event associated with said mutation in said disease gene, thereby developing said biologically active agent that modulates said cell signaling event associated with said disease gene.

392. A recombinant polynucleotide comprising a guide sequence upstream of a tracr mate sequence, wherein the guide sequence when expressed directs sequence-specific binding of a CRISPR complex to a corresponding target sequence present in a eukaryotic cell.

393. The recombinant polynucleotide of paragraph 389, wherein the target sequence is a viral sequence present in a eukaryotic cell.

394. The recombinant polynucleotide of paragraph 389, wherein the target sequence is a proto-oncogene or an oncogene.

401. An engineered, non-naturally occurring Clustered Regularly Interspersed Short Palindromic Repeats (CRISPR)-CRISPR associated (Cas) (CRISPR-Cas) vector system comprising one or more vectors comprising:

a) a first regulatory element operably linked to one or more nucleotide sequences encoding one or more CRISPR-Cas system polynucleotide sequences comprising a guide sequence, a tracr RNA, and a tracr mate sequence, wherein the guide sequence hybridizes with one or more target sequences in polynucleotide loci in a eukaryotic cell,

b) a second regulatory element operably linked to a nucleotide sequence encoding a Type II Cas9 protein,

wherein components (a) and (b) are located on same or different vectors of the system,

wherein the CRISPR-Cas system comprises at least one switch,

whereby the activity of the system to target the one or more polynucleotide loci is controlled.

402. The system of paragraph 401, wherein the CRISPR-Cas system comprises a trans-activating cr (tracr) sequence.

403. The system of paragraph 401, wherein the Cas9 protein is codon optimized for expression in the eukaryotic cell and/or the eukaryotic cell is a mammalian or human cell.

404. The system of paragraph 401, wherein the Cas9 protein comprises two or more mutations; or wherein the Cas9 protein comprises two or more mutations selected from the group consisting of D10A, E762A, H840A, N854A, N863A and D986A with reference to the position numbering of a Streptococcus pyogenes Cas9 protein

405. The system of paragraph 401, wherein the one or more vectors are viral vectors.

406. The system of paragraph 401, wherein the viral vectors are selected from the group consisting of retroviral, lentiviral, adenoviral, adeno-associated and herpes simplex viral vectors.

407. The system of paragraph 401, wherein the control as to the at least one switch or the activity of said system is activated, enhanced, terminated or repressed.

408. The system of paragraph 401, wherein the system further comprises comprises at least one nuclear localization signal (NLS), functional domain, flexible linker, mutation, deletion, alteration or truncation.

409. The system of paragraph 401, wherein the inducer energy source is heat, ultrasound, electromagnetic energy, or chemical, a small molecule, a hormone, abscisic acid (ABA), rapamycin, 4-hydroxytamoxifen (4OHT), estrogen or ecdysone.

410. The system of paragraph 401, wherein the at least one switch is an antibiotic based inducible system, electromagnetic energy based inducible system, small molecule based inducible system, nuclear receptor based inducible system, hormone based inducible system, tetracycline (Tet) inducible system, light inducible system, ABA inducible system, 4OHT/estrogen inducible system, ecdysone-based inducible system or a FKBP12/FRAP (FKBP12-rapamycin complex) inducible system.

411. The system according to paragraph 410 wherein the inducer energy source is electromagnetic energy.

412. The system according to paragraph 411 wherein the electromagnetic energy is a component of visible light.

413. The system according to paragraph 412 wherein the component of visible light is blue light.

414. The system according to paragraph 414 wherein the blue light has an intensity of at least 0.2 mW/cm².

415. The system according to paragraph 408 wherein the at least one functional domain is a transposase domain, integrase domain, recombinase domain, resolvase domain, invertase domain, protease domain, DNA methyltransferase domain, DNA demethylase domain, histone acetylase domain, histone deacetylases domain, nuclease domain, transcriptional repressor domain, transcriptional activator domain, nuclear-localization signal domains, or cellular signal domain.

416. A method of modulating activity of any one of the systems of paragraphs 401-415, comprising administering the inducer energy source to the system, wherein the activity of the system is controlled by contact with the inducer energy source.

417. An engineered, non-naturally occurring Transcription activator-like effector (TALE) system comprising a DNA binding polypeptide comprising:

a) a DNA binding domain comprising at least five or more Transcription activator-like effector (TALE) monomers and at least one or more half-monomers specifically ordered to target a locus of interest linked to an energy sensitive protein or fragment thereof, wherein the energy sensitive protein or fragment thereof undergoes a conformational change upon induction by an inducer energy source allowing it to bind an interacting partner, and/or

b) a DNA binding domain comprising at least one or more TALE monomers or half-monomers specifically ordered to target the locus of interest linked to the interacting partner, wherein the energy sensitive protein or fragment thereof binds to the interacting partner upon induction by the inducer energy source.

418. The system of paragraph 417, wherein the one or more vectors are viral vectors

419. The system of paragraph 417, wherein the viral vectors are selected from the group consisting of retroviral, lentiviral, adenoviral, adeno-associated and herpes simplex viral vectors.

420. The system of paragraph 417, wherein the control as to the at least one switch or the activity of said system is activated, enhanced, terminated or repressed.

421. The system of paragraph 417, wherein the system further comprises comprises at least one nuclear localization signal (NLS), functional domain, flexible linker, mutation, deletion, alteration or truncation.

422. The system of paragraph 417, wherein the inducer energy source is heat, ultrasound, electromagnetic energy, or chemical, a small molecule, a hormone, abscisic acid (ABA), rapamycin, 4-hydroxytamoxifen (4OHT), estrogen or ecdysone.

423. The system of paragraph 417, wherein the at least one switch is an antibiotic based inducible system, electromagnetic energy based inducible system, small molecule based inducible system, nuclear receptor based inducible system, hormone based inducible system, tetracycline (Tet) inducible system, light inducible system, ABA inducible system, 4OHT/estrogen inducible system, ecdysone-based inducible system or a FKBP12/FRAP (FKBP12-rapamycin complex) inducible system.

424. The system according to paragraph 423 wherein the inducer energy source is electromagnetic energy.

425. The system according to paragraph 424 wherein the electromagnetic energy is a component of visible light.

426. The system according to paragraph 425 wherein the component of visible light is blue light.

427. The system according to paragraph 426 wherein the blue light has an intensity of at least 0.2 mW/cm².

428. The system according to paragraph 421 wherein the at least one functional domain is selected from the group consisting of: transposase domain, integrase domain, recombinase domain, resolvase domain, invertase domain, protease domain, DNA methyltransferase domain, DNA demethylase domain, histone acetylase domain, histone deacetylases domain, nuclease domain, transcriptional repressor domain, transcriptional activator domain, nuclear-localization signal domains, or cellular signal domain.

429. A method of modulating activity of any one of the systems of paragraphs 417-428, comprising administering the inducer energy source to the system, wherein the activity of the system is controlled by contact with the inducer energy source.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A non-naturally occurring Clustered Regularly Interspersed Short Palindromic Repeats (CRISPR)-CRISPR associated (Cas) (CRISPR-Cas) system, wherein the CRISPR-Cas system comprises:
(a) a CRISPR-Cas system guide RNA that targets a locus of interest in a eukaryotic cell;
(b) a Cas9 protein is linked to a first member of a heterodimer pair or a functional fragment of a first member of a heterodimer pair; wherein the CRISPR-Cas system guide RNA complexes with the Cas9 protein to form a DNA binding polypeptide that specifically targets a locus of interest in a eukaryotic cell, and
(c) an effector domain, wherein the effector domain is linked to the second member of the heterodimer pair or a functional fragment of the second member of the heterodimer pair,
wherein dimerization of the heterodimer pair occurs upon exposure to an inducer energy source.

2. The system of claim 1, wherein the Cas9 protein is a Type II Cas9 protein.

3. The system of claim 1, wherein the sequence encoding a CRISPR-Cas system guide RNA that targets a locus of interest comprises:
(a) a guide sequence capable of hybridizing to a target sequence in a eukaryotic cell;
(b) a tracr mate sequence; and
(c) a tracr sequence.

4. The system of claim 1, wherein the sequence encoding a Cas9 protein further comprises at least one or more nuclear localization sequences.

5. The system of claim 1, wherein said TALE or CRISPR-Cas system further comprises at least one nuclear localization signal (NLS), nuclear export signal (NES), or a mutated version thereof.

6. The system of claim 5, wherein one or more of the NLS or the NES is conditionally activated or inactivated.

7. The system of claim 1, wherein the inducer energy source is electromagnetic energy.

8. The system of claim 7, wherein the electromagnetic energy is a component of visible light.

9. The system of claim 8, wherein the component of visible light has a wavelength in the range of 450 nm-700 nm.

10. The system of claim 9, wherein the component of visible light has a wavelength in the range of 450 nm-500 nm.

11. The system of claim 8, wherein the component of visible light is blue light.

12. The system of claim 8, wherein the visible light has an intensity of at least 0.2 mW/cm².

13. The system of claim 12, wherein the visible light has an intensity of at least 4 mW/cm².

14. The system of claim 8, wherein the component of visible light has a wavelength in the range of 620-700 nm.

15. The system of claim 14, wherein the component of visible light is red light.

16. The system of claim 1, wherein the effector domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease.

17. The system of claim 1, wherein the system is formulated into a pharmaceutical composition.

18. The system of claim 1, wherein the system is packaged into an AAV or a lentivirus vector.

19. A method of controlling a non-naturally occurring CRISPR-Cas system, comprising:
(i) providing a CRISPR-Cas system, wherein the CRISPR-Cas system comprises a vector system comprising:
(a) a CRISPR-Cas system guide RNA that targets a locus of interest in a eukaryotic cell;
(b) a Cas9 protein is linked to a first member of a heterodimer pair or a functional fragment of a first member of a heterodimer pair; wherein the CRISPR-Cas system guide RNA complexes with the Cas9 protein to form a DNA binding polypeptide that specifically targets a locus of interest in a eukaryotic cell, and
(c) an effector domain, wherein the effector domain is linked to the second member of the heterodimer pair or a functional fragment of the second member of the heterodimer pair,
wherein dimerization of the heterodimer pair occurs upon exposure to an inducer energy source; and
(ii) introducing or removing the inducer energy source to control the system.
